(19) United States
(12) Patent Application Publication
(10) Pub. No.: US 2025/0042919 A1
(43) Pub. Date: Feb. 6, 2025

(54) TETRACYCLIC OXAZEPINE COMPOUNDS AND USES THEREOF

(71) Applicant: Genentech, Inc., South San Francisco, CA (US)

(72) Inventors: Lewis J. Gazzard, Belmont, CA (US); Samantha Alyson Green, Burlingame, CA (US); Matthew Leo Landry, San Mateo, CA (US); Sushant Malhotra, Burlingame, CA (US); Michael Siu, Burlingame, CA (US); Steven Do, San Jose, CA (US); Yun-Xing Cheng, Beijing (CN); Limin Cheng, Beijing (CN); Jianfeng Xin, Beijing (CN); Mingtao He, Beijing (CN)

(73) Assignee: Genentech, Inc., South San Francisco, CA (US)

(21) Appl. No.: 18/882,588

(22) Filed: Sep. 11, 2024

Related U.S. Application Data
(63) Continuation of application No. 17/666,350, filed on Feb. 7, 2022, now abandoned.

(30) Foreign Application Priority Data
Feb. 9, 2021 (WO) PCT/CN2021/076369
Jan. 27, 2022 (WO) PCT/CN2022/074435

(51) Int. Cl. C07D 498/22; A61K 31/553; A61K 45/06; A61P 35/00; C07D 519/00

(52) U.S. Cl. CPC C07D 498/22; A61K 31/553; A61K 45/06; A61P 35/00; C07D 519/00

(57) ABSTRACT

Provided herein are tetracyclic oxazepinyl compounds useful in the treatment on cancers.

### CROSS-REFERENCE TO RELATED APPLICATIONS

This application claims priority to International Patent Application No. PCT/CN2022/074435, filed 27 Jan. 2022, and to International Patent Application No. PCT/CN2021/076369, filed 9 Feb. 2021, each of which is incorporated herein by reference in its entirety and for all purposes.

### FIELD OF INVENTION

Provided herein are tetracyclic compounds useful in the treatment of cancers comprising a KRas mutation, compositions of such compounds, and methods of treating cancers comprising a KRas mutation.

### BACKGROUND

Ras is a small GTP-binding protein that functions as a nucleotide-dependent switch for central growth signaling pathways. In response to extracellular signals, Ras is converted from a GDP-bound (Ras^{GDP}) to a GTP-bound (Ras^{GTP}) state, as catalyzed by guanine nucleotide exchange factors (GEFs), notably the SOS1 protein. Active Ras^{GTP} mediates its diverse growth-stimulating functions through its direct interactions with effectors including Raf, PI3K, and RaI guanine nucleotide dissociation stimulator. The intrinsic GTPase activity of Ras then hydrolyzes GTP to GDP to terminate Ras signaling. The Ras GTPase activity can be further accelerated by its interactions with GTPase-activating proteins (GAPs), including the neurofibromin 1 tumor suppressor.

Mutant Ras has a reduced GTPase activity, which prolongs its activated state, thereby promoting Ras-dependent signaling and cancer cell survival or growth. Mutation in Ras that affects its ability to interact with GAP or to convert GTP back to GDP will result in a prolonged activation of the protein and consequently a prolonged signal to the cell telling it to continue to grow and divide. Because these signals result in cell growth and division, overactive RAS signaling may ultimately lead to cancer. Mutations in any one of the three main isoforms of RAS (HRas, NRas, or KRas) genes are common events in human tumorigenesis. Among the three Ras isoforms (K, N, and H), KRas is most frequently mutated.

The most common KRas mutations are found at residue G12 and G13 in the P-loop and at residue Q61. G12D is a frequent mutation of KRas gene (glycine-12 to aspartate). Mutations of Ras in cancer are associated with poor prognosis. Inactivation of oncogenic Ras in mice results in tumor shrinkage. Thus, Ras is widely considered an oncology target of exceptional importance.

Accordingly, there is a pressing need for therapies for G12D mutant KRas mediated cancers.

### SUMMARY

Provided herein are solutions to the problems above and other problems in the art.

In a first aspect provided herein is a compound of formula (I) or a stereoisomer, atropisomer, tautomer, or pharmaceutically acceptable salt thereof as described herein.

In another aspect provided herein is a compound of formula (Ia), (Ib), (Ic), (Id), (Ie), (Ig), or (Ih), or a stereoisomer, atropisomer, tautomer, or pharmaceutically acceptable salt thereof as described herein.

In another aspect provided herein is a compound of formula (IIa), (IIb), (IIc), (IId), (IIe), (IIg), or (IIh), or a stereoisomer, atropisomer, tautomer, or pharmaceutically acceptable salt thereof as described herein.

In another aspect provided herein is a compound of formula (IIIa), (IIIb), (IIIc), (IIId), (IIIe), (IIIg), or (IIIh), or a stereoisomer, atropisomer, tautomer, or pharmaceutically acceptable salt thereof as described herein.

In another aspect provided herein is a compound or pharmaceutically acceptable salt thereof as set forth in Table 1.

In another aspect provided herein is a compound or pharmaceutically acceptable salt thereof as set forth in Table 2.

In another aspect provided herein is a pharmaceutical composition comprising a compound, stereoisomer, atropisomer, tautomer, or pharmaceutically acceptable salt thereof as described herein.

In another aspect provided herein is a method of treating a cancer comprising a KRas mutation, the method comprising administering to a patient having such cancer, a compound, stereoisomer, atropisomer, tautomer, or pharmaceutically acceptable salt thereof as described herein.

In another aspect provided herein is a method for regulating activity of a KRas mutant protein, the method comprising reacting the mutant protein with a compound, or stereoisomer, atropisomer, tautomer, or pharmaceutically acceptable salt thereof as described herein.

In another aspect provided herein is a method for inhibiting proliferation of a cell population, the method comprising contacting the cell population with a compound, or stereoisomer, atropisomer, tautomer, or pharmaceutically acceptable salt thereof as described herein.

In another aspect provided herein is a method for inhibiting tumor metastasis comprising administering to an individual in need thereof a therapeutically effective amount of the compound or stereoisomer, atropisomer, tautomer, or pharmaceutically acceptable salt thereof as described herein or a pharmaceutical composition as described herein to a subject in need thereof.

In another aspect provided herein is method for preparing a labeled KRas G12D mutant protein, the method comprising reacting a KRas G12D mutant protein with a labeled compound or stereoisomer, atropisomer, tautomer, or pharmaceutically acceptable salt thereof, as described here to result in the labeled KRas G12D mutant protein.

In another aspect provided herein is a process for synthesizing a compound or stereoisomer, atropisomer, tautomer, or pharmaceutically acceptable salt thereof as set forth herein.

### Definitions

Disclosed herein are tetracyclic oxazepine compounds as described herein or a stereoisomer, atropisomer, tautomer, or pharmaceutically acceptable salt thereof and pharmaceutical compositions thereof that, in certain embodiments, are inhibitors or modulators of mutant KRas. In certain instances, such compounds and compositions are inhibitors or modulators of mutant G12D KRas as provided herein. The compounds and compositions described herein are useful in treating diseases and disorders mediated by mutant KRas.

While the disclosure herein provides enumerated embodiments, it is understood that they are not intended to limit the compounds and methods described herein to those embodiments. On the contrary, the disclosure is intended to cover all alternatives, modifications, and equivalents that can be included within the scope of the present disclosure as defined by the claims.

Unless otherwise defined, all technical and scientific terms used herein have the same meaning as commonly understood by one of ordinary skill in the art. All publications, patent applications, patents, and other references mentioned herein are incorporated by reference in their entirety. The nomenclature used in this Application is based on IUPAC systematic nomenclature, unless indicated otherwise.

The following definitions are provided to facilitate understanding of certain terms used frequently herein and are not meant to limit the scope of the present disclosure. All references referred to herein are incorporated by reference in their entirety.

The terms “halogen” and “halo” are used interchangeably and refer to F, Cl, Br or I. Additionally, terms such as “haloalkyl,” are meant to include monohaloalkyl, polyhaloalkyl, and perhaloalkyl.

The term “alkyl” refers to a saturated linear or branched-chain monovalent hydrocarbon radical. In one example, the alkyl radical is one to eighteen carbon atoms (C₁₋₁₈). In other examples, the alkyl radical is C₁₋₁₂, C₁₋₁₀, C₁₋₈, C₁₋₆, C₁₋₅, C₁₋₄, or C₁₋₃. Examples of alkyl groups include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl and 1-octyl.

The term “oxo” refers to =O.

The term “alkoxy” refers to —O-alkyl.

The terms “cyano” or “nitrile” refers to —C≡N or —CN.

The term “haloalkoxy” refers to —O-haloalkyl.

The terms “hydroxy” and “hydroxyl” refer to —OH.

The term “alkylidene” refers to linear or branched-chain monovalent hydrocarbon radical having formula ═CR′R″, where R‘ and R’ can be the same or different. In one example, an alkylidene radical is 1 to 6 carbons (C₁₋₆). In another example, the alkylidene radical is C₁₋₃, C₁₋₂, or C₁. Examplary alkylidenes include, but are not limited to, methylidene (=CH₂), ethylidene (=CHCH₃), and propylidene (=CH—CH₂—CH₃).

The term “alkenyl” refers to linear or branched-chain monovalent hydrocarbon radical with at least one carbon-carbon double bond, and includes radicals having “cis” and “trans” orientations, or alternatively, “E” and “Z” orientations. In one example, the alkenyl radical is two to eighteen carbon atoms (C₂₋₁₈). In other examples, the alkenyl radical is C₂₋₁₂, C₂₋₁₀, C₂₋₈, C₂₋₆, or C₂₋₃. Examples include, but are not limited to, ethenyl or vinyl (—CH═CH₂), prop-1-enyl (—CH═CHCH₃), prop-2-enyl (—CH₂CH═CH₂), 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, and hexa-1,3-dienyl.

The term “alkynyl” refers to a linear or branched monovalent hydrocarbon radical with at least one carbon-carbon, triple bond. In one example, the alkynyl radical is two to eighteen carbon atoms (C₂₋₁₈). In other examples, the alkynyl radical is C₂₋₁₂, C₂₋₁₀, C₂₋₈, C₂₋₆, or C₂₋₃. Examples include, but are not limited to, ethynyl (—C≡CH), prop-1-ynyl (—C≡CCH₃), prop-2-ynyl (propargyl, —CH₂C≡CH), but-1-ynyl, but-2-ynyl, and but-3-ynyl.

The term “alkylene” refers to a saturated, branched, or straight chain hydrocarbon group having two monovalent radical centers derived by the removal of two hydrogen atoms from the same or two different carbon atoms of a parent alkane. In one example, the divalent alkylene group is one to eighteen carbon atoms (C₁₋₁₈). In other examples, the divalent alkylene group is C₁₋₁₂, C₁₋₁₀, C₁₋₈, C₁₋₆, C₁₋₅, C₁₋₄, or C₁₋₃. Example alkylene groups include methylene (—CH₂—), 1,1-ethyl (—CH(CH₃)—), (1,2-ethyl (—CH₂CH₂—), 1,1-propyl (—CH(CH₂CH₃)—), 2,2-propyl (—C(CH₃)₂—), 1,2-propyl (—CH(CH₃)CH₂—), 1,3-propyl (—CH₂CH₂CH₂—), 1,1-dimethyleth-1,2-yl (—C(CH₃)₂CH₂—), 1,4-butyl (—CH₂CH₂CH₂CH₂—), and the like.

The term “cycloalkyl” refers to a saturated hydrocarbon ring group. Cycloalkyl encompasses mono-, bi-, tricyclic, spiro and bridged, saturated ring systems. In one example, the cycloalkyl group is 3 to 12 carbon atoms (C₃₋₁₂). In other examples, cycloalkyl is C₃₋₄, C₃₋₅, C₃₋₇, C₃₋₈, C₃₋₁₀, or C₅₋₁₀. In other examples, the cycloalkyl group, as a monocycle, is C₃₋₄, C₃₋₈, C₃₋₆, or C₅₋₆. In another example, the cycloalkyl group, as a bicycle, is C₇-C₁₂. In another example, the cycloalkyl group, as a spiro system, is C₅₋₁₂. Examples of monocyclic cycloalkyl include cyclopropyl, cyclobutyl, cyclopentyl, cyclohexyl, cycloheptyl, cyclooctyl, cyclononyl, cyclodecyl, cycloundecyl and cyclododecyl. Exemplary arrangements of bicyclic cycloalkyls having 7 to 12 ring atoms include, but are not limited to, [4,4], [4,5], [5,5], [5,6] or [6,6] ring systems. Exemplary bridged bicyclic cycloalkyls include, but are not limited to, bicyclo[2.2.1]heptane, bicyclo[2.2.2]octane and bicyclo[3.2.2]nonane. Examples of spirocycloalkyl include, spiro[2.2]pentane, spiro[2.3]hexane, spiro[2.4]heptane, spiro[2.5]octane and spiro[4.5]decane.

The terms “heterocyclic group”, “heterocyclic”, “heterocycle”, “heterocyclyl”, or “heterocyclo” are used interchangeably and refer to any mono-, bi-, tricyclic, spiro or bridged, saturated, partially saturated or unsaturated, non-aromatic ring system, having 3 to 20 ring atoms, where the ring atoms are carbon, and at least one atom in the ring or ring system is a heteroatom selected from nitrogen, sulfur or oxygen. If any ring atom of a cyclic system is a heteroatom, that system is a heterocycle, regardless of the point of attachment of the cyclic system to the rest of the molecule. In one example, heterocyclyl includes 3-10 ring atoms (“members”) and includes monocycles, bicycles, tricycles, spiro, and bridged ring systems, wherein the ring atoms are carbon, where at least one atom in the ring or ring system is a heteroatom selected from nitrogen, sulfur or oxygen. In other examples, heterocyclyl includes 4-10 or 5-10 ring atoms. In one example, heterocyclyl includes 1 to 4 heteroatoms. In one example, heterocyclyl includes 1 to 3 heteroatoms. In another example, heterocyclyl includes 3- to 7-membered monocycles having 1-2, 1-3 or 1-4 heteroatoms selected from nitrogen, sulfur or oxygen. In another example, heterocyclyl includes 4- to 6-membered monocycles having 1-2, 1-3 or 1-4 heteroatoms selected from nitrogen, sulfur or oxygen. In another example, heterocyclyl includes 3-membered monocycles. In another example, heterocyclyl includes 4-membered monocycles. In another example, heterocyclyl includes 5-6 membered monocycles. In some embodiments, a heterocycloalkyl includes at least one nitrogen. In one example, the heterocyclyl group includes 0 to 3 double bonds. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Example heterocycles are oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydrofuranyl, dihydrothienyl, tetrahydrothienyl, imidazolidinyl, piperidinyl, piperazinyl, isoquinolinyl, tetrahydroisoquinolinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, 1,1-dioxoisothiazolyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, thiazinyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-onyl, azaspiro[5.5]undecanyl, tetrahydroindolyl, octahydroindolyl, tetrahydroisoindolyl, tetrahydroindazolyl, 1,1-dioxohexahydrothiopyranyl.

In particular embodiments, a heterocyclyl group or a heteroaryl group is attached at a carbon atom of the heterocyclyl group or the heteroaryl group. By way of example, carbon bonded heterocyclyl groups include bonding arrangements at position 2, 3, 4, 5, or 6 of a pyridine ring, position 3, 4, 5, or 6 of a pyridazine ring, position 2, 4, 5, or 6 of a pyrimidine ring, position 2, 3, 5, or 6 of a pyrazine ring, position 2, 3, 4, or 5 of a furan, tetrahydrofuran, thiofuran, thiophene, pyrrole or tetrahydropyrrole ring, position 2, 4, or 5 of an oxazole, imidazole or thiazole ring, position 3, 4, or 5 of an isoxazole, pyrazole, or isothiazole ring, position 2 or 3 of an aziridine ring, position 2, 3, or 4 of an azetidine ring, position 2, 3, 4, 5, 6, 7, or 8 of a quinoline ring or position 1, 3, 4, 5, 6, 7, or 8 of an isoquinoline ring.

In certain embodiments, the heterocyclyl group or heteroaryl group is N-attached. By way of example, nitrogen bonded heterocyclyl or heteroaryl groups include bonding arrangements at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or p-carboline.

“Fused” refers to any ring structure described herein that shares one or more atoms (e.g., carbon or nitrogen atoms) with an existing ring structure in the compounds described herein.

The term “acyl” refers to a carbonyl containing substituent represented by the formula —C(═O)—R in which R is a substituent such as hydrogen, alkyl, cycloalkyl, aryl or heterocyclyl, wherein the alkyl, cycloalkyl, aryl and heterocyclyl are as defined herein. Acyl groups include alkanoyl (e.g., acetyl), aroyl (e.g., benzoyl), and heteroaroyl (e.g., pyridinoyl).

The term “haloalkyl” refers to an alkyl chain in which one or more hydrogen has been replaced by a halogen. Examples of haloalkyls are trifluoromethyl, difluoromethyl, and fluoromethyl. A substituted haloalkyl refers to a haloalkyl having a moiety other than a halogen.

As used herein a wavy line “ [symbol] ” that intersects a bond in a chemical structure indicate the point of attachment of the atom to which the wavy bond is connected in the chemical structure to the remainder of a molecule, or to the remainder of a fragment of a molecule.

In certain embodiments, divalent groups are described generically without specific bonding configurations. It is understood that the generic description is meant to include both bonding configurations, unless specified otherwise. For example, in the group R¹—R²—R³, if the group R² is described as —CH₂C(O)—, then it is understood that this group can be bonded both as R¹—CH₂C(O)—R³, and as R¹—C(O)CH₂—R³, unless specified otherwise.

The term “pharmaceutically acceptable” refers to molecular entities and compositions that do not produce an adverse, allergic or other untoward reaction when administered to an animal, such as, for example, a human, as appropriate.

Compounds described herein may be in the form of a salt, such as a pharmaceutically acceptable salt. “Pharmaceutically acceptable salts” include both acid and base addition salts. “Pharmaceutically acceptable acid addition salt” refers to those salts which retain the biological effectiveness and properties of the free bases and which are not biologically or otherwise undesirable, formed with inorganic acids such as hydrochloric acid, hydrobromic acid, sulfuric acid, nitric acid, carbonic acid, phosphoric acid and the like, and organic acids may be selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, malonic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, benzenesulfonic acid, p-toluenesulfonic acid, salicylic acid and the like.

The term “pharmaceutically acceptable base addition salts” include those derived from inorganic bases such as sodium, potassium, lithium, ammonium, calcium, magnesium, iron, zinc, copper, manganese, aluminum salts and the like. Particular base addition salts are the ammonium, potassium, sodium, calcium and magnesium salts. Salts derived from pharmaceutically acceptable organic nontoxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, tromethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particular organic non-toxic bases include isopropylamine, diethylamine, ethanolamine, tromethamine, dicyclohexylamine, choline, and caffeine.

In some embodiments, a salt is selected from a hydrochloride, hydrobromide, trifluoroacetate, sulfate, phosphate, acetate, fumarate, maleate, tartrate, lactate, citrate, pyruvate, succinate, oxalate, methanesulfonate, p-toluenesulfonate, bisulfate, benzenesulfonate, ethanesulfonate, malonate, xinafoate, ascorbate, oleate, nicotinate, saccharinate, adipate, formate, glycolate, palmitate, L-lactate, D-lactate, aspartate, malate, L-tartrate, D-tartrate, stearate, furoate (e.g., 2-furoate or 3-furoate), napadisylate (naphthalene-1,5-disulfonate or naphthalene-1-(sulfonic acid)-5-sulfonate), edisylate (ethane-1,2-disulfonate or ethane-1-(sulfonic acid)-2-sulfonate), isothionate (2-hydroxyethylsulfonate), 2-mesitylenesulfonate, 2-naphthalenesulfonate, 2,5-dichlorobenzenesulfonate, D-mandelate, L-mandelate, cinnamate, benzoate, adipate, esylate, malonate, mesitylate (2-mesitylenesulfonate), napsylate (2-naphthalenesulfonate), camsylate (camphor-10-sulfonate, for example (1S)-(+)-10-camphorsulfonic acid salt), glutamate, glutarate, hippurate (2-(benzoylamino)acetate), orotate, xylate (p-xylene-2-sulfonate), and pamoic (2,2′-dihydroxy-1,1′-dinaphthylmethane-3,3′-dicarboxylate).

A “sterile” formulation is aseptic or free from all living microorganisms and their spores.

The term “stereoisomers” refer to compounds that have identical chemical constitution, but differ with regard to the arrangement of the atoms or groups in space. Stereoisomers include diastereomers, enantiomers, atropisomers, conformers and the like.

The term “chiral” refers to molecules that have the property of non-superimposability of the mirror image partner, while the term “achiral” refers to molecules which are superimposable on their mirror image partner.

The term “diastereomer” refers to a stereoisomer with two or more centers of chirality and whose molecules are not mirror images of one another. Diastereomers have different physical properties, e.g., melting points, boiling points, spectral properties or biological activities. Mixtures of diastereomers may separate under high resolution analytical procedures such as electrophoresis and chromatography such as HPLC.

The term “enantiomers” refers to two stereoisomers of a compound that are non-superimposable mirror images of one another.

The term “atropisomers” refers to two conformers resulting from hindered rotation about a single bond where the steric strain barrier to rotation can be high enough to allow for the isolation of the each conformer.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The term “tautomer” or “tautomeric form” refers to structural isomers of different energies that are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

Certain compounds described herein can exist in unsolvated forms as well as solvated forms, including hydrated forms. A “solvate” refers to an association or complex of one or more solvent molecules and a compound described herein. Examples of solvents that form solvates include water, isopropanol, ethanol, methanol, DMSO, ethyl acetate, acetic acid, and ethanolamine. Certain compounds described herein can exist in multiple crystalline or amorphous forms. In general, all physical forms are contemplated herein. The term “hydrate” refers to the complex where the solvent molecule is water.

The compounds and pharmaceutically acceptable salts thereof described herein also embrace isotopically-labeled compounds that are identical to those recited herein, but for the fact that one or more atoms are replaced by an atom having an atomic mass or mass number different from the atomic mass or mass number usually found in nature. All isotopes of any particular atom or element as specified are contemplated herein, and their uses. Exemplary isotopes that can be incorporated into compounds and pharmaceutically acceptable salts thereof described herein include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I. Certain isotopically-labeled compounds or pharmaceutical acceptable salts thereof described herein (e.g., those labeled with ³H and ¹⁴C) are useful in compound and/or substrate tissue distribution assays. Tritiated (³H) and carbon-14 (¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labeled compounds or pharmaceutical acceptable salts thereof described herein can generally be prepared by following procedures analogous to those disclosed in the Examples herein below, by substituting an isotopically labeled reagent for a non-isotopically labeled reagent.

The term “amino-protecting group” as used herein refers to a derivative of the groups commonly employed to block or protect an amino group while reactions are carried out on other functional groups on the compound. Examples of such protecting groups include carbamates, amides, alkyl and aryl groups, and imines, as well as many N-heteroatom derivatives that can be removed to regenerate the desired amine group. Particular amino protecting groups are Pmb (p-methoxybenzyl), Boc (tert-butyloxycarbonyl), Fmoc (9-fluorenylmethyloxycarbonyl) and Cbz (carbobenzyloxy). Further examples of these groups are found in T. W. Greene and P. G. M. Wuts, “Protecting Groups in Organic Synthesis, 3^{rd} ed., John Wiley & Sons, Inc., 1999. The term “protected amino” refers to an amino group substituted with one of the above amino-protecting groups.

The term “carboxy-protecting group” as used herein refers to those groups that are stable to the conditions of subsequent reaction(s) at other positions of the molecule, which may be removed at the appropriate point without disrupting the remainder of the molecule, to give the unprotected carboxy-group. Examples of carboxy protecting groups include, ester groups and heterocyclyl groups. Ester derivatives of the carboxylic acid group may be employed to block or protect the carboxylic acid group while reactions are carried out on other functional groups on the compound. Examples of such ester groups include substituted arylalkyl, including substituted benzyls, such as 4-nitrobenzyl, 4-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′-dimethoxybenzhydryl, 2,2′,4,4′-tetramethoxybenzhydryl, alkyl or substituted alkyl esters such as methyl, ethyl, t-butyl allyl or t-amyl, triphenylmethyl (trityl), 4-methoxytrityl, 4,4′-dimethoxytrityl, 4,4′,4″-trimethoxytrityl, 2-phenylprop-2-yl, thioesters such as t-butyl thioester, silyl esters such as trimethylsilyl, t-butyldimethylsilyl esters, phenacyl, 2,2,2-trichloroethyl, beta-(trimethylsilyl)ethyl, beta-(di(n-butyl)methylsilyl)ethyl, p-toluenesulfonylethyl, 4-nitrobenzylsulfonylethyl, allyl, cinnamyl, 1-(trimethylsilylmethyl)prop-1-en-3-yl, and like moieties. Another example of carboxy-protecting groups are heterocyclyl groups such as 1,3-oxazolinyl. Further examples of these groups are found in T. W. Greene and P. G. M. Wuts, “Protecting Groups in Organic Synthesis, 3^{rd} ed., John Wiley & Sons, Inc., 1999. The term “protected carboxy” refers to a carboxy group substituted with one of the above carboxy-protecting groups.

Compounds and pharmaceutically acceptable salts thereof described herein may contain one or more asymmetric carbon atoms. Accordingly, the compounds may exist as diastereomers, enantiomers or mixtures thereof. The syntheses of the compounds may employ racemates, diastereomers or enantiomers as starting materials or as intermediates. Mixtures of particular diastereomeric compounds may be separated, or enriched in one or more particular diastereomers, by chromatographic or crystallization methods. Similarly, enantiomeric mixtures may be separated, or enantiomerically enriched, using the same techniques or others known in the art. Each of the asymmetric carbon or nitrogen atoms may be in the R or S configuration and both of these configurations are contemplated herein.

In the structures shown herein, where the stereochemistry of any particular chiral atom is not specified, then all stereoisomers are contemplated and included. Where stereochemistry is specified by a solid wedge or dashed line representing a particular configuration, then that stereoisomer is so specified and defined. Unless otherwise specified, if solid wedges or dashed lines are used, relative stereochemistry is intended.

A “subject,” “individual,” or “patient” is a vertebrate and are used interchangeably herein. In certain embodiments, the vertebrate is a mammal. Mammals include, but are not limited to, farm animals (such as cows), sport animals, pets (such as guinea pigs, cats, dogs, rabbits and horses), primates, mice and rats. In certain embodiments, a mammal is a human. In embodiments comprising administration of a compound of to a patient, the patient is typically in need thereof.

The terms “inhibiting” and “reducing,” or any variation of these terms, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of activity compared to normal.

The term “treatment” refers to clinical intervention designed to alter the natural course of the patient or cell being treated during the course of clinical pathology. Desirable effects of treatment include decreasing the rate of disease progression, ameliorating or palliating the disease state, and remission or improved prognosis. For example, a patient is successfully “treated” if one or more symptoms associated with a cancer described herein are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of patients.

The term “delaying progression” of a disease refers to deferring, hindering, slowing, retarding, stabilizing, and/or postponing development of a cancer described herein. This delay can be of varying lengths of time, depending on the history of the cancer and/or patient being treated. As is evident to one skilled in the art, a sufficient or significant delay can, in effect, encompass prevention, in that the patient does not develop cancer or relapse.

A “mutant KRas mediated disease” and the like refer to a disease described herein (e.g. a cancer described herein) having symptoms or requiring treatment as set forth herein that is/are wholly or partly associated with, a result of, a function of, or otherwise correlated to mutant KRas activity as described herein. In one such embodiment, the mutant KRas is KRas^{G12D}.

An “effective amount” or “therapeutically effective amount” is at least the minimum amount required to effect a measurable improvement or prevention of a cancer described herein. An effective amount herein may vary according to factors such as the disease state, age, sex, and weight of the patient, and the ability of the agent to elicit a desired response in the patient. An effective amount is also one in which any toxic or detrimental effects of the treatment are outweighed by the therapeutically beneficial effects. Beneficial or desired results include results such as eliminating or reducing the risk, lessening the severity, delaying the onset of the disease (including biochemical, histological and/or behavioral symptoms of the disease, its complications and intermediate pathological phenotypes presenting during development of the disease), decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In some embodiments, an effective amount of the drug may have the effect in reducing the number of cancer cells; reducing the tumor size; inhibiting (i.e., slow or stop) cancer cell infiltration into peripheral organs; inhibit (i.e., slow or stop) tumor metastasis; inhibiting (i.e., slow or stop) tumor growth; and/or relieving one or more of the symptoms associated with the disorder. An effective amount can be administered in one or more administrations.

An “administration period” or “cycle” refers to a period of time comprising administration of one or more compounds or pharmaceutically acceptable salts thereof described herein or an additional therapeutic agent (i.e. a chemotherapeutic agent) and an optional period of time comprising no administration of one or more of agents or compounds described herein. A “rest period” refers to a period of time where at least one of agent or compound described herein is not administered. In one embodiment, a rest period refers to a period of time where no agent or compound described herein is administered. A rest period as provided herein can in some instances include administration of an additional agent in the absence of a compound or pharmaceutically acceptable salt thereof described herein or vice versa. In such instances, administration of any agent during a rest period should not interfere or detriment administration of a compound or pharmaceutically acceptable salt thereof described herein.

A “dosing regimen” refers to a period of administration of a compound or pharmaceutically acceptable salt thereof described herein comprising one or more cycles, where each cycle can include administration of a compound or pharmaceutically acceptable salt thereof described herein at different times or in different amounts.

“QD” refers to administration of a compound or pharmaceutically acceptable salt thereof once daily.

“BID” refers to administration of a compound or pharmaceutically acceptable salt thereof twice a day.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times (i.e. sequential administration) in separate compositions, or administration in a composition in which both agents are present.

A “1 L therapy” refers to the first line therapy administered to a treatment naïve cancer patient. Likewise, a 2 L, 3 L, and the like refer to subsequent therapies administered to a patient.

The term “package insert” is used to refer to instructions customarily included in commercial packages of therapeutic products, that contain information about the indications, usage, dosage, administration, contraindications and/or warnings concerning the use of such therapeutic products.

The terms “antagonist” and “inhibitor” are used interchangeably, and they refer to a compound having the ability to inhibit a biological function of a target protein, whether by inhibiting the activity or expression of the protein, such as a mutant form of KRas. Accordingly, the terms “antagonist” and “inhibitors” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition. A preferred biological activity inhibited by an antagonist is associated with the development, growth, or spread of a tumor.

The term “agonist” as used herein refers to a compound having the ability to initiate or enhance a biological function of a target protein, whether by inhibiting the activity or expression of the target protein. Accordingly, the term “agonist” is defined in the context of the biological role of the target polypeptide. While preferred agonists herein specifically interact with (e.g., bind to) the target, compounds that initiate or enhance a biological activity of the target polypeptide by interacting with other members of the signal transduction pathway of which the target polypeptide is a member are also specifically included within this definition.

The terms “cancer” and “cancerous”, “neoplasm”, and “tumor” and related terms are used interchangeably herein and refer to or describe the physiological condition in mammals that is typically characterized by unregulated cell growth. A “tumor” comprises one or more cancerous cells. Examples of cancer include carcinoma, blastoma, sarcoma, seminoma, glioblastoma, melanoma, leukemia, and myeloid or lymphoid malignancies. More particular examples of such cancers include squamous cell cancer (e.g., epithelial squamous cell cancer) and lung cancer including small-cell lung cancer, non-small cell lung cancer (“NSCLC”), adenocarcinoma of the lung and squamous carcinoma of the lung. Other cancers include skin, keratoacanthoma, follicular carcinoma, hairy cell leukemia, buccal cavity, pharynx (oral), lip, tongue, mouth, salivary gland, esophageal, larynx, hepatocellular, gastric, stomach, gastrointestinal, small intestine, large intestine, pancreatic, cervical, ovarian, liver, bladder, hepatoma, breast, colon, rectal, colorectal, genitourinary, biliary passage, thyroid, papillary, hepatic, endometrial, uterine, salivary gland, kidney or renal, prostate, testis, vulval, peritoneum, anal, penile, bone, multiple myeloma, B-cell lymphoma, diffuse large B-Cell lymphoma (DLBCL), central nervous system, brain, head and neck, Hodgkin's, and associated metastases. Other examples of neoplastic disorders include myeloproliferative disorders, such as polycythemia vera, essential thrombocytosis, myelofibrosis, such as primary myelofibrosis, and chronic myelogenous leukemia (CML).

A “chemotherapeutic agent” is an agent useful in the treatment of a given disorder, for example, cancer or inflammatory disorders. Examples of chemotherapeutic agents are well-known in the art. Additionally, chemotherapeutic agents include pharmaceutically acceptable salts, acids or derivatives of any of chemotherapeutic agents, as well as combinations of two or more of them.

Unless otherwise stated, structures depicted herein are also meant to include compounds that differ only in the presence of one or more isotopically enriched atoms. Exemplary isotopes that can be incorporated into compounds and pharmaceutically acceptable salts thereof described herein, include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, in compounds and pharmaceutically acceptable salts thereof described herein, one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Isotopically labeled compounds can generally be prepared by following procedures analogous to those disclosed in the Schemes or in the Examples herein, by substituting an isotopically labeled reagent for a non-isotopically labeled reagent.

It is specifically contemplated that any limitation discussed with respect to one embodiment provided herein may apply to any other embodiment provided herein. Furthermore, any compound and pharmaceutically acceptable salts thereof described herein or composition described herein may be used in any method provided herein, and any method provided herein may be used to produce or to utilize any compound and pharmaceutically acceptable salts thereof described herein or composition described herein.

Throughout this application, the term “about” is used to indicate that a value includes the standard deviation of error for the device or method being employed to determine the value.

### Compounds

Provided herein are compounds of formula (I*):

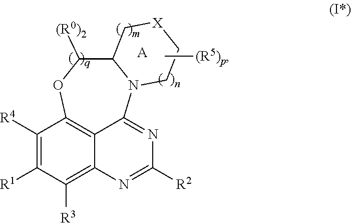

or a stereoisomer, atropisomer, tautomer, or pharmaceutically acceptable salt thereof, wherein;
X is O or NR⁶;
n is 1, 2, or 3;
m is 1, 2, or 3;
p is 0, 1, or 2;
q is 1 or 2;
wherein n and m together make a 6-, 7-, or 8-membered ring A;
each R⁰ is independently hydrogen or methyl;
R¹ is R⁷-substituted or unsubstituted napthyl, R⁷-substituted or unsubstituted isoquinolinyl, R⁷-substituted or unsubstituted indazolyl, R⁷-substituted or unsubstituted benzothiazolyl, R^{7A}-substituted or unsubstituted phenyl, or R^{7A}-substituted or unsubstituted pyridinyl;
each R⁷ is independently hydrogen, halogen, —OH, NH₂, N(Me)₂, unsubstituted C₁₋₃ alkyl, unsubstituted C₁₋₃ haloalkyl, or unsubstituted cyclopropyl;
each R^{7A} is independently hydrogen, halogen, NH₂, N(Me)₂, unsubstituted C₁₋₃ alkyl, unsubstituted C₁₋₃ haloalkyl, or unsubstituted cyclopropyl;
R² is hydrogen, L¹-O-L²-R⁸, R^{8A}-substituted or unsubstituted C₁₋₃ alkyl, or R^{8B}-substituted or unsubstituted 4-10 membered heterocycle;
   wherein when R² is hydrogen, R¹ is R⁷-substituted indazolyl, and n and m are 1, then p is not zero and R⁶ is not H;
L¹ is a bond or R^{L1}-substituted or unsubstituted C₁₋₃ alkylene;
R^{L1} is halogen or unsubstituted C₁₋₃ alkyl;
L² is a bond or unsubstituted C₁₋₃ alkylene;
R⁸ is R⁹-substituted or unsubstituted C₁₋₃ alkyl, R⁹-substituted or unsubstituted 4-10 membered heterocycle comprising N, S, or O;
each R⁹ is independently halogen, oxo, —OCF₃, —OCHF₂, —OCH₂F, unsubstituted C₁₋₃ alkyl, unsubstituted C₁₋₃ haloalkyl, unsubstituted C₁₋₃ alkoxy, R¹⁰-substituted or unsubstituted C₁₋₃ alkylidene, or R¹⁰-substituted or unsubstituted C₃₋₄ cycloalkyl, or R¹⁰-substituted or unsubstituted 3 or 4-membered heterocycle; or wherein
   two R⁹ together form a R¹⁰-substituted or unsubstituted C₃₋₅ cycloalkyl or a R¹⁰-substituted or unsubstituted C₃₋₅ heterocycle comprising one or more oxygen atoms;
R¹⁰ is hydrogen, halogen, or unsubstituted C₁₋₃ alkyl;
each R^{8A} is independently R^{9A}-substituted or unsubstituted C₁₋₃ alkyl, R^{9A}-substituted or unsubstituted C₁₋₃ alkoxy, R^{9A}-substituted or unsubstituted C₃₋₄ cycloalkyl, or R^{9A}-substituted or unsubstituted 4-6 membered heterocycle;
each R^{9A} is independently halogen, oxo, unsubstituted C₁₋₃ alkyl, unsubstituted C₁₋₃ haloalkyl, unsubstituted C₁₋₃ alkoxy, unsubstituted C₁₋₃ alkylidene, R⁹-substituted or unsubstituted C₃₋₄ cycloalkyl, or R⁹-substituted or unsubstituted 4-10 membered heterocycle comprising N, S, or O;
R^{8B} is independently halogen, oxo, —NH₂, unsubstituted C₁₋₃ alkyl, unsubstituted C₁₋₃ haloalkyl, unsubstituted C₁₋₃ alkoxy, or unsubstituted C₁₋₃ alkylidene;
R³ and R⁴ are each independently hydrogen, —CN, halogen, unsubstituted C₁₋₃ alkyl, or unsubstituted cyclopropyl;
each R⁵ is independently halogen, oxo, unsubstituted C₁₋₃ alkyl, or unsubstituted C₁₋₃ haloalkyl; or wherein;
   two R⁵ together form a bridge between two carbon atoms of ring A, wherein the bridge comprises 1-3 carbons and optionally one heteroatom selected from O and N; or
   two R⁵ together form a bridge between two carbon atoms of ring A, wherein the bridge comprises one of O or NR¹¹;
R¹¹ is hydrogen, C(O)CH₃, or unsubstituted C₁₋₃ alkyl;
R⁶ is hydrogen or R^{6A}-substituted or unsubstituted C₁₋₆ alkyl, R^{6A}-substituted or unsubstituted C₁₋₆ haloalkyl, R^{6A}-substituted or unsubstituted C₁₋₆ alkenyl; R^{6A}-substituted or unsubstituted C₁₋₆ alkynyl, or R^{6A}-substituted or unsubstituted 3-4 membered heterocycle;
R^{6A} is halogen, CN, OR^{6B}, SR^{6C}, S(O)₂R^{6C}, C(O)R^{6B}, unsubstituted C₁₋₃ alkyl; or R^{6B}-substituted or unsubstituted 3-4 membered heterocycle; and
R^{6B} and R^{6C} are each independently C₁₋₃ alkyl or C₁₋₃ haloalkyl.

In one embodiment, X, p, R¹, R², R³, R⁴, R⁵, R⁶, R^{6A}, R^{6B}, R^{6C}, R⁷, R^{7A}, R⁸, R^{8A}, R^{8B}, R⁹, R^{9A}, R¹⁰, and R¹¹ are as described herein, q is 1, and n and m are independently 1 or 2 wherein n and m together make a 6- or 7-membered ring A.

In one embodiment, q is 1.

Provided herein are compounds of formula (I):

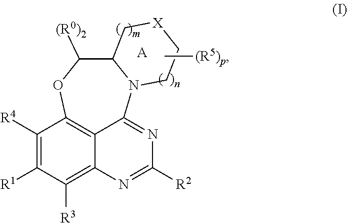

or a stereoisomer, atropisomer, tautomer, or pharmaceutically acceptable salt thereof, wherein;
X is O or NR⁶;
n is 1, 2, or 3;
m is 1, 2, or 3;
p is 0, 1, or 2;
wherein n and m together make a 6-, 7-, or 8-membered ring A;
each R⁰ is independently hydrogen or methyl;
R¹ is R⁷-substituted or unsubstituted napthyl, R⁷-substituted or unsubstituted isoquinolinyl, R⁷-substituted or unsubstituted indazolyl, R⁷-substituted or unsubstituted benzothiazolyl, R^{7A}-substituted or unsubstituted phenyl, or R^{7A}-substituted or unsubstituted pyridinyl;
each R⁷ is independently hydrogen, halogen, —OH, NH₂, N(Me)₂, unsubstituted C₁₋₃ alkyl, unsubstituted C₁₋₃ haloalkyl, or unsubstituted cyclopropyl;
each R^{7A} is independently hydrogen, halogen, NH₂, N(Me)₂, unsubstituted C₁₋₃ alkyl, unsubstituted C₁₋₃ haloalkyl, or unsubstituted cyclopropyl;
R² is hydrogen, L¹-O-L²-R⁸, R^{8A}-substituted or unsubstituted C₁₋₃ alkyl, or R^{8B}-substituted or unsubstituted 4-10 membered heterocycle;
   wherein when R² is hydrogen, R¹ is R⁷-substituted indazolyl, and n and m are 1, then p is not zero and R⁶ is not H;
L¹ is a bond or R^{L1}-substituted or unsubstituted C₁₋₃ alkylene;
R^{L1} is halogen or unsubstituted C₁₋₃ alkyl;
L² is a bond or unsubstituted C₁₋₃ alkylene;
R⁸ is R⁹-substituted or unsubstituted C₁₋₃ alkyl, R⁹-substituted or unsubstituted 4-10 membered heterocycle comprising N, S, or O;
each R⁹ is independently halogen, oxo, —OCF₃, —OCHF₂, —OCH₂F, unsubstituted C₁₋₃ alkyl, unsubstituted C₁₋₃ haloalkyl, unsubstituted C₁₋₃ alkoxy, R¹⁰-substituted or unsubstituted C₁₋₃ alkylidene, or R¹⁰-substituted or unsubstituted C₃₋₄ cycloalkyl, or R¹⁰-substituted or unsubstituted 3 or 4-membered heterocycle; or wherein
   two R⁹ together form a R¹⁰-substituted or unsubstituted C₃₋₅ cycloalkyl or a R¹⁰-substituted or unsubstituted C₃₋₅ heterocycle comprising one or more oxygen atoms;
R¹⁰ is hydrogen, halogen, or unsubstituted C₁₋₃ alkyl;
each R^{8A} is independently R^{9A}-substituted or unsubstituted C₁₋₃ alkyl, R^{9A}-substituted or unsubstituted C₁₋₃ alkoxy, R^{9A}-substituted or unsubstituted C₃₋₄ cycloalkyl, or R^{9A}-substituted or unsubstituted 4-6 membered heterocycle;
each R^{9A} is independently halogen, oxo, unsubstituted C₁₋₃ alkyl, unsubstituted C₁₋₃ haloalkyl, unsubstituted C₁₋₃ alkoxy, unsubstituted C₁₋₃ alkylidene, R⁹-substituted or unsubstituted C₃₋₄ cycloalkyl, or R⁹-substituted or unsubstituted 4-10 membered heterocycle comprising N, S, or O;
R^{8B} is independently halogen, oxo, —NH₂, unsubstituted C₁₋₃ alkyl, unsubstituted C₁₋₃ haloalkyl, unsubstituted C₁₋₃ alkoxy, or unsubstituted C₁₋₃ alkylidene;
R³ and R⁴ are each independently hydrogen, —CN, halogen, unsubstituted C₁₋₃ alkyl, or unsubstituted cyclopropyl;
each R⁵ is independently halogen, oxo, unsubstituted C₁₋₃ alkyl, or unsubstituted C₁₋₃ haloalkyl; or wherein;
   two R⁵ together form a bridge between two carbon atoms of ring A, wherein the bridge comprises 1-3 carbons and optionally one heteroatom selected from O and N; or
   two R⁵ together form a bridge between two carbon atoms of ring A, wherein the bridge comprises one of O or NR¹¹;
R¹¹ is hydrogen, C(O)CH₃, or unsubstituted C₁₋₃ alkyl;
R⁶ is hydrogen or R^{6A}-substituted or unsubstituted C₁₋₆ alkyl, R^{6A}-substituted or unsubstituted C₁₋₆ haloalkyl, R^{6A}-substituted or unsubstituted C₁₋₆ alkenyl; R^{6A}-substituted or unsubstituted C₁₋₆ alkynyl, or R^{6A}-substituted or unsubstituted 3-4 membered heterocycle;
R^{6A} is halogen, CN, OR^{6B}, SR^{6C}, S(O)₂R^{6C}, C(O)R^{6B}, unsubstituted C₁₋₃ alkyl; or R^{6B}-substituted or unsubstituted 3-4 membered heterocycle; and
R^{6B} and R^{6C} are each independently C₁₋₃ alkyl or C₁₋₃ haloalkyl.

In one embodiment, X, p, R¹, R², R³, R⁴, R⁵, R⁶, R^{6A}, R^{6B}, R^{6C}, R⁷, R^{7A}, R⁸, R^{8A}, R^{8B}, R⁹, R^{9A}, R¹⁰, and R¹¹ are as described herein and n and m are independently 1 or 2 wherein n and m together make a 6- or 7-membered ring A.

In one embodiment, each R⁰ is hydrogen such that the compound of formula (I) has formula:

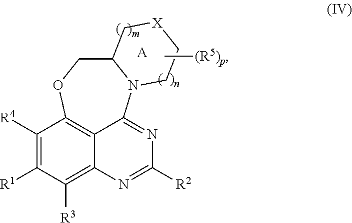

wherein X, m, n, p, R¹, R², R³, R⁴, R⁵, R⁶, R^{6A}, R^{6B}, R^{6C}, R⁷, R^{7A}, R⁸, R^{8A}, R^{8B}, R⁹, R^{9A}, R¹⁰, and R¹¹ are as described herein for formula (I).

In one embodiment, one R⁰ is hydrogen and one R⁰ is methyl such that the compound of formula (I) has formula:

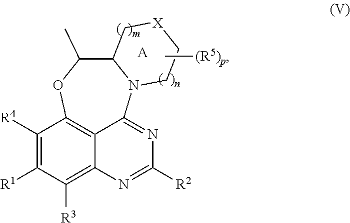

wherein X, m, n, p, R¹, R², R³, R⁴, R⁵, R⁶, R^{6A}, R^{6B}, R^{6C}, R⁷, R^{7A}, R⁸, R^{8A}, R^{8B}, R⁹, R^{9A}, R¹⁰, and R¹¹ are as described herein for formula (I).

In one embodiment of the compounds or a pharmaceutically acceptable salt thereof described herein of formula (V), the compound of formula (V) has formula:

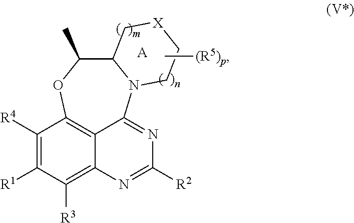

wherein X, m, n, p, R¹, R², R³, R⁴, R⁵, R⁶, R^{6A}, R^{6B}, R^{6C}, R⁷, R^{7A}, R⁸, R^{8A}, R^{8B}, R⁹, R^{9A}, R¹⁰, and R¹¹ are as described herein.

In one embodiment, R¹ is R⁷-substituted or unsubstituted napthyl, R⁷-substituted or unsubstituted indazolyl, R⁷-substituted or unsubstituted benzothiazolyl, R^{7A}-substituted or unsubstituted phenyl, or R^{7A}-substituted or unsubstituted pyridinyl. In another embodiment, R¹ is R⁷-substituted or unsubstituted napthyl, R⁷-substituted or unsubstituted indazolyl, R^{7A}-substituted or unsubstituted phenyl, or R^{7A}-substituted or unsubstituted pyridinyl. In still another embodiment, R¹ is R⁷-substituted or unsubstituted napthyl, R⁷-substituted or unsubstituted indazolyl, or R⁷-substituted or unsubstituted benzothiazolyl. In still another embodiment, R¹ is R⁷-substituted or unsubstituted napthyl or R⁷-substituted or unsubstituted indazolyl. In another embodiment, R¹ is R^{7A}-substituted or unsubstituted phenyl, or R^{7A}-substituted or unsubstituted pyridinyl. In another embodiment, R¹ is R⁷-substituted or unsubstituted phenyl, R⁷-substituted or unsubstituted indazolyl, or R⁷-substituted or unsubstituted pyridinyl.

In one such embodiment, R¹ is R⁷-substituted or unsubstituted phenyl. In another such embodiment, R¹ is R⁷-substituted or unsubstituted indazolyl. In another such embodiment, R¹ is R⁷-substituted or unsubstituted pyridinyl.

In one embodiment, R¹ has formula (A):

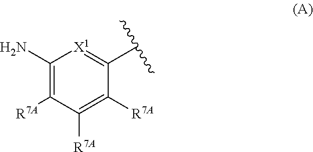

or a stereoisomer thereof, wherein X¹ is CH, N, or CF and R^{7A} is as described herein. In one such embodiment, X¹ is N or CF. In one such embodiment, R^{7A} is hydrogen, halogen, unsubstituted C₁₋₃ alkyl, or unsubstituted C₁₋₃ haloalkyl.

In one such embodiment, X¹ is N or CF and each R^{7A} is independently hydrogen, halogen, unsubstituted C₁₋₃ alkyl, or unsubstituted C₁₋₃ haloalkyl. In one such embodiment, R^{7A} is independently hydrogen, Cl, methyl, ethyl, or CF₃, where no more than one R^{7A} is hydrogen. In one embodiment, one R^{7A} is cyclopropyl.

In one such embodiment, the moiety of formula (A1) has formula:

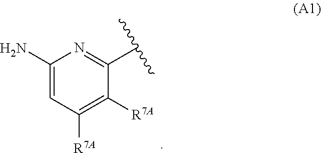

In one such embodiment, each R^{7A} is independently hydrogen, Cl, methyl, or CF₃. In another such embodiment, each R^{7A} is independently hydrogen, methyl, or CF₃.

In one such embodiment, R¹ is

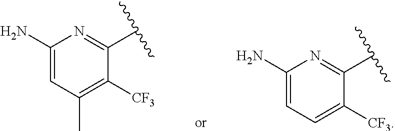 [SMILES: *c1nc(N)cc(C)c1C(F)(F)F ; *c1nc(N)ccc1C(F)(F)F]

In another such embodiment, R¹ is

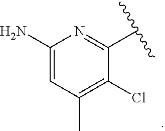 [SMILES: *c1nc(N)cc(C)c1Cl]

In one embodiment, R¹ is

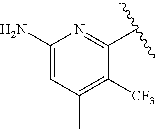 [SMILES: *c1nc(N)cc(C)c1C(F)(F)F]

In another embodiment, the moiety of formula (A) has formula:

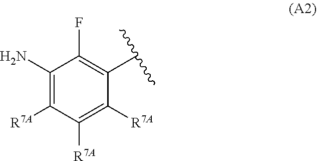

wherein each R^{7A} is independently hydrogen, halogen, unsubstituted C₁₋₃ alkyl or unsubstituted C₁₋₃ haloalkyl. In one such embodiment, each R^{7A} is independently hydrogen, F, methyl, ethyl, or CF₃. In such embodiments, no more than one R^{7A} is hydrogen. In another such embodiment, R^{7A} is not hydrogen.

In one such embodiment, R¹ is

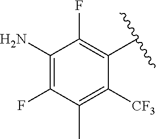 [SMILES: *c1c(F)c(N)c(F)c(C)c1C(F)(F)F]

In one such embodiment, R¹ is

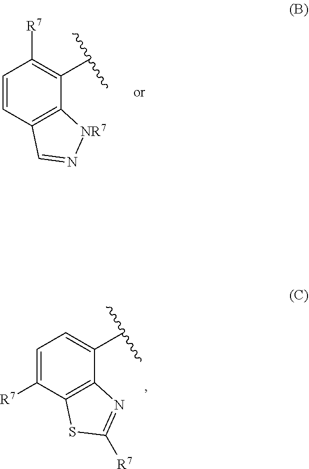

wherein each R⁷ is independently halogen, NH₂, N(Me)₂, or unsubstituted C₁₋₃ alkyl.

In one embodiment, R¹ is

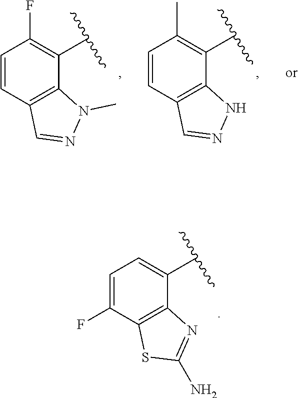

In another embodiment, R¹ is

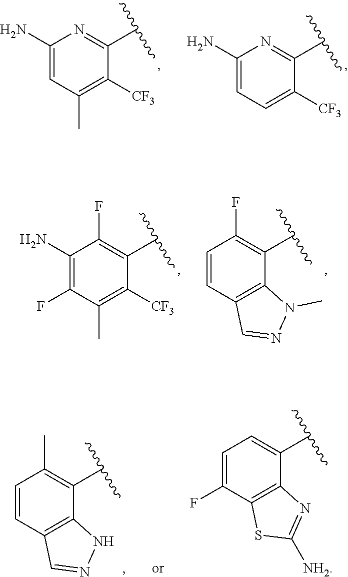 [SMILES: *c1nc(N)cc(C)c1C(F)(F)F ; *c1nc(N)ccc1C(F)(F)F ; *c1c(F)c(N)c(F)c(C)c1C(F)(F)F ; *c1c(F)ccc2cnn(C)c12 ; *c1c(C)ccc2cn[nH]c12 ; *c1ccc(F)c2sc(N)nc12]

In another embodiment, R¹ is:

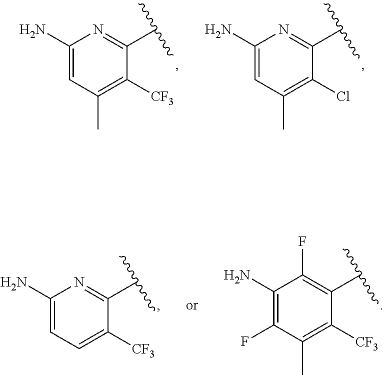

In another embodiment, R¹ is:

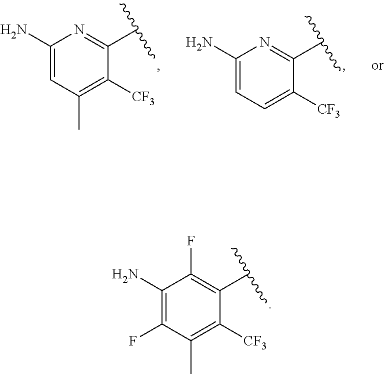 [SMILES: *c1nc(N)cc(C)c1C(F)(F)F ; *c1nc(N)ccc1C(F)(F)F ; *c1c(F)c(N)c(F)c(C)c1C(F)(F)F]

In one embodiment, R⁷ is independently hydrogen, halogen, —OH, NH₂, N(Me)₂, unsubstituted C₁₋₃ alkyl, unsubstituted C₁₋₃ haloalkyl. In another embodiment, R⁷ is independently halogen, NH₂, or unsubstituted C₁₋₃ alkyl, or unsubstituted C₁₋₃ haloalkyl. In one embodiment of the compounds or a stereoisomer, atropisomer, tautomer, or pharmaceutically acceptable salt thereof described herein, R⁷ is not —OH.

In one embodiment, R¹ is a moiety of formula (B) or (C) where R⁷ is independently hydrogen, halogen, or unsubstituted C₁₋₃ alkyl. In one such embodiment, R⁷ is independently hydrogen or unsubstituted C₁₋₃ alkyl (e.g. methyl). In another such embodiment, R⁷ is independently halogen (e.g. F) or unsubstituted C₁₋₃ alkyl (e.g. methyl).

In one embodiment, R¹ is a moiety of formula (B) where R⁷ is independently hydrogen, halogen, —OH, NH₂, N(Me)₂, or unsubstituted C₁₋₃ alkyl. In one embodiment, R¹ is a moiety of formula (C) where R⁷ is independently hydrogen, halogen, NH₂, N(Me)₂, or unsubstituted C₁₋₃ alkyl. In one such embodiment, R⁷ is independently halogen or NH₂.

In one embodiment, R² is L¹-O-L²-R⁸, R^{8A}-substituted or unsubstituted C₁₋₃ alkyl, or R^{8B}-substituted or unsubstituted 4-10 membered heterocycle. In one embodiment, R² is hydrogen or L¹-O-L²-R⁸. In another embodiment, R² is R^{8A}-substituted or unsubstituted C₁₋₃ alkyl or R^{8B}-substituted or unsubstituted 4-10 membered heterocycle. In another embodiment, R² is R^{8B}-substituted or unsubstituted 4-6 membered heterocycle. In still another embodiment, R² is L¹-O-L²-R⁸, R^{8A}-substituted or unsubstituted C₁₋₃ alkyl, or R^{8B}-substituted or unsubstituted 4-6 membered heterocycle comprising one nitrogen heteroatom.

In one embodiment, R² is hydrogen where when R¹ is R⁷-substituted indazolyl, and n and m are 1, then p is not zero and R⁶ is not hydrogen.

In one embodiment, the compound of formula I has formula:

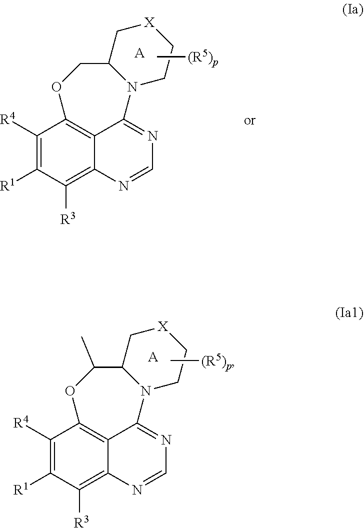

or a stereoisomer, atropisomer, tautomer, or pharmaceutically acceptable salt thereof, where R¹, R³, R⁴, R⁵, X, and p are as described herein. In one embodiment, the compound of formula (Ia1) has formula:

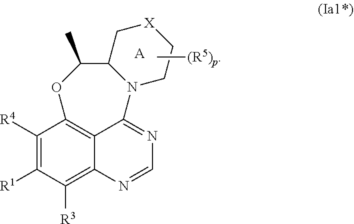

In another embodiment, the compound of formula (I) has formula:

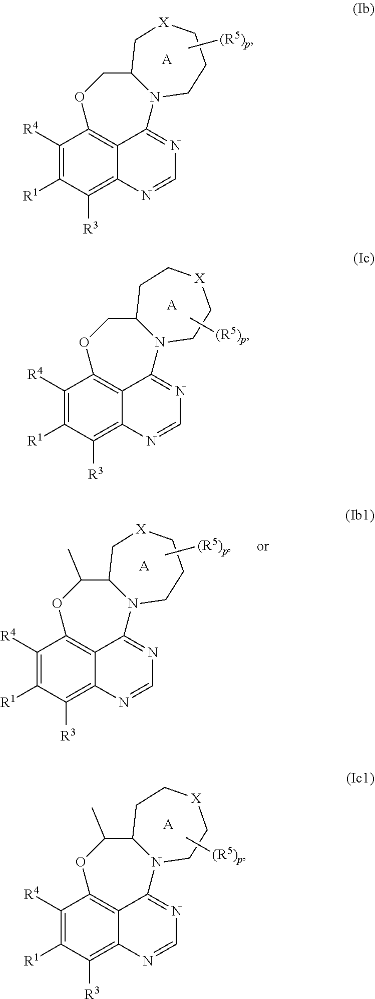

or a stereoisomer, atropisomer, tautomer, or pharmaceutically acceptable salt thereof, where R¹, R³, R⁴, R⁵, X, and p are as described herein. In one embodiment, the compound of formula (Ib1) and formula (Ic1), respectively, have formula:

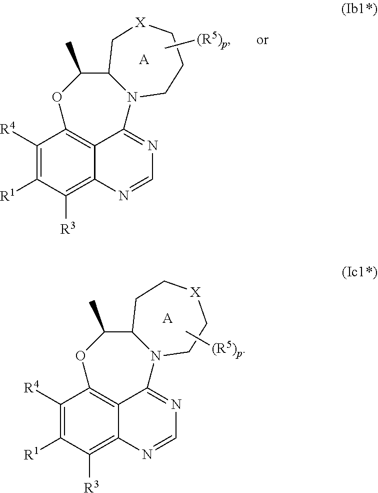

In one embodiment, R² is L¹-O-L²-R⁸. In one embodiment, one of L¹ and L² is a bond. In one embodiment, L¹ is a bond. In another embodiment, L² is a bond. In one embodiment, L¹ is unsubstituted C₁₋₃ alkylene and L² is a bond. In another embodiment, L¹ and L² are each independently C₁₋₃ alkylene.

In one embodiment where R² is L¹-O-L²-R⁸, L¹ is a bond and L² is unsubstituted C₁. 3 alkylene. In one such embodiment, L² is methylene. In one such embodiment, R⁸ is R⁹-substituted C₁₋₃ alkyl. In another such embodiment, R⁸ is R⁹-substituted or unsubstituted 4-10 membered heterocycle comprising N, S, or O.

In one embodiment, the compound of formula (I) has formula:

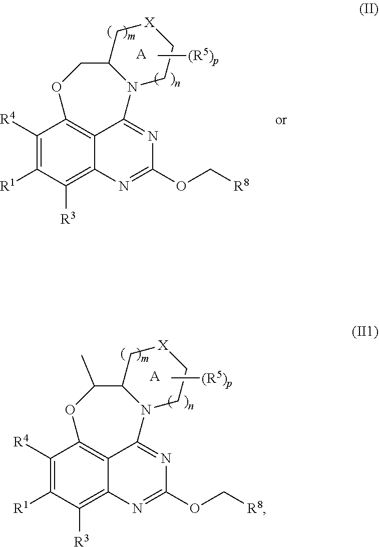

or a stereoisomer, atropisomer, tautomer, or pharmaceutically acceptable salt thereof, where R¹, R³, R⁴, R⁵, R⁸, X, n, m, and p are as described herein. In one embodiment, the compound for formula (II1) has formula:

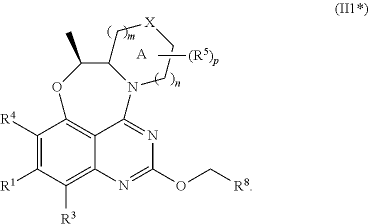

In one such embodiment, the compound of formula (II) has formula:

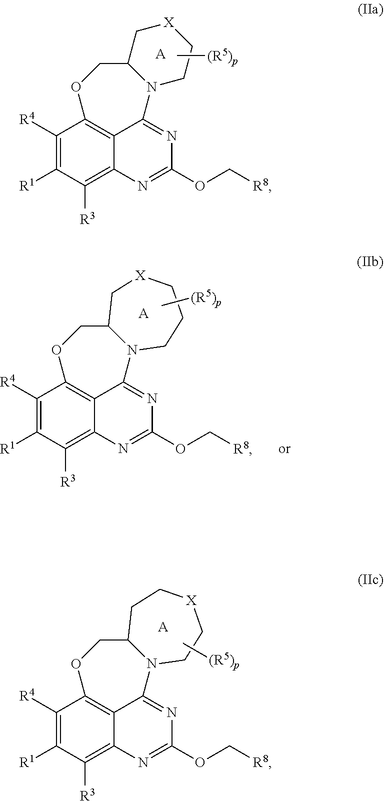

or a stereoisomer, atropisomer, tautomer, or pharmaceutically acceptable salt thereof, where R¹, R³, R⁴, R⁵, R⁸, X, and p are as described herein.

In one such embodiment, the compound of formula (II1) has formula:

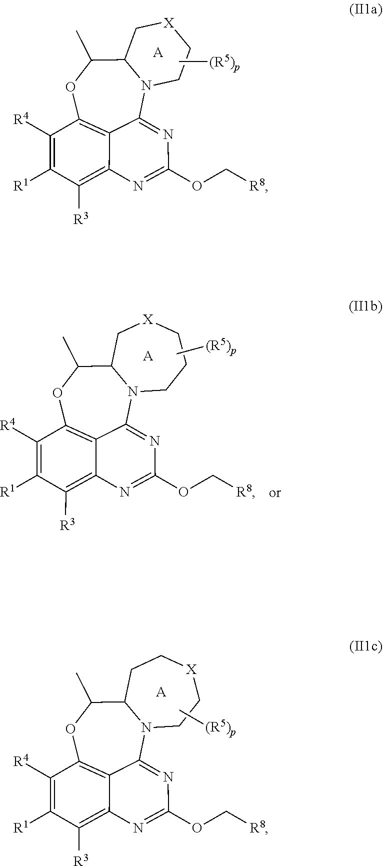

or a stereoisomer, atropisomer, tautomer, or pharmaceutically acceptable salt thereof, where R¹, R³, R⁴, R⁵, R⁸, X, and p are as described herein.

In one such embodiment, the compound of formula (II1) has formula:

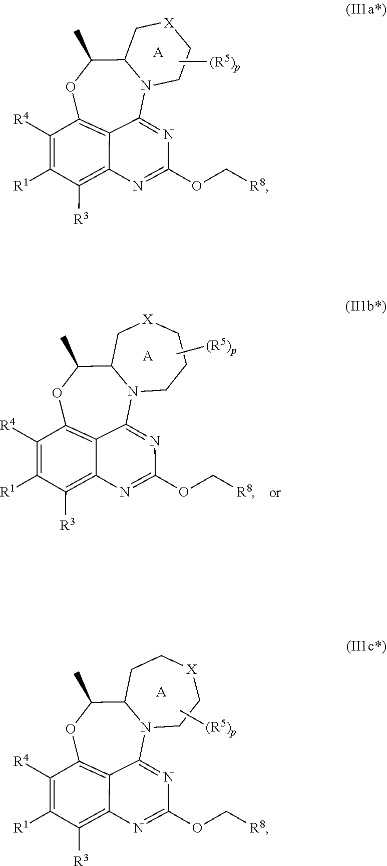

or a stereoisomer, atropisomer, tautomer, or pharmaceutically acceptable salt thereof, where R¹, R³, R⁴, R⁵, R⁸, X, and p are as described herein.

In one embodiment where R² is L¹-O-L²-R⁸, R⁸ is R⁹-substituted 4-10 membered heterocycle comprising N, S, or O. In another such embodiment, R⁸ is 4-10 membered heterocycle comprising one N heteroatom. In another such embodiment, R⁸ is 4, 5, 6, or 7 membered monocyclic heterocycle comprising one N heteroatom. In another such embodiment, R⁸ is 5 or 6 membered monocyclic heterocycle comprising one N heteroatom. In another such embodiment, R⁸ is 5 or 6 membered monocyclic heterocycle comprising one O heteroatom. In another such embodiment, R⁸ is a 6, 7, 8, or 9 membered fused bicyclic heterocycle comprising one N heteroatom. In another such embodiment, R⁸ is 7 or 8 membered fused bicyclic heterocycle comprising one N heteroatom. In another such embodiment, R⁸ is 7 or 8 membered fused bicyclic heterocycle comprising one N heteroatom and one O heteroatom. In one embodiment, R⁸ is pyrrolidinyl or tetrahydrofuranyl.

In such embodiments, each R⁹ is independently halogen, oxo, unsubstituted C₁₋₃ alkyl, unsubstituted C₁₋₃ haloalkyl, unsubstituted C₁₋₃ alkoxy, or R¹⁰-substituted or unsubstituted C₁₋₃ alkylidene. In another such embodiment, each R⁹ is independently halogen, oxo, or R¹⁰-substituted or unsubstituted C₁₋₃ alkylidene. In one embodiment, each R⁹ is independently unsubstituted C₁₋₃ alkyl or unsubstituted C₁₋₃ alkoxy. In one embodiment, each R⁹ is R¹⁰-substituted or unsubstituted C₃₋₄ cycloalkyl or R¹⁰-substituted or unsubstituted 3 or 4-membered heterocycle. In one embodiment, two R⁹ together form an R¹⁰-substituted or unsubstituted C₃₋₅ cycloalkyl. In one such embodiment, two R⁹ together form a R¹⁰-substituted cyclopropyl. In one such embodiment, two R⁹ together form a R¹⁰-substituted cyclopropyl where R¹⁰ is halogen (e.g. F or Cl). In one embodiment, where two R⁹ together form a R¹⁰-substituted cyclopropyl, the cyclopropyl is attached at a single carbon of R⁸. In one embodiment, two R⁹ together form a R¹⁰-substituted cyclopropyl, the cyclopropyl is attached at two separate carbon atoms of R⁸. In another such embodiment, two R⁹ together form a unsubstituted C₃₋₅ heterocycle comprising one or more oxygen atoms. In one such embodiment, the heterocycle is a 1,3-dioxolanyl.

In one embodiment, R¹⁰ is hydrogen or halogen. In one embodiment, R¹⁰ is hydrogen. In another embodiment, R¹⁰ is halogen. In one such embodiment, R¹⁰ is F.

In one embodiment, where R² is L¹-O-L²-R⁸, R⁸ is

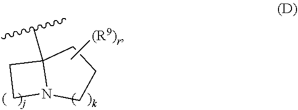

or a stereoisomer thereof, wherein,
R⁹ is halogen, —OCF₃, —OCHF₂, —OCH₂F, R¹⁰-substituted or unsubstituted C₁₋₃ alkylidene, or two R⁹ together form a R¹⁰-substituted or unsubstituted C₃₋₅ cycloalkyl;
r is an integer of 0-12;
j is 1, 2, or 3; and
k is 1 or 2.

In one embodiment, where R² is L¹-O-L²-R⁸, R⁸ is

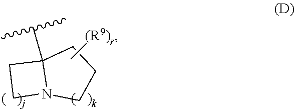

or a stereoisomer thereof, wherein,
R⁹ is halogen or R¹⁰-substituted or unsubstituted C₁₋₃ alkylidene;
r is an integer of 0-12;
j is 1, 2, or 3; and
k is 1 or 2.

In one such embodiment, r is 0, 1, 2, 3, or 4. In another such embodiment, r is 0, 1, 2, or 3. In one embodiment, R⁸ is

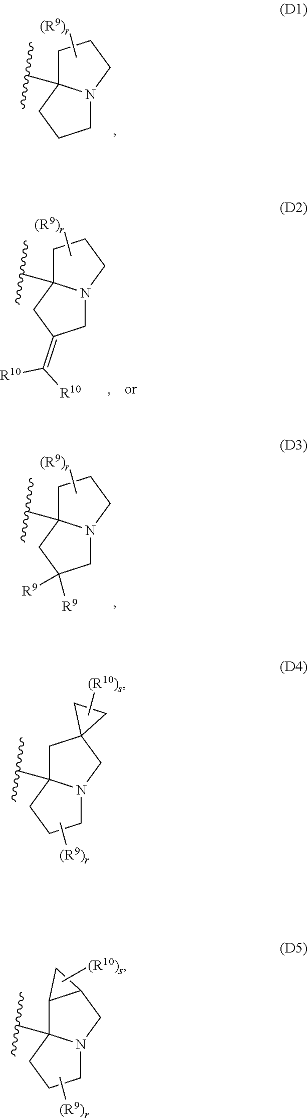

or a stereoisomer thereof, where R⁹, R¹⁰ and r are as described herein and s is 1 or 2. In one such embodiment, r is 0, 1, 2, 3, or 4. In another such embodiment, r is 0, 1, 2, or 3. In one embodiment, R⁸ has formula D1, D2, or D3, where R⁹, R¹⁰ and r are as described herein.

In one such embodiment, R⁹ is independently halogen or R¹⁰-substituted or unsubstituted C₁₋₃ alkylidene; each R¹⁰ is independently hydrogen or halogen; and r is 1 or 2.

In one embodiment, R⁸ is

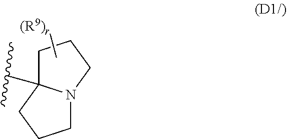

or a stereoisomer thereof, where r is 0.

In another embodiment, R⁸ is

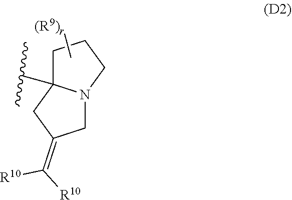

or a stereoisomer thereof, where r is 0 and each R¹⁰ is independently hydrogen or F. In one such embodiment, r is 0 and each R¹⁰ is hydrogen. In another such embodiment, r is 0 and each R¹⁰ is F. In another such embodiment, r is 0 where one R¹⁰ is hydrogen and one R¹⁰ is F. In another such embodiment, each R¹⁰ is independently hydrogen or F, r is 1 or 2, and R⁹ is F.

In another embodiment, R⁸ is

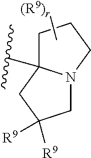

or a stereoisomer thereof, where r is 0 and each R⁹ is independently hydrogen or halogen. In one such embodiment, each R⁹ is F and r is 0. In one such embodiment, each R⁹ is F and r is 1.

In another embodiment where R² is L¹-O-L²-R⁸, R⁸ is

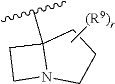

or a stereoisomer thereof. In one such embodiment, r is 1 and R⁹ is halogen, oxo, or unsubstituted C₁ alkylidene. In one such embodiment, two R⁹ together form a R¹⁰-substituted or unsubstituted C₃₋₅ cycloalkyl.

In one embodiment, R⁸ is

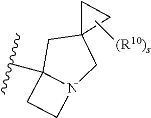

or a stereoisomer thereof, where R¹⁰ is halogen and s is 1 or 2. In one such embodiment, R⁸ is

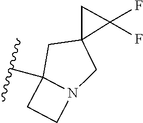 [SMILES: *C12CCN1CC1(C2)CC1(F)F]

or a stereoisomer thereof.

In another embodiment where R² is L¹-O-L²-R⁸, R⁸ is

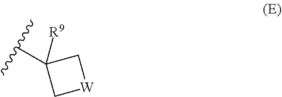

or a stereoisomer thereof, wherein
R⁹ is hydrogen or unsubstituted C₁₋₃ alkyl; and
W is O, SO₂, or NR¹²; and
R¹² is hydrogen, unsubstituted C₁₋₃ alkyl, or unsubstituted C₁₋₃ haloalkyl.

In one such embodiment, W is O and R⁹ is methyl. In another such embodiment, W is NR¹², where R¹² is unsubstituted C₁₋₃ haloalkyl and R⁹ is hydrogen. In another such embodiment, W is SO₂ and R⁹ is hydrogen.

In one embodiment of the compounds or a pharmaceutically acceptable salt thereof described herein, R⁸ is azetidinyl, oxetanyl, or thietanedioxide.

In further embodiments provided herein, R⁸ is a moiety having formula:

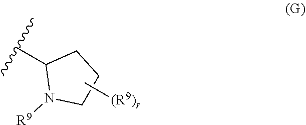

or a stereoisomer thereof, wherein,
   R⁹ is independently halogen, oxo, or unsubstituted C₁₋₃ alkyl; and
   r is 1 or 2.

In one such embodiment, R⁸ is a moiety having formula (G) where R⁹ and r are as described herein. In one such embodiment, R⁹ is oxo and r is 1. In another such embodiment, R⁹ is F and r is 1 or 2.

In another embodiment, R⁸ is a moiety having formula:

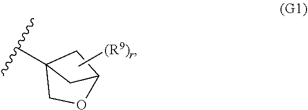

or a stereoisomer thereof, wherein R⁹ and r as described herein.

In another embodiment, R⁸ is a moiety having formula:

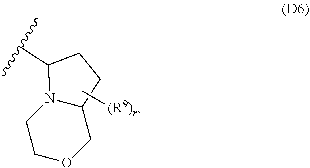

or a stereoisomer thereof, wherein R⁹ and r are as described herein.

In another embodiment, R⁸ is a moiety having formula:

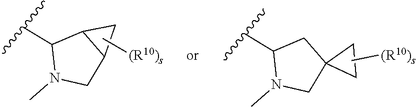

or a stereoisomer thereof, where R¹⁰ is halogen and s is 1 or 2.

In still another embodiment, R⁸ is R⁹-substituted or unsubstituted C₁₋₃ alkyl. In one such embodiment, R⁸ is a moiety of formula:

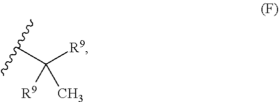

or a stereoisomer thereof, where each R⁹ is independently unsubstituted C₁₋₃ alkyl or unsubstituted C₁₋₃ alkoxy.

In another embodiment, R⁸ is a moiety having formula:

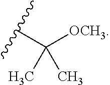 [SMILES: *C(C)(C)OC]

In one embodiment, R⁸ is:

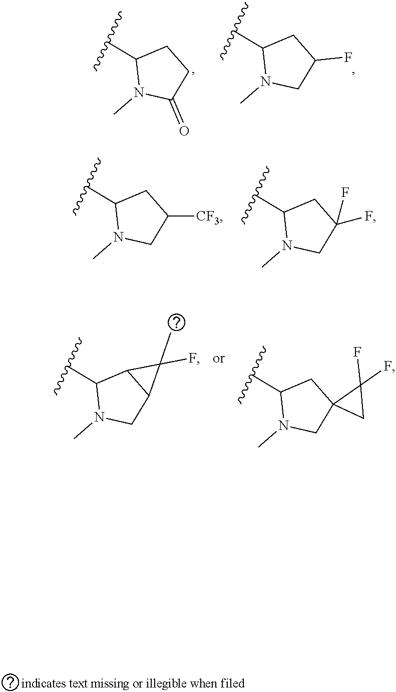

or a stereoisomer thereof.

In one embodiment, R⁸ is:

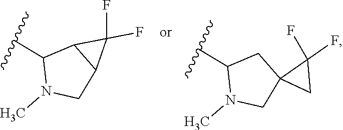

or a stereoisomer thereof.

In one embodiment, R⁸ is:

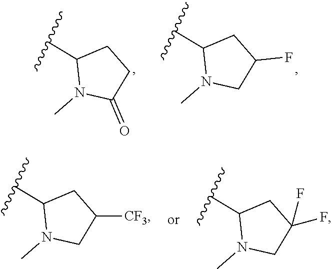

or a stereoisomer thereof.

In one embodiment, R⁸ is:

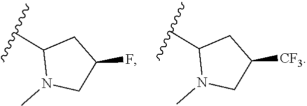

In another embodiment, R⁸ is:

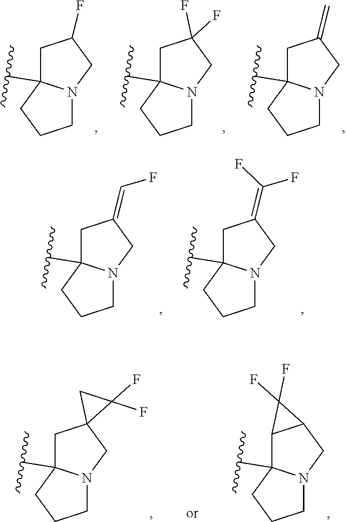

or a stereoisomer thereof.

In another embodiment, R⁸ is:

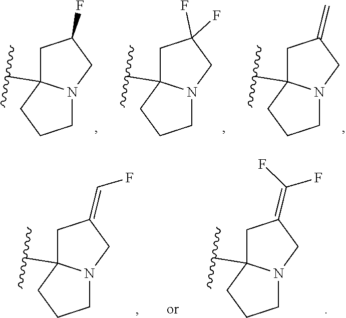

In another embodiment, R⁸ is:

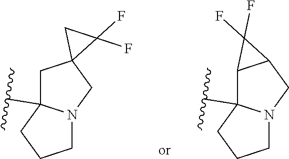 [SMILES: *C12CCCN1CC1(C2)CC1(F)F ; *C12CCCN1CC1C2C1(F)F]

or a stereoisomer thereof.

In another embodiment, R⁸ is:

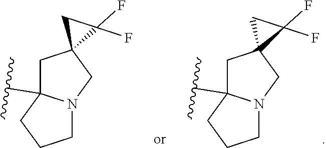

In still another embodiment, R⁸ is:

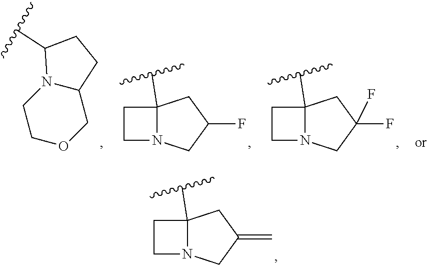

or a stereoisomer thereof.

In still another embodiment, R⁸ is:

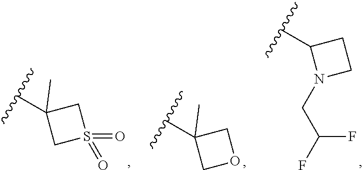

or a stereoisomer thereof.

In still another embodiment, R⁸ is:

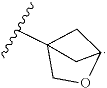 [SMILES: *C12COC(C1)C2]

In still another embodiment, R⁸ is:

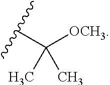 [SMILES: *C(C)(C)OC]

In still another embodiment, R² is:

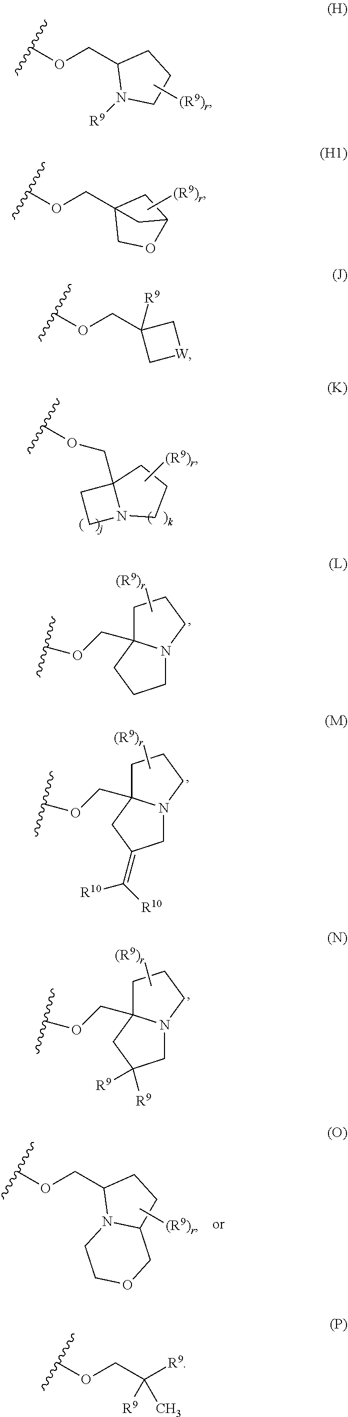

or a stereoisomer thereof, where R⁹, R¹⁰, r, j, and k are as described herein. In one embodiment, R⁹ is halogen or R¹⁰-substituted or unsubstituted C₁₋₃ alkylidene. In another such embodiment, R⁹ is halogen, oxo, R¹⁰-substituted or unsubstituted C₁₋₃ alkylidene, and r is independently 0, 1, or 2.

In one embodiment, R² is:

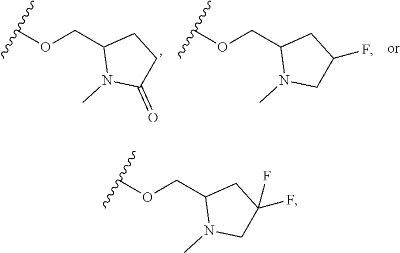

or a stereoisomer thereof.

In another embodiment, R² is:

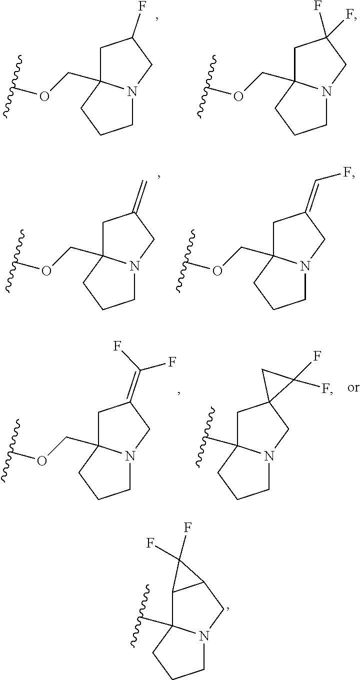

or a stereoisomer thereof.

In another embodiment, R² is:

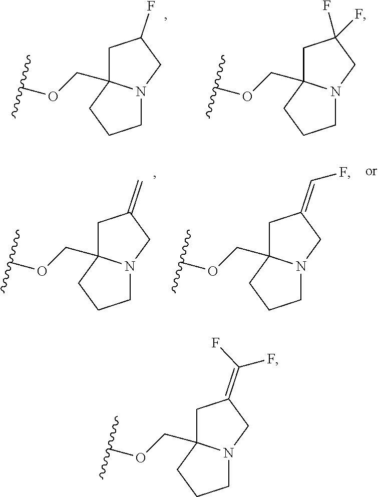

or a stereoisomer thereof.

In still another embodiment, R² is:

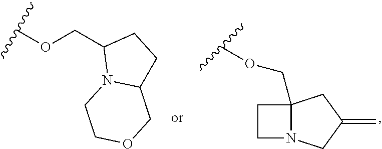 [SMILES: *OCC1CCC2COCCN12 ; *OCC12CCN1CC(=C)C2]

or a stereoisomer thereof.

In still another embodiment, R² is:

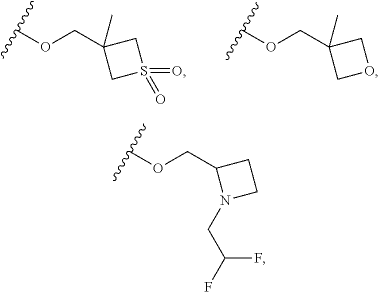

or a stereoisomer thereof.

In still another embodiment, R² is:

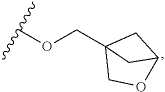 [SMILES: *OCC12COC(C1)C2]

In still another embodiment, R² is:

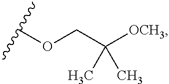 [SMILES: *OCC(C)(C)OC]

In another embodiment, R² is R^{8A}-substituted or unsubstituted C₁₋₃ alkyl or R^{8B}-substituted or unsubstituted 4-10 membered heterocycle. In one embodiment, each R^{8A} is independently R^{9A}-substituted or unsubstituted C₁₋₃ alkyl or R^{9A}-substituted or unsubstituted C₁₋₃ alkoxy. In one embodiment, each R^{8A} is independently R^{8A} is independently R^{9A}-substituted or unsubstituted alkoxy or R^{9A}-substituted or unsubstituted 4-6 membered heterocycle In another embodiment, each R^{8A} is independently R^{9A}-substituted or unsubstituted C₃₋₄ cycloalkyl, or R^{9A}-substituted or unsubstituted 4-6 membered heterocycle. In one embodiment, R^{9A} is R⁹-substituted or unsubstituted 4-10 membered heterocycle comprising N. In another embodiment, R⁹ is independently halogen, unsubstituted C₁₋₃ alkyl, or R¹⁰-substituted or unsubstituted C₁₋₃ alkylidene.

In one embodiment, R² is R^{8A}-substituted or unsubstituted C₁₋₃ alkyl, where R^{8A} is R^{9A}-substituted or unsubstituted C₁₋₃ alkoxy, R^{9A}-substituted or unsubstituted C₃₋₄ cycloalkyl, or R^{9A}-substituted or unsubstituted 4-6 membered heterocycle.

In one embodiment, R^{9A} is independently halogen, oxo, unsubstituted C₁₋₃ alkyl, unsubstituted C₁₋₃ haloalkyl, unsubstituted C₁₋₃ alkoxy, or unsubstituted C₁₋₃ alkylidene. In another such embodiment, R^{9A} is independently R^{9A} is independently halogen, oxo, or unsubstituted C₁₋₃ alkylidene. In still another embodiment, R^{9A} is R⁹-substituted or unsubstituted 4-10 membered heterocycle comprising N, S, or O.

In one embodiment, R² is R^{8A}-substituted or unsubstituted C₁₋₃ alkyl, where R^{8A} is R^{9A}-substituted or unsubstituted C₁₋₃ alkyl.

In one embodiment, R² is R^{8A}-substituted or unsubstituted C₁₋₃ alkyl, where R^{8A} is R^{9A}-substituted or unsubstituted C₁₋₃ alkoxy. In one such embodiment, R^{9A} is independently R⁹-substituted or unsubstituted C₃₋₄ cycloalkyl, or R⁹-substituted or unsubstituted 4-10 membered heterocycle comprising one N heterocycle. In another such embodiment, R^{9A} is independently R⁹-substituted or unsubstituted 5 or 6 membered monocyclic heterocycle comprising one N heterocycle or 7 or 8 membered fused bicyclic heterocycle comprising one N heterocycle. In such embodiments, R⁹ is independently halogen, oxo, unsubstituted C₁₋₃ alkyl, or R¹⁰-substituted or unsubstituted C₁₋₃ alkylidene, where R¹⁰ is as described herein.

In another embodiment, R² is R^{8A}-substituted or unsubstituted C₁₋₃ alkyl, where R^{8A} is R^{9A}-substituted or unsubstituted C₃₋₄ cycloalkyl. In one embodiment, each R^{8B} is independently halogen, oxo, unsubstituted C₁₋₃ alkyl, unsubstituted C₁₋₃ haloalkyl, unsubstituted C₁₋₃ alkoxy, or unsubstituted C₁₋₃ alkylidene.

In one embodiment, R² is R^{8B}-substituted or unsubstituted 4-10 membered heterocycle. In one such embodiment, R^{8B} is halogen, oxo, or unsubstituted C₁₋₃ alkylidene. In one embodiment, R² is R^{8B}-substituted or unsubstituted 4, 5, or 7 membered heterocycle comprising one N heteroatom.

In one such embodiment, R² is:

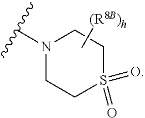

In one such embodiment, R² is:

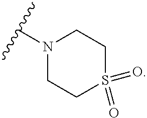 [SMILES: *N1CCS(=O)(=O)CC1]

In one embodiment, R³ and R⁴ are each independently hydrogen, —CN, halogen, unsubstituted C₁₋₃ alkyl, or unsubstituted cyclopropyl. In one embodiment, R³ and R⁴ are each independently hydrogen, halogen, or unsubstituted C₁₋₃ alkyl. In one embodiment, R³ and R⁴ are each independently hydrogen or halogen. In one embodiment, both R³ and R⁴ are not hydrogen. In another embodiment, one of R³ and R⁴ is hydrogen and the other is halogen. In one embodiment, R³ is halogen. In one such embodiment, R³ is F or Cl. In another embodiment, R⁴ is hydrogen. In another embodiment, R⁴ is halogen. In one such embodiment, R⁴ is F or Cl.

In one embodiment, each R⁵ is independently halogen, oxo, unsubstituted C₁₋₃ alkyl, or unsubstituted C₁₋₃ haloalkyl. In one embodiment, p is 1 and R⁵ is halogen, oxo, or unsubstituted C₁₋₃ alkyl. In another embodiment, R⁵ is independently oxo or unsubstituted C₁₋₃ alkyl, and p is 1. In one such embodiment, n and m together make a 6 or 7 membered ring where p is 1. In another such embodiment, n and m together make a 7 membered ring where p is 0. In one embodiment, n and m together make a 6 membered ring. In one such embodiment, n and m together make a 6 membered ring where p is 0 or 1. In still another embodiment, n and m together make a 7 membered ring. In one such embodiment, n and m together make a 7 membered ring where p is 0 or 1.

In one embodiment, p is 0.

In one embodiment, two R⁵ together form a bridge between two carbon atoms of ring A, wherein the bridge comprises 1-3 carbons. In one embodiment, two R⁵ together form a bridge between two carbon atoms of ring A, wherein the bridge comprises 1 or 2 carbons. In one embodiment, the bridge comprises 1 carbon. In another embodiment, the bridge comprises 2 carbons. In one such embodiment, the compound of formula (I) has formula:

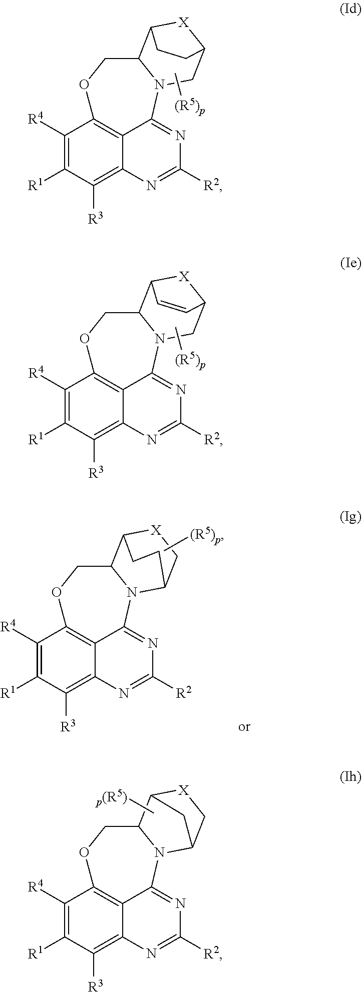

or a stereoisomer, atropisomer, tautomer, or pharmaceutically acceptable salt thereof, where R¹, R², R³, R⁴, R⁵, X, and p are as described herein. In one such embodiment, p is 0.

In another such embodiment, the compound of formula (I) has formula:

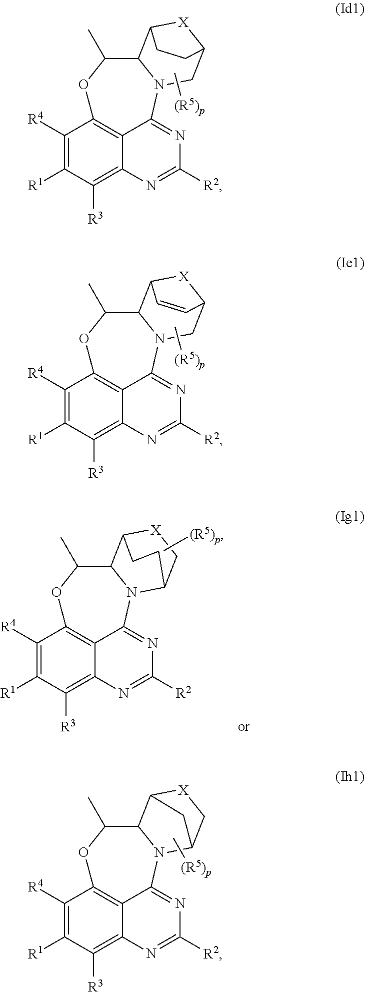

or a stereoisomer, atropisomer, tautomer, or pharmaceutically acceptable salt thereof, where R¹, R², R³, R⁴, R⁵, X, and p are as described herein. In one such embodiment, p is 0.

In another such embodiment, the compound of formula (I) has formula:

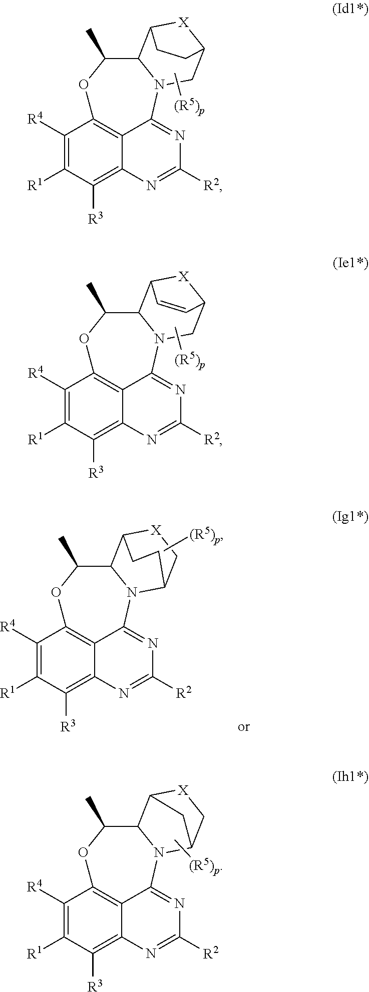

or a stereoisomer, atropisomer, tautomer, or pharmaceutically acceptable salt thereof, where R¹, R², R³, R⁴, R⁵, X, and p are as described herein. In one such embodiment, p is 0.

In such embodiments, R¹ is as described herein. In another such embodiment, R¹ is a moiety of formula (A1), (A2), or (B). In another embodiment, R² is a moiety of formula (H), (J), (K), (L), (M), (N), (O), or (P). In such embodiments, X is NR⁶, where R⁶ is hydrogen, methyl, or a moiety of formula (Q), (R), or (S).

In another such embodiment, the compound is a compound of formula (II) having formula;

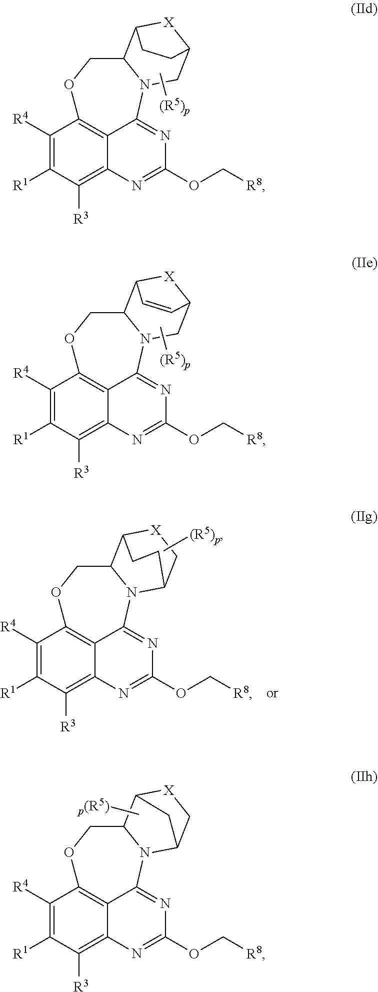

or a stereoisomer, atropisomer, tautomer, or pharmaceutically acceptable salt thereof, where R¹, R³, R⁴, R⁵, R⁸, X, and p are as described herein. In one such embodiment, p is 0.

In another such embodiment, the compound is a compound of formula (II1) having formula

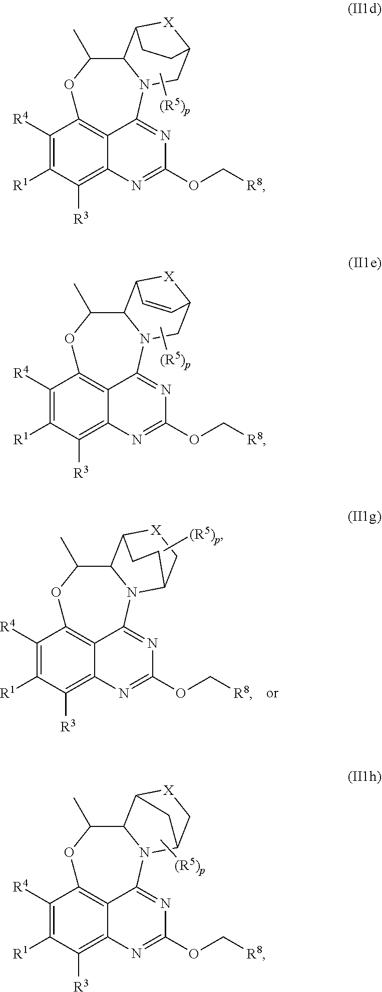

or a stereoisomer, atropisomer, tautomer, or pharmaceutically acceptable salt thereof, where R¹, R³, R⁴, R⁵, R⁸, X, and p are as described herein. In one such embodiment, p is 0.

In another such embodiment, the compound is a compound of formula (II1) having formula;

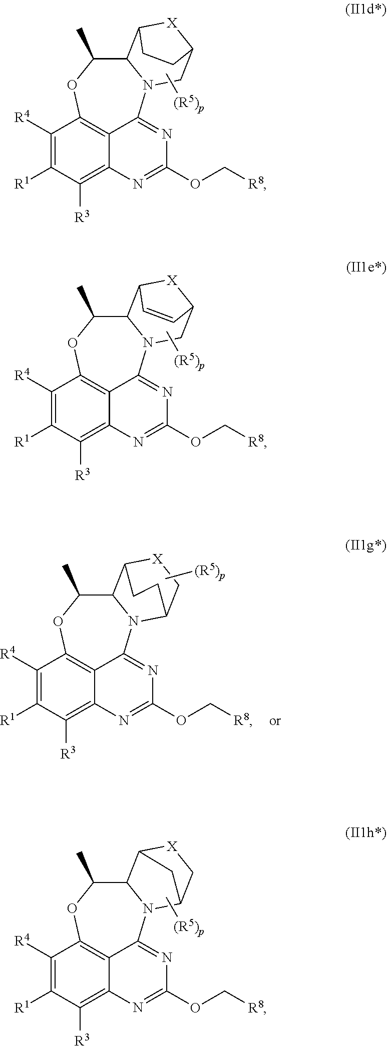

or a stereoisomer, atropisomer, tautomer, or pharmaceutically acceptable salt thereof, where R¹, R³, R⁴, R⁵, R⁸, X, and p are as described herein. In one such embodiment, p is 0.

In one such embodiment, R¹ is as described herein. In another such embodiment, R¹ is a moiety of formula (A1), (A2), (B), or (C). In another such embodiment, R⁸ is a moiety of formula (D1), (D2), (D3), (D4), (D5), (E), (G), or (G1). In one embodiment, R⁸ is a moiety of formula (F). In such embodiments, X is NR⁶, where R⁶ is hydrogen, methyl, or a moiety of formula (Q), (R), or (S).

In still another embodiment, two R⁵ together form a bridge between two carbon atoms of ring A, wherein the bridge comprises one of O or NR¹¹. In one embodiment, the bridge comprises O. In another such embodiment, the bridge comprises NR¹¹, where R¹¹ is hydrogen, C(O)CH₃, or methyl.

In one embodiment of the compounds of formula (I) or a stereoisomer, atropisomer, tautomer, or pharmaceutically acceptable salt thereof, X is NR⁶. In one embodiment, R⁶ is hydrogen. In one embodiment, R⁶ is R^{6A}-substituted or unsubstituted C₁₋₆ alkyl. In one embodiment, R⁶ is hydrogen or R^{6A}-substituted or unsubstituted C₁₋₆ alkyl, R^{6A}-substituted or unsubstituted C₁₋₆ alkenyl; or R^{6A}-substituted or unsubstituted C₁₋₆ alkynyl. In one embodiment, R⁶ is hydrogen or R^{6A}-substituted or unsubstituted C₁₋₆ alkyl, unsubstituted C₁₋₆ alkenyl; or unsubstituted C₁₋₆ alkynyl. In another embodiment, R⁶ is hydrogen or R^{6A}-substituted or unsubstituted C₁₋₆ alkyl. In still another embodiment, R⁶ is hydrogen or unsubstituted C₁₋₆ alkyl.

In one such embodiment, where R⁶ is R^{6A}-substituted or unsubstituted C₁₋₆ alkyl, R^{6A} is halogen, CN, OR^{6B}, S(O)₂R^{6C}, unsubstituted C₁₋₃ alkyl; or R^{6B}-substituted or unsubstituted 4 membered heterocycle. In another such embodiment, where R⁶ is R^{6A}-substituted or unsubstituted C₁₋₆ alkyl, R^{6A} is halogen, CN, OR^{6B}, unsubstituted C₁₋₃ alkyl; or R^{6B}-substituted or unsubstituted 4 membered heterocycle. In another such embodiment, where R⁶ is R^{6A}-substituted or unsubstituted C₁₋₆ alkyl, R^{6A} is halogen, CN, OH, OMe, OEt, OCF₃, SO₂Me, unsubstituted C₁₋₃ alkyl, or 4-membered heterocycle.

In another such embodiment, where R⁶ is R^{6A}-substituted or unsubstituted C₁₋₆ alkyl, R^{6A} is F, Cl, CN, CH₃, OH, OCH₃, OCF₃, SCH₃, SO₂CH₃, or a combination thereof. In one embodiment, where R⁶ is R^{6A}-substituted or unsubstituted C₁₋₆ alkyl, R^{6A} is halogen or oxetanyl. In another embodiment, R⁶ is unsubstituted C₁₋₆ alkyl (e.g. methyl).

In another embodiment, R⁶ is R^{6A}-substituted or unsubstituted 3-4 membered heterocycle. In one such embodiment, R⁶ is azetidinyl or oxetanyl. In one embodiment, R⁶ is unsubstituted oxetanyl.

In another embodiment, R⁶ is R^{6A}-substituted or unsubstituted C₁₋₃ haloalkyl. In another embodiment, R⁶ is R^{6A}-substituted or unsubstituted C₁₋₃ alkenyl. In another embodiment, R⁶ is R^{6A}-substituted or unsubstituted C₁₋₃ alkynyl. In one embodiment, R⁶ is hydrogen. In another embodiment, R⁶ is methyl.

In one embodiment of the compounds or a stereoisomer, atropisomer, tautomer, or pharmaceutically acceptable salt thereof described herein R⁶ is hydrogen, methyl, or a moiety of formula:

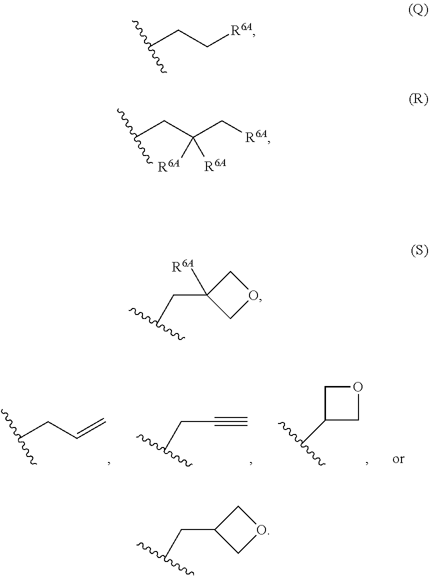

In one such embodiment, R^{6A} is F, Cl, CN, CH₃, OH, OCH₃, OCF₃, SCH₃, SO₂CH₃.

In another embodiment, R⁶ is

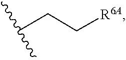

where R^{6A}, is CH₂F, CN, OH, OCH₃, OCF₃, SCH₃, SO₂CH₃.

In another embodiment, R is

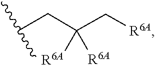

where where R^{6A}, is independently F, CH₃, or OCH₃.

In another embodiment, R⁶ is

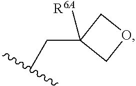

where R^{6A} is hydrogen, CH₃ or F.

In still another embodiment, R⁶ is hydrogen, methyl, or a moiety of formula:

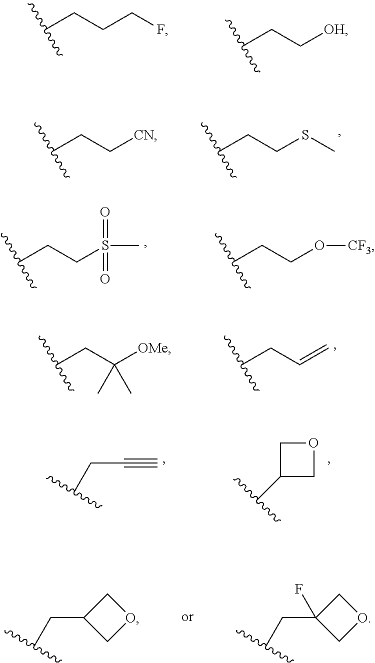

In one embodiment, R^{6B} and R^{6C} are each independently C₁₋₃ alkyl.

In one embodiment, the compound of formula (I) has formula:

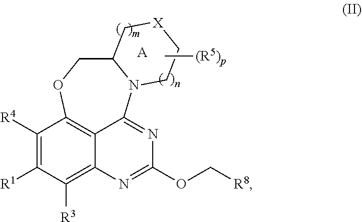

or a stereoisomer, atropisomer, tautomer, or pharmaceutically acceptable salt thereof, wherein R¹ is a moiety of formula (A1), (A2), or (B); R⁸ is a moiety of formula (D1), (D2), (D3), (E), (G), or (G1); and X is NR⁶, where R⁶ is hydrogen, methyl, or a moiety of formula (Q), (R), or (S). In another embodiment, p is 0. In another embodiment, p is 1 and R⁵ is oxo or unsubstituted C₁₋₃ alkyl. In still another embodiment, R⁴ is C₁₋₆ alkyl. In still another embodiment, m and n are each 1. In still another embodiment, m is 2 and n is 1. In still another embodiment, m is 1 and n is 2. In still another embodiment, two R⁵ together make a 1-2 carbon bridge as described herein.

In one embodiment, the compound of formula (I) has formula:

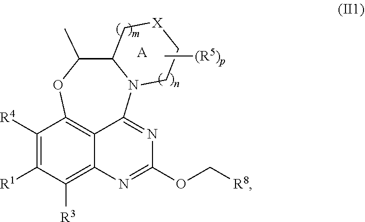

or a stereoisomer, atropisomer, tautomer, or pharmaceutically acceptable salt thereof, wherein R¹ is a moiety of formula (A1), (A2), or (B); R⁸ is a moiety of formula (D1), (D2), (D3), (E), (G), or (G1); and X is NR⁶, where R⁶ is hydrogen, methyl, or a moiety of formula (Q), (R), or (S). In another embodiment, p is 0. In another embodiment, p is 1 and R⁵ is oxo or unsubstituted C₁₋₃ alkyl. In still another embodiment, R⁴ is C₁₋₆ alkyl. In still another embodiment, m and n are each 1. In still another embodiment, m is 2 and n is 1. In still another embodiment, m is 1 and n is 2. In still another embodiment, two R⁵ together make a 1-2 carbon bridge as described herein. In one embodiment, the compound of formula (II1) has formula (II1*) as described herein.

In one embodiment, the compound has formula:

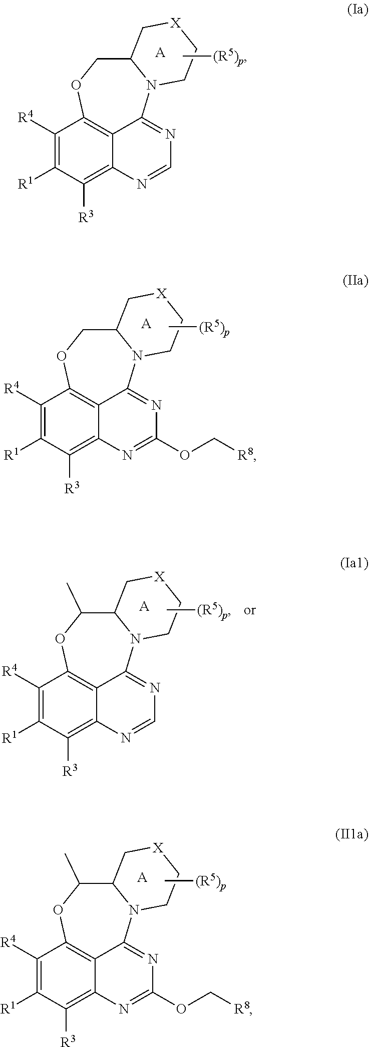

or a stereoisomer, atropisomer, tautomer, or pharmaceutically acceptable salt thereof, wherein R¹ is a moiety of formula (A1), (A2), or (B); R⁸ is a moiety of formula (D1), (D2), (D3), (D4), (D5), (E), (G), or (G1); and X is NR⁶, where R⁶ is hydrogen, methyl, or a moiety of formula (Q), (R), or (S). In another embodiment, p is 0. In another embodiment, p is 1 and R⁵ is oxo or unsubstituted C₁₋₃ alkyl. In still another embodiment, R⁴ is C₁₋₆ alkyl. In still another embodiment, two R⁵ together make a 1-2 carbon bridge as described herein.

In one embodiment, the compound has formula:

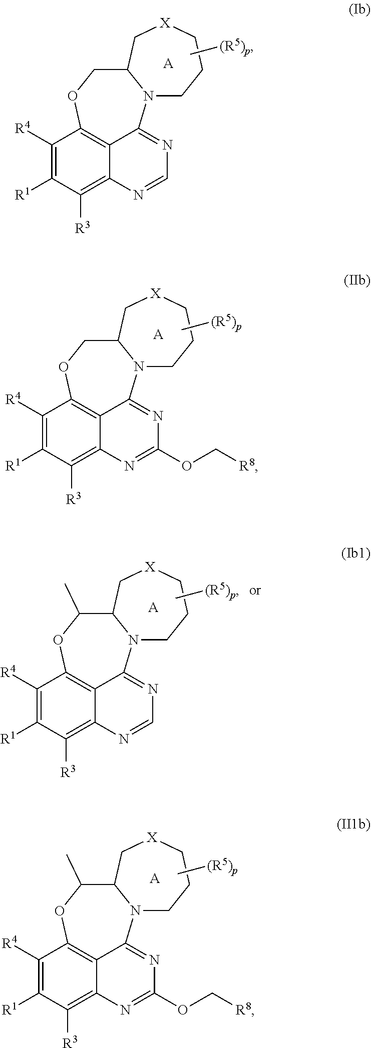

or a stereoisomer, atropisomer, tautomer, or pharmaceutically acceptable salt thereof, wherein R¹ is a moiety of formula (A1), (A2), or (B); R⁸ is a moiety of formula (D1), (D2), (D3), (D4), (D5), (E), (G), or (G1); and X is NR⁶, where R⁶ is hydrogen, methyl, or a moiety of formula (Q), (R), or (S). In another embodiment, p is 0. In another embodiment, p is 1 and R⁵ is oxo or unsubstituted C₁₋₃ alkyl. In still another embodiment, R⁴ is C₁₋₆ alkyl.

In one embodiment, the compound has formula:

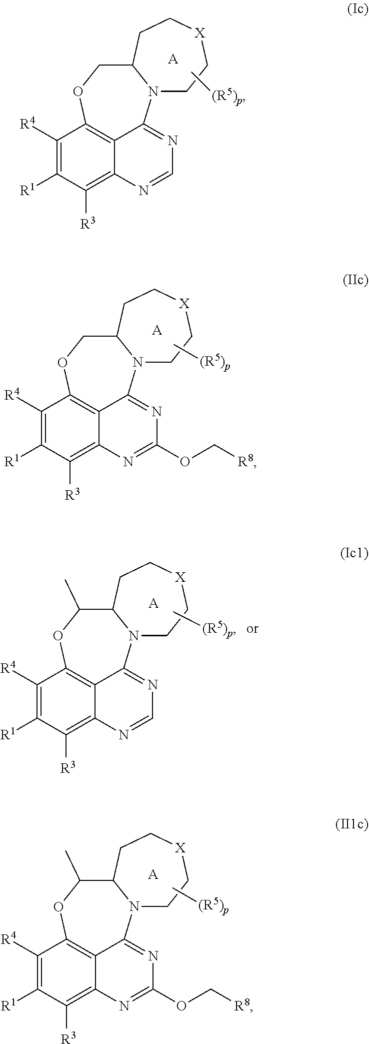

or a stereoisomer, atropisomer, tautomer, or pharmaceutically acceptable salt thereof, wherein R¹ is a moiety of formula (A1), (A2), or (B); R⁸ is a moiety of formula (D1), (D2), (D3), (D4), (D5), (E), (G), or (G1); and X is NR⁶, where R⁶ is hydrogen, methyl, or a moiety of formula (Q), (R), or (S). In another embodiment, p is 0. In another embodiment, p is 1 and R⁵ is oxo or unsubstituted C₁₋₃ alkyl. In still another embodiment, R⁴ is C₁₋₆ alkyl.

In one embodiment, the compound has formula:

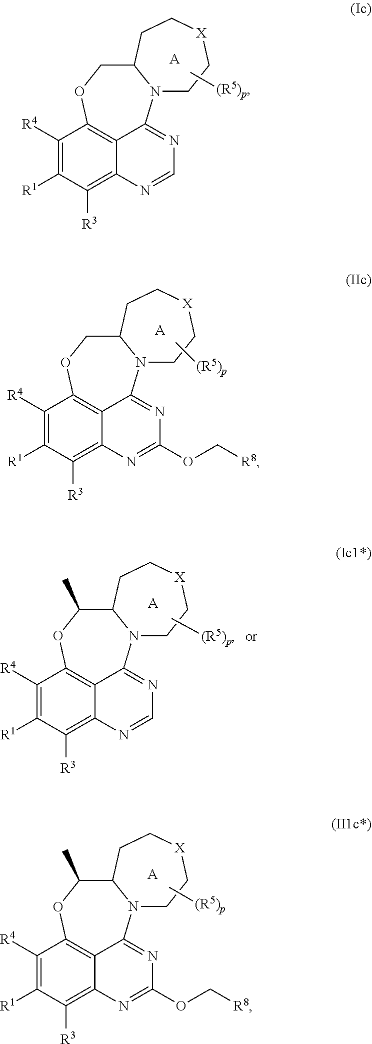

or a stereoisomer, atropisomer, tautomer, or pharmaceutically acceptable salt thereof, wherein R¹ is a moiety of formula (A1), (A2), or (B); R⁸ is a moiety of formula (D1), (D2), (D3), (D4), (D5), (E), (G), or (G1); and X is NR⁶, where R⁶ is hydrogen, methyl, or a moiety of formula (Q), (R), or (S). In another embodiment, p is 0. In another embodiment, p is 1 and R⁵ is oxo or unsubstituted C₁₋₃ alkyl. In still another embodiment, R⁴ is C₁₋₆ alkyl.

In one embodiment, the compound has formula:

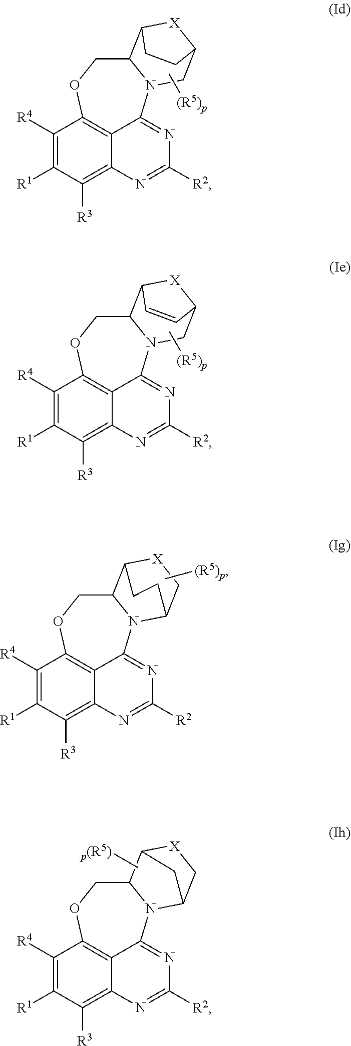

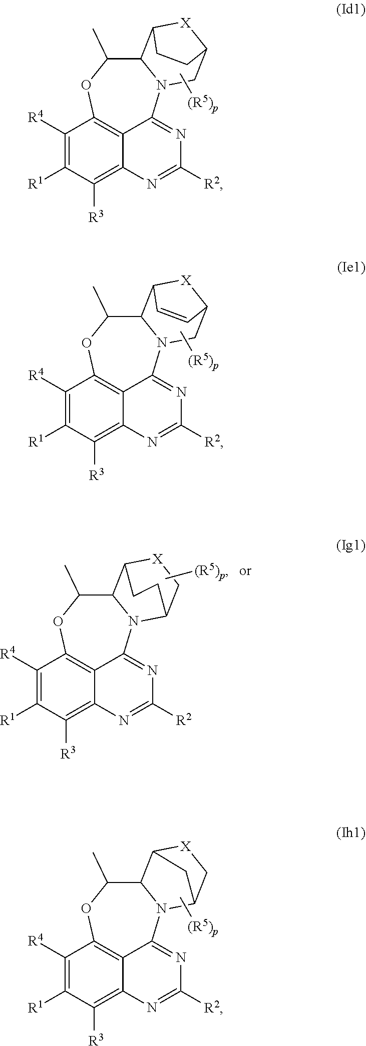

or a stereoisomer, atropisomer, tautomer, or pharmaceutically acceptable salt thereof, wherein R¹ is a moiety of formula (A1), (A2), (B), or (C); R² is a moiety of formula (H), (J), (K), (L), (M), or (N) and X is NR⁶, where R⁶ is a moiety of formula (Q), (R), or (S). In another embodiment, p is 0. In another embodiment, p is 1 and R⁵ is oxo or unsubstituted C₁₋₃ alkyl. In still another embodiment, R⁴ is C₁₋₆ alkyl.

In one embodiment, the compound has formula:

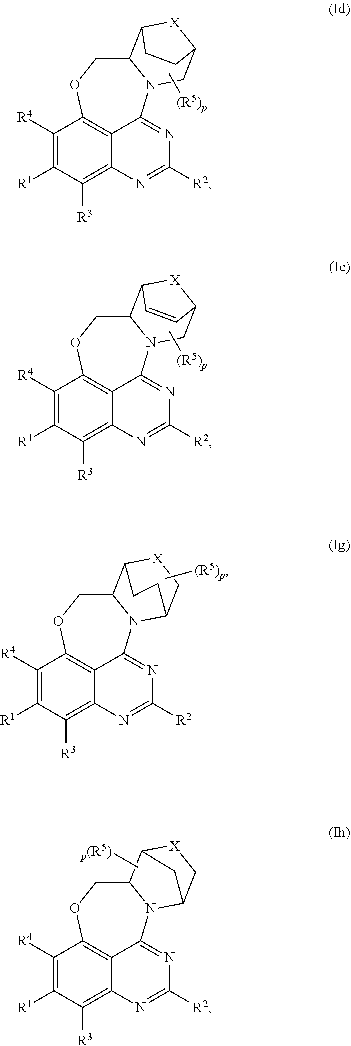

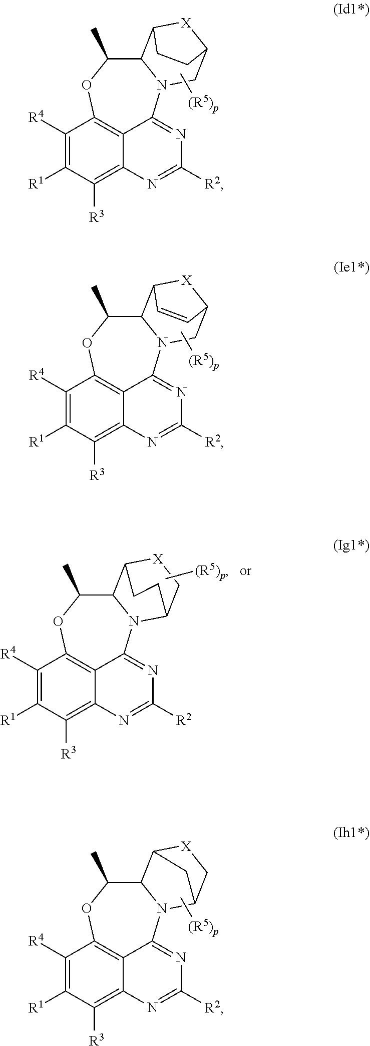

or a stereoisomer, atropisomer, tautomer, or pharmaceutically acceptable salt thereof, wherein R¹ is a moiety of formula (A1), (A2), (B), or (C); R² is a moiety of formula (H), (J), (K), (L), (M), or (N) and X is NR⁶, where R⁶ is a moiety of formula (Q), (R), or (S). In another embodiment, p is 0. In another embodiment, p is 1 and R⁵ is oxo or unsubstituted C₁₋₃ alkyl. In still another embodiment, R⁴ is C₁₋₆ alkyl.

In one embodiment of the compounds or a pharmaceutically acceptable salt thereof of formula (Id), (Ie), (Ig), (Ih), (Id1), (Ie1), (Ig1), (Ih1), (Id1*), (Ie1*), (Ig1*), and (Ih1*), R¹ is a moiety of formula (A1) where each R^{7A} is independently hydrogen, halogen, methyl, or CF₃, as described herein. In one embodiment of the compounds or a pharmaceutically acceptable salt thereof of formula (Id), (Ie), (Ig), and (Ih), R¹ is a moiety of formula (B) where each R⁷ is halogen or methyl as described herein. In such embodiments, R² is a moiety of formula (L), (M), or (N). In another such embodiment, R² is a moiety of formula (H1), (J), (K), or (O). In another such embodiment, R² is a moiety of formula (P). In such embodiments, X is NR⁶, where R⁶ is hydrogen, methyl, or a moiety of formula (Q), (R), or (S). In one embodiment, the compounds or a pharmaceutically acceptable salt thereof of formula (Id), (Ie), (Ig), (Ih), (Id1), (Ie1), (Ig1), (Ih1), (Id1*), (Ie1*), (Ig1*), and (Ih1*) or a stereoisomer, atropisomer, tautomer, or pharmaceutically acceptable salt thereof, R¹ is a moiety of formula (A1), (A2), (B), or (C); R⁸ is a moiety of formula (D), (D1), (D2), (D3), (E), or (G); and X is NR⁶, where R⁶ is a moiety of formula (Q), (R), or (S). In another embodiment, p is 0. In another embodiment, p is 1 and R⁵ is oxo or unsubstituted C₁₋₃ alkyl. In still another embodiment, R⁴ is C₁₋₆ alkyl.

In one embodiment of the compounds or a pharmaceutically acceptable salt thereof of formula (Id), (Ie), (Ig), (Ih), (Id1), (Ie1), (Ig1), (Ih), (Id1*), (Ie1*), (Ig1*), and (Ih1*), R¹ is a moiety of formula (A1) where each R^{7A} is independently hydrogen, halogen, methyl, or CF₃, as described herein. In one embodiment of the compounds or a pharmaceutically acceptable salt thereof of formula (IId), (IIe), (IIg), and (IIh), R¹ is a moiety of formula (B) where each R⁷ is halogen or methyl as described herein. In such embodiments, R⁸ is a moiety of formula (D1), (D2), (D3), (D4), (D5), (E), (G), or (G1). In another such embodiment, R⁸ is a moiety of formula (F). In such embodiments, X is NR⁶, where R⁶ is hydrogen, methyl, or a moiety of formula (Q), (R), or (S).

In one embodiment, R⁸ of the compounds described herein is a moiety of formula (D), (D6), (G), or (E) as described herein.

In one embodiment, R⁸ of the compounds described herein is a moiety of formula D1, D2, or D3 as described herein.

In another embodiment, R⁸ of the compounds described herein is a moiety of formula D6 as described herein.

In one embodiment, R⁸ of the compounds described herein is:

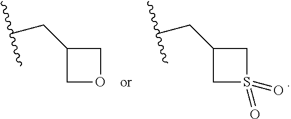 [SMILES: *CC1COC1 ; *CC1CS(=O)(=O)C1]

Further provided herein are compounds of formula:

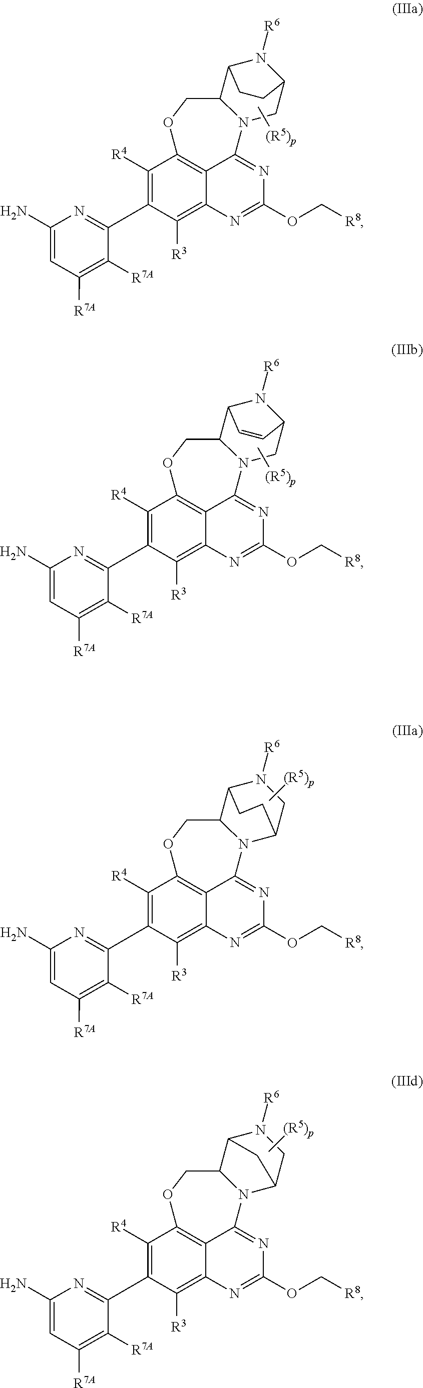

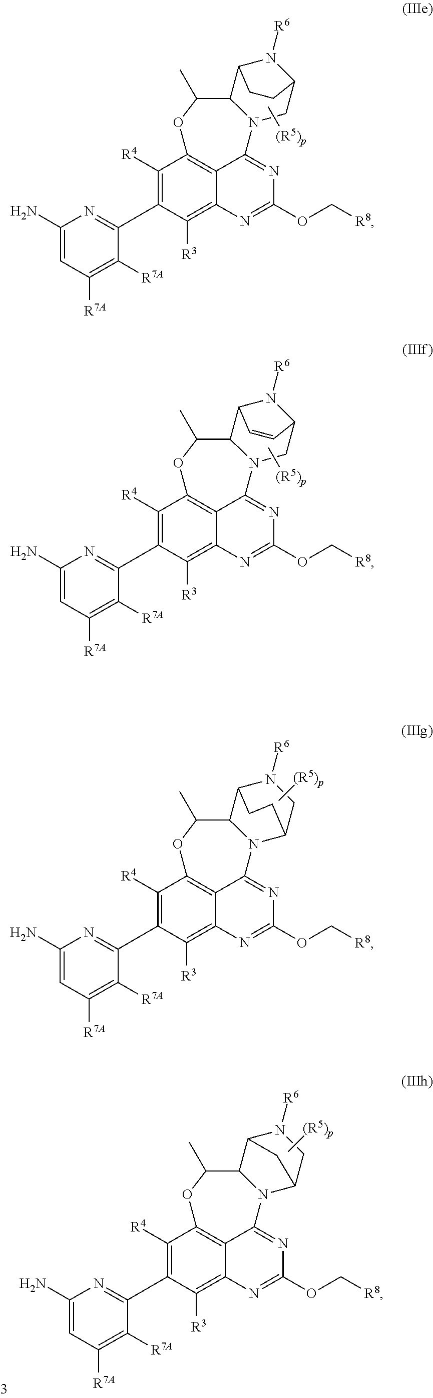

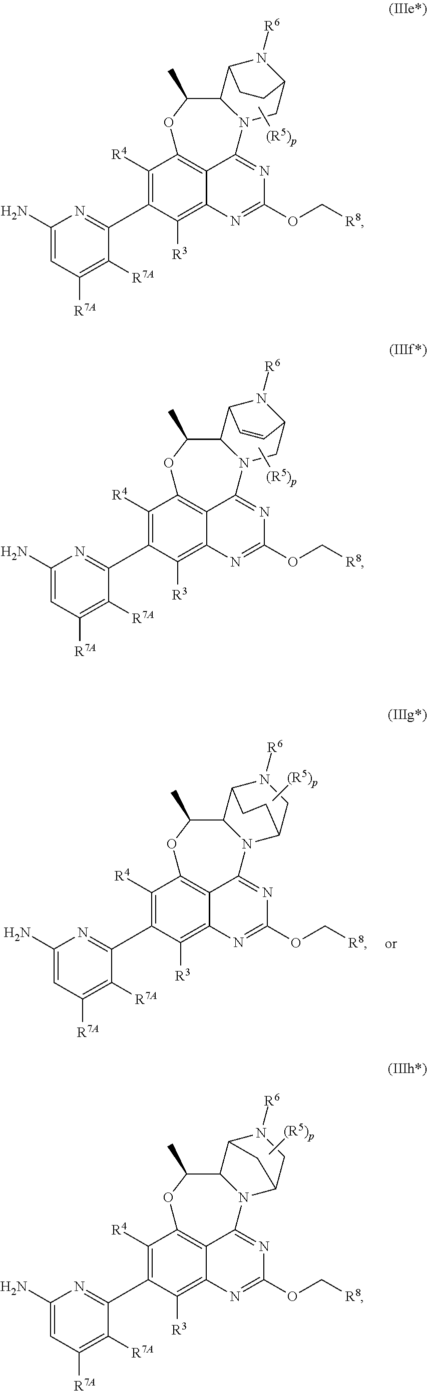

wherein R³, R⁴, R⁵, R^{7A}, X, and P are as defined herein and R⁸ is:

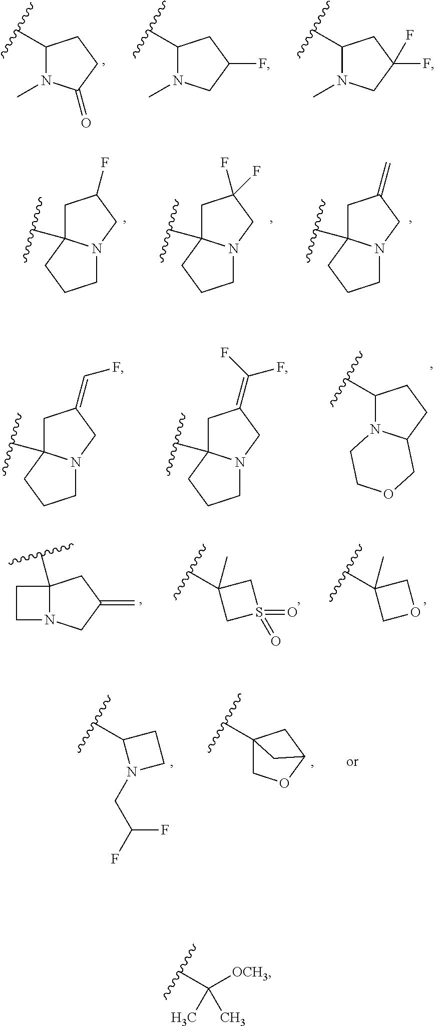

or a stereoisomer thereof.

In one embodiment, a compound of formula (I) or a stereoisomer, atropisomer, tautomer, or pharmaceutically acceptable salt thereof as described herein is at least 5, 10, 25, 50, 100, 250, 500, 700, 1000, 1300, 1500, 2000, or 3000× selective for the KRas G12D mutated protein over wildtype (WT) KRas protein.

In one embodiment, the compound of formula (I) or a stereoisomer, atropisomer, tautomer, or pharmaceutically acceptable salt thereof is a compound of Table 1.

TABLE 1

| Cmpd | | | MS |
|---|---|---|---|
| No | Structure | Chemical Name | (M + H) |
| 1A | 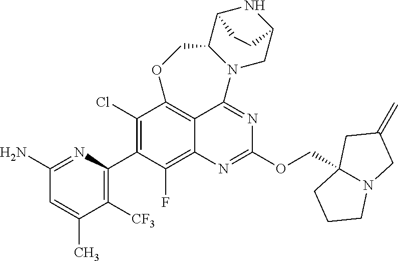 | 6-((2R,5aS,6S,9R)-3-chloro-1- fluoro-13-(((S)-2- methylenetetrahydro-1H- pyrrolizin-7a(5H)-yl)methoxy)- 5a,6,7,8,9,10-hexahydro-5H-6,9- epiminoazepino[2′,1′:3,4][1,4] oxazepino[5,6,7-de]quinazolin- 2-yl)-4-methyl-5- (trifluoromethyl)pyridin-2-amine | 646 |
| 1B | 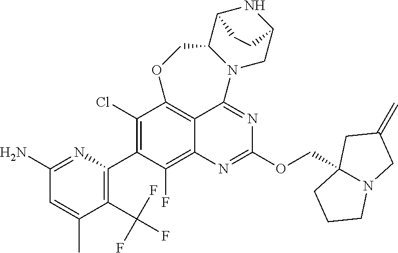 | 6-((2S,5aS,6S,9R)-3-chloro-1- fluoro-13-(((S)-2- methylenetetrahydro-1H- pyrrolizin-7a(5H)-yl)methoxy)- 5a,6,7,8,9,10-hexahydro-5H-6,9- epiminoazepino[2′, 1′:3,4][1,4] oxazepino[5,6,7-de]quinazolin- 2-yl)-4-methyl-5- (trifluoromethyl)pyridin-2-amine | 646 |
| 2 | 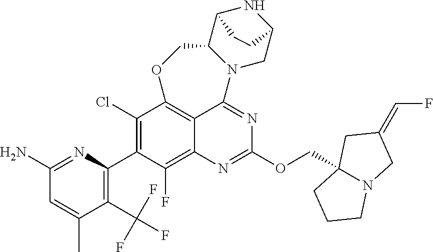 | 6-((2R,5aS,6S,9R)-3-chloro-1- fluoro-13-(((S,Z)-2- (fluoromethylene)tetrahydro-1H- pyrrolizin-7a(5H)-yl)methoxy)- 5a,6,7,8,9,10-hexahydro-5H-6,9- epiminoazepino[2′, 1′:3,4][1,4] oxazepino[5,6,7-de]quinazolin- 2-yl)-4-methyl-5- (trifluoromethyl)pyridin-2-amine | 664 |
| 3 | 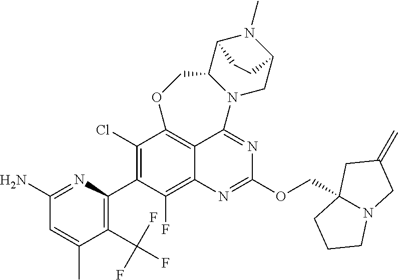 | 6-((2R,5aS,6S,9R)-3-chloro-1- fluoro-15-methyl-13-(((S)-2- methylenetetrahydro-1H- pyrrolizin-7a(5H)-yl)methoxy)- 5a,6,7,8,9,10-hexahydro-5H-6,9- epiminoazepino[2′, 1′:3,4][1,4] oxazepino[5,6,7-de]quinazolin- 2-yl)-4-methyl-5- (trifluoromethyl)pyridin-2-amine | 660 |
| 4 | 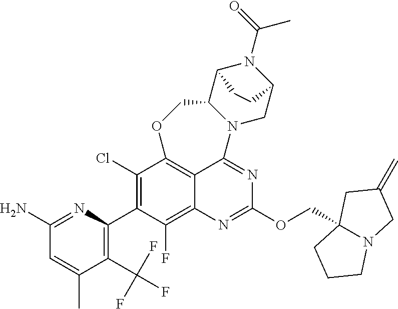 | 1-((2R,5aS,6S,9R)-2-(6-amino-4- methyl-3-(trifluoromethyl)pyridin- 2-yl)-3-chloro-1-fluoro-13-(((S)-2- methylenetetrahydro-1H- pyrrolizin-7a(5H)-yl)methoxy)- 5a,6,7,8,9,10-hexahydro-5H-6,9- epiminoazepino[2′, 1′:3,4][1,4] oxazepino[5,6,7-de]quinazolin-15- yl)ethan-1-one | 688 |
| 5 | 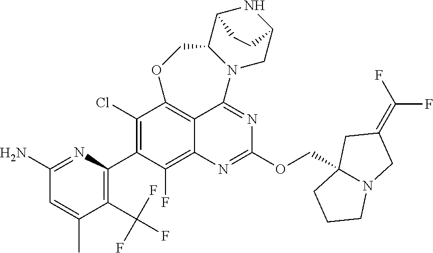 | 6-((2R,5aS,6S,9R)-3-chloro-13- (((S)-2- (difluoromethylene)tetrahydro- 1H-pyrrolizin-7a(5H)- yl)methoxy)-1-fluoro- 5a,6,7,8,9,10-hexahydro-5H-6,9- epiminoazepino[2′,1′:3,4][1,4] oxazepino[5,6,7-de]quinazolin- 2-yl)-4-methyl-5- (trifluoromethyl)pyridin-2-amine | 682 |
| 6 | 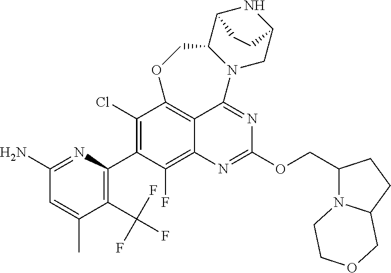 | 6-((2R,5aS,6S,9R)-3-chloro-1- fluoro-13-((hexahydro-1H- pyrrolo[2,1-c][1,4]oxazin-6- yl)methoxy)-5a,6,7,8,9,10- hexahydro-5H-6,9- epiminoazepino[2′,1′:3,4][1,4] oxazepino[5,6,7-de]quinazolin- 2-yl)-4-methyl-5- (trifluoromethyl)pyridin-2-amine | 650 |
| 6A | 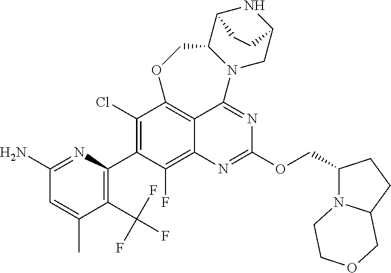 | | |
| 6B | 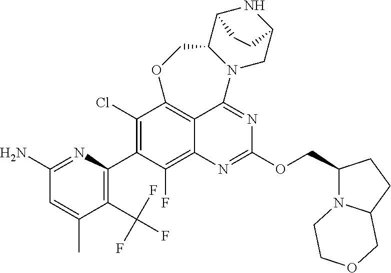 | | |
| 7 | 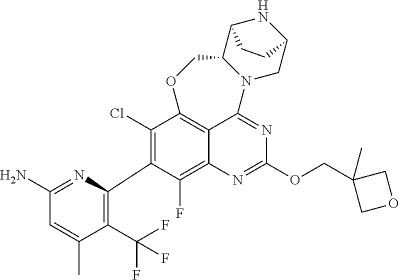 | 6-((2R,5aS,6S,9R)-3-chloro-1- fluoro-13-((3-methyloxetan-3- yl)methoxy)-5a,6,7,8,9,10- hexahydro-5H-6,9- epiminoazepino[2′,1′:3,4][1,4] oxazepino[5,6,7-de]quinazolin- 2-yl)-4-methyl-5- (trifluoromethyl)pyridin-2-amine | 595 |
| 8 | 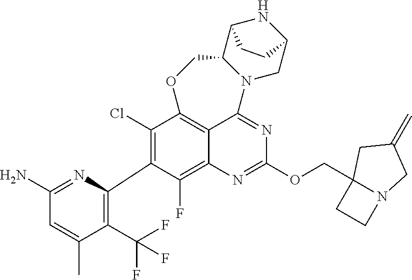 | 6-((2R,5aS,6S,9R)-3-chloro-1- fluoro-13-((3-methylene-1- azabicyclo[3.2.0]heptan-5- yl)methoxy)-5a,6,7,8,9,10- hexahydro-5H-6,9- epiminoazepino[2′,1′:3,4][1,4] oxazepino[5,6,7-de]quinazolin- 2-yl)-4-methyl-5- (trifluoromethyl)pyridin-2-amine | 632 |
| 8A | 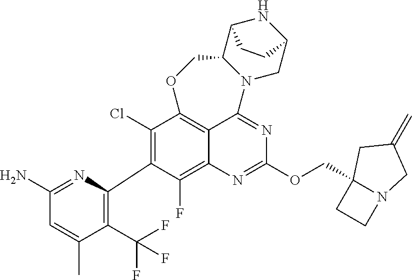 | | |
| 8B | 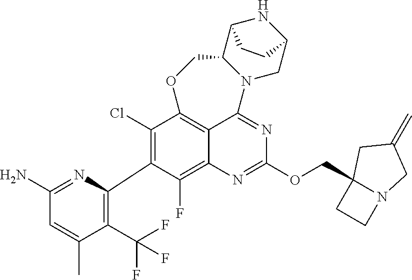 | | |
| 9 | 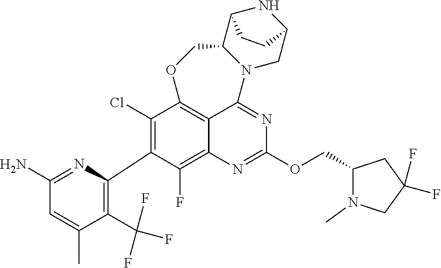 | 6-((2R,5aS,6S,9R)-3-chloro-13- (((S)-4,4-difluoro-1- methylpyrrolidin-2-yl)methoxy)-1- fluoro-5a,6,7,8,9,10-hexahydro- 5H-6,9- epiminoazepino[2′,1′:3,4][1,4] oxazepino[5,6,7-de]quinazolin- 2-yl)-4-methyl-5- (trifluoromethyl)pyridin-2-amine | 644 |
| 10 | 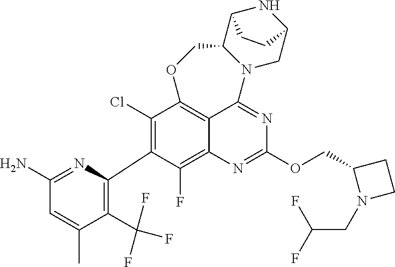 | 6-((2R,5aS,6S,9R)-3-chloro-13- (((S)-1-(2,2- difluoroethyl)azetidin-2- yl)methoxy)-1-fluoro- 5a,6,7,8,9,10-hexahydro-5H-6,9- epiminoazepino[2′,1′:3,4][1,4] oxazepino[5,6,7-de]quinazolin- 2-yl)-4-methyl-5- (trifluoromethyl)pyridin-2-amine | 644 |
| 11 | 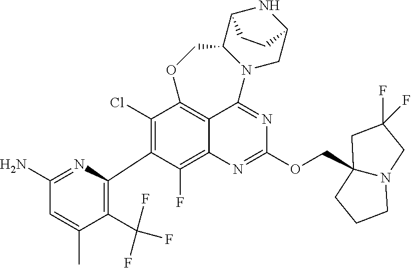 | 6-((2R,5aS,6S,9R)-3-chloro-13- (((R)-2,2-difluorotetrahydro-1H- pyrrolizin-7a(5H)-yl)methoxy)-1- fluoro-5a,6,7,8,9,10-hexahydro- 5H-6,9- epiminoazepino[2′, 1′:3,4][1,4] oxazepino[5,6,7-de]quinazolin- 2-yl)-4-methyl-5- (trifluoromethyl)pyridin-2-amine | 670 |
| 12 | 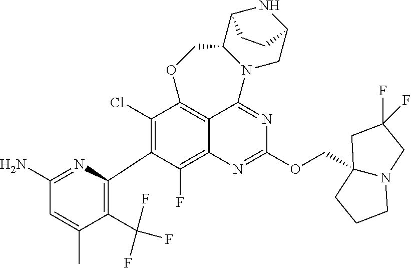 | 6-((2R,5aS,6S,9R)-3-chloro-13- (((S)-2,2-difluorotetrahydro-1H- pyrrolizin-7a(5H)-yl)methoxy)-1- fluoro-5a,6,7,8,9,10-hexahydro- 5H-6,9- epiminoazepino[2′,1′:3,4][1,4] oxazepino[5,6,7-de]quinazolin- 2-yl)-4-methyl-5- (trifluoromethyl)pyridin-2-amine | 670 |
| 13A | 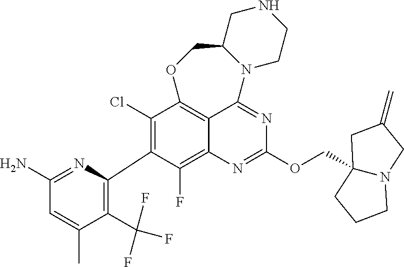 | 6-((10R,13aR)-11-chloro-9- fluoro-7-(((S)-2- methylenetetrahydro-1H- pyrrolizin-7a(5H)-yl)methoxy)- 1,2,3,4,13,13a- hexahydropyrazino[2′, 1′:3,4][1,4] oxazepino[5,6,7-de]quinazolin- 10-yl)-4-methyl-5- (trifluoromethyl)pyridin-2-amine | 620 |
| 13B | 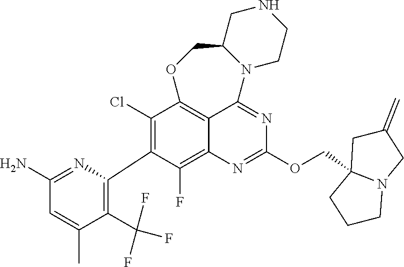 | 6-((10S,13aR)-11-chloro-9- fluoro-7-(((S)-2- methylenetetrahydro-1H- pyrrolizin-7a(5H)-yl)methoxy)- 1,2,3,4,13,13a- hexahydropyrazino[2′,1′:3,4][1,4] oxazepino[5,6,7-de]quinazolin- 10-yl)-4-methyl-5- (trifluoromethyl)pyridin-2-amine | 620 |
| 14 | 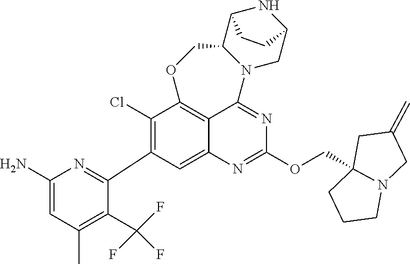 [SMILES: C=C1CN2CCC[C@@]2(COc2nc3c4c(c(Cl)c(-c5nc(N)cc(C)c5C(F)(F)F)cc4n2)OC[C@@H]2[C@@H]4CC[C@H](CN32)N4)C1] | 6-((5aS,6S,9R)-3-chloro-13- (((S)-2-methylenetetrahydro-1H- pyrrolizin-7a(5H)-yl)methoxy)- 5a,6,7,8,9,10-hexahydro-5H-6,9- epiminoazepino[2′,1′:3,4][1,4] oxazepino[5,6,7-de]quinazolin- 2-yl)-4-methyl-5- (trifluoromethyl)pyridin-2-amine | 628 |
| 15 | 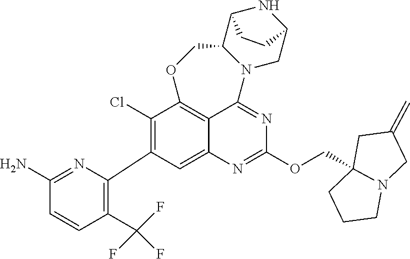 [SMILES: C=C1CN2CCC[C@@]2(COc2nc3c4c(c(Cl)c(-c5nc(N)ccc5C(F)(F)F)cc4n2)OC[C@@H]2[C@@H]4CC[C@H](CN32)N4)C1] | 6-((5aS,6S,9R)-3-chloro-13- (((S)-2-methylenetetrahydro-1H- pyrrolizin-7a(5H)-yl)methoxy)- 5a,6,7,8,9,10-hexahydro-5H-6,9- epiminoazepino[2′,1′:3,4][1,4] oxazepino[5,6,7-de]quinazolin-2-yl)- 5-(trifluoromethyl)pyridin-2-amine | 614 |
| 16A | 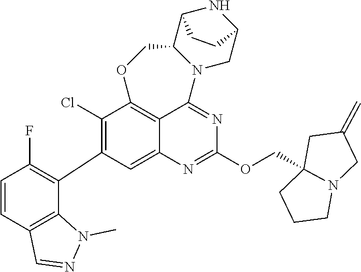 [SMILES: C=C1CN2CCC[C@@]2(COc2nc3c4c(c(Cl)c(-c5c(F)ccc6cnn(C)c56)cc4n2)OC[C@@H]2[C@@H]4CC[C@H](CN32)N4)C1] | (5aS,6S,9R)-3-chloro-2-(6-fluoro- 1-methyl-1H-indazol-7-yl)-13- (((S)-2-methylenetetrahydro-1H- pyrrolizin-7a(5H)-yl)methoxy)- 5a,6,7,8,9,10-hexahydro-5H-6,9- epiminoazepino[2′, 1′:3,4][1,4] oxazepino[5,6,7-de]quinazoline | 602 |
| 16B | 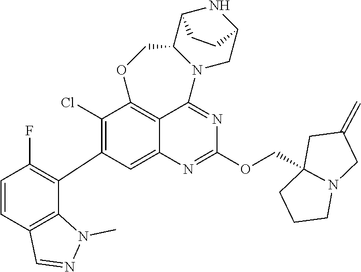 [SMILES: C=C1CN2CCC[C@@]2(COc2nc3c4c(c(Cl)c(-c5c(F)ccc6cnn(C)c56)cc4n2)OC[C@@H]2[C@@H]4CC[C@H](CN32)N4)C1] | (5aS,6S,9R)-3-chloro-2-(6-fluoro- 1-methyl-1H-indazol-7-yl)-13- (((S)-2-methylenetetrahydro-1H- pyrrolizin-7a(5H)-yl)methoxy)- 5a,6,7,8,9,10-hexahydro-5H-6,9- epiminoazepino[2′, 1′:3,4][1,4] oxazepino[5,6,7-de]quinazoline | 602 |
| 17 | 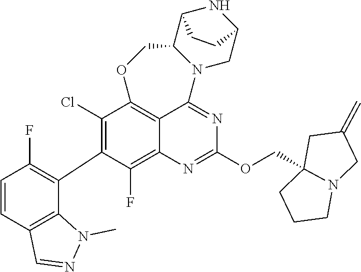 [SMILES: C=C1CN2CCC[C@@]2(COc2nc3c4c(c(Cl)c(-c5c(F)ccc6cnn(C)c56)c(F)c4n2)OC[C@@H]2[C@@H]4CC[C@H](CN32)N4)C1] | (5aS,6S,9R)-3-chloro-1-fluoro-2- (6-fluoro-1-methyl-1H-indazol-7- yl)-13-(((S)-2- methylenetetrahydro-1H- pyrrolizin-7a(5H)-yl)methoxy)- 5a,6,7,8,9,10-hexahydro-5H-6,9- epiminoazepino[2′, 1′:3,4][1,4] oxazepino[5,6,7-de]quinazoline | 620 |
| 17A | 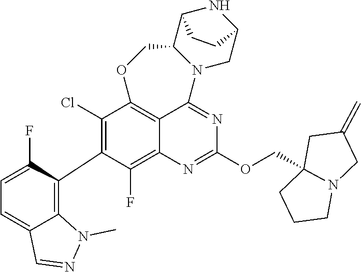 | | |
| 17B | 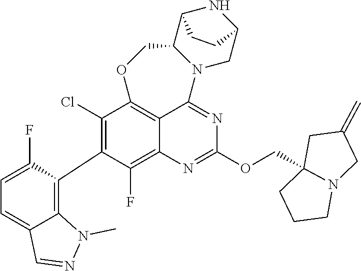 | | |
| 18 | 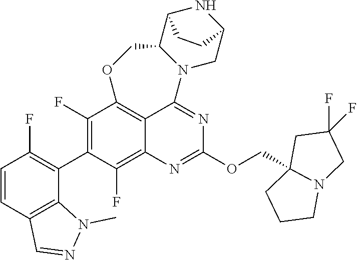 [SMILES: Cn1ncc2ccc(F)c(-c3c(F)c4c5c(nc(OC[C@@]67CCCN6CC(F)(F)C7)nc5c3F)N3C[C@H]5CC[C@H](N5)[C@H]3CO4)c21] | (5aS,6S,9R)-13-(((S)-2,2- difluorotetrahydro-1H-pyrrolizin- 7a(5H)-yl)methoxy)-1,3-difluoro- 2-(6-fluoro-1-methyl-1H-indazol- 7-yl)-5a,6,7,8,9,10-hexahydro- 5H-6,9- epiminoazepino[2′, 1′:3,4][1,4] oxazepino[5,6,7-de]quinazoline | 628 |
| 18A | 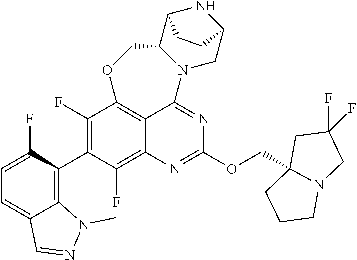 | | |
| 18B | 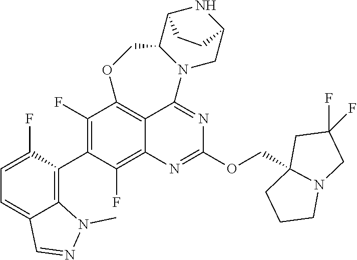 | | |
| 19 | 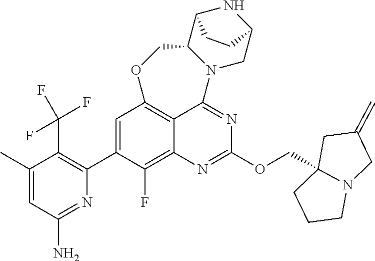 | 6-((5aS,6S,9R)-1-fluoro-13-(((S)- 2-methylenetetrahydro-1H- pyrrolizin-7a(5H)-yl)methoxy)- 5a,6,7,8,9,10-hexahydro-5H-6,9- epiminoazepino[2′, 1′:3,4][1,4] oxazepino[5,6,7-de]quinazolin- 2-yl)-4-methyl-5- (trifluoromethyl)pyridin-2-amine | 612 |
| 20 | 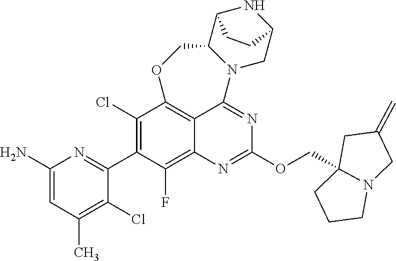 [SMILES: C=C1CN2CCC[C@@]2(COc2nc3c4c(c(Cl)c(-c5nc(N)cc(C)c5Cl)c(F)c4n2)OC[C@@H]2[C@@H]4CC[C@H](CN32)N4)C1] | 5-chloro-6-((5aS,6S,9R)-3- chloro-1-fluoro-13-(((S)-2- methylenetetrahydro-1H- pyrrolizin-7a(5H)-yl)methoxy)- 5a,6,7,8,9,10-hexahydro-5H-6,9- epiminoazepino[2′,1′:3,4][1,4] oxazepino[5,6,7-de]quinazolin- 2-yl)-4-methylpyridin-2-amine | 612 |
| 20A | 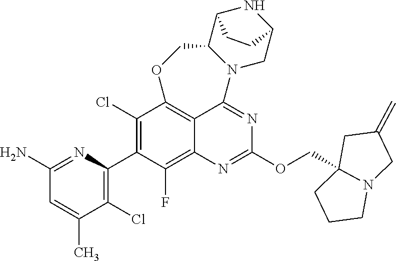 | | |
| 20B | 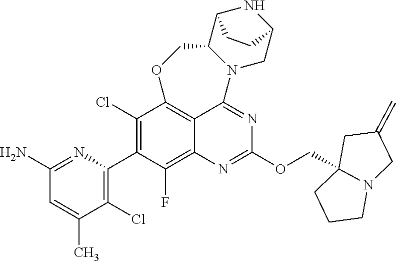 | | |
| 21 | 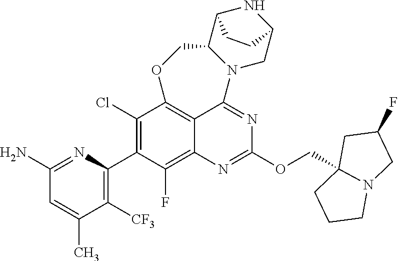 | 6-((2R,5aS,6S,9R)-3-chloro-1- fluoro-13-(((2R,7aS)-2- fluorotetrahydro-1H-pyrrolizin- 7a(5H)-yl)methoxy)- 5a,6,7,8,9,10-hexahydro-5H-6,9- epiminoazepino[2′, 1′:3,4][1,4] oxazepino[5,6,7-de]quinazolin- 2-yl)-4-methyl-5- (trifluoromethyl)pyridin-2-amine | 652 |
| 22 | 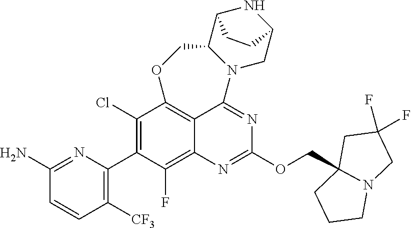 [SMILES: Nc1ccc(C(F)(F)F)c(-c2c(Cl)c3c4c(nc(OC[C@]56CCCN5CC(F)(F)C6)nc4c2F)N2C[C@H]4CC[C@H](N4)[C@H]2CO3)n1] | 6-((5aS,6S,9R)-3-chloro-13- (((R)-2,2-difluorotetrahydro-1H- pyrrolizin-7a(5H)-yl)methoxy)-1- fluoro-5a,6,7,8,9,10-hexahydro- 5H-6,9- epiminoazepino[2′,1′:3,4][1,4] oxazepino[5,6,7-de]quinazolin- 2-yl)-5-(trifluoromethyl)pyridin- 2-amine | 656 |
| 22A | 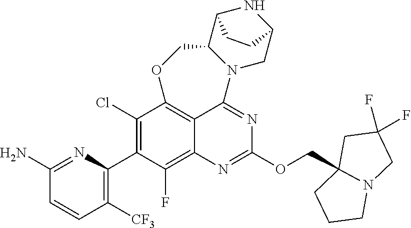 | | |
| 22B | 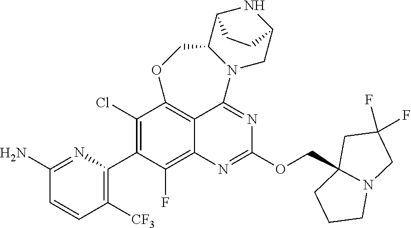 | | |
| 23 | 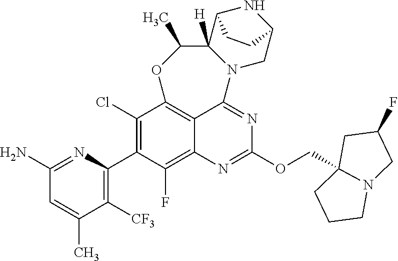 | 6-((2R,5S,5aS,6S,9R)-3-chloro- 1-fluoro-13-(((2R,7aS)-2- fluorotetrahydro-1H-pyrrolizin- 7a(5H)-yl)methoxy)-5-methyl- 5a,6,7,8,9,10-hexahydro-5H-6,9- epiminoazepino[2′,1′:3,4][1,4] oxazepino[5,6,7-de]quinazolin- 2-yl)-4-methyl-5- (trifluoromethyl)pyridin-2-amine | 666 |
| 24 | 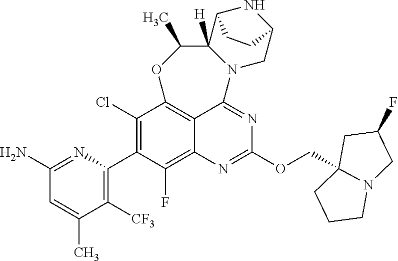 | 6-((2S,5S,5aS,6S,9R)-3-chloro- 1-fluoro-13-(((2R,7aS)-2- fluorotetrahydro-1H-pyrrolizin- 7a(5H)-yl)methoxy)-5-methyl- 5a,6,7,8,9,10-hexahydro-5H-6,9- epiminoazepino[2′,1′:3,4][1,4] oxazepino[5,6,7-de]quinazolin- 2-yl)-4-methyl-5- (trifluoromethyl)pyridin-2-amine | 666 |
| 25 | 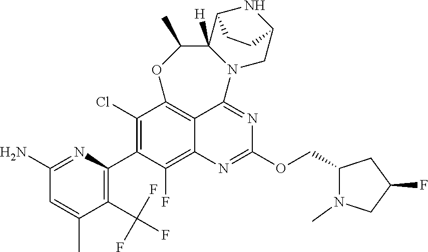 | 6-((2R,5S,5aS,6S,9R)-3-chloro- 1-fluoro-13-(((2S,4R)-4-fluoro-1- methylpyrrolidin-2-yl)methoxy)-5- methyl-5a,6,7,8,9,10-hexahydro- 5H-6,9- epiminoazepino[2′, 1′:3,4][1,4] oxazepino[5,6,7-de]quinazolin- 2-yl)-4-methyl-5- (trifluoromethyl)pyridin-2-amine | 640 |
| 26 | 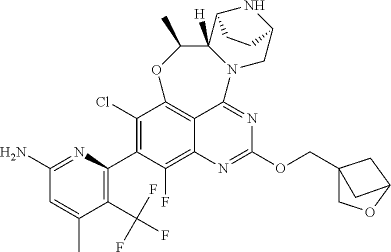 | 6-((2R,5S,5aS,6S,9R)-13-((2- oxabicyclo[2.1.1]hexan-4- yl)methoxy)-3-chloro-1-fluoro-5- methyl-5a,6,7,8,9,10-hexahydro- 5H-6,9- epiminoazepino[2′, 1′:3,4][1,4] oxazepino[5,6,7-de]quinazolin- 2-yl)-4-methyl-5- (trifluoromethyl)pyridin-2-amine | 621 |
| 27 | 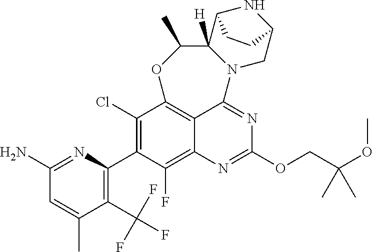 | 6-((2R,5S,5aS,6S,9R)-3-chloro- 1-fluoro-13-(2-methoxy-2- methylpropoxy)-5-methyl- 5a,6,7,8,9,10-hexahydro-5H-6,9- epiminoazepino[2′,1′:3,4][1,4] oxazepino[5,6,7-de]quinazolin- 2-yl)-4-methyl-5- (trifluoromethyl)pyridin-2-amine | 611 |
| 28 | 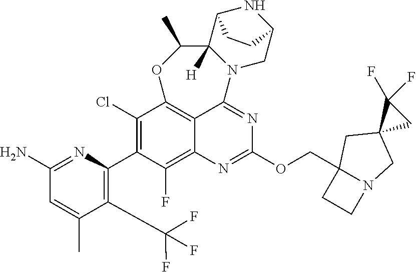 | 6-((2R,5S,5aS,6S,9R)-3-chloro- 13-(((3S)-2′,2′-difluoro-1- azaspiro[bicyclo[3.2.0]heptane- 3,1′-cyclopropan]-5-yl)methoxy)- 1-fluoro-5-methyl-5a,6,7,8,9,10- hexahydro-5H- [6,9]epiminoazepino[2′, 1′:3,4] [1,4]oxazepino[5,6,7- de]quinazolin- 2-yl)-4-methyl-5- (trifluoromethyl)pyridin-2-amine | 696 |
| 29 | 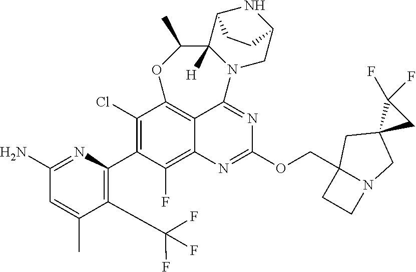 | 6-((2R,5S,5aS,6S,9R)-3-chloro- 13-(((3R)-2′,2′-difluoro-1- azaspiro[bicyclo[3.2.0]heptane- 3, 1′-cyclopropan]-5-yl)methoxy)- 1-fluoro-5-methyl-5a,6,7,8,9,10- hexahydro-5H- [6,9]epiminoazepino[2′,1′:3,4] [1,4]oxazepino[5,6,7-de] quinazolin-2-yl)-4-methyl-5- (trifluoromethyl)pyridin-2-amine | 696 |
| 30 | 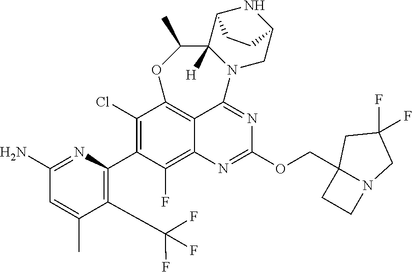 | 6-((2R,5S,5aS,6S,9R)-3-chloro- 13-((3,3-difluoro-1- azabicyclo[3.2.0]heptan-5- yl)methoxy)-1-fluoro-5-methyl- 5a,6,7,8,9,10-hexahydro-5H-6,9- epiminoazepino[2′,1′:3,4][1,4] oxazepino[5,6,7-de] quinazolin-2-yl)-4-methyl-5- (trifluoromethyl)pyridin-2-amine | 670 |
| 30A | 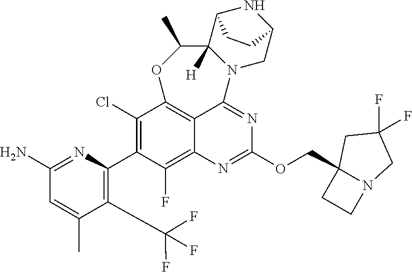 | | |
| 30B | 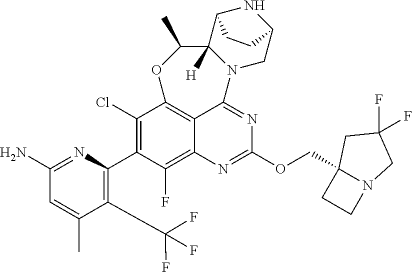 | | |
| 31 | 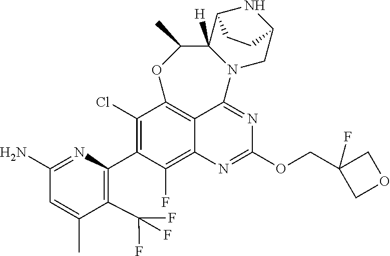 | 6-((2R,5S,5aS,6S,9R)-3-chloro- 1-fluoro-13-((3-fluorooxetan-3- yl)methoxy)-5-methyl- 5a,6,7,8,9,10-hexahydro-5H-6,9- epiminoazepino[2′,1′:3,4][1,4] oxazepino[5,6,7-de]quinazolin- 2-yl)-4-methyl-5- (trifluoromethyl)pyridin-2-amine | 613 |
| 32 | 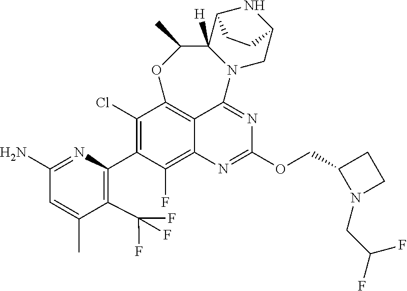 | 6-((2R,5S,5aS,6S,9R)-3-chloro- 13-(((S)-1-(2,2- difluoroethyl)azetidin-2- yl)methoxy)-1-fluoro-5-methyl- 5a,6,7,8,9,10-hexahydro-5H-6,9- epiminoazepino[2′, 1′:3,4][1,4] oxazepino[5,6,7-de]quinazolin- 2-yl)-4-methyl-5- (trifluoromethyl)pyridin-2-amine | 658 |
| 33 | 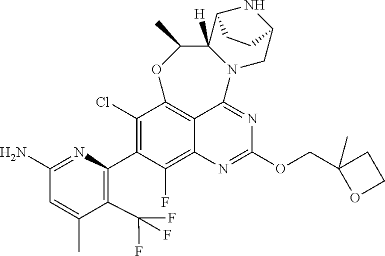 | 6-((2R,5S,5aS,6S,9R)-3-chloro- 1-fluoro-5-methyl-13-((2- methyloxetan-2-yl)methoxy)- 5a,6,7,8,9,10-hexahydro-5H-6,9- epiminoazepino[2′,1′:3,4][1,4] oxazepino[5,6,7-de]quinazolin- 2-yl)-4-methyl-5- (trifluoromethyl)pyridin-2-amine | 609 |
| 34 | 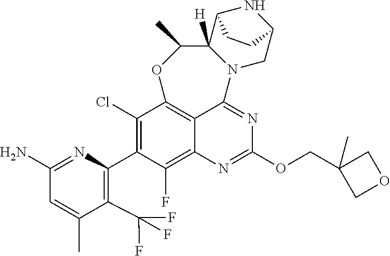 | 6-((2R,5S,5aS,6S,9R)-3-chloro- 1-fluoro-5-methyl-13-((3- methyloxetan-3-yl)methoxy)- 5a,6,7,8,9,10-hexahydro-5H-6,9- epiminoazepino[2′,1′:3,4][1,4] oxazepino[5,6,7-de]quinazolin- 2-yl)-4-methyl-5- (trifluoromethyl)pyridin-2-amine | 609 |
| 35 | 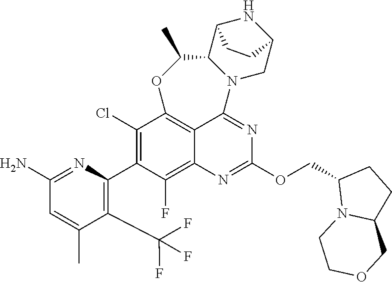 | 6-((2R,5S,5aS,6S,9R)-3-chloro- 1-fluoro-13-(((6S,8aS)- hexahydro-1H-pyrrolo[2,1- c][1,4]oxazin-6-yl)methoxy)-5- methyl-5a,6,7,8,9,10-hexahydro- 5H-6,9- epiminoazepino[2′, 1′:3,4][1,4] oxazepino[5,6,7-de] quinazolin-2-yl)-4-methyl-5- (trifluoromethyl)pyridin-2-amine | 664 |
| 36 | 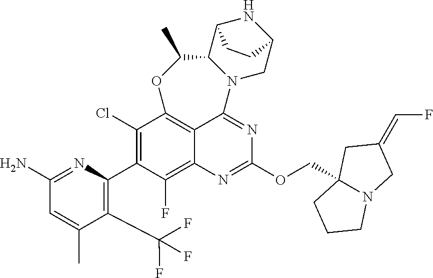 | 6-((2R,5S,5aS,6S,9R)-3-chloro- 1-fluoro-13-(((S,Z)-2- (fluoromethylene)tetrahydro-1H- pyrrolizin-7a(5H)-yl)methoxy)-5- methyl-5a,6,7,8,9,10-hexahydro- 5H-6,9-epiminoazepino [2′,1′:3,4][1,4]oxazepino [5,6,7-de]quinazolin-2-yl)- 4-methyl-5- (trifluoromethyl)pyridin-2-amine | 678 |
| 37 | 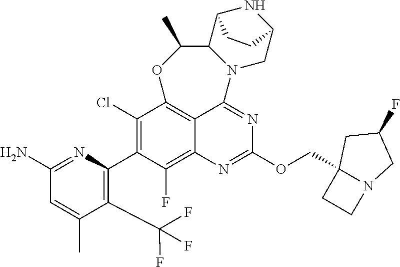 | 6-((2R,5S,5aS,6S,9R)-3-chloro- 1-fluoro-13-(((3R)-3-fluoro-1- azabicyclo[3.2.0]heptan-5- yl)methoxy)-5-methyl- 5a,6,7,8,9,10-hexahydro-5H-6,9- epiminoazepino[2′, 1′:3,4][1,4] oxazepino[5,6,7-de]quinazolin- 2-yl)-4-methyl-5- (trifluoromethyl)pyridin-2-amine | 652 |
| 38A | 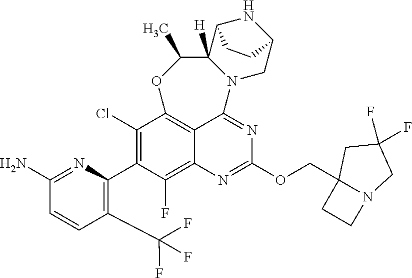 | 6-((2R,5S,5aS,6S,9R)-3-chloro- 13-((3,3-difluoro-1- azabicyclo[3.2.0]heptan-5- yl)methoxy)-1-fluoro-5-methyl- 5a,6,7,8,9,10-hexahydro-5H-6,9- epiminoazepino[2′, 1′:3,4][1,4] oxazepino[5,6,7-de]quinazolin- 2-yl)-5-(trifluoromethyl) pyridin-2-amine | 656 |
| | 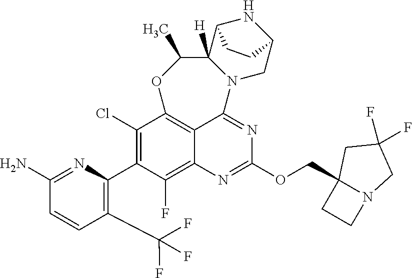 | | |
| | 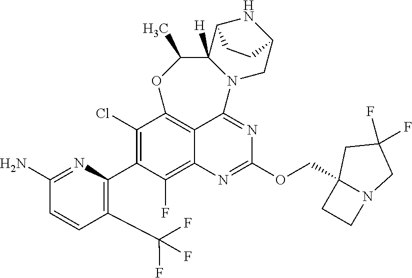 | | |
| 39 | 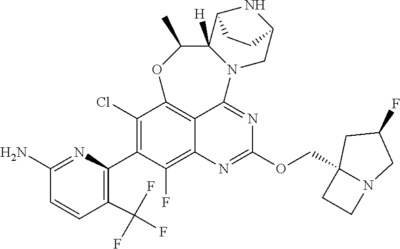 | 6-((2R,5S,5aS,6S,9R)-3-chloro- 1-fluoro-13-(((2R,7aS)-2- fluorotetrahydro-1H-pyrrolizin- 7a(5H)-yl)methoxy)-5-methyl- 5a,6,7,8,9,10-hexahydro-5H-6,9- epiminoazepino[2′, 1′:3,4][1,4] oxazepino[5,6,7-de]quinazolin-2-yl)- 5-(trifluoromethyl)pyridin-2-amine | 652 |
| 40 | 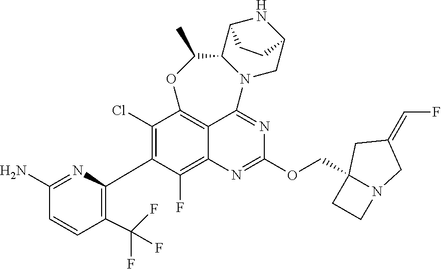 | 6-((2R,5S,5aS,6S,9R)-3-chloro- 1-fluoro-13-(((S,Z)-2- (fluoromethylene)tetrahydro-1H- pyrrolizin-7a(5H)-yl)methoxy)-5- methyl-5a,6,7,8,9,10-hexahydro- 5H-6,9-epiminoazepino [2′, 1′:3,4][1,4]oxazepino [5,6,7-de]quinazolin-2-yl)- 5-(trifluoromethyl)pyridin-2-amine | 664 |
| 41 | 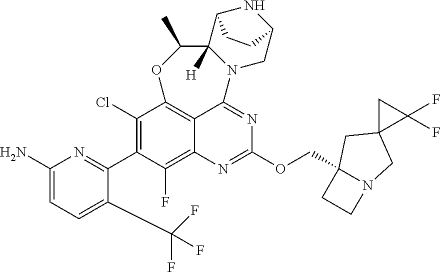 | 6-((5S,5aS,6S,9R)-3-chloro-13- (((7a′S)-2,2-difluorodihydro- 1′H,3′H-spiro[cyclopropane-1,2′- pyrrolizin]-7a′(5′H)-yl)methoxy)- 1-fluoro-5-methyl-5a,6,7,8,9,10- hexahydro-5H- [6,9]epiminoazepino[2′,1′:3,4] [1,4]oxazepino[5,6,7-de]quinazolin- 2-yl)-5-(trifluoromethyl)pyridin-2- amine | 696 |
| 41A | 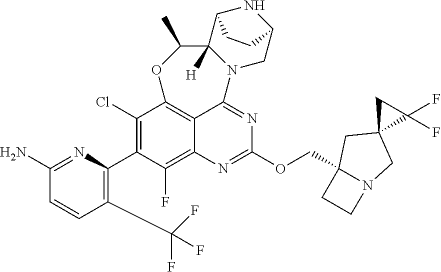 | | |
| 41B | 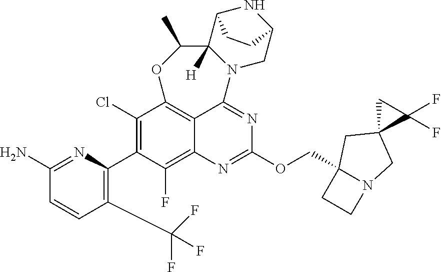 | | |
| 42 | 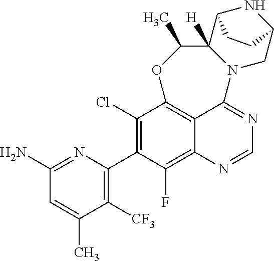 [SMILES: Cc1cc(N)nc(-c2c(Cl)c3c4c(ncnc4c2F)N2C[C@H]4CC[C@H](N4)[C@H]2[C@H](C)O3)c1C(F)(F)F] | 6-((5S,5aS,6S,9R)-3-Chloro-1- fluoro-5-methyl-5a,6,7,8,9,10- hexahydro-5H-6,9- epiminoazepino[2′,1′:3,4][1,4] oxazepino[5,6,7-de]quinazolin- 2-yl)-4-methyl-5- (trifluoromethyl)pyridin-2-amine | 509.1 |
| 42A | 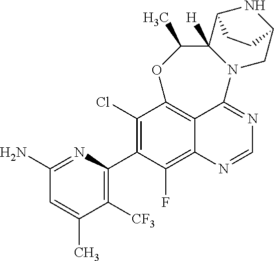 | | |
| 42B | 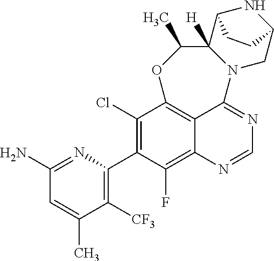 | | |
| 43 | 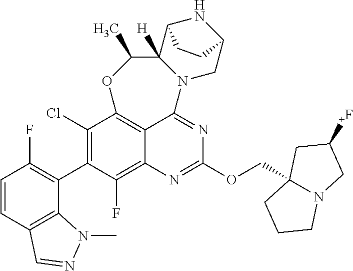 [SMILES: C[C@@H]1Oc2c(Cl)c(-c3c(F)ccc4cnn(C)c34)c(F)c3nc(OC[C@@]45CCCN4C[C@H]([FH+])C5)nc(c23)N2C[C@H]3CC[C@H](N3)[C@@H]12] | (5S,5aS,6S,9R)-3-Chloro-1- fluoro-2-(6-fluoro-1-methyl-1H- indazol-7-yl)-13-(((2R,7aS)-2- fluorotetrahydro-1H-pyrrolizin- 7a(5H)-yl)methoxy)-5-methyl- 5a,6,7,8,9,10-hexahydro-5H-6,9- epiminoazepino[2′,1′:3,4][1,4] oxazepino[5,6,7-de]quinazoline | 640 |
| 43A | 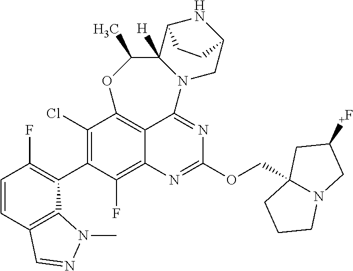 | | |
| 43B | 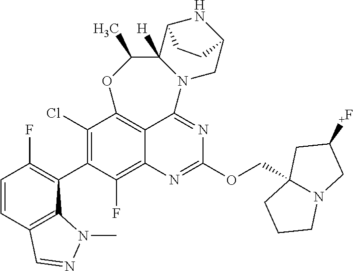 | | |
| 44 | 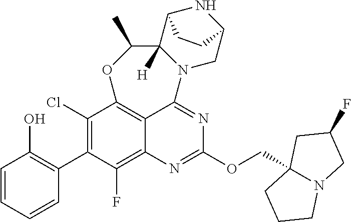 | 2-((5S,5aS,6S,9R)-3-Chloro-1- fluoro-13-(((2R,7aS)-2- fluorotetrahydro-1H-pyrrolizin- 7a(5H)-yl)methoxy)-5-methyl- 5a,6,7,8,9,10-hexahydro-5H-6,9- epiminoazepino[2′,1′:3,4][1,4] oxazepino[5,6,7-de]quinazolin-2- yl)phenol | 584 |

In one embodiment, the compound of formula (I) or a stereoisomer, atropisomer, tautomer, or pharmaceutically acceptable salt thereof is a compound of Table 2.

TABLE 2

| Cmpd | | | MS |
|---|---|---|---|
| No | Structure | Chemical Name | (M + H) |
| 45 45A 45B | 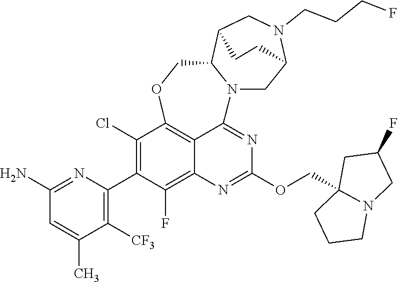 [SMILES: Cc1cc(N)nc(-c2c(Cl)c3c4c(nc(OC[C@@]56CCCN5C[C@H](F)C6)nc4c2F)N2C[C@H]4CC[C@H](CN4CCCF)[C@H]2CO3)c1C(F)(F)F] | 6-((5aS,6R,9R)-3-Chloro-1-fluoro-15-(3-fluoropropyl)- 13-(((2R,7aS)-2-fluorotetrahydro-1H-pyrrolizin-7a(5H)- yl)methoxy)-5a,6,7,8,9,10-hexahydro-5H-9,6- (epiminomethano)azepino[2′,1′:3,4][1,4]oxazepino[5,6,7- de]quinazolin-2-yl)-4-methyl-5-(trifluoromethyl)pyridin- 2-amine | 726 |
| | 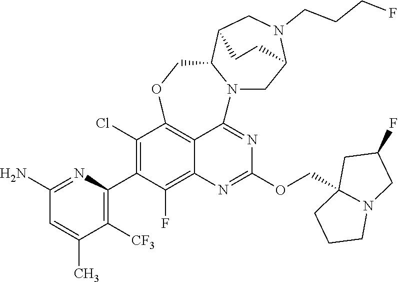 | | |
| | 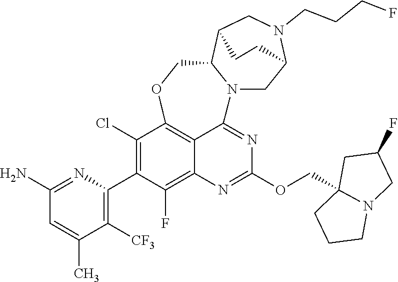 | | |
| 46 | 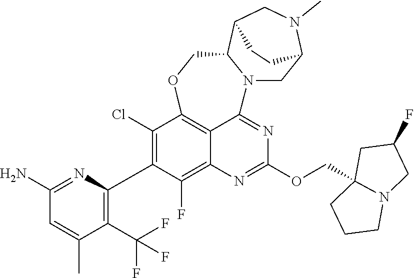 | 6-((2R,5aS,6R,9R)-3-Chloro-1-fluoro-13-(((2R,7aS)-2- fluorotetrahydro-1H-pyrrolizin-7a(5H)-yl)methoxy)-15- methyl-5a,6,7,8,9,10-hexahydro-5H-9,6- (epiminomethano)azepino[2′,1′:3,4][1,4]oxazepino[5,6,7- de]quinazolin-2-yl)-4-methyl-5-(trifluoromethyl)pyridin- 2-amine | 680 |
| 47 | 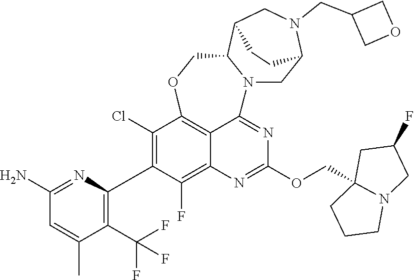 | 6-((2R,5aS,6R,9R)-3-Chloro-1-fluoro-13-(((2R,7aS)-2- fluorotetrahydro-1H-pyrrolizin-7a(5H)-yl)methoxy)-15- (oxetan-3-ylmethyl)-5a,6,7,8,9,10-hexahydro-5H-9,6- (epiminomethano)azepino[2′,1′:3,4][1,4]oxazepino[5,6,7- de]quinazolin-2-yl)-4-methyl-5-(trifluoromethyl)pyridin- 2-amine | 736 |
| 48 | 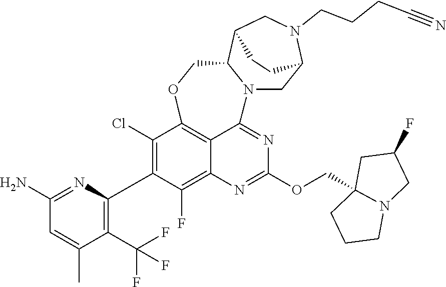 | 4-((2R,5aS,6R,9R)-2-(6-Amino-4-methyl-3- (trifluoromethyl)pyridin-2-yl)-3-chloro-1-fluoro-13- (((2R,7aS)-2-fluorotetrahydro-1H-pyrrolizin-7a(5H)- yl)methoxy)-5a,6,7,8,9,10-hexahydro-5H-9,6- (epiminomethano)azepino[2′,1′:3,4][1,4]oxazepino[5,6,7- de]quinazolin-15-yl)butanenitrile | 733 |
| 49 | 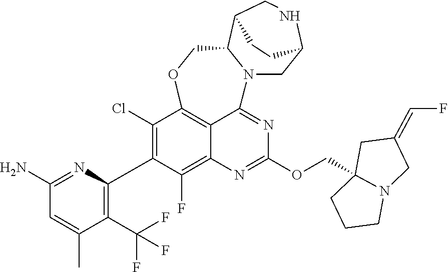 | 6-((2R,5aS,6R,9R)-3-Chloro-1-fluoro-13-(((S,Z)-2- (fluoromethylene)tetrahydro-1H-pyrrolizin-7a(5H)- yl)methoxy)-5a,6,7,8,9,10-hexahydro-5H-9,6- (epiminomethano)azepino[2′,1′:3,4][1,4]oxazepino[5,6,7- de]quinazolin-2-yl)-4-methyl-5-(trifluoromethyl)pyridin- 2-amine | 678 |
| 50 | 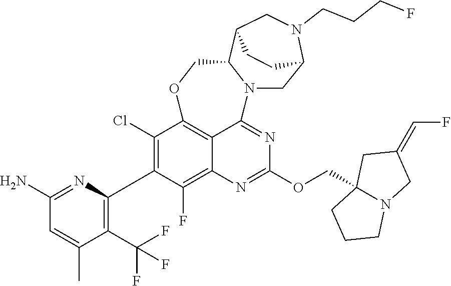 | 6-((2R,5aS,6R,9R)-3-Chloro-1-fluoro-13-(((S,Z)-2- (fluoromethylene)tetrahydro-1H-pyrrolizin-7a(5H)- yl)methoxy)-15-(3-fluoropropyl)-5a,6,7,8,9,10- hexahydro-5H-9,6- (epiminomethano)azepino[2′,1′:3,4][1,4]oxazepino[5,6,7- de]quinazolin-2-yl)-4-methyl-5-(trifluoromethyl)pyridin- 2-amine | 738 |
| 51 51A 51B | 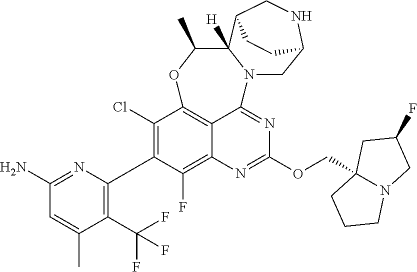 [SMILES: Cc1cc(N)nc(-c2c(Cl)c3c4c(nc(OC[C@@]56CCCN5C[C@H](F)C6)nc4c2F)N2C[C@H]4CC[C@H](CN4)[C@H]2[C@H](C)O3)c1C(F)(F)F] | 6-((5S,5aS,6R,9R)-3-Chloro-1-fluoro-13-(((2R,7aS)-2- fluorotetrahydro-1H-pyrrolizin-7a(5H)-yl)methoxy)-5- methyl-5a,6,7,8,9,10-hexahydro-5H-9,6- (epiminomethano)azepino[2′,1′:3,4][1,4]oxazepino[5,6,7- de]quinazolin-2-yl)-4-methyl-5-(trifluoromethyl)pyridin- 2-amine | 915 |
| | 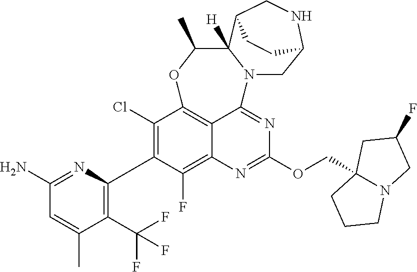 | | |
| | 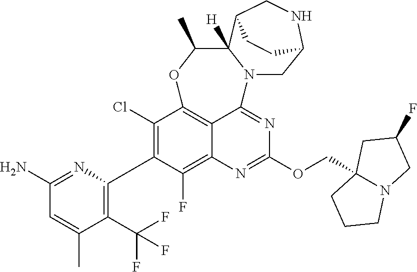 | | |
| 52 52A 52B | 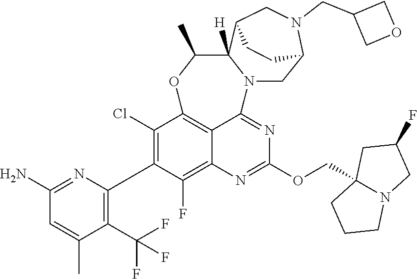 [SMILES: Cc1cc(N)nc(-c2c(Cl)c3c4c(nc(OC[C@@]56CCCN5C[C@H](F)C6)nc4c2F)N2C[C@H]4CC[C@H](CN4CC4COC4)[C@H]2[C@H](C)O3)c1C(F)(F)F] | 6-((5S,5aS,6R,9R)-3-Chloro-1-fluoro-13-(((2R,7aS)-2- fluorotetrahydro-1H-pyrrolizin-7a(5H)-yl)methoxy)-5- methyl-15-(oxetan-3-ylmethyl)-5a,6,7,8,9,10-hexahydro- 5H-9,6- (epiminomethano)azepino[2′,1′:3,4][1,4]oxazepino[5,6,7- de]quinazolin-2-yl)-4-methyl-5-(trifluoromethyl)pyridin- 2-amine | 750 |
| | 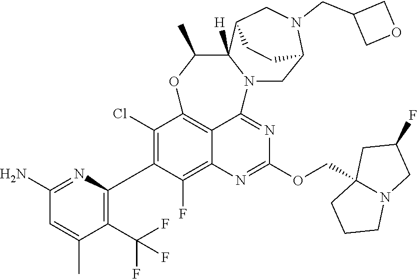 | | |
| | 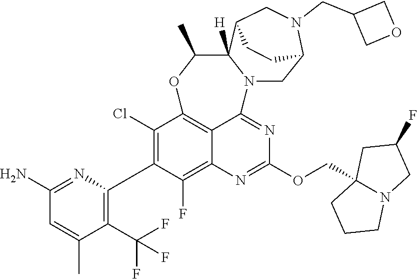 | | |

In one embodiment, the compound of formula (I) or a stereoisomer, atropisomer, tautomer, or pharmaceutically acceptable salt thereof is a compound of Table 3.

TABLE 3

| Cmpd | | |
|---|---|---|
| No | Structure | Chemical Name |
| 501A | 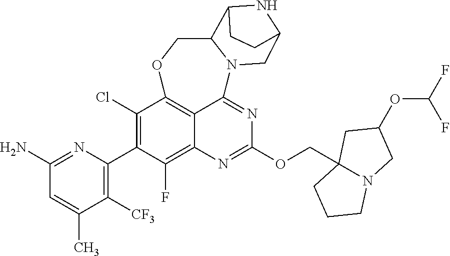 [SMILES: Cc1cc(N)nc(-c2c(Cl)c3c4c(nc(OCC56CCCN5CC(OC(F)F)C6)nc4c2F)N2CC4CCC(N4)C2CO3)c1C(F)(F)F] | 6-(3-chloro-13-((2-(difluoromethoxy)tetrahydro-1H- pyrrolizin-7a(5H)-yl)methoxy)-1-fluoro-5a,6,7,8,9,10- hexahydro-5H-6,9- epiminoazepino[2′,1′:3,4][1,4]oxazepino[5,6,7- de]quinazolin-2-yl)-4-methyl-5-(trifluoromethyl)pyridin- 2-amine |
| 501B | 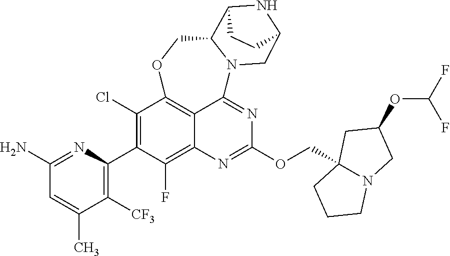 | 6-((2R,5aS,6S,9R)-3-chloro-13-(((2R,7aS)-2- (difluoromethoxy)tetrahydro-1H-pyrrolizin-7a(5H)- yl)methoxy)-1-fluoro-5a,6,7,8,9,10-hexahydro-5H-6,9- epiminoazepino[2′,1′:3,4][1,4]oxazepino[5,6,7- de]quinazolin-2-yl)-4-methyl-5-(trifluoromethyl)pyridin- 2-amine |
| 502A | 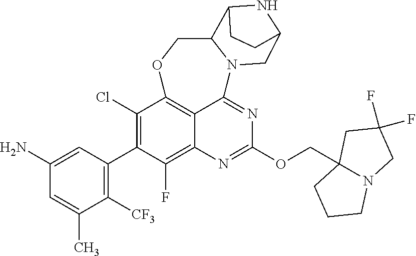 [SMILES: Cc1cc(N)cc(-c2c(Cl)c3c4c(nc(OCC56CCCN5CC(F)(F)C6)nc4c2F)N2CC4CCC(N4)C2CO3)c1C(F)(F)F] | 3-(3-chloro-13-((2,2-difluorotetrahydro-1H-pyrrolizin- 7a(5H)-yl)methoxy)-1-fluoro-5a,6,7,8,9,10-hexahydro- 5H-6,9-epiminoazepino[2′,1′:3,4][1,4]oxazepino[5,6,7- de]quinazolin-2-yl)-5-methyl-4-(trifluoromethyl)aniline |
| 502B | 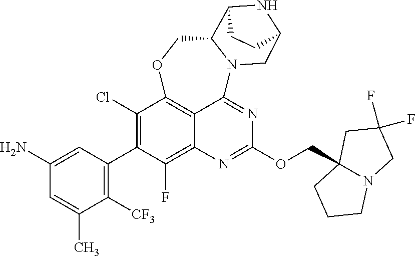 [SMILES: Cc1cc(N)cc(-c2c(Cl)c3c4c(nc(OC[C@]56CCCN5CC(F)(F)C6)nc4c2F)N2C[C@H]4CC[C@H](N4)[C@H]2CO3)c1C(F)(F)F] | 3-((5aS,6S,9R)-3-chloro-13-(((R)-2,2-difluorotetrahydro- 1H-pyrrolizin-7a(5H)-yl)methoxy)-1-fluoro- 5a,6,7,8,9,10-hexahydro-5H-6,9- epiminoazepino[2′,1′:3,4][1,4]oxazepino[5,6,7- de]quinazolin-2-yl)-5-methyl-4-(trifluoromethyl)aniline |
| 503A | 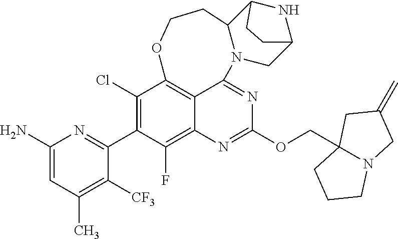 [SMILES: C=C1CN2CCCC2(COc2nc3c4c(c(Cl)c(-c5nc(N)cc(C)c5C(F)(F)F)c(F)c4n2)OCCC2C4CCC(CN32)N4)C1] | 6-(3-chloro-1-fluoro-14-((2-methylenetetrahydro-1H- pyrrolizin-7a(5H)-yl)methoxy)-5,6,6a,7,8,9,10,11- octahydro-7,10- epiminoazepino[1′,2′:5,6][1,5]oxazocino[4,3,2- de]quinazolin-2-yl)-4-methyl-5-(trifluoromethyl)pyridin- 2-amine |
| 503B | 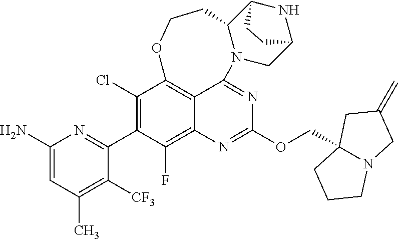 [SMILES: C=C1CN2CCC[C@@]2(COc2nc3c4c(c(Cl)c(-c5nc(N)cc(C)c5C(F)(F)F)c(F)c4n2)OCC[C@@H]2[C@@H]4CC[C@H](CN32)N4)C1] | 6-((6aR,7S, 10R)-3-chloro-1-fluoro-14-(((S)-2- methylenetetrahydro-1H-pyrrolizin-7a(5H)-yl)methoxy)- 5,6,6a,7,8,9,10,11-octahydro-7,10- epiminoazepino[1′,2′:5,6][1,5]oxazocino[4,3,2- de]quinazolin-2-yl)-4-methyl-5-(trifluoromethyl)pyridin- 2-amine |
| 504A | 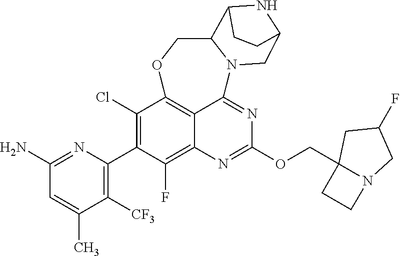 [SMILES: Cc1cc(N)nc(-c2c(Cl)c3c4c(nc(OCC56CCN5CC(F)C6)nc4c2F)N2CC4CCC(N4)C2CO3)c1C(F)(F)F] | 6-(3-chloro-1-fluoro-13-((3-fluoro-1- azabicyclo[3.2.0]heptan-5-yl)methoxy)-5a,6,7,8,9,10- hexahydro-5H-6,9- epiminoazepino[2′,1′:3,4][1,4]oxazepino[5,6,7- de]quinazolin-2-yl)-4-methyl-5-(trifluoromethyl)pyridin- 2-amine |
| 504B | 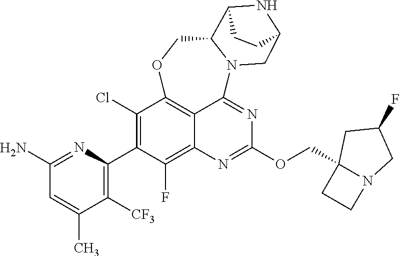 | 6-((2R,5aS,6S,9R)-3-chloro-1-fluoro-13-(((3R,5S)-3- fluoro-1-azabicyclo[3.2.0]heptan-5-yl)methoxy)- 5a,6,7,8,9,10-hexahydro-5H-6,9- epiminoazepino[2′,1′:3,4][1,4]oxazepino[5,6,7- de]quinazolin-2-yl)-4-methyl-5-(trifluoromethyl)pyridin- 2-amine |
| 505A | 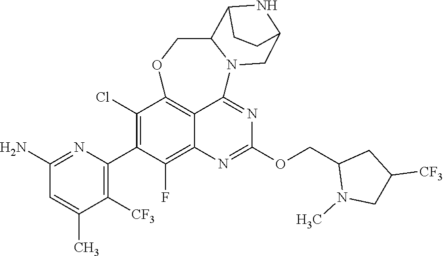 [SMILES: Cc1cc(N)nc(-c2c(Cl)c3c4c(nc(OCC5CC(C(F)(F)F)CN5C)nc4c2F)N2CC4CCC(N4)C2CO3)c1C(F)(F)F] | 6-(3-chloro-1-fluoro-13-((1-methyl-4- (trifluoromethyl)pyrrolidin-2-yl)methoxy)-5a,6,7,8,9,10- hexahydro-5H-6,9- epiminoazepino[2′,1′:3,4][1,4]oxazepino[5,6,7- de]quinazolin-2-yl)-4-methyl-5-(trifluoromethyl)pyridin- 2-amine |
| 505B | 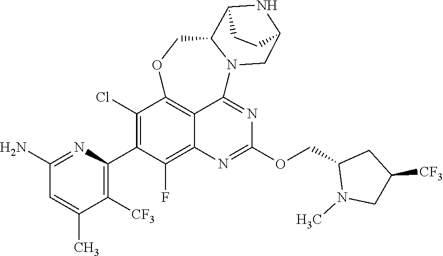 | 6-((2R,5aS,6S,9R)-3-chloro-1-fluoro-13-(((2S,4R)-1- methyl-4-(trifluoromethyl)pyrrolidin-2-yl)methoxy)- 5a,6,7,8,9,10-hexahydro-5H-6,9- epiminoazepino[2′,1′:3,4][1,4]oxazepino[5,6,7- de]quinazolin-2-yl)-4-methyl-5-(trifluoromethyl)pyridin- 2-amine |
| 506A | 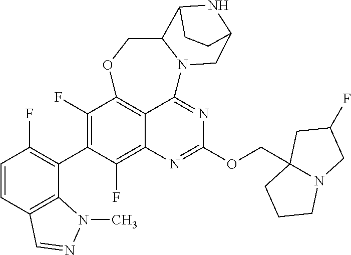 [SMILES: Cn1ncc2ccc(F)c(-c3c(F)c4c5c(nc(OCC67CCCN6CC(F)C7)nc5c3F)N3CC5CCC(N5)C3CO4)c21] | 1,3-difluoro-2-(6-fluoro-1-methyl-1H-indazol-7-yl)-13- ((2-fluorotetrahydro-1H-pyrrolizin-7a(5H)-yl)methoxy)- 5a,6,7,8,9,10-hexahydro-5H-6,9- epiminoazepino[2′,1′:3,4][1,4]oxazepino[5,6,7- de]quinazoline |
| 506B | 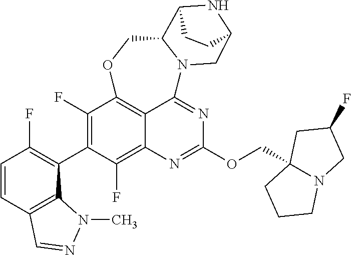 | (2S,5aS,6S,9R)-1,3-difluoro-2-(6-fluoro-1-methyl-1H- indazol-7-yl)-13-(((2R,7aS)-2-fluorotetrahydro-1H- pyrrolizin-7a(5H)-yl)methoxy)-5a,6,7,8,9,10-hexahydro- 5H-6,9-epiminoazepino[2′, 1′:3,4][1,4]oxazepino[5,6,7- de]quinazoline |
| 507A | 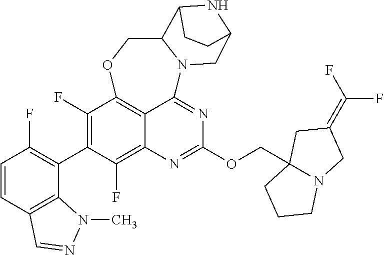 [SMILES: Cn1ncc2ccc(F)c(-c3c(F)c4c5c(nc(OCC67CCCN6CC(=C(F)F)C7)nc5c3F)N3CC5CCC(N5)C3CO4)c21] | 13-((2-(difluoromethylene)tetrahydro-1H-pyrrolizin- 7a(5H)-yl)methoxy)-1,3-difluoro-2-(6-fluoro-1-methyl- 1H-indazol-7-yl)-5a,6,7,8,9,10-hexahydro-5H-6,9- epiminoazepino[2′,1′:3,4][1,4]oxazepino[5,6,7- de]quinazoline |
| 507B | 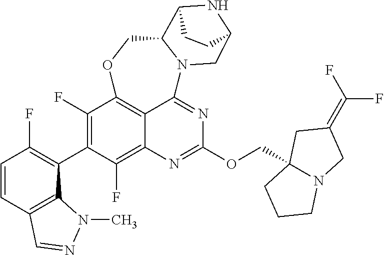 | (2S,5aS,6S,9R)-13-(((S)-2- (difluoromethylene)tetrahydro-1H-pyrrolizin-7a(5H)- yl)methoxy)-1,3-difluoro-2-(6-fluoro-1-methyl-1H- indazol-7-yl)-5a,6,7,8,9,10-hexahydro-5H-6,9- epiminoazepino[2′,1′:3,4][1,4]oxazepino[5,6,7- de]quinazoline |
| 508A | 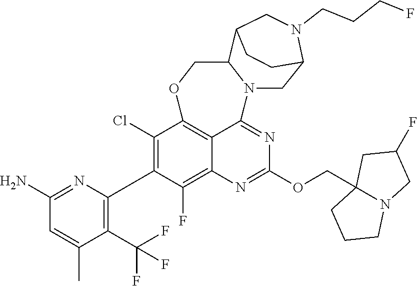 [SMILES: Cc1cc(N)nc(-c2c(Cl)c3c4c(nc(OCC56CCCN5CC(F)C6)nc4c2F)N2CC4CCC(CN4CCCF)C2CO3)c1C(F)(F)F] | 6-(3-chloro-1-fluoro-15-(3-fluoropropyl)-13-((2- fluorotetrahydro-1H-pyrrolizin-7a(5H)-yl)methoxy)- 5a,6,7,8,9,10-hexahydro-5H-9,6- (epiminomethano)azepino[2′,1′:3,4][1,4]oxazepino[5,6,7- de]quinazolin-2-yl)-4-methyl-5-(trifluoromethyl)pyridin- 2-amine |
| 508B | 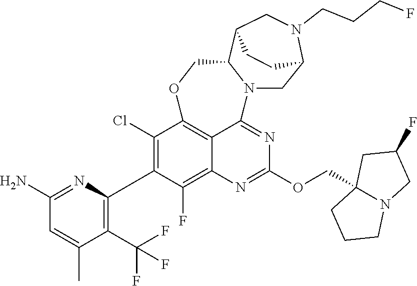 | 6-((2R,5aS,6R,9R)-3-chloro-1-fluoro-15-(3- fluoropropyl)-13-(((2R,7aS)-2-fluorotetrahydro-1H- pyrrolizin-7a(5H)-yl)methoxy)-5a,6,7,8,9,10-hexahydro- 5H-9,6- (epiminomethano)azepino[2′,1′:3,4][1,4]oxazepino[5,6,7- de]quinazolin-2-yl)-4-methyl-5-(trifluoromethyl)pyridin- 2-amine |
| 509A | 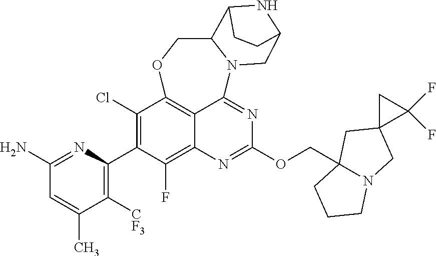 | 6-((2R)-3-chloro-13-((2,2-difluorodihydro-1′H,3′H- spiro[cyclopropane-1,2′-pyrrolizin]-7a′(5′H)- yl)methoxy)-1-fluoro-5a,6,7,8,9,10-hexahydro-5H- [6,9]epiminoazepino[2′,1′:3,4][1,4]oxazepino[5,6,7- de]quinazolin-2-yl)-4-methyl-5-(trifluoromethyl)pyridin- 2-amine |
| 509B | 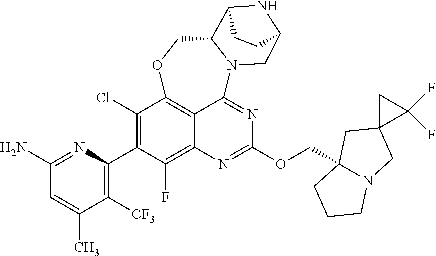 | 6-((2R,5aS,6S,9R)-3-chloro-13-(((7a′S)-2,2- difluorodihydro-1′H,3′H-spiro[cyclopropane-1,2′- pyrrolizin]-7a′(5′H)-yl)methoxy)-1-fluoro-5a,6,7,8,9,10- hexahydro-5H- [6,9]epiminoazepino[2′,1′:3,4][1,4]oxazepino[5,6,7- de]quinazolin-2-yl)-4-methyl-5-(trifluoromethyl)pyridin- 2-amine |
| 510A | 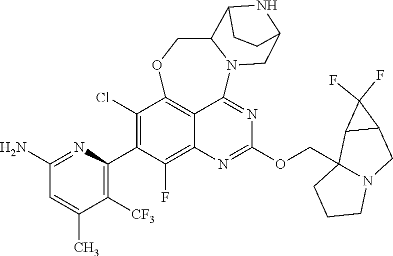 | 6-((2R)-3-chloro-13-((1,1- difluorohexahydrocyclopropa[a]pyrrolizin-6a(4H)- yl)methoxy)-1-fluoro-5a,6,7,8,9,10-hexahydro-5H-6,9- epiminoazepino[2′, 1′:3,4][1,4]oxazepino[5,6,7- de]quinazolin-2-yl)-4-methyl-5-(trifluoromethyl)pyridin- 2-amine |
| 510B | 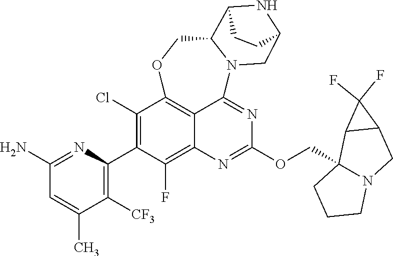 | 6-((2R,5aS,6S,9R)-3-chloro-13-(((6aS)-1,1- difluorohexahydrocyclopropa[a]pyrrolizin-6a(4H)- yl)methoxy)-1-fluoro-5a,6,7,8,9,10-hexahydro-5H-6,9- epiminoazepino[2′,1′:3,4][1,4]oxazepino[5,6,7- de]quinazolin-2-yl)-4-methyl-5-(trifluoromethyl)pyridin- 2-amine |
| 511A | 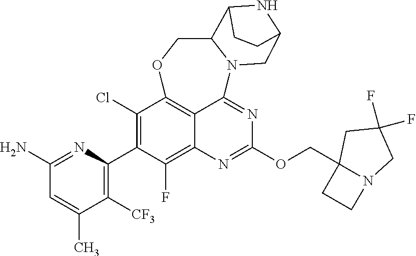 | 6-((2R)-3-chloro-13-((3,3-difluoro-1- azabicyclo[3.2.0]heptan-5-yl)methoxy)-1-fluoro- 5a,6,7,8,9,10-hexahydro-5H-6,9- epiminoazepino[2′,1′:3,4][1,4]oxazepino[5,6,7- de]quinazolin-2-yl)-4-methyl-5-(trifluoromethyl)pyridin- 2-amine |
| 511B | 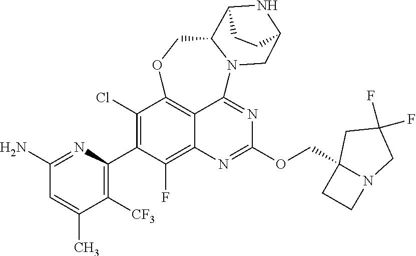 | 6-((2R,5aS,6S,9R)-3-chloro-13-(((S)-3,3-difluoro-1- azabicyclo[3.2.0]heptan-5-yl)methoxy)-1-fluoro- 5a,6,7,8,9,10-hexahydro-5H-6,9- epiminoazepino[2′,1′:3,4][1,4]oxazepino[5,6,7- de]quinazolin-2-yl)-4-methyl-5-(trifluoromethyl)pyridin- 2-amine |
| 512A | 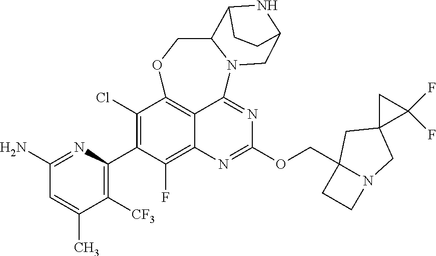 | 6-((2R)-3-chloro-13-((2′,2′-difluoro-1- azaspiro[bicyclo[3.2.0]heptane-3,1′-cyclopropan]-5- yl)methoxy)-1-fluoro-5a,6,7,8,9,10-hexahydro-5H- [6,9]epiminoazepino[2′,1′:3,4][1,4]oxazepino[5,6,7- de]quinazolin-2-yl)-4-methyl-5-(trifluoromethyl)pyridin- 2-amine |
| 512B | 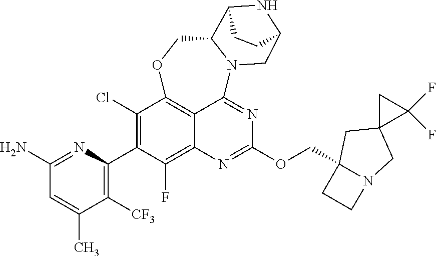 | 6-((2R,5aS,6S,9R)-3-chloro-13-(((5R)-2′,2′-difluoro-1- azaspiro[bicyclo[3.2.0]heptane-3,1′-cyclopropan]-5- yl)methoxy)-1-fluoro-5a,6,7,8,9,10-hexahydro-5H- [6,9]epiminoazepino[2′,1′:3,4][1,4]oxazepino[5,6,7- de]quinazolin-2-yl)-4-methyl-5-(trifluoromethyl)pyridin- 2-amine |
| 513A | 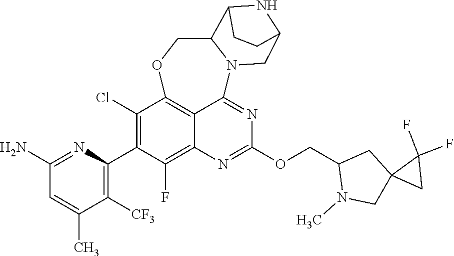 | 6-((2R)-3-chloro-13-((1,1-difluoro-5-methyl-5- azaspiro[2.4]heptan-6-yl)methoxy)-1-fluoro- 5a,6,7,8,9,10-hexahydro-5H- [6,9]epiminoazepino[2′,1′:3,4][1,4]oxazepino[5,6,7- de]quinazolin-2-yl)-4-methyl-5-(trifluoromethyl)pyridin- 2-amine |
| 513B | 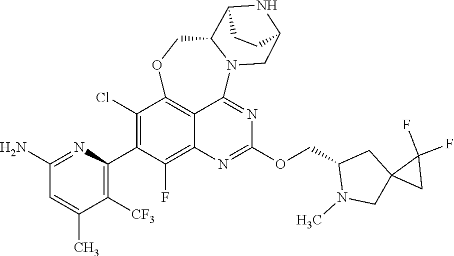 | 6-((2R,5aS,6S,9R)-3-chloro-13-(((6S)-1,1-difluoro-5- methyl-5-azaspiro[2.4]heptan-6-yl)methoxy)-1-fluoro- 5a,6,7,8,9,10-hexahydro-5H- [6,9]epiminoazepino[2′,1′:3,4][1,4]oxazepino[5,6,7- de]quinazolin-2-yl)-4-methyl-5-(trifluoromethyl)pyridin- 2-amine |
| 514A | 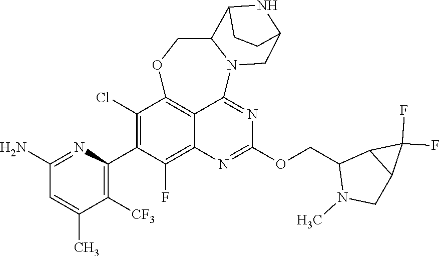 | 6-((2R)-3-chloro-13-((6,6-difluoro-3-methyl-3- azabicyclo[3.1.0]hexan-2-yl)methoxy)-1-fluoro- 5a,6,7,8,9,10-hexahydro-5H-6,9- epiminoazepino[2′,1′:3,4][1,4]oxazepino[5,6,7- de]quinazolin-2-yl)-4-methyl-5-(trifluoromethyl)pyridin- 2-amine |
| 514B | 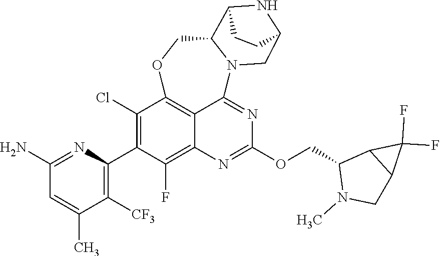 | 6-((2R,5aS,6S,9R)-3-chloro-13-(((2S)-6,6-difluoro-3- methyl-3-azabicyclo[3.1.0]hexan-2-yl)methoxy)-1- fluoro-5a,6,7,8,9,10-hexahydro-5H-6,9- epiminoazepino[2′,1′:3,4][1,4]oxazepino[5,6,7- de]quinazolin-2-yl)-4-methyl-5-(trifluoromethyl)pyridin- 2-amine |
| 515A | 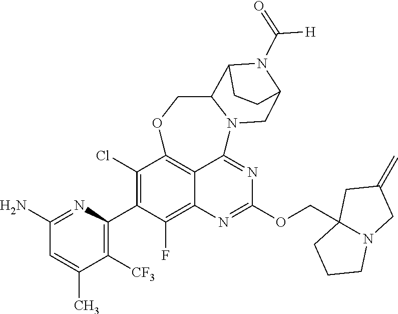 | (2R)-2-(6-amino-4-methyl-3-(trifluoromethyl)pyridin-2- yl)-3-chloro-1-fluoro-13-((2-methylenetetrahydro-1H- pyrrolizin-7a(5H)-yl)methoxy)-5a,6,7,8,9,10-hexahydro- 5H-6,9-epiminoazepino[2′,1′:3,4][1,4]oxazepino[5,6,7- de]quinazoline-15-carbaldehyde |
| 515B | 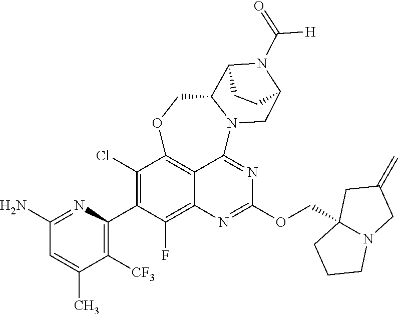 | (2R,5aS,6S,9R)-2-(6-amino-4-methyl-3- (trifluoromethyl)pyridin-2-yl)-3-chloro-1-fluoro-13- (((S)-2-methylenetetrahydro-1H-pyrrolizin-7a(5H)- yl)methoxy)-5a,6,7,8,9,10-hexahydro-5H-6,9- epiminoazepino[2′,1′:3,4][1,4]oxazepino[5,6,7- de]quinazoline-15-carbaldehyde |
| 516A | 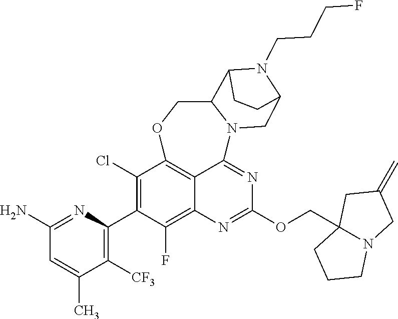 | 6-((2R)-3-chloro-1-fluoro-15-(3-fluoropropyl)-13-((2- methylenetetrahydro-1H-pyrrolizin-7a(5H)-yl)methoxy)- 5a,6,7,8,9,10-hexahydro-5H-6,9- epiminoazepino[2′,1′:3,4][1,4]oxazepino[5,6,7- de]quinazolin-2-yl)-4-methyl-5-(trifluoromethyl)pyridin- 2-amine |
| 516B | 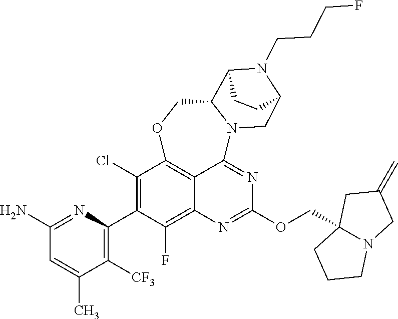 | 6-((2R,5aS,6S,9R)-3-chloro-1-fluoro-15-(3- fluoropropyl)-13-(((S)-2-methylenetetrahydro-1H- pyrrolizin-7a(5H)-yl)methoxy)-5a,6,7,8,9,10-hexahydro- 5H-6,9-epiminoazepino[2′,1′:3,4][1,4]oxazepino[5,6,7- de]quinazolin-2-yl)-4-methyl-5-(trifluoromethyl)pyridin- 2-amine |

### Synthesis of Compounds

Compounds or a stereoisomer, atropisomer, tautomer, or pharmaceutically acceptable salt thereof as described herein of the present disclosure can be made by a variety of methods depicted in the illustrative synthetic reaction schemes shown and described below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, vol. 1-21; R. C. LaRock, Comprehensive Organic Transformations, 2nd edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds.) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, vol. 1-40. The following synthetic reaction schemes are merely illustrative of some methods by which the compounds or pharmaceutical acceptable salts thereof described herein can be synthesized, and various modifications to these synthetic reaction schemes can be made and will be suggested to one skilled in the art having referred to the disclosure contained herein.

Synthetic chemistry transformations and protecting group methodologies (protection and deprotection) useful in synthesizing compounds described herein and necessary reagents and intermediates include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); and L. Paquette, ed., Encyclopedia of Reagents for Organic Synthesis, John Wiley and Sons (1995) and subsequent editions thereof.

Compounds or a stereoisomer, atropisomer, tautomer, or pharmaceutically acceptable salt thereof as described herein described herein can be prepared singly or as compound libraries comprising at least 2, for example 5 to 1,000 compounds, or 10 to 100 compounds. Libraries of compounds or a stereoisomer, atropisomer, tautomer, or pharmaceutically acceptable salt thereof as described herein of the formulae described herein can be prepared by a combinatorial split and mix approach or by multiple parallel syntheses using, for example, either solution phase or solid phase chemistry. Thus according to a further aspect provided herein is a compound library comprising at least 2 compounds or a stereoisomer, atropisomer, tautomer, or pharmaceutically acceptable salt thereof as described herein.

The Examples provide exemplary methods for preparing compounds or a stereoisomer, atropisomer, tautomer, or pharmaceutically acceptable salt thereof as described herein. Those skilled in the art will appreciate that other synthetic routes can be used to synthesize the compounds or a stereoisomer, atropisomer, tautomer, or pharmaceutically acceptable salt thereof as described herein described herein. Although specific starting materials and reagents are depicted and discussed in the Examples, other starting materials and reagents can be substituted to provide a variety of derivatives and/or reaction conditions. In addition, many of the exemplary compounds prepared by the described methods can be further modified in light of this disclosure using conventional chemistry.

In preparing compounds or a stereoisomer, atropisomer, tautomer, or pharmaceutically acceptable salt thereof as described herein protection of remote functionality (e.g., primary or secondary amine) of intermediates can be necessary. The need for such protection will vary depending on the nature of the remote functionality and the conditions of the preparation methods. Suitable amino-protecting groups include acetyl, trifluoroacetyl, t-butoxycarbonyl (BOC), benzyloxycarbonyl (CBz) and 9-fluorenylmethyleneoxycarbonyl (Fmoc). The need for such protection can be readily determined. For a general description of protecting groups and their use, see T. W. Greene, Protective Groups in Organic Synthesis, John Wiley & Sons, New York, 1991.

In the methods of preparing compounds or a stereoisomer, atropisomer, tautomer, or pharmaceutically acceptable salt thereof as described herein, it can be advantageous to separate reaction products from one another and/or from starting materials. The desired products of each step or series of steps is separated and/or purified to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (SMB) and preparative thin or thick layer chromatography, as well as techniques of small scale thin layer and flash chromatography.

Another class of separation methods involves treatment of a mixture with a reagent selected to bind to or render otherwise separable a desired product, unreacted starting material, reaction by product, or the like. Such reagents include adsorbents or absorbents such as activated carbon, molecular sieves, ion exchange media, or the like. Alternatively, the reagents can be acids in the case of a basic material, bases in the case of an acidic material, binding reagents such as antibodies, binding proteins, selective chelators such as crown ethers, liquid/liquid ion extraction reagents (LIX), or the like. Selection of appropriate methods of separation depends on the nature of the materials involved, such as, boiling point and molecular weight in distillation and sublimation, presence or absence of polar functional groups in chromatography, stability of materials in acidic and basic media in multiphase extraction, and the like.

Diastereomeric mixtures can be separated into their individual diastereomers on the basis of their physical chemical differences by methods such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Also, some of the compounds or a stereoisomer, atropisomer, tautomer, or pharmaceutically acceptable salt thereof as described herein described herein can be atropisomers (e.g., substituted biaryls). Enantiomers can also be separated by use of a chiral HPLC column.

A single stereoisomer, e.g., an enantiomer, substantially free of its stereoisomer can be obtained by resolution of the racemic mixture using a method such as formation of diastereomers using optically active resolving agents (Eliel, E. and Wilen, S. “Stereochemistry of Organic Compounds,” John Wiley & Sons, Inc., New York, 1994; Lochmuller, C. H., (1975) J. Chromatogr., 113(3):283-302). Racemic mixtures of chiral compounds or pharmaceutically acceptable salts thereof described herein can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: “Drug Stereochemistry, Analytical Methods and Pharmacology,” Irving W. Wainer, Ed., Marcel Dekker, Inc., New York (1993).

Under method (1), diastereomeric salts can be formed by reaction of enantiomerically pure chiral bases such as brucine, quinine, ephedrine, strychnine, α-methyl-p-phenylethylamine (amphetamine), and the like with asymmetric compounds bearing acidic functionality, such as carboxylic acid and sulfonic acid. The diastereomeric salts can be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts.

Alternatively, by method (2), the substrate to be resolved is reacted with one enantiomer of a chiral compound to form a diastereomeric pair (E. and Wilen, S. “Stereochemistry of Organic Compounds”, John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob III. J. Org. Chem. (1982) 47:4165), of the racemic mixture, and analyzing the ¹H NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111). By method (3), a racemic mixture of two enantiomers can be separated by chromatography using a chiral stationary phase (“Chiral Liquid Chromatography” (1989) W. J. Lough, Ed., Chapman and Hall, New York; Okamoto, J. Chromatogr., (1990) 513:375-378). Enriched or purified enantiomers can be distinguished by methods used to distinguish other chiral molecules with asymmetric carbon atoms, such as optical rotation and circular dichroism.

The chemical reactions described herein may be readily adapted to prepare other compounds and pharmaceutically acceptable salts thereof described herein. For example, the synthesis of non-exemplified compounds and pharmaceutically acceptable salts thereof described herein may be successfully performed by modifications apparent to those skilled in the art, e.g., by appropriately protecting interfering groups, by utilizing other suitable reagents known in the art other than those described, or by making routine modifications of reaction conditions. Alternatively, other reactions disclosed herein or known in the art will be recognized as having applicability for preparing other compounds and pharmaceutically acceptable salts thereof described herein.

### Pharmaceutical Formulations

Also provided herein are pharmaceutical compositions comprising compound or a stereoisomer, atropisomer, tautomer, or pharmaceutically acceptable salt thereof as described herein and one or more pharmaceutically acceptable excipients.

Compounds or a stereoisomer, atropisomer, tautomer, or pharmaceutically acceptable salt thereof as described herein as described herein can be formulated in accordance with standard pharmaceutical practice as a pharmaceutical composition. Thus, further provided herein is a pharmaceutical composition comprising a compound or a stereoisomer, atropisomer, tautomer, or pharmaceutically acceptable salt thereof as described herein as described herein and one or more pharmaceutically acceptable excipients.

A typical formulation is prepared by mixing a compound or pharmaceutically acceptable salt thereof as described herein and an excipient. Suitable carriers, diluents and excipients include, but are not limited to, materials such as carbohydrates, waxes, water soluble and/or swellable polymers, hydrophilic or hydrophobic materials, gelatin, oils, solvents, water and the like. The particular excipient used will depend upon the means and purpose for which the compound or pharmaceutically acceptable salt thereof as described herein is being applied. Solvents are generally selected based on solvents recognized as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations can also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound described herein or pharmaceutical composition thereof) or aid in the manufacturing of the pharmaceutical product (i.e., medicament).

The formulations can be prepared using conventional dissolution and mixing procedures. For example, the bulk drug substance (i.e., compound or pharmaceutically acceptable salt thereof as described herein or stabilized form thereof (e.g., complex with a cyclodextrin derivative or other known complexation agent) is dissolved in a suitable solvent in the presence of one or more of the excipients described above. The compound or a stereoisomer, atropisomer, tautomer, or pharmaceutically acceptable salt thereof as described herein as described herein is typically formulated into pharmaceutical dosage forms to provide an easily controllable dosage of the drug and to enable patient compliance with the prescribed regimen.

The pharmaceutical composition (or formulation) for application can be packaged in a variety of ways depending upon the method used for administering the drug. Generally, an article for distribution includes a container having deposited therein the pharmaceutical formulation in an appropriate form. Suitable containers include materials such as bottles (plastic and glass), sachets, ampoules, plastic bags, metal cylinders, and the like. The container can also include a tamper-proof assemblage to prevent indiscreet access to the contents of the package. In addition, the container has deposited thereon a label that describes the contents of the container. The label can also include appropriate warnings.

Pharmaceutical formulations of the compound or a stereoisomer, atropisomer, tautomer, or pharmaceutically acceptable salt thereof as described herein can be prepared for various routes and types of administration. For example, a compound or a stereoisomer, atropisomer, tautomer, or pharmaceutically acceptable salt thereof having the desired degree of purity can optionally be mixed with one or more pharmaceutically acceptable excipients (Remington's Pharmaceutical Sciences (1980) 16th edition, Osol, A. Ed.), in the form of a lyophilized formulation, milled powder, or an aqueous solution. Formulation can be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but can range from about 3 to about 8. For example, formulation in an acetate buffer at pH 5 can be a suitable embodiment.

The pharmaceutical composition ordinarily can be stored as a solid composition, a lyophilized formulation or as an aqueous solution.

The pharmaceutical compositions described herein can be formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The effective amount of the compound or a stereoisomer, atropisomer, tautomer, or pharmaceutically acceptable salt thereof to be administered will be governed by such considerations, and is the minimum amount necessary to ameliorate, or treat the hyperproliferative disorder.

As a general proposition, the initial pharmaceutically effective amount of the compound or a stereoisomer, atropisomer, tautomer, or pharmaceutically acceptable salt thereof administered parenterally per dose will be in the range of about 0.01-100 mg/kg, namely about 0.1 to 20 mg/kg of patient body weight per day, with the typical initial range of compound used being 0.3 to 15 mg/kg/day. In another embodiment, a pharmaceutical composition described herein comprises an effective amount of a compound or a stereoisomer, atropisomer, tautomer, or pharmaceutically acceptable salt thereof described herein in an amount of about: 1 mg-10 mg; 10 mg-25 mg; 20 mg-50 mg; 50 mg-75 mg; 70 mg-100 mg; 100 mg-150 mg; 100 mg-200 mg; 100 mg-500 mg; 200 mg-500 mg; 250 mg-500 mg; 500 mg-1000 mg; or 750 mg-1000 mg.

Acceptable pharmaceutically acceptable excipients are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). The active pharmaceutical ingredients can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations of compounds or pharmaceutically acceptable salts thereof as described herein may be prepared. Suitable examples of sustained-release preparations include semipermeable matrices of solid hydrophobic polymers containing a compound or pharmaceutically acceptable salt thereof as described herein, which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate) and poly-D-(−)-3-hydroxybutyric acid.

The formulations include those suitable for the administration routes detailed herein. The formulations can conveniently be presented in unit dosage form and can be prepared by any methods. Techniques and formulations generally are found in Remington's Pharmaceutical Sciences (Mack Publishing Co., Easton, PA). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of a compound or a stereoisomer, atropisomer, tautomer, or pharmaceutically acceptable salt thereof as described herein suitable for oral administration can be prepared as discrete units such as pills, capsules, cachets or tablets each containing a predetermined amount of such compound or a stereoisomer, atropisomer, tautomer, or pharmaceutically acceptable salt thereof. Compressed tablets can be prepared by compressing in a suitable machine the active ingredient in a free-flowing form such as a powder or granules, optionally mixed with a binder, lubricant, inert diluent, preservative, surface active or dispersing agent. Molded tablets can be made by molding in a suitable machine a mixture of the powdered active ingredient moistened with an inert liquid diluent. The tablets can optionally be coated or scored and optionally are formulated so as to provide slow or controlled release of the active ingredient therefrom. Tablets, troches, lozenges, aqueous or oil suspensions, dispersible powders or granules, emulsions, hard or soft capsules, e.g., gelatin capsules, syrups or elixirs can be prepared for oral use. Formulations of compounds or pharmaceutically acceptable salts thereof as described herein intended for oral use can be prepared according to any method for the manufacture of pharmaceutical compositions and such compositions can contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients can be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets can be uncoated or can be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax can be employed.

For treatment of the eye or other external tissues, e.g., mouth and skin, the formulations are preferably applied as a topical ointment or cream containing the active ingredient(s) in an amount of, for example, 0.075 to 20% w/w. When formulated in an ointment, the active ingredients can be employed with either a paraffinic or a water-miscible ointment base. Alternatively, the active ingredients can be formulated in a cream with an oil-in-water cream base. If desired, the aqueous phase of the cream base can include a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations can desirably include a compound which enhances absorption or penetration of the active ingredient through the skin or other affected areas. Examples of such dermal penetration enhancers include dimethyl sulfoxide and related analogs. The oily phase of the emulsions of compositions provided herein can be constituted from known ingredients in a known manner. While the phase can comprise merely an emulsifier, it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulsifiers and emulsion stabilizers suitable for use in the formulation of described herein include Tween® 60, Span® 80, cetostearyl alcohol, benzyl alcohol, myristyl alcohol, glyceryl mono-stearate and sodium lauryl sulfate.

Aqueous suspensions comprising a compound or a stereoisomer, atropisomer, tautomer, or pharmaceutically acceptable salt thereof as described herein can contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension can also contain one or more preservatives such as ethyl or n-propyl p-hydroxybenzoate, one or more coloring agents, one or more flavoring agents and one or more sweetening agents, such as sucrose or saccharin.

The pharmaceutical compositions of a compound or a stereoisomer, atropisomer, tautomer, or pharmaceutically acceptable salt thereof as described herein can be in the form of a sterile injectable preparation, such as a sterile injectable aqueous or oleaginous suspension. This suspension can be formulated using suitable dispersing or wetting agents and suspending agents which have been mentioned above. The sterile injectable preparation can also be a sterile injectable solution or suspension in a non-toxic parenterally acceptable diluent or solvent, such as a solution in 1,3-butanediol or prepared as a lyophilized powder. Among the acceptable vehicles and solvents that can be employed are water, Ringer's solution and isotonic sodium chloride solution. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables.

The amount of active ingredient that can be combined with the carrier material to produce a single dosage form will vary depending upon the host treated and the particular mode of administration. For example, a time-release formulation intended for oral administration to humans can contain approximately 1 to 1000 mg of active material compounded with an appropriate and convenient amount of carrier material which can vary from about 5 to about 95% of the total compositions (weight:weight). The pharmaceutical composition can be prepared to provide easily measurable amounts for administration. For example, an aqueous solution intended for intravenous infusion can contain from about 3 to 500 μg of the active ingredient per milliliter of solution in order that infusion of a suitable volume at a rate of about 30 mL/hr can occur.

Formulations suitable for parenteral administration include aqueous and non-aqueous sterile injection solutions which can contain anti-oxidants, buffers, bacteriostats and solutes which render the formulation isotonic with the blood of the intended recipient; and aqueous and non-aqueous sterile suspensions which can include suspending agents and thickening agents.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of about 0.5 to 20% w/w, for example about 0.5 to 10% w/w, for example about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration can be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration can be prepared according to conventional methods and can be delivered with other therapeutic agents such as compounds heretofore used in the treatment or prophylaxis disorders as described below.

Formulations suitable for vaginal administration can be presented as pessaries, tampons, creams, gels, pastes, foams or spray formulations containing in addition to the active ingredient such carriers considered to be appropriate.

The formulations can be packaged in unit-dose or multi-dose containers, for example sealed ampoules and vials, and can be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

In one embodiment, the compound or a stereoisomer, atropisomer, tautomer, or pharmaceutically acceptable salt thereof are formulated as a prodrug. The term prodrug as used herein refers to a derivative of a compound that can be hydrolyzed, oxidized, or cleaved under biological conditions to provide the compound or a stereoisomer, atropisomer, tautomer, or pharmaceutically acceptable salt thereof. A prodrug as defined herein includes derivatives comprising one or more moieties that modulate or improve one or more physical, physiological or pharmaceutical property such as, but not limited to, solubiliy, permeability, uptake, biodistribution, metabolic stability, onset of action or some other druglike property, and is transformed to the bioactive or more biologically active substance as provided herein. In one embodiment, a prodrug herein has no biological activity until release of the compound or pharmaceutically acceptable salt thereof.

### Methods of Administration

Compounds or a stereoisomer, atropisomer, tautomer, or pharmaceutically acceptable salt thereof described herein can be administered by any route appropriate to the condition to be treated. Suitable routes include oral, parenteral (including subcutaneous, intramuscular, intravenous (IV), intraarterial, intradermal, intrathecal and epidural), transdermal, rectal, nasal, topical (including buccal and sublingual), vaginal, intraperitoneal, intrapulmonary and intranasal. In one embodiment, a compound or a stereoisomer, atropisomer, tautomer, or pharmaceutically acceptable salt thereof described herein is administered orally or by IV. For local immunosuppressive treatment, the compounds can be administered by intralesional administration, including perfusing or otherwise contacting the graft with the inhibitor before transplantation. It will be appreciated that the preferred route can vary with for example the condition of the recipient. Where the compound or a stereoisomer, atropisomer, tautomer, or pharmaceutically acceptable salt thereof is administered orally, it can be formulated as a pill, capsule, tablet, etc. with a pharmaceutically acceptable carrier or excipient. Where the compound or a stereoisomer, atropisomer, tautomer, or pharmaceutically acceptable salt thereof is administered parenterally, it can be formulated with a pharmaceutically acceptable parenteral vehicle and in a unit dosage injectable form, as detailed below.

Thus, in one aspect provided herein is a pharmaceutical composition comprising a compound or pharmaceutically acceptable salt thereof as described herein and one or more pharmaceutically acceptable excipients. In one embodiment, compounds or a stereoisomer, atropisomer, tautomer, or pharmaceutically acceptable salt thereof described herein are administered as pharmaceutical compositions capable of being administered to a subject orally or parenterally. The compounds or a stereoisomer, atropisomer, tautomer, or pharmaceutically acceptable salt thereof described herein can be formulated for topical or parenteral use where the compound or a stereoisomer, atropisomer, tautomer, or pharmaceutically acceptable salt thereof is dissolved or otherwise suspended in a solution suitable for injections, suspensions, syrups, creams, ointments, gels, sprays, solutions and emulsions.

Oral administration can promote patient compliance in taking the compound (e.g. formulated as a pharmaceutical composition), thereby increasing compliance and efficacy. Oral pharmaceutical compositions comprising a compound described herein include, but are not limited to, tablets (e.g. coated, non-coated and chewable) and capsules (e.g. hard gelatin capsules, soft gelatin capsules, enteric coated capsules, and sustained release capsules). Tablets can be prepared by direct compression, by wet granulation, or by dry granulation. Oral pharmaceutical compositions comprising a compound described herein can be formulated for delayed or prolonged release.

A dose to treat human patients can range from about 10 mg to about 1000 mg of a compound described herein. A typical dose can be about 100 mg to about 300 mg of the compound. A dose can be administered once a day (QID), twice per day (BID), or more frequently, depending on the pharmacokinetic and pharmacodynamic properties, including absorption, distribution, metabolism, and excretion of the particular compound. Administration as used herein refers to the frequency of dosing and not, for example, the number of individual units a patient described herein must take for a dose. Thus, in some embodiments, a patient may take two or more dosage units (e.g. two or more pills/tablets/capsules) QD. In addition, toxicity factors can influence the dosage and administration regimen. When administered orally, the pill, capsule, or tablet can be ingested daily or less frequently for a specified period of time. The regimen can be repeated for a number of cycles of therapy.

### Methods of Treating and Uses

The compounds or a stereoisomer, atropisomer, tautomer, or pharmaceutically acceptable salt thereof described herein are useful as Ras inhibitors. In one aspect, the compounds or a stereoisomer, atropisomer, tautomer, or pharmaceutically acceptable salt thereof described herein are useful as KRas inhibitors. In another aspect, the compound or a stereoisomer, atropisomer, tautomer, or pharmaceutically acceptable salt thereof described herein are useful as NRas inhibitors. In another aspect, the compound or a stereoisomer, atropisomer, tautomer, or pharmaceutically acceptable salt thereof described herein are useful as HRas inhibitors. In one embodiment, the compounds or a stereoisomer, atropisomer, tautomer, or pharmaceutically acceptable salt thereof described herein are useful as G12D Ras inhibitors, and as G12D KRas inhibitors.

Provided herein are methods of contacting a cell, such as an ex vivo cell, with a compound or a stereoisomer, atropisomer, tautomer, or pharmaceutically acceptable salt thereof described herein, to inhibit Ras activity (e.g., KRas activity) in the cell. In another embodiment, the activity is mutant G12D KRas activity.

Further provided herein are methods of treating a cancer comprising a KRas mutation, the method comprising administering to a patient having such cancer, an effective amount of a compound or a stereoisomer, atropisomer, tautomer, or pharmaceutically acceptable salt thereof or a pharmaceutical composition as described herein. In one embodiment, the KRas mutation is a KRas^{G12D} mutation.

In one embodiment, the methods further comprise testing a sample (e.g. as set forth herein) from the patient before administration of a compound of pharmaceutically acceptable salt thereof described herein for the absence or presence of a KRas^{G12D} mutation. In one such embodiment, a compound or stereoisomer, atropisomer, tautomer, or pharmaceutically acceptable salt thereof or pharmaceutical composition described herein is administered to the patient after the patient sample is determined to be positive for (e.g. the presence of) a KRas^{G12D} mutation.

The methods of treating a cancer described herein relate to the treatment of cancer such as acute myeloid leukemia, cancer in adolescents, childhood adrenocortical carcinoma, AIDS-related cancers (e.g. lymphoma and Kaposi's sarcoma), anal cancer, appendix cancer, astrocytomas, atypical teratoid rhabdoid tumor, basal cell carcinoma, bile duct cancer, bladder cancer, bone cancer, brain stem glioma, brain tumor, breast cancer, bronchial tumors, Burkitt lymphoma, carcinoid tumor, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, Ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin's lymphoma, hypopharyngeal cancer, intraocular melanoma, islet cell tumors, pancreatic neuroendocrine tumors, kidney cancer, laryngeal cancer, lip and oral cavity cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, Merkel cell carcinoma, malignant mesothelioma, malignant fibrous histiocytoma of bone and osteosarcoma, nasal cavity and paranasal sinus cancer, nasopharyngeal cancer, neuroblastoma, non-Hodgkin's lymphoma, non-small cell lung cancer (NSCLC), oral cancer, oropharyngeal cancer, ovarian cancer, pancreatic cancer, papillomatosis, paraganglioma, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or viral-induced cancer.

In some embodiments, the cancer is a hematological cancer, pancreatic cancer, MYH associated polyposis, colorectal cancer or lung cancer. In one embodiment, the cancer is lung cancer, colorectal cancer, appendicial cancer, or pancreatic cancer. In one embodiment, the cancer is pancreatic cancer, lung cancer, or colon cancer. The lung cancer can be adenocarcinoma, non-small cell lung cancer (NSCLC), or small cell lung cancer (SCLC). In one embodiment, the cancer is colorectal cancer. In another embodiment, the cancer is pancreatic cancer. In one embodiment, the cancer is lung adenocarcinoma.

The methods provided herein can also comprise testing a sample from the patient before administration of a compound or stereoisomer, atropisomer, tautomer, or pharmaceutically acceptable salt thereof described herein for the absence or presence of a KRas^{G12D} mutation. In one embodiment, a compound, stereoisomer, atropisomer, tautomer, or pharmaceutically acceptable salt thereof or pharmaceutical composition is administered to the patient after the patient sample shows the presence of a KRas^{G12D} mutation. In one embodiment, a compound or stereoisomer, atropisomer, tautomer, or pharmaceutically acceptable salt thereof described herein is not administered unless a patient sample comprises a KRas^{G12D} mutation.

In one embodiment, the cancer is pancreatic cancer, lung cancer, or colorectal cancer. In another embodiment, the cancer is tissue agnostic (comprises a KRas^{G12D} mutation).

Further provided herein are methods of treating lung cancer comprising a KRas^{G12D} mutation in a patient having such a lung cancer, the method comprising administering to the patient an effective amount of a compound or a stereoisomer, atropisomer, tautomer, or pharmaceutically acceptable salt thereof (or a pharmaceutical composition comprising the same) described herein to the patient. In one embodiment, the lung cancer is non-small cell lung carcinoma (NSCLC). The NSCLC can be, for example, adenocarcinoma, squamous-cell lung carcinoma or large-cell lung carcinoma. In another embodiment, the lung cancer is small cell lung carcinoma. In still another embodiment, the lung cancer is glandular tumors, carcinoid tumors or undifferentiated carcinomas. The lung cancer can be stage I or II lung cancer. In one embodiment, the lung cancer is stage III or IV lung cancer. The methods provided herein include administration of the compound as a 1 L therapy.

Still further provided herein are methods of treating pancreatic cancer comprising a KRas^{G12D} mutation in a patient having such pancreatic cancer, the method comprising administering to the patient an effective amount of a compound or stereoisomer, atropisomer, tautomer, or pharmaceutically acceptable salt thereof described herein to the patient. In one embodiment, the patient has been previously treated with radiation and one or more chemotherapy agents. In one embodiment, the pancreatic cancer is stage 0, I, or II. In another embodiment, the pancreatic cancer is stage III or stage IV.

Still further provided herein are methods of treating colon cancer comprising a KRas^{G12D} mutation in a patient having such colon cancer, the method comprising administering a to the patient an effective amount of a compound or stereoisomer, atropisomer, tautomer, or pharmaceutically acceptable salt thereof described herein to the patient. In one embodiment, the colon cancer is stage I or II. In another embodiment, the colon cancer is stage III or stage IV.

Further provided herein are methods of treating tissue agnostic cancer comprising a KRas^{G12D} mutation. In one embodiment of such methods, the method comprises:
(a) determining the absence or presence of a KRas^{G12D} mutation in a sample taken from a patient with a suspected diagnosed cancer; and
(b) administering to the patient an effective amount of a compound or stereoisomer, atropisomer, tautomer, or pharmaceutically acceptable salt thereof described herein.

In one embodiment of such methods, the patient is diagnosed with a cancer described herein. In another embodiment of such methods, the sample is a tumor sample taken from the subject. In one such embodiment, the sample is taken before administration of any therapy. In another such embodiment, the sample is taken before administration of a compound or stereoisomer, atropisomer, tautomer, or pharmaceutically acceptable salt thereof described herein and after administration of another chemotherapeutic agent. In another embodiment of such methods, the compound or stereoisomer, atropisomer, tautomer, or pharmaceutically acceptable salt thereof described herein is administered as provided herein (e.g. orally or IV).

Also provided herein is a compound or stereoisomer, atropisomer, tautomer, or pharmaceutically acceptable salt thereof for use as a therapeutically active substance. In one such embodiment, the compound or stereoisomer, atropisomer, tautomer, or pharmaceutically acceptable salt thereof can be for the therapeutic treatment of a cancer comprising a KRas^{G12D} mutation. Further provided herein is a compound or stereoisomer, atropisomer, tautomer, or pharmaceutically acceptable salt thereof for the therapeutic and/or prophylactic treatment of a cancer comprising a KRas^{G12D} mutation. In one embodiment, the compound or stereoisomer, atropisomer, tautomer, or pharmaceutically acceptable salt thereof is used in the preparation of a medicament for the therapeutic treatment of a cancer comprising a KRas^{G12D} mutation. Still further provided herein are uses of a compound or stereoisomer, atropisomer, tautomer, or pharmaceutically acceptable salt thereof as described herein in the manufacture of a medicament for inhibiting tumor metastasis.

Further provided herein are methods for inhibiting tumor metastasis, the method comprising administering to a patient having a tumor a therapeutically effective amount of a compound or stereoisomer, atropisomer, tautomer, or pharmaceutically acceptable salt thereof described herein. In one embodiment, the inhibition is of a tumor comprising a KRas^{G12D} mutation. In another embodiment, inhibiting tumor metastasis in a patient described herein results in reduction of tumor size. In another embodiment, inhibiting tumor metastasis in a patient described herein results in stabilizing (e.g. no further growth) of tumor size. In another embodiment, inhibiting tumor metastasis in a patient described herein results in remission of the cancer and/or its symptoms.

Further provided herein are methods for inhibiting proliferation of a cell population, the method comprising contacting the cell population with a compound or stereoisomer, atropisomer, tautomer, or pharmaceutically acceptable salt thereof described herein. In one embodiment, the cell population is in a human patient. In another embodiment, the cell population comprises a KRas^{G12D} mutation.

Further provided herein are methods of inhibiting KRas in a patient in need of therapy, comprising administering to the patient a therapeutically effective amount of a compound or stereoisomer, atropisomer, tautomer, or pharmaceutically acceptable salt thereof described herein. In one embodiment, the KRas inhibited is KRas^{G12D}. In another embodiment, inhibiting KRas results in decreased tumor size. In another embodiment, inhibiting KRas results in remission of the cancer and/or its symptoms.

Further provided herein are methods for regulating activity of a KRas mutant protein, the method comprising reacting the mutant protein with a compound or stereoisomer, atropisomer, tautomer, or pharmaceutically acceptable salt thereof described herein. In one embodiment, the mutant protein comprises a KRas^{G12D} mutation. In one embodiment, the activity of KRas is decreased after contacting with a compound or stereoisomer, atropisomer, tautomer, or pharmaceutically acceptable salt thereof described herein. In another embodiment, the downregulation of activity of the KRas mutant protein treats a cancer described herein in a patient described herein. In another embodiment, the downregulation of activity of the KRas mutant protein results in decreased tumor size. In another embodiment, the downregulation of activity of the KRas mutant protein results in remission of a cancer described herein and/or its symptoms.

In some embodiments, the methods provided herein comprise inhibiting KRas^{G12D} activity in a cell by contacting said cell with an amount of a compound or stereoisomer, atropisomer, tautomer, or pharmaceutically acceptable salt thereof described herein sufficient to inhibit the activity of KRas^{G12D} in said cell. In some embodiments, the methods provided herein comprise inhibiting KRas^{G12D} activity in a tissue by contacting said tissue with an amount of a compound or stereoisomer, atropisomer, tautomer, or pharmaceutically acceptable salt thereof described herein sufficient to inhibit the activity of KRas^{G12D} in said tissue. In some embodiments, the methods provided herein comprise inhibiting KRas^{G12D} activity in a patient described herein by contacting said patient with an amount of a compound or stereoisomer, atropisomer, tautomer, or pharmaceutically acceptable salt thereof described herein sufficient to inhibit the activity of KRas^{G12D} in said patient.

Further provided herein are methods for preparing a labeled KRas^{G12D} mutant protein, the method comprising reacting a KRas^{G12D} mutant protein with a labeled compound or stereoisomer, atropisomer, tautomer, or pharmaceutically acceptable salt thereof described herein to result in the labeled KRas^{G12D} mutant protein. In one embodiment, the label is an imaging agent. In one embodiment, the labeled KRas^{G12D} can be used to detect the absence or presence of G12D mutant KRas in a patient sample, thereby detecting the presence or absence of a cancer mediated by mutant KRas.

Still further provided herein are methods of inhibiting Ras-mediated cell signaling. In one embodiment, the methods comprise contacting a cell with an effective amount of one or more compounds or a stereoisomer, atropisomer, tautomer, or pharmaceutically acceptable salt thereof disclosed herein thereof. Inhibition of Ras-mediated signal transduction can be assessed and demonstrated by a wide variety of ways known in the art. Non-limiting examples include a showing of (a) a decrease in GTPase activity of Ras; (b) a decrease in GTP binding affinity or an increase in GDP binding affinity; (c) an increase in K off of GTP or a decrease in K off of GDP; (d) a decrease in the levels of signaling transduction molecules downstream in the Ras pathway, such as a decrease in pMEK level; and/or (e) a decrease in binding of Ras complex to downstream signaling molecules including but not limited to Raf. Kits and commercially available assays can be utilized for determining one or more of the above.

KRas mutations, including G12D mutants, have also been identified in hematological malignancies (e.g., cancers that affect blood, bone marrow, and/or lymph nodes). Accordingly, certain embodiments are directed to administration of a disclosed compound or stereoisomer, atropisomer, tautomer, or pharmaceutically acceptable salt thereof (e.g., in the form of a pharmaceutical composition) as described herein to a patient in need of treatment of a hematological malignancy. Such malignancies include, but are not limited to leukemias and lymphomas. For example, the presently disclosed compounds can be used for treatment of diseases such as acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMoL) and/or other leukemias. In other embodiments, the compounds or a pharmaceutically acceptable salt thereof described herein are useful for treatment of lymphomas such as all subtypes of Hodgkin's lymphoma or non-Hodgkin's lymphoma.

Determining whether a tumor or cancer comprises a KRas^{G12D} mutation can be undertaken by assessing the nucleotide sequence encoding the KRas protein, by assessing the amino acid sequence of the KRas protein, or by assessing the characteristics of a putative KRas mutant protein. The sequence of wild-type human KRas (e.g. Accession No. NP203524) is known in the art.

Methods for detecting a mutation in a KRas nucleotide sequence are known by those of skill in the art. These methods include, but are not limited to, polymerase chain reaction-restriction fragment length polymorphism (PCR-RFLP) assays, polymerase chain reaction-single strand conformation polymorphism (PCR—SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples are evaluated for G12D KRas mutations by real-time PCR. In real-time PCR, fluorescent probes specific for the KRas G12D mutation are used. When a mutation is present, the probe binds and fluorescence is detected. In some embodiments, the KRas G12D mutation is identified using a direct sequencing method of specific regions (e.g., exon 2 and/or exon 3) in the KRas gene. This technique will identify all possible mutations in the region sequenced.

Methods for determining whether a tumor or cancer comprises a KRas^{G12D} mutation can use a variety of samples. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is processed to a cell lysate. In some embodiments, the sample is processed to DNA or RNA.

Further provided herein are uses of a compound or stereoisomer, atropisomer, tautomer, or pharmaceutically acceptable salt thereof described herein, in the manufacture of a medicament for treating cancer. In some embodiments, the medicament is formulated for oral administration. In some embodiments, the medicament is formulated for injection (e.g. IV administration). In some embodiments, the cancer is comprises a KRas^{G12D} mutation. In some embodiments, the cancer is a hematological cancer, pancreatic cancer, MYH associated polyposis, colorectal cancer or lung cancer. In one embodiment, the cancer is lung cancer, colorectal cancer, or pancreatic cancer. In one embodiment, the cancer is colorectal cancer. In another embodiment, the cancer is pancreatic cancer. In some embodiments, the cancer is lung adenocarcinoma. In some embodiments, are uses of a compound or stereoisomer, atropisomer, tautomer, or pharmaceutically acceptable salt thereof described herein, in the manufacture of a medicament for inhibiting tumor metastasis.

Further provided herein is a compound or a pharmaceutically acceptable salt thereof described herein, for use in a method of treating cancer. In one embodiment, the cancer comprises a KRas^{G12D} mutation. In one such embodiment, the cancer is a hematological cancer, pancreatic cancer, MYH associated polyposis, colorectal cancer or lung cancer. In one such embodiment, the cancer is lung cancer, colorectal cancer, or pancreatic cancer. In one such embodiment, the cancer is colorectal cancer. In one such embodiment, the cancer is pancreatic cancer. In one such embodiment, the cancer is lung adenocarcinoma.

### Combination Therapies

The compounds or a stereoisomer, atropisomer, tautomer, or pharmaceutically acceptable salt thereof described herein may be employed alone or in combination with other therapeutic agents for the treatment of a disease or disorder described herein. The second compound of the pharmaceutical combination formulation or dosing regimen preferably has complementary activities to the compound or a pharmaceutically acceptable salt thereof described herein such that they do not adversely affect each other. The combination therapy may provide “synergy” and prove “synergistic”, i.e., the effect achieved when the active ingredients used together is greater than the sum of the effects that results from using the compounds separately.

The combination therapy may be administered as a simultaneous or sequential regimen. When administered sequentially, the combination may be administered in two or more administrations. The combined administration includes co-administration, using separate formulations or a single pharmaceutical formulation, and consecutive administration in either order, wherein preferably there is a time period while both (or all) active agents simultaneously exert their biological activities.

Combination therapies herein comprise the administration of a compound or stereoisomer, atropisomer, tautomer, or pharmaceutically acceptable salt thereof described herein, and the use of at least one other treatment method. The amounts of the compound or stereoisomer, atropisomer, tautomer, or pharmaceutically acceptable salt thereof described herein and the other pharmaceutically active agent(s) and the relative timings of administration will be selected in order to achieve the desired combined therapeutic effect.

In various embodiments of the method, the additional therapeutic agent is an epidermal growth factor receptor (EGFR) inhibitor, phosphatidylinositol kinase (PI3K) inhibitor, insulin-like growth factor receptor (IGF1R) inhibitor, a Janus kinase (JAK) inhibitor, a Met kinase inhibitor, a SRC family kinase inhibitor, a mitogen-activated protein kinase (MEK) inhibitor, an extracellular-signal-regulated kinase (ERK) inhibitor, a topoisomerase inhibitor (such as irinotecan, or such as etoposide, or such as doxorubicin), a taxane (such as anti-microtubule agents including paclitaxel and docetaxel), an anti-metabolite agent (such as 5-FU or such as gemcitabine), or an alkylating agent (such as cisplatin or such as cyclophosphamide), or a taxane.

In some embodiments, the additional therapeutic agent is an epidermal growth factor receptor (EGFR) inhibitor, such as Erlotinib or such as Afatinib. In some embodiments the additional therapeutic agent is gefitinib, osimertinib, or dacomitinib. In some embodiments the additional therapeutic agent is a monoclonal antibody such as cetuximab (Erbitux) or panitumumab (Vectibix). In some embodiments the GFR inhibitor is a dual or pan- HER inhibitor. In other embodiments, the additional therapeutic agent is a phosphatidylinositol-3-kinase (PI3K) inhibitor, such as GDC-0077, GDC-0941, MLN1117, BYL719 (Alpelisib) or BKM120 (Buparlisib). GDC-0941 refers to 2-(1H-indazol-4-yl)-6-(4-methanesulfonyl-piperazin-1-ylmethyl)-4-morpholin-4-yl-thieno[3,2-d]pyrimidine or a salt thereof (e.g., bismesylate salt).

In still other embodiments, the additional therapeutic agent is an insulin-like growth factor receptor (IGF1R) inhibitor. For example, in some embodiments the insulin-like growth factor receptor (IGF1R) inhibitor is NVP-AEW541. In other embodiments, the additional therapeutic agent is IGOSI-906 (Linsitinib), BMS-754807, or in other embodiments the additional therapeutic agent is a neutralizing monoclonal antibody specific to IGF1R such as AMG-479 (ganitumab), CP-751,871 (figitumumab), IMC-A12 (cixutumumab), MK-0646 (dalotuzumab), or R-1507 (robatumumab).

In some other embodiments, the additional therapeutic agent is a Janus kinase (JAK) inhibitor. In some embodiments, the additional therapeutic agent is CYT387, GLPG0634, Baricitinib, Lestaurtinib, momelotinib, Pacritinib, Ruxolitinib, or TG101348.

In some other embodiments, the additional therapeutic agent is an anti-glypican 3 antibody. In some embodiments, the anti-glypican 3 antibody is codrituzumab.

In some other embodiments, the additional therapeutic agent is an antibody drug conjugate (ADC). In some embodiments, the ADC is polatuzumab vedotin, RG7986, RG7882, RG6109, or RO7172369.

In some other embodiments, the additional therapeutic agent is an MDM2 antagonist. In some embodiments, the MDM2 antagonist is idasanutlin.

In some other embodiments, the additional therapeutic agent is an agonistic antibody against CD40. In some embodiments, the agonistic antibody against CD40 is selicrelumab (RG7876).

In some other embodiments, the additional therapeutic agent is a bispecific antibody. In some embodiments, the bispecific antibody is RG7828 (BTCT4465A), RG7802, RG7386 (FAP-DR5), RG6160, RG6026, ERY974, or anti-HER2/CD3.

In some other embodiments, the additional therapeutic agent is a targeted immunocytokine. In some embodiments, the targeted immunocytokine is RG7813 or RG7461.

In some other embodiments, the additional therapeutic agent is an antibody targeting colony stimulating factor-1 receptor (CSF-1R). In some embodiments, the CSF-1R antibody is emactuzumab.

In some other embodiments, the additional therapeutic agent is a personalised cancer vaccine. In some embodiments, the personalised cancer vaccine is RG6180.

In some other embodiments, the additional therapeutic agent is an inhibitor of BET (bromodomain and extraterminal family) proteins (BRD2/3/4/T). In some embodiments, the BET inhibitor is RG6146.

In some other embodiments, the additional therapeutic agent is an antibody designed to bind to TIGIT. In some embodiments, the anti-TIGIT antibody is RG6058 (MTIG7192A).

In some other embodiments, the additional therapeutic agent is a selective estrogen receptor degrader (SERD). In some other embodiments, the SERD is RG6047 (GDC-0927) or RG6171 (GDC-9545, giredestrant).

In some other embodiments the additional therapeutic agent is an MET kinase inhibitor, such as Crizotinib, tivantinib, AMG337, cabozantinib, or foretinib. In other embodiments the additional therapeutic agent is a neutralizing monoclonal antibody to MET such as onartuzumab.

In more embodiments, the additional therapeutic agent is a SRC family non-receptor tyrosine kinase inhibitor. For example, in some embodiments the additional therapeutic agent is an inhibitor of the subfamily of SRC family non-receptor tyrosine kinases. Exemplary inhibitors in this respect include Dasatinib. Other examples in this regard include Ponatinib, saracatinib, and bosutinib.

In yet other embodiments, the additional therapeutic agent is a mitogen-activated protein kinase (MEK) inhibitor. In some of these embodiments, the mitogen-activated protein kinase (MEK) inhibitor is trametinib, selumetinib, COTELLIC® (cobimetinib), PD0325901, or RO5126766. In other embodiments the MEK inhibitor is GSK-1120212, also known as trametinib.

In yet other embodiments, the additional therapeutic agent is an extracellular-signal-regulated kinase (ERK) inhibitor. In some of these embodiments, the mitogen-activated protein kinase (MEK) inhibitor is SCH722984 or GDC-0994.

In other embodiments the protein kinase inhibitor is taselisib, ipatasertib, GDC-0575, GDC-5573 (HM95573), RG6114 (GDC-0077), CKI27, Afatinib, Axitinib, Atezolizumab, Bevacizumab, Bostutinib, Cetuximab, Crizotinib, Dasatinib, Erlotinib, Fostamatinib, Gefitinib, Imatinib, Lapatinib, Lenvatinib, Ibrutinib, Nilotinib, Panitumumab, Pazopanib, Pegaptanib, Ranibizumab, Ruxolitinib, Sorafenib, Sunitinib, SU6656, Trastuzumab, Tofacitinib, Vandetanib, or Vemurafenib. In still more embodiments, the additional therapeutic agent is a topoisomerase inhibitor. In some of these embodiments, the topoisomerase inhibitor is Irinotecan. In some more embodiments, the additional therapeutic agent is a taxane. Exemplary taxanes include Taxol and Docetaxel.

In addition to the above additional therapeutic agent, other chemotherapeutics are presently known in the art and can be used in combination with the compounds and pharmaceutically acceptable salts thereof described herein. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

Non-limiting examples are chemotherapeutic agents, cytotoxic agents, and non-peptide small molecules such as Gleevec® (Imatinib Mesylate), Velcade® (bortezomib), Casodex (bicalutamide), Iressa® (gefitinib), and Adriamycin as well as a host of chemotherapeutic agents. Non-limiting examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methyl melamines including altretamine, triethylenemelamine, triethylenephosphoramide, triethylenethiophosphaoramide and trimethylol melamine; nitrogen mustards such as chlorambucil, chlornaphazine, cyclophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™ chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; polysaccharide K; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins; capecitabine; and pharmaceutically acceptable salts, acids or derivatives of any of the above. Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4 (5)-imidazoles, 4-hydroxytamoxifen, trioxifene, keoxifene, LY 117018, onapristone, and toremifene (Fareston); anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; Xeloda®; ibandronate; camptothecin-11 (CPT-11); topoisomerase inhibitor RFS 2000; and difluoromethylornithine (DMFO). Where desired, the compounds or pharmaceutical acceptable salts thereof or pharmaceutical composition as described herein can be used in combination with commonly prescribed anti-cancer drugs such as Herceptin®, Avastin®, Gazyva®, Tecentriq®, Alecensa®, Perjeta®, Venclexta™, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126 or Zosuquidar.

The exact method for administering the compound and the additional therapeutic agent will be apparent to one of ordinary skill in the art. In some exemplary embodiments the compound and the additional therapeutic agent are co-administered. In other embodiments, the compound and the additional therapeutic agent are separately administered.

In some embodiments, the compound and the additional therapeutic agent are administered with the second agent simultaneously or separately. This administration in combination can include simultaneous administration of the two agents in the same dosage form, simultaneous administration in separate dosage forms, and separate administration. That is, the compound and any of the additional therapeutic agents described herein can be formulated together in the same dosage form and administered simultaneously. Alternatively, the compound and any of the additional therapeutic agents described herein can be simultaneously administered, wherein both the agents are present in separate formulations. In another alternative, the compound can be administered just followed by any of the additional therapeutic agents described herein, or vice versa. In some embodiments of the separate administration protocol, the compound and any of the additional therapeutic agents described herein are administered a few minutes apart, or a few hours apart, or a few days apart.

### Articles of Manufacture

Also provided herein are articles of manufacture, or “kit”, containing materials useful for the treatment of a cancer provided herein. In one embodiment, the kit comprises a container comprising compound or stereoisomer, atropisomer, tautomer, or pharmaceutically acceptable salt thereof described herein. The kit may further comprise a label or package insert on or associated with the container. Suitable containers include, for example, bottles, vials, syringes, blister pack, etc. The container may be formed from a variety of materials such as glass or plastic. The container may hold a compound or a pharmaceutically acceptable salt thereof described herein or a formulation thereof which is effective for treating the condition and may have a sterile access port (for example, the container may be an intravenous solution bag or a vial having a stopper pierceable by a hypodermic injection needle). At least one active agent in the composition is a compound or a pharmaceutically acceptable salt thereof described herein. Alternatively, or additionally, the article of manufacture may further comprise a second container comprising a pharmaceutical diluent, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

In another embodiment, the kits are suitable for the delivery of solid oral forms of a compound or a pharmaceutically acceptable salt thereof described herein, such as tablets or capsules. Such a kit can include a number of unit dosages. An example of such a kit is a “blister pack”. Blister packs are well known in the packaging industry and are widely used for packaging pharmaceutical unit dosage forms.

### EMBODIMENTS

Provided below are some exemplary embodiments of the invention described herein.

Embodiment 1. A compound having formula (I):

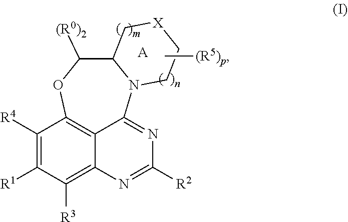

or a stereoisomer, atropisomer, tautomer, or pharmaceutically acceptable salt thereof,
wherein;
X is O or NR⁶;
n is 1, 2, or 3;
m is 1, 2, or 3;
p is 0, 1, or 2;
wherein n and m together make a 6-, 7-, or 8-membered ring A;
each R⁰ is independently hydrogen or methyl;
R¹ is R⁷-substituted or unsubstituted napthyl, R⁷-substituted or unsubstituted isoquinolinyl, R⁷-substituted or unsubstituted indazolyl, R⁷-substituted or unsubstituted benzothiazolyl, R^{7A}-substituted or unsubstituted phenyl, or R^{7A}-substituted or unsubstituted pyridinyl;
each R⁷ is independently hydrogen, halogen, —OH, NH₂, N(Me)₂, unsubstituted C₁₋₃ alkyl, unsubstituted C₁₋₃ haloalkyl, or unsubstituted cyclopropyl;
each R^{7A} is independently hydrogen, halogen, NH₂, N(Me)₂, unsubstituted C₁₋₃ alkyl, unsubstituted C₁₋₃ haloalkyl, or unsubstituted cyclopropyl;
R² is hydrogen, L¹-O-L²-R⁸, R^{8A}-substituted or unsubstituted C₁₋₃ alkyl, or R^{8B}-substituted or unsubstituted 4-10 membered heterocycle;
wherein when R² is hydrogen, R¹ is R⁷-substituted indazolyl, and n and m are 1, then
p is not zero and R⁶ is not H;
L¹ is a bond or R^{L1}-substituted or unsubstituted C₁₋₃ alkylene;
R^{L1} is halogen or unsubstituted C₁₋₃ alkyl;
L² is a bond or unsubstituted C₁₋₃ alkylene;
R⁸ is R⁹-substituted or unsubstituted 4-10 membered heterocycle comprising N, S, or O;
each R⁹ is independently halogen, oxo, —OCF₃, —OCHF₂, —OCH₂F, unsubstituted C₁₋₃ alkyl, unsubstituted C₁₋₃ haloalkyl, unsubstituted C₁₋₃ alkoxy, R¹⁰-substituted or unsubstituted C₁₋₃ alkylidene, or R¹⁰-substituted or unsubstituted C₃₋₄ cycloalkyl, or R¹⁰-substituted or unsubstituted 3 or 4-membered heterocycle; or wherein
two R⁹ together form a R¹⁰-substituted or unsubstituted C₃₋₅ cycloalkyl or a R¹⁰-substituted or unsubstituted C₃₋₅ heterocycle comprising one or more oxygen atoms
R¹⁰ is hydrogen, halogen, or C₁₋₃ unsubstituted alkyl;
each R^{8A} is independently R^{9A}-substituted or unsubstituted C₁₋₃ alkyl, R^{9A}-substituted or unsubstituted C₁₋₃ alkoxy, R^{9A}-substituted or unsubstituted C₃₋₄ cycloalkyl, or R^{9A}-substituted or unsubstituted 4-6 membered heterocycle;
each R^{9A} is independently halogen, oxo, unsubstituted C₁₋₃ alkyl, unsubstituted C₁₋₃ haloalkyl, unsubstituted C₁₋₃ alkoxy, unsubstituted C₁₋₃ alkylidene, R⁹-substituted or unsubstituted C₃₋₄ cycloalkyl, or R⁹-substituted or unsubstituted 4-10 membered heterocycle comprising N, S, or O;
R^{8B} is independently halogen, oxo, —NH₂, unsubstituted C₁₋₃ alkyl, unsubstituted C₁₋₃ haloalkyl, unsubstituted C₁₋₃ alkoxy, or unsubstituted C₁₋₃ alkylidene;
R³ and R⁴ are each independently hydrogen, —CN, halogen, unsubstituted C₁₋₃ alkyl, or unsubstituted cyclopropyl;
each R⁵ is independently halogen, oxo, unsubstituted C₁₋₃ alkyl, or unsubstituted C₁₋₃ haloalkyl; or wherein;
two R⁵ together form a bridge between two carbon atoms of ring A, wherein the bridge comprises 1-3 carbons and optionally one heteroatom selected from O and N; or
two R⁵ together form a bridge between two carbon atoms of ring A, wherein the bridge comprises one of O or NR¹¹;
R¹¹ is hydrogen, C(O)CH₃, or unsubstituted C₁₋₃ alkyl; and
R⁶ is hydrogen or R^{6A}-substituted or unsubstituted C₁₋₆ alkyl, R^{6A}-substituted or unsubstituted C₁₋₆ haloalkyl, R^{6A}-substituted or unsubstituted C₁₋₆ alkenyl; R^{6A}-substituted or unsubstituted C₁₋₆ alkynyl, or R^{6A}-substituted or unsubstituted 3-4 membered heterocycle;
R^{6A} is halogen, CN, OR^{6B}, SR^{6C}, S(O)₂R^{6C}, C(O)R^{6B}, unsubstituted C₁₋₃ alkyl; or, unsubstituted C₁₋₃ haloalkyl, R^{6B}-substituted or unsubstituted 3-4 membered heterocycle;
R^{6B} and R^{6C} are each independently C₁₋₃ alkyl or C₁₋₃ haloalkyl.

Embodiment 2. The compound of claim 1, wherein each R⁰ is hydrogen

Embodiment 3. The compound of claim 1, wherein one R⁰ is hydrogen and one R⁰ is methyl.

Embodiment 4. The compound of claim 3 having structure:

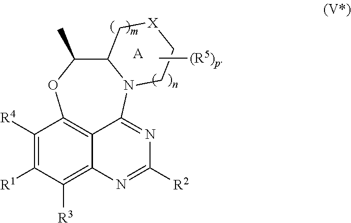

Embodiment 5. The compound of claim 1, wherein R¹ is R⁷-substituted or unsubstituted phenyl, R⁷-substituted or unsubstituted indazolyl, or R⁷-substituted or unsubstituted pyridinyl.

Embodiment 6. The compound of any one of claims 1-5, wherein R¹ is R⁷-substituted or unsubstituted phenyl.

Embodiment 7. The compound of any one of claims 1-5, wherein R¹ is R⁷-substituted or unsubstituted indazolyl.

Embodiment 8. The compound of any one of claims 1-5, wherein R¹ is R⁷-substituted or unsubstituted pyridinyl.

Embodiment 9. The compound of any one of claims 1-8, wherein each R⁷ is independently halogen, NH₂, unsubstituted C₁₋₃ alkyl, or unsubstituted C₁₋₃ haloalkyl.

Embodiment 10. The compound of any one of claims 1-5, wherein R¹ is

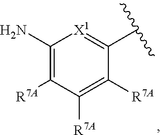

wherein,
X¹ is N or CF, and
R^{7A} is hydrogen, halogen, unsubstituted C₁₋₃ alkyl, or unsubstituted C₁₋₃ haloalkyl.

Embodiment 11. The compound of any one of claims 1, 5, 8, or 10, wherein R¹ is

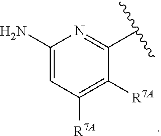

Embodiment 12. The compound of any one of claims 1, 5, 8, 10, or 11, wherein R¹ is

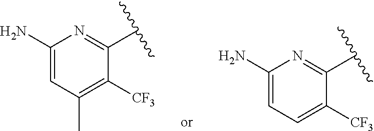 [SMILES: *c1nc(N)cc(C)c1C(F)(F)F ; *c1nc(N)ccc1C(F)(F)F]

Embodiment 13. The compound of any one of claims 1-6 or 10, wherein R¹ is

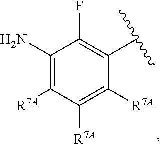

wherein R⁷ is hydrogen, halogen, unsubstituted C₁₋₃ alkyl or unsubstituted C₁₋₃ haloalkyl.

Embodiment 14. The compound of any one of claims 1-6, 10, or 13, wherein R¹ is

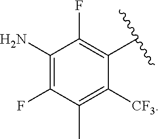 [SMILES: *c1c(F)c(N)c(F)c(C)c1C(F)(F)F]

Embodiment 15. The compound of any one of claims 1-5, wherein R¹ is

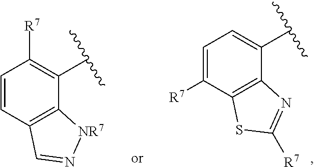

wherein each R⁷ is independently halogen, NH₂, N(Me)₂, or unsubstituted C₁₋₃ alkyl.

Embodiment 16. The compound of any one of claims 1-15, wherein R² is L¹-O-L²-R⁸, R^{8A}-substituted or unsubstituted C₁₋₃ alkyl, or R^{8B}-substituted or unsubstituted 4-6 membered heterocycle.

Embodiment 17. The compound of any one of claims 1-16, wherein R² is L¹-O-L²-R⁸.

Embodiment 18. The compound of any one of claims 1-17, wherein L¹ is a bond.

Embodiment 19. The compound of any one of claims 16-18, wherein L² is unsubstituted C₁₋₃ alkylene.

Embodiment 20. The compound of any one of claims 16-19, wherein R⁸ is 4-10 membered heterocycle comprising one N heteroatom.

Embodiment 21. The compound of any one of claims 16-20, wherein R⁸ is

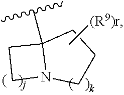

wherein,
R⁹ is halogen or R¹⁰-substituted or unsubstituted C₁₋₃ alkylidene
r is an integer of 0-12;
j is 1, 2, or 3; and
k is 1 or 2.

Embodiment 22. The compound of claim 21, wherein r is 0, 1, 2, or 3.

Embodiment 23. The compound of any one of claims 16-22, wherein R⁸ is

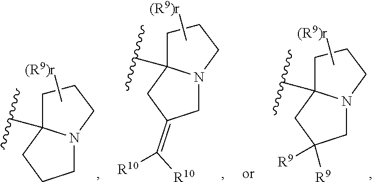

wherein,
R⁹ is independently halogen or R¹⁰-substituted or unsubstituted C₁₋₃ alkylidene;
each R¹⁰ is independently hydrogen or halogen; and
r is 1 or 2.

Embodiment 24. The compound of any one of claims 16-20, wherein R⁸ is

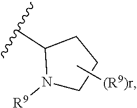

wherein,
R⁹ is independently halogen, oxo, or unsubstituted C₁₋₃ alkyl; and
r is 1 or 2.

Embodiment 25. The compound of any one of claims 16-20, wherein R⁸ is

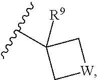

wherein
R⁹ is hydrogen or unsubstituted C₁₋₃ alkyl;
W is O, SO₂, or NR¹²; and R¹² is hydrogen, unsubstituted C₁₋₃ alkyl, or unsubstituted C₁₋₃ haloalkyl.

Embodiment 26. The compound of any one of claims 16-20 or 25, wherein R⁸ is azetidinyl, oxetanyl, or thietanedioxide.

Embodiment 27. The compound of any one of claims 1-26, wherein R² is

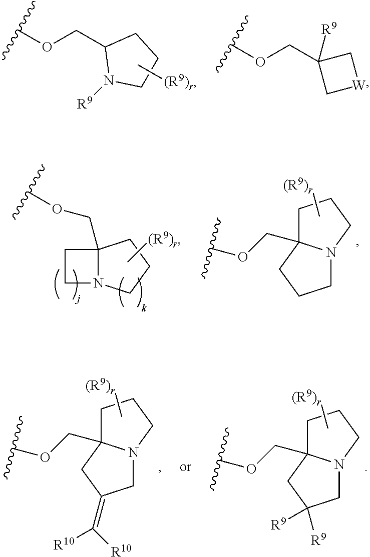

Embodiment 28. The compound of claim 27, wherein R⁹ is halogen or R¹⁰-substituted or unsubstituted C₁₋₃ alkylidene.

Embodiment 29. The compound of any one of claims 1-15, wherein R² is hydrogen.

Embodiment 30. The compound of any one of claims 1-16, wherein R² is R^{8A}-substituted or unsubstituted C₁₋₃ alkyl.

Embodiment 31. The compound of claim 29, wherein R^{8A} is independently R^{9A}-substituted or unsubstituted alkoxy or R^{9A}-substituted or unsubstituted 4-6 membered heterocycle.

Embodiment 32. The compound of claim 29 or claim 31, wherein R^{9A} is R⁹-substituted or unsubstituted 4-10 membered heterocycle comprising N.

Embodiment 33. The compound of claim 31 or 32, wherein R^{9A} is independently halogen, unsubstituted C₁₋₃ alkyl, or R¹⁰-substituted or unsubstituted C₁₋₃ alkylidene.

Embodiment 34. The compound of any one of claims 1-33, wherein R³ is halogen.

Embodiment 35. The compound of any one of claims 1-34, wherein R⁴ is hydrogen.

Embodiment 36. The compound of any one of claims 1-34, wherein R⁴ is halogen.

Embodiment 37. The compound of any one of claims 1-36, wherein R⁵ is independently oxo or unsubstituted C₁₋₃ alkyl, and p is 1.

Embodiment 38. The compound of any one of claims 1-36, wherein two R⁵ together form a bridge between two carbon atoms of ring A, wherein the bridge comprises 1-3 carbons.

Embodiment 39. The compound of claim 38, wherein the bridge comprises 1 carbon atom.

Embodiment 40. The compound of claim 38, wherein the bridge comprises 2 carbon atoms.

Embodiment 41. The compound of claim 1 having the formula:

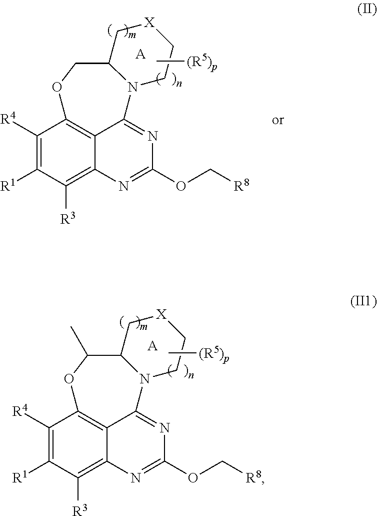

or a stereoisomer, atropisomer, tautomer, or pharmaceutically acceptable salt thereof.

Embodiment 42. The compound of claim 1 having the formula:

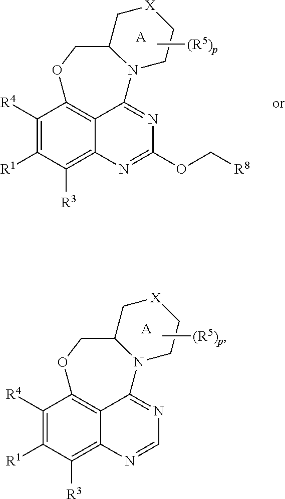

or a stereoisomer, atropisomer, tautomer, or pharmaceutically acceptable salt thereof.

Embodiment 43. The compound of claim 1 having the formula:

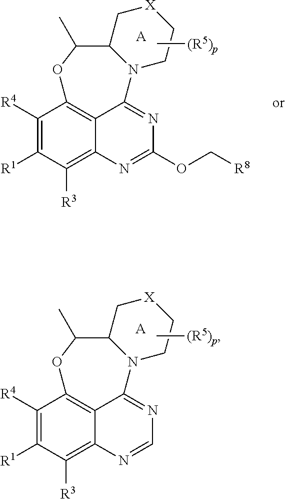

Embodiment 44. The compound of claim 1 having the formula:

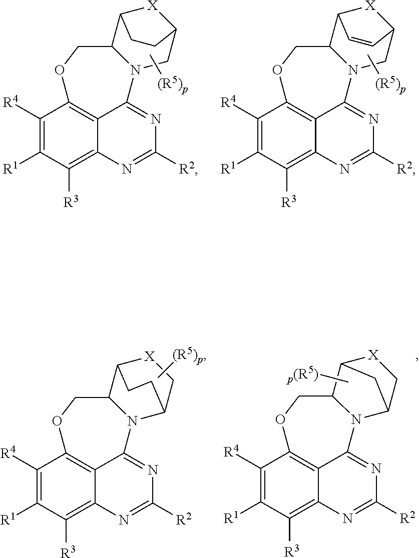

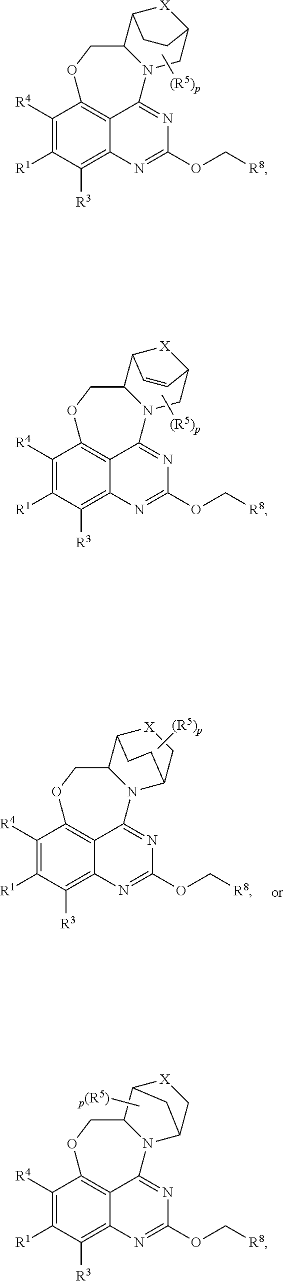

or a stereoisomer, atropisomer, tautomer, or pharmaceutically acceptable salt thereof.

Embodiment 45. The compound of claim 1 having the formula:

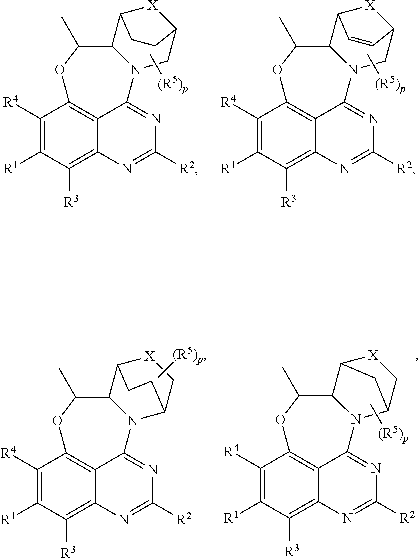

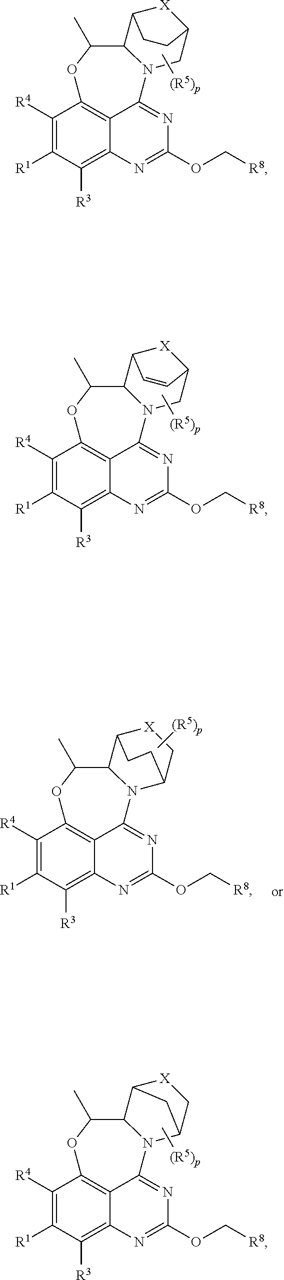

or a stereoisomer, atropisomer, tautomer, or pharmaceutically acceptable salt thereof.

Embodiment 46. The compound of claim 1 having the formula:

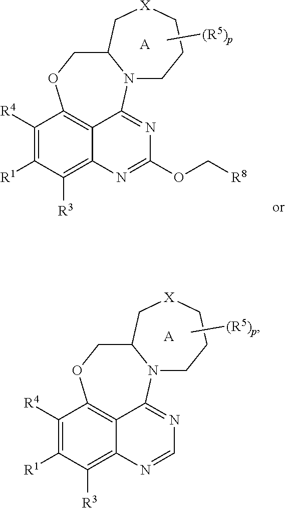

or a stereoisomer, atropisomer, tautomer, or pharmaceutically acceptable salt thereof.

Embodiment 47. The compound of claim 1 having the formula:

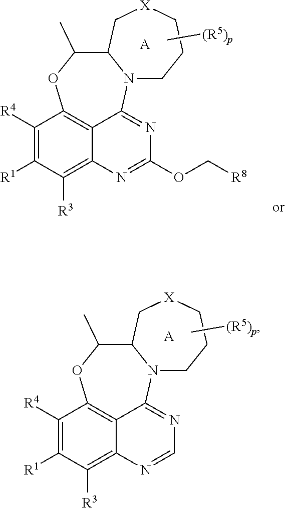

or a stereoisomer, atropisomer, tautomer, or pharmaceutically acceptable salt thereof.

Embodiment 48. The compound claim 1, having the formula:

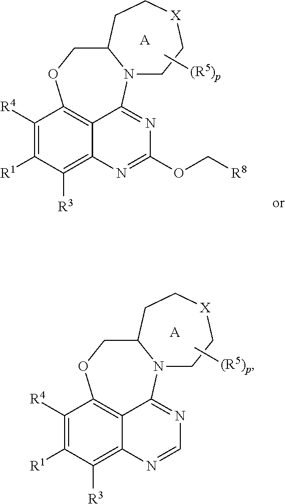

or a stereoisomer, atropisomer, tautomer, or pharmaceutically acceptable salt thereof.

Embodiment 49. The compound claim 1, having the formula:

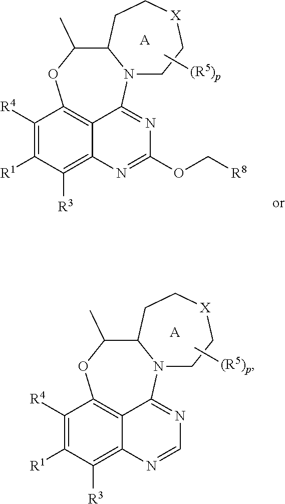

or a stereoisomer, atropisomer, tautomer, or pharmaceutically acceptable salt thereof.

Embodiment 50. The compound of any one of claims 1 or 41-49, wherein R⁸ is:

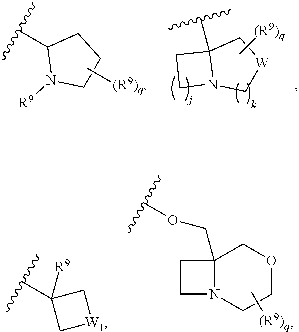

Embodiment 51. The compound of any one of claims 1 or 38-42, wherein R⁸ is:

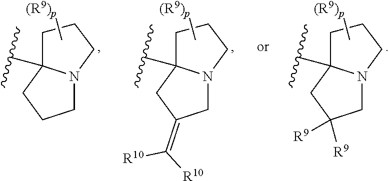

Embodiment 52. The compound of any one of claims 1 or 41-49, wherein R⁸ is:

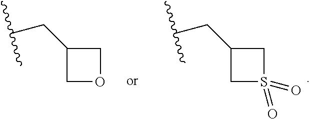 [SMILES: *CC1COC1 ; "or" ; *CC1CS(=O)(=O)C1 ; "."]

Embodiment 53. The compound of any one of claims 1-52, wherein X is NR⁶.

Embodiment 54. The compound claim 53, wherein R⁶ is R^{6A}-substituted or unsubstituted C₁₋₃ alkyl.

Embodiment 55. The compound claim 53, wherein R⁶ is R^{6A}-substituted C₁₋₃ alkyl.

Embodiment 56. The compound of claim 55, wherein R^{6A} is halogen, CN, OH, OMe, OEt, OCF₃, SO₂Me, unsubstituted C₁₋₃ alkyl, or 4-membered heterocycle.

Embodiment 57. The compound claim 53, wherein R⁶ is R^{6A}-substituted or unsubstituted C₁₋₃ haloalkyl.

Embodiment 58. The compound claim 53, wherein R⁶ is R^{6A}-substituted or unsubstituted C₁₋₃ alkenyl.

Embodiment 59. The compound claim 53, wherein R⁶ is R^{6A}-substituted or unsubstituted C₁₋₃ alkynyl.

Embodiment 60. The compound claim 53, wherein R⁶ is hydrogen.

Embodiment 61. The compound of claim 53, wherein R⁶ is methyl.

Embodiment 62. A compound of Table 1 or a stereoisomer, atropisomer, tautomer, or pharmaceutically acceptable salt thereof.

Embodiment 63. A compound of Table 2 or a stereoisomer, atropisomer, tautomer, or pharmaceutically acceptable salt thereof.

Embodiment 64. A compound of Table 3 or a stereoisomer, atropisomer, tautomer, or pharmaceutically acceptable salt thereof.

Embodiment 65. A pharmaceutical composition comprising a compound or a stereoisomer, atropisomer, tautomer, or pharmaceutically acceptable salt thereof of any one of claims 1-64 and one or more pharmaceutically acceptable excipients.

Embodiment 66. A method of treating cancer, the method comprising administering an effective amount of a compound or a stereoisomer, atropisomer, tautomer, or pharmaceutically acceptable salt thereof of any one of claims 1-64 or a pharmaceutical composition of claim 65.

Embodiment 67. The method of claim 66, wherein the cancer is characterized as comprising a KRas mutation.

Embodiment 68. The method of claim 67, wherein the KRas mutation corresponds to a KRas^{G12D} mutation.

Embodiment 69. The method of claim 68, further comprising testing a sample from the patient before administration for the absence or presence of a KRas^{G12D} mutation.

Embodiment 70. The method of claim 69, wherein the compound, stereoisomer, atropisomer, tautomer, or pharmaceutically acceptable salt thereof or pharmaceutical composition is administered to the patient after the patient sample shows the presence of a KRas^{G12D} mutation.

Embodiment 71. The method of any one of claims 66-70, wherein the cancer is tissue agnostic.

Embodiment 72. The method of any one of claims 66-70, wherein the cancer is pancreatic cancer, lung cancer, or colorectal cancer.

Embodiment 73. The method of claim 72, wherein the lung cancer is lung adenocarcinoma, NSCLC, or SCLC.

Embodiment 74. The method of claim 72, wherein the cancer is pancreatic cancer.

Embodiment 75. The method of claim 72, wherein the cancer is colorectal cancer.

Embodiment 76. The method of any one of claims 66-75, further comprising administering at least one additional therapeutic agent.

Embodiment 77. The method of claim 76, wherein the additional therapeutic agent comprises an epidermal growth factor receptor (EGFR) inhibitor, phosphatidylinositol kinase (PI3K) inhibitor, insulin-like growth factor receptor (IGF1R) inhibitor, a Janus kinase (JAK) inhibitor, a Met kinase inhibitor, a SRC family kinase inhibitor, a mitogen-activated protein kinase (MEK) inhibitor, an extracellular-signal-regulated kinase (ERK) inhibitor, a topoisomerase inhibitor, a taxane, an anti-metabolite agent, or an alkylating agent.

Embodiment 78. A compound according to any one of claims 1-64, or a stereoisomer, atropisomer, tautomer, or pharmaceutically acceptable salt thereof, for use as therapeutically active substance.

Embodiment 79. The use of a compound according to any one of claims 1-64, or stereoisomer, atropisomer, tautomer, or pharmaceutically acceptable salt thereof, for the therapeutic treatment of a cancer comprising a KRas^{G12D} mutation.

Embodiment 80. The use of a compound according to any one of claims 1-64, or stereoisomer, atropisomer, tautomer, or pharmaceutically acceptable salt thereof, for the preparation of a medicament for the therapeutic treatment of a cancer comprising a KRas^{G12D} mutation.

Embodiment 81. Use of a compound of any one of claims 1-64, or stereoisomer, atropisomer, tautomer, or pharmaceutically salt thereof, in the manufacture of a medicament for inhibiting tumor metastasis.

Embodiment 82. A compound according to any one of claims 1-64, or stereoisomer, atropisomer, tautomer, or pharmaceutically salt thereof, for the therapeutic and/or prophylactic treatment of a cancer comprising a KRas^{G12D} mutation.

Embodiment 83. A method for regulating activity of a KRas mutant protein, the method comprising reacting the mutant protein with a compound of any one of claims 1-64, or stereoisomer, atropisomer, tautomer, or pharmaceutically acceptable salt thereof.

Embodiment 84. A method for inhibiting proliferation of a cell population, the method comprising contacting the cell population with the compound of any one of claims 1-64, or stereoisomer, atropisomer, tautomer, or pharmaceutically acceptable salt thereof.

Embodiment 85. The method of claim 84, wherein the inhibition of proliferation is measured as a decrease in cell viability of the cell population.

Embodiment 86. A method for preparing a labeled KRas^{G12D} mutant protein, the method comprising reacting a KRas^{G12D} mutant protein with a labeled compound of any one of claims 1-64, or stereoisomer, atropisomer, tautomer, or pharmaceutically acceptable salt thereof, to result in the labeled KRas^{G12D} mutant protein.

Embodiment 87. A method for inhibiting tumor metastasis comprising administering to an individual in need thereof a therapeutically effective amount of the compound of any one of claims 1-64, or stereoisomer, atropisomer, tautomer, or pharmaceutically acceptable salt thereof or a pharmaceutical composition of claim 65 to a subject in need thereof.

Embodiment 88. A process for synthesizing a compound of formula (I) as set forth herein.

### EXAMPLES

### Intermediate 1: tert-Butyl (1R,2S,5S)-2-(hydroxymethyl)-3,8-diazabicyclo[3.2.1]octane-8-carboxylate

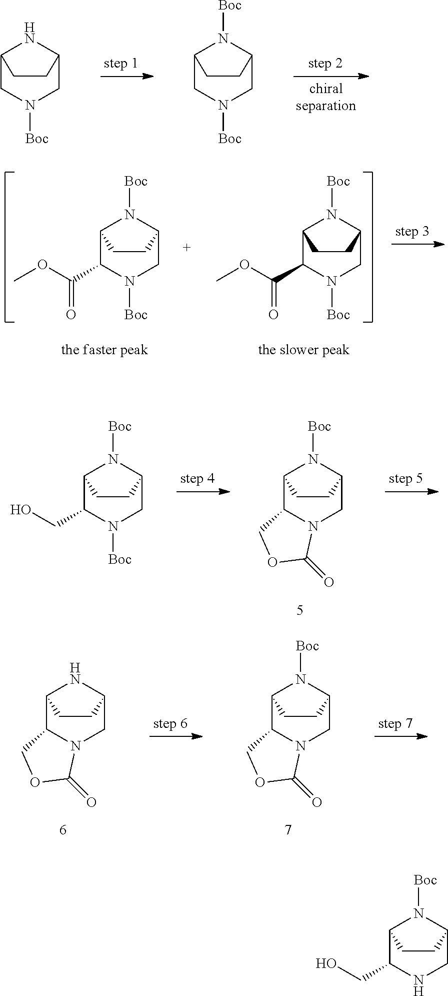

### Step 1: tert-Butyl (1R,5S)-8-benzyl-3,8-diazabicyclo[3.2.1]octane-3-carboxylate

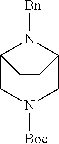 [SMILES: CC(C)(C)OC(=O)N1CC2CCC(C1)N2Cc1ccccc1]

To a solution of tert-butyl 3,8-diazabicyclo[3.2.1]octane-3-carboxylate (50.0 g, 236 mmol) and K₂CO₃ (65.1 g, 472 mmol) in N,N-dimethylformamide (800 mL) was added BnBr (60.1 g, 354 mmol) at 0° C. The reaction mixture was warmed to rt. After 2 h, ice water (1000 mL) was added. The resulting mixture was extracted with EtOAc (3×). The combined organic phases were washed with brine, dried over Na₂SO₄, and concentrated under vacuum. The residue was purified by flash chromatography on silica gel eluting with EtOAc/petroleum ether (0-10%) to afford the title compound (69 g, 97% yield) as a yellow oil. LC-MS: (ESI, m/z): [M+H]⁺=303.

### Step 2: 3-(tert-Butyl) 2-methyl (1R,2S,5S)-8-benzyl-3,8-diazabicyclo[3.2.1]octane-2,3-dicarboxylate and 3-(tert-butyl) 2-methyl (1R,2R,5S)-8-benzyl-3,8-diazabicyclo[3.2.1]octane-2,3-dicarboxylate

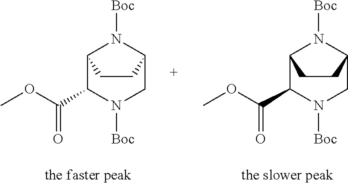 [SMILES: COC(=O)[C@@H]1[C@@H]2CC[C@H](CN1C(=O)OC(C)(C)C)N2C(=O)OC(C)(C)C ; COC(=O)[C@H]1[C@H]2CC[C@@H](CN1C(=O)OC(C)(C)C)N2C(=O)OC(C)(C)C]

To a solution of tert-butyl 8-benzyl-3,8-diazabicyclo[3.2.1]octane-3-carboxylate (23.0 g, 76.1 mmol) and TMEDA (17.7 g, 153 mmol) in diethyl ether (500 mL) was added dropwise s-BuLi (1.3 M in hexane) (117 mL, 152 mmol) at −78° C. under nitrogen. After 1.5 h, a solution of methyl chloroformate (17.9 g, 189 mmol) in 40 mL of Et₂O was added at −78° C. The reaction was warmed to room temperature. After 16 h, the reaction was quenched with saturated aqueous NaHCO₃ solution and diluted with 500 mL of water. The resulting mixture was extracted with EtOAc (3×). The combined organic phases were dried over Na₂SO₄ and concentrated under vacuum. The crude product was purified by flash chromatography on silica gel eluting with EtOAc/petroleum ether (0-10%) to afford 16 g of product (mixture of cis) as yellow oil, which contained ˜10% starting material tert-butyl 8-benzyl-3,8-diazabicyclo[3.2.1]octane-3-carboxylate co-eluting with the product. The mixture was separated by chiral-SFC (Column: Lux® 5 μm Cellulose-2, 5×25 cm, 5 μm; Mobile Phase A: CO₂, Mobile Phase B: MeCOH (0.1% 2M NH₃-MeOH); Flow rate:180 mL/min; Gradient:18% B; 220 nm; RT₁:5.07; RT₂: 5.57) to afford 5.9 g of the faster peak (isomer 1) and 5.6 g of the slower peak (isomer 2) as yellow oil. LC-MS: (ESI, m/z): [M+H]⁺=361.

### Step 3: tert-Butyl (1R,2S,5S)-8-benzyl-2-(hydroxymethyl)-3,8-diazabicyclo[3.2.1]octane-3-carboxylate

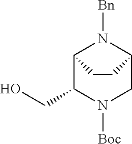 [SMILES: CC(C)(C)OC(=O)N1C[C@H]2CC[C@@H]([C@H]1CO)N2Cc1ccccc1]

To a solution of 3-(tert-butyl) 2-methyl (1R,2S,5S)-8-benzyl-3,8-diazabicyclo[3.2.1]octane-2,3-dicarboxylate (20.0 g, 55.5 mmol) in tetrahydrofuran (300 mL) cooled with ice salt bath was added LiAlH₄ (4.20 g, 111 mmol) portionwise under nitrogen at the rate to maintain reaction temperature below 5° C. The resulting solution was stirred for 30 mins at 0° C. The reaction was quenched with Na₂SO₄·10H₂O and filtered. The organic was concentrated under vacuum. The residue was purified by flash chromatography on silica gel eluting with EtOAc/petroleum ether (0-20%) to afford the title compound (14.3 g, 77.5% yield) as a white solid. LC-MS: (ESI, m/z): [M+H]⁺=333.

### Step 4: (6S,9R,9aS)-10-Benzylhexahydro-1H,3H-6,9-epiminooxazolo[3,4-a]azepin-3-one

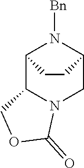 [SMILES: O=C1OC[C@@H]2[C@@H]3CC[C@H](CN12)N3Cc1ccccc1]

NaH (1.35 g, 33.8 mmol) was added portionwise to a solution of tert-butyl 8-benzyl-4-(hydroxymethyl)-3,8-diazabicyclo[3.2.1]octane-3-carboxylate (5.10 g, 15.3 mmol) in tetrahydrofuran (100 mL) at 0° C. The resulting suspension was warmed to rt. After 3 h, the reaction mixture was quenched with saturated aqueous NH₄Cl solution (30 mL). The resulting solution was extracted with EtOAc (3×). The combined organic layers were dried over anhydrous sodium sulfate and concentrated under vacuum. The residue was purified by flash chromatography on silica gel eluting with EtOAc/petroleum ether (0-40%) to afford the title compound (3.5 g, 88% yield) as a white solid. LC-MS: (ESI, m/z): [M+H]⁺=259.

### Step 5: (6S,9R,9aS)-Hexahydro-1H,3H-6,9-epiminooxazolo[3,4-a]azepin-3-one

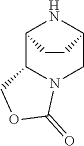 [SMILES: O=C1OC[C@@H]2[C@@H]3CC[C@H](CN12)N3]

To a solution of (6S,9R,9aS)-10-Benzylhexahydro-1H,3H-6,9-epiminooxazolo[3,4-a]azepin-3-one (10.0 g, 38.7 mmol) in methanol (200 mL) was added Pd/C (3.0 g, 10% dry) at rt. The resulting solution was stirred for 2 h under hydrogen. The suspension was filtered, and the filtrate was concentrated to afford 6 g of crude product which was used without further purification. LC-MS: (ESI, m/z): [M+H]⁺=169.

### Step 6: tert-Butyl (6S,9R,9aS)-3-oxohexahydro-1H,3H-6,9-epiminooxazolo[3,4-a]azepine-10-carboxylate

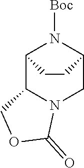 [SMILES: CC(C)(C)OC(=O)N1[C@@H]2CC[C@H]1[C@H]1COC(=O)N1C2]

To a solution of (6S,9R,9aS)-Hexahydro-1H,3H-6,9-epiminooxazolo[3,4-a]azepin-3-one (6.00 g, 35.7 mmol) and (Boc)₂O (12.6 g, 57.8 mmol) in dichloromethane (100 mL) was added DIPEA (10.0 g, 77.5 mmol) at 0° C. The solution was warmed to room temperature for 2 h. The solution was washed with saturated aqueous sodium chloride solution. The separated organic phase was concentrated under vacuum. The residue was purified by flash chromatography on silica gel eluting with EtOAc/petroleum ether (0-40%) to afford the title compound (7.50 g, 78.4% yield) as white solid. LC-MS: (ESI, m/z): [M+H]⁺=269.

### Step 7: tert-Butyl (1R,2S,5S)-2-(hydroxymethyl)-3,8-diazabicyclo[3.2.1]octane-8-carboxylate

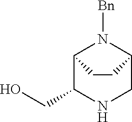 [SMILES: OC[C@H]1NC[C@H]2CC[C@@H]1N2Cc1ccccc1]

To a solution of tert-Butyl (6S,9R,9aS)-3-oxohexahydro-1H,3H-6,9-epiminooxazolo[3,4-a]azepine-10-carboxylate (7.50 g, 28.0 mmol) in ethanol (200 mL) was added NaOH (16.8 g, 420 mmol) in water (70 mL). The resulting solution was heated at 80° C. for 16 h. EtOH was removed under reduced pressure and the resulting aqueous solution was neutralized to pH-8 with HCl (1 M). The solution was extracted with EtOAc (3×). The combined organic layers were dried over Na₂SO₄ and concentrated under reduced pressure. The residue was purified by flash chromatography on silica gel eluting with DCM/MeOH (5/1) to afford the title compound (5.0 g, 74% yield) as an off white solid. LC-MS: (ESI, m/z): [M+H]⁺=243. ¹H NMR (400 MHz, DMSO-d₆, ppm) δ 4.72-4.57 (m, 1H), 4.02-3.90 (m, 2H), 3.25-3.15 (m, 2H), 2.82-2.68 (m, 2H), 2.64-2.53 (m, 1H), 1.85-1.61 (m, 3H), 1.61-1.47 (m, 1H), 1.41 (s, 9H).

### Intermediate 2: 7-Bromo-2,6-dichloro-5,8-difluoroquinazolin-4 (3H)-one

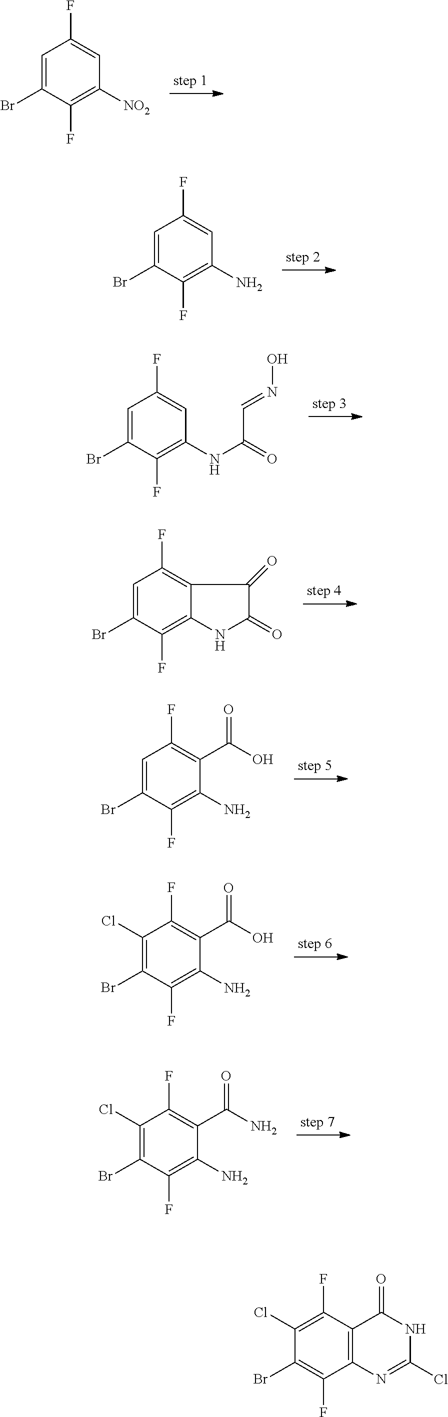

### Step 1: 3-Bromo-2,5-difluoroaniline

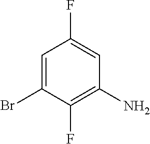

To a suspension of 1-bromo-2,5-difluoro-3-nitrobenzene (40.0 g, 168 mmol) and iron powder (28.4 g, 506 mmol) in water (10 mL) was added concentrated hydrochloric acid (40 mL, 36%) at room temperature. The suspension was heated to 100° C. After 1 h, the reaction system was cooled to room temperature and filtered. The filter cake was washed with EtOAc. The combined filtrates were concentrated under reduced pressure to afford the title compound (34.3 g, crude) as a brown oil which was used without further purification. LC-MS: (ESI, m/z): [M+H]⁺=208.

### Step 2: N-(3-Bromo-2,5-difluorophenyl)-2-(hydroxyimino)acetamide

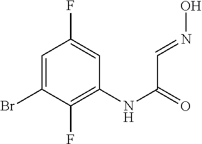 [SMILES: O=C(/C=N/O)Nc1cc(F)cc(Br)c1F]

To a solution of 2,2,2-trichloroethane-1,1-diol (40.9 g, 247 mmol), Na₂SO₄ (187 g, 1.32 mol) and NH₂OH·HCl (39.8 g, 577 mmol) in water (680 mL) was added a solution of 3-bromo-2,5-difluoroaniline (34.3 g, 165 mmol) in ethanol (100 mL), hydrochloric acid (12.5 mL, 36%) and water (50 ml). The resulting solution was heated at 60° C. for 3 h. The reaction system was cooled to room temperature and filtered. The solid was collected and rinsed with water (500 mL), dried in an oven to afford the title compound (32.8 g, crude) as a light brown solid which was used without further purification. LC-MS: (ESI, m/z): [M+H]⁺=279.

### Step 3: 6-Bromo-4,7-difluoroindoline-2,3-dione

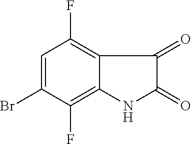 [SMILES: O=C1Nc2c(F)c(Br)cc(F)c2C1=O]

A solution of N-(3-bromo-2,5-difluorophenyl)-2-(hydroxyimino)acetamide (32.8 g, 118 mmol) in H₂SO₄ (160 mL, 98%) was heated at 90° C. for 1 h. The reaction mixture was cooled to room temperature and slowly added to ice water. The precipitate was collected by filtration, washed with water and dried in an oven to afford the title compound (28.1 g, crude) as a brown solid which was used without further purification. LC-MS: (ESI, m/z): [M+H]⁺=262.

### Step 4: 2-Amino-4-bromo-3,6-difluorobenzoic acid

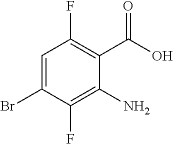 [SMILES: Nc1c(F)c(Br)cc(F)c1C(=O)O]

A solution of 6-bromo-4,7-difluoroindoline-2,3-dione (28.1 g, 107 mmol) in NaOH (537 mL, 2M in water) and H₂O₂ (53.7 mL, 30%) was stirred at room temperature for 16 h. The mixture was poured into ice water and adjusted to pH=2 with conc. HCl. The solid was collected by filtration and rinsed with water. The crude product was purified by reverse phase chromatography (gradient: 0-60% acetonitrile in water (0.1% formic acid)) to afford the title compound (13.4 g, 49.4% yield) as a light brown solid. LC-MS: (ESI, m/z): [M+H]⁺=252.

### Step 5: 2-Amino-4-bromo-5-chloro-3,6-difluorobenzoic acid

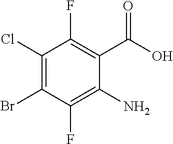 [SMILES: Nc1c(F)c(Br)c(Cl)c(F)c1C(=O)O]

A solution of 2-amino-4-bromo-3,6-difluorobenzoic acid (10.9 g, 43.2 mmol) and N-chlorosuccinimide (6.32 g, 47.5 mmol) in DMF (100 mL) was stirred at 90° C. for 1 h. The mixture was cooled to room temperature and poured into water (500 mL). The solid was collected and dried in an oven to afford the title compound (12.1 g, crude) as a brown solid which was used without further purification LC-MS: (ESI, m/z): [M+H]⁺=286.

### Step 6: 2-Amino-4-bromo-5-chloro-3,6-difluorobenzamide

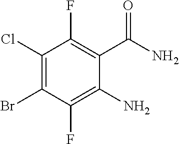 [SMILES: NC(=O)c1c(N)c(F)c(Br)c(Cl)c1F]

To a solution of 2-amino-4-bromo-5-chloro-3,6-difluorobenzoic acid (11.9 g, 41.6 mmol), NH₄Cl (4.42 g, 83.4 mmol) and DIPEA (13.5 g, 104 mmol) in DMF (60 mL) was added HATU (17.43 g, 45.84 mmol) at room temperature. The resulting solution was stirred for 30 min. The mixture was poured into water (300 mL), and the resulting precipitate was collected by filtration. The solid was suspended in EtOAc/petroleum ether (1:4, 100 mL) and stirred for 3 h. The solids were collected by filtration and dried in an oven to afford the title compound (8.85 g, crude) as a brown solid which was used without further purification. LC-MS: (ESI, m/z): [M+H]⁺=285.

### Step 7: 7-Bromo-2,6-dichloro-5,8-difluoroquinazolin-4 (3H)-one

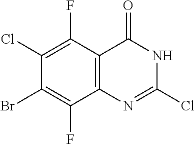 [SMILES: O=c1[nH]c(Cl)nc2c(F)c(Br)c(Cl)c(F)c12]

A solution of 2-amino-4-bromo-5-chloro-3,6-difluorobenzamide (8.85 g, 31.0 mmol) and thiophosgene (10.7 g, 93.2 mmol) in 1,4-dioxane (175 mL) was stirred at room temperature for 1 h. The mixture was then heated to 105° C. for 1 h. The reaction was concentrated under vacuum, and the residue was suspended in EtOAc/petroleum ether (1:4, 60 mL) and stirred for 1 h. The solids were collected by filtration and dried to afford the title compound (7.08 g, crude) as a brown solid which was used without further purification. LC-MS: (ESI, m/z): [M+H]⁺=330.

### Intermediate 3 tert-Butyl (5aS,6S,9R)-2-bromo-3,13-dichloro-1-fluoro-5a,6,7,8,9,10-hexahydro-5H-6,9-epiminoazepino[2′,1′:3,4][1,4]oxazepino[5,6,7-de]quinazoline-15-carboxylate

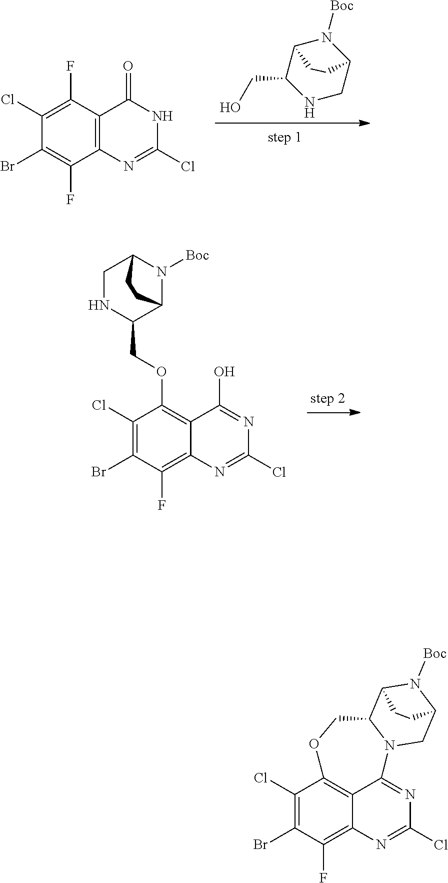

### Step 1: tert-Butyl (1 S,2S,5R)-2-(((7-bromo-2,6-dichloro-8-fluoro-4-hydroxyquinazolin-5-yl)oxy)methyl)-3,8-diazabicyclo[3.2.1]octane-8-carboxylate

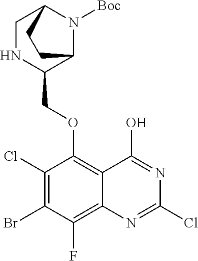 [SMILES: CC(C)(C)OC(=O)N1[C@@H]2CC[C@H]1[C@@H](COc1c(Cl)c(Br)c(F)c3nc(Cl)nc(O)c13)NC2]

To an ice-cooled solution of tert-butyl (1S,2S,5R)-2-(hydroxymethyl)-3,8-diazabicyclo[3.2.1]octane-8-carboxylate (2.30 g, 9.49 mmol, Intermediate 1) in tetrahydrofuran (100 mL) was added NaH (3.20 g, 80.0 mmol) under nitrogen. The resulting solution was warmed to room temperature. After 30 min, 7-bromo-2,6-dichloro-5,8-difluoroquinazolin-4-ol (4.40 g, 13.3 mmol, Intermediate 2) in tetrahydrofuran (50 mL) was added, and the reaction was stirred at room temperature for 2 h. The reaction was quenched with saturated aqueous NH₄Cl solution and diluted with 200 mL water. The resulting mixture was extracted with EtOAc. The combined organic extracts were dried over anhydrous sodium sulfate and concentrated under vacuum. The residue was purified by flash chromatography on silica gel (gradient: 0%-10% MeCOH/DCM) to yield 3.6 g (48% yield) of the title compound as a yellow solid. LC-MS: (ESI, m/z): [M+H]⁺=551/553.

### Step 2: tert-Butyl (5aS,6S,9R)-2-bromo-3,13-dichloro-1-fluoro-5a,6,7,8,9,10-hexahydro-5H-6,9-epiminoazepino[2′,1′:3,4][1,4]oxazepino[5,6,7-de]quinazoline-15-carboxylate

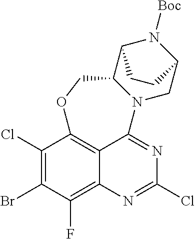 [SMILES: CC(C)(C)OC(=O)N1[C@@H]2CC[C@H]1[C@H]1COc3c(Cl)c(Br)c(F)c4nc(Cl)nc(c34)N1C2]

To a solution of tert-butyl (1 S,2S,5R)-2-(((7-bromo-2,6-dichloro-8-fluoro-4-hydroxyquinazolin-5-yl)oxy)methyl)-3,8-diazabicyclo[3.2.1]octane-8-carboxylate (8.00 g, 14.5 mmol) and BOP-Cl (13.5 g, 53.0 mmol) in dichloromethane (150 mL) was added DIPEA (28.0 g, 217 mmol). The resulting solution was stirred at room temperature for 5 h. The reaction mixture was washed with brine and the organic layer was concentrated under vacuum. The residue was purified by flash chromatography on silica gel (gradient: 0%-30% ethyl acetate/petroleum ether) and then slurry with ethyl acetate/petroleum ether=1:10 to afford 3.50 g (45% yield) of the title compound as a light-yellow solid. LC-MS: (ESI, m/z): [M+H]⁺=533/535. ¹H NMR (400 MHz, DMSO-d₆, ppm) δ 4.90-4.80 (m, 1H), 4.76-4.67 (m, 1H), 4.55-4.46 (m, 1H), 4.38-4.22 (m, 2H), 4.16-4.08 (m, 1H), 3.20-3.12 (m, 1H), 1.92-1.65 (m, 4H), 1.45 (s, 9H).

### Intermediate 4: 6-Bromo-N,N-bis(4-methoxybenzyl)-4-methyl-5-(trifluoromethyl)pyridin-2-amine

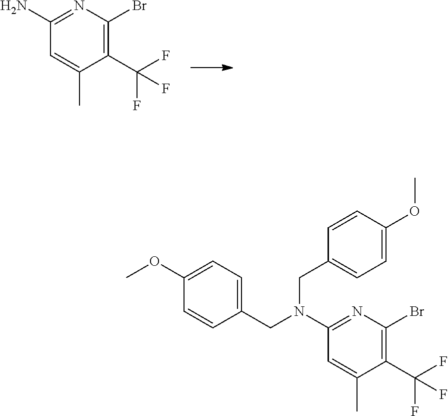 [SMILES: Cc1cc(N)nc(Br)c1C(F)(F)F ; COc1ccc(CN(Cc2ccc(OC)cc2)c2cc(C)c(C(F)(F)F)c(Br)n2)cc1]

To an ice-cooled solution of 6-bromo-4-methyl-5-(trifluoromethyl)pyridin-2-amine (14.0 g, 54.9 mmol) in N,N-dimethylformamide (300 mL) was added 60% NaH (6.58 g, 165 mmol) under an atmosphere of nitrogen. The resulting solution was stirred for 1 h at rt. Then PMB-Cl (21.4 g, 137 mmol) was added at 0° C. and stirred at rt for 1 h. The reaction was quenched with saturated ammonium chloride, extracted with EtOAc (3×500 mL). The combined organic layers were dried over anhydrous sodium sulfate and concentrated under vacuum. The residue was purified by flash chromatography on silica gel (gradient: 0-20% ethyl acetate/petroleum ether) to afford the title compound (23 g, 84.6% yield) as a white solid. LC-MS: (ESI, m/z): [M+H]⁺=495/497; ¹H NMR (400 MHz, DMSO-d₆, ppm) δ 7.19 (d, J=8.3 Hz, 4H), 6.93-6.85 (m, 4H), 6.65 (s, 1H), 4.67 (s, 4H), 3.73 (s, 6H), 2.31 (q, J=3.3 Hz, 3H).

### Intermediate 5: tert-Butyl (5aS,6S,9R)-2-((R)-6-(bis(4-methoxybenzyl)amino)-4-methyl-3-(trifluoromethyl)pyridin-2-yl)-3,13-dichloro-1-fluoro-5a,6,7,8,9,10-hexahydro-5H-6,9-epiminoazepino[2′,1′:3,4][1,4]oxazepino[5,6,7-de]quinazoline-15-carboxylate and tert-butyl (5aS,6S,9R)-2-((S)-6-(bis(4-methoxybenzyl)amino)-4-methyl-3-(trifluoromethyl)pyridin-2-yl)-3,13-dichloro-1-fluoro-5a,6,7,8,9,10-hexahydro-5H-6,9-epiminoazepino[2′,1′:3,4][1,4]oxazepino[5,6,7-de]quinazoline-15-carboxylate

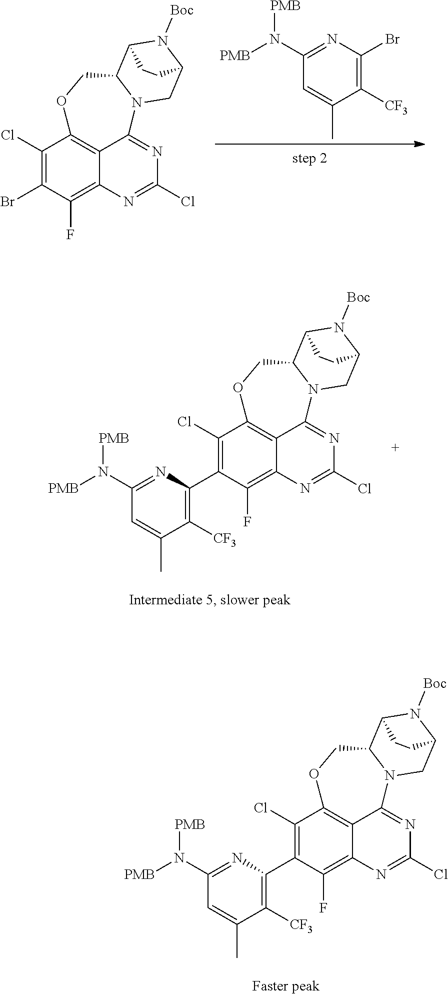

To a cold (−78° C.) solution of tert-butyl (5aS,6S,9R)-2-bromo-3,13-dichloro-1-fluoro-5a,6,7,8,9,10-hexahydro-5H-6,9-epiminoazepino[2′,1′:3,4][1,4]oxazepino[5,6,7-de]quinazoline-15-carboxylate (6.50 g, 12.2 mmol, Intermediate 3) in tetrahydrofuran (25 mL) under nitrogen was added i-PrMgCl·LiCl (9.86 mL, 12.8 mmol) dropwise at −78° C. The solution was stirred at −78° C. for 1 h, then ZnCl₂ (6.72 mL, 13.4 mmol) was added into the cold solution dropwise. The solution was stirred at −78° C. for 10 min, then the solution was slowly warmed to 25° C. and stirred for 1 h. The resulting solution was separated into 6 portions and each portion was transferred into the following solution (6-bromo-N,N-bis(4-methoxybenzyl)-4-methyl-5-(trifluoromethyl)pyridin-2-amine (905 mg, 1.83 mmol, Intermediate 4), and PdCl₂(PPh₃)₂ (57.1 mg, 0.0813 mmol) in tetrahydrofuran (4.6 mL)) separately under nitrogen. The reaction system was stirred overnight at 50° C., all 6 reactions were run in parallel. The combined reaction mixtures were diluted with water, extracted with EtOAc (3×). The organic layers were combined, dried over anhydrous sodium sulfate and concentrated under vacuum. The residue was purified by flash chromatography on silica gel (gradient: 0%-30% ethyl acetate/petroleum ether) to yield 3.10 g the title compound (29% yield, mixture of two atropisomers) as a yellow solid. The reaction was repeated and a total of 7.1 g racemic compound was obtained. The racemic mixture was separated by SFC-HPLC (Column: Lux 5 μm Cellulose-2, 3×15 cm, 5 μm; Mobile Phase A: CO₂, Mobile Phase B: MeCOH (0.1% 2M NH₃-MeOH); Flow rate: 70 mL/min; Gradient: 50% B to 50% B in 24 min; 254/220 nm; R_{T1}:11.46; R_{T2}:19.55) to yield 2.30 g (the slower peak, desired isomer) and 2.40 g (the faster peak) as yellow solids. And 1.3 g of mixture of atropsiomers was recovered. LC-MS: (ESI, m/z): [M+H]⁺=869.3. ¹H NMR (300 MHz, DMSO-d₆, ppm) δ 7.14 (d, J=8.4 Hz, 4H), 6.86 (d, J=8.7 Hz, 4H), 6.82 (s, 1H), 4.92-4.81 (m, 1H), 4.81-4.63 (m, 3H), 4.62-4.47 (m, 3H), 4.41-4.24 (m, 2H), 4.18-4.05 (m, 1H), 3.72 (s, 6H), 3.12 (d, J=13.2 Hz, 1H), 2.39 (s, 3H), 2.00-1.63 (m, 4H), 1.44 (s, 9H).

### Intermediate 6: 7-Bromo-2-chloro-5,6,8-trifluoroquinazolin-4 (3H)-one

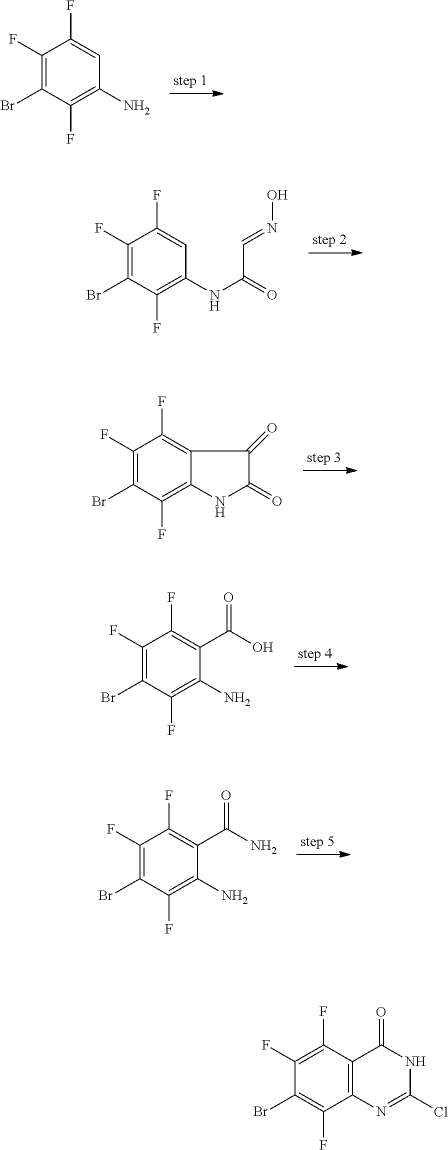 [SMILES: Nc1cc(F)c(F)c(Br)c1F ; O=C(/C=N/O)Nc1cc(F)c(F)c(Br)c1F ; O=C1Nc2c(F)c(Br)c(F)c(F)c2C1=O ; Nc1c(F)c(Br)c(F)c(F)c1C(=O)O ; NC(=O)c1c(N)c(F)c(Br)c(F)c1F ; O=c1[nH]c(Cl)nc2c(F)c(Br)c(F)c(F)c12]

### Step 1: (E)-N-(3-bromo-2,4,5-trifluorophenyl)-2-(hydroxyimino)acetamide

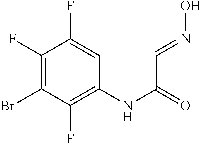 [SMILES: O=C(/C=N/O)Nc1cc(F)c(F)c(Br)c1F]

To a solution of Na₂SO₄ (207 g, 1438 mmol), hydroxylamine hydrochloride (44.7 g, 643.2 mmol) and CHLORALHYDRATE (46.2 g, 279.32 mmol) in water (1000 mL) was added a solution of 3-bromo-2,4,5-trifluoro-aniline (40.0 g, 177 mmol) in hydrochloric acid (30 mL, 37%), ethanol (80 mL) and water (100 mL). The resulting solution was stirred for 2 hours at 60° C. The precipitate was collected by filtration, washed with water, dried under vacuum to afford the title compound (43.2 g, 82.2% yield) as a light-brown solid. LC-MS: (ESI, m/z): [M−H]⁺=295.

### Step 2: 6-Bromo-4,5,7-trifluoroindoline-2,3-dione

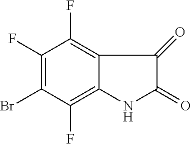 [SMILES: O=C1Nc2c(F)c(Br)c(F)c(F)c2C1=O]

A solution of (2E)-N-(3-bromo-2,4,5-trifluoro-phenyl)-2-hydroxyimino-acetamide (43.2 g, 145 mmol) in triflic acid (120 mL, 1356 mmol) was stirred for 3 hours at 130° C. The solution was cooled to room temperature and added to ice water (1.2 L) dropwise. The precipitate was collected by filtration, washed with petroleum ether/ethyl acetate (10:1) to afford 10 g of the title compound. The filtrate was extracted with EtOAc, the organic layer was washed with NaHCO₃ (sat.) and brine, dried over anhydrous sodium sulfate and concentrated under vacuum to afford another 7.8 g of the title compound. In total, 17.8 g (43.7% yield) of the tile compound was as a brown solid. No mass signal.

### Step 3: 2-Amino-4-bromo-3,5,6-trifluorobenzoic acid

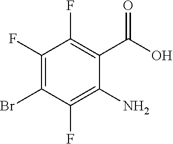 [SMILES: Nc1c(F)c(Br)c(F)c(F)c1C(=O)O]

To a solution of 6-bromo-4,5,7-trifluoro-indoline-2,3-dione (21.0 g, 75 mmol) in sodium hydroxide (2M in water) (400 mL) was added hydrogen peroxide (30% in water) (40 mL) dropwise with stirring. The solution was stirred for 16 hours at rt. The insoluble solid was filtered off. The filtrate was acidified to pH=2 with HCl (37% in water). The precipitate was collected by filtration, dried under vacuum to afford the title compound (11.8 g, 58.3% yield) as a brown solid. LC-MS: (ESI, m/z): [M+H]⁺=290.

### Step 4: 2-Amino-4-bromo-3,5,6-trifluorobenzamide

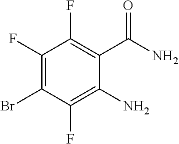 [SMILES: NC(=O)c1c(N)c(F)c(Br)c(F)c1F]

A solution of 2-amino-4-bromo-3,5,6-trifluoro-benzoic acid (11.8 g, 43.7 mmol), NH4Cl (9.4 g, 175.7 mmol), DIPEA (16.7 g, 130 mmol) and HATU (19.1 g, 50.2 mmol) in N,N-dimethylformamide (60 mL) was stirred for 2 hours at rt. The resulting solution was poured into water with stirring. The precipitate was collected by filtration and dried under vacuum to afford the title compound (6.1 g, 51.9% yield) as a brown solid. LC-MS: (ESI, m/z): [M+H]⁺=269.

### Step 5: 7-Bromo-2-chloro-5,6,8-trifluoroquinazolin-4 (3H)-one

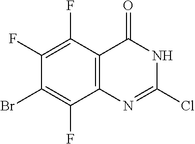 [SMILES: O=c1[nH]c(Cl)nc2c(F)c(Br)c(F)c(F)c12]

A solution of 2-amino-4-bromo-3,5,6-trifluoro-benzamide (6.1 g, 22.7 mmol) and thiophosgene (5.4 mL, 70.5 mmol) in 1,4-dioxane (120 mL) was stirred at rt for 1 hour, and heated at reflux for 1 hour. The solution was cooled to room temperature and concentrated under vacuum. The solid was washed with petroleum ether/ethyl acetate (4:1) to afford the title compound (5.9 g, 66.4% yield) as a brown solid. LC-MS: (ESI, m/z): [M+H]⁺=313.

### Intermediate 7: tert-Butyl (5aS,6S,9R)-2-bromo-13-chloro-1,3-difluoro-5a,6,7,8,9,10-hexahydro-5H-6,9-epiminoazepino[2′,1′:3,4][1,4]oxazepino[5,6,7-de]quinazoline-15-carboxylate

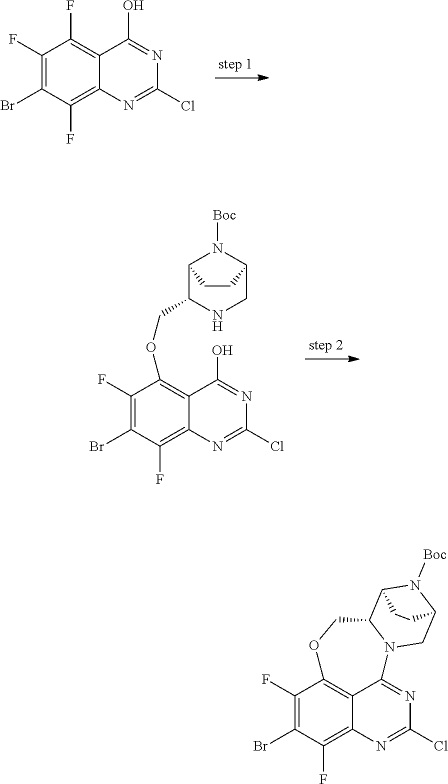 [SMILES: Oc1nc(Cl)nc2c(F)c(Br)c(F)c(F)c12 ; CC(C)(C)OC(=O)N1[C@@H]2CC[C@H]1[C@@H](COc1c(F)c(Br)c(F)c3nc(Cl)nc(O)c13)NC2 ; CC(C)(C)OC(=O)N1[C@@H]2CC[C@H]1[C@H]1COc3c(F)c(Br)c(F)c4nc(Cl)nc(c34)N1C2]

### Step 1: tert-Butyl (1R,2S,5S)-2-(((7-bromo-2-chloro-6,8-difluoro-4-hydroxyquinazolin-5-yl)oxy)methyl)-3,8-diazabicyclo[3.2.1]octane-8-carboxylate

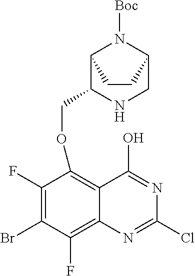 [SMILES: CC(C)(C)OC(=O)N1[C@@H]2CC[C@H]1[C@@H](COc1c(F)c(Br)c(F)c3nc(Cl)nc(O)c13)NC2]

Under nitrogen, to a solution of tert-butyl (1S,2S,5R)-2-(hydroxymethyl)-3,8-diazabicyclo[3.2.1]octane-8-carboxylate (695 mg, 2.87 mmol, Intermediate 1) in THF (10 mL) was added NaH (382 mg, 9.57 mmol). The reaction was stirred at room temperature for 15 mins. Then 7-bromo-2-chloro-5,6,8-trifluoro-quinazolin-4-ol (1.00 g, 3.19 mmol, Intermediate 6) was added at 0° C. and stirred at 0° C. for 2 hours. Then acetic acid was added to quench the reaction. The resulting mixture was partitioned between water and DCM. Organic layers were combined, dried over anhydrous Na₂SO₄, concentrated under vacuum. The residue was purified by reverse phase flash chromatography (gradient: 0-100% ACN in H₂O (0.05% NH₄HCO₃)) to afford 910 mg (crude) of the title compound as a brown solid. LC-MS: (ESI, m/z): [M+H]⁺=535.

### Step 2: tert-Butyl (5aS,6S,9R)-2-bromo-13-chloro-1,3-difluoro-5a,6,7,8,9,10-hexahydro-5H-6,9-epiminoazepino[2′,1′:3,4][1,4]oxazepino[5,6,7-de]quinazoline-15-carboxylate

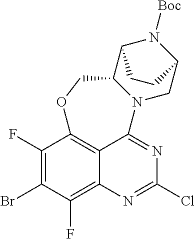 [SMILES: CC(C)(C)OC(=O)N1[C@@H]2CC[C@H]1[C@H]1COc3c(F)c(Br)c(F)c4nc(Cl)nc(c34)N1C2]

A solution of tert-butyl (1 S,2S,5R)-2-[(7-bromo-2-chloro-6,8-difluoro-4-hydroxy-quinazolin-5-yl)oxymethyl]-3,8-diazabicyclo[3.2.1]octane-8-carboxylate (915 mg, 1.71 mmol), DIPEA (3.31 g, 25.6 mmol) and BOP-Cl (1.31 g, 5.12 mmol) in DCM (10 mL) was stirred at room temperature for 3 h. The resulting mixture was partitioned between water and DCM. The collected organic layer was dried over anhydrous Na₂SO₄ and concentrated under vacuum. The residue was purified by flash chromatography on silica gel (gradient: 0-30% EtOAc in petroleum ether) to afford a white solid (560 mg, 63% yield). LC-MS: (ESI, m/z): [M+H]⁺=517. ¹H NMR (300 MHz, DMSO-d₆) δ 4.89-4.78 (m, 1H), 4.70 (dd, J=13.4, 2.8 Hz, 1H), 4.50 (dd, J=13.2, 7.3 Hz, 1H), 4.39-4.24 (m, 2H), 4.10 (d, J=7.0 Hz, 1H), 3.16 (d, J=13.3 Hz, 1H), 1.89-1.76 (m, 4H), 1.45 (s, 9H).

### Intermediate 8: tert-Butyl (5aS,6S,9R)-2-bromo-3,13-dichloro-5a,6,7,8,9,10-hexahydro-5H-6,9-epiminoazepino[2′,1′:3,4][1,4]oxazepino[5,6,7-de]quinazoline-15-carboxylate

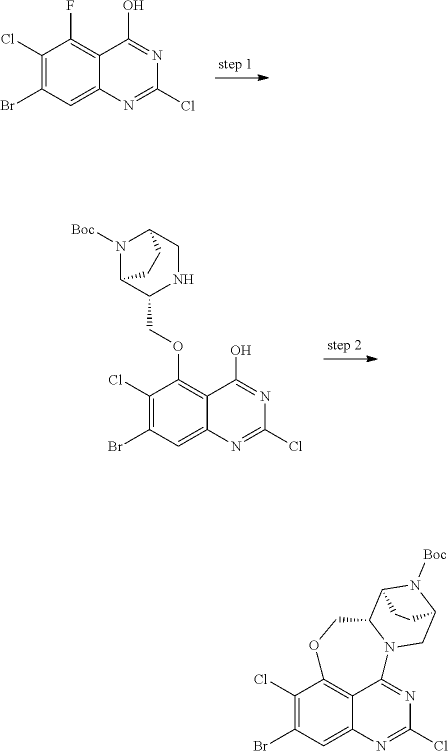 [SMILES: Oc1nc(Cl)nc2cc(Br)c(Cl)c(F)c12 ; CC(C)(C)OC(=O)N1[C@@H]2CC[C@H]1[C@@H](COc1c(Cl)c(Br)cc3nc(Cl)nc(O)c13)NC2 ; CC(C)(C)OC(=O)N1[C@@H]2CC[C@H]1[C@H]1COc3c(Cl)c(Br)cc4nc(Cl)nc(c34)N1C2]

### Step 1: tert-Butyl (1S,2S,5R)-2-(((7-bromo-2,6-dichloro-4-hydroxyquinazolin-5-yl)oxy)methyl)-3,8-diazabicyclo[3.2.1]octane-8-carboxylate

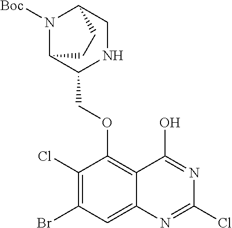 [SMILES: CC(C)(C)OC(=O)N1[C@@H]2CC[C@H]1[C@@H](COc1c(Cl)c(Br)cc3nc(Cl)nc(O)c13)NC2]

To an ice-cooled solution of tert-butyl (1S,2S,5R)-2-(hydroxymethyl)-3,8-diazabicyclo[3.2.1]octane-8-carboxylate (463 mg, 1.91 mmol, Intermediate 1) in tetrahydrofuran (10 mL) was added NaH (191 mg, 4.78 mmol, 60% in mineral oil) under nitrogen. After 0.5 h, 7-bromo-2,6-dichloro-5-fluoroquinazolin-4-ol (0.500 g, 1.61 mmol) was added, and the reaction mixture was heated to 65° C. for 2 h. The reaction was quenched with saturated aqueous NH₄Cl solution and extracted with DCM. The organic layers were combined, dried over anhydrous sodium sulfate, and concentrated under vacuum. The residue was purified by flash chromatography on silica gel (gradient: 0-10% methanol/DCM) to yield 460 mg (53% yield) of the title compound as a yellow solid. LC-MS: (ESI, m/z): [M+H]+=533.

### Step 2: tert-Butyl (5aS,6S,9R)-2-Bromo-3,13-dichloro-5a,6,7,8,9,10-hexahydro-5H-6,9-epiminoazepino[2′,1′:3,4][1,4]oxazepino[5,6,7-de]quinazoline-15-carboxylate

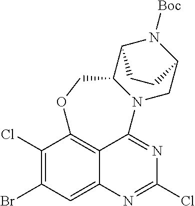 [SMILES: CC(C)(C)OC(=O)N1[C@@H]2CC[C@H]1[C@H]1COc3c(Cl)c(Br)cc4nc(Cl)nc(c34)N1C2]

Under nitrogen, a solution of tert-butyl (1 S,2S,5R)-2-(((7-bromo-2,6-dichloro-4-hydroxyquinazolin-5-yl)oxy)methyl)-3,8-diazabicyclo[3.2.1]octane-8-carboxylate (390 mg, 0.73 mmol), BOP-Cl (546 mg, 2.14 mmol) and DIPEA (1.49 g, 11.6 mmol) in dichloromethane (20 mL) was stirred at room temperature for 12 h. The reaction mixture was diluted with DCM and washed with water. The organic layer was dried over anhydrous sodium sulfate and concentrated under vacuum. The residue was purified by flash chromatography on silica gel (gradient: 0-5% methanol/DCM) to yield 270 mg (71% yield) of the title compound as a white solid. LC-MS: (ESI, m/z): [M+H]⁺=515. ¹H NMR (400 MHz, DMSO-d6, ppm) δ 7.75 (s, 1H), 4.82 (dd, J=13.5, 2.4 Hz, 1H), 4.74 (dd, J=13.4, 2.7 Hz, 1H), 4.57 (dd, J=13.3, 7.4 Hz, 1H), 4.39-4.26 (m, 2H), 4.11 (dt, J=7.3, 2.3 Hz, 1H), 3.14 (d, J=13.1 Hz, 1H), 1.88-1.67 (m, 4H), 1.45 (s, 9H).

### Intermediate 9: tert-Butyl (5aS,6S,9R)-2-bromo-3-chloro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate

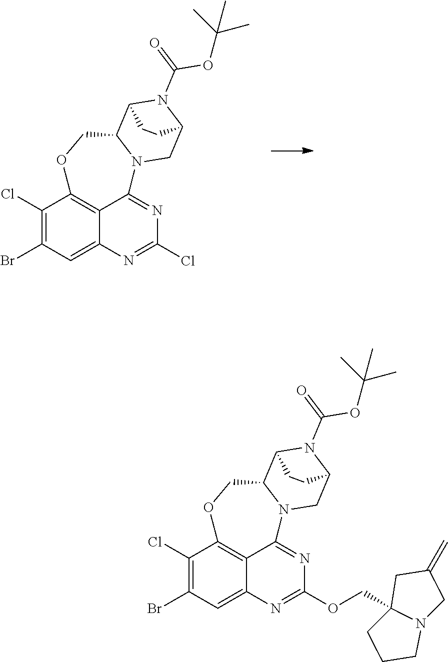 [SMILES: CC(C)(C)OC(=O)N1[C@@H]2CC[C@H]1[C@H]1COc3c(Cl)c(Br)cc4nc(Cl)nc(c34)N1C2 ; C=C1CN2CCC[C@@]2(COc2nc3c4c(c(Cl)c(Br)cc4n2)OC[C@@H]2[C@@H]4CC[C@H](CN32)N4C(=O)OC(C)(C)C)C1]

To an ice-cooled solution of (S)-(2-methylenetetrahydro-1H-pyrrolizin-7a(5H)-yl)methanol (92.5 mg, 0.600 mmol, Intermediate 15) in tetrahydrofuran (13 mL) was added NaH (100 mg, 2.50 mmol, 60% in mineral oil). The solution was warmed to room temperature for 30 minutes before recooling to 0° C. tert-Butyl (5aS,6S,9R)-2-bromo-3,13-dichloro-5a,6,7,8,9,10-hexahydro-5H-6,9-epiminoazepino[2′,1′:3,4][1,4]oxazepino[5,6,7-de]quinazoline-15-carboxylate (250 mg, 0.48 mmol, Intermediate 8) was added, and the reaction mixture was heated to 40° C. for 3 h. The reaction was cooled to 0° C., diluted with water and concentrated under vacuum. The residue was purified by pre-packed C18 column (solvent gradient: 0-100% ACN in water (0.05% NH₄HCO₃)) to yield 230 mg (75% yield) of the title compound as a yellow solid. LC-MS: (ESI, m/z): [M+H]⁺=632.

### Intermediate 10: 2,6-Dichloro-5,8-difluoro-7-(6-fluoro-1-methyl-1H-indazol-7-yl)quinazolin-4 (3H)-one

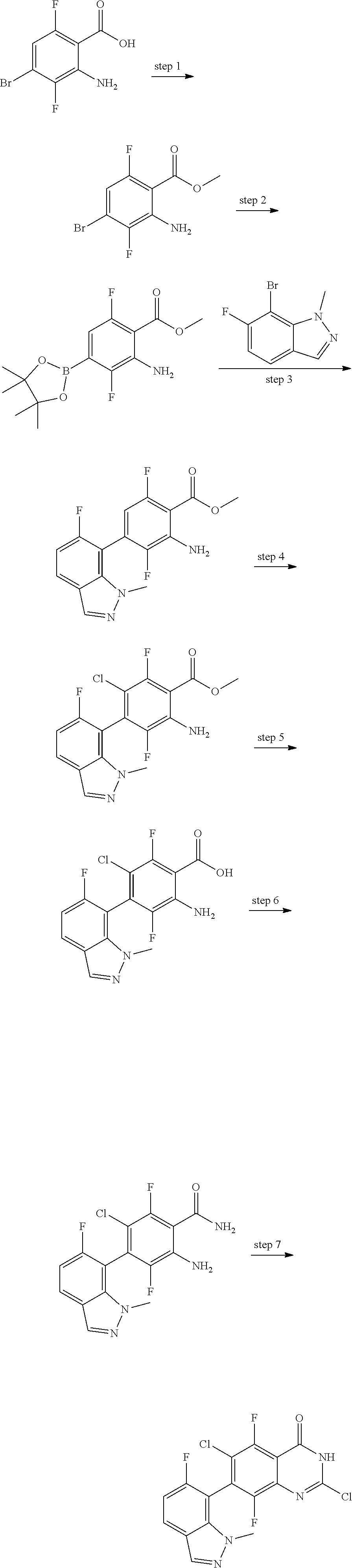

### Step 1: Methyl 2-mino-4-bromo-3,6-difluorobenzoate

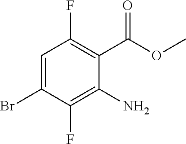

To an ice-cooled solution of 2-amino-4-bromo-3,6-difluoro-benzoic acid (2.72 g, 10.8 mmol, step 4 of intermediate 2) in ethyl acetate (13.5 mL) and methanol (13.5 mL) was added TMSCHN₂ (10.8 mL, 21.6 mmol, 2 mol/L in n-hexane). The reaction was warmed to 25° C. After 10 min, the mixture was concentrated under vacuum to afford the title compound (2.8 g, crude) as a yellow solid which was used without further purification. LC-MS: (ESI, m/z): [M+H]⁺=266.

### Step 2: Methyl 2-amino-3,6-difluoro-4-(4,4,5,5-tetramethyl-1,3,2-dioxaborolan-2-yl)benzoate

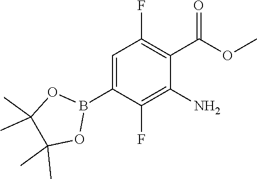

Under nitrogen, a solution of methyl 2-amino-4-bromo-3,6-difluoro-benzoate (2.87 g, 10.8 mmol), Pin₂B₂ (4.11 g, 16.2 mmol), Pd(dppf)Cl₂ (788 mg, 1.08 mmol) and KOAc (3.17 g, 32.3 mmol) in 1,4-dioxane (72 mL) was stirred for 1 h at 100° C. The solid was filtered, and the filtrate was concentrated under reduced pressure. The residue was purified by flash chromatography on silica gel (gradient: 0-20% ethyl acetate/petroleum ether) to afford crude product (containing 20% Pin₂B₂). The crude was stirred in 10 mL petroleum ether for 10 min, and the solid was collected. The process was repeated 3× to yield 2.0 g (59% yield) of the title compound as a white solid. LC-MS: (ESI, m/z): [M+H]⁺=314.

### Step 3: Methyl 2-amino-3,6-difluoro-4-(6-fluoro-1-methyl-1H-indazol-7-yl)benzoate

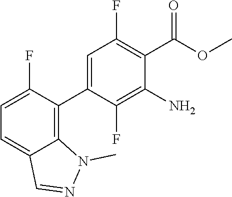 [SMILES: COC(=O)c1c(F)cc(-c2c(F)ccc3cnn(C)c23)c(F)c1N]

Under nitrogen, a solution of methyl 2-amino-3,6-difluoro-4-(4,4,5,5-tetramethyl-1,3,2-dioxaborolan-2-yl)benzoate (1.64 g, 5.24 mmol), 7-bromo-6-fluoro-1-methyl-indazole (1.44 g, 6.3 mmol), Pd(PPh₃)₂Cl₂ (368 mg, 0.520 mmol) and KF (913 mg, 15.7 mmol) in acetonitrile (20 mL) and water (4 mL) was stirred for 1 h at 80° C. The solid was filtered, and the filtrate was concentrated under reduced pressure. The residue was purified by flash chromatography on silica gel (gradient: 0-7% MeCOH/DCM) to yield 1.25 g (71% yield) of the title compound as a yellow solid. LC-MS: (ESI, m/z): [M+H]⁺=336.

### Step 4: Methyl 2-amino-5-chloro-3,6-difluoro-4-(6-fluoro-1-methyl-1H-indazol-7-yl)benzoate

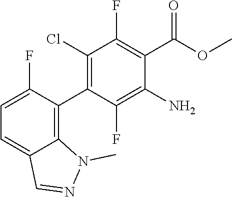 [SMILES: COC(=O)c1c(N)c(F)c(-c2c(F)ccc3cnn(C)c23)c(Cl)c1F]

Under nitrogen, a solution of methyl 2-amino-3,6-difluoro-4-(6-fluoro-1-methyl-indazol-7-yl)benzoate (1.21 g, 3.60 mmol), NCS (574 mg, 4.32 mmol) in N,N-dimethylformamide (12 mL) was stirred at 80° C. for 1 h. The reaction was quenched with saturated aqueous Na₂S₂O₃ solution and diluted with ethyl acetate (100 mL). The resulting solution was washed with water followed by brine. The organic layer was dried over anhydrous sodium sulfate and concentrated under vacuum. The residue was purified by flash chromatography on silica gel (gradient: 0-15% ethyl acetate/petroleum ether) to yield 1.1 g (83% yield) of the title compound as a yellow solid. LC-MS: (ESI, m/z): [M+H]⁺=370.

### Step 5: 2-Amino-5-chloro-3,6-difluoro-4-(6-fluoro-1-methyl-1H-indazol-7-yl)benzoic acid

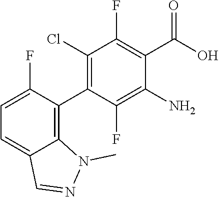 [SMILES: Cn1ncc2ccc(F)c(-c3c(F)c(N)c(C(=O)O)c(F)c3Cl)c21]

To a solution of methyl 2-amino-5-chloro-3,6-difluoro-4-(6-fluoro-1-methyl-indazol-7-yl)benzoate (1.10 g, 3.08 mmol) in tetrahydrofuran (12 mL) was added LiOH (212 mg, 9.23 mmol) in water (4 mL) at room temperature. After 18 h, the reaction mixture was acidified to pH to 5-6 with 1M aqueous HCl and extracted with ethyl acetate (3×). The combined organic layers were dried over anhydrous/sodium sulfate and concentrated under vacuum to afford 1.0 g (crude) of title compound. LC-MS: (ESI, m/z): [M+H]⁺=356. The crude was used without further purification.

### Step 6: 2-Amino-5-chloro-3,6-difluoro-4-(6-fluoro-1-methyl-1H-indazol-7-yl)benzamide

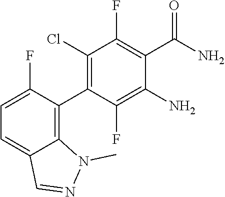 [SMILES: Cn1ncc2ccc(F)c(-c3c(F)c(N)c(C(N)=O)c(F)c3Cl)c21]

A solution of 2-amino-5-chloro-3,6-difluoro-4-(6-fluoro-1-methyl-1H-indazol-7-yl)benzamide (1 g, crude), NH₄Cl (830 mg, 15.4 mmol), HATU (1.75 g, 4.61 mmol) and DIPEA (2.78 g, 21.53 mmol) in N,N-dimethylformamide (15 mL) was stirred for 3 h at room temperature. The solution was diluted with EtOAc (100 mL) and washed with saturated aqueous NH₄Cl solution. The organic layer was dried over anhydrous sodium sulfate and concentrated under vacuum. The residue was purified by flash chromatography on silica gel (gradient: 0-4% MeCOH/DCM) to yield 950 mg (87% yield) of the title compound as a white solid. LC-MS: (ESI, m/z): [M+H]⁺=355.

### Step 7: 2,6-Dichloro-5,8-difluoro-7-(6-fluoro-1-methyl-1H-indazol-7-yl)quinazolin-4 (3H)-one

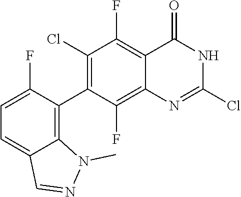 [SMILES: Cn1ncc2ccc(F)c(-c3c(Cl)c(F)c4c(=O)[nH]c(Cl)nc4c3F)c21]

A solution of 2-amino-5-chloro-3,6-difluoro-4-(6-fluoro-1-methyl-indazol-7-yl)benzamide (1.01 g, 2.85 mmol) and thiophosgene (0.65 mL, 8.55 mmol) in 1,4-dioxane (20 mL) was stirred at room temperature for 1 h and then heated at reflux for 1 h. The reaction mixture was concentrated under vacuum. The residue was purified by flash chromatography on silica gel (gradient: 0%-5% MeCOH/DCM) to afford the title compound (1.27 g, crude), 76% purity) as a brown solid. LC-MS: (ESI, m/z): [M+H]⁺=399. ¹H NMR (400 MHz, DMSO-d₆) δ 8.22 (s, 1H), 8.04 (dd, J=8.8, 5.2 Hz, 1H), 7.30-7.24 (m, 1H), 3.56 (s, 3H).

### Intermediate 11: (3-Methylene-1-azabicyclo[3.2.0]heptan-5-yl)methanol

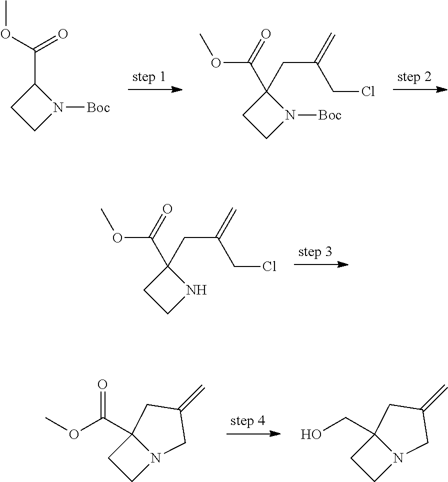

### Step 1: 1-(tert-Butyl) 2-methyl 2-(2-(chloromethyl)allyl)azetidine-1,2-dicarboxylate

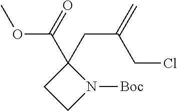 [SMILES: C=C(CCl)CC1(C(=O)OC)CCN1C(=O)OC(C)(C)C]

Under nitrogen, to a solution of 1-tert-butyl 2-methyl azetidine-1,2-dicarboxylate (0.300 g, 1.39 mmol) in tetrahydrofuran (7 mL) was added LiHMDS (2.8 mL, 2.8 mmol, 1M in THF) at −20° C. After 0.5 h, 3-Chloro-2-Chloromethyl-1-Propene (349 mg, 2.79 mmol) was added at −20° C. The resulting solution was warmed to room temperature for 1 h. The reaction was quenched with saturated aqueous NH₄Cl solution, diluted with water and extracted with EtOAc (3×). The combined organic layers were dried over anhydrous sodium sulfate and concentrated under vacuum. The residue was purified by flash chromatography on silica gel eluting with (EtOAc/Petroleum ether=0-30%) to afford the title compound (0.170 g, 40. % yield) as a yellow oil. LC-MS: (ESI, m/z): [M+H]⁺=304. ¹H NMR (300 MHz, Chloroform-d) δ 5.42 (d, J=1.3 Hz, 1H), 5.10 (d, J=1.2 Hz, 1H), 4.26-4.02 (m, 2H), 4.01-3.94 (m, 1H), 3.78 (d, J=5.4 Hz, 3H), 3.60 (s, 1H), 3.01 (dd, J=14.9, 1.1 Hz, 1H), 2.64 (d, J=14.8 Hz, 1H), 2.42-2.33 (m, 1H), 2.11 (d, J=15.6 Hz, 1H), 1.43 (s, 9H).

### Step 2: Methyl 2-(2-(chloromethyl)allyl)azetidine-2-carboxylate

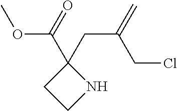

A solution of 1-(tert-butyl) 2-methyl 2-(2-(chloromethyl)allyl)azetidine-1,2-dicarboxylate (170 mg, 0.560 mmol) in 2,2,2-trifluoroacetic acid (0.4 mL) and dichloromethane (1.6 mL) was stirred at room temperature for 0.5 h. The resulting mixture was concentrated under vacuum to afford the title compound as a TFA salt (110 mg, crude yellow solid). LC-MS: (ESI, m/z): [M+H]⁺=204. The crude product was used without further purification.

### Step 3: Methyl 3-methylene-1-azabicyclo[3.2.0]heptane-5-carboxylate

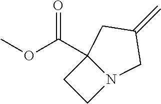

A mixture of methyl 2-[2-(chloromethyl)allyl]azetidine-2-carboxylate TFA salt (110 mg, 0.540 mmol) and K₂CO₃ (225 mg, 1.63 mmol) in acetonitrile (4 mL) was stirred at room temperature for 3 h. The solid was filtered, and the filtrate was concentrated under reduced pressure. The residue was purified by flash chromatography on silica gel eluting with (MeOH/DCM=0-10%) to afford the title compound (48 mg, 20% yield over 3 steps) as a light yellow oil. LC-MS: (ESI, m/z): [M+H]⁺=168. The crude product was used without further purification. ¹H NMR (300 MHz, Chloroform-d) δ 5.22-5.14 (m, 1H), 5.13-5.04 (m, 1H), 3.78 (d, J=2.6 Hz, 3H), 3.62-3.44 (m, 2H), 3.22-3.10 (m, 1H), 3.02-2.84 (m, 2H), 2.84-2.73 (m, 2H), 2.25-2.14 (m, 1H).

### Step 4: (3-Methylene-1-azabicyclo[3.2.0]heptan-5-yl)methanol

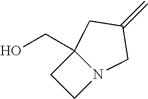

To an ice-cooled solution of methyl 3-methylene-1-azabicyclo[3.2.0]heptane-5-carboxylate (40.1 mg, 0.240 mmol) in tetrahydrofuran (1 mL) was added LiAlH₄ (0.5 mL, 0.5 mmol, 1M in THF). After 0.5 h, the reaction was quenched with Na₂SO₄·10H₂O and diluted with ether (10 mL). The solid was filtered, and the filtrate was concentrated under reduced pressure to afford the title compound (21.1 mg, crude) as a colorless oil. LC-MS: (ESI, m/z): [M+H]⁺=140. The crude product was used without further purification.

### Intermediate 12: (S)-(1-(2,2-Difluoroethyl)azetidin-2-yl)methanol

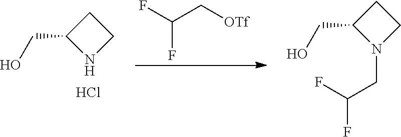

To an ice-cooled solution of K₂CO₃ (0.500 g, 3.62 mmol) in acetonitrile (5 mL) and (S)-azetidin-2-ylmethanol hydrochloride (200 mg, 1.63 mmol) was added 2,2-difluoroethyl trifluoromethanesulfonate (0.360 g, 1.68 mmol). The reaction mixture warmed to room temperature. After 24 h, the reaction was diluted with water (30 mL), and the resulting mixture was extracted with EtOAc. The combined organic layers were dried over anhydrous sodium sulfate and concentrated under vacuum. The residue was purified by flash chromatography on silica gel (gradient: 0-60% ethyl acetate/petroleum ether) to yield 0.090 g (36% yield) of the title compound as a colorless oil. LC-MS: (ESI, m/z): [M+H]⁺=152.

### Intermediate 13: (2-Methylenetetrahydro-1H-pyrrolizin-7a(5H)-yl)methanol

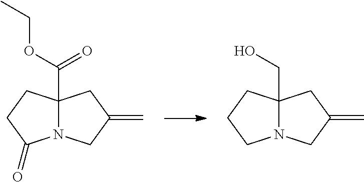

To a solution of ethyl 2-methylene-5-oxotetrahydro-1H-pyrrolizine-7a(5H)-carboxylate (0.20 g, 0.96 mmol) in tetrahydrofuran (20 mL) under nitrogen was added LiAlH₄ (2.87 mL, 1 M in THF) at −40° C. The resulting solution was warmed to room temperature for 3 h. The reaction mixture was quenched with Na₂SO₄·10H₂O. The solid was filtered and rinsed with EtOAc. The filtrate was concentrated under reduced pressure to afford the crude product (150 mg). The crude product was used without purification. LC-MS: (ESI, m/z): [M+H]⁺=154.2.

### Intermediate 14 and 15: (R)-(2-Methylenetetrahydro-1H-pyrrolizin-7a(5H)-yl)methanol and (S)-(2-Methylenetetrahydro-1H-pyrrolizin-7a(5H)-yl)methanol

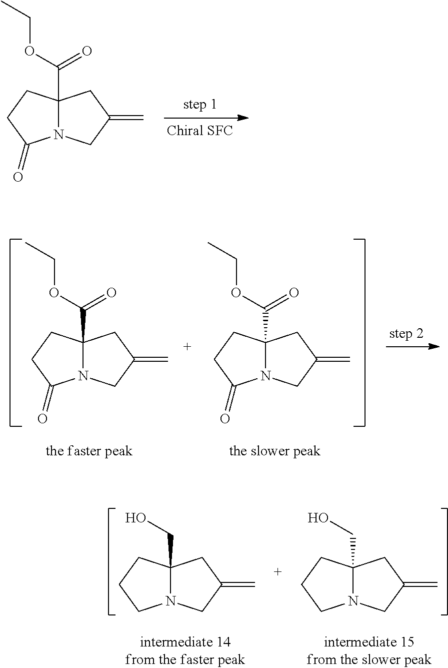

### Step 1: Ethyl (R)-2-methylene-5-oxotetrahydro-1H-pyrrolizine-7a(5H)-carboxylate & Ethyl (S)-2-methylene-5-oxotetrahydro-1H-pyrrolizine-7a(5H)-carboxylate

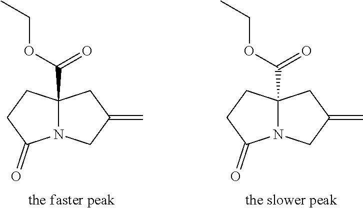

Ethyl 2-methylene-5-oxotetrahydro-1H-pyrrolizine-7a(5H)-carboxylate (19.9 g) was separated by Chiral SFC (Column: CHIRALPAK IH, 50*250 mm; Mobile Phase A: CO2, Mobile Phase B: EtOH; Flow rate: 150 mL/min; Gradient:26% B; 220 nm; RT1:4.8; RT2:6.43; Injection Volumn:1.8 ml; Number Of Runs: 122) to afford ethyl (R)-2-methylene-5-oxotetrahydro-1H-pyrrolizine-7a(5H)-carboxylate (7.61 g, the faster peak) and ethyl (S)-2-methylene-5-oxotetrahydro-1H-pyrrolizine-7a(5H)-carboxylate (7.29 g, the slower peak). LC-MS: (ESI, m/z): [M+H]⁺=210. ¹H NMR (400 MHz, Chloroform-d) δ 5.12-5.00 (m, 2H), 4.32-4.28 (m, 1H), 4.21 (q, J=7.1 Hz, 2H), 3.73 (d, J=15.7 Hz, 1H), 3.06 (d, J=15.7 Hz, 1H), 2.85-2.72 (m, 1H), 2.66-2.57 (m, 1H), 2.53-2.41 (m, 2H), 2.19-2.08 (m, 1H), 1.28 (t, J=7.1 Hz, 3H). The HNMR of two isomers are same.

### Step 2: (S)-(2-Methylenetetrahydro-1H-pyrrolizin-7a(5H)-yl)methanol

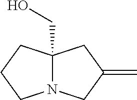

To an ice-cooled solution of ethyl (S)-2-methylene-5-oxotetrahydro-1H-pyrrolizine-7a(5H)-carboxylate (110 mg, 0.51 mmol) in tetrahydrofuran (5 mL) under nitrogen was added LiAlH₄ (1.1 mL, 1M in THF). The mixture was heated for 0.5 h at 70° C. The mixture was cooled to room temperature and quenched with Na₂SO₄·10H₂O and filtered. The solvent was removed by blowing nitrogen (low boiling point for the product) to afford the title compound (62.8 mg, crude). LC-MS: (ESI, m/z): [M+H]⁺=154. The crude was used without further purification.

Analogous to method described in step 2, the enantiomer (R)-(2-methylenetetrahydro-1H-pyrrolizin-7a(5H)-yl)methanol (intermediate 14) was synthesized.

### Intermediate 16: (S)-(2,2-Difluorotetrahydro-1H-pyrrolizin-7a(5H)-yl)methanol

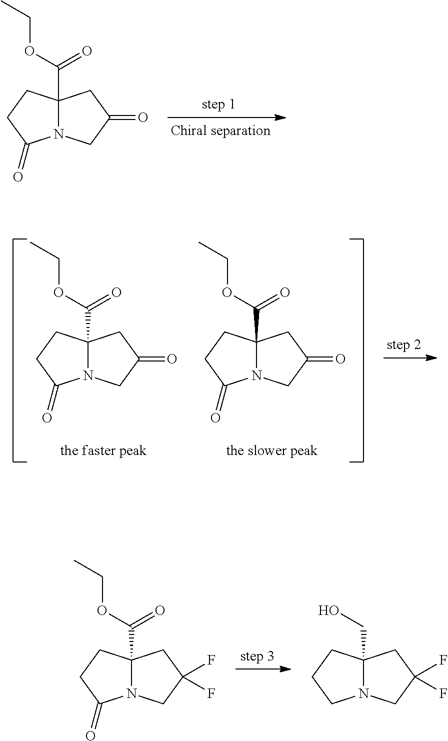

### Step 1: Ethyl (S)-2,5-dioxotetrahydro-1H-pyrrolizine-7a(5H)-carboxylate and Ethyl (R)-2,5-dioxotetrahydro-1H-pyrrolizine-7a(5H)-carboxylate

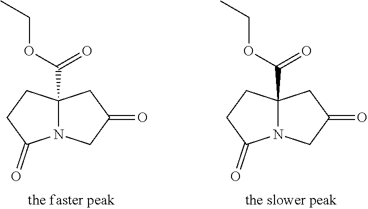

Ethyl 2,5-dioxotetrahydro-1H-pyrrolizine-7a(5H)-carboxylate (20.0 g, 94.6 mmol) was separated by (Column: AD 2.12*25 cm, 5 μm; Mobile Phase A: CO₂, Mobile Phase B: EtOH:ACN=1:1; Flow rate:200 mL/min; Gradient:50% B; 220 nm; RT1: 2.44; RT2: 3.58; Injection Volumn:10 ml; Number Of Runs:15) to afford ethyl (S)-2,5-dioxotetrahydro-1H-pyrrolizine-7a(5H)-carboxylate (8.21 g, the faster peak) as a yellow oil and ethyl (R)-2,5-dioxotetrahydro-1H-pyrrolizine-7a(5H)-carboxylate (7.92 g, the slower peak) as a yellow oil. LC-MS: (ESI, m/z): [M+H]⁺=212. ¹H NMR (400 MHz, Chloroform-d) δ 4.22 (q, J=7.2 Hz, 2H), 4.14-4.05 (m, 1H), 3.54 (d, J=18.6 Hz, 1H), 3.02-2.91 (m, 2H), 2.87-2.72 (m, 1H), 2.60-2.38 (m, 2H), 2.23-2.11 (m, 1H), 1.28 (t, J=7.1 Hz, 3H).

### Step 2: Ethyl (S)-2,2-difluoro-5-oxotetrahydro-1H-pyrrolizine-7a(5H)-carboxylate

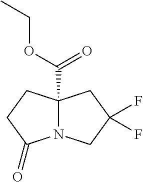 [SMILES: CCOC(=O)[C@@]12CCC(=O)N1CC(F)(F)C2]

To an ice-cooled solution of ethyl (S)-2,5-dioxotetrahydro-1H-pyrrolizine-7a(5H)-carboxylate (0.20 g, 0.95 mmol) in dichloromethane (30 mL) under nitrogen was added DAST (378 mg, 2.35 mmol). The mixture was warmed to room temperature. After 3 h, the reaction mixture was concentrated under vacuum. The residue was purified by flash chromatography on silica gel (gradient: 0-2% MeCOH/DCM) to afford 98.3 mg (44.4% yield) of the title compound as awhite solid. LC-MS: (ESI, m/z): [M+H]⁺=234. ¹H NMR (400 MHz, Chloroform-d) δ 4.28 (q, J=7.1 Hz, 2H), 4.20-4.07 (m, 1H), 3.54-3.39 (m, 1H), 3.08-2.5 (m, 1H), 2.82-2.60 (m, 2H), 2.51-2.12 (m, 3H), 1.32 (t, J=7.1 Hz, 3H).

### Step 3: (S)-(2,2-Difluorotetrahydro-1H-pyrrolizin-7a(5H)-yl)methanol

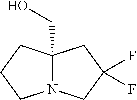 [SMILES: OC[C@@]12CCCN1CC(F)(F)C2]

To an ice-cooled solution of ethyl (S)-2,2-difluoro-5-oxotetrahydro-1H-pyrrolizine-7a(5H)-carboxylate (95.3 mg, 0.410 mmol) in tetrahydrofuran (10 mL) under nitrogen was added LiAlH₄ (2.0 mL, 1M in THF). The mixture was heated to 70° C. for 3 h. The mixture was quenched with Na₂SO₄·10H₂O and filtered. The solvent was removed by gently blowing nitrogen (low boiling point) to afford 70.5 mg (crude). LC-MS: (ESI, m/z): [M+H]⁺=178. The crude product was used without further purification.

### Intermediate 17: (R)-(2,2-difluorotetrahydro-1H-pyrrolizin-7a(5H)-yl)methanol

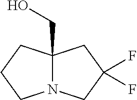 [SMILES: OC[C@]12CCCN1CC(F)(F)C2]

Analogous to method described in Intermediate 16, the title compound was prepared from ethyl (R)-2,5-dioxotetrahydro-1H-pyrrolizine-7a(5H)-carboxylate. LC-MS: (ESI, m/z): [M+H]⁺=178. The crude was used for the next step without further purification.

### Intermediate 18: (Hexahydro-1H-pyrrolo[2,1-c][1,4]oxazin-6-yl)methanol (mixture of trans)

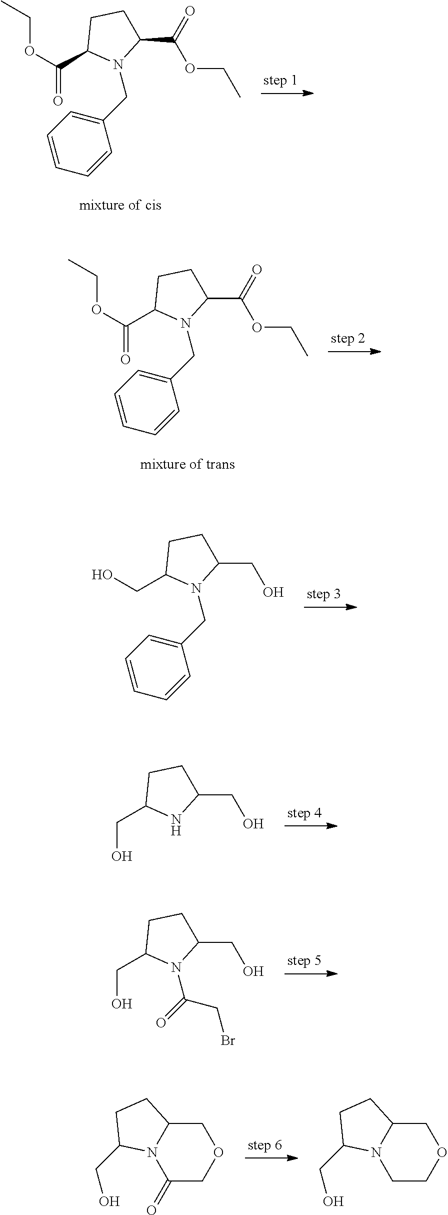

### Step 1: Diethyl 1-benzylpyrrolidine-2,5-dicarboxylate (mixture of trans)

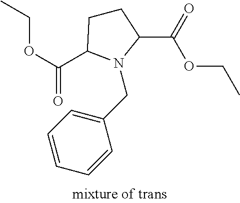 [SMILES: CCOC(=O)C1CCC(C(=O)OCC)N1Cc1ccccc1]

To a solution of diethyl cis-1-benzylpyrrolidine-2,5-dicarboxylate (8.60 g, 28.2 mmol) in tetrahydrofuran (120 mL) was added LiHMDS (43.4 mL, 56.4 mmol) at −35° C. After 1 h, the reaction was quenched with saturated aqueous NH₄Cl solution and extracted with EtOAc. The combined organic layers were washed with brine, dried over anhydrous sodium sulfate, and concentrated under vacuum. The residue was purified by flash chromatography on silica gel (gradient: 0-15% ethyl acetate/petroleum ether) to afford 1.27 g (14% yield) of the title compound as a light yellow oil. The cis-isomer was recovered (6.3 g). LC-MS: (ESI, m/z): [M+H]⁺=306.2. ¹H NMR (300 MHz, DMSO-d₆, ppm) δ 7.32-7.20 (m, 5H), 4.13-3.87 (m, 6H), 3.75-3.60 (m, 3H), 2.25-2.11 (m, 2H), 1.91-1.75 (m, 2H), 1.25-1.06 (m, 3H).

### Step 2: (1-Benzylpyrrolidine-2,5-diyl)dimethanol (mixture of trans)

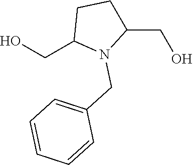 [SMILES: OCC1CCC(CO)N1Cc1ccccc1]

To an ice-cooled solution of diethyl trans-1-benzylpyrrolidine-2,5-dicarboxylate (2.30 g, 7.53 mmol) in tetrahydrofuran (30 mL) under nitrogen was added LiAlH₄ (716 mg, 18.8 mmol) in several portions. The reaction was warmed to room temperature. After 2 h, the mixture was quenched with Na₂SO₄·10H₂O. The solid was filtered, and the filtrate was concentrated under vacuum. The residue was purified by flash chromatography on silica gel (gradient: 0-10% MeCOH/DCM) to yield 1.65 g (99% yield) of the title compound as a yellow oil. LC-MS: (ESI, m/z): [M+H]⁺=222.1. ¹H NMR (400 MHz, DMSO-d₆, ppm) δ 7.38-7.15 (m, 5H), 4.34 (s, 2H), 3.95-3.82 (m, 2H), 3.46-3.36 (m, 2H), 3.28-3.20 (m, 2H), 3.01-2.90 (m, 2H), 1.90-1.75 (m, 2H), 1.71-1.58 (m, 2H).

### Step 3: Pyrrolidine-2,5-diyldimethanol (mixture of trans)

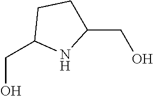

Under hydrogen (1 atm), a solution of (1-benzylpyrrolidine-2,5-diyl)dimethanol (0.60 g, 2.7 mmol) and Pd/C (180 mg, 10% w/w) in methyl alcohol (10 mL) was stirred at room temperature for 2 h. The catalyst was filtered, and the filtrate was concentrated to afford 405 mg (crude) of the title compound as a light yellow oil. LC-MS: (ESI, m/z): [M+H]⁺=132.1. The crude product was used without further purification.

### Step 4: 1-(2,5-Bis(hydroxymethyl)pyrrolidin-1-yl)-2-bromoethan-1-one (mixture of trans)

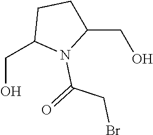 [SMILES: O=C(CBr)N1C(CO)CCC1CO]

To an ice-cooled solution of pyrrolidine-2,5-diyldimethanol (355 mg, 2.71 mmol) and N-methyl morpholine (410 mg, 4.06 mmol) in tetrahydrofuran (10 mL) was added 2-bromoacetyl bromide (539 mg, 2.67 mmol). After 1 h, the reaction was concentrated under vacuum. The residue was purified by flash chromatography on silica gel (gradient: 0-10% MeCOH/DCM) to yield 130 mg (19% yield) of the title compound as a yellow oil. LC-MS: (ESI, m/z): [M+H]⁺=252.1. ¹H NMR (400 MHz, DMSO-d₆, ppm) δ 5.03 (s, 1H), 4.71 (s, 1H), 4.24 (d, J=11.4 Hz, 1H), 4.09-3.99 (m, 1H), 3.92-3.87 (m, 2H), 3.69 (s, 2H), 3.17 (s, 2H), 2.04-1.72 (m, 4H).

### Step 5: 6-(Hydroxymethyl)tetrahydro-1H-pyrrolo[2,1-c][1,4]oxazin-4 (3H)-one (mixture of trans)

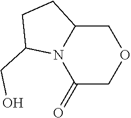 [SMILES: O=C1COCC2CCC(CO)N12]

To an ice-cooled suspension of NaH (65.0 mg, 1.63 mmol, 60% in mineral oil) in tetrahydrofuran (5 mL) was added 1-(2,5-bis(hydroxymethyl)pyrrolidin-1-yl)-2-bromoethan-1-one (130 mg, 0.517 mmol) in 1 mL THF. After 1 h, the resulting solution was warmed to room temperature for 3 h. The reaction was diluted with water and concentrated under vacuum. The residue was purified by flash chromatography on silica gel (gradient: 0-10% MeCOH/DCM) to yield the title compound as an oil (37.0 mg, 42% yield). LC-MS: (ESI, m/z): [M+H]⁺=172.2. ¹H NMR (400 MHz, DMSO-d₆, ppm) δ 4.88-4.82 (m, 1H), 4.14-3.97 (m, 3H), 3.84 (d, J=16 Hz, 1H), 3.71-3.61 (m, 1H), 3.59-3.48 (m, 2H), 3.23-3.15 (m, 1H), 2.03-1.87 (m, 2H), 1.82-1.66 (m, 1H), 1.39-1.19 (m, 1H).

### Step 6: (Hexahydro-1H-pyrrolo[2,1-c][1,4]oxazin-6-yl)methanol (mixture of trans)

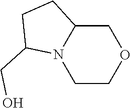 [SMILES: OCC1CCC2COCCN12]

To an ice-cooled suspension of LiAlH₄ (27.3 mg, 0.720 mmol) in tetrahydrofuran (5 mL) under nitrogen was added 6-(hydroxymethyl)tetrahydro-1H-pyrrolo[2,1-c][1,4]oxazin-4 (3H)-one (60.0 mg, 0.350 mmol) in 0.5 mL THF. The resulting solution warmed to 60° C. for 2 h. The reaction was cooled to room temperature and quenched with Na₂SO₄·10H₂O. The solid was filtered, and the filtrate was concentrated to yield the title compound as a light yellow oil (60 mg, crude). LC-MS: (ESI, m/z): [M+H]⁺=158.1. The crude product was used without further purification.

### Intermediate 19: (S)-(2-(Difluoromethylene)tetrahydro-1H-pyrrolizin-7a(5H)-yl)methanol

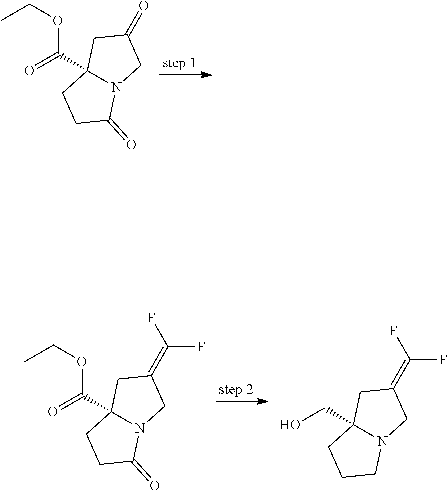 [SMILES: CCOC(=O)[C@@]12CCC(=O)N1CC(=O)C2 ; CCOC(=O)[C@@]12CCC(=O)N1CC(=C(F)F)C2 ; OC[C@@]12CCCN1CC(=C(F)F)C2]

### Step 1: Ethyl (S)-2-(difluoromethylene)-5-oxotetrahydro-1H-pyrrolizine-7a(5H)-carboxylate

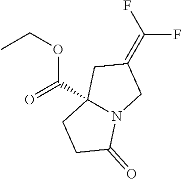 [SMILES: CCOC(=O)[C@@]12CCC(=O)N1CC(=C(F)F)C2]

To a solution of ethyl (S)-2,5-dioxotetrahydro-1H-pyrrolizine-7a(5H)-carboxylate (0.20 g, 0.95 mmol, Intermediate 16, step 1, the faster peak) and 2-((difluoromethyl)sulfonyl)pyridine (237 mg, 1.23 mmol) in N,N-dimethylformamide (6 mL) under nitrogen was added t-BuOK (191 mg, 1.71 mmol) in DMF (2 mL) slowly at −50° C. The reaction was warmed to −40° C. for 1 h, and the reaction was quenched with saturated aqueous ammonium chloride solution (2 mL) and 3 M HCl (2 mL). The mixture was stirred for 16 h at room temperature. The mixture was diluted with water (20 mL) and extracted with EtOAc. The combined organics were dried over anhydrous sodium sulfate and concentrated under vacuum. The residue was purified by flash chromatography on silica gel (gradient: 0-40% EtOAc/DCM) to yield the title compound as a yellow oil (28.0 mg, 12% yield). LC-MS: (ESI, m/z): [M+H]⁺=246.1.

### Step 2: (S)-(2-(Difluoromethylene)tetrahydro-1H-pyrrolizin-7a(5H)-yl)methanol

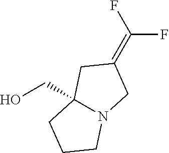 [SMILES: OC[C@@]12CCCN1CC(=C(F)F)C2]

To an ice-cooled solution of ethyl (S)-2-(difluoromethylene)-5-oxotetrahydro-1H-pyrrolizine-7a(5H)-carboxylate (28.0 mg, 0.110 mmol) in tetrahydrofuran (2 mL) under nitrogen was added DIBAL-H (1.12 mL, 1.12 mmol). The reaction mixture was warmed to room temperature. After 1 h, the solution was quenched with Na₂SO₄·10H₂O. The solid was filtered, and the filtrate was concentrated under reduced pressure. LC-MS: (ESI, m/z): [M+H]⁺=190.1. The crude product was used further purification.

### Intermediate 20: (R)-(2-(Difluoromethylene)tetrahydro-1H-pyrrolizin-7a(5H)-yl)methanol

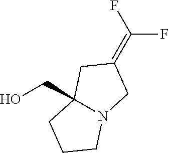 [SMILES: OC[C@]12CCCN1CC(=C(F)F)C2]

Analogous to method described in Intermediate 19, the title compound was prepared from (R)-2,5-dioxotetrahydro-1H-pyrrolizine-7a(5H)-carboxylate (Intermediate 16, step 1, the slower peak) and 2-((difluoromethyl)sulfonyl)pyridine. LC-MS: (ESI, m/z): [M+H]⁺=190.1. The crude was used without further purification.

### Intermediate 21: (S)-(2-(Fluoromethylene)tetrahydro-1H-pyrrolizin-7a(5H)-yl)methanol (mixture of Z/E)

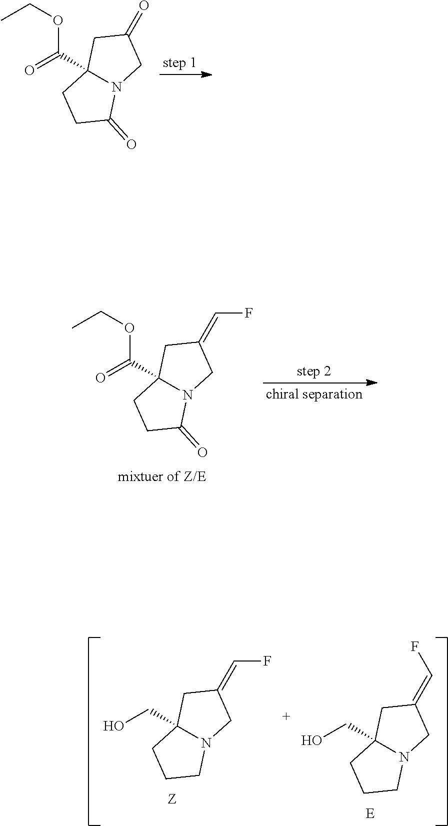

### Step 1: Ethyl (S)-2-(fluoromethylene)-5-oxotetrahydro-1H-pyrrolizine-7a(5H)-carboxylate

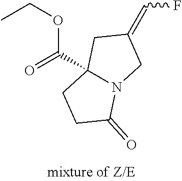 [SMILES: CCOC(=O)[C@@]12CCC(=O)N1CC(=CF)C2]

To a solution of 2-((fluoromethyl)sulfonyl)pyridine (177 mg, 1.01 mmol) in tetrahydrofuran (10 mL) under nitrogen was added KHMDS (1.2 mL, 1.20 mmol) at −78° C. After 30 min, ethyl (S)-2,5-dioxotetrahydro-1H-pyrrolizine-7a(5H)-carboxylate (0.20 mg, 0.95 mmol, Intermediate 16, step 1, the faster peak) in THF (5 mL) was added slowly at −78° C. After 3 h, the reaction system was warmed to room temperature for 1 h. The reaction was quenched with aqueous saturated aqueous ammonium chloride solution (1 mL), followed by 3 M HCl (2 mL). The mixture was stirred for 1 h at room temperature, diluted with water, and extracted with EtOAc. The combined organics were dried over anhydrous sodium sulfate and concentrated under vacuum. The residue was purified by flash chromatography on silica gel (gradient: 0-30% ethyl acetate/petroleum ether) to yield the title compound as a yellow oil (65 mg, 30% yield). LC-MS: (ES, m/z): [M+1]⁺=228.1. ¹H NMR (400 MHz, Chloroform-d₁, ppm) δ 6.75-6.65 (m, 1H), 6.55-6.44 (m, 1H), 4.45-4.32 (m, 2H), 4.27-4.20 (m, 4H), 3.90 (d, J=16 Hz, 1H), 3.73 (d, J=16 Hz, 1H), 3.32 (d, J=16 Hz, 1H), 3.04 (d, J=16 Hz, 1H), 2.89-2.75 (m, 2H), 2.72-2.56 (m, 2H), 2.53-2.35 (m, 4H), 2.25-2.08 (m, 2H), 1.33-1.28 (m, 6H).

### Step 2: (S,Z)-(2-(Fluoromethylene)tetrahydro-1H-pyrrolizin-7a(5H)-yl)methanol and (S,E)-(2-(fluoromethylene)tetrahydro-1H-pyrrolizin-7a(5H)-yl)methanol

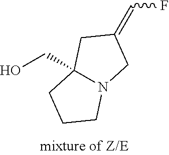 [SMILES: OC[C@@]12CCCN1CC(=CF)C2]

To an ice-cooled solution of ethyl (S,Z/E)-2-(fluoromethylene)-5-oxotetrahydro-1H-pyrrolizine-7a(5H)-carboxylate (0.060 g, 0.26 mmol) in tetrahydrofuran (5 mL) was added DIBAL-H (2.64 mL, 2.64 mmol). The resulting solution was warmed to room temperature for 1 h. The reaction was quenched with Na₂SO₄·10H₂O. The solid was filtered, and the filtrate was concentrated under reduced pressure. The residue was purified by flash chromatography on silica gel (gradient: 0-20% MeCOH/DCM (0.1% Et₃N)) to yield the title compound as a yellow oil (26 mg, 57% yield) of. LC-MS: (ESI, m/z): [M+H]⁺=172.1. ¹H NMR (300 MHz, Chloroform-d₁, ppm) δ 6.79-6.38 (m, 2H), 3.92-3.70 (m, 2H), 3.61-3.20 (m, 8H), 2.79-2.44 (m, 5H), 2.36-2.26 (m, 1H), 2.14-1.66 (m, 8H). (diploid H due to it is a mixture of Z/E).

On a larger scale, 2.20 g (Z isomer, first fraction) and 2.70 g (E isomer, second fraction) were obtained. from 9.7 g ethyl (S,Z/E)-2-(fluoromethylene)-5-oxotetrahydro-1H-pyrrolizine-7a(5H)-carboxylate following the same procedure and purification method described above.

Z isomer: LC-MS: (ESI, m/z): [M+H]⁺=172. ¹H NMR (300 MHz, Chloroform-d₁, ppm) δ 6.64-6.29 (m, 1H), 3.79-3.65 (m, 1H), 3.48-3.37 (m, 1H), 3.35-3.23 (m, 2H), 3.14-3.03 (m, 1H), 2.69-2.58 (m, 1H), 2.46-2.35 (m, 1H), 2.30-2.17 (m, 1H), 2.03-1.59 (m, 4H).

E isomer LC-MS: (ESI, m/z): [M+H]⁺=172. ¹H NMR (300 MHz, Chloroform-d₁, ppm) δ 6.76-6.42 (m, 1H), 3.75-3.65 (m, 1H), 3.48-3.34 (m, 2H), 3.33-3.18 (m, 2H), 2.73-2.40 (m, 3H), 2.09-1.65 (m, 4H).

### Intermediate 22: 6-Bromo-5-chloro-N,N-bis(4-methoxybenzyl)-4-methylpyridin-2-amine

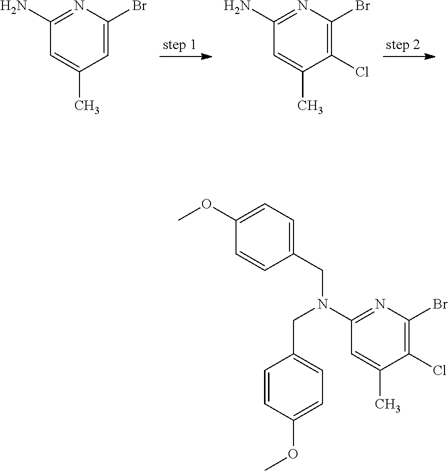

### Step 1: 6-Bromo-5-chloro-4-methylpyridin-2-amine

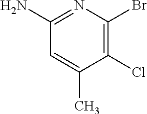

A solution of 6-bromo-4-methylpyridin-2-amine (500 mg, 2.67 mmol) and NCS (360 mg, 2.69 mmol) in DMF (5 mL) was stirred at 60° C. for 1 hour. The resulting solution was cooled to room temperature, diluted with EtOAc and washed with water (3×). The organic layer was dried over anhydrous Na₂SO₄ and concentrated under vacuum. The residue was purified by flash chromatography on silica gel (gradient: 0%-30% ethyl acetate/petroleum ether) to afford 370 mg (62% yield) of the title compound as a white solid. LC-MS (ESI, m/z): [M+H]⁺=221.

### Step 2: 6-Bromo-5-chloro-N,N-bis(4-methoxybenzyl)-4-methylpyridin-2-amine

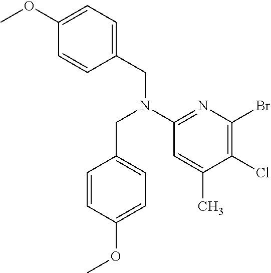 [SMILES: COc1ccc(CN(Cc2ccc(OC)cc2)c2cc(C)c(Cl)c(Br)n2)cc1]

Under nitrogen, to a solution of 6-bromo-5-chloro-4-methyl-pyridin-2-amine (370 mg, 1.67 mmol) in DMF (5 mL) was added 60% NaH (202 mg, 5.05 mmol) at 0° C. The reaction was stirred at room temperature. After 30 min, 1-(chloromethyl)-4-methoxybenzene (656 mg, 4.21 mmol) was added, and the reaction was maintained at room temperature for 1 h. The reaction was quenched with water, and the resulting solution was extracted with EtOAc (3×). The combined organic layers were dried over anhydrous sodium sulfate and concentrated under vacuum. The residue was purified by flash chromatography on silica gel (gradient: 0%-30% ethyl acetate/petroleum ether) to afford 550 mg (71% yield) of the title compound as a white solid. LC-MS (ESI, m/z): [M+H]⁺=461.

### Intermediate 23 and 24: (((2R,7aS)-2-Fluorotetrahydro-1H-pyrrolizin-7a(5H)-yl)methanol and ((2S,7aS)-2-Fluorotetrahydro-1H-pyrrolizin-7a(5H)-yl)methanol

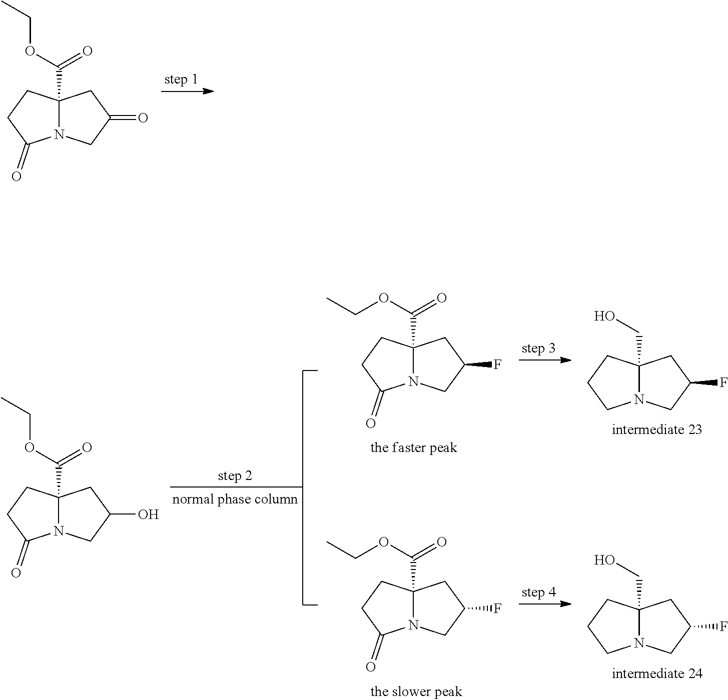

### Step 1: Ethyl (7aS)-2-hydroxy-5-oxotetrahydro-1H-pyrrolizine-7a(5H)-carboxylate

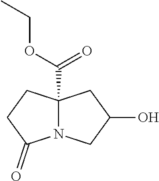

Under nitrogen, to a solution of ethyl (S)-2,5-dioxotetrahydro-1H-pyrrolizine-7a(5H)-carboxylate (1.22 g, 5.68 mmol) in tetrahydrofuran (100 mL) was added NaBH₄ (70.3 mg, 1.85 mmol) at 0° C. After 30 min, the reaction mixture was concentrated under vacuum. The residue was purified by flash chromatography on silica gel (gradient: 0%-6% MeCOH/DCM) to afford the title compound (0.631 g, 62% yield) as a light yellow oil. LC-MS: (ESI, m/z): [M+H]⁺=214.

### Step 2: Ethyl (2R,7aS)-2-fluoro-5-oxotetrahydro-1H-pyrrolizine-7a(5H)-carboxylate and Ethyl (2S,7aS)-2-fluoro-5-oxotetrahydro-1H-pyrrolizine-7a(5H)-carboxylate

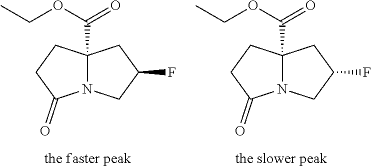

Under nitrogen, to a solution of ethyl (7aS)-2-hydroxy-5-oxotetrahydro-1H-pyrrolizine-7a(5H)-carboxylate (809 mg, 3.80 mmol) was added DAST (923 mg, 5.74 mmol, dissolved in 20 mL DCM) at −15° C. The mixture was warmed to room temperature for 3 hours. The mixture was quenched with EtOH and concentrated under reduced pressure. The residue was purified by flash chromatography on silica gel (gradient: 0%-100% EtOAc/petroleum) to afford 425 mg (52% yield) of ethyl (2R,7aS)-2-fluoro-5-oxotetrahydro-1H-pyrrolizine-7a(5H)-carboxylate as a colorless oil and 219 mg (26.9% yield) of ethyl (2S,7aS)-2-fluoro-5-oxotetrahydro-1H-pyrrolizine-7a(5H)-carboxylate as white solid. LC-MS: (ESI, m/z): [M+H]⁺=216.

Ethyl (2R,7aS)-2-fluoro-5-oxotetrahydro-1H-pyrrolizine-7a(5H)-carboxylate (the faster peak). ¹H NMR (400 MHz, Chloroform-d) δ 5.32 (d, J=19.2 Hz, 1H), 4.26-4.11 (m, 3H), 3.25-3.12 (m, 1H), 2.85-2.59 (m, 3H), 2.48-2.39 (m, 1H), 2.37-2.07 (m, 2H), 1.30 (t, J=7.1 Hz, 3H).

Ethyl (2S,7aS)-2-fluoro-5-oxotetrahydro-1H-pyrrolizine-7a(5H)-carboxylate (the slower peak). ¹H NMR (400 MHz, Chloroform-d) δ 5.47-5.32 (m, 1H), 4.29-4.24 (m, 2H), 4.08-3.96 (m, 1H), 3.46-3.35 (m, 1H), 2.98-2.78 (m, 2H), 2.53-2.42 (m, 2H), 2.20-2.12 (m, 1H), 1.91-1.76 (m, 1H), 1.31 (t, J=7.1 Hz, 3H).

### Step 3: ((2R,7aS)-2-Fluorotetrahydro-1H-pyrrolizin-7a(5H)-yl)methanol

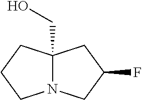 [SMILES: OC[C@@]12CCCN1C[C@H](F)C2]

Under nitrogen, to a solution of ethyl (2R,7aS)-2-fluoro-5-oxotetrahydro-1H-pyrrolizine-7a(5H)-carboxylate (310 mg, 1.44 mmol) in tetrahydrofuran (7 mL) was added LiAlH₄ (3.1 mL, 1M in THF) at 0° C. The mixture was heated to 70° C. for 30 min. After cooling to room temperature, the mixture was quenched with Na₂SO₄·10H₂O with vigorous stirring. The solid was filtered, and the filtrate was evaporated by gently blowing N₂ to afford the title compound (124 mg, crude). LC-MS: (ESI, m/z): [M+H]⁺=160. The crude was used without further purification.

### Step 4: ((2S,7aS)-2-Fluorotetrahydro-1H-pyrrolizin-7a(5H)-yl)methanol

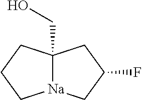

Analogous to method described in step 3, the title compound was prepared from ethyl (2S,7aS)-2-fluoro-5-oxotetrahydro-1H-pyrrolizine-7a(5H)-carboxylate. LC-MS: (ESI, m/z): [M+H]⁺=160. The crude was used without further purification.

### Intermediate 25: 6-Bromo-N,N-bis(4-methoxybenzyl)-5-(trifluoromethyl)pyridin-2-amine

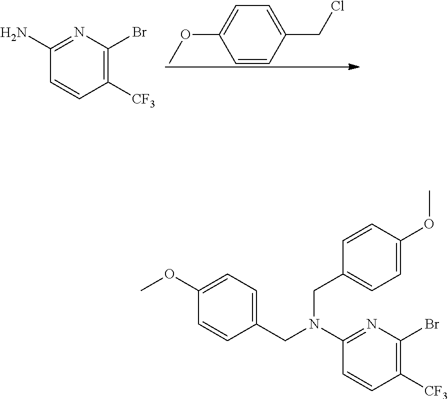

Under nitrogen, to a solution of 6-bromo-5-(trifluoromethyl) pyridin-2-amine (500 mg, 2.08 mmol) in N,N-dimethylformamide (5 mL) was added 60% NaH (416 mg, 10.4 mmol) at 0° C. The resulting solution was warmed to room temperature. After 30 min, 1-(chloromethyl)-4-methoxybenzene (812 mg, 5.20 mmol) was added dropwise, and the resulting solution was stirred for 3 h at room temperature. The reaction was quenched with saturated aqueous NH₄Cl solution, and the resulting mixture was extracted with EtOAc (3×). The combined organic layers were washed with brine, dried over anhydrous sodium sulfate, filtered, and concentrated. The residue was purified by flash chromatography on silica gel (gradient: 0%-20% EtOAc/petroleum ether) to yield 645 mg (64% yield) of the title compound as a white solid. LC-MS: (ESI, m/z): [M+H]⁺=481/483.

### Intermediate 26 [2]: 6-Bromo-N,N-bis(4-methoxybenzyl)-5-(trifluoromethyl)pyridin-2-amine

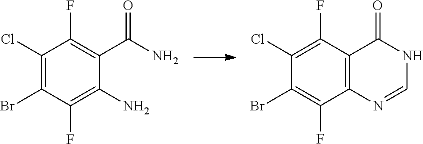 [SMILES: NC(=O)c1c(N)c(F)c(Br)c(Cl)c1F ; O=c1[nH]cnc2c(F)c(Br)c(Cl)c(F)c12]

To a solution of 2-amino-4-bromo-5-chloro-3,6-difluorobenzamide (5.00 g, 17.5 mmol, intermediate 2, step 6) in triethyl orthoformate (100 mL) was added AcOH (10 mL). The resulting mixture was stirred at 80° C. for 1 h. The solvent was removed under vacuum. The residue was diluted with EtOAc/DCM (1/5, 150 mL). The solids were collected by filtration and dried under vacuum to afford the title compound (4 g, crude) as a white solid which was used for next reaction without further purification. LC-MS: (ESI, m/z): [M+H]⁺=295. ¹H NMR (400 MHz, DMSO-d₆) δ 12.76 (s, 1H), 8.22 (s, 1H).

### Intermediate 27: 6-Bromo-N,N-bis(4-methoxybenzyl)-5-(trifluoromethyl)pyridin-2-amine

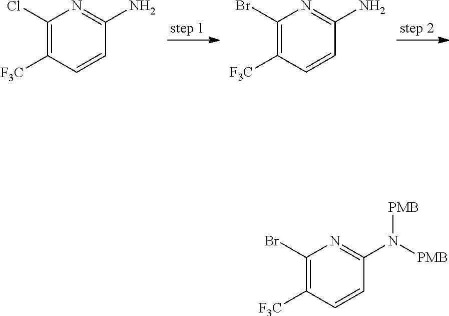

### Step 1: 6-Bromo-5-(trifluoromethyl)pyridin-2-amine

Under nitrogen, a solution of 6-chloro-5-(trifluoromethyl)pyridin-2-amine (1000 mg, 5.09 mmol) and HBr in acetic acid (10 mL, 33% w/w) was stirred for 48 h at 130° C. (steel tank). The reaction was cooled to room temperature and concentrated under vacuum. The residue was diluted with H₂O (30 mL), adjusted pH=8˜9 with Na₂CO₃ (aq.) and extracted with EtOAc. The combined organic layers were dried over anhydrous sodium sulfate and concentrated under vacuum. The residue was purified by flash chromatography on silica gel (gradient: 0%-10% EtOAc/petroleum ether) to afford the title compound (1106 mg, 86.8% yield). LC-MS: (ESI, m/z): [M+H]⁺=241/243.

### Step 2: 6-Bromo-N,N-bis(4-methoxybenzyl)-5-(trifluoromethyl)pyridin-2-amine

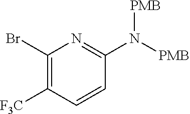

Under nitrogen, to a solution of 6-bromo-5-(trifluoromethyl) pyridin-2-amine (500 mg, 2.08 mmol) in DMF (5 mL) was added NaH (416 mg, 10.4 mmol, 60% suspend in oil) at 0° C. The resulting solution was warmed to room temperature and stirred for 0.5 h. Then 1-(chloromethyl)-4-methoxybenzene (812 mg, 5.20 mmol) was added dropwise at room temperature. The resulting solution was stirred for 3 h at room temperature. The reaction was quenched with saturated NH₄Cl solution, extracted with EtOAc (3×). The combined organic layers were washed with brine, dried over anhydrous sodium sulfate and concentrated under vacuum. The residue was purified by flash chromatography on silica gel (gradient: 0%-20% EtOAc/petroleum ether) to yield 645 mg (64% yield) of the title compound as a white solid. LC-MS: (ESI, m/z): [M+H]⁺=481/483.

### Intermediate 28 [5]: 7-Bromo-6-fluoro-1-methyl-1H-indazole

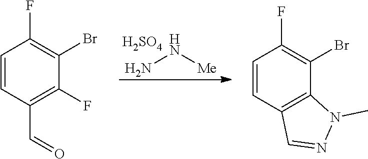

Under nitrogen, a solution of 3-bromo-2,4-difluoro-benzaldehyde (500 mg, 2.26 mmol), 1-methylhydrazine sulfuric acid salt (1.63 g, 11.3 mmol) and K₂CO₃ (3.12 g, 22.6 mmol) in NMP (15 mL) was stirred at 200° C. for 2 h under microwave irradiation. The reaction system was cooled to room temperature, diluted with water (100 mL), extracted with EtOAc. The combined organic layers were dried over anhydrous sodium sulfate and concentrated under vacuum. The residue was purified by flash chromatography on silica gel eluting with EtOAc/petroleum ether (0-10%) to afford the title compound (333.2 mg, 64.3% yield) as a white solid. LC-MS: (ESI, m/z): [M+H]⁺=229/231; ¹H NMR (400 MHz, DMSO-d₆) δ 8.13 (s, 1H), 7.80 (dd, J=8.7, 5.0 Hz, 1H), 7.15 (t, J=8.9 Hz, 1H), 4.31 (s, 3H).

### Intermediate 29: tert-Butyl (1R,2S,5S)-2-((S)-1-hydroxyethyl)-3,8-diazabicyclo[3.2.1]octane-8-carboxylate

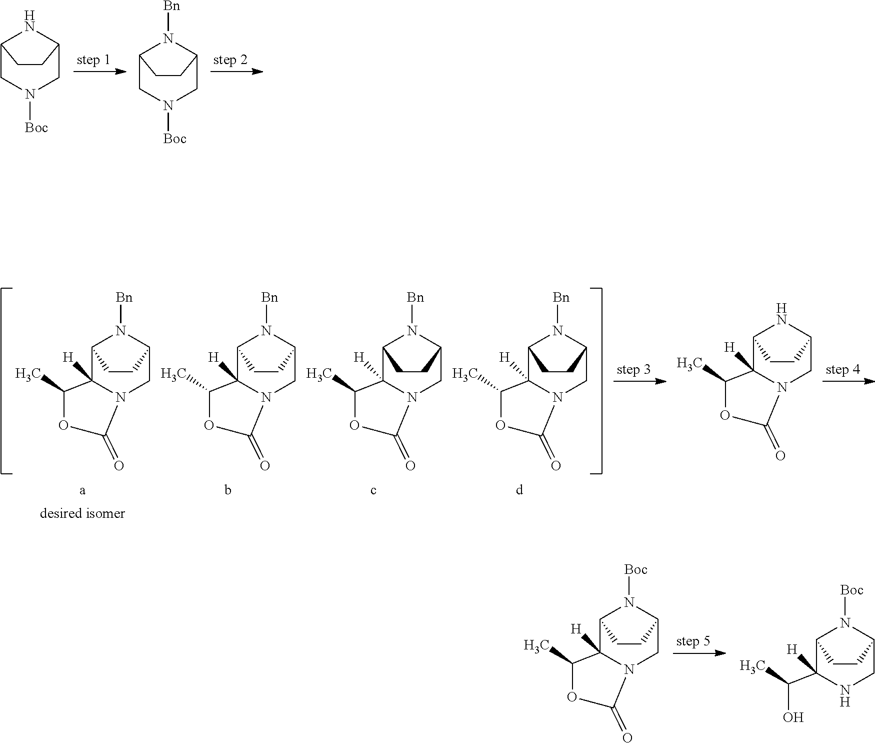

### Step 1: tert-Butyl 8-benzyl-3,8-diazabicyclo[3.2.1]octane-3-carboxylate

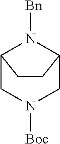 [SMILES: CC(C)(C)OC(=O)N1CC2CCC(C1)N2Cc1ccccc1]

Under nitrogen, to a solution of tert-butyl 3,8-diazabicyclo[3.2.1]octane-3-carboxylate (5.00 g, 23.5 mmol) in N,N-dimethylformamide (50 mL) were added K₂CO₃ (6.51 g, 47.1 mmol) and (bromomethyl)benzene (6.01 g, 35.1 mmol) at 0° C. Stirred for 1 h at room temperature. The reaction mixture was poured into ice water, and extracted with EtOAc. The combined organic layers were dried over anhydrous Na₂SO₄ and concentrated under vacuum. The residue was purified by flash chromatography on silica gel (gradient: 0-30% EtOAc/petroleum ether) to yield 7 g (98.3% yield) the title compound as a white solid. LC-MS: (ESI, m/z): [M+H]⁺=303.

### Step 2: (1S,6S,9R,9aS)-10-Benzyl-1-methylhexahydro-1H,3H-6,9-epiminooxazolo[3,4-a]azepin-3-one

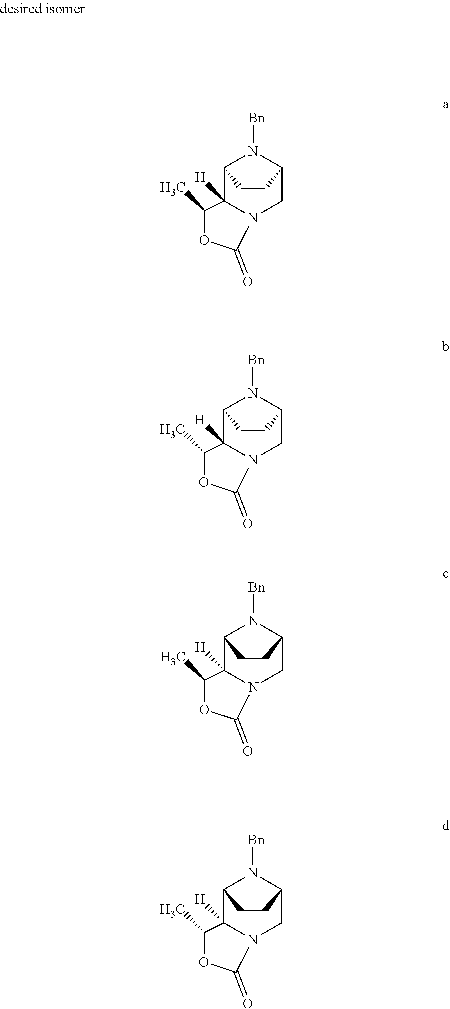 [SMILES: C[C@@H]1OC(=O)N2C[C@H]3CC[C@@H]([C@@H]12)N3Cc1ccccc1 ; C[C@H]1OC(=O)N2C[C@H]3CC[C@@H]([C@@H]12)N3Cc1ccccc1 ; C[C@@H]1OC(=O)N2C[C@@H]3CC[C@H]([C@H]12)N3Cc1ccccc1 ; C[C@H]1OC(=O)N2C[C@@H]3CC[C@H]([C@H]12)N3Cc1ccccc1]

Under nitrogen, to a solution of tert-butyl 8-benzyl-3,8-diazabicyclo[3.2.1]octane-3-carboxylate (7.0 g, 23.1 mmol) and TMEDA (5.38 g, 46.3 mmol) in diethyl ether (70 mL) was added s-BuLi (35.6 mL, 46.3 mmol, 1.3 M in hexane) dropwise at −78° C. The resulting solution was stirred for 1.5 h at −78° C. Then acetaldehyde (2.55 g, 57.8 mmol) was added at −78° C. The reaction was allowed to warm to room temperature gradually and stirred overnight. The mixture was quenched with NH₄Cl (aq.) and extracted with EtOAc. The combined organic layers were dried over anhydrous Na₂SO₄ and concentrated under vacuum. The residue was purified by flash chromatography on silica gel (0-50% EtOAc in petroleum ether) to yield 5.1 g mixture of 4 diastereoisomers. The mixture was separated by Prep-SFC (Column: CHIRALPAK IH, 3*25 cm, 5 μm; Mobile Phase A: CO₂, Mobile Phase B: IPA (0.5% 2M NH₃-MeCOH); Flow rate: 70 mL/min; Gradient: isocratic 35% B; Column Temperature (° C.): 35; Back Pressure (bar): 100; Wave Length: 220 nm; RT1 (min): 6.31; RT2 (min): 8.33; Sample Solvent: MeOH—Preparative; Injection Volume: 1.9 mL; Number Of Runs: 50) to yield the compound a (1.39 g, 22% yield) (the first peak) and compound d (1.47 g, 23.3% yield) (the third peak) and mixture of compound b and c (the second peak). The mixture of compound b and c was re-separated by Prep-SFC (Column: CHIRALPAK IH, 5*25 cm, 5 μm; Mobile Phase A: CO₂, Mobile Phase B: IPA (0.5% 2M NH₃-MeOH); Flow rate: 200 mL/min; Gradient: isocratic 50% B; Column Temperature (° C.): 35; Back Pressure (bar): 100; Wave Length: 220 nm; RT1 (min): 5.73; RT2 (min): 8.44; Sample Solvent: MeOH—Preparative; Injection Volume: 10 mL; Number Of Runs: 6) to yield compound b (0.500 g, 7.9% yield) (the faster peak) and compound c (0.430 g, 6.8% yield) (the slower peak) as yellow solid. LC-MS: (ESI, m/z): [M+H]⁺=273. Compound a is desired isomer.

### Step 3: (1S,6S,9R,9aS)-1-Methylhexahydro-1H,3H-6,9-epiminooxazolo[3,4-a]azepin-3-one

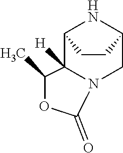

A solution of (1S,6S,9R,9aS)-10-Benzyl-1-methylhexahydro-1H,3H-6,9-epiminooxazolo[3,4-a]azepin-3-one (1.00 g, 3.67 mmol) (the compound a of previous step) and Pd/C (500 mg, 10%) in methyl alcohol (15 mL) was was stirred for 1 h at room temperature under an atmosphere of hydrogen gas. The catalyst was filtered off. The filtrate was concentrated under vacuum to yield 658 mg (crude) the title compound as a yellow oil which was used for next step without further purification. LC-MS: (ESI, m/z): [M+H]⁺=183.

### Step 4: tert-Butyl (1S,6S,9R,9aS)-1-methyl-3-oxohexahydro-1H,3H-6,9-epiminooxazolo[3,4-a]azepine-10-carboxylate

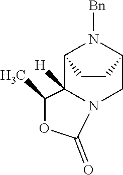 [SMILES: C[C@@H]1OC(=O)N2C[C@H]3CC[C@@H]([C@@H]12)N3Cc1ccccc1]

A solution of (1 S,6S,9R,9aS)-1-methylhexahydro-1H,3H-6,9-epiminooxazolo[3,4-a]azepin-3-one (658 mg, 3.61 mmol), (Boc)₂O (1.18 g, 5.41 mmol) and DIPEA (1.4 g, 10.8 mmol) in dichloromethane (10 mL) was stirred for 30 min at room temperature. The reaction system was quenched with water, extracted with DCM. The combined organic layers were dried over anhydrous Na₂SO₄ and concentrated under vacuum. The residue was purified by flash chromatography on silica gel (gradient: 0-100% EtOAc/petroleum ether) to yield the title compound (920 mg, 90.2% yield) as a white solid. LC-MS: (ESI, m/z): [M+H]⁺=283.

### Step 5: tert-Butyl (1R,2S,5S)-2-((S)-1-hydroxyethyl)-3,8-diazabicyclo[3.2.1]octane-8-carboxylate

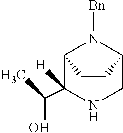 [SMILES: C[C@H](O)[C@H]1NC[C@H]2CC[C@@H]1N2Cc1ccccc1]

A solution of tert-butyl (1S,6S,9R,9aS)-1-methyl-3-oxohexahydro-1H,3H-6,9-epiminooxazolo[3,4-a]azepine-10-carboxylate (900 mg, 3.19 mmol) and NaOH (1.28 g, 32.0 mmol) in ethanol (12 mL) and water (4 mL) was stirred for 1 h at 80° C. The reaction solution was cooled to room temperature and diluted with water, extracted with DCM. The combined organic layers were dried over anhydrous Na₂SO₄ and concentrated under vacuum to yield 815 mg (crude) as an oil which was used for next step without further purification. LC-MS: (ESI, m/z): [M+H]⁺=257. ¹H NMR (300 MHz, DMSO-d₆) δ 4.54 (s, 1H), 3.94 (d, J=5.1 Hz, 1H), 3.82 (s, 1H), 2.73 (d, J=11.3 Hz, 1H), 2.60 (d, J=11.5 Hz, 1H), 2.41 (d, J=8.1 Hz, 1H), 2.15 (s, 1H), 179-1.67 (m, 3H), 1.56 (s, 1H), 1.40 (s, 9H), 1.04 (d, J=6.3 Hz, 3H).

### Intermediate 30: tert-Butyl (2R,5S,5aS,6S,9R)-2-(6-(bis(4-methoxybenzyl)amino)-4-methyl-3-(trifluoromethyl)pyridin-2-yl)-3,13-dichloro-1-fluoro-5-methyl-5a,6,7,8,9,10-hexahydro-5H-6,9-epiminoazepino[2′,1′:3,4][1,4]oxazepino[5,6,7-de]quinazoline-15-carboxylate

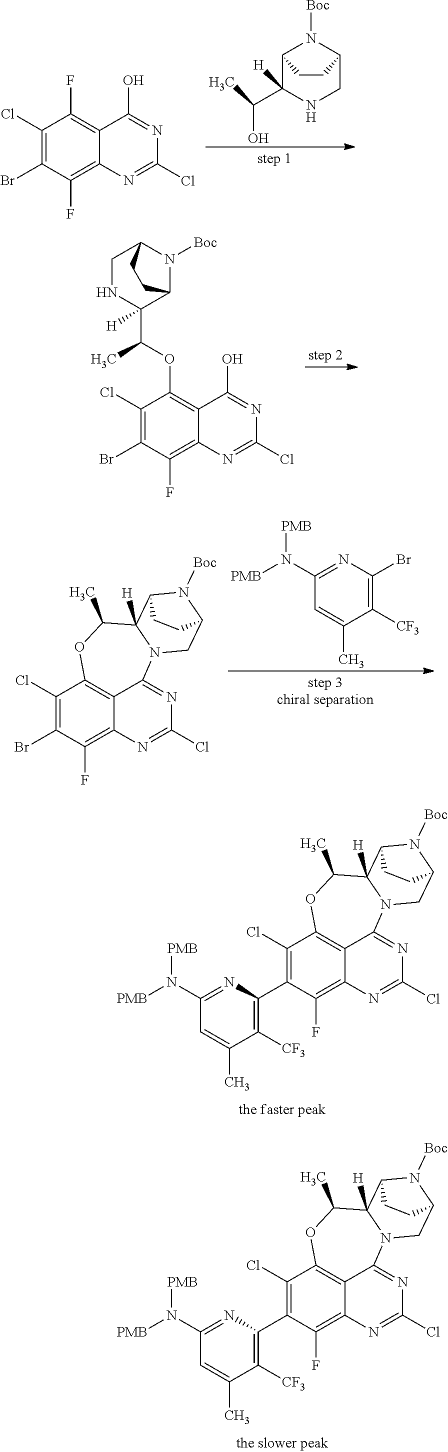

### Step 1: tert-Butyl (1 S,2S,5R)-2-((S)-1-((7-bromo-2,6-dichloro-8-fluoro-4-hydroxyquinazolin-5-yl)oxy)ethyl)-3,8-diazabicyclo[3.2.1]octane-8-carboxylate

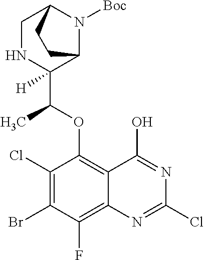 [SMILES: C[C@H](Oc1c(Cl)c(Br)c(F)c2nc(Cl)nc(O)c12)[C@H]1NC[C@H]2CC[C@@H]1N2C(=O)OC(C)(C)C]

Under nitrogen, to a solution of tert-butyl (1R,2S,5S)-2-((S)-1-hydroxyethyl)-3,8-diazabicyclo[3.2.1]octane-8-carboxylate (6.00 g, 23.4 mmol, intermediate 29) in tetrahydrofuran (200 mL) was added NaH (5.00 g, 125 mmol, 60% suspend in oil) at 0° C. The resulting solution was stirred for 30 min at 0° C. Then 7-bromo-2,6-dichloro-5,8-difluoro-quinazolin-4-ol (15.4 g, 46.6 mmol, intermediate 2) was added. The reaction system was stirred for 1 h at room temperature. The mixture was quenched with saturated NH₄Cl, extracted with EtOAc. The combined organic layers were dried over anhydrous Na₂SO₄ and concentrated under vacuum to yield the title compound 17.7 g (crude) as a brown solid, which was used for next step without further purification. LC-MS: (ESI, m/z): [M+H]⁺=565.

### Step 2: tert-Butyl (5S,5aS,6S,9R)-2-bromo-3,13-dichloro-1-fluoro-5-methyl-5a,6,7,8,9,10-hexahydro-5H-6,9-epiminoazepino[2′,1′:3,4][1,4]oxazepino[5,6,7-de]quinazoline-15-carboxylate

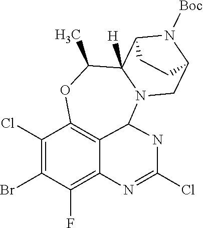 [SMILES: C[C@@H]1Oc2c(Cl)c(Br)c(F)c3c2C(NC(Cl)=N3)N2C[C@H]3CC[C@@H]([C@@H]12)N3C(=O)OC(C)(C)C]

A solution of tert-butyl (1 S,2S,5R)-2-((S)-1-((7-bromo-2,6-dichloro-8-fluoro-4-hydroxyquinazolin-5-yl)oxy)ethyl)-3,8-diazabicyclo[3.2.1]octane-8-carboxylate (17.7 g, 31.2 mmol), BOPCl (31.8 g, 125.5 mmol) and DIPEA (60.7 g, 470 mmol) in dichloromethane (200 mL) was stirred overnight at room temperature. The solid was filtered off. The filtrate was concentrated under vacuum. The residue was purified by flash chromatography on silica gel (gradient: 0-30% EtOAc/petroleum ether) to yield 7.2 g (42% yield) of the title compound as a yellow solid. LC-MS: (ESI, m/z): [M+H]⁺=547.

### Step 3: tert-Butyl (2R,5S,5aS,6S,9R)-2-(6-(bis(4-methoxybenzyl)amino)-4-methyl-3-(trifluoromethyl)pyridin-2-yl)-3,13-dichloro-1-fluoro-5-methyl-5a,6,7,8,9,10-hexahydro-5H-6,9-epiminoazepino[2′,1′:3,4][1,4]oxazepino[5,6,7-de]quinazoline-15-carboxylate

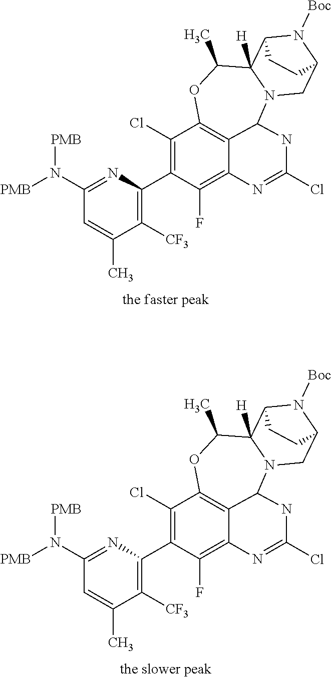

Under nitrogen, to a solution of tert-butyl (5S,5aS,6S,9R)-2-bromo-3,13-dichloro-1-fluoro-5-methyl-5a,6,7,8,9,10-hexahydro-5H-6,9-epiminoazepino[2′,1′:3,4][1,4]oxazepino[5,6,7-de]quinazoline-15-carboxylate (510 mg, 0.930 mmol) in tetrahydrofuran (3.2 mL) was added iPrMgCl·LiCl (0.93 mL, 1.3 M in THF) at −78° C. The resulting solution was stirred for 10 min at −78° C. Then ZnCl₂ (2 M in 2-MeTHF) (0.93 mL, 1.86 mmol) was added at −78° C. and stirred for 10 min. The mixture was warmed to room temperature and stirred for 20 min at this temperature. The mixture was added to a solution of 6-bromo-N,N-bis[(4-methoxyphenyl)methyl]-4-methyl-5-(trifluoromethyl)pyridin-2-amine (461 mg, 0.930 mmol, intermediate 4) and Pd(PPh₃)₂Cl₂ (32.8 mg, 0.0500 mmol) in tetrahydrofuran (5 mL). The resulting solution was stirred overnight at 50° C. Concentrated under vacuum. The residue was purified by flash chromatography on silica gel (gradient: 0-40% EtOAc/petroleum ether) to yield 350 mg title compound (mixture of 2 atropisomers) as a yellow solid. The mixture was separated by PREP_CHIRAL_HPLC (Column: CHIRAL ART Cellulose-SB, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 10% B to 10% B in 22 min; Wave Length: 220/254 nm; RT1 (min): 14.03; RT2 (min): 16.79; Sample Solvent: EtOH—HPLC; Injection Volume: 0.3 mL; Number Of Runs: 15) to yield 80 mg of the faster peak and 110 mg of the slower peak as yellow solids. LC-MS: (ESI, m/z): [M+H]⁺=883.

The faster peak: ¹H NMR (300 MHz, DMSO-d₆) δ 7.19-7.08 (m, 4H), 6.90-6.78 (m, 5H), 5.11 (d, J=13.1 Hz, 1H), 4.77 (d, J=15.9 Hz, 2H), 4.59 (s, 1H), 4.48 (d, J=15.8 Hz, 2H), 4.31 (s, 1H), 4.14 (s, 1H), 4.02 (d, J=9.5 Hz, 1H), 3.72 (s, 6H), 3.09 (d, J=13.1 Hz, 1H), 2.40 (d, J=2.1 Hz, 3H), 1.99-1.68 (m, 4H), 1.54 (d, J=6.2 Hz, 3H), 1.46 (s, 9H).

The slower peak: ¹H NMR (300 MHz, DMSO-d₆) δ 7.21-7.09 (m, 4H), 6.92-6.80 (m, 5H), 5.13 (d, J=13.2 Hz, 1H), 4.79-4.51 (m, 5H), 4.31 (d, J=5.2 Hz, 1H), 4.12 (s, 1H), 4.05 (d, J=9.6 Hz, 1H), 3.73 (s, 6H), 3.14 (d, J=13.2 Hz, 1H), 2.38 (d, J=2.2 Hz, 3H), 1.99-1.68 (m, 4H), 1.53 (d, J=6.2 Hz, 3H), 1.47 (s, 9H).

### Intermediate 31: tert-Butyl (2R,5S,5aS,6S,9R)-2-(6-(bis(4-methoxybenzyl)amino)-3-(trifluoromethyl)pyridin-2-yl)-3,13-dichloro-1-fluoro-5-methyl-5a,6,7,8,9,10-hexahydro-5H-6,9-epiminoazepino[2′,1′:3,4][1,4]oxazepino[5,6,7-de]quinazoline-15-carboxylate

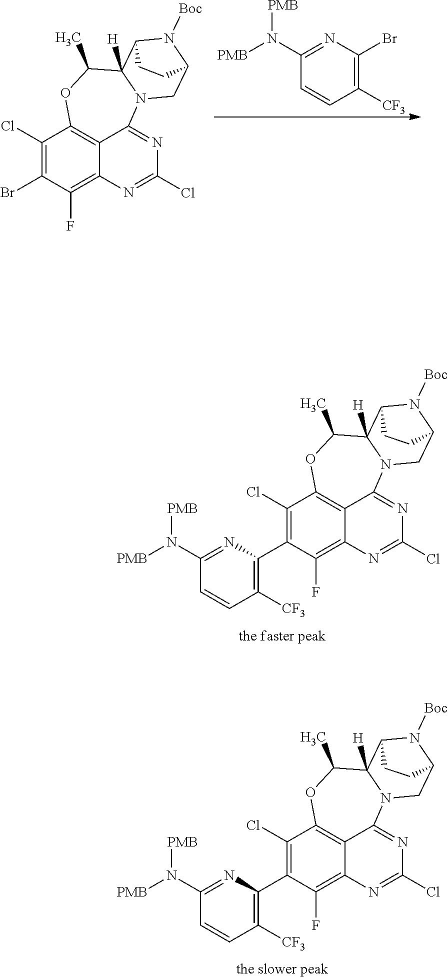

Under nitrogen, to a solution of tert-butyl (5S,5aS,6S,9R)-2-bromo-3,13-dichloro-1-fluoro-5-methyl-5a,6,7,8,9,10-hexahydro-5H-6,9-epiminoazepino[2′,1′:3,4][1,4]oxazepino[5,6,7-de]quinazoline-15-carboxylate (1.70 g, 3.10 mmol, Intermediate 30, step 2) in tetrahydrofuran (10 mL) was added iPrMgCl·LiCl (3.1 mL, 4.03 mmol, 1.3 M in THF) at −78° C. The resulting solution was stirred for 10 min at −78° C. Then ZnCl₂ (3.1 mL, 6.20 mmol, 2 M in 2-MeTHF) was added at −78° C. The solution was stirred for 10 min at −78° C. and warmed to room temperature and stirred addition 20 min at room temperature. The mixture was added to a solution of 6-bromo-N,N-bis[(4-methoxyphenyl)methyl]-5-(trifluoromethyl)pyridin-2-amine (1.50 g, 3.12 mmol, intermediate 27) and Pd(PPh₃)₂Cl₂ (109 mg, 0.160 mmol) in tetrahydrofuran (17 mL) at room temperature. The resulting solution was stirred overnight at 50° C., and then concentrated under vacuum. The crude was purified by flash chromatography on silica gel (gradient: 0-30% EtOAc/petroleum ether) to yield 1.2 g of the title compound (mixture of 2 atropisomers). The mixture was separated by PREP_SFC (Column: Lux 5 μm Cellulose-4, 3*25 cm, 5 μm; Mobile Phase A: CO2, Mobile Phase B: MeCOH:ACN=1:1 (0.1% 2M NH₃-MeOH); Flow rate: 70 mL/min; Gradient: isocratic 40% B; Column Temperature (° C.): 35; Back Pressure (bar): 100; Wave Length: 220 nm; RT1 (min): 4.34; RT2 (min): 5.16; Sample Solvent: MeOH—Preparative; Injection Volume: 1 mL; Number Of Runs: 75) to yield 537 mg of the faster peak and 460 mg of the slower peak as yellow solids. LC-MS: (ESI, m/z): [M+H]⁺=869.

### Intermediate 32: ((2S,4R)-4-Fluoro-1-methylpyrrolidin-2-yl)methanol

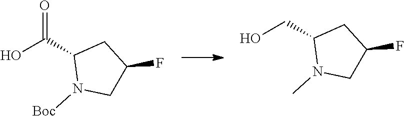

Under nitrogen, to a solution (2S,4R)-1-(tert-butoxycarbonyl)-4-fluoropyrrolidine-2-carboxylic acid (60.0 g, 257 mmol) in tetrahydrofuran (1000 mL) was added LiAlH₄ (19.6 g, 515 mmol) at 0° C. The reaction mixture was stirred at room temperature for 1 h, and then stirred at 60° C. for 2 h. The reaction mixture was cooled over ice bath and water (20 mL) was slowly added to quench the reaction, followed 20% aqueous NaOH solution (20 mL) and 20 mL water. The solids were filtered off and filtrate was concentrated under vacuum. The residue was redissolved in DCM, dried over anhydrous Na₂SO₄ and concentrated under vacuum. The residue was purified by flash chromatography on silica gel (gradient: 0%-15% MeCOH in DCM (0.1% TEA)) to yield 12.4 g (36% yield) of the title compound as a yellow oil. LC-MS: (ESI, m/z): [M+H]⁺=134. 1H NMR (300 MHz, DMSO-d₆, ppm) δ 5.25-4.97 (m, 1H), 4.44 (dd, J=6.0, 5.0 Hz, 1H), 3.50-3.22 (m, 3H), 2.57-2.50 (m, 1H), 2.46-2.31 (m, 1H), 2.30 (s, 3H), 2.07-1.87 (m, 1H), 1.87-1.60 (m, 1H).

### Intermediate 33: ((3S)-3-Fluoro-1-azabicyclo[3.2.0]heptan-5-yl)methanol

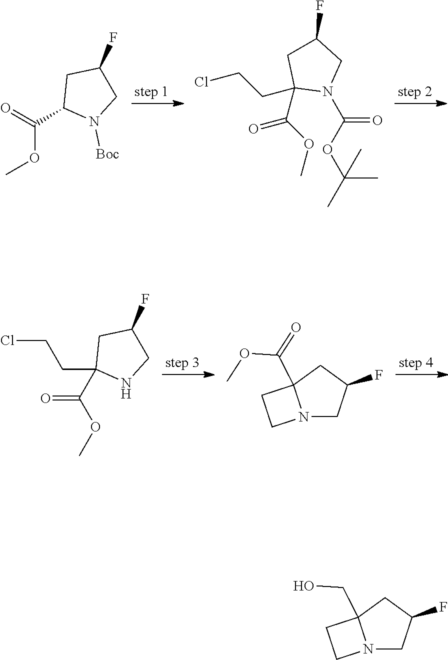 [SMILES: COC(=O)[C@@H]1C[C@@H](F)CN1C(=O)OC(C)(C)C ; COC(=O)C1(CCCl)C[C@@H](F)CN1C(=O)OC(C)(C)C ; COC(=O)C1(CCCl)C[C@@H](F)CN1 ; COC(=O)C12CCN1C[C@H](F)C2 ; OCC12CCN1C[C@H](F)C2]

### Step 1: 1-(tert-Butyl) 2-methyl (4R)-2-(2-chloroethyl)-4-fluoropyrrolidine-1,2-dicarboxylate

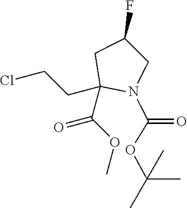 [SMILES: COC(=O)C1(CCCl)C[C@@H](F)CN1C(=O)OC(C)(C)C]

Under nitrogen, to a solution of 1-(tert-butyl) 2-methyl (2S,4R)-4-fluoropyrrolidine-1,2-dicarboxylate (5.00 g, 20.2 mmol) and HMPA (10.9 g, 60.9 mmol) in tetrahydrofuran (70 mL) was added LiHMDS in THF (60.7 mL, 60.7 mmol, 1 M) at −78° C. The resulting solution was stirred at −78° C. for 1 h. Then 1-bromo-2-chloroethane (8.36 mL, 100 mmol) was added. The resulting solution was stirred at room temperature for 1 h. The mixture was quenched with NH₄Cl (aq.), extracted with EtOAc. The combined organic layers were dried over Na₂SO₄ and concentrated under vacuum. The residual was purified by flash chromatography on silica gel (gradient: 0%-100% EtOAc/petroleum ether) to yield 1.26 g (20.1% yield) of the title compound as a colorless oil. LC-MS: (ESI, m/z): [M+H]⁺=310.

### Step 2: Methyl (4R)-2-(2-chloroethyl)-4-fluoropyrrolidine-2-carboxylate

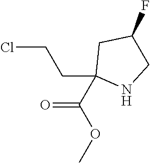

To a solution of 1-(tert-Butyl) 2-methyl (4R)-2-(2-chloroethyl)-4-fluoropyrrolidine-1,2-dicarboxylate (1.26 g, 3.87 mmol) in dichloromethane (10 mL) was added TFA (5 mL). The resulting solution was stirred at room temperature for 30 min. Solvent was evaporated under vacuum to yield 2 g (crude) of the title compound as a yellow oil which was used for next step without further purification.. LC-MS: (ESI, m/z): [M+H]⁺=210.

### Step 3: Methyl (3S)-3-fluoro-1-azabicyclo[3.2.0]heptane-5-carboxylate

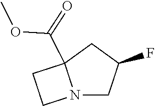

A solution of methyl (4R)-2-(2-chloroethyl)-4-fluoropyrrolidine-2-carboxylate (2.00 g, 3.82 mmol) and K₂CO₃ (1.60 g, 11.6 mmol) in acetonitrile (30 mL) was stirred at 85° C. for 1 h. The solids were filtered off. The filtrate was concentrated under vacuum to yield 2.9 g (crude) of the title compound as a yellow oil which was used for next step without further purification. LC-MS: (ESI, m/z): [M+H]⁺=174.

### Step 4: ((3S)-3-Fluoro-1-azabicyclo[3.2.0]heptan-5-yl)methanol

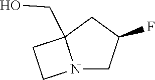

Under nitrogen, to a solution of methyl (3S)-3-fluoro-1-azabicyclo[3.2.0]heptane-5-carboxylate (2.90 g, 16.7 mmol) in tetrahydrofuran (20 mL) was added LiAlH₄ in THF (16.7 mL, 16.7 mmol, 1 M) at 0° C. The resulting solution was stirred at 0° C. for 30 min. The mixture was quenched by Na₂SO₄·10H₂O and filtrated. The solvent was remove by blowing N₂ (volatile) to yield 2 g (crude) as a yellow oil which was used for next step without further purification. LC-MS: (ESI, m/z): [M+H]⁺=146.

### Intermediate 34: ((3S)-2′,2′-Difluoro-1-azaspiro[bicyclo[3.2.0]heptane-3,1′-cyclopropan]-5-yl)methanol (two single unknown isomers) and ((3R)-2′,2′-difluoro-1-azaspiro[bicyclo[3.2.0]heptane-3,1′-cyclopropan]-5-yl)methanol (two single unknown isomers)

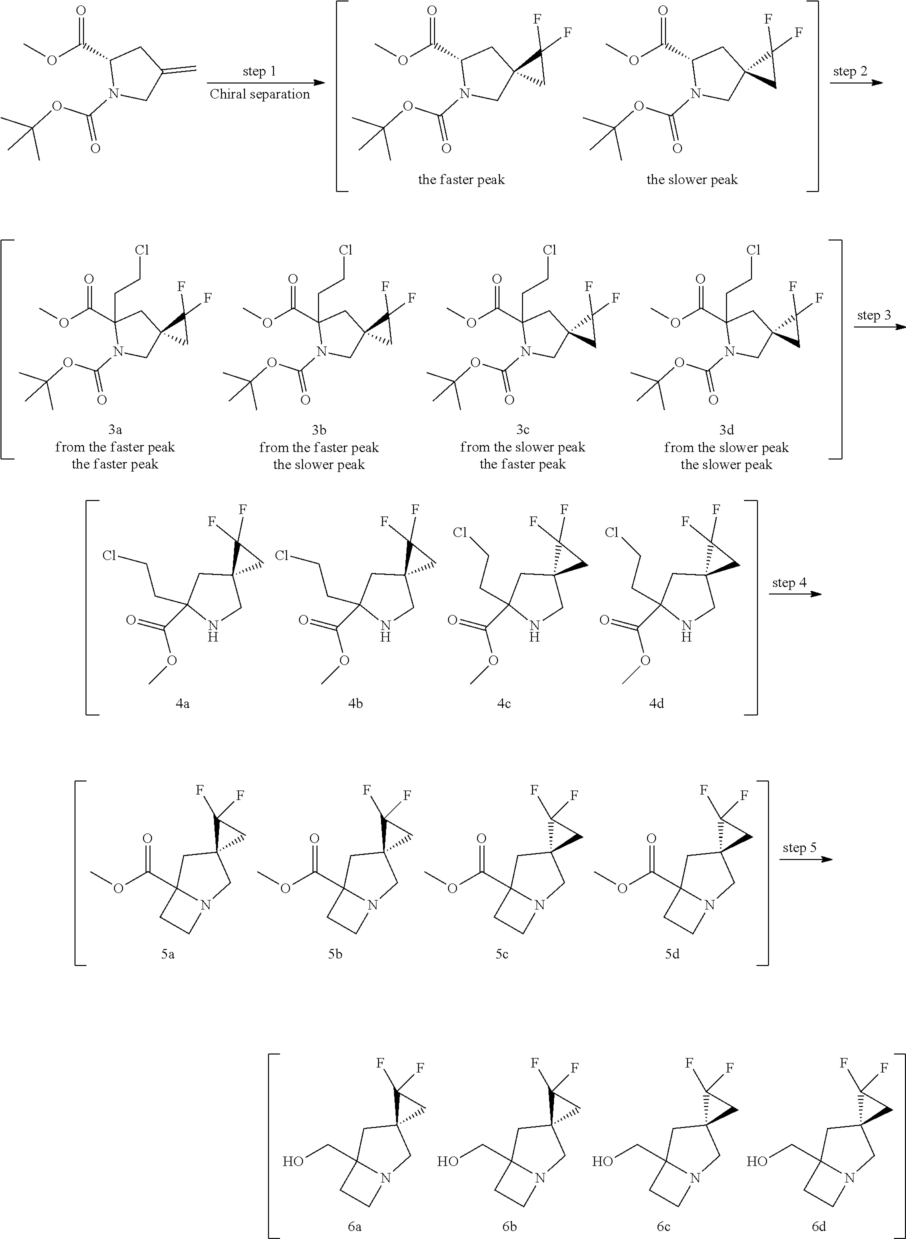

### Step 1: 5-(tert-butyl) 6-Methyl (3S,6S)-1,1-difluoro-5-azaspiro[2.4]heptane-5,6-dicarboxylate and 5-(tert-butyl) 6-methyl (3R,6S)-1,1-difluoro-5-azaspiro[2.4]heptane-5,6-dicarboxylate

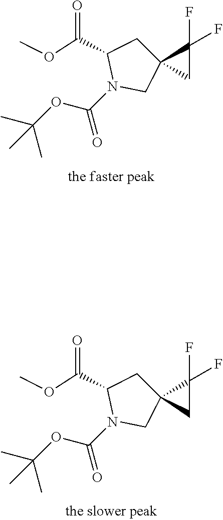 [SMILES: COC(=O)[C@@H]1C[C@@]2(CN1C(=O)OC(C)(C)C)CC2(F)F ; COC(=O)[C@@H]1C[C@]2(CN1C(=O)OC(C)(C)C)CC2(F)F]

Under nitrogen, a solution of 1-(tert-butyl) 2-methyl (S)-4-methylenepyrrolidine-1,2-dicarboxylate (5.00 g, 20.7 mmol) in tetrahydrofuran (70 mL) was added TMSCF₃ (10.3 g, 72.5 mmol) and NaI (1.55 g, 10.3 mmol) at room temperature. The resulting solution was stirred for 2 h at 60° C. The reaction was concentrated under vacuum. The residue was diluted with water, extracted with DCM. The combined organic layers were dried over anhydrous sodium sulfate and then concentrated under vacuum. The residue was purified by flash chromatography on silica gel (gradient: 0-20% EtOAc/petroleum ether) to afford 4.6 g mixture of the title compound as a yellow oil. The mixture was separated by Chiral-Prep-HPLC (Column: CHIRALPAK IH, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 5% B to 5% B in 8 min; Wave Length: 220/254 nm; R_{T1} (min): 6.313; R_{T2} (min): 7.224; Sample Solvent: EtOH—HPLC; Injection Volume: 0.5 mL; Number Of Runs: 32.) to afford 1.14 g (the faster peak) and 600 mg (the slower peak) as yellow oil. LC-MS: (ESI, m/z): [M+H]⁺=292.

The faster peak: ¹H NMR (400 MHz, DMSO-d₆) δ 4.44-4.31 (m, 1H), 3.67 (d, J=9.2 Hz, 3H), 3.59-3.49 (m, 1H), 3.45-3.36 (m, 1H), 2.61-2.47 (m, 1H), 2.02-1.85 (m, 1H), 1.71-1.49 (m, 2H), 1.37 (d, J=22.9 Hz, 9H).

The slower peak: ¹H NMR (400 MHz, DMSO-d₆) δ 4.42-4.31 (m, 1H), 3.64 (d, J=9.2 Hz, 3H), 3.50-3.36 (m, 2H), 2.61-2.51 (m, 1H), 1.93-1.78 (m, 1H), 1.71-1.41 (m, 2H), 1.37 (d, J=25.2 Hz, 9H).

### Step 2: 5-(tert-butyl) 6-Methyl (3S)-6-(2-chloroethyl)-1,1-difluoro-5-azaspiro[2.4]heptane-5,6-dicarboxylate (two single unknown isomers) and 5-(tert-butyl) 6-methyl (3R)-6-(2-chloroethyl)-1,1-difluoro-5-azaspiro[2.4]heptane-5,6-dicarboxylate (two single unknown isomers)

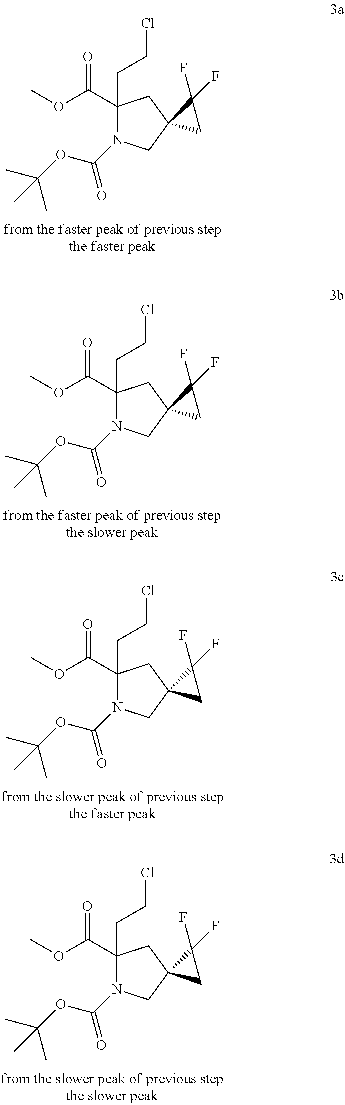 [SMILES: COC(=O)C1(CCCl)C[C@@]2(CN1C(=O)OC(C)(C)C)CC2(F)F ; COC(=O)C1(CCCl)C[C@@]2(CN1C(=O)OC(C)(C)C)CC2(F)F ; COC(=O)C1(CCCl)C[C@]2(CN1C(=O)OC(C)(C)C)CC2(F)F ; COC(=O)C1(CCCl)C[C@]2(CN1C(=O)OC(C)(C)C)CC2(F)F]

Under nitrogen, to a solution of 5-(tert-butyl) 6-methyl (3S,6S)-1,1-difluoro-5-azaspiro[2.4]heptane-5,6-dicarboxylate (1.14 g, 3.91 mmol) (from the faster peak of previous step) in tetrahydrofuran (25 mL) was added HMPA (910 mg, 5.09 mmol) at room temperature. Then LiHMDS (5.00 mL, 5.00 mmol) was added at −40° C. The resulting solution was stirred for 0.5 h at −40° C. Then 1-bromo-2-chloroethane (2.80 g, 19.5 mmol) was added at −40° C. The reaction was wormed to room temperature and stirred 2 hours. The reaction was quenched with aq. NH₄Cl, extracted with EtOAc. The combined organic layers were dried over anhydrous sodium sulfate and concentrated under vacuum. The residue was purified by flash chromatography on silica gel (gradient: 0-30% EtOAc/petroleum ether) to afford 420 mg (30% yield) of the title compound as a yellow oil (intermediate 3a) and 530 mg mixture of starting material and the another isomer (compound 3b). The 530 mg mixture was re-purified by C18 column (solvent gradient: 0-100% ACN in water (10 M NH₄HCO₃)) to yield 117 mg (8% yield) of the title compound as a yellow oil (intermediate 3b). LC-MS: (ESI, m/z): [M+H]⁺=354.

intermediate 3a: ¹H NMR (400 MHz, DMSO-d₆) δ 3.91-3.75 (m, 1H), 3.71-3.44 (m, 6H), 2.61-2.52 (m, 2H), 2.44-2.31 (m, 1H), 2.13-2.03 (m, 1H), 1.75-1.52 (m, 2H), 1.37 (d, J=19.6 Hz, 9H).

intermediate 3b: ¹H NMR (300 MHz, DMSO-d₆) δ 3.81-3.62 (m, 4H), 3.61-3.44 (m, 3H), 2.46-2.17 (m, 4H), 1.81-1.51 (m, 2H), 1.37 (d, J=13.8 Hz, 9H).

Under nitrogen, to a solution of 5-(tert-butyl) 6-methyl (3R,6S)-1,1-difluoro-5-azaspiro[2.4]heptane-5,6-dicarboxylate (600 mg, 2.06 mmol) (from the slower peak of previous step) in tetrahydrofuran (15 mL) was added HMPA (479 mg, 2.67 mmol) at room temperature. Then the reaction system was cooled to −40° C. and LiHMDS (2.67 mL, 2.67 mmol) was added. The resulting solution was stirred for 0.5 h at −40° C. Then 1-bromo-2-chloroethane (1.48 g, 10.3 mmol) was added at −40° C. The reaction was warmed to room temperature and stirred 2 hours. The reaction was quenched with aq. NH₄Cl, extracted with EtOAc. The combined organic layers were dried over anhydrous sodium sulfate and concentrated under vacuum. The residue was purified by flash chromatography on silica gel (gradient: 0-30% EtOAc/petroleum ether) to afford 200 mg (27% yield) of the title compound as a yellow oil (intermediate 3c) and 200 mg mixture of starting material and the another isomer (compound 3d). The 200 mg mixture was re-purified by C18 column (solvent gradient: 0-100% ACN in water (10 M NH₄HCO₃)) to yield 63 mg (8% yield) of the title compound as a yellow oil (intermediate 3d). LC-MS: (ESI, m/z): [M+H]⁺=354.

intermediate 3c: ¹H NMR (400 MHz, DMSO-d₆) δ 3.91-3.75 (m, 1H), 3.71-3.44 (m, 6H), 2.61-2.52 (m, 2H), 2.44-2.31 (m, 1H), 2.13-2.03 (m, 1H), 1.75-1.52 (m, 2H), 1.37 (d, J=19.6 Hz, 9H)

intermediate 3d: ¹H NMR (300 MHz, DMSO-d₆) δ 3.81-3.62 (m, 4H), 3.61-3.44 (m, 3H), 2.46-2.17 (m, 4H), 1.81-1.51 (m, 2H), 1.37 (d, J=13.8 Hz, 9H)

### Step 3: Methyl (3S)-6-(2-chloroethyl)-1,1-difluoro-5-azaspiro[2.4]heptane-6-carboxylate (two single unknown isomers) and methyl (3R)-6-(2-chloroethyl)-1,1-difluoro-5-azaspiro[2.4]heptane-6-carboxylate (two single unknown isomers)

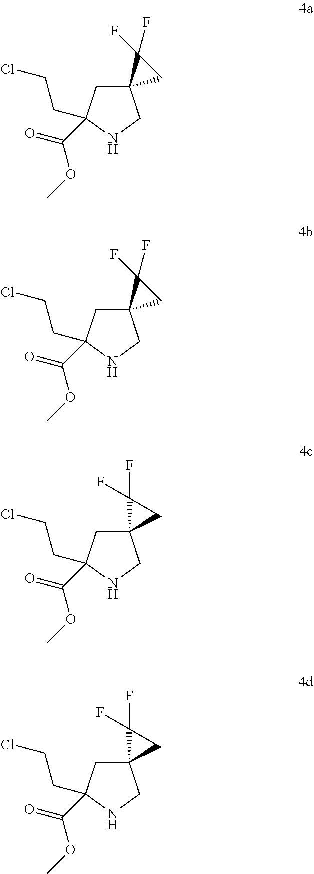 [SMILES: COC(=O)C1(CCCl)C[C@@]2(CN1)CC2(F)F ; COC(=O)C1(CCCl)C[C@@]2(CN1)CC2(F)F ; COC(=O)C1(CCCl)C[C@]2(CN1)CC2(F)F ; COC(=O)C1(CCCl)C[C@]2(CN1)CC2(F)F]

A solution of 5-(tert-butyl) 6-methyl (3S)-6-(2-chloroethyl)-1,1-difluoro-5-azaspiro[2.4]heptane-5,6-dicarboxylate (260 mg, 0.736 mmol) (compound 3a) and TFA (0.6 mL) in dichloromethane (3 mL) was stirred at room temperature for 1 h. The solvent was concentrated under vacuum to afford the crude product and the crude product was used for the next step without further purification. LC-MS: (ESI, m/z): [M+H]⁺=254.

Analogous to the method described above, the other 3 isomers were prepared from relevant Boc starting materials.

### Step 4: Methyl (3S)-2′,2′-difluoro-1-azaspiro[bicyclo[3.2.0]heptane-3,1′-cyclopropane]-5-carboxylate (two single unknown atropisomers) and methyl (3R)-2′,2′-difluoro-1-azaspiro[bicyclo[3.2.0]heptane-3,1′-cyclopropane]-5-carboxylate (two single unknown atropisomers)

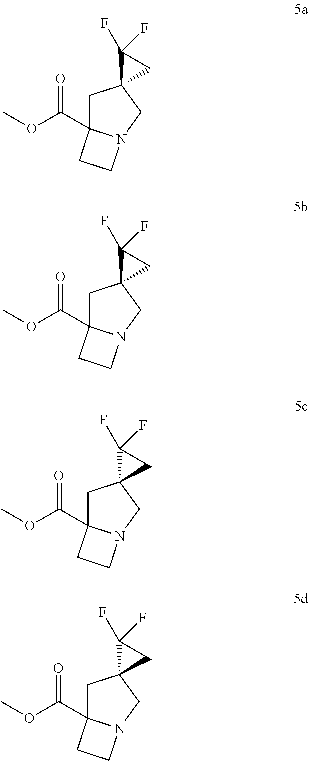

A solution of methyl (3S)-6-(2-chloroethyl)-1,1-difluoro-5-azaspiro[2.4]heptane-6-carboxylate (186 mg, 0.735 mmol) and Et₃N (371 mg, 3.67 mmol) in acetonitrile (10 mL) was stirred at 85° C. for 4 hours. The solvent was concentrated under vacuum. The residue was purified by flash chromatography on silica gel (gradient: 0-10% MeCOH/DCM) to yield 90 mg (56% yield, intermediate 5a) as a yellow oil. LC-MS: (ESI, m/z): [M+H]⁺=218. ¹H NMR (300 MHz, DMSO-d₆) δ 3.64 (s, 3H), 3.42-3.33 (m, 1H), 3.27-3.16 (m, 1H), 3.04 (d, J=12.6 Hz, 1H), 2.76-2.67 (m, 1H), 2.65-2.54 (m, 1H), 2.40-2.31 (m, 1H), 2.32-2.21 (m, 2H), 1.85-1.75 (m, 2H).

Analogous to the method described above, the other 3 isomers were prepared from relevant starting materials.

intermediate 5b: ¹H NMR (400 MHz, DMSO-d₆) δ 3.67 (s, 3H), 3.42-3.33 (m, 1H), 3.09-2.97 (m, 1H), 2.79 (d, J=8.8 Hz, 1H), 2.71-2.58 (m, 2H), 2.33 (d, J=7.6 Hz, 1H), 2.19-2.05 (m, 2H), 1.73-1.65 (m, 1H), 1.62-1.52 (m, 1H).

intermediate 5c: ¹H NMR (300 MHz, DMSO-d₆) δ 3.64 (s, 3H), 3.42-3.33 (m, 1H), 3.27-3.16 (m, 1H), 3.04 (d, J=12.6 Hz, 1H), 2.76-2.67 (m, 1H), 2.65-2.54 (m, 1H), 2.40-2.31 (m, 1H), 2.32-2.21 (m, 2H), 1.85-1.75 (m, 2H).

intermediate 5d: ¹H NMR (400 MHz, Chloroform-d) δ 3.87-3.77 (m, 3H), 3.75-3.65 (m, 1H), 3.30-3.20 (m, 1H), 3.12-3.00 (m, 1H), 2.87-2.76 (m, 2H), 2.49-2.37 (m, 1H), 2.36-2.21 (m, 2H), 1.60-1.39 (m, 2H).

### Step 5: ((3S)-2′,2′-Difluoro-1-azaspiro[bicyclo[3.2.0]heptane-3,1′-cyclopropan]-5-yl)methanol (two single unknown atropisomers) and ((3R)-2′,2′-difluoro-1-azaspiro[bicyclo[3.2.0]heptane-3,1′-cyclopropan]-5-yl)methanol (two single unknown atropisomers)

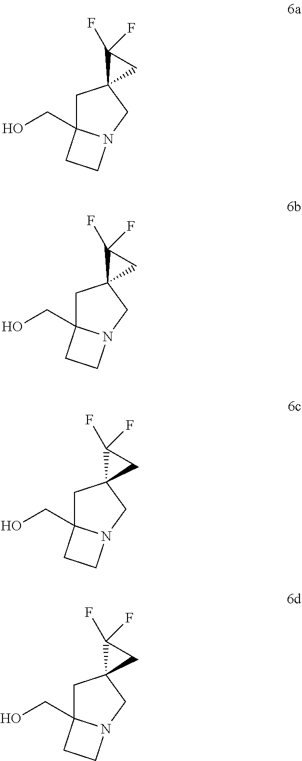

Under nitrogen, to a solution of methyl (3S)-2′,2′-difluoro-1-azaspiro[bicyclo[3.2.0]heptane-3,1′-cyclopropane]-5-carboxylate (97.0 mg, 0.450 mmol) in tetrahydrofuran (7 mL) was added LiAlH₄ (0.9 mL, 0.900 mmol) at 0° C. The resulting solution was stirred for 0.5 h at room temperature. The reaction was quenched with Na₂SO₄·10H₂O. After filtration, the filtrate was concentrated under reduced pressure to afford the product (100 mg, crude, intermediate 6a) as a yellow oil. The crude product was used for the next step without further purification. LC-MS: (ESI, m/z): [M+H]⁺=190. ¹H NMR (400 MHz, DMSO-d₆) δ 3.42-3.33 (m, 1H), 3.31-3.22 (m, 2H), 3.21-3.10 (m, 2H), 3.04-2.91 (m, 1H), 2.61-2.55 (m, 1H), 2.25-2.11 (m, 2H), 2.10-2.01 (m, 1H), 1.91-1.81 (m, 1H), 1.78-1.70 (m, 2H).

Analogous to the method described above, the other 3 isomers were prepared from relevant starting materials.

intermediate 6b: ¹H NMR (400 MHz, DMSO-d₆) δ 4.75-4.61 (m, 1H), 3.40-3.20 (m, 5H), 2.95-2.81 (m, 1H), 2.80-2.70 (m, 1H), 2.25-2.13 (m, 1H), 2.07-1.95 (m, 1H), 1.92-1.83 (m, 1H), 1.61-1.50 (m, 1H), 1.49-1.40 (m, 1H).

intermediate 6c: ¹H NMR (400 MHz, DMSO-d₆) δ 3.42-3.33 (m, 1H), 3.31-3.22 (m, 2H), 3.21-3.10 (m, 2H), 3.04-2.91 (m, 1H), 2.61-2.55 (m, 1H), 2.25-2.11 (m, 2H), 2.10-2.01 (m, 1H), 1.91-1.81 (m, 1H), 1.78-1.70 (m, 2H).

intermediate 6d: didn't run HNMR due to small amount

### Intermediate 35: (3,3-Difluoro-1-azabicyclo[3.2.0]heptan-5-yl)methanol

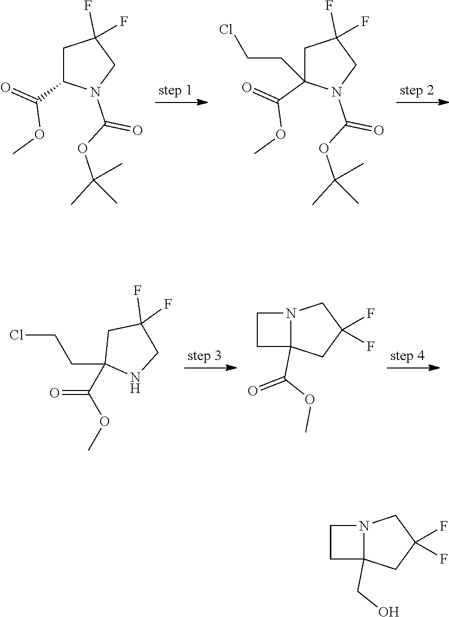 [SMILES: COC(=O)[C@@H]1CC(F)(F)CN1C(=O)OC(C)(C)C ; COC(=O)C1(CCCl)CC(F)(F)CN1C(=O)OC(C)(C)C ; COC(=O)C1(CCCl)CC(F)(F)CN1 ; COC(=O)C12CCN1CC(F)(F)C2 ; OCC12CCN1CC(F)(F)C2]

### Step 1. 1-(tert-Butyl) 2-methyl 2-(2-chloroethyl)-4,4-difluoropyrrolidine-1,2-dicarboxylate

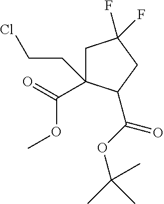 [SMILES: COC(=O)C1(CCCl)CC(F)(F)CC1C(=O)OC(C)(C)C]

Under nitrogen, to a solution of 1-tert-butyl 2-methyl (2S)-4,4-difluoropyrrolidine-1,2-dicarboxylate (2.0 g, 7.54 mmol) in tetrahydrofuran (30 mL) was added LiHMDS (10 mL, 10 mmol) at −78° C. and the mixture was stirred at −78° C. for 0.5 hours. Then 1-chloro-2-iodoethane (2.87 g, 15.1 mmol) was added and stirred at room temperature for 1 hour. The reaction was quenched by NH₄Cl (aq.), extracted with EtOAc. The combined organic layers were dried over NaSO₄ and concentrated under vacuum. The residue was purified by flash chromatography on silica gel eluting with EtOAc/petroleum ether (0-60%) to afford the title compound 900 mg (36.4% yield) as a yellow solid. LC-MS: (ESI, m/z): [M+H]⁺=328

### Step 2. Methyl 2-(2-chloroethyl)-4,4-difluoropyrrolidine-2-carboxylate

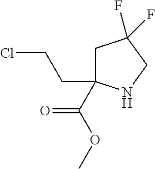

Under nitrogen, to a solution of 1-(tert-butyl) 2-methyl 2-(2-chloroethyl)-4,4-difluoropyrrolidine-1,2-dicarboxylatee (470 mg, 1.43 mmol) in dichloromethane (5 mL) was added TFA (1 mL) at room temperature. The resulting solution was stirred at room temperature for 1 hour. The solvent was concentrated under vacuum to afford the title compound 300 mg (crude) as a yellow oil which was used for next step without further purification. LC-MS: (ESI, m/z): [M+H]⁺=228

### Step 3. Methyl 3,3-difluoro-1-azabicyclo[3.2.0]heptane-5-carboxylate

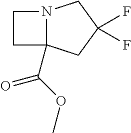

Under nitrogen, to a solution of methyl 2-(2-chloroethyl)-4,4-difluoro-pyrrolidine-2-carboxylate (300 mg, 1.32 mmol) in acetonitrile (5 mL) was added Et₃N (1 mL) at room temperature, and the mixture was stirred at 85° C. for 12 hours. The solvent was concentrated under vacuum. The residue was purified by flash chromatography on silica gel eluting with DCM/MeOH (0-6%) to afford the title compound 150 mg (59.5% yield) as a yellow solid. LC-MS: (ESI, m/z): [M+H]⁺=192

### Step 4. (3,3-Difluoro-1-azabicyclo[3.2.0]heptan-5-yl)methanol

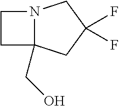 [SMILES: OCC12CCN1CC(F)(F)C2]

Under nitrogen, to a solution of methyl 3,3-difluoro-1-azabicyclo[3.2.0]heptane-5-carboxylate (150 mg, 0.780 mmol) in tetrahydrofuran (3 mL) was added LiAlH₄ (2.4 mL, 2.4 mmol, 1M in THF) at 0° C. The solution was stirred at 0° C. for 0.5 hours. The solvent was remove by blowing nitrogen to afford 110 mg (crude) the title compound as a yellow oil which was used for next reaction without further purification. LC-MS: (ESI, m/z): [M+H]⁺=164. purification.

### Intermediate 36: ((7a'S)-2,2-Difluorodihydro-1′H,3′H-spiro[cyclopropane-1,2′-pyrrolizin]-7a′(5′H)-yl)methanol

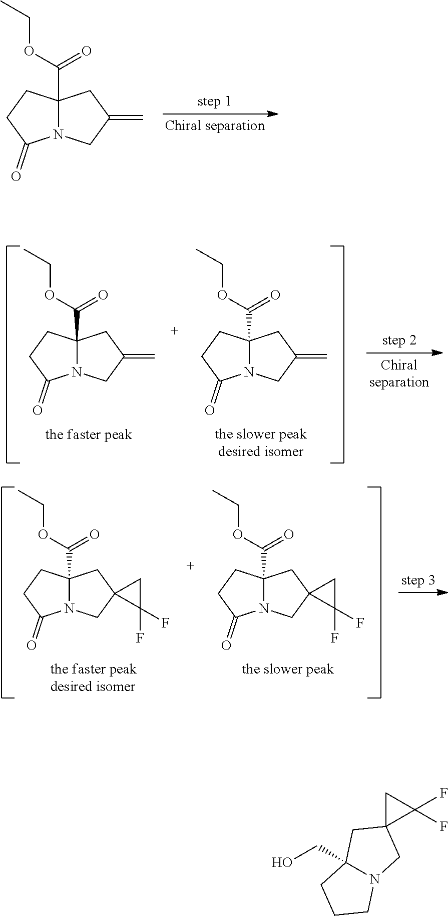

### Step 1: Ethyl (R)-2-methylene-5-oxotetrahydro-1H-pyrrolizine-7a(5H)-carboxylate & Ethyl (S)-2-methylene-5-oxotetrahydro-1H-pyrrolizine-7a(5H)-carboxylate

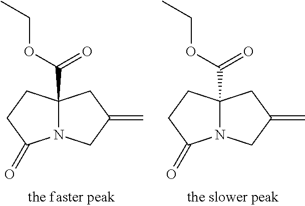

Ethyl 2-methylene-5-oxotetrahydro-1H-pyrrolizine-7a(5H)-carboxylate (19.9 g, was separated by Chiral SFC (Column: CHIRALPAK IH, 50*250 mm; Mobile Phase A: CO2, Mobile Phase B: EtOH; Flow rate: 150 mL/min; Gradient:26% B; 220 nm; RT1:4.8; RT2:6.43; Injection Volumn:1.8 ml; Number Of Runs: 122) to afford ethyl (R)-2-methylene-5-oxotetrahydro-1H-pyrrolizine-7a(5H)-carboxylate (7.61 g, the faster peak) and ethyl (S)-2-methylene-5-oxotetrahydro-1H-pyrrolizine-7a(5H)-carboxylate (7.29 g, the slower peak). LC-MS: (ESI, m/z): [M+H]⁺=210. ¹H NMR (400 MHz, Chloroform-d) δ5.12-5.00 (m, 2H), 4.32-4.28 (m, 1H), 4.21 (q, J=7.1 Hz, 2H), 3.73 (d, J=15.7 Hz, 1H), 3.06 (d, J=15.7 Hz, 1H), 2.85-2.72 (m, 1H), 2.66-2.57 (m, 1H), 2.53-2.41 (m, 2H), 2.19-2.08 (m, 1H), 1.28 (t, J=7.1 Hz, 3H). The HNMR of two isomers are same.

### Step 2: Ethyl (7a'S)-2,2-difluoro-5′-oxodihydro-1′H,3′H-spiro[cyclopropane-1,2′-pyrrolizine]-7a′(5′H)-carboxylate

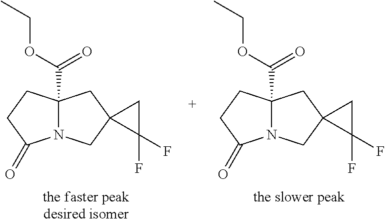 [SMILES: CCOC(=O)[C@@]12CCC(=O)N1CC1(CC1(F)F)C2 ; CCOC(=O)[C@@]12CCC(=O)N1CC1(CC1(F)F)C2]

Under nitrogen, to a solution of ethyl (S)-2-methylene-5-oxotetrahydro-1H-pyrrolizine-7a(5H)-carboxylate (200 mg, 0.960 mmol) and NaI (71.7 mg, 0.480 mmol) in THF (5 mL) was added TMSCF₃ (476 mg, 3.35 mmol) at room temperature. The resulting solution was stirred for 2.5 h at 65° C. The solution was diluted with DCM, washed with sodium thiosulfate solution and dried over with Na₂SO₄. The organic layer was concentrated under vacuum. The residue was purified by flash chromatography on silica gel (gradient: 0%-35% ethyl acetate/petroleum ether) to afford the faster peak 95.0 mg (38.3% yield) and (gradient: 35%-90% ethyl acetate/petroleum ether) to afford the slower peak 108 mg (43.6% yield) as a yellow solid. LC-MS: (ESI, m/z): [M+H]⁺=260. The faster peak: ¹H NMR (300 MHz, DMSO-d₆, ppm) δ 4.25-4.19 (m, 2H), 3.80-3.70 (m, 1H), 3.04 (d, J=12.1, 3.5 Hz, 1H), 2.62-2.58 (m, 1H), 2.51-2.12 (m, 5H), 1.60 (t, J=9.3 Hz, 2H), 1.24 (t, J=7.1 Hz, 3H). The slower peak: ¹H NMR (300 MHz, DMSO-d₆, ppm) δ 4.24-4.08 (m, 2H), 3.61 (d, J=11.7, 2.7 Hz, 1H), 3.11 (d, J=11.6 Hz, 1H), 2.72-2.54 (m, 1H), 2.44-2.13 (m, 5H), 1.79-1.58 (m, 2H), 1.22 (t, J=7.1 Hz, 3H).

### Step 2: ((7a'S)-2,2-difluorodihydro-1′H,3′H-spiro[cyclopropane-1,2′-pyrrolizin]-7a′(5′H)-yl)methanol

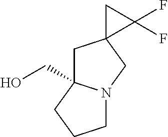 [SMILES: OC[C@@]12CCCN1CC1(CC1(F)F)C2]

Under nitrogen, to a solution of ethyl (7a'S)-2,2-difluoro-5′-oxodihydro-1′H,3′H-spiro[cyclopropane-1,2′-pyrrolizine]-7a′(5′H)-carboxylate (95.0 mg, 0.370 mmol, the faster peak of previous step) in THF (2.5 mL) was added LiAlH₄ (1.1 mL, 1 M in THF). The solution was stirred at 65° C. for 1 hour. The reaction was cooled to room temperature and quenched with Na₂SO₄ 10H₂O. After filtration, the filtrate was concentrated under reduced pressure to afford 42.0 mg (56.4% yield) of the title compound as a yellow oil. LC-MS: (ESI, m/z): [M+H]⁺=204.

### Intermediate 37: 7-(6-(Bis(4-methoxybenzyl)amino)-4-methyl-3-(trifluoromethyl)pyridin-2-yl)-2,6-dichloro-5,8-difluoroquinazolin-4 (3H)-one

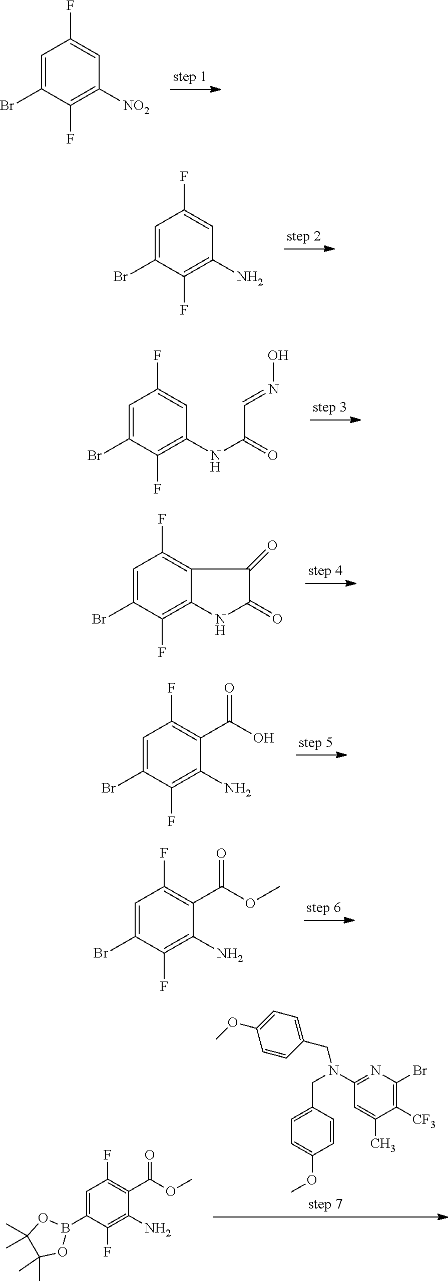

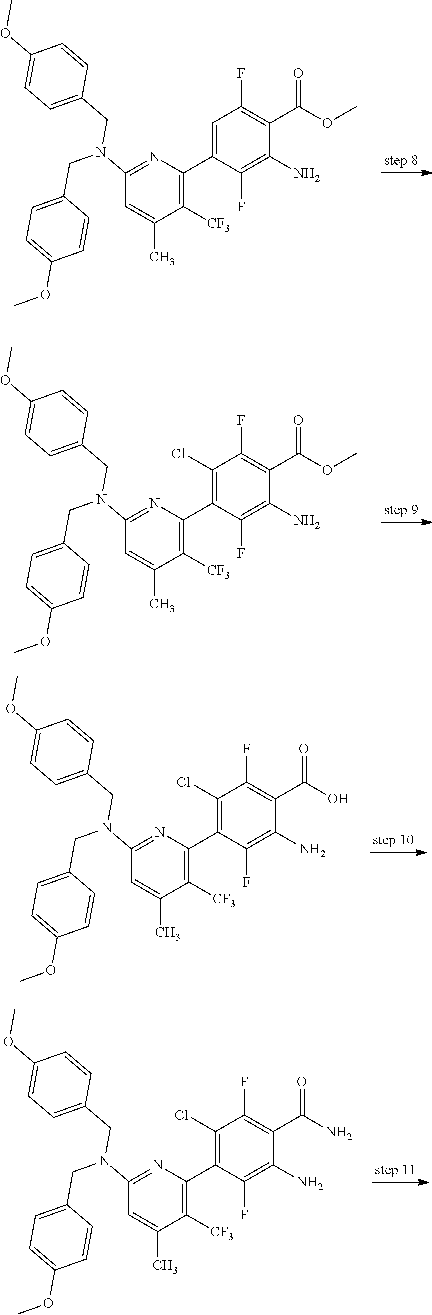

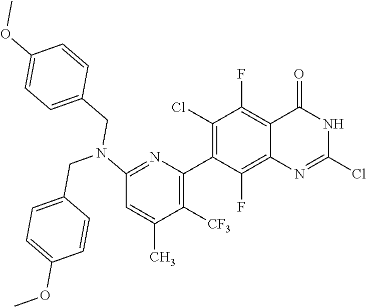 [SMILES: COc1ccc(CN(Cc2ccc(OC)cc2)c2cc(C)c(C(F)(F)F)c(-c3c(Cl)c(F)c4c(=O)[nH]c(Cl)nc4c3F)n2)cc1]

### Step 1: 3-Bromo-2,5-difluoroaniline

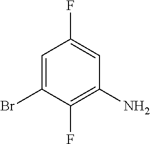

To a solution of 1-bromo-2,5-difluoro-3-nitrobenzene (40.0 g, 168 mmol), and iron powder (28.4 g, 506 mmol) in water (10 mL) was added concentrated hydrochloric acid (40 mL, 36%) at room temperature. The solution was heated to 100° C. for 1 h. The reaction system was cooled to room temperature. The solid was filtered off and washed with EtOAc. The combined filtrates were concentrated under reduced pressure to afford the title compound (34.3 g, crude) as a brown oil, LC-MS: (ESI, m/z): [M+H]⁺=208. The crude was used for next step without further purification.

### Step 2: N-(3-Bromo-2,5-difluorophenyl)-2-(hydroxyimino)acetamide

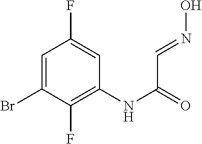 [SMILES: O=C(/C=N/O)Nc1cc(F)cc(Br)c1F]

To a solution of 2,2,2-trichloroethane-1,1-diol (40.9 g, 247 mmol), Na₂SO₄ (187 g, 1.32 mol) and NH₂OH·HCl (39.8 g, 577 mmol) in water (680 mL) was added a solution of 3-bromo-2,5-difluoroaniline (34.3 g, 165 mmol) in ethanol (100 mL), hydrochloric acid (12.5 mL, 36%) and water (50 ml). The resulting solution was heated at 60° C. for 3 h. The reaction system was cooled to room temperature and filtered. The solid was collected, washed with water (500 mL) and dried in oven to afford the title compound (32.8 g, crude) as a light brown solid which was used for next step without further purification. LC-MS: (ESI, m/z): [M+H]⁺=279.

### Step 3: 6-Bromo-4,7-difluoroindoline-2,3-dione

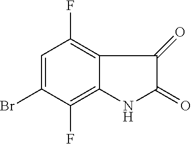 [SMILES: O=C1Nc2c(F)c(Br)cc(F)c2C1=O]

A solution of N-(3-bromo-2,5-difluorophenyl)-2-(hydroxyimino)acetamide (32.8 g, 118 mmol) in H₂SO₄ (160 mL, 98%) was heated at 90° C. for 1 h. The reaction mixture was cooled to room temperature and added to ice water slowly. The precipitate was collected by filtration, washed with water and dried in oven to afford the title compound (28.1 g, crude) as a brown solid which was used for next step without further purification. LC-MS: (ESI, m/z): [M+H]⁺=262.

### Step 4: 2-Amino-4-bromo-3,6-difluorobenzoic acid

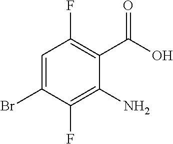 [SMILES: Nc1c(F)c(Br)cc(F)c1C(=O)O]

A solution of 6-bromo-4,7-difluoroindoline-2,3-dione (28.1 g, 107 mmol) in NaOH (537 mL, 2M in water) and H₂O₂ (53.7 mL, 30%) was stirred at room temperature for 16 h. The mixture was poured into ice water and adjusted to pH=2 with conc. HCl. The solid was collected by filtration and washed with water. The crude product was purified by reverse phase chromatography (gradient: 0-60% acetonitrile in water (0.1% formic acid)) to afford the title compound (13.4 g, 49.4% yield) as a light brown solid. LC-MS: (ESI, m/z): [M+H]⁺=252.

### Step 5: Methyl 2-amino-4-bromo-3,6-difluorobenzoate

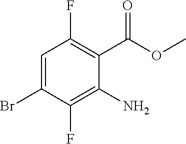

To a solution of 2-amino-4-bromo-3,6-difluoro-benzoic acid (50.0 g, 198 mmol) in dichloromethane (75 mL) and methyl alcohol (75 mL) was added TMSCHN₂ (100 mL, 2.0 mol/L in hexane) at 0° C. The resulting solution was stirred at room temperature for 2 h. The solvent was concentrated under vacuum to yield 52 g (crude) of the title compound as a yellow solid. The crude product was used for next step without further purification. LC-MS: (ESI, m/z): [M+H]⁺=266.

### Step 6: Methyl 2-amino-3,6-difluoro-4-(4,4,5,5-tetramethyl-1,3,2-dioxaborolan-2-yl)benzoate

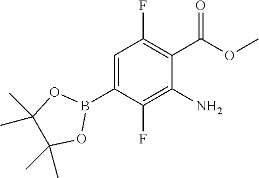

Under nitrogen, a solution of methyl 2-amino-4-bromo-3,6-difluoro-benzoate (25.0 g, 93.9 mmol), Pin₂B₂ (35.8 g, 141 mmol), KOAc (27.6 g, 282 mmol) and PdCl₂(dppf) (6.88 g, 9.40 mmol) in 1,4-dioxane (300 mL) was stirred at 90° C. for 2 h. The solution was cooled to room temperature, diluted with water and extracted with ethyl acetate. The combined organic layers were washed with brine, dried over anhydrous sodium sulfate and concentrated under vacuum to afford 35 g (crude) as brown solid. The crude product was used for next step without further purification. LC-MS: (ESI, m/z): [M+H]⁺=314.

### Step 7: Methyl 2-amino-4-(6-(bis(4-methoxybenzyl)amino)-4-methyl-3-(trifluoromethyl)pyridin-2-yl)-3,6-difluorobenzoate

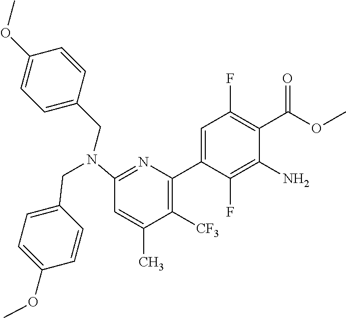

Under nitrogen, a solution of methyl 2-amino-3,6-difluoro-4-(4,4,5,5-tetramethyl-1,3,2-dioxaborolan-2-yl)benzoate (58.0 g, 185 mmol), 6-bromo-N,N-bis[(4-methoxyphenyl)methyl]-4-methyl-5-(trifluoromethyl)pyridin-2-amine (73.4 g, 148 mmol, intermediate 4), KF (20.4 g, 352 mmol) and Pd(PPh₃)₂Cl₂ (13.0 g, 18.5 mmol) in acetonitrile (500 mL) and water (100 mL) was stirred at 70° C. for 2 h. The reaction mixture was cooled to room temperature, diluted with water and extracted with ethyl acetate. The combined organic layers were concentrated under vacuum to afford 75 g (crude) as brown solid. The crude product was used for next step without purification. LC-MS: (ESI, m/z): [M+H]⁺=602.

### Step 8: Methyl 2-amino-4-(6-(bis(4-methoxybenzyl)amino)-4-methyl-3-(trifluoromethyl)pyridin-2-yl)-5-chloro-3,6-difluorobenzoate

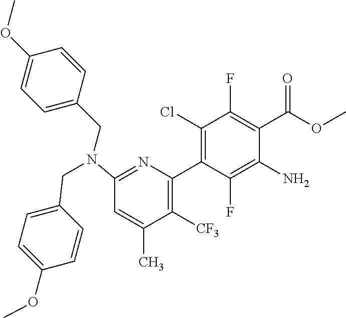

A solution of methyl 2-amino-4-[6-[bis[(4-methoxyphenyl)methyl]amino]-4-methyl-3-(trifluoromethyl)-2-pyridyl]-3,6-difluoro-benzoate (15.0 g, 24.9 mmol) and NCS (4.99 g, 37.4 mmol) in 1-methyl-2-pyrrolidinone (350 mL) was stirred at 25° C. for 2 h. The reaction mixture was diluted with water, extracted with ethyl acetate. The combined organic layers were washed with water, dried over Na₂SO₄ and concentrated under vacuum to yield 15.8 g (crude) of the title compound as a brown solid. The crude product was used for next step without further purification. LC-MS: (ESI, m/z): [M+H]⁺=636.

### Step 9: 2-Amino-4-(6-(bis(4-methoxybenzyl)amino)-4-methyl-3-(trifluoromethyl)pyridin-2-yl)-5-chloro-3,6-difluorobenzoic acid

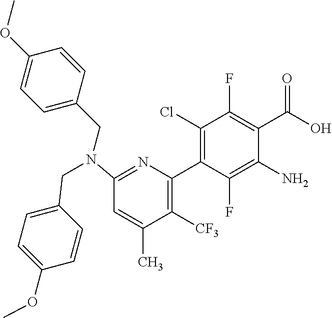 [SMILES: COc1ccc(CN(Cc2ccc(OC)cc2)c2cc(C)c(C(F)(F)F)c(-c3c(F)c(N)c(C(=O)O)c(F)c3Cl)n2)cc1]

To a solution of methyl 2-amino-4-[6-[bis[(4-methoxyphenyl)methyl]amino]-4-methyl-3-(trifluoromethyl)-2-pyridyl]-5-chloro-3,6-difluoro-benzoate (16.0 g, 25.2 mmol) in tetrahydrofuran (150 mL),water (50 mL) and methyl alcohol (50 mL) was added NaOH (15.1 g, 377 mmol). The solution was stirred at 50° C. for 3 h. The reaction system was cooled to room temperature and adjusted the pH to ˜4 with citric acid. The reaction mixture was diluted with water, extracted with ethyl acetate. The combined organic layers were dried over Na₂SO₄ and concentrated under vacuum to yield 15.8 g (crude) of the title compound as a brown solid. The crude product was used for next step without further purification. LC-MS: (ESI, m/z): [M+H]⁺=622.

### Step 10: 2-Amino-4-(6-(bis(4-methoxybenzyl)amino)-4-methyl-3-(trifluoromethyl)pyridin-2-yl)-5-chloro-3,6-difluorobenzamide

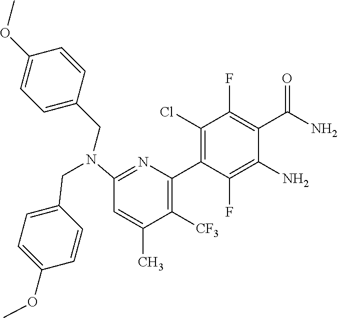 [SMILES: COc1ccc(CN(Cc2ccc(OC)cc2)c2cc(C)c(C(F)(F)F)c(-c3c(F)c(N)c(C(N)=O)c(F)c3Cl)n2)cc1]

A solution of 2-amino-4-[6-[bis[(4-methoxyphenyl)methyl]amino]-4-methyl-3-(trifluoromethyl)-2-pyridyl]-5-chloro-3,6-difluoro-benzoic acid (14.5 g, 23.3 mmol), NH₄Cl (3.74 g, 69.9 mmol), DIPEA (9.04 g, 69.9 mmol) and HATU (17.7 g, 46.6 mmol) in DMF (150 mL) was stirred at room temperature for 2 hours. The reaction solution was poured into water, the solids were collected by filtration and washed with water to afford 13.5 g (crude) of the title compound as a brown solid. The crude product was used for the next step without further purification. LC-MS: (ESI, m/z): [M+H]⁺=621.

### Step 11: 7-(6-(Bis(4-methoxybenzyl)amino)-4-methyl-3-(trifluoromethyl)pyridin-2-yl)-2,6-dichloro-5,8-difluoroquinazolin-4 (3H)-one

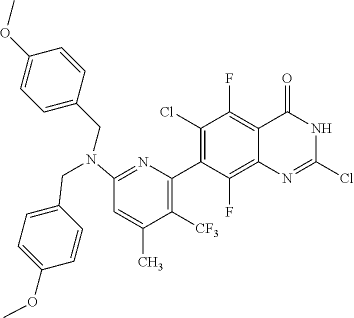 [SMILES: COc1ccc(CN(Cc2ccc(OC)cc2)c2cc(C)c(C(F)(F)F)c(-c3c(Cl)c(F)c4c(=O)[nH]c(Cl)nc4c3F)n2)cc1]

A solution of 2-amino-4-[6-[bis[(4-methoxyphenyl)methyl]amino]-4-methyl-3-(trifluoromethyl)-2-pyridyl]-5-chloro-3,6-difluoro-benzamide (30.0 g, 48.3 mmol) and thiophosgene (7.74 mL, 101 mmol) in 1,4-dioxane (250 mL) was stirred at 105° C. for 1 hour. The reaction system was cooled to room temperature and poured into saturated NaHCO₃. The resulting solution was extracted with ethyl acetate. The combined organic layers were dried over Na₂SO₄ and concentrated under vacuum. The residue was purified by flash chromatography on silica gel (gradient: 0-8% EtOAc/DCM) to yield 11 g (34.2% yield) of the title compound as a yellow solid. LC-MS: (ESI, m/z): [M+H]⁺=665. ¹H NMR (300 MHz, DMSO-d₆, ppm): δ 7.25-7.06 (m, 4H), 6.88 (d, J=8.0 Hz, 5H), 4.88-4.49 (m, 4H), 3.35 (s, 6H), 2.40 (d, J=2.3 Hz, 3H).

### Intermediate 38: tert-Butyl (1R,2S,5R)-2-(hydroxymethyl)-3,6-diazabicyclo[3.2.2]nonane-6-carboxylate

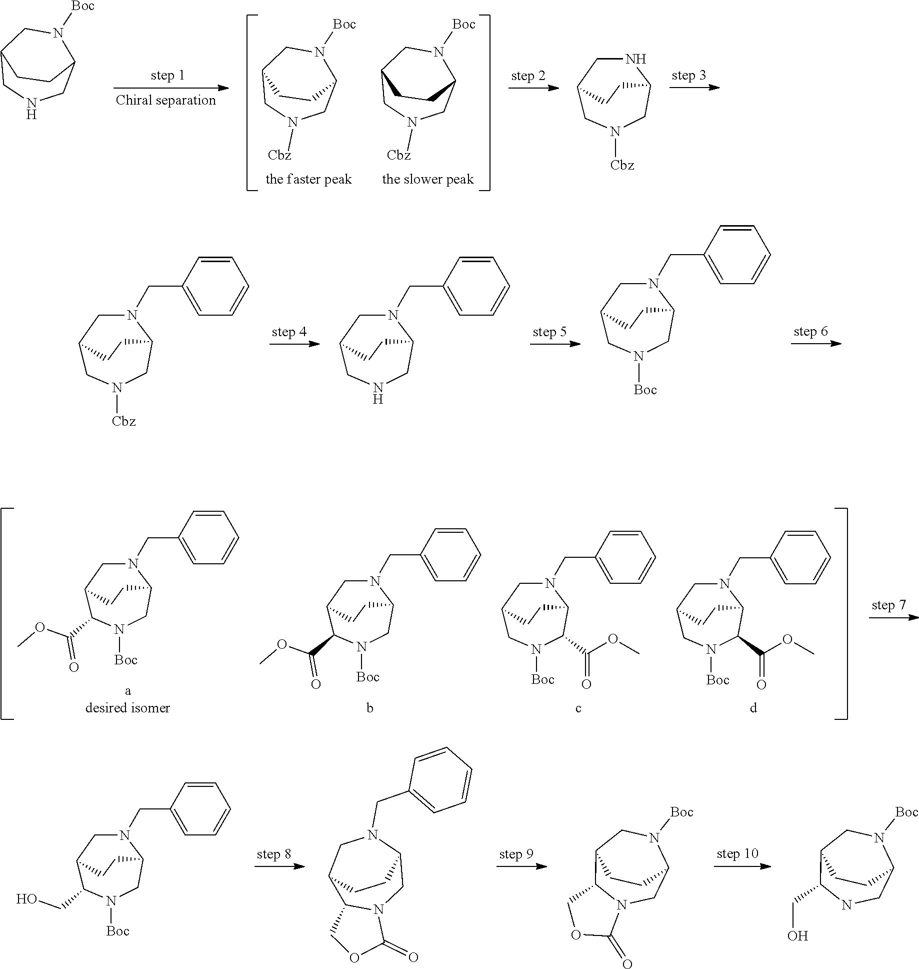

### Step 1: 3-Benzyl 6-(tert-butyl) (1 S,5R)-3,6-diazabicyclo[3.2.2]nonane-3,6-dicarboxylate

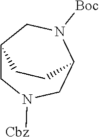 [SMILES: CC(C)(C)OC(=O)N1C[C@H]2CC[C@@H]1CN(C(=O)OCc1ccccc1)C2]

Under nitrogen, a solution of tert-butyl 3,6-diazabicyclo[3.2.2]nonane-6-carboxylate (500 mg, 2.21 mmol), CbzCl (492 mg, 2.88 mmol) and DIPEA (1.42 g, 11.1 mmol) in dichloromethane (10 mL) was stirred at room temperature for 1 h. The reaction mixture was diluted with DCM, washed with water. The organic layer was dried over anhydrous sodium sulfate and concentrated under vacuum. The residue was purified by flash chromatography on silica gel (gradient: 0-100% EtOAc/petroleum ether) to afford 800 mg of the title compound as a colorless oil. The two enantiomers were separated by Prep-SFC with the following conditions: (Column: CHIRALPAK IG, 3*25 cm, 5 μm; Mobile Phase A:CO₂, Mobile Phase B:IPA (0.5% 2M NH₃-MeOH); Flow rate:70 mL/min; Gradient:30% B; Column Temperature: 35° C.; Back Pressure: 100 bar; 215 nm; RT1:6.86; RT2:7.89; Injection Volumn:1.5 ml; Number Of Runs:20) to yield 330 mg of faster peak and 340 mg slower peak as a white oil. The faster peak is the desired isomer. LC-MS: (ESI, m/z): [M+H]⁺=361.

### Step 2: Benzyl (1R,5R)-3,6-diazabicyclo[3.2.2]nonane-3-carboxylate

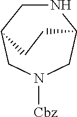 [SMILES: O=C(OCc1ccccc1)N1C[C@@H]2CC[C@H](C1)NC2]

A solution of 3-benzyl 6-(tert-butyl) (1S,5R)-3,6-diazabicyclo[3.2.2]nonane-3,6-dicarboxylate (10.0 g, 27.7 mmol) in dichloromethane (60 mL) and 4 M HCl/dioxane (20 mL) was stirred at room temperature for 2 h. The solvent was concentrated under vacuum to yield 11.2 g (crude) of the title compound as a yellow solid which was used for next step without further purification. LC-MS: (ESI, m/z): [M+H]⁺=261.

### Step 3: Benzyl (1R,5R)-6-benzyl-3,6-diazabicyclo[3.2.2]nonane-3-carboxylate

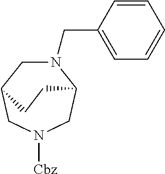 [SMILES: O=C(OCc1ccccc1)N1C[C@@H]2CC[C@H](C1)N(Cc1ccccc1)C2]

A solution of benzyl (1R,5R)-3,6-diazabicyclo[3.2.2]nonane-3-carboxylate (11.4 g, 43.7 mmol), benzyl bromide (8.99 g, 52.5 mmol) and DIPEA (11.3 g, 87.5 mmol) in N,N-dimethylformamide (50 mL) was stirred at 80° C. for 3 hours. The reaction was diluted with EtOAc and washed with water. The organic layers were dried over anhydrous Na₂SO₄ and concentrated under vacuum. The residue was purified by flash chromatography on silica gel (gradient: 0-40% EtOAc in petroleum ether) to afford 8.30 g of the title compound as a yellow oil. LC-MS: (ESI, m/z): [M+H]⁺=351

### Step 4: (1 S,5R)-6-Benzyl-3,6-diazabicyclo[3.2.2]nonane (2,2,2-trifluoroacetic acid salt)

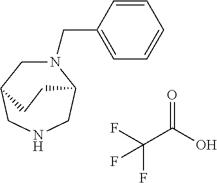

A solution of benzyl (1R,5R)-6-benzyl-3,6-diazabicyclo[3.2.2]nonane-3-carboxylate (8.30 g, 23.6 mmol) in TFA (60 mL) was stirred at 70° C. for 2 hours. The solvent was concentrated under vacuum to afford 5.80 g (crude) of the title compound as a yellow oil which was used for next step without further purification. LC-MS: (ESI, m/z): [M+H]⁺=217.

### Step 5: tert-Butyl (1R,5R)-6-benzyl-3,6-diazabicyclo[3.2.2]nonane-3-carboxylate

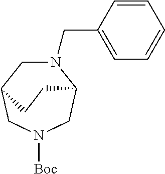 [SMILES: CC(C)(C)OC(=O)N1C[C@@H]2CC[C@H](C1)N(Cc1ccccc1)C2]

A solution of (1 S,5R)-6-benzyl-3,6-diazabicyclo[3.2.2]nonane (2,2,2-trifluoroacetic acid salt) (5.80 g, crude), Boc₂O (8.60 g, 39.4 mmol) and DIPEA (10.3 g, 79.8 mmol) in dichloromethane (120 mL) was stirred at room temperature for 2 h. The solvent was concentrated under vacuum and the resulting residue was purified by reverse phase chromatography (gradient: 0-100% acetonitrile in water (0.05% NH₄HCO₃)) to afford 6.30 g of the title compound as a yellow oil. LC-MS: (ESI, m/z): [M+H]⁺=317.

### Step 6: 3-(tert-Butyl) 2-methyl (1R,2S,5R)-6-benzyl-3,6-diazabicyclo[3.2.2]nonane-2,3-dicarboxylate

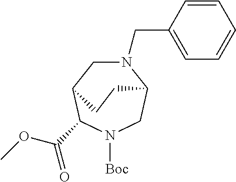 [SMILES: COC(=O)[C@@H]1[C@@H]2CC[C@H](CN1C(=O)OC(C)(C)C)N(Cc1ccccc1)C2]

Under nitrogen, to a solution of tert-butyl (1R,5R)-6-benzyl-3,6-diazabicyclo[3.2.2]nonane-3-carboxylate (6.09 g, 19.2 mmol) and TMEDA (2.72 g, 23.4 mmol) in diethyl ether (46 mL) was added s-BuLi (18.8 mL, 1.3 M in hexanes) at −78° C. The resulting solution was stirred at −78° C. for 1.5 h. Then methyl chloroformate (1.77 mL, 22.8 mmol) in diethyl ether (4.5 mL) was added and stirred at room temperature for 2 hours. The reaction was quenched with saturated NaHCO₃ solution. The resulting solution was extracted with EtOAc. The combined organic layers were dried over anhydrous Na₂SO₄ and concentrated under vacuum. The residue was purified by flash chromatography on silica gel (gradient: 0-50% EtOAc in petroleum ether) to yield 1.50 g (desire isomer (isomer a), the slower peak in column, contain trace b and d) and 2.6 g (the undesired isomer (isomer c), the faster peak in column, pure) as a yellow oil. LC-MS: (ESI, m/z): [M+H]⁺=375.

### Step 7: tert-Butyl (1R,2S,5R)-6-benzyl-2-(hydroxymethyl)-3,6-diazabicyclo[3.2.2]nonane-3-carboxylate

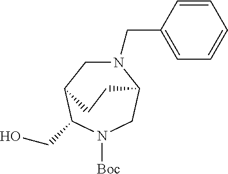 [SMILES: CC(C)(C)OC(=O)N1C[C@H]2CC[C@H](CN2Cc2ccccc2)[C@H]1CO]

Under nitrogen, to a solution of 3-(tert-butyl) 2-methyl (1R,2S,5R)-6-benzyl-3,6-diazabicyclo[3.2.2]nonane-2,3-dicarboxylate (580 mg, 1.54 mmol) was added LiAlH₄ (3.2 mL, 1 M in THF) at 0° C. The resulting solution was stirred at 0° C. for 2 h. The reaction was quenched with Na₂SO₄·10H₂O. After filtration, the filtrate was concentrated under reduced pressure. The residue was purified by flash chromatography on silica gel (gradient: 0-100% EtOAc in petroleum ether) to yield 520 mg of the title compound as a yellow oil. LC-MS: (ESI, m/z): [M+H]⁺=347. ¹H NMR (400 MHz, DMSO-d₆) δ 7.37-7.16 (m, 5H), 4.59 (d, J=42.8 Hz, 1H), 4.39-4.19 (m, 2H), 3.74-3.56 (m, 2H), 3.46 (dt, J=28.9, 10.3 Hz, 2H), 2.84 (s, 1H), 2.78-2.61 (m, 3H), 2.21 (s, 1H), 1.89 (dq, J=29.0, 15.0, 13.0 Hz, 2H), 1.46 (s, 11H).

### Step 8: (6R,9R,9aS)-11-Benzylhexahydro-1H,3H-6,9-(epiminomethano)oxazolo[3,4-a]azepin-3-one

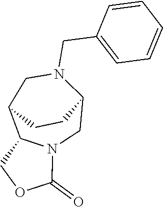 [SMILES: O=C1OC[C@@H]2[C@@H]3CC[C@H](CN12)N(Cc1ccccc1)C3]

Under nitrogen, to a solution of tert-butyl (1R,2S,5R)-6-benzyl-2-(hydroxymethyl)-3,6-diazabicyclo[3.2.2]nonane-3-carboxylate (480 mg, 1.39 mmol) in tetrahydrofuran (15 mL) was added NaH (112 mg, 2.82 mmol, 60% suspend in oil) at 0° C. The reaction was stirred at room temperature for 12 h. Aqueous NH₄Cl was added to quench the reaction. The solvent was concentrated under vacuum. The residue was purified by flash chromatography on silica gel (gradient: 0-60% EtOAc in petroleum ether) to yield 360 mg of the title compound as a white solid. LC-MS: (ESI, m/z): [M+H]⁺=273.

### Step 9: tert-Butyl (6R,9R,9aS)-3-oxohexahydro-1H,3H-6,9-(epiminomethano)oxazolo[3,4-a]azepine-11-carboxylate

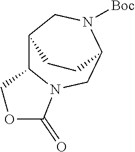 [SMILES: CC(C)(C)OC(=O)N1C[C@H]2CC[C@@H]1CN1C(=O)OC[C@H]21]

Under H₂ (3 atm), a solution of (6R,9R,9aS)-11-benzylhexahydro-1H,3H-6,9-(epiminomethano)oxazolo[3,4-a]azepin-3-one (330 mg, 1.20 mmol), 10% Pd/C (99.0 mg, dry) and Boc₂O (530 mg, 2.45 mmol) in methyl alcohol (70 mL) was stirred at room temperature for 1 hour. After filtration, the filtrate was concentrated under reduced pressure. The residue was purified by flash chromatography (gradient: 0-100% EtOAc in petroleum ether) to yield 350 mg of the title compound as a white solid. LC-MS: (ESI, m/z): [M+H]⁺=283.

### Step 10: tert-Butyl (1R,2S,5R)-2-(hydroxymethyl)-3,6-diazabicyclo[3.2.2]nonane-6-carboxylate

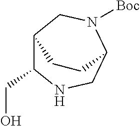 [SMILES: CC(C)(C)OC(=O)N1C[C@H]2CC[C@@H]1CN[C@@H]2CO]

A solution of tert-butyl (6R,9R,9aS)-3-oxohexahydro-1H,3H-6,9-(epiminomethano)oxazolo[3,4-a]azepine-11-carboxylate (275 mg, 0.972 mmol) and NaOH (585 mg, 14.6 mmol) in ethanol (9 mL) and water (3 mL) was stirred at 80° C. for 12 hour. The solvent was concentrated under vacuum. The residue was purified by reverse phase flash chromatography on pre-packed C18 column (gradient: 0-100% CH₃CN in water (0.05% NH₄HCO₃)) to yield 250 mg of the title compound as a white solid. LC-MS: (ESI, m/z): [M+H]⁺=257. ¹H NMR (300 MHz, DMSO-d₆) δ 4.60 (br, 1H), 3.91-4.08 (m, 1H), 3.40-3.01 (m, 4H), 3.00-2.90 (m, 1H), 2.60-2.45 (m, 2H), 2.00 (br, 1H), 1.95-1.85 (m, 1H), 1.80-1.55 (m, 3H), 1.35 (s, 9H), 1.30-1.20 (m, 1H).

### Intermediate 39: (S,Z)-(2-(Fluoromethylene)tetrahydro-1H-pyrrolizin-7a(5H)-yl)methanol

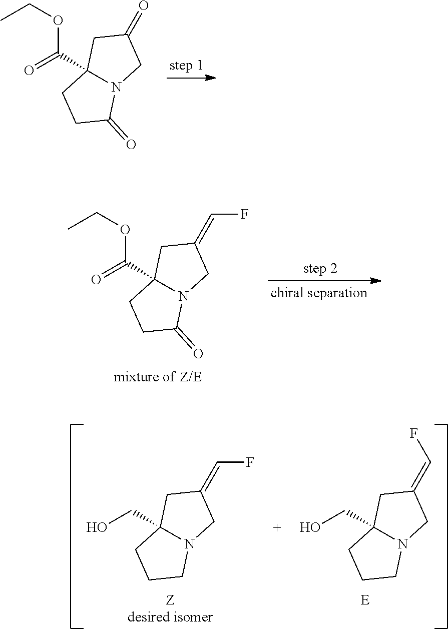

### Step 1: Ethyl (S,Z/E)-2-(fluoromethylene)-5-oxotetrahydro-1H-pyrrolizine-7a(5H)-carboxylate

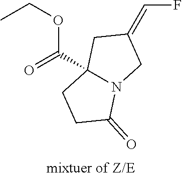 [SMILES: CCOC(=O)[C@@]12CCC(=O)N1C/C(=C\F)C2]

Under N₂, to a solution of 2-((fluoromethyl)sulfonyl)pyridine (177 mg, 1.01 mmol) in tetrahydrofuran (10 mL) was added KHMDS (1.2 mL, 1.20 mmol) at −78° C. The reaction system was stirred 30 min at −78° C., ethyl (S)-2,5-dioxotetrahydro-1H-pyrrolizine-7a(5H)-carboxylate (200 mg, 0.950 mmol, Intermediate 23, step 1, the faster peak) in THF (5 mL) was added slowly at −78° C. After stirring for 3 hours at −78° C., the reaction system was warm to room temperate and stirred another 1 hour at room temperature. The reaction was quenched with aqueous saturated ammonium chloride (1 mL), followed by 3 M HCl (2 mL). The solution was stirred for 1 hour at room temperature. The reaction was diluted with water, extracted with EtOAc. The combined organic layers were dried over anhydrous sodium sulfate and concentrated under vacuum. The residue was purified by flash chromatography on silica gel (gradient: 0%-30% ethyl acetate/petroleum ether) to yield 65.0 mg (30% yield) of the title compound as a yellow oil. LC-MS: (ES, m/z): [M+1]⁺=228.1. ¹H NMR (400 MHz, Chloroform-d₁, ppm) δ 6.75-6.65 (m, 1H), 6.55-6.44 (m, 1H), 4.45-4.32 (m, 2H), 4.27-4.20 (m, 4H), 3.90 (d, J=16 Hz, 1H), 3.73 (d, J=16 Hz, 1H), 3.32 (d, J=16 Hz, 1H), 3.04 (d, J=16 Hz, 1H), 2.89-2.75 (m, 2H), 2.72-2.56 (m, 2H), 2.53-2.35 (m, 4H), 2.25-2.08 (m, 2H), 1.33-1.28 (m, 6H). (diploid H due to it is a mixture of Z/E)

### Step 2: (S,Z)-(2-(Fluoromethylene)tetrahydro-1H-pyrrolizin-7a(5H)-yl)methanol and (S,E)-(2-(fluoromethylene)tetrahydro-1H-pyrrolizin-7a(5H)-yl)methanol

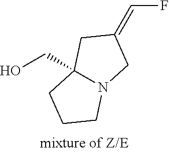 [SMILES: OC[C@@]12CCCN1C/C(=C\F)C2]

Under nitrogen, to a solution of ethyl (S,Z/E)-2-(fluoromethylene)-5-oxotetrahydro-1H-pyrrolizine-7a(5H)-carboxylate (60.0 mg, 0.260 mmol) in tetrahydrofuran (5 mL) was added DIBAL-H (2.64 mL, 2.64 mmol) at 0° C. The resulting solution was stirred for 1 hour at room temperature. The reaction was quenched with Na₂SO₄·10H₂O. After filtration, the filtrate was concentrated under reduced pressure. The residue was purified by flash chromatography on silica gel (gradient: 0%-20% MeOH/DCM (0.1% Et₃N)) to yield 26.0 mg (mixture of Z/E, two peaks on Flash, but collected together) (57% yield) of the title compound as a yellow oil. LC-MS: (ESI, m/z): [M+H]⁺=172.1. ¹H NMR (300 MHz, Chloroform-d₁, ppm) δ 6.79-6.38 (m, 2H), 3.92-3.70 (m, 2H), 3.61-3.20 (m, 8H), 2.79-2.44 (m, 5H), 2.36-2.26 (m, 1H), 2.14-1.66 (m, 8H). (diploid H due to it is a mixture of Z/E).

For scale up batch, 2.20 g (Z, the desired isomer) and 2.70 g (E, the undesired isomer) were obtained from 9.7 g ethyl (S,Z/E)-2-(fluoromethylene)-5-oxotetrahydro-1H-pyrrolizine-7a(5H)-carboxylate following the same procedure and purification method described above. Both are yellow oil.

Z isomer: LC-MS: (ESI, m/z): [M+H]⁺=172. ¹H NMR (300 MHz, Chloroform-d₁, ppm) δ 6.64-6.29 (m, 1H), 3.79-3.65 (m, 1H), 3.48-3.37 (m, 1H), 3.35-3.23 (m, 2H), 3.14-3.03 (m, 1H), 2.69-2.58 (m, 1H), 2.46-2.35 (m, 1H), 2.30-2.17 (m, 1H), 2.03-1.59 (m, 4H).

E isomer LC-MS: (ESI, m/z): [M+H]⁺=172. ¹H NMR (300 MHz, Chloroform-d₁, ppm) δ 6.76-6.42 (m, 1H), 3.75-3.65 (m, 1H), 3.48-3.34 (m, 2H), 3.33-3.18 (m, 2H), 2.73-2.40 (m, 3H), 2.09-1.65 (m, 4H).

### Example 1: Compound 1A and Compound 1B: 6-((5aS,6S,9R)-3-Chloro-1-fluoro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-4-methyl-5-(trifluoromethyl)pyridin-2-amine (two single unknown atropisomers)

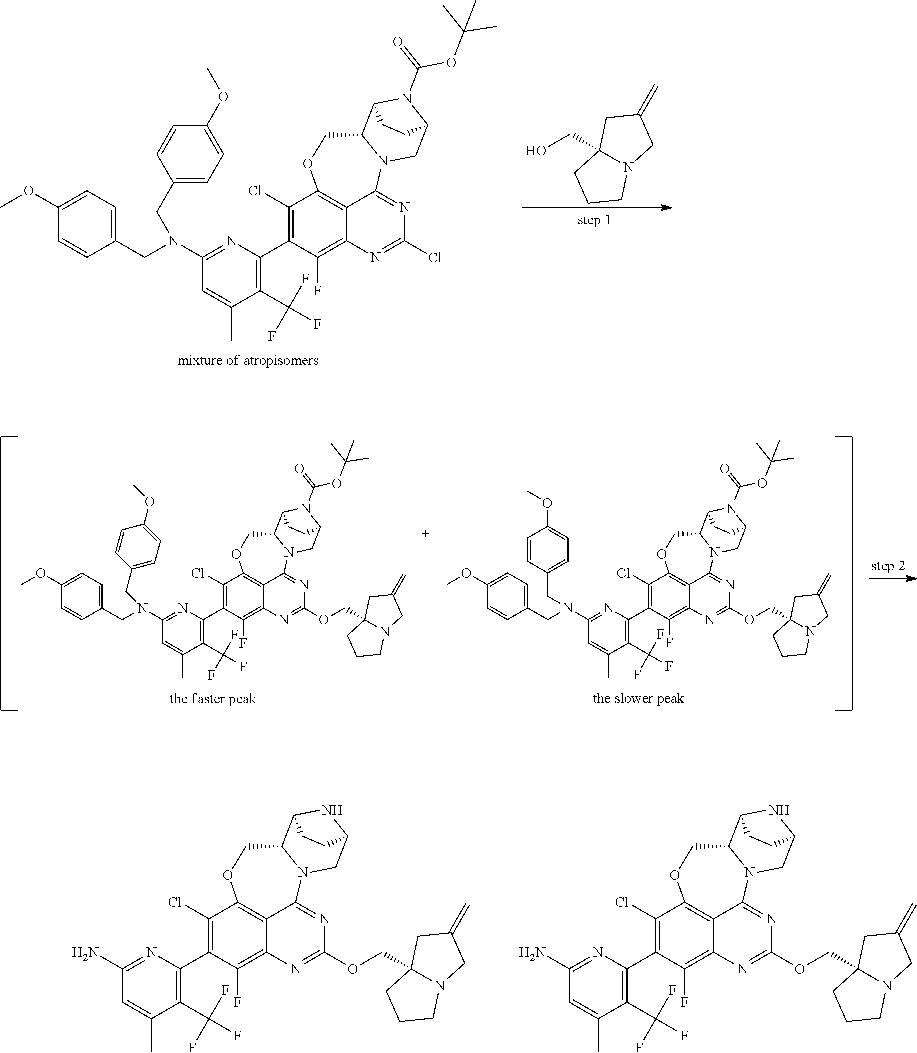

### Step 1: tert-Butyl (5aS,6S,9R)-2-(6-(bis(4-methoxybenzyl)amino)-4-methyl-3-(trifluoromethyl)pyridin-2-yl)-3-chloro-1-fluoro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate (mixture of atropisomers)

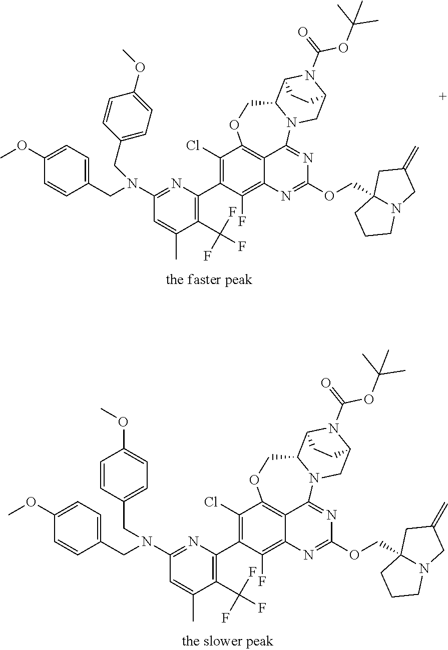

To an ice-cooled solution of (S)-(2-methyl enetetrahydro-1H-pyrrolizin-7a(5H)-yl)methanol (31.0 mg, 0.202 mmol, Intermediate 15) in tetrahydrofuran (4 mL) was added NaH (52.0 mg, 1.30 mmol). The resulting solution was warmed to room temperature. After 0.5 h, tert-butyl (5aS,6S,9R)-2-(6-(bis(4-methoxybenzyl)amino)-4-methyl-3-(trifluoromethyl) pyridin-2-yl)-3,13-dichloro-1-fluoro-5a,6,7,8,9,10-hexahydro-5H-6,9-epiminoazepino[2′,1′:3,4][1,4]oxazepino[5,6,7-de]quinazoline-15-carboxylate (117 mg, 0.134 mmol, racemic mixture of Intermediate 5) was added, and the resulting mixture was heated at 40° C. After 2 h, the reaction was quenched with saturated aqueous NH₄Cl solution and extracted with EtOAc. The combined organics were dried over anhydrous sodium sulfate and concentrated under vacuum. The residue was purified by pre-packed C18 column (solvent gradient: 0-100% ACN in water (0.05% TFA)) to yield a white solid (110 mg, 83% yield, mixture of atropisomers). The mixture was separated by Chiral-HPLC (Column: CHIRALPAK IC, 2×25 cm, 5 μm; Mobile Phase A: Hex (0.5% 2M NH₃-MeOH), Mobile Phase B: EtOH; Flow rate: 20 mL/min; Gradient: 20% B to 20% B in 13 min; 220/254 nm; RT₁:9.427; RT₂:10.666) to yield 30.0 mg of the faster peak and 35.0 mg of the slower peak as white solids. LC-MS: (ESI, m/z): [M+H]⁺=986.5.

### Step 2: 6-((5aS,6S,9R)-3-Chloro-1-fluoro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-4-methyl-5-(trifluoromethyl)pyridin-2-amine (two single unknown atropisomers)

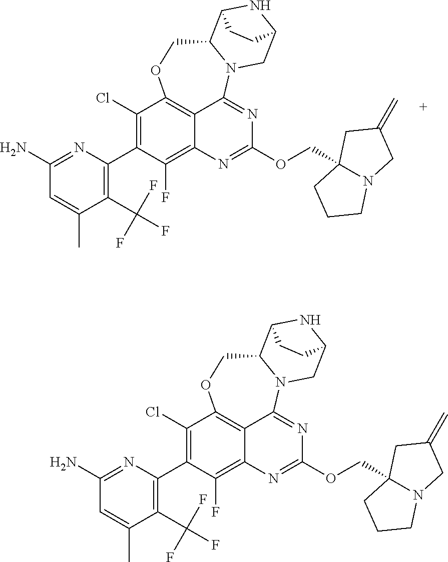 [SMILES: C=C1CN2CCC[C@@]2(COc2nc3c4c(c(Cl)c(-c5nc(N)cc(C)c5C(F)(F)F)c(F)c4n2)OC[C@@H]2[C@@H]4CC[C@H](CN32)N4)C1 ; C=C1CN2CCC[C@@]2(COc2nc3c4c(c(Cl)c(-c5nc(N)cc(C)c5C(F)(F)F)c(F)c4n2)OC[C@@H]2[C@@H]4CC[C@H](CN32)N4)C1]

A solution of tert-butyl (5aS,6S,9R)-2-(6-(bis(4-methoxybenzyl)amino)-4-methyl-3-(trifluoromethyl)pyridin-2-yl)-3-chloro-1-fluoro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate (the faster peak of step 1, 0.030 g, 0.030 mmol)) in 2,2,2-trifluoroacetic acid (3 mL) was stirred at 50° C. for 3 h. The reaction system was diluted with toluene (2 mL) and concentrated under vacuum. The residue was purified by Prep-HPLC (Column: XBridge Prep OBD C18 Column, 30×150 mm 5 μm; Mobile Phase A: Water (10 mmol/L NH₄HCO₃), Mobile Phase B: ACN; Flow rate: 60 mL/min; Gradient: 38% B to 68% B in 7 min; 254 nm; RT: 6.12) to yield 1A as a white solid (13.5 mg, 68% yield) of. LC-MS: (ESI, m/z): [M+H]⁺=646.3. ¹H NMR (400 MHz, DMSO-d₆, ppm) δ 6.81 (s, 2H), 6.47 (s, 1H), 4.90 (s, 2H), 4.78-4.71 (m, 1H), 4.59-4.51 (m, 1H), 4.39-4.32 (m, 1H), 4.03 (d, J=10.8 Hz, 1H), 4.00-3.95 (m, 1H), 3.91 (d, J=10.4 Hz, 1H), 3.59-3.50 (m, 2H), 3.49-3.42 (m, 1H), 3.23-3.12 (m, 1H), 3.10-2.94 (m, 2H), 2.62-2.56 (m, 2H), 2.55-2.53 (m, 1H), 2.36 (s, 3H), 2.00-1.91 (m, 1H), 1.91-1.82 (m, 1H), 1.82-1.74 (m, 2H), 1.71-1.61 (m, 3H), 1.60-1.50 (m, 1H).

A solution of tert-butyl (5aS,6S,9R)-2-(6-(bis(4-methoxybenzyl)amino)-4-methyl-3-(trifluoromethyl)pyridin-2-yl)-3-chloro-1-fluoro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate (the slower peak of step 1, 0.039 g, 0.039 mmol) in 2,2,2-trifluoroacetic acid (3 mL) was stirred at 50° C. for 3 h. The reaction mixture was diluted with toluene (2 mL) and concentrated under vacuum. The residue was purified by Prep-HPLC (Column: XBridge Prep OBD C18 Column, 30×150 mm 5 μm; Mobile Phase A: Water (10 mmol/L NH₄HCO₃), Mobile Phase B: ACN; Flow rate: 60 mL/min; Gradient: 36% B to 66% B in 7 min; 254 nm; R_{T}: 6.07) to yield 1B as a white solid (14.9 mg, 58% yield) of. LC-MS: (ESI, m/z): [M+H]⁺=646.3. ¹H NMR (300 MHz, DMSO-d₆, ppm) δ 6.81 (s, 2H), 6.47 (s, 1H), 4.93-4.80 (m, 3H), 4.63-4.53 (m, 1H), 4.35-4.22 (m, 1H), 4.09-3.88 (m, 3H), 3.62-3.50 (m, 2H), 3.47-3.41 (m, 1H), 3.22-3.12 (m, 1H), 3.09-2.93 (m, 2H), 2.63-2.54 (m, 2H), 2.41-2.30 (m, 4H), 2.02-1.90 (m, 1H), 1.89-1.74 (m, 2H), 1.73-1.61 (m, 4H), 1.60-1.50 (m, 1H).

### Example 2: Compound 2: 6-((2R,5aS,6S,9R)-3-Chloro-1-fluoro-13-(((S,Z)-2-(fluoromethylene)tetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-4-methyl-5-(trifluoromethyl)pyridin-2-amine (single known atropisomer)

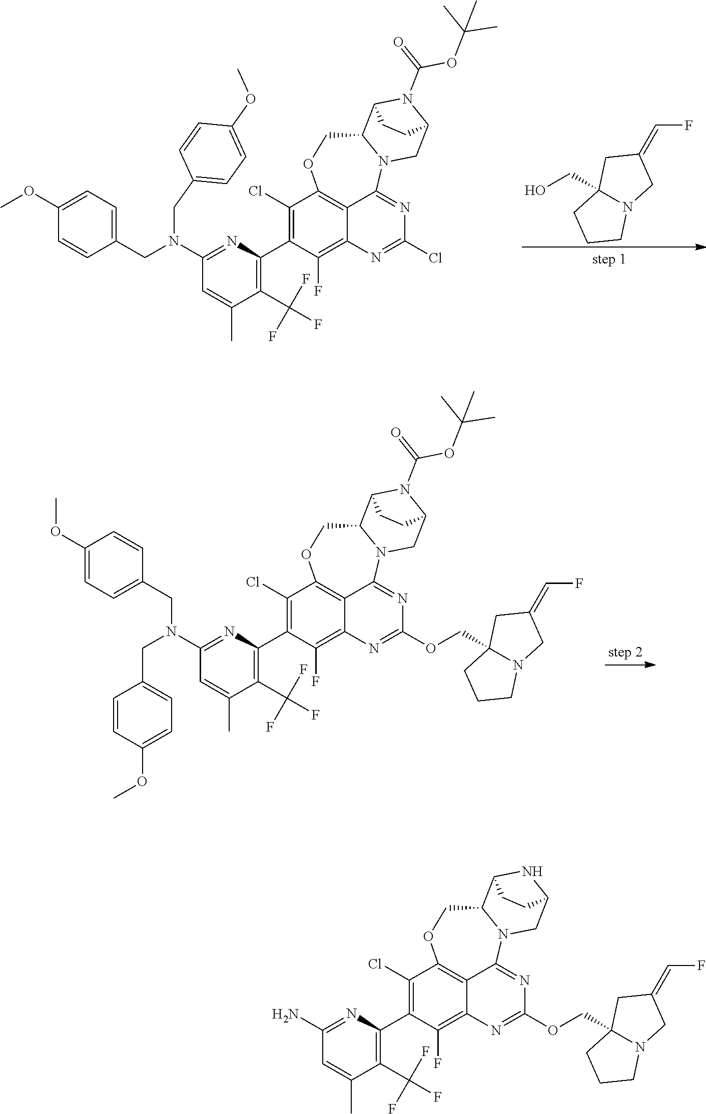

### Step 1: tert-Butyl (2R,5aS,6S,9R)-2-(6-(bis(4-methoxybenzyl)amino)-4-methyl-3-(trifluoromethyl)pyridin-2-yl)-3-chloro-1-fluoro-13-(((S,Z)-2-(fluoromethylene)tetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate (single known atropisomer)

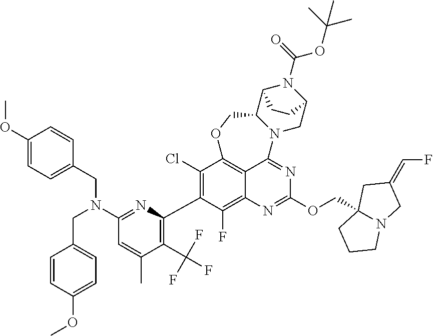

To a ice-cooled solution of (S,Z)-(2-(fluoromethylene)tetrahydro-1H-pyrrolizin-7a(5H)-yl)methanol (502 mg, 2.93 mmol, Intermediate 21) in tetrahydrofuran (20 mL) under nitrogen was added NaH (470 mg, 11.7 mmol). The resulting solution was warmed to room temperature. After 0.5 h, tert-butyl (2R,5aS,6S,9R)-2-(6-(bis(4-methoxybenzyl)amino)-4-methyl-3-(trifluoromethyl)pyridin-2-yl)-3,13-dichloro-1-fluoro-5a,6,7,8,9,10-hexahydro-5H-6,9-epiminoazepino[2′,1′:3,4][1,4]oxazepino[5,6,7-de]quinazoline-15-carboxylate (1.70 g, 1.95 mmol, Intermediate 5) was added, and the resulting mixture was warmed to 40° C. for 3 h. The reaction mixture was added saturated aqueous NH₄Cl solution (100 mL) and extracted with EtOAc (3×50 mL). The combined organic were dried over anhydrous sodium sulfate and concentrated under vacuum. The residue was purified by pre-packed C18 column (solvent gradient: 0-100% ACN in water (0.05% TFA)) to yield the title compound as a yellow solid (1.74 g, 88% yield). LC-MS: (ESI, m/z): [M+H]⁺=1004.5.

### Step 2: 6-((2R,5aS,6S,9R)-3-Chloro-1-fluoro-13-(((S,Z)-2-(fluoromethylene)tetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-4-methyl-5-(trifluoromethyl)pyridin-2-amine

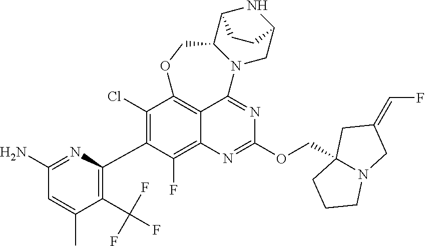

A solution of tert-butyl (2R,5aS,6S,9R)-2-(6-(bis(4-methoxybenzyl)amino)-4-methyl-3-(trifluoromethyl)pyridin-2-yl)-3-chloro-1-fluoro-13-(((S,Z)-2-(fluoromethylene)tetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate (1.74 g, 1.73 mmol) in 2,2,2-trifluoroacetic acid (20 mL) was stirred at 50° C. for 3.5 h. The reaction mixture was diluted with toluene (10 mL) and concentrated under vacuum. The residue was dissolved with DMF (6 mL) and added dropwise to 0.24 M NaHCO₃ solution (500 mL). The solids were collected, and the crude product was purified by reverse phase flash chromatography on C18 column (solvent gradient: 0-100% MeCOH in water (5 mmol/L NH₄HCO₃)) to yield the title compound as a white solid (863 mg, 77% yield). LC-MS: (ESI, m/z): [M+H]⁺=664.25. ¹H NMR (400 MHz, DMSO-d₆, ppm) δ 6.99-6.61 (m, 3H), 6.47 (s, 1H), 4.85-4.71 (m, 1H), 4.60-4.51 (m, 1H), 4.46-4.31 (m, 1H), 4.07 (d, J=10.4 Hz, 1H), 4.03-3.91 (m, 2H), 3.74-3.69 (m, 1H), 3.65-3.59 (m, 1H), 3.57-3.48 (m, 1H), 3.30-3.26 (m, 1H), 3.12-2.95 (m, 2H), 2.61-2.52 (m, 2H), 2.41-2.31 (m, 4H), 1.99-1.91 (m, 1H), 1.91-1.74 (m, 3H), 1.73-1.61 (m, 3H), 1.60-1.50 (m, 1H).

### Example 3: Compound 3: 6-((2R,5aS,6S,9R)-3-Chloro-1-fluoro-15-methyl-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-4-methyl-5-(trifluoromethyl)pyridin-2-amine

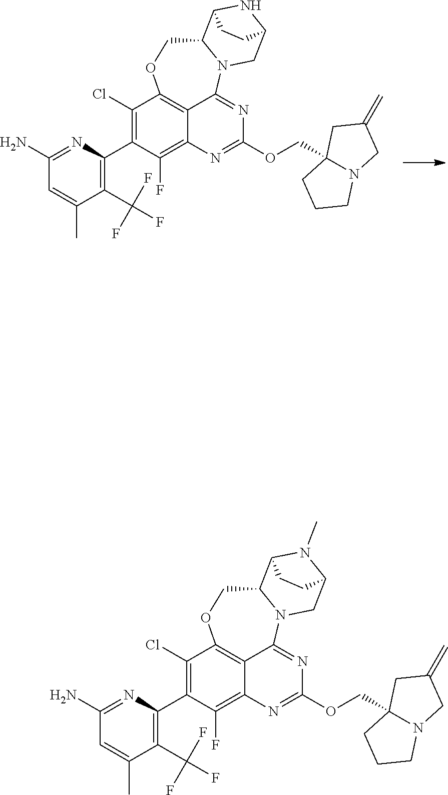

To a solution of 6-((2R,5aS,6S,9R)-3-chloro-1-fluoro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-4-methyl-5-(trifluoromethyl)pyridin-2-amine (0.040 g, 0.060 mmol, (Compound 1A)) in methanol (3 mL) was added HCHO (37% in water) (6.6 mg, 0.080 mmol) and NaOAc (5.0 mg, 0.060 mmol) at room temperature. After 1 h, NaBH₃CN (11.7 mg, 0.190 mmol) was added. The reaction mixture was maintained at room temperature for 1 hour and concentrated under reduced pressure. The residue was purified by Prep-HPLC (Column: XBridge Prep OBD C18 Column, 30×150 mm 5 μm; Mobile Phase A: Water (10 mmol/L NH₄HCO₃), Mobile Phase B: ACN; Flow rate: 60 mL/min; Gradient: 35% B to 65% B in 7 min; 254 nm; R_{T}: 6.5) to yield the title compound as a white solid (9.4 mg, 23% yield). LC-MS: (ESI, m/z): [M+H]⁺=660. ¹H NMR (400 MHz, DMSO-d₆, ppm) δ 6.83 (s, 2H), 6.47 (s, 1H), 4.92 (s, 2H), 4.80-4.70 (m, 1H), 4.60-4.49 (m, 1H), 4.41-4.36 (m, 1H), 4.15-3.90 (m, 3H), 3.70-3.55 (m, 1H), 3.30-3.20 (m, 3H), 3.19-3.10 (m, 1H), 3.03 (s, 1H), 2.76-2.58 (m, 2H), 2.45-2.31 (m, 4H), 2.26 (s, 3H), 2.08-1.48 (m, 8H).

### Example 4: Compound 4: 1-((2R,5aS,6S,9R)-2-(6-Amino-4-methyl-3-(trifluoromethyl)pyridin-2-yl)-3-chloro-1-fluoro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-15-yl)ethan-1-one

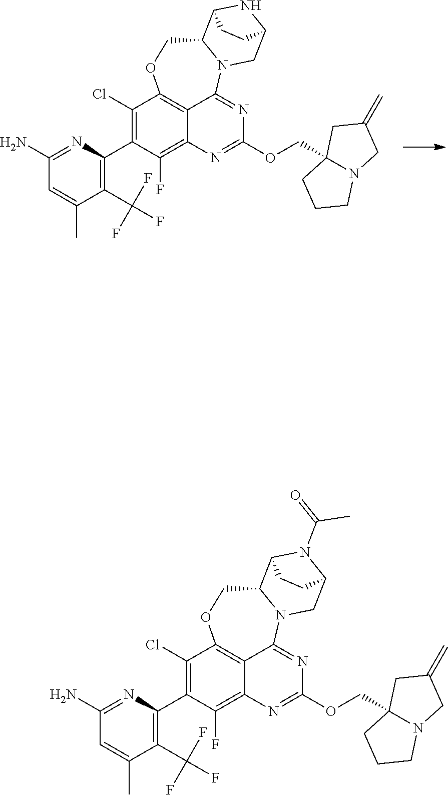

To a solution of 6-((2R,5aS,6S,9R)-3-chloro-1-fluoro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-4-methyl-5-(trifluoromethyl)pyridin-2-amine (0.040 g, 0.060 mmol, (Compound 1A)) and DIPEA (0.040 g, 0.310 mmol) in dichloromethane (3 mL) was added Ac₂O (7.6 mg, 0.070 mmol) at room temperature. After 1 h, the reaction mixture was concentrated under reduced pressure. The residue was purified by Prep-HPLC (Column: Atlantis HILIC OBD Column, 19×150 mm×5 um; Mobile Phase A: Water (10 mmol/L NH₄HCO₃), Mobile Phase B: ACN; Flow rate: 60 mL/min; Gradient: 32% B to 62% B in 9 min, 254/220 nm; Rr: 8.27) to yield 10.0 mg (23% yield) of the title compound as a white solid. LC-MS: (ESI, m/z): [M+H]⁺=688. ¹H NMR (400 MHz, DMSO-d₆, ppm) δ 6.83 (s, 2H), 6.48 (s, 1H), 5.09-4.77 (m, 3H), 4.74-4.39 (m, 4H), 4.24-3.89 (m, 3H), 3.70-3.55 (m, 1H), 3.29-3.20 (m, 1H), 3.15-2.96 (m, 2H), 2.75-2.58 (m, 2H), 2.48-2.28 (m, 4H), 2.09 (s, 3H), 2.04-1.61 (m, 8H).

Each compound in Table below was prepared following a similar experimental procedure (using appropriately substituted reagents) as described Example 2.

| Cmpd | |
|---|---|
| No | Structure |
| 1a | 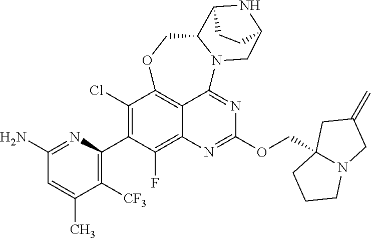 |
| 1b | 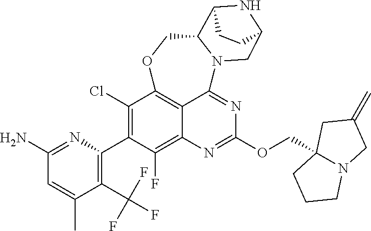 |
| 2 | 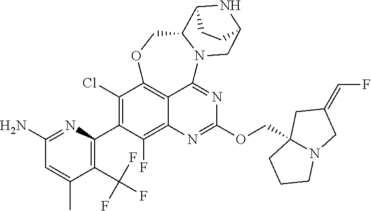 |
| 5 | 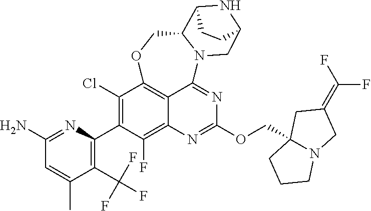 |
| 6A | 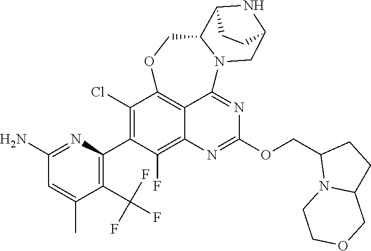 |
| 6B | 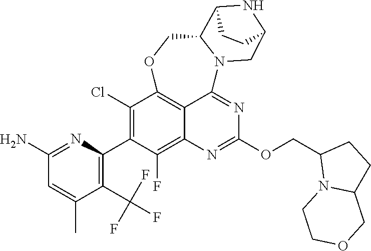 |
| 7 | 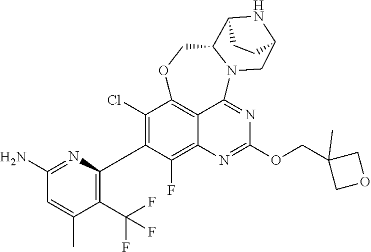 |
| 8 | 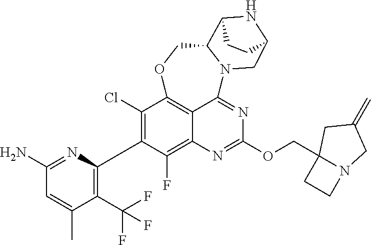 |
| 9 | 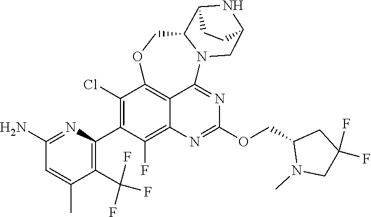 |
| 3 | 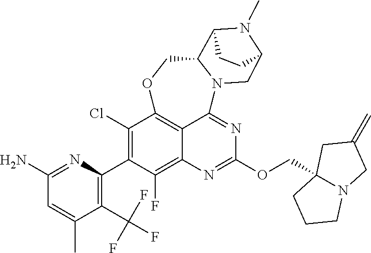 |
| 4 | 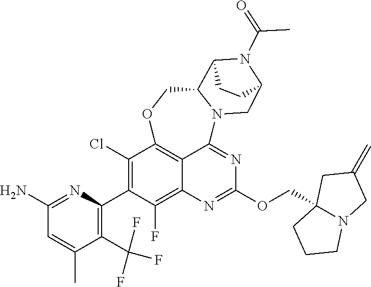 |
| 10 | 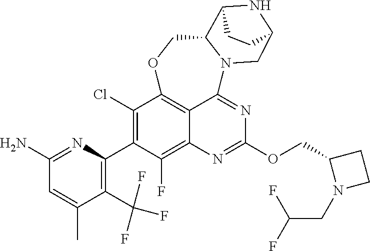 |
| 11 | 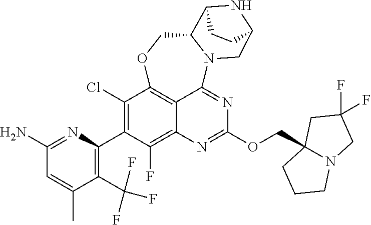 |
| 12 | 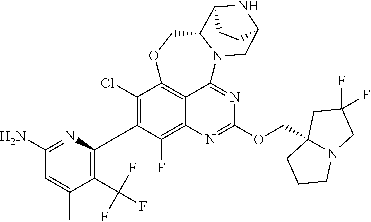 |

### Example 13: Compound 13A & Compound 13B: 6-((13aR)-11-Chloro-9-fluoro-7-(((S)-2-methylenetetrahydro-1H-pyrrolizin-7a(5H)-yl)methoxy)-1,2,3,4,13,13a-hexahydropyrazino[2′,1′:3,4][1,4]oxazepino[5,6,7-de]quinazolin-10-yl)-4-methyl-5-(trifluoromethyl)pyridin-2-amine (two single unknown atropisomers)

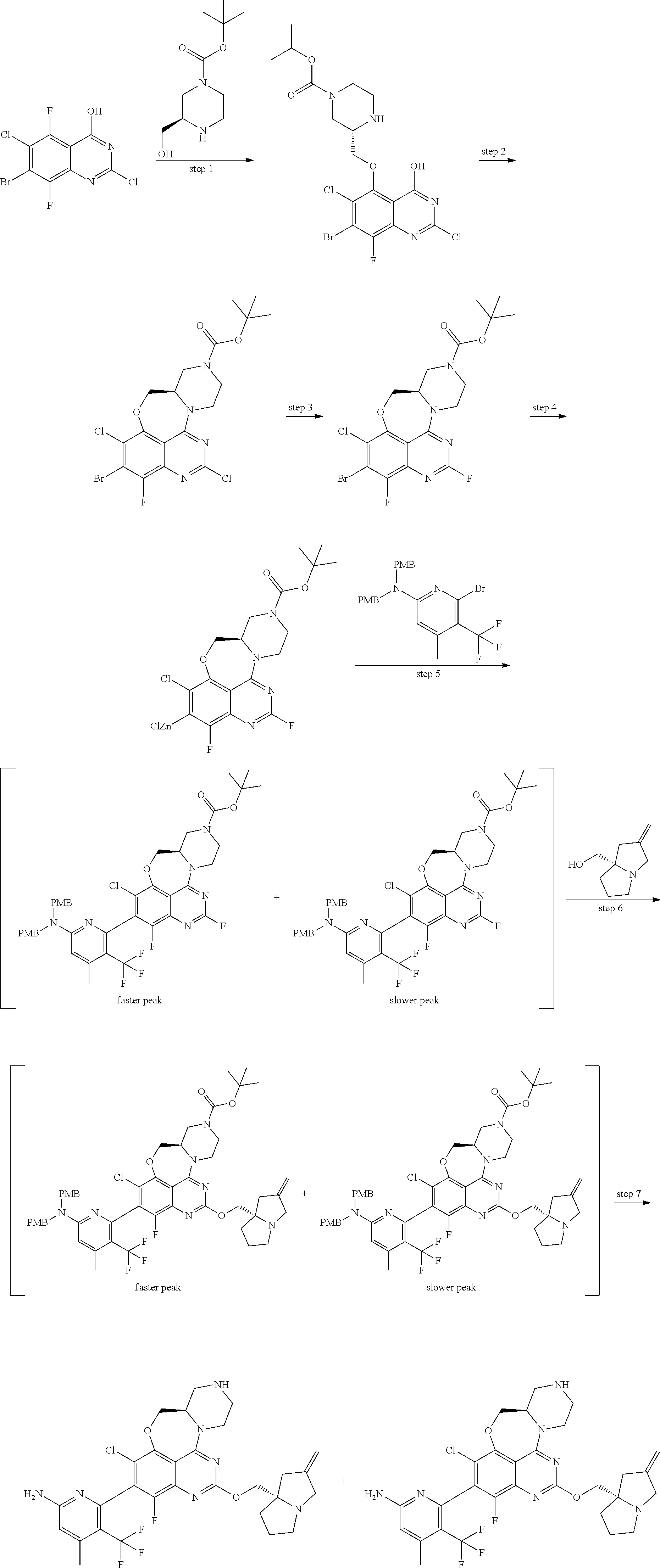

### Step 1: tert-Butyl (S)-3-(((7-bromo-2,6-dichloro-8-fluoro-4-hydroxyquinazolin-5-yl)oxy)methyl)piperazine-1-carboxylate

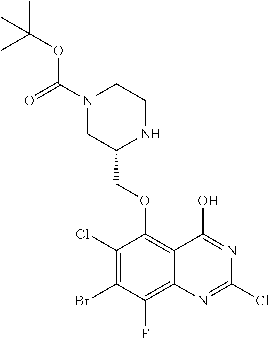 [SMILES: CC(C)(C)OC(=O)N1CCN[C@H](COc2c(Cl)c(Br)c(F)c3nc(Cl)nc(O)c23)C1]

Under nitrogen, to an ice-cooled solution of tert-butyl (R)-3-(hydroxymethyl)piperazine-1-carboxylate (444 mg, 2.05 mmol) in THF (10 mL) was added NaH (274 mg, 6.85 mmol, 60% in mineral oil). The resulting solution was warmed to 25° C. After 30 min, 7-bromo-2,6-dichloro-5,8-difluoroquinazolin-4-ol (750 mg, 2.28 mmol, Intermediate 2) was added, and the reaction mixture was warmed to 65° C. for 1 h. The reaction was quenched with saturated aqueous NH₄Cl solution and extracted with ethyl acetate (3×). The combined organic layers were dried over anhydrous sodium sulfate and concentrated under vacuum. The residue was purified by pre-packed C18 column (solvent gradient: 0-100% ACN in water (0.05% TFA)) to yield a white solid (320 mg, 26% yield). LC-MS: (ESI, m/z): [M+H]⁺=525.1

### Step 2: tert-Butyl (R)-10-bromo-7,11-dichloro-9-fluoro-3,4,13,13a-tetrahydropyrazino[2′,1′:3,4][1,4]oxazepino[5,6,7-de]quinazoline-2 (1H)-carboxylate

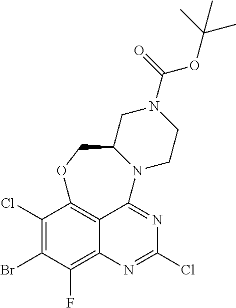 [SMILES: CC(C)(C)OC(=O)N1CCN2c3nc(Cl)nc4c(F)c(Br)c(Cl)c(c34)OC[C@H]2C1]

A solution of tert-butyl (S)-3-(((7-bromo-2,6-dichloro-8-fluoro-4-hydroxyquinazolin-5-yl)oxy)methyl)piperazine-1-carboxylate (320 mg, 0.610 mmol), DIPEA (1.18 g, 9.14 mmol) and BOP-Cl (623 mg, 2.44 mmol) in dichloromethane (20 mL) was stirred at 25° C. for 3 h. The reaction was partitioned between DCM and brine. The collected organic layer was dried over anhydrous sodium sulfate and concentrated under vacuum. Purification by flash column chromatography on silica gel (gradient: 0-30% ethyl acetate/petroleum ether) afforded the title compound as a white solid (190 mg, 61% yield). LC-MS: (ESI, m/z): [M+H]⁺=507.0.

### Step 3: tert-Butyl (R)-10-bromo-11-chloro-7,9-difluoro-3,4,13,13a-tetrahydropyrazino[2′,1′:3,4][1,4]oxazepino[5,6,7-de]quinazoline-2 (1H)-carboxylate

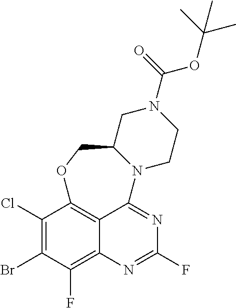 [SMILES: CC(C)(C)OC(=O)N1CCN2c3nc(F)nc4c(F)c(Br)c(Cl)c(c34)OC[C@H]2C1]

Under nitrogen, a mixture of tert-butyl (R)-10-bromo-7,11-dichloro-9-fluoro-3,4,13,13a-tetrahydropyrazino[2′,1′:3,4][1,4]oxazepino[5,6,7-de]quinazoline-2 (1H)-carboxylate (270 mg, 0.533 mmol) and CsF (162 mg, 1.06 mmol) in DMSO (5 mL) was stirred at 50° C. for 3 h. The mixture was cooled to ambient temperature and diluted with EtOAc (50 mL). The resulting solution was washed with saturated aqueous NaCl solution. The collected organic layer was dried over anhydrous sodium sulfate and concentrated under vacuum. Purification by flash chromatography on silica gel (gradient: 0-30% ethyl acetate/petroleum ether) provided a yellow solid (190 mg, 72% yield). LC-MS: (ESI, m/z): [M+H]⁺=491.1.

### Step 4 (R)-(2-(tert-Butoxycarbonyl)-11-chloro-7,9-difluoro-1,2,3,4,13,13a-hexahydropyrazino[2′,1′:3,4][1,4]oxazepino[5,6,7-de]quinazolin-10-yl)zinc(II) chloride

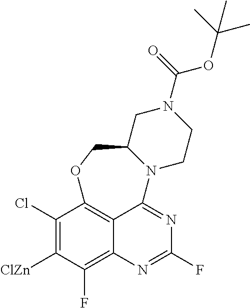

To a solution of tert-butyl (R)-10-bromo-11-chloro-7,9-difluoro-3,4,13,13a-tetrahydropyrazino[2′,1′:3,4][1,4]oxazepino[5,6,7-de]quinazoline-2 (1H)-carboxylate (140 mg, 0.280 mmol) in anhydrous THF (0.6 mL) was added iPrMgCl·LiCl (0.22 mL, 0.286 mmol, 1.3 M in THF) dropwise at −78° C. The solution was maintained at −78° C. for 1 h. ZnCl₂ (0.15 mL, 0.30 mmol, 2 M in 2-methyltetrahydrofuran) was added into the solution at −78° C. dropwise. After 10 min, the solution was warmed to room temperature. After 1 h, the reaction mixture was used in the following step.

### Step 5: tert-Butyl (13aR)-10-(6-(bis(4-methoxybenzyl)amino)-4-methyl-3-(trifluoromethyl)pyridin-2-yl)-11-chloro-7,9-difluoro-3,4,13,13a-tetrahydropyrazino[2′,1′:3,4][1,4]oxazepino[5,6,7-de]quinazoline-2 (1H)-carboxylate (two single unknown atropisomers)

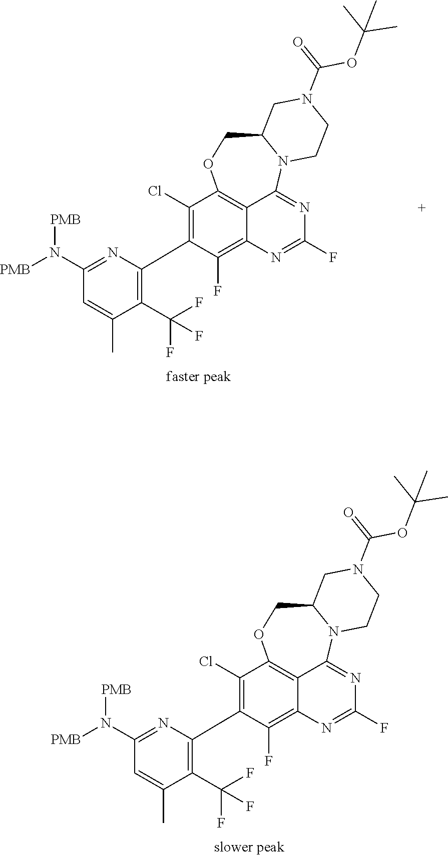

The zincate solution from previous step was transferred into a degassed solution of 6-bromo-N,N-bis(4-methoxybenzyl)-4-methyl-5-(trifluoromethyl)pyridin-2-amine (117 mg, 0.240 mmol), and PdCl₂(PPh₃)₂ (6.70 mg, 0.010 mmol) in anhydrous THF (0.6 mL). The suspension was heated at 50° C. After 18 h, the reaction was added to water (30 mL). The resulting solution was extracted with EtOAc (3×). The combined organic layers were dried over anhydrous sodium sulfate and concentrated under vacuum. Purification by flash chromatography on silica gel (gradient: 0-30% ethyl acetate/petroleum ether) afforded a white solid (130 mg, mixture of two atropisomers). The mixture was separated by Chiral-HPLC (Column: CHIRALPAK IC, 2×25 cm, 5 μm; Mobile Phase A: Hex (0.5% 2M NH₃-MeCOH), Mobile Phase B: EtOH; Flow rate: 20 mL/min; Gradient: 20% B to 20% B in 21 min; 220/254 nm; R_{T1}:13.429; R_{T2}:17.077) to yield 54.0 mg of the faster peak and 66.0 mg of the slower peak as white solids. LC-MS: (ESI, m/z): [M+H]⁺=827.4.

### Step 6: tert-Butyl (13aR)-10-(6-(bis(4-methoxybenzyl)amino)-4-methyl-3-(trifluoromethyl)pyridin-2-yl)-11-chloro-9-fluoro-7-(((S)-2-methylenetetrahydro-1H-pyrrolizin-7a(5H)-yl)methoxy)-3,4,13,13a-tetrahydropyrazino[2′,1′:3,4][1,4]oxazepino[5,6,7-de]quinazoline-2 (1H)-carboxylate (two single unknown atropisomers)

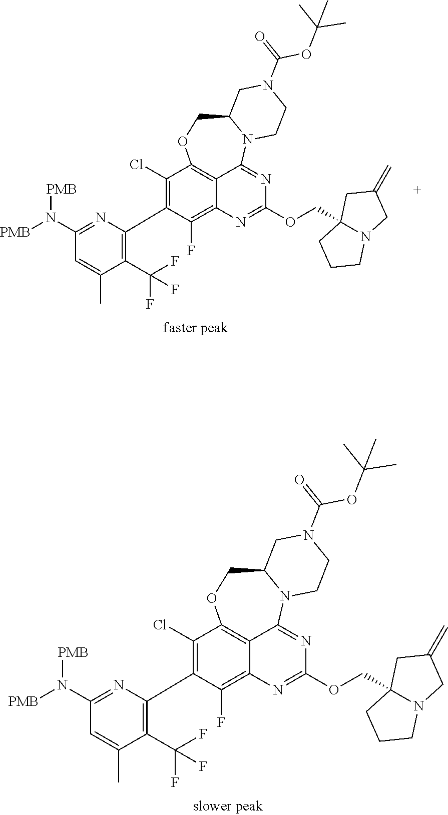

To an ice-cooled solution of (S)-(2-methylenetetrahydro-1H-pyrrolizin-7a(5H)-yl)methanol (15.0 mg, 0.098 mmol, Intermediate 15) in tetrahydrofuran (3 mL) was added NaH (15.7 mg, 0.392 mmol). The resulting solution was warmed to 25° C. After 30 min, tert-butyl (13aR)-10-(6-(bis(4-methoxybenzyl)amino)-4-methyl-3-(trifluoromethyl)pyridin-2-yl)-11-chloro-7,9-difluoro-3,4,13,13a-tetrahydropyrazino[2′,1′:3,4][1,4]oxazepino[5,6,7-de]quinazoline-2 (1H)-carboxylate (the faster peak of step 5, 54.0 mg, 0.065 mmol) was added to the reaction. After 2 h, the reaction was quenched with saturated aqueous NH₄Cl solution (30 mL) and extracted with EtOAc. The combined organic layers were dried over anhydrous sodium sulfate and concentrated under vacuum. Purification by flash chromatography on silica gel (gradient: 0-10% MeOH/DCM) provided a white solid (47.0 mg, 75% yield). LC-MS: (ESI, m/z): [M+H]⁺=960.5

Analogous to method described as above, the other atropisomer (slower peak) was prepared from tert-butyl (13aR)-10-(6-(bis(4-methoxybenzyl)amino)-4-methyl-3-(trifluoromethyl)pyridin-2-yl)-11-chloro-7,9-difluoro-3,4,13,13a-tetrahydropyrazino[2′,1′:3,4][1,4]oxazepino[5,6,7-de]quinazoline-2 (1H)-carboxylate (the slower peak of step 5) and (S)-(2-methylenetetrahydro-1H-pyrrolizin-7a(5H)-yl)methanol (Intermediate 15)

### Step 7: 6-((13aR)-11-Chloro-9-fluoro-7-(((S)-2-methylenetetrahydro-1H-pyrrolizin-7a(5H)-yl)methoxy)-1,2,3,4,13,13a-hexahydropyrazino[2′,1′:3,4][1,4]oxazepino[5,6,7-de]quinazolin-10-yl)-4-methyl-5-(trifluoromethyl)pyridin-2-amine (two single unknown atropisomers)

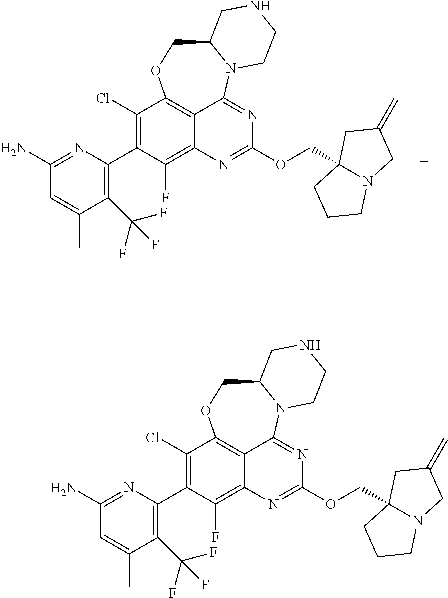 [SMILES: C=C1CN2CCC[C@@]2(COc2nc3c4c(c(Cl)c(-c5nc(N)cc(C)c5C(F)(F)F)c(F)c4n2)OC[C@H]2CNCCN32)C1 ; C=C1CN2CCC[C@@]2(COc2nc3c4c(c(Cl)c(-c5nc(N)cc(C)c5C(F)(F)F)c(F)c4n2)OC[C@H]2CNCCN32)C1]

A solution of tert-butyl (13aR)-10-(6-(bis(4-methoxybenzyl)amino)-4-methyl-3-(trifluoromethyl)pyridin-2-yl)-11-chloro-9-fluoro-7-(((S)-2-methylenetetrahydro-1H-pyrrolizin-7a(5H)-yl)methoxy)-3,4,13,13a-tetrahydropyrazino[2′,1′:3,4][1,4]oxazepino[5,6,7-de]quinazoline-2 (1H)-carboxylate (the faster peak of step 6, 47.0 mg, 0.049 mmol) in 2,2,2-trifluoroacetic acid (3 mL) was stirred at 50° C. for 3 h. The reaction mixture was diluted with toluene (2 mL) and concentrated under vacuum. Purification by Prep-HPLC (Column: XBridge Prep OBD C18 Column, 30×150 mm 5 μm; Mobile Phase A: Water (10 mmol/L NH₄HCO₃), Mobile Phase B: ACN; Flow rate: 60 mL/min; Gradient: 26% B to 56% B in 10 min; 254 nm; R_{T}: 9.02.) yielded a white solid (11.5 mg, 44% yield, Compound 13A). LC-MS: (ESI, m/z): [M+H]⁺=620.3. ¹H NMR (400 MHz, DMSO-d₆, ppm) δ 6.81 (s, 2H), 6.47 (s, 1H), 5.02-4.75 (m, 3H), 4.69-4.45 (m, 1H), 4.44-4.31 (m, 1H), 4.11-3.89 (m, 3H), 3.55 (d, J=14.0 Hz, 1H), 3.18 (d, J=13.6 Hz, 1H), 3.10-2.92 (m, 4H), 2.72-2.55 (m, 4H), 2.41-2.31 (m, 4H), 2.09-1.97 (m, 1H), 1.94-1.72 (m, 2H), 1.71-1.61 (m, 1H).

Analogous to method described above, the other atropisomer Compound 13B was prepared from tert-Butyl (13aR)-10-(6-(bis(4-methoxybenzyl)amino)-4-methyl-3-(trifluoromethyl)pyridin-2-yl)-11-chloro-9-fluoro-7-(((S)-2-methylenetetrahydro-1H-pyrrolizin-7a(5H)-yl)methoxy)-3,4,13,13a-tetrahydropyrazino[2′,1′:3,4][1,4]oxazepino[5,6,7-de]quinazoline-2 (1H)-carboxylate (the slower peak of step 6) LC-MS: (ESI, m/z): [M+H]⁺=620.3. ¹H NMR (400 MHz, DMSO-d₆, ppm) δ 6.82 (s, 2H), 6.48 (s, 1H), 4.89 (s, 2H), 4.82 (d, J=12.8 Hz, 1H), 4.46 (d, J=3.6 Hz, 2H), 3.97 (s, 2H), 3.83 (dd, J=10.4, 2.4 Hz, 1H), 3.55 (d, J=14.0 Hz, 1H), 3.19 (d, J=14.0 Hz, 1H), 3.09-2.94 (m, 4H), 2.78 (t, J=11.2 Hz, 1H), 2.71-2.62 (m, 1H), 2.61-2.53 (m, 2H), 2.41-2.30 (m, 4H), 2.09-1.94 (m, 1H), 1.93-1.73 (m, 2H), 1.72-1.61 (m, 1H)

### Example 14: Compound 14: 6-((5aS,6S,9R)-3-Chloro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-4-methyl-5-(trifluoromethyl)pyridin-2-amine

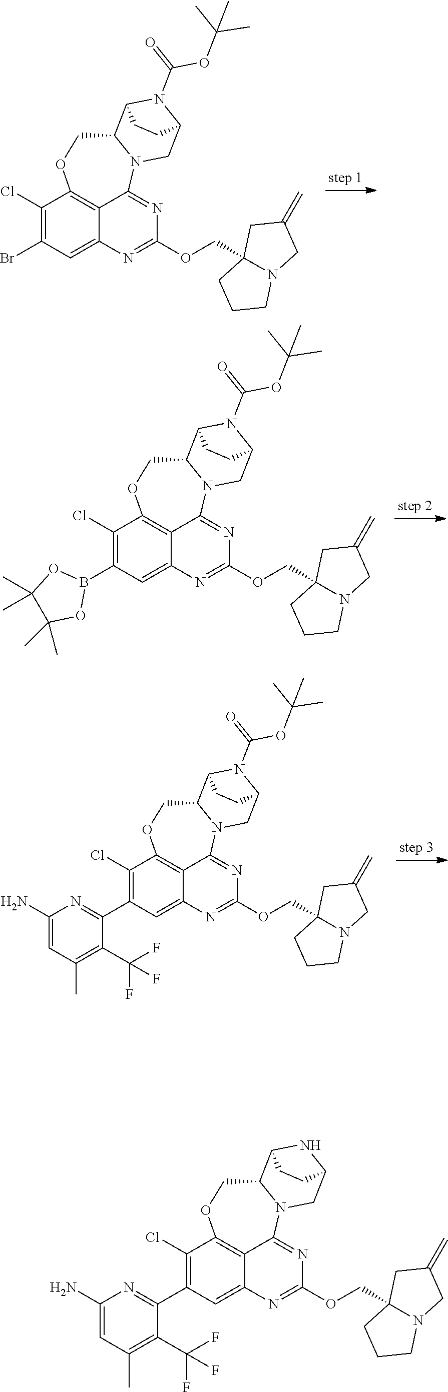

### Step 1 tert-Butyl (5aS,6S,9R)-3-chloro-13-(((S)-2-methylenetetrahydro-1H-pyrrolizin-7a(5H)-yl)methoxy)-2-(4,4,5,5-tetramethyl-1,3,2-dioxaborolan-2-yl)-5a,6,7,8,9,10-hexahydro-5H-6,9-epiminoazepino[2′,1′:3,4][1,4]oxazepino[5,6,7-de]quinazoline-15-carboxylate

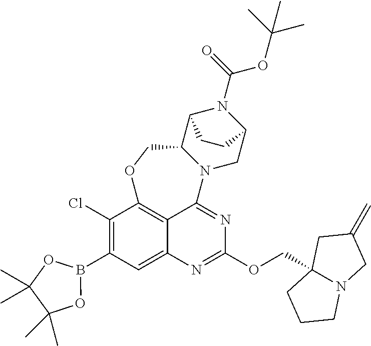

A suspension of tert-butyl (5aS,6S,9R)-2-bromo-3-chloro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate (208 mg, 0.330 mmol, Intermediate 9), B₂Pin₂ (261 mg, 1.03 mmol), Pd(dppf)Cl₂ (59.3 mg, 0.0800 mmol) and KOAc (113 mg, 1.16 mmol) in 1,4-dioxane (4 mL) was heated at 80° C. After 4 h, the reaction mixture was concentrated under vacuum. A mixture of petroleum ether (10 mL) and ethyl acetate (1 mL) was added to the residue, and the resulting suspension was stirred at room temperature for 0.5 h. The solids were filtered, and the filtrate was concentrated under reduced pressure to afford the crude title compound as a yellow solid (400 mg) of. LC-MS: (ESI, m/z) (only observed the Mass of the corresponding boronic acid): [M+H]⁺=598.

### Step 2: tert-Butyl (5aS,6S,9R)-2-(6-amino-4-methyl-3-(trifluoromethyl)pyridin-2-yl)-3-chloro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate

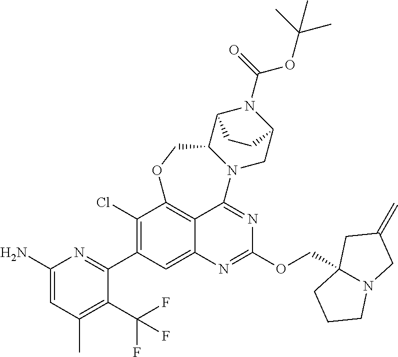 [SMILES: C=C1CN2CCC[C@@]2(COc2nc3c4c(c(Cl)c(-c5nc(N)cc(C)c5C(F)(F)F)cc4n2)OC[C@@H]2[C@@H]4CC[C@H](CN32)N4C(=O)OC(C)(C)C)C1]

A stirred solution of tert-butyl (5aS,6S,9R)-3-chloro-13-(((S)-2-methylenetetrahydro-1H-pyrrolizin-7a(5H)-yl)methoxy)-2-(4,4,5,5-tetramethyl-1,3,2-dioxaborolan-2-yl)-5a,6,7,8,9,10-hexahydro-5H-6,9-epiminoazepino[2′,1′:3,4][1,4]oxazepino[5,6,7-de]quinazoline-15-carboxylate (130 mg, 0.19 mmol), 6-bromo-4-methyl-5-(trifluoromethyl)pyridin-2-amine (24.7 mg, 0.100 mmol), Pd(PPh₃)₂Cl₂ (10.2 mg, 0.0145 mmol) and KF (38.6 mg, 0.670 mmol) in acetonitrile (4 mL) and water (0.4 mL) was heated at 110° C. for 1 h. The reaction mixture was concentrated under reduced pressure, and the resulting residue was partitioned between water and EtOAc. The collected organic layer was dried over anhydrous sodium sulfate and concentrated under vacuum. Purification by flash chromatography on silica gel (gradient: 0-20% methanol/DCM) provided the title compound as a yellow solid (30 mg, 42% yield). LC-MS: (ESI, m/z): [M+H]⁺=728.

### Step 3: 6-((5aS,6S,9R)-3-Chloro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-4-methyl-5-(trifluoromethyl)pyridin-2-amine

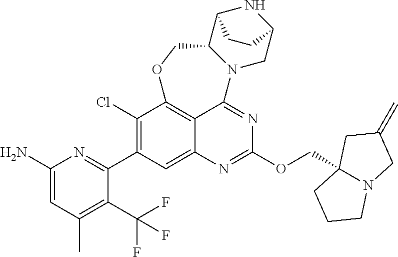 [SMILES: C=C1CN2CCC[C@@]2(COc2nc3c4c(c(Cl)c(-c5nc(N)cc(C)c5C(F)(F)F)cc4n2)OC[C@@H]2[C@@H]4CC[C@H](CN32)N4)C1]

A solution of tert-butyl (5aS,6S,9R)-2-(6-amino-4-methyl-3-(trifluoromethyl)pyridin-2-yl)-3-chloro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate (0.030 g, 0.040 mmol) in TFA (2 mL) and DCM (6 mL) was stirred at room temperature for 1 h. The reaction mixture was concentrated under vacuum. Purified by Prep-HPLC (Column: Xselect CSH OBD Column 30×150 mm 5 μm, n; Mobile Phase A:Water (0.1% FA), Mobile Phase B:ACN; Flow rate:60 mL/min; Gradient:6% B to 29% B in 7 min; R_{T1}:5.93 min) afforded the title compound as a white solid (3.0 mg). LC-MS: (ESI, m/z): [M+H]⁺=628. ¹H NMR (300 MHz, DMSO-d₆, ppm) δ 6.98 (d, J=2.0 Hz, 1H), 6.70 (s, 2H), 6.42 (s, 1H), 4.93 (s, 2H), 4.88-4.70 (m, 1H), 4.70-4.50 (m, 1H), 4.50-4.35 (m, 1H), 4.20-3.95 (m, 3H), 3.83-3.71 (m, 2H), 3.63 (d, J=14.2 Hz, 1H), 3.21-3.00 (m, 3H), 2.65-2.55 (m, 3H), 2.41-2.32 (m, 3H), 2.01-1.60 (m, 8H).

### Example 15: Compound 15: 6-((5aS,6S,9R)-3-Chloro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-5-(trifluoromethyl)pyridin-2-amine

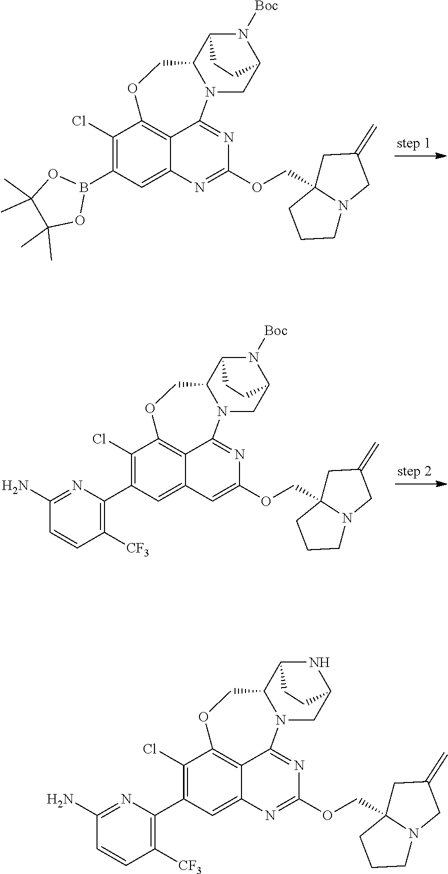

### Step 1: tert-Butyl (5aS,6S,9R)-2-(6-amino-3-(trifluoromethyl)pyridin-2-yl)-3-chloro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate

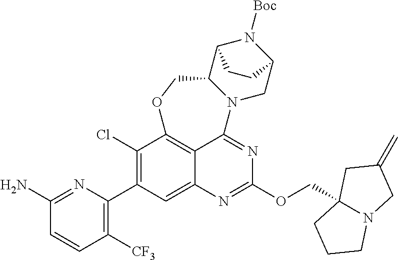 [SMILES: C=C1CN2CCC[C@@]2(COc2nc3c4c(c(Cl)c(-c5nc(N)ccc5C(F)(F)F)cc4n2)OC[C@@H]2[C@@H]4CC[C@H](CN32)N4C(=O)OC(C)(C)C)C1]

A stirred solution of crude tert-butyl (5aS,6S,9R)-3-chloro-13-(((S)-2-methylenetetrahydro-1H-pyrrolizin-7a(5H)-yl)methoxy)-2-(4,4,5,5-tetramethyl-1,3,2-dioxaborolan-2-yl)-5a,6,7,8,9,10-hexahydro-5H-6,9-epiminoazepino[2′,1′:3,4][1,4]oxazepino[5,6,7-de]quinazoline-15-carboxylate (262 mg, Example 14, step 1), 6-bromo-5-(trifluoromethyl)pyridin-2-amine (61.8 mg, 0.260 mmol,), Pd(PPh₃)₂Cl₂ (36.2 mg, 0.0515 mmol), and KF (44.9 mg, 0.770 mmol) in acetonitrile (3 mL) and water (0.6 mL) was heated at 80° C. for 1 h. The reaction mixture was concentrated under vacuum. Purification by pre-packed C18 column (solvent gradient: 0-100% ACN in water (0.05% NH₄HCO₃)) afforded the title compound as a yellow solid (32 mg, 17% yield). LC-MS: (ESI, m/z): [M+H]⁺=714.

### Step 2: 6-((5aS,6S,9R)-3-Chloro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-5-(trifluoromethyl)pyridin-2-amine

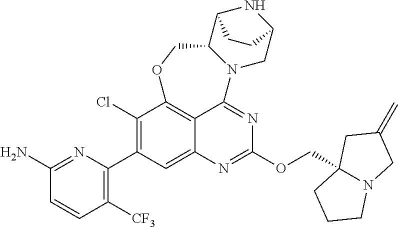 [SMILES: C=C1CN2CCC[C@@]2(COc2nc3c4c(c(Cl)c(-c5nc(N)ccc5C(F)(F)F)cc4n2)OC[C@@H]2[C@@H]4CC[C@H](CN32)N4)C1]

A solution of tert-butyl (5aS,6S,9R)-2-(6-amino-3-(trifluoromethyl)pyridin-2-yl)-3-chloro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate (32.0 mg, 0.0448 mmol) in dichloromethane (2 mL) and 2,2,2-trifluoroacetic acid (1 mL) was stirred at room temperature for 30 min. The reaction was concentrated under vacuum, and the crude product was purified by Prep-HPLC (Column: XBridge Prep OBD C18 Column, 30×150 mm 5 μm; Mobile Phase A:Water (10 mmol/L NH₄HCO₃+0.1% NH₃·H₂O), Mobile Phase B:ACN; Flow rate:60 mL/min; Gradient:24% B to 54% B in 9 min; R_{T1}:8.5 min) afford crude product (6 mg). The material was repurified by Prep-HPLC (XBridge Prep OBD C18 Column, 19×250 mm, 5 μm; Mobile Phase A:Water (0.05% FA), Mobile Phase B:ACN; Gradient:22% B to 38% B in 10 min, R_{T1}:7.82 min) to yield the title compound as a white solid (1.2 mg, 4.4% yield). LC-MS: (ESI, m/z): [M+H]⁺=614. ¹H NMR (300 MHz, DMSO-d₆, ppm) δ 7.76 (dd, J=8.9, 2.8 Hz, 1H), 7.04 (d, J=2.8 Hz, 1H), 6.87 (s, 2H), 6.58 (d, J=8.8 Hz, 1H), 4.93 (s, 2H), 4.79 (dd, J=29.2, 12.8 Hz, 1H), 4.61 (t, J=12.0 Hz, 1H), 4.50-4.30 (m, 1H), 4.16-3.90 (m, 3H), 3.80-3.50 (m, 3H), 3.20-3.00 (m, 3H), 2.70-2.55 (m, 2H), 2.40-2.30 (m, 1H), 2.05-1.55 (m, 9H).

### Example 16: Compound 16A and Compound 16B: (5aS,6S,9R)-3-Chloro-2-(6-fluoro-1-methyl-1H-indazol-7-yl)-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline

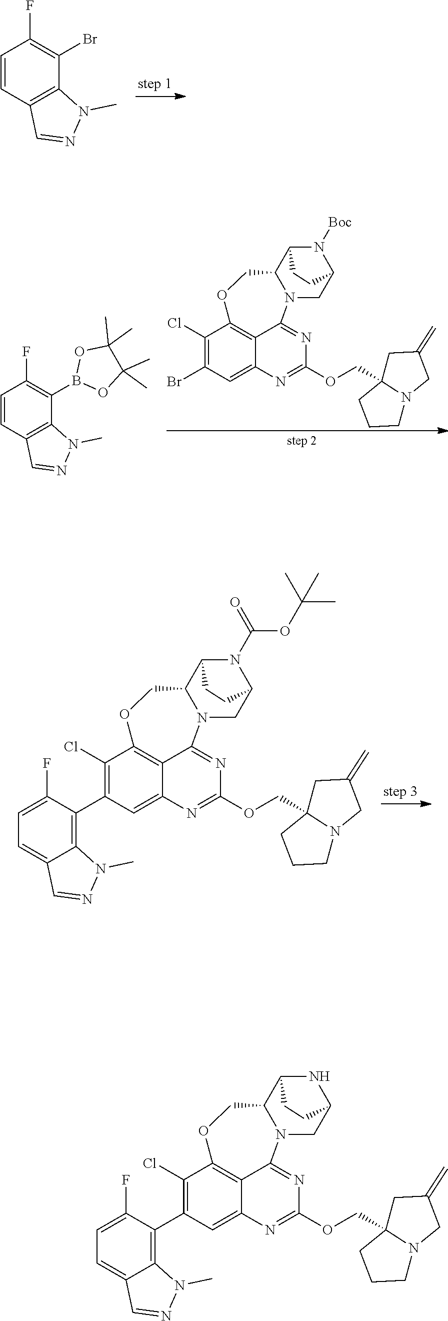

### Step 1: 6-Fluoro-1-methyl-7-(4,4,5,5-tetramethyl-1,3,2-dioxaborolan-2-yl)-1H-indazole

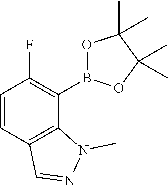 [SMILES: Cn1ncc2ccc(F)c(B3OC(C)(C)C(C)(C)O3)c21]

A stirred solution of 7-bromo-6-fluoro-1-methyl-1H-indazole (1.70 g, 7.42 mmol), B₂Pin₂ (9.47 g, 37.3 mmol), Pd(dppf)Cl₂ (545 mg, 0.750 mmol) and KOAc (3.34 g, 34.1 mmol) in DMF (30 mL) was heated at 80° C. under nitrogen for 12 h. The reaction mixture was diluted with ethyl acetate and washed with water (3×). The organic layer was dried over anhydrous sodium sulfate and concentrated under vacuum. Purification by flash chromatography on silica gel (gradient: 0-3% ethyl acetate/petroleum ether) afforded the title compound as a white solid (1.40 g, 68.3% yield). LC-MS: (ESI, m/z): [M+H]⁺=277.

### Step 2: tert-Butyl (5aS,6S,9R)-3-chloro-2-(6-fluoro-1-methyl-1H-indazol-7-yl)-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate

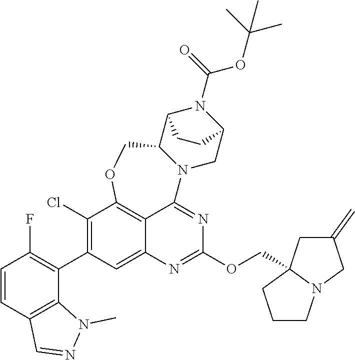 [SMILES: C=C1CN2CCC[C@@]2(COc2nc3c4c(c(Cl)c(-c5c(F)ccc6cnn(C)c56)cc4n2)OC[C@@H]2[C@@H]4CC[C@H](CN32)N4C(=O)OC(C)(C)C)C1]

A stirred solution of tert-butyl (5aS,6S,9R)-2-bromo-3-chloro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate (274 mg, 0.430 mmol, Intermediate 9), 6-fluoro-1-methyl-7-(4,4,5,5-tetramethyl-1,3,2-dioxaborolan-2-yl)-1H-indazole (797 mg, 2.89 mmol), Pd(PPh₃)₂Cl₂ (48.5 mg, 0.0700 mmol) and KF (113 mg, 1.94 mmol) in acetonitrile (15 mL) and water (1.5 mL) under nitrogen was heated for 1.5 h at 90° C. The reaction mixture was concentrated under reduced pressure, and the resulting residue was partitioned between ethyl acetate and water. The organic layer was dried over anhydrous sodium sulfate and concentrated under vacuum. Purification by flash chromatography on silica gel (gradient: 0-5% methanol/DCM) afforded of the title compound as a yellow solid (100 mg, 30% yield). LC-MS: (ESI, m/z): [M+H]⁺=702.

### Step 3: (5aS,6S,9R)-3-Chloro-2-(6-fluoro-1-methyl-1H-indazol-7-yl)-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline (two single unknown atropisomers)

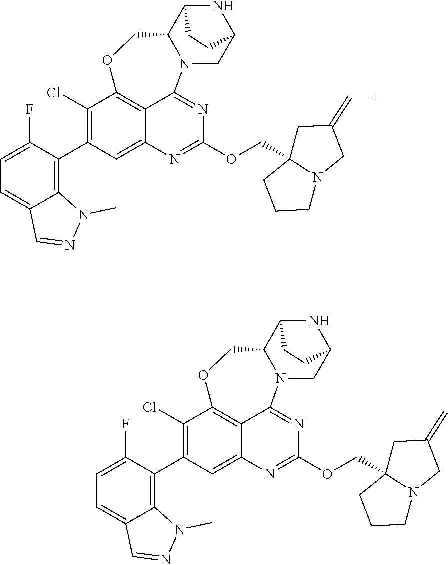 [SMILES: C=C1CN2CCC[C@@]2(COc2nc3c4c(c(Cl)c(-c5c(F)ccc6cnn(C)c56)cc4n2)OC[C@@H]2[C@@H]4CC[C@H](CN32)N4)C1 ; C=C1CN2CCC[C@@]2(COc2nc3c4c(c(Cl)c(-c5c(F)ccc6cnn(C)c56)cc4n2)OC[C@@H]2[C@@H]4CC[C@H](CN32)N4)C1]

A solution of tert-butyl (5aS,6S,9R)-3-chloro-2-(6-fluoro-1-methyl-1H-indazol-7-yl)-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate (23.0 mg, 0.0300 mmol) in 2,2,2-trifluoroacetic acid (1 mL) and DCM (3 mL) was stirred at room temperature for 1 hour. The reaction mixture was concentrated under vacuum. Purification by Prep-HPLC (Column: XBridge Prep OBD C18 Column, 30×150 mm 5 μm; Mobile Phase A:Water (10 mmol/L NH₄HCO₃), Mobile Phase B:ACN; Flow rate:60 mL/min; Gradient:30% B to 60% B in 10 min; 254 nm; RT₁:8.83) yielded a yellow solid (32 mg, mixture of two atropisomers). The atropisomers were separated by Chiral Prep HPLC (Column: CHIRALPAK IA, 2×25 cm, 5 μm; Mobile Phase A:Hex:DCM=3:1 (10 mmol/L NH₃), Mobile Phase B:EtOH; Flow rate:15 mL/min; Gradient:50% B to 50% B in 23 min; R_{T1}:7.084 min; R_{T2}:18.379 min) to yield 10.1 mg (11% yield) of Compound 16A (the faster peak) and 10.0 mg (11% yield) of Compound 16B (the slower peak) as white solids.

Compound 16A: LC-MS: (ESI, m/z): [M+H]⁺=602. ¹H NMR (300 MHz, DMSO-d₆, ppm) δ 8.15 (s, 1H), 7.90 (dd, J=8.8, 5.1 Hz, 1H), 7.31 (s, 1H), 7.16 (t, J=9.2 Hz, 1H), 4.93-4.79 (m, 3H), 4.70-4.60 (m, 1H), 4.47 (dd, J=13.1, 7.7 Hz, 1H), 4.10-3.91 (m, 3H), 3.70-3.50 (m, 6H), 3.25-2.96 (m, 4H), 2.62-2.55 (m, 1H), 2.35-2.30 (m, 1H), 2.03-1.50 (m, 8H).

Compound 16B: LC-MS: (ESI, m/z): [M+H]⁺=602. ¹H NMR (300 MHz, DMSO-d₆, ppm) δ 8.14 (s, 1H), 7.90 (dd, J=8.8, 5.1 Hz, 1H), 7.32 (s, 1H), 7.17 (t, J=9.2 Hz, 1H), 4.86 (d, J=26.4 Hz, 3H), 4.70-4.59 (m, 1H), 4.47 (dd, J=13.1, 7.9 Hz, 1H), 4.12-3.90 (m, 3H), 3.64-3.45 (m, 6H), 3.25-2.93 (m, 4H), 2.63-2.55 (m, 1H), 2.40-2.30 (m, 1H), 1.99-1.56 (m, 8H).

### Example 17: Compound 17A and Compound 17B: (2S,5aS,6S,9R)-3-Chloro-1-fluoro-2-(6-fluoro-1-methyl-1H-indazol-7-yl)-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline

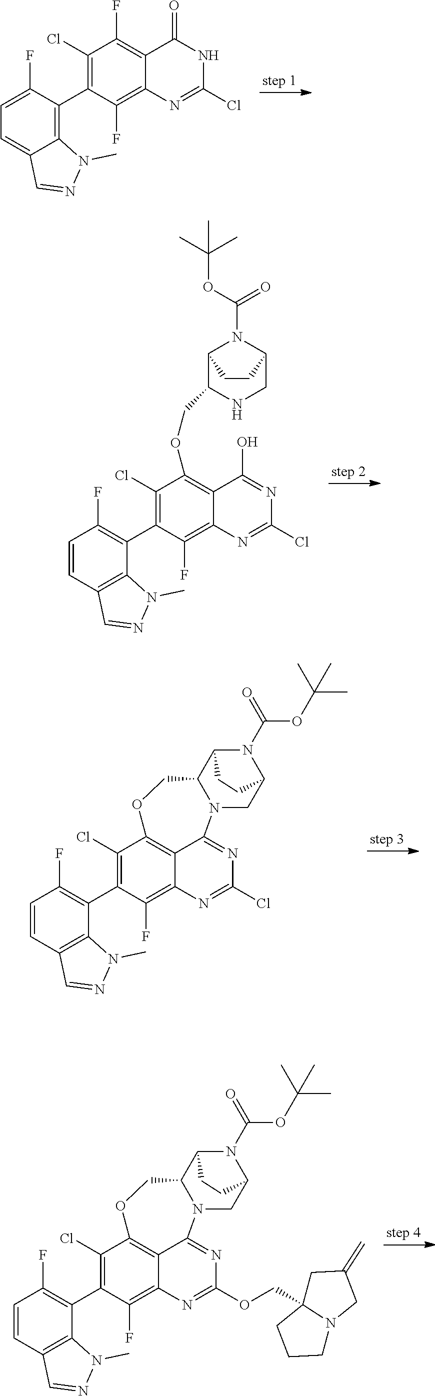 [SMILES: Cn1ncc2ccc(F)c(-c3c(Cl)c(F)c4c(=O)[nH]c(Cl)nc4c3F)c21 ; Cn1ncc2ccc(F)c(-c3c(Cl)c(OC[C@H]4NC[C@H]5CC[C@@H]4N5C(=O)OC(C)(C)C)c4c(O)nc(Cl)nc4c3F)c21 ; Cn1ncc2ccc(F)c(-c3c(Cl)c4c5c(nc(Cl)nc5c3F)N3C[C@H]5CC[C@@H]([C@H]3CO4)N5C(=O)OC(C)(C)C)c21 ; C=C1CN2CCC[C@@]2(COc2nc3c4c(c(Cl)c(-c5c(F)ccc6cnn(C)c56)c(F)c4n2)OC[C@@H]2[C@@H]4CC[C@H](CN32)N4C(=O)OC(C)(C)C)C1]

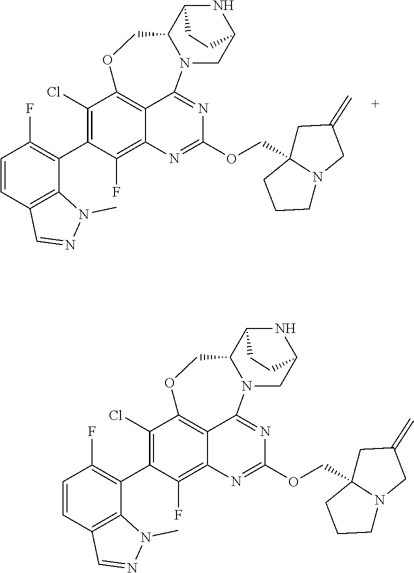 [SMILES: C=C1CN2CCC[C@@]2(COc2nc3c4c(c(Cl)c(-c5c(F)ccc6cnn(C)c56)c(F)c4n2)OC[C@@H]2[C@@H]4CC[C@H](CN32)N4)C1 ; C=C1CN2CCC[C@@]2(COc2nc3c4c(c(Cl)c(-c5c(F)ccc6cnn(C)c56)c(F)c4n2)OC[C@@H]2[C@@H]4CC[C@H](CN32)N4)C1]

### Step 1: tert-Butyl (1R,2S,5S)-2-(((2,6-dichloro-8-fluoro-7-(6-fluoro-1-methyl-1H-indazol-7-yl)-4-hydroxyquinazolin-5-yl)oxy)methyl)-3,8-diazabicyclo[3.2.1]octane-8-carboxylate

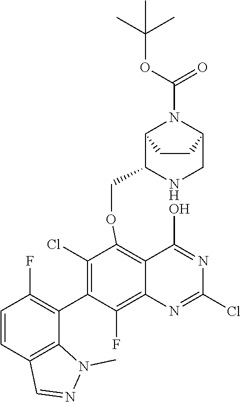 [SMILES: Cn1ncc2ccc(F)c(-c3c(Cl)c(OC[C@H]4NC[C@H]5CC[C@@H]4N5C(=O)OC(C)(C)C)c4c(O)nc(Cl)nc4c3F)c21]

To an ice-cooled solution of tert-butyl (1S,2S,5R)-2-(hydroxymethyl)-3,8-diazabicyclo[3.2.1]octane-8-carboxylate (195 mg, 0.800 mmol, Intermediate 1) in tetrahydrofuran (5 mL) was added NaH (128 mg, 3.20 mmol, 60% in mineral oil). The resulting mixture was warmed to room temperature for 30 min. 2,6-Dichloro-5,8-difluoro-7-(6-fluoro-1-methyl-1H-indazol-7-yl)quinazolin-4 (3H)-one (320 mg, 0.80 mmol, Intermediate 10) was added at room temperature. After 1 h, the reaction was diluted with H₂O, and the mixture was extracted with EtOAc. The combined organics were dried over anhydrous sodium sulfate, filtered, and concentrated. Purification by flash chromatography on silica gel (gradient: 0-20% methanol/DCM) afforded a white solid (363 mg, 72% yield). LC-MS: (ESI, m/z): [M+H]⁺=621.

### Step 2: tert-Butyl (5aS,6S,9R)-3,13-dichloro-1-fluoro-2-(6-fluoro-1-methyl-1H-indazol-7-yl)-5a,6,7,8,9,10-hexahydro-5H-6,9-epiminoazepino[2′,1′:3,4][1,4]oxazepino[5,6,7-de]quinazoline-15-carboxylate

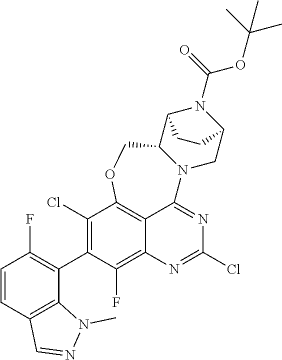 [SMILES: Cn1ncc2ccc(F)c(-c3c(Cl)c4c5c(nc(Cl)nc5c3F)N3C[C@H]5CC[C@@H]([C@H]3CO4)N5C(=O)OC(C)(C)C)c21]

Under nitrogen, a solution of tert-butyl (1R,2S,5S)-2-(((2,6-dichloro-8-fluoro-7-(6-fluoro-1-methyl-1H-indazol-7-yl)-4-hydroxyquinazolin-5-yl)oxy)methyl)-3,8-diazabicyclo[3.2.1]octane-8-carboxylate (360 mg, 0.58 mmol), BOPCl (441 mg, 1.73 mmol) and DIPEA (747 mg, 5.79 mmol) in 1,2-dichloroethane (2 mL) was stirred at room temperature for 2 h. The reaction mixture was diluted with EtOAc and washed with H₂O. The collected organic was washed with brine, dried over anhydrous sodium sulfate, filtered, and concentrated under vacuum. Purification by flash chromatography on silica gel (gradient: 0-20% methanol/DCM) provided the title compound (180 mg, 51% yield) as a yellow solid. LC-MS: (ESI, m/z): [M+H]⁺=603.

### Step 3: tert-Butyl (5aS,6S,9R)-3-chloro-1-fluoro-2-(6-fluoro-1-methyl-1H-indazol-7-yl)-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate

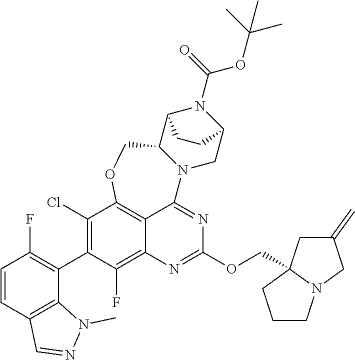 [SMILES: C=C1CN2CCC[C@@]2(COc2nc3c4c(c(Cl)c(-c5c(F)ccc6cnn(C)c56)c(F)c4n2)OC[C@@H]2[C@@H]4CC[C@H](CN32)N4C(=O)OC(C)(C)C)C1]

To an ice-cooled solution of (S)-(2-methylenetetrahydro-1H-pyrrolizin-7a(5H)-yl)methanol (54.0 mg, 0.350 mmol, Intermediate 15) in tetrahydrofuran (2 mL) under nitrogen was added NaH (36.0 mg, 0.900 mmol, 60% in mineral oil). The resulting solution was warmed to room temperature. After 30 min, tert-butyl (5aS,6S,9R)-3,13-dichloro-1-fluoro-2-(6-fluoro-1-methyl-1H-indazol-7-yl)-5a,6,7,8,9,10-hexahydro-5H-6,9-epiminoazepino[2′,1′:3,4][1,4]oxazepino[5,6,7-de]quinazoline-15-carboxylate (180 mg, 0.30 mmol) was added, and the reaction was maintained at room temperature for 1 h. The reaction was diluted with H₂O, and the resulting mixture was extracted with EtOAc. The collected organic was dried over anhydrous sodium sulfate, filtered, and concentrated under vacuum. Purification by flash chromatography on silica gel (gradient: 0-20% methanol/DCM) afforded the title compound as a yellow solid (130 mg, 62% yield) of. LC-MS: (ESI, m/z): [M+H]⁺=720.

### Step 4: (2S,5aS,6S,9R)-3-Chloro-1-fluoro-2-(6-fluoro-1-methyl-1H-indazol-7-yl)-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline

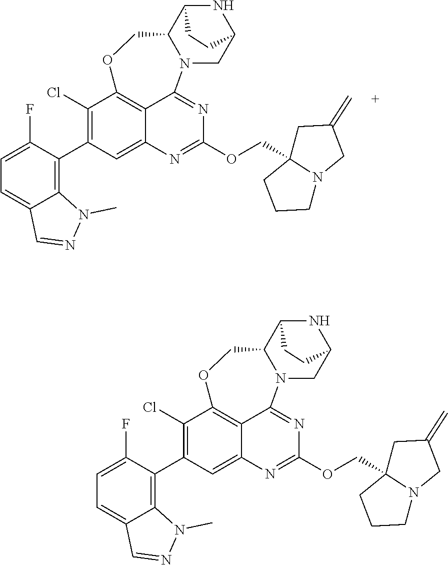 [SMILES: C=C1CN2CCC[C@@]2(COc2nc3c4c(c(Cl)c(-c5c(F)ccc6cnn(C)c56)cc4n2)OC[C@@H]2[C@@H]4CC[C@H](CN32)N4)C1 ; C=C1CN2CCC[C@@]2(COc2nc3c4c(c(Cl)c(-c5c(F)ccc6cnn(C)c56)cc4n2)OC[C@@H]2[C@@H]4CC[C@H](CN32)N4)C1]

A solution of tert-butyl (5aS,6S,9R)-3-chloro-1-fluoro-2-(6-fluoro-1-methyl-1H-indazol-7-yl)-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate (130 mg, 0.18 mmol) in 2,2,2-trifluoroacetic acid (2 mL) and DCM (2 mL) was stirred at room temperature for 1 h. The solvent was concentrated under vacuum. The crude product was purified by Prep-HPLC (Column: XBridge Prep OBD C18 Column, 30×150 mm 5 μm; Mobile Phase A: Water (10 mmol/L NH₄HCO₃), Mobile Phase B: ACN; Flow rate: 60 mL/min; Gradient: 32% B to 62% B in 9 min; 254 nm; RT: 8.00 min.) to yield a white solid (80 mg, mixture of two atropisomer). The atropisomers were separated by Chiral Prep HPLC(Column: CHIRALPAK IE, 2×25 cm 5 μm; Mobile Phase A: Hex:DCM=3:1 (10 mmol NH₃), Mobile Phase B: EtOH; Flow rate: 20 mL/min; Gradient: 50% B to 50% B in 18 min; R_{T1}: 8.956 min. R_{T2}:14.907 min) to yield 25 mg Compound 17A (faster peak) and 25 mg Compound 17B (slower peak) as white solids.

Compound 17A: LC-MS: (ESI, m/z): [M+H]⁺=620. ¹H NMR (400 MHz, DMSO-d₆, ppm) δ 8.19 (s, 1H), 7.98 (dd, J=8.8, 5.2 Hz, 1H), 7.22 (dd, J=9.7, 8.8 Hz, 1H), 5.10-4.80 (m, 3H), 4.69-4.59 (m, 1H), 4.44 (dd, J=13.0, 8.1 Hz, 1H), 4.08-3.91 (m, 3H), 3.62-3.43 (m, 6H), 3.19 (d, J=14.0 Hz, 1H), 3.08 (d, J=12.9 Hz, 1H), 3.01-2.92 (m, 1H), 2.82 (br, 1H), 2.65-2.53 (m, 2H), 2.35 (d, J=15.5 Hz, 1H), 2.03-1.92 (m, 1H), 1.92-1.62 (m, 6H), 1.60-1.50 (m, 1H)

Compound 17B: LC-MS: (ESI, m/z): [M+H]⁺=620. ¹H NMR (400 MHz, DMSO-d₆, ppm) δ 8.18 (s, 1H), 7.98 (dd, J=8.8, 5.2 Hz, 1H), 7.23 (dd, J=9.8, 8.8 Hz, 1H), 4.95-4.82 (m, 3H), 4.70-4.60 (m, 1H), 4.45 (dd, J=13.0, 8.3 Hz, 1H), 4.09 (d, J=8.1 Hz, 1H), 3.98 (q, J=10.4 Hz, 2H), 3.64-3.43 (m, 6H), 3.19 (d, J=14.1 Hz, 1H), 3.07 (d, J=12.8 Hz, 1H), 3.03-2.92 (m, 1H), 2.83 (br, 1H), 2.62-2.53 (m, 2H), 2.35 (d, J=15.5 Hz, 1H), 1.97 (dd, J=11.5, 6.3 Hz, 1H), 1.91-1.61 (m, 6H), 1.60-1.45 (m, 1H)

### Example 18: Compound 18A and Compound 18B: tert-Butyl (5aS,6S,9R)-2-bromo-13-chloro-1,3-difluoro-5a,6,7,8,9,10-hexahydro-5H-6,9-epiminoazepino[2′,1′:3,4][1,4]oxazepino[5,6,7-de]quinazoline-15-carboxylate

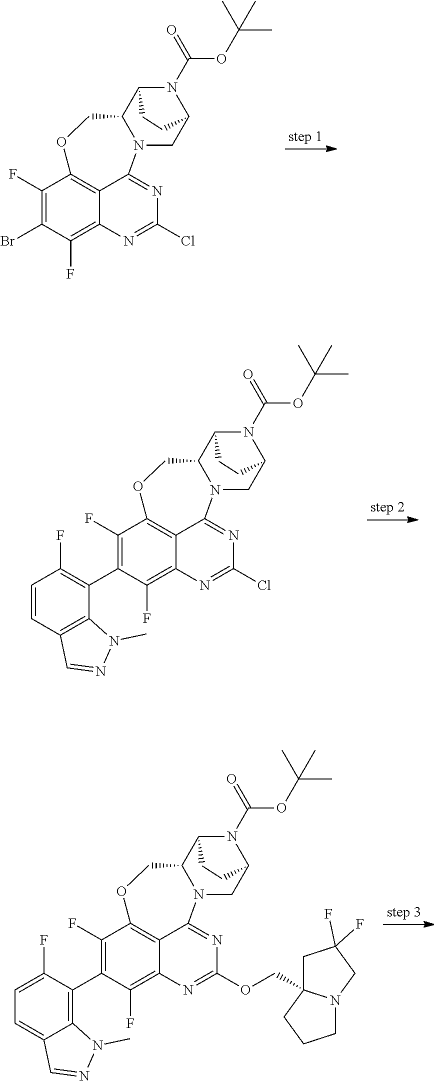 [SMILES: CC(C)(C)OC(=O)N1[C@@H]2CC[C@H]1[C@H]1COc3c(F)c(Br)c(F)c4nc(Cl)nc(c34)N1C2 ; Cn1ncc2ccc(F)c(-c3c(F)c4c5c(nc(Cl)nc5c3F)N3C[C@H]5CC[C@@H]([C@H]3CO4)N5C(=O)OC(C)(C)C)c21 ; Cn1ncc2ccc(F)c(-c3c(F)c4c5c(nc(OC[C@@]67CCCN6CC(F)(F)C7)nc5c3F)N3C[C@H]5CC[C@@H]([C@H]3CO4)N5C(=O)OC(C)(C)C)c21]

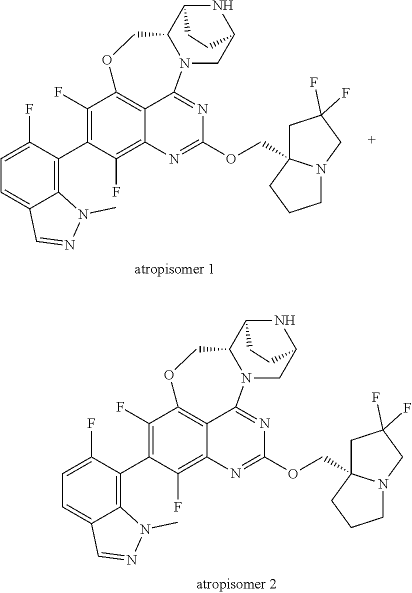 [SMILES: Cn1ncc2ccc(F)c(-c3c(F)c4c5c(nc(OC[C@@]67CCCN6CC(F)(F)C7)nc5c3F)N3C[C@H]5CC[C@H](N5)[C@H]3CO4)c21 ; Cn1ncc2ccc(F)c(-c3c(F)c4c5c(nc(OC[C@@]67CCCN6CC(F)(F)C7)nc5c3F)N3C[C@H]5CC[C@H](N5)[C@H]3CO4)c21]

### Step 1 tert-Butyl (5aS,6S,9R)-13-chloro-1,3-difluoro-2-(6-fluoro-1-methyl-1H-indazol-7-yl)-5a,6,7,8,9,10-hexahydro-5H-6,9-epiminoazepino[2′,1′:3,4][1,4]oxazepino[5,6,7-de]quinazoline-15-carboxylate

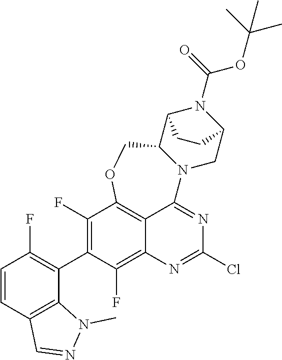 [SMILES: Cn1ncc2ccc(F)c(-c3c(F)c4c5c(nc(Cl)nc5c3F)N3C[C@H]5CC[C@@H]([C@H]3CO4)N5C(=O)OC(C)(C)C)c21]

Under nitrogen, to a solution of tert-butyl (5aS,6S,9R)-13-chloro-1,3-difluoro-2-(6-fluoro-1-methyl-1H-indazol-7-yl)-5a,6,7,8,9,10-hexahydro-5H-6,9-epiminoazepino[2′,1′:3,4][1,4]oxazepino[5,6,7-de]quinazoline-15-carboxylate (180 mg, 0.35 mmol, Intermediate 7) in THF (2 mL) was added 1.3 M iPrMgCl·LiCl in THF (0.31 mL, 0.40 mmol) at −78° C. After 40 min, 2M ZnCl₂ in 2-methyltetrahydrofuran (0.21 mL, 0.42 mmol) was added at −78° C. After 15 min, the reaction mixture was warmed to room temperature for 15 min. 7-Bromo-6-fluoro-1-methyl-indazole (64.0 mg, 0.280 mmol) and (SP-4-1)-[1,3-bis[2,6-bis(1-ethylpropyl)phenyl]-4,5-dichloro-1,3-dihydro-2H-imidazol-2-ylidene]dichloro(2-methylpyridine)palladium (29.0 mg, 0.0345 mmol) in THE (0.8 mL) were added, and the reaction was heated to 50° C. overnight. The reaction was concentrated, and the crude was purified by flash chromatography on silica gel (gradient: 0-70% EtOAc/petroleum ether) to afford crude product (100 mg) which was further purified by pre-packed C18 column (solvent gradient: 0-100% ACN in water (0.05% NH₄HCO₃)) to afford the title compound as a white solid (37 mg, 18% yield). LC-MS: (ESI, m/z): [M+H]⁺=587.

### Step 2: tert-Butyl (5aS,6S,9R)-13-(((S)-2,2-difluorotetrahydro-1H-pyrrolizin-7a(5H)-yl)methoxy)-1,3-difluoro-2-(6-fluoro-1-methyl-1H-indazol-7-yl)-5a,6,7,8,9,10-hexahydro-5H-6,9-epiminoazepino[2′,1′:3,4][1,4]oxazepino[5,6,7-de]quinazoline-15-carboxylate

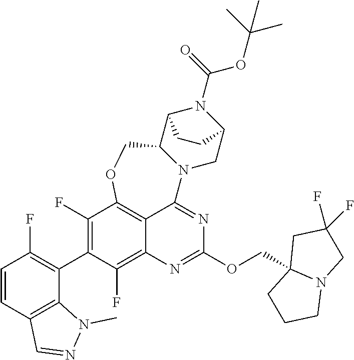 [SMILES: Cn1ncc2ccc(F)c(-c3c(F)c4c5c(nc(OC[C@@]67CCCN6CC(F)(F)C7)nc5c3F)N3C[C@H]5CC[C@@H]([C@H]3CO4)N5C(=O)OC(C)(C)C)c21]

To a solution of (S)-(2,2-difluorotetrahydro-1H-pyrrolizin-7a(5H)-yl)methanol (12.2 mg, 0.0689 mmol, Intermediate 16) in tetrahydrofuran (2 mL) was added NaH (8.3 mg, 0.21 mmol, 60% in mineral oil). The reaction was stirred at room temperature for 15 min before the addition of tert-butyl (5aS,6S,9R)-13-chloro-1,3-difluoro-2-(6-fluoro-1-methyl-1H-indazol-7-yl)-5a,6,7,8,9,10-hexahydro-5H-6,9-epiminoazepino[2′,1′:3,4][1,4]oxazepino[5,6,7-de]quinazoline-15-carboxylate (27.0 mg, 0.0459 mmol). The reaction mixture was heated to 40° C. for 1 h. The reaction was quenched with AcOH, diluted with water, and extracted with DCM. The combined organic layers were dried over anhydrous Na₂SO₄ and concentrated to afford of the crude title compound as a yellow oil (30 mg). LC-MS: (ESI, m/z): [M+H]⁺=728. The crude product was used without further purification.

### Step 3: (5aS,6S,9R)-13-(((S)-2,2-Difluorotetrahydro-1H-pyrrolizin-7a(5H)-yl)methoxy)-1,3-difluoro-2-(6-fluoro-1-methyl-1H-indazol-7-yl)-5a,6,7,8,9,10-hexahydro-5H-6,9-epiminoazepino[2′,1′:3,4][1,4]oxazepino[5,6,7-de]quinazoline

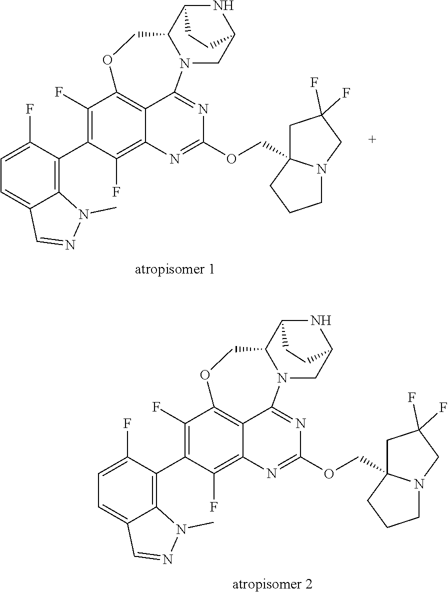 [SMILES: Cn1ncc2ccc(F)c(-c3c(F)c4c5c(nc(OC[C@@]67CCCN6CC(F)(F)C7)nc5c3F)N3C[C@H]5CC[C@H](N5)[C@H]3CO4)c21 ; Cn1ncc2ccc(F)c(-c3c(F)c4c5c(nc(OC[C@@]67CCCN6CC(F)(F)C7)nc5c3F)N3C[C@H]5CC[C@H](N5)[C@H]3CO4)c21]

A solution of tert-butyl (5aS,6S,9R)-13-(((S)-2,2-difluorotetrahydro-1H-pyrrolizin-7a(5H)-yl)methoxy)-1,3-difluoro-2-(6-fluoro-1-methyl-1H-indazol-7-yl)-5a,6,7,8,9,10-hexahydro-5H-6,9-epiminoazepino[2′,1′:3,4][1,4]oxazepino[5,6,7-de]quinazoline-15-carboxylate (30 mg, crude) in TFA (1 mL) and DCM (1 mL) was stirred at room temperature for 30 min. The solvent was concentrated under vacuum. Purification by Prep HPLC (Column: XBridge Prep OBD C18 Column, 19×250 mm, 5 μm; Mobile Phase A:Water (10 mmol/L NH₄HCO₃), Mobile Phase B:MeOH; Gradient:69% B to 72% B in 9 min, R_{T1}:6.33 min; R_{T2}: 7.9 min) afforded Compound 18A (faster peak, 4.8 mg, 11% yield) and Compound 18B (slower peak, 4.3 mg, 10% yield) as white solids.

Compound 18A: LC-MS: (ESI, m/z): [M+H]⁺=628. ¹H NMR (300 MHz, DMSO-d₆, ppm) δ 8.20 (s, 1H), 8.01 (dd, J=8.8, 5.2 Hz, 1H), 7.25 (t, J=9.3 Hz, 1H), 4.86 (dd, J=13.0, 2.4 Hz, 1H), 4.62 (dd, J=13.1, 2.6 Hz, 1H), 4.43 (dd, J=13.0, 8.0 Hz, 1H), 4.20-4.01 (m, 3H), 3.90-3.70 (m, 2H), 3.63 (s, 3H), 3.30-2.97 (m, 4H), 2.78-2.60 (m, 1H), 2.43-2.26 (m, 2H), 2.09-1.60 (m, 8H)

Compound 18B: LC-MS: (ESI, m/z): [M+H]⁺=628. ¹H NMR (300 MHz, DMSO-d₆, ppm) δ 8.20 (s, 1H), 8.01 (dd, J=8.8, 5.2 Hz, 1H), 7.25 (t, J=9.3 Hz, 1H), 4.86 (dd, J=13.0, 2.4 Hz, 1H), 4.62 (dd, J=13.1, 2.6 Hz, 1H), 4.43 (dd, J=13.0, 8.0 Hz, 1H), 4.20-4.01 (m, 3H), 3.70-3.50 (m, 6H), 3.23-2.98 (m, 3H), 2.80-2.68 (m, 1H), 2.43-2.25 (m, 2H), 2.10-1.50 (m, 8H)

### Example 19: Compound 19: 6-((5aS,6S,9R)-1-Fluoro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-4-methyl-5-(trifluoromethyl)pyridin-2-amine

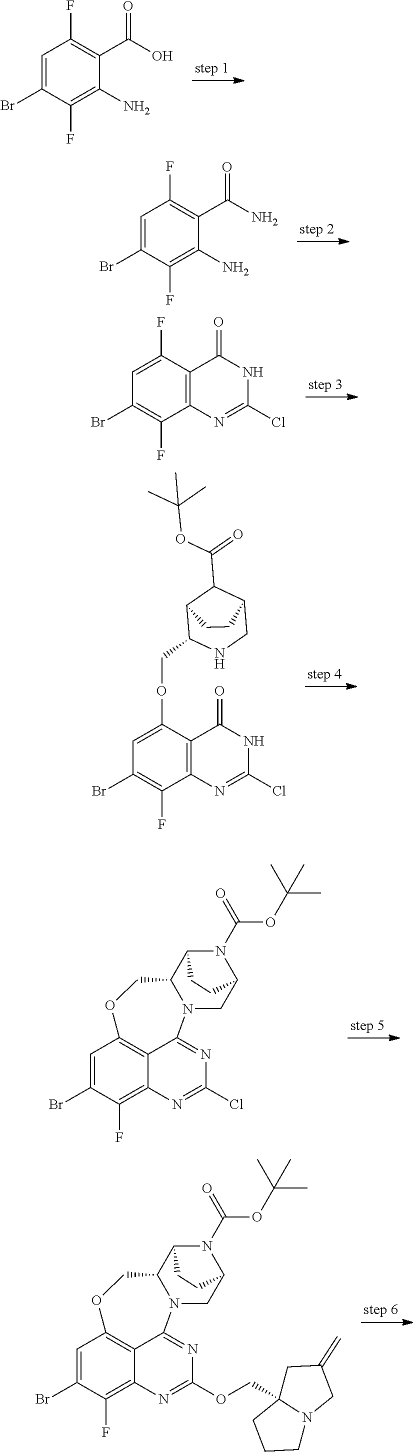

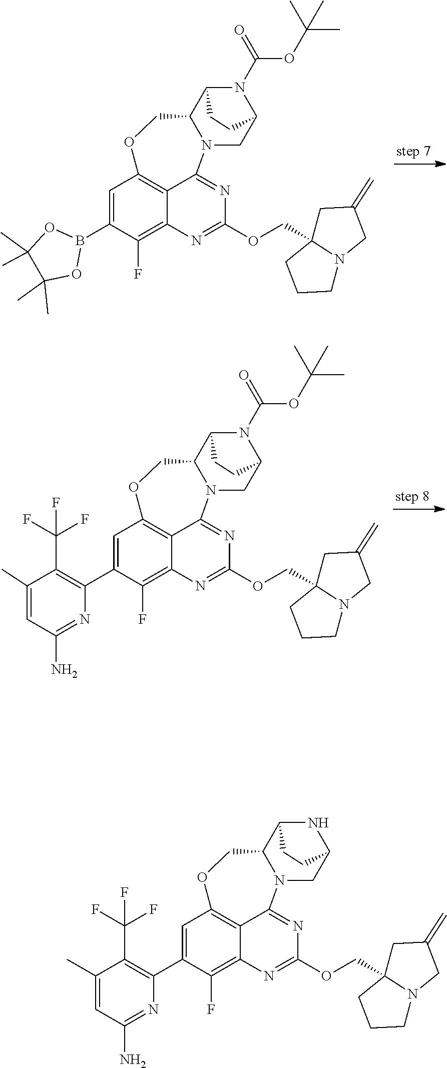

### Step 1: 2-Amino-4-bromo-3,6-difluorobenzamide

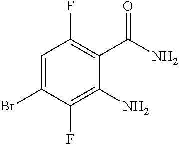 [SMILES: NC(=O)c1c(F)cc(Br)c(F)c1N]

To an ice-cooled solution of 2-amino-4-bromo-3,6-difluorobenzoic acid (5.00 g, 19.8 mmol, Intermediate 2, step 4), DIPEA (15.0 g, 119 mmol), and NH₄Cl (3.20 g, 59.8 mmol) in DMF (50 mL) was added HATU (11.3 g, 29.7 mmol) in portions. The resulting solution was warmed to room temperature for 2 h. The reaction mixture was poured into water and extracted with EtOAc. The collected organic was washed with brine, dried over anhydrous Na₂SO₄, filtered, and concentrated. The crude material was suspended in DCM (30 mL) and filtered to afford the title compound (3.20 g, crude) as a white solid. LC-MS: (ESI, m/z): [M+H]⁺=251.

### Step 2: 7-Bromo-2-chloro-5,8-difluoroquinazolin-4 (3H)-one

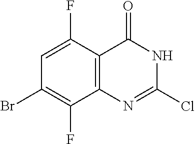 [SMILES: O=c1[nH]c(Cl)nc2c(F)c(Br)cc(F)c12]

To an ice-cooled solution of 2-amino-4-bromo-3,6-difluorobenzamide (3.20 g, 12.7 mmol) in DMF (40 ml) was added thiophosgene (3.08 g, 26.7 mmol). The reaction mixture was heated to 105° C. for 1 h. The reaction was cooled to room temperature and concentrated to a volume of ˜20 mL. Methyl tert-butyl ether (20 mL) was added, and the suspension was stirred at 0° C. for 20 min. The solids were collected by filtration and dried to afford the title compound as a yellow solid (1.72 g, 38% yield). LC-MS: (ESI, m/z): [M+H]⁺=295.

### Step 3: tert-Butyl (1R,2S,5S)-2-(((7-bromo-2-chloro-8-fluoro-4-oxo-3,4-dihydroquinazolin-5-yl)oxy)methyl)-3,8-diazabicyclo[3.2.1]octane-8-carboxylate

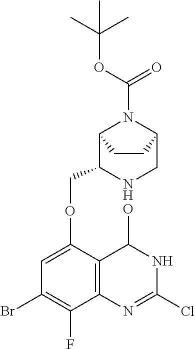 [SMILES: CC(C)(C)OC(=O)N1[C@@H]2CC[C@H]1[C@@H](COc1cc(Br)c(F)c3c1C(O)NC(Cl)=N3)NC2]

To an ice-cooled solution of tert-butyl rac-(1 S,2S,5R)-2-(hydroxymethyl)-3,8-diazabicyclo[3.2.1]octane-8-carboxylate (517 mg, 2.14 mmol, Intermediate 1) in tetrahydrofuran (7 mL) was added NaH (284 mg, 7.12 mmol, 60% in mineral oil). The resulting solution was warmed to room temperature. After 30 min, 7-bromo-2-chloro-5,8-difluoroquinazolin-4 (3H)-one (698 mg, 2.36 mmol) was added, and the reaction was maintained at room temperature for 3 h. The reaction was quenched with H₂O and extracted with EtOAc. The combined organics were dried over anhydrous Na₂SO₄, filtered, and concentrated. The residue was purified by pre-packed C18 column (solvent gradient: 0-100% ACN in water (0.05% NH₄HCO₃)) to afford the title compound as a white solid (232 mg, 19% yield). LC-MS: (ESI, m/z): [M+H]⁺=517.

### Step 4: tert-Butyl (5aS,6S,9R)-2-bromo-13-chloro-1-fluoro-5a,6,7,8,9,10-hexahydro-5H-6,9-epiminoazepino[2′,1′:3,4][1,4]oxazepino[5,6,7-de]quinazoline-15-carboxylate

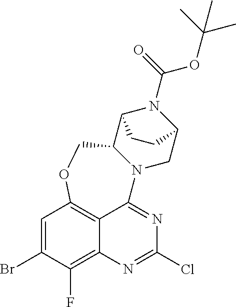 [SMILES: CC(C)(C)OC(=O)N1[C@@H]2CC[C@H]1[C@H]1COc3cc(Br)c(F)c4nc(Cl)nc(c34)N1C2]

Under nitrogen, a stirred solution of tert-butyl (1R,2S,5S)-2-(((7-bromo-2-chloro-8-fluoro-4-oxo-3,4-dihydroquinazolin-5-yl)oxy)methyl)-3,8-diazabicyclo[3.2.1]octane-8-carboxylate (230 mg, 0.44 mmol), BOPCl (340 mg, 1.3 mmol) and DIPEA (576 mg, 4.47 mmol) in 1,2-dichloroethane (3 mL) was heated at 60° C. for 1 h. The reaction mixture was diluted with DCM and washed with water. The organic was dried over anhydrous sodium sulfate, filtered, and concentrated. Purification by flash chromatography on silica gel (gradient: 0-25% EtOAc/petroleum ether) afforded the title compound as a yellow solid (116 mg, 52% yield). LC-MS: (ESI, m/z): [M+H]⁺=499.

### Step 5: tert-Butyl (5aS,6S,9R)-2-bromo-1-fluoro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate

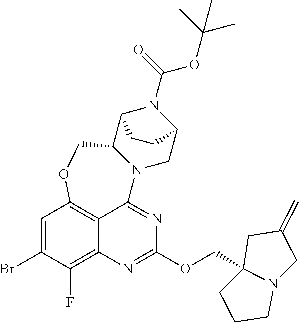 [SMILES: C=C1CN2CCC[C@@]2(COc2nc3c4c(cc(Br)c(F)c4n2)OC[C@@H]2[C@@H]4CC[C@H](CN32)N4C(=O)OC(C)(C)C)C1]

To an ice-cooled solution of (S)-(2-methylenetetrahydro-1H-pyrrolizin-7a(5H)-yl)methanol (42.8 mg, 0.280 mmol, Intermediate 15) in tetrahydrofuran (2.5 mL) under nitrogen was added NaH (27.9 mg, 0.700 mmol, 60% in mineral oil). The resulting solution was warmed to room temperature. After 30 min, tert-butyl (5aS,6S,9R)-2-bromo-13-chloro-1-fluoro-5a,6,7,8,9,10-hexahydro-5H-6,9-epiminoazepino[2′,1′:3,4][1,4]oxazepino[5,6,7-de]quinazoline-15-carboxylate (116 mg, 0.230 mmol) was added, and the resulting mixture was heated at 40° C. for 1 h. The reaction was quenched with H₂O and extracted with EtOAc. The combined organics were dried over anhydrous sodium sulfate, filtered, and concentrated. Purification by flash chromatography on silica gel (gradient: 0-100% EtOAc/petroleum ether) afforded the title compound as a white solid (110 mg, 76% yield). LC-MS: (ESI, m/z): [M+H]⁺=616.

### Step 6: tert-Butyl (5aS,6S,9R)-1-fluoro-13-(((S)-2-methylenetetrahydro-1H-pyrrolizin-7a(5H)-yl)methoxy)-2-(4,4,5,5-tetramethyl-1,3,2-dioxaborolan-2-yl)-5a,6,7,8,9,10-hexahydro-5H-6,9-epiminoazepino[2′,1′:3,4][1,4]oxazepino[5,6,7-de]quinazoline-15-carboxylate

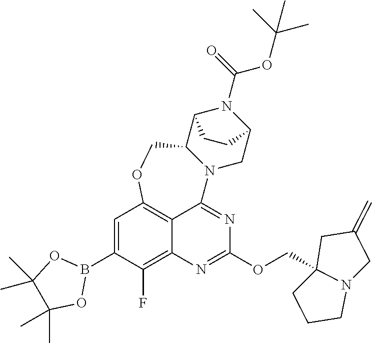

Under nitrogen, a stirred solution of tert-Butyl (5aS,6S,9R)-2-bromo-1-fluoro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate (110 mg, 0.18 mmol), B₂Pin₂ (132 mg, 0.520 mmol), Pd(dppf)Cl₂ (26.2 mg, 0.0400 mmol) and KOAc (52.6 mg, 0.54 mmol) in 1,4-dioxane (2.5 mL) was heated at 80° C. After 3 h, the reaction mixture was concentrated, and the resulting residue was suspended with a solution of EtOAc: petroleum ether (1:10) and stirred at room temperature for 0.5 h. The solids filtered, and the filtrate was concentrated under vacuum to afford the crude title compound as a yellow solid (125 mg). LC-MS: (ESI, m/z): [M+H]⁺=582 (boronic acid).

### Step 7: tert-Butyl (5aS,6S,9R)-2-(6-amino-4-methyl-3-(trifluoromethyl)pyridin-2-yl)-1-fluoro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate

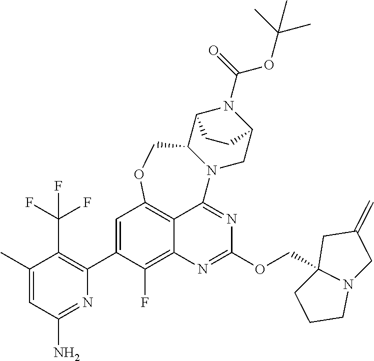

Under nitrogen, a stirred solution of tert-butyl (5aS,6S,9R)-1-fluoro-13-(((S)-2-methylenetetrahydro-1H-pyrrolizin-7a(5H)-yl)methoxy)-2-(4,4,5,5-tetramethyl-1,3,2-dioxaborolan-2-yl)-5a,6,7,8,9,10-hexahydro-5H-6,9-epiminoazepino[2′,1′:3,4][1,4]oxazepino[5,6,7-de]quinazoline-15-carboxylate (124 mg), 6-bromo-4-methyl-5-(trifluoromethyl)pyridin-2-amine (33.0 mg, 0.130 mmol), Pd(PPh₃)Cl₂ (18.2 mg, 0.0300 mmol) and KF (22.6 mg, 0.390 mmol) in acetonitrile (2 mL) and H₂O (0.4 mL) was heated at 80° C. for 1 h. The solvent was removed under vacuum, and the resulting residue was purified by pre-packed C18 column (solvent gradient: 0-100% ACN in water (0.05% NH₄HCO₃)) to afford the title compound as a white solid (18.0 mg, 19% yield) of. LC-MS: (ESI, m/z): [M+H]⁺=712.

### Step 8: 6-((5aS,6S,9R)-1-Fluoro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-4-methyl-5-(trifluoromethyl)pyridin-2-amine

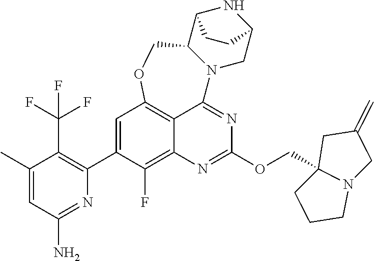

A solution of tert-butyl (5aS,6S,9R)-2-(6-amino-4-methyl-3-(trifluoromethyl)pyridin-2-yl)-1-fluoro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate (18.0 mg, 0.0252 mmol) in dichloromethane (1 mL) and 2,2,2-trifluoroacetic acid (0.5 mL) was stirred at room temperature for 30 min. The solvent was removed under vacuum, and the crude product was purified by Prep HPLC (Column: XBridge Prep OBD C18 Column, 30×150 mm 5 μm; Mobile Phase A:Water (10 mmol/L NH₄HCO₃), Mobile Phase B:ACN; Flow rate:60 mL/min; Gradient:26% B to 56% B in 9 min; 254 nm; R_{T1}:8.5 min) to yield the title compound as a white solid (3.9 mg, 25% yield). LC-MS: (ESI, m/z): [M+H]⁺=612. ¹H NMR (300 MHz, DMSO-d₆, ppm) δ6.79 (s, 2H), 6.61 (dd, J=6.1, 2.4 Hz, 1H), 6.45 (s, 1H), 4.95-4.73 (m, 3H), 4.50-4.35 (m, 1H), 4.28-4.20 (m, 1H), 4.04-3.88 (m, 3H), 3.56 (d, J=13.7 Hz, 2H), 3.44 (s, 1H), 3.19 (d, J=14.0 Hz, 1H), 3.10-2.95 (m, 2H), 2.65-2.55 (m, 2H), 2.42-2.29 (m, 4H), 2.03-1.47 (m, 8H).

### Example 20: Compound 20A and Compound 20B: 5-Chloro-6-((5aS,6S,9R)-3-chloro-1-fluoro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-4-methylpyridin-2-amine (two single artroisomers)

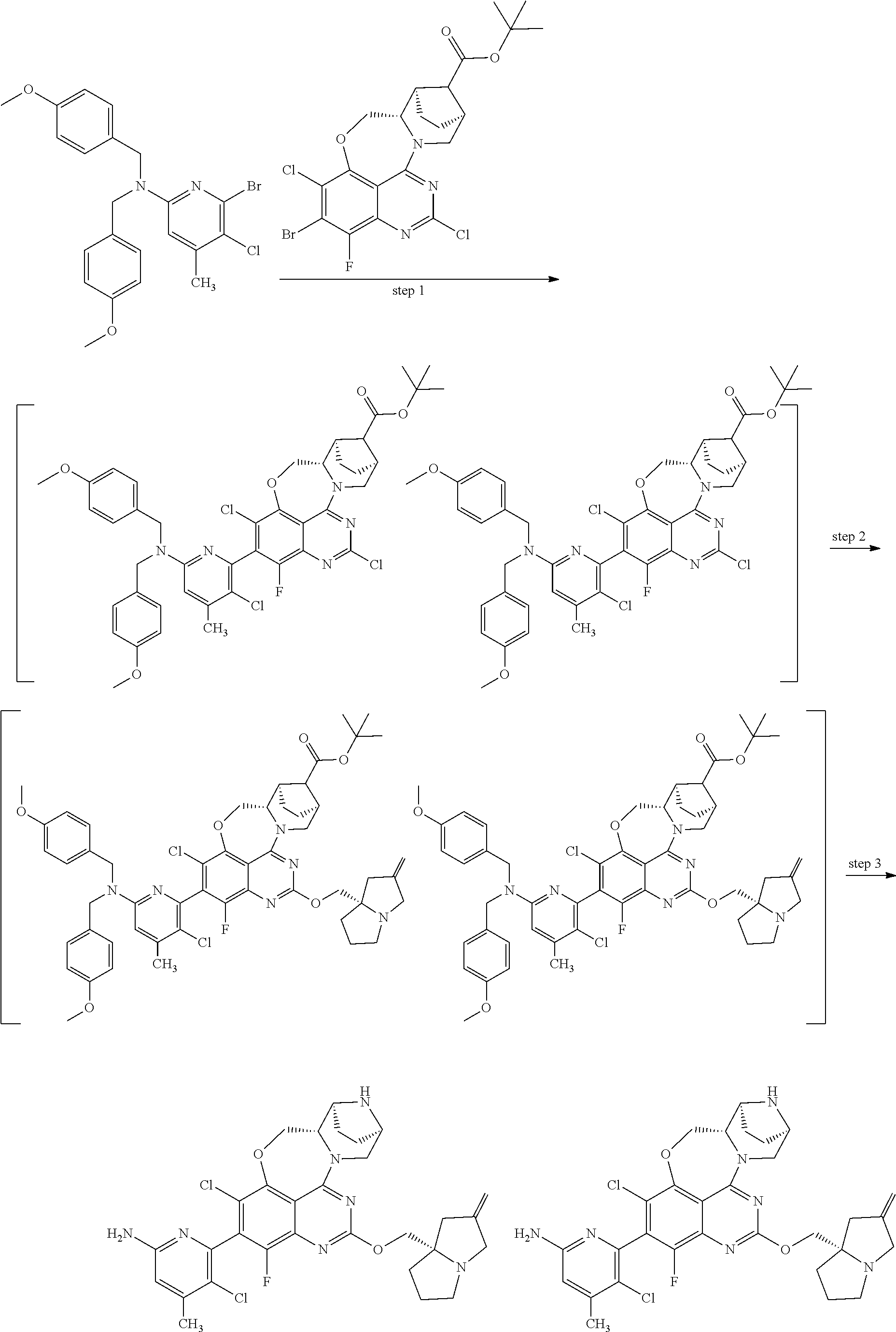

### Step 1: tert-Butyl (5aS,6S,9R)-2-(6-bis(4-methoxybenzyl)amino)-3-chloro-4-methylpyridin-2-yl)-3,13-dichloro-1-fluoro-5a,6,7,8,9,10-hexahydro-5H-6,9-epiminoazepino[2′,1′:3,4][1,4]oxazepino[5,6,7-de]quinazoline-15-carboxylate

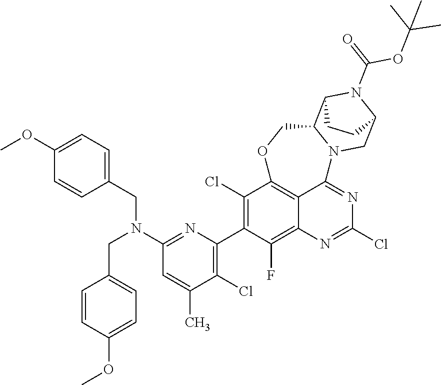 [SMILES: COc1ccc(CN(Cc2ccc(OC)cc2)c2cc(C)c(Cl)c(-c3c(Cl)c4c5c(nc(Cl)nc5c3F)N3C[C@H]5CC[C@@H]([C@H]3CO4)N5C(=O)OC(C)(C)C)n2)cc1]

Under nitrogen, to a solution of tert-butyl (5aS,6S,9R)-2-bromo-3,13-dichloro-1-fluoro-5a,6,7,8,9,10-hexahydro-5H-6,9-epiminoazepino[2′,1′:3,4][1,4]oxazepino[5,6,7-de]quinazoline-15-carboxylate (160 mg, 0.300 mmol, intermediate 3) in THF (1.6 mL) at −78° C. was added a solution of i-PrMgCl·LiCl in THF (1.3 M, 0.26 mL, 0.340 mmol. After 30 min, a solution of ZnCl₂ in 2-methyltetrahydrofuran (2.0 M, 0.2 mL, 0.400 mmol) was added at −78° C. After 5 min, the reaction was warmed to room temperature for 15 min. The above reaction mixture was added into a solution of 6-bromo-5-chloro-N,N-bis(4-methoxybenzyl)-4-methylpyridin-2-amine (110 mg, 0.240 mmol, Intermediate 22) and PdCl₂(PPh₃)₂ (22.0 mg, 0.0300 mmol) in THF (1 mL) under nitrogen. The resulting mixture was stirred at 50° C. overnight under nitrogen. Upon concentration of the reaction, the resulting residue was purified by flash chromatography on silica gel (gradient: 0%-30% ethyl acetate/petroleum ether) to afford 110 mg mixture of diastereomers as a white solid. LCMS (ESI) [M+H]⁺=835. The mixture was separated by Chiral-Prep-HPLC (Column: CHIRALPAK IE-3, 4.6*50 mm 3 um; Mobile Phase: Hex (0.1% DEA): EtOH=70:30). To afford 61 mg of the faster peak and 59 mg of the slower peak as white solids.

### Step 2A: tert-Butyl (5aS,6S,9R)-2-(6-(bis(4-methoxybenzyl)amino)-3-chloro-4-methylpyridin-2-yl)-3-chloro-1-fluoro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate

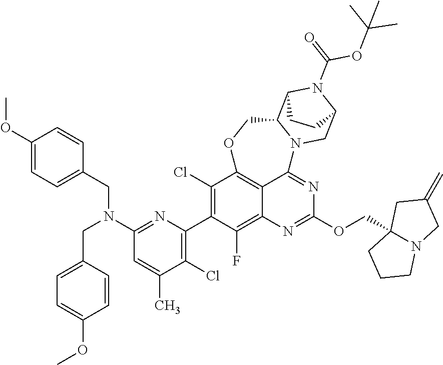 [SMILES: C=C1CN2CCC[C@@]2(COc2nc3c4c(c(Cl)c(-c5nc(N(Cc6ccc(OC)cc6)Cc6ccc(OC)cc6)cc(C)c5Cl)c(F)c4n2)OC[C@@H]2[C@@H]4CC[C@H](CN32)N4C(=O)OC(C)(C)C)C1]

Under nitrogen, a solution of (S)-(2-methylenetetrahydro-1H-pyrrolizin-7a(5H)-yl)methanol (13.8 mg, 0.090 mmol, intermediate 15) in THF (2 mL) was added 60% NaH (12.0 mg, 0.300 mmol) at 0° C. The resulting solution was warmed to room temperature for min. Then tert-butyl (5aS,6S,9R)-2-(6-(bis(4-methoxybenzyl)amino)-3-chloro-4-methylpyridin-2-yl)-3,13-dichloro-1-fluoro-5a,6,7,8,9,10-hexahydro-5H-6,9-epiminoazepino[2′,1′:3,4][1,4]oxazepino[5,6,7-de]quinazoline-15-carboxylate (50.0 mg, 0.0599 mmol, the faster peak of last step) was added at room temperature, and the reaction mixture was heated at 40° C. for 1 hour. The reaction was quenched with saturated aqueous NH₄Cl solution, and the resulting mixture was extracted with (3×). The combined organic layers were dried over anhydrous Na₂SO₄, filtered, and concentrated. The residue was purified by flash chromatography on silica gel (gradient: 0%-8% MeCOH/DCM) to afford 55.9 mg (98% yield) of the title compound as a white solid. LC-MS (ESI, m/z): [M+H]⁺=952.

### Step 2B: tert-Butyl (5aS,6S,9R)-2-(6-(bis(4-methoxybenzyl)amino)-3-chloro-4-methylpyridin-2-yl)-3-chloro-1-fluoro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate

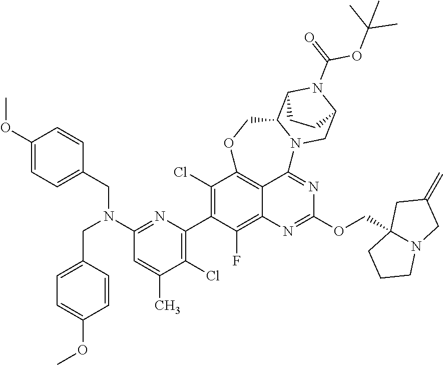 [SMILES: C=C1CN2CCC[C@@]2(COc2nc3c4c(c(Cl)c(-c5nc(N(Cc6ccc(OC)cc6)Cc6ccc(OC)cc6)cc(C)c5Cl)c(F)c4n2)OC[C@@H]2[C@@H]4CC[C@H](CN32)N4C(=O)OC(C)(C)C)C1]

Under nitrogen, a solution of (S)-(2-methylenetetrahydro-1H-pyrrolizin-7a(5H)-yl) methanol (16.2 mg, 0.110 mmol, intermediate 15) in THF (2 mL) was added NaH (14.2 mg, 0.360 mmol) at 0° C. The resulting solution was warmed to temperature. After 30 min, tert-butyl (5aS,6S,9R)-2-(6-(bis(4-methoxybenzyl)amino)-3-chloro-4-methylpyridin-2-yl)-3,13-dichloro-1-fluoro-5a,6,7,8,9,10-hexahydro-5H-6,9-epiminoazepino[2′,1′:3,4][1,4]oxazepino[5,6,7-de]quinazoline-15-carboxylate (59.0 mg, 0.0706 mmol, the slower peak of last step) was added, and the reaction was warmed to 40° C. for 1 hour. The reaction was quenched with saturated aqueous NH₄Cl solution and extracted with EtOAc (3×). The combined organic layers were dried over anhydrous Na₂SO₄, filtered, and concentrated. Purification by flash chromatography on silica gel (gradient: 0%-8% MeCOH/DCM) afforded the title compound as a white solid (60.5 mg, 90% yield). LC-MS (ESI, m/z): [M+H]⁺=952.

### Step 3A: Compound 20A: 5-Chloro-6-((5aS,6S,9R)-3-chloro-1-fluoro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-4-methylpyridin-2-amine

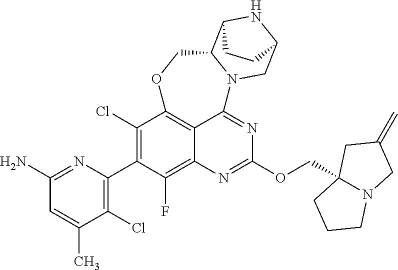 [SMILES: C=C1CN2CCC[C@@]2(COc2nc3c4c(c(Cl)c(-c5nc(N)cc(C)c5Cl)c(F)c4n2)OC[C@@H]2[C@@H]4CC[C@H](CN32)N4)C1]

Under nitrogen, a solution of tert-butyl (5aS,6S,9R)-2-(6-(bis(4-methoxybenzyl)amino)-3-chloro-4-methylpyridin-2-yl)-3-chloro-1-fluoro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate (55.9 mg, 0.0587 mmol) in TFA (2 mL) was stirred at 50° C. for 2 hours. Concentrated under vacuum. The residue was purified by Prep-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 34% B to 43% B in 8 min; Wave Length: 220/254 nm; R_{T} (min): 7.35) to afford 13.2 mg (36% yield) of the title compound as a white solid. LC-MS (ESI, m/z): [M+H]⁺=612. ¹H NMR (300 MHz, DMSO-d₆, ppm) δ56.50 (s, 1H), 6.21 (s, 2H), 4.95-4.80 (m, 3H), 4.59 (dd, J=13.1, 2.7 Hz, 1H), 4.31 (dd, J=13.0, 8.2 Hz, 1H), 4.10-3.88 (m, 3H), 3.55 (d, J=13.5 Hz, 2H), 3.45 (d, J=5.8 Hz, 1H), 3.19 (d, J=14.0 Hz, 1H), 3.09-2.97 (m, 2H), 2.65-2.54 (m, 2H), 2.35 (d, J=15.7 Hz, 1H), 2.30-2.25 (m, 3H), 2.02-1.92 (m, 1H), 1.90-1.78 (m, 2H), 1.76-1.57 (m, 4H), 1.56-1.48 (m, 1H).

### Step 3B: Compound 20B: 5-Chloro-6-((5aS,6S,9R)-3-chloro-1-fluoro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-4-methylpyridin-2-amine

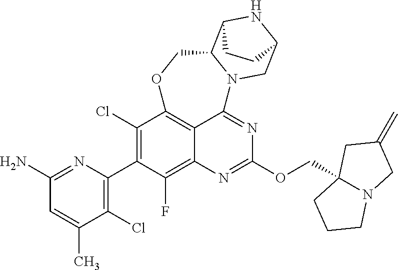 [SMILES: C=C1CN2CCC[C@@]2(COc2nc3c4c(c(Cl)c(-c5nc(N)cc(C)c5Cl)c(F)c4n2)OC[C@@H]2[C@@H]4CC[C@H](CN32)N4)C1]

Under nitrogen, a solution of tert-butyl (5aS,6S,9R)-2-(6-(bis(4-methoxybenzyl)amino)-3-chloro-4-methylpyridin-2-yl)-3-chloro-1-fluoro-13-(((S)-2-methylenetetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate (60.5 mg, 0.0636 mmol) in TFA (2 mL) was stirred at 50° C. for 2 h. The reaction was concentrated under vacuum, and the resulting residue was purified by Prep-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 34% B to 45% B in 8 min; Wave Length: 220/254 nm; R_{T}(min): 7.88;) to afford the title compound as a white solid (13.5 mg, 34% yield). LC-MS (ESI, m/z) [M+H]⁺=612. ¹H NMR (300 MHz, DMSO-d₆, ppm) δ 6.50 (s, 1H), 6.22 (s, 2H), 4.90 (s, 2H), 4.76 (dd, J=12.8, 2.4 Hz, 1H), 4.56 (dd, J=13.1, 2.9 Hz, 1H), 4.39 (dd, J=13.1, 7.3 Hz, 1H), 4.02-3.94 (m, 2H), 3.92 (d, J=10.5 Hz, 1H), 3.55 (d, J=13.4 Hz, 2H), 3.46 (d, J=5.8 Hz, 1H), 3.19 (d, J=14.0 Hz, 1H), 3.11-2.95 (m, 2H), 2.66-2.55 (m, 2H), 2.38 (s, 1H), 2.30-2.20 (m, 3H), 2.02-1.91 (m, 1H), 1.90-1.73 (m, 3H), 1.72-1.60 (m, 3H), 1.60-1.45 (m, 1H).

### Example 21: Compound 21: 6-((2R,5aS,6S,9R)-3-Chloro-1-fluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-4-methyl-5-(trifluoromethyl)pyridin-2-amine

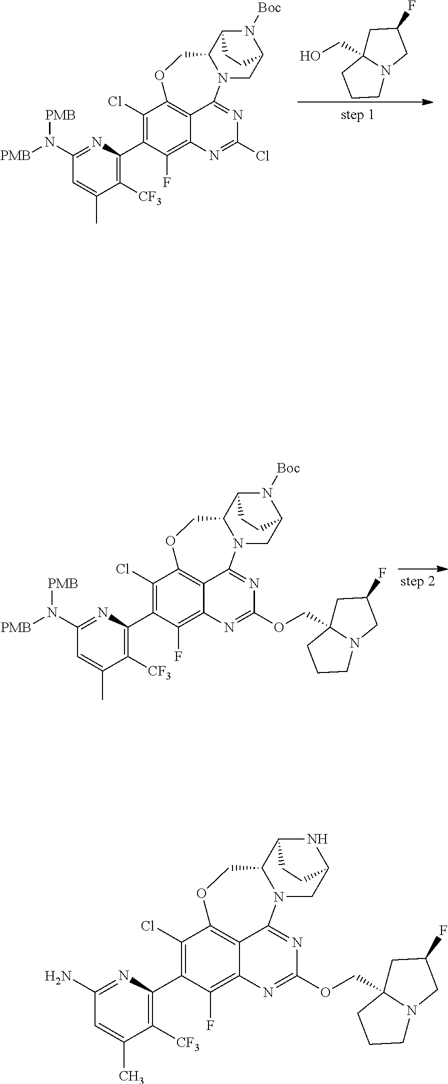

### Step 1: tert-Butyl (2R,5aS,6S,9R)-2-(6-(bis(4-methoxybenzyl)amino)-4-methyl-3-(trifluoromethyl)pyridin-2-yl)-3-chloro-1-fluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate

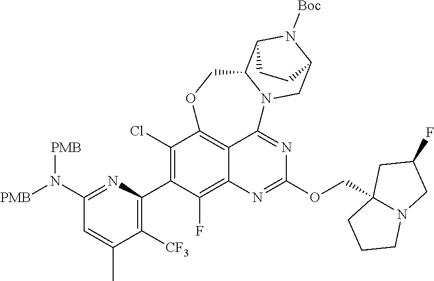

Under N₂, to a solution of ((2R,7aS)-2-fluorotetrahydro-1H-pyrrolizin-7a(5H)-yl)methanol (11.1 mg, 0.07 mmol, intermediate 23) in THF (1.5 mL) was added 60% NaH (3.5 mg, 0.09 mmol) at 0° C. The resulting solution was warmed to room temperature. After min, tert-butyl (2R,5aS,6S,9R)-2-(6-(bis(4-methoxybenzyl)amino)-4-methyl-3-(trifluoromethyl)pyridin-2-yl)-3,13-dichloro-1-fluoro-5a,6,7,8,9,10-hexahydro-5H-6,9-epiminoazepino[2′,1′:3,4][1,4]oxazepino[5,6,7-de]quinazoline-15-carboxylate (50.2 mg, 0.06 mmol, intermediate 5) was added, and the reaction was warmed to 40° C. for 2 h. The reaction was quenched with saturated aqueous NH₄Cl solution and concentrated under vacuum. The residue was purified by flash chromatography on silica gel eluting (gradient: 0-5% MeCOH/DCM) to afford the title compound as a white solid (44.1 mg, 77% yield). LC-MS (ESI, m/z): [M+H]⁺=992

### Step 2: 6-((2R,5aS,6S,9R)-3-Chloro-1-fluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-4-methyl-5-(trifluoromethyl)pyridin-2-amine

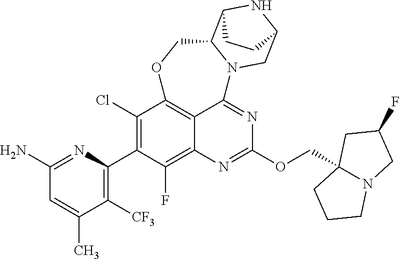

A solution of tert-butyl (2R,5aS,6S,9R)-2-(6-(bis(4-methoxybenzyl)amino)-4-methyl-3-(trifluoromethyl)pyridin-2-yl)-3-chloro-1-fluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline-15-carboxylate (44.1 mg, 0.04 mmol) in TFA (3 mL) was stirred at 50° C. for 4 h. The reaction was concentrated under vacuum. The residue was purified by Prep-HPLC (conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 35% B to 49% B in 8 min, 49% B; Wave Length: 220/254 nm; R_{T1} (min): 7.65) to afford a white solid (14.8 mg, 51.1% yield). LC-MS (ESI, m/z): [M+H]⁺=652. ¹H-NMR (300 MHz, DMSO-d₆): δ 6.84 (s, 2H), 6.47 (s, 1H), 5.28 (d, J=54.2 Hz, 1H), 4.76 (dd, J=12.9, 2.4 Hz, 1H), 4.55 (dd, J=13.2, 2.9 Hz, 1H), 4.36 (dd, J=13.1, 7.2 Hz, 1H), 4.09 (d, J=10.3 Hz, 1H), 3.94 (m, 2H), 3.57 (s, 1H), 3.46 (d, J=5.8 Hz, 1H), 3.18-2.97 (m, 4H), 2.84 (m, 1H), 2.36 (s, 3H), 2.17-2.12 (m, 1H), 2.08-1.96 (m, 2H), 1.91-1.47 (m, 7H)

### Example 22 Compound 22A and Compound 22B: 6-((5aS,6S,9R)-3-chloro-13-(((R)-2,2-difluorotetrahydro-1H-pyrrolizin-7a(5H)-yl)methoxy)-1-fluoro-5a,6,7,8,9,10-hexahydro-5H-6,9-epiminoazepino[2′,1′:3,4][1,4]oxazepino[5,6,7-de]quinazolin-2-yl)-5-(trifluoromethyl)pyridin-2-amine (two single unknown atropisomers)

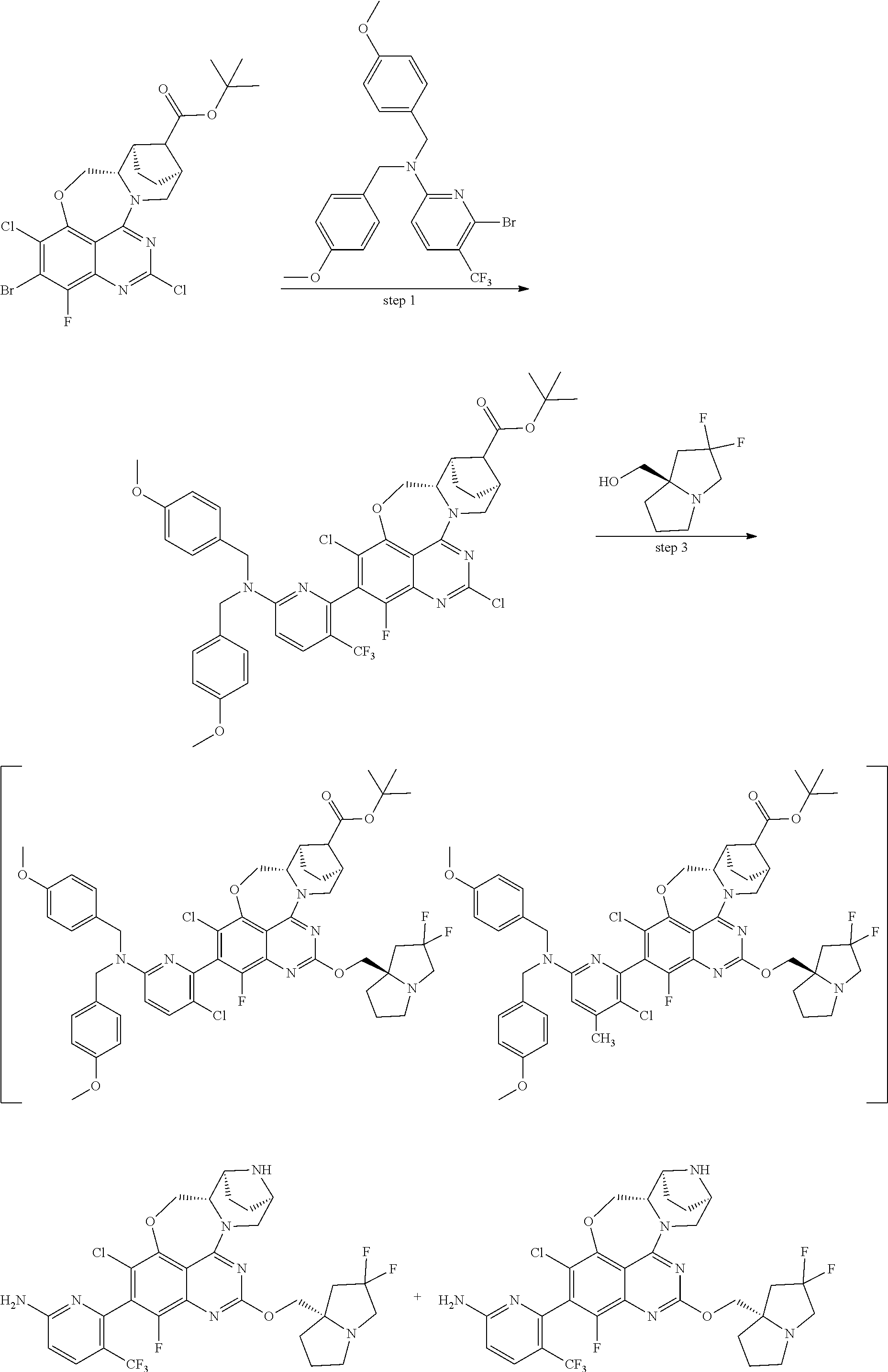

### Step 1: tert-Butyl (5aS,6S,9R)-2-(6-(bis(4-methoxybenzyl)amino)-3-(trifluoromethyl)pyridin-2-yl)-3,13-dichloro-1-fluoro-5a,6,7,8,9,10-hexahydro-5H-6,9-epiminoazepino[2′,1′:3,4][1,4]oxazepino[5,6,7-de]quinazoline-15-carboxylate

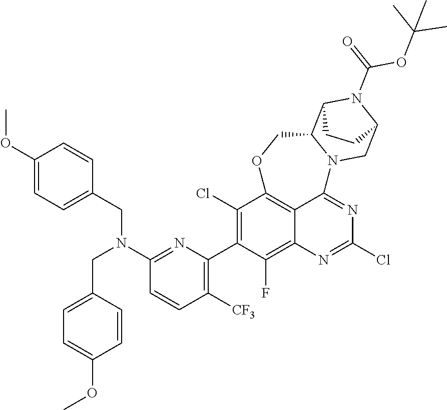 [SMILES: COc1ccc(CN(Cc2ccc(OC)cc2)c2ccc(C(F)(F)F)c(-c3c(Cl)c4c5c(nc(Cl)nc5c3F)N3C[C@H]5CC[C@@H]([C@H]3CO4)N5C(=O)OC(C)(C)C)n2)cc1]

To a solution of tert-butyl (5aS,6S,9R)-2-bromo-3,13-dichloro-1-fluoro-5a,6,7,8,9,10-hexahydro-5H-6,9-epiminoazepino[2′,1′:3,4][1,4]oxazepino[5,6,7-de]quinazoline-15-carboxylate (200 mg, 0.280 mmol, intermediate 3) in tetrahydrofuran (0.76 mL) at −78° C. was added i-PrMgCl·LiCl (1.3 M in THF, 0.3 mL, 0.390 mmol) under nitrogen. After 1 h, ZnCl₂ in 2-methyltetrahydrofuran (2M, 0.2 mL, 0.400 mmol) was added, and the reaction was maintained at −78° C. for 10 min. The reaction was then warmed to room temperature. After 1 h, the reaction was added into a solution of 6-bromo-N,N-bis(4-methoxybenzyl)-5-(trifluoromethyl)pyridin-2-amine (162.7 mg, 0.340 mmol, intermediate 25) and PdCl₂(PPh₃)₂ (10.5 mg, 0.010 mmol) in tetrahydrofuran (0.8 mL) under nitrogen. The solution was stirred at 50° C. overnight. The reaction mixture was diluted with water and extracted with EtOAc (3×). The combined organic layers were dried over anhydrous sodium sulfate, filtered, and concentrated under vacuum. The residue was purified by flash chromatography on silica gel (gradient: 0%-30% EtOAc/petroleum ether) to yield the title compound as a yellow solid (40 mg, 16.6% yield). LC-MS: (ESI, m/z): [M+H]⁺=855.

### Step 2: tert-Butyl (5aS,6S,9R)-2-(6-(bis(4-methoxybenzyl)amino)-3-(trifluoromethyl)pyridin-2-yl)-3-chloro-13-(((R)-2,2-difluorotetrahydro-1H-pyrrolizin-7a(5H)-yl)methoxy)-1-fluoro-5a,6,7,8,9,10-hexahydro-5H-6,9-epiminoazepino[2′,1′:3,4][1,4]oxazepino[5,6,7-de]quinazoline-15-carboxylate (two single known atropisomers)

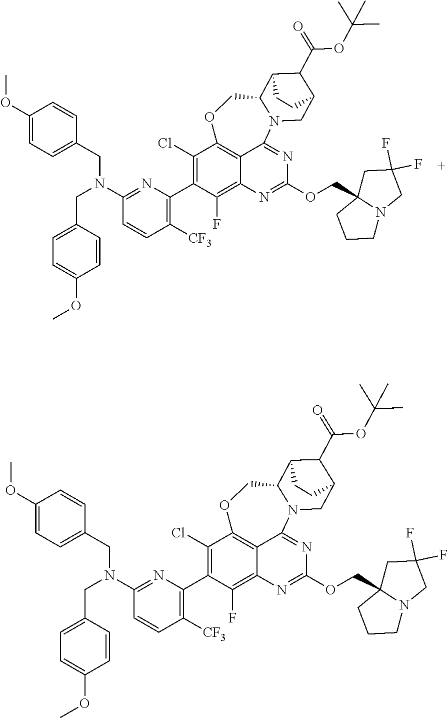 [SMILES: COc1ccc(CN(Cc2ccc(OC)cc2)c2ccc(C(F)(F)F)c(-c3c(Cl)c4c5c(nc(OC[C@]67CCCN6CC(F)(F)C7)nc5c3F)N3C[C@H]5CC[C@H](C5C(=O)OC(C)(C)C)[C@H]3CO4)n2)cc1 ; COc1ccc(CN(Cc2ccc(OC)cc2)c2ccc(C(F)(F)F)c(-c3c(Cl)c4c5c(nc(OC[C@]67CCCN6CC(F)(F)C7)nc5c3F)N3C[C@H]5CC[C@H](C5C(=O)OC(C)(C)C)[C@H]3CO4)n2)cc1]

To an ice-cooled solution of (R)-(2,2-difluorotetrahydro-1H-pyrrolizin-7a(5H)-yl)methanol (29.6 mg, 0.170 mmol, intermediate 17) in tetrahydrofuran (2 mL) was added 60% NaH (29.6 mg, 1.29 mmol) under nitrogen. The reaction was warmed to room temperature. After 30 min, tert-Butyl (5aS,6S,9R)-2-(6-(bis(4-methoxybenzyl)amino)-3-(trifluoromethyl)pyridin-2-yl)-3,13-dichloro-1-fluoro-5a,6,7,8,9,10-hexahydro-5H-6,9-epiminoazepino[2′,1′:3,4][1,4]oxazepino[5,6,7-de]quinazoline-15-carboxylate (110 mg, 0.130 mmol) was added. After 3 h, the reaction was quenched with saturated aqueous NH₄Cl solution and extracted with EtOAc (3×). The combined organic layers were dried over anhydrous sodium sulfate, filtered, and concentrated under vacuum. The residue was purified by flash chromatography on silica gel (gradient: 0%-10% MeCOH/DCM) to yield the title compound as a yellow solid (100 mg mixture of two atropisomers, 78% yield). The mixture was separated by Chiral-HPLC (Column: CHIRAL ART Cellulose-SB, 2*25 cm, 5 μm; Mobile Phase A: MTBE (0.5% 2M NH₃-MeOH)—HPLC, Mobile Phase B: HEX:IPA=13:1; Flow rate: 20 mL/min; Gradient: 70% B to 70% B in 15 min; Wave Length: 220/254 nm; RT1 (min): 12.221; RT2 (min): 13.85; Sample Solvent: EtOH:DCM=1:1—HPLC; Injection Volume: 0.7 mL; Number Of Runs: 12.) to yield 50.0 mg of the faster peak and 45.0 mg of the slower peak as white solids. LC-MS: (ESI, m/z): [M+H]⁺=996.

### Step 3: 6-((5aS,6S,9R)-3-chloro-13-(((R)-2,2-difluorotetrahydro-1H-pyrrolizin-7a(5H)-yl)methoxy)-1-fluoro-5a,6,7,8,9,10-hexahydro-5H-6,9-epiminoazepino[2′,1′:3,4][1,4]oxazepino[5,6,7-de]quinazolin-2-yl)-5-(trifluoromethyl)pyridin-2-amine (two single unknown atropisomers)

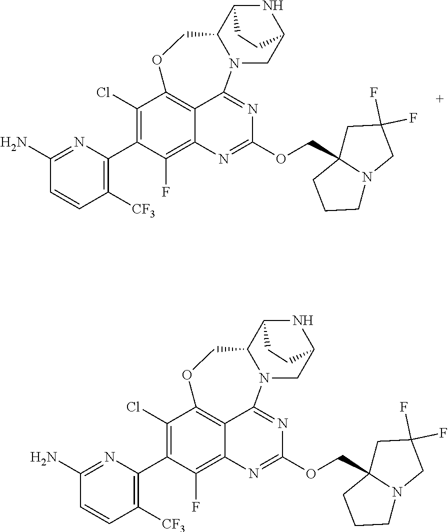 [SMILES: Nc1ccc(C(F)(F)F)c(-c2c(Cl)c3c4c(nc(OC[C@]56CCCN5CC(F)(F)C6)nc4c2F)N2C[C@H]4CC[C@H](N4)[C@H]2CO3)n1 ; Nc1ccc(C(F)(F)F)c(-c2c(Cl)c3c4c(nc(OC[C@]56CCCN5CC(F)(F)C6)nc4c2F)N2C[C@H]4CC[C@H](N4)[C@H]2CO3)n1]

A solution of tert-Butyl (5aS,6S,9R)-2-(6-(bis(4-methoxybenzyl)amino)-3-(trifluoromethyl)pyridin-2-yl)-3-chloro-13-(((R)-2,2-difluorotetrahydro-1H-pyrrolizin-7a(5H)-yl)methoxy)-1-fluoro-5a,6,7,8,9,10-hexahydro-5H-6,9-epiminoazepino[2′,1′:3,4][1,4]oxazepino[5,6,7-de]quinazoline-15-carboxylate (50.0 mg, 0.050 mmol, the faster peak of last step) in TFA (2 mL) was stirred at 50° C. for 3 h. The reaction mixture was concentrated under vacuum, and the resulting residue was purified by Prep-HPLC (Column: XBridge Prep OBD C18 Column, 30×150 mm 5 μm; Mobile Phase A: Water (10 mmol/L NH₄HCO₃), Mobile Phase B: ACN; Flow rate: 60 mL/min; Gradient: 40 B to 50 B in 8 min, 254/220 nm; RT:7.32.) to yield 6.0 mg (18% yield) of Compound 22B as a white solid. LC-MS: (ESI, m/z): [M+H]⁺=656.15. ¹H NMR (400 MHz, DMSO-d₆, ppm) δ 7.80 (d, J=8.8 Hz, 1H), 6.96 (s, 2H), 6.61 (d, J=8.8 Hz, 1H), 4.86 (dd, J=13.2, 2.4 Hz, 1H), 4.59 (dd, J=13.2, 2.8 Hz, 1H), 4.30 (dd, J=12.8, 8.4 Hz, 1H), 4.15-4.01 (m, 3H), 3.61-3.52 (m, 1H), 3.49-3.41 (m, 1H), 3.39-3.35 (m, 1H), 3.17-3.01 (m, 3H), 2.81-2.67 (m, 2H), 2.49-2.33 (m, 2H), 2.09-1.99 (m, 1H), 1.98-1.86 (m, 1H), 1.83-1.73 (m, 2H), 1.72-1.61 (m, 3H), 1.60-1.49 (m, 1H).

Analogous to method described in Compound 22B, Compound 22A was prepared from tert-butyl(5aS,6S,9R)-2-(6-(bis(4-methoxybenzyl)amino)-3-(trifluoromethyl) pyridin-2-yl)-3-chloro-13-(((R)-2,2-difluorotetrahydro-1H-pyrrolizin-7a(5H)-yl)methoxy)-1-fluoro-5a,6,7,8,9,10-hexahydro-5H-6,9-epiminoazepino[2′,1′:3,4][1,4]oxazepino[5,6,7-de]quinazoline-15-carboxylate (45 mg, the slower peak of last step) and purified by Prep-HPLC (Column: XBridge Prep OBD C18 Column, 30×150 mm 5 μm; Mobile Phase A: Water (10 mmol/L NH₄HCO₃), Mobile Phase B: ACN; Flow rate: 60 mL/min; Gradient: 45 B to 55 B in 8 min, 254/220 nm; RT:6.52.) to yield 9.0 mg (30% yield) as a white solid. LC-MS: (ESI, m/z): [M+H]⁺=656.15. ¹H NMR (400 MHz, DMSO-d₆, ppm) δ 7.80 (d, J=8.8 Hz, 1H), 6.96 (s, 2H), 6.61 (d, J=8.8 Hz, 1H), 4.77 (dd, J=12.8, 2.0 Hz, 1H), 4.54 (d, J=2.4 Hz, 1H), 4.47-4.29 (m, 1H), 4.12 (d, J=10.8 Hz, 1H), 4.05-3.97 (m, 2H), 3.63-3.53 (m, 1H), 3.52-3.44 (m, 1H), 3.31-3.27 (m, 1H), 3.17-3.01 (m, 3H), 2.91 (s, 1H), 2.78-2.67 (m, 1H), 2.47-2.31 (m, 2H), 2.09-1.97 (m, 1H), 1.96-1.83 (m, 1H), 1.82-1.72 (m, 3H), 1.71-1.49 (m, 3H).

### Example 23: Compound 23 6-((5S,5aS,6S,9R)-3-Chloro-1-fluoro-13-(((2R,7aS)-2-fluorotetrahydro-1H-pyrrolizin-7a(5H)-yl)methoxy)-5-methyl-5a,6,7,8,9,10-hexahydro-5H-6,9-epiminoazepino[2′,1′:3,4][1,4]oxazepino[5,6,7-de]quinazolin-2-yl)-4-methyl-5-(trifluoromethyl)pyridin-2-amine

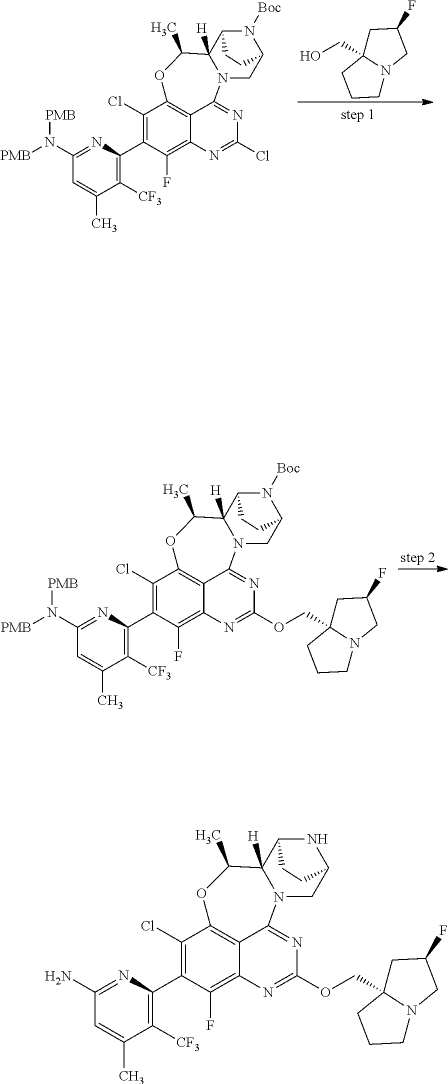

### Step 1: tert-Butyl (2R,5S,5aS,6S,9R)-2-(6-(bis(4-methoxybenzyl)amino)-4-methyl-3-(trifluoromethyl)pyridin-2-yl)-3-chloro-1-fluoro-13-(((2R,7aS)-2-fluorotetrahydro-1H-pyrrolizin-7a(5H)-yl)methoxy)-5-methyl-5a,6,7,8,9,10-hexahydro-5H-6,9-epiminoazepino[2′,1′:3,4][1,4]oxazepino[5,6,7-de]quinazoline-15-carboxylate

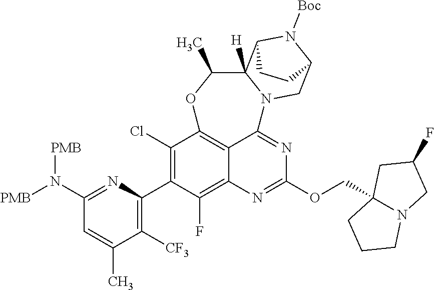

Under nitrogen, to a solution of ((2R,7aS)-2-fluorotetrahydro-1H-pyrrolizin-7a(5H)-yl)methanol (16.4 mg, 0.100 mmol, Intermediate 16) in tetrahydrofuran (2 mL) was added NaH (8.20 mg, 0.200 mmol) at 0° C. The resulting solution was stirred for 30 min at 0° C. Then tert-butyl (2R,5S,5aS,6S,9R)-2-(6-(bis(4-methoxybenzyl)amino)-4-methyl-3-(trifluoromethyl)pyridin-2-yl)-3,13-dichloro-1-fluoro-5-methyl-5a,6,7,8,9,10-hexahydro-5H-6,9-epiminoazepino[2′,1′:3,4][1,4]oxazepino[5,6,7-de]quinazoline-15-carboxylate (70.0 mg, 0.0800 mmol, the faster peak of intermediate 30) was added at 0° C. and stirred for 1 h at room temperature. The reaction was quenched with water and concentrated under vacuum. The residue was purified by flash chromatography on silica gel (0-10% MeCOH in DCM) to yield the title compound 62 mg (77.8% yield) as a white solid. LC-MS: (ESI, m/z): [M+H]⁺=1006.

### Step 2: 6-((2R,5S,5aS,6S,9R)-3-chloro-1-fluoro-13-(((2R,7aS)-2-fluorotetrahydro-1H-pyrrolizin-7a(5H)-yl)methoxy)-5-methyl-5a,6,7,8,9,10-hexahydro-5H-6,9-epiminoazepino[2′,1′:3,4][1,4]oxazepino[5,6,7-de]quinazolin-2-yl)-4-methyl-5-(trifluoromethyl)pyridin-2-amine

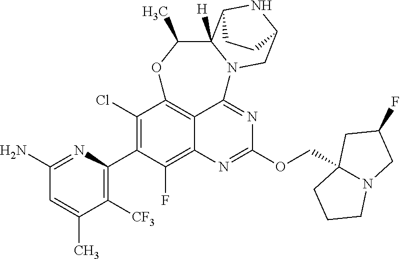

A solution of tert-butyl (5S,5aS,6S,9R)-2-(6-(bis(4-methoxybenzyl)amino)-4-methyl-3-(trifluoromethyl)pyridin-2-yl)-3-chloro-1-fluoro-13-(((2R,7aS)-2-fluorotetrahydro-1H-pyrrolizin-7a(5H)-yl)methoxy)-5-methyl-5a,6,7,8,9,10-hexahydro-5H-6,9-epiminoazepino[2′,1′:3,4][1,4]oxazepino[5,6,7-de]quinazoline-15-carboxylate (60.0 mg, 0.0600 mmol) in TFA (2 mL) was stirred for 4 h at 50° C. Solvent was evaporated under vacuum. The residue was purified by pre-packed C18 column (solvent gradient: 0-100% ACN in water (0.05% NH₄HCO₃)) to yield the title compound (23.5 mg, 59.2% yield) as a white solid. LC-MS: (ESI, m/z): [M+H]⁺=666. ¹H NMR (400 MHz, DMSO-d₆) δ 6.84 (s, 2H), 6.46 (s, 1H), 5.27 (d, J=54 Hz, 1H), 5.15-5.12 (m, 1H), 4.34-4.30 (m, 1H), 4.07 (d, J=10 Hz, 1H), 3.96 (t, J=9 Hz, 2H), 3.55 (s, 1H), 3.38 (d, J=6 Hz, 1H), 3.33-3.06 (m, 2H), 3.02-3.00 (m, 2H), 2.849-2.79 (m, 1H), 2.36 (s, 3H), 2.13-2.11 (m, 1H), 2.04-1.98 (m, 2H), 1.87-1.72 (m, 4H), 1.62-1.58 (m, 2H), 1.53-1.47 (m, 4H).

### Example 24: Compound 24: 6-((2S,5S,5aS,6S,9R)-3-Chloro-1-fluoro-13-(((2R,7aS)-2-fluorotetrahydro-1H-pyrrolizin-7a(5H)-yl)methoxy)-5-methyl-5a,6,7,8,9,10-hexahydro-5H-6,9-epiminoazepino[2′,1′:3,4][1,4]oxazepino[5,6,7-de]quinazolin-2-yl)-4-methyl-5-(trifluoromethyl)pyridin-2-amine

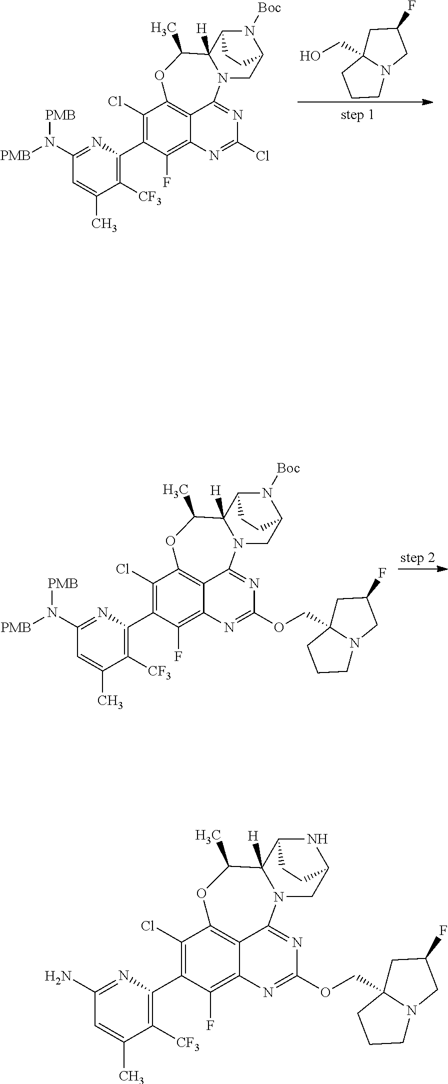

### Step 1: tert-Butyl (2S,5S,5aS,6S,9R)-2-(6-(bis(4-methoxybenzyl)amino)-4-methyl-3-(trifluoro methyl)pyridin-2-yl)-3-chloro-1-fluoro-13-(((2R,7aS)-2-fluorotetrahydro-1H-pyrrolizin-7a(5H)-yl)methoxy)-5-methyl-5a,6,7,8,9,10-hexahydro-5H-6,9-epiminoazepino[2′,1′:3,4][1,4]oxazepino[5,6,7-de]quinazoline-15-carboxylate

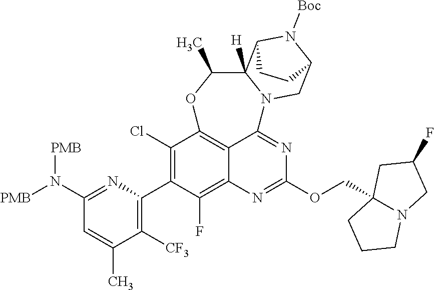

Under nitrogen, to a solution of ((2R,7aS)-2-fluorotetrahydro-1H-pyrrolizin-7a(5H)-yl)methanol (27.1 mg, 0.170 mmol, Intermediate 16) in tetrahydrofuran (2 mL) was added NaH (13.6 mg, 0.340 mmol) at 0° C. The result solution was stirred for 30 min at 0° C. Then tert-butyl (2R,5S,5aS,6S,9R)-2-(6-(bis(4-methoxybenzyl)amino)-4-methyl-3-(trifluoromethyl)pyridin-2-yl)-3,13-dichloro-1-fluoro-5-methyl-5a,6,7,8,9,10-hexahydro-5H-6,9-epiminoazepino[2′,1′:3,4][1,4]oxazepino[5,6,7-de]quinazoline-15-carboxylate (100 mg, 0.110 mmol, the slower peak of intermediate 30) was added at 0° C. and stirred for 1 h at room temperature. The reaction was quenched with water and evaporated under vacuum. The residue was purified by flash chromatography on silica gel (0-10% MeCOH in DCM) to yield the title compound (90 mg, 79% yield) as a yellow solid. LC-MS: (ESI, m/z): [M+H]⁺=1006.

### Step 2: 6-((2S,5S,5aS,6S,9R)-3-Chloro-1-fluoro-13-(((2R,7aS)-2-fluorotetrahydro-1H-pyrrolizin-7a(5H)-yl)methoxy)-5-methyl-5a,6,7,8,9,10-hexahydro-5H-6,9-epiminoazepino[2′,1′:3,4][1,4]oxazepino[5,6,7-de]quinazolin-2-yl)-4-methyl-5-(trifluoromethyl)pyridin-2-amine

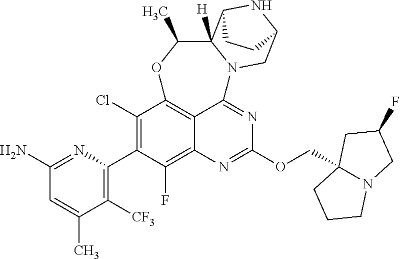

A solution of tert-butyl (5S,5aS,6S,9R)-2-(6-(bis(4-methoxybenzyl)amino)-4-methyl-3-(trifluoromethyl)pyridin-2-yl)-3-chloro-1-fluoro-13-(((2R,7aS)-2-fluorotetrahydro-1H-pyrrolizin-7a(5H)-yl)methoxy)-5-methyl-5a,6,7,8,9,10-hexahydro-5H-6,9-epiminoazepino[2′,1′:3,4][1,4]oxazepino[5,6,7-de]quinazoline-15-carboxylate (90.0 mg, 0.0900 mmol) in TFA (2 mL) was stirred for 4 h at 50° C. Solvent was evaporated under vacuum. The residue was purified by pre-packed 018 column (solvent gradient: 0-100% ACN in water (0.05% NH₄HCO₃)) to yield the title compound (38.3 mg, 64.3% yield) as a white solid. LC-MS: (ESI, m/z): [M+H]⁺=666. ¹H NMR (400 MHz, DMSO-d₆) δ 6.83 (s, 2H), 6.47 (s, 1H), 5.27 (d, J=54.4 Hz, 1H), 5.16-5.13 (m, 1H), 4.32-4.28 (m, 1H), 4.05-3.94 (m, 3H), 3.55 (d, J=4.4 Hz, 1H), 3.39-3.33 (m, 1H), 3.09-3.07 (m, 2H), 3.02-3.00 (m, 2H), 2.85-2.79 (m, 2H), 2.35 (s, 3H), 2.14-2.12 (m, 1H), 2.08-2.00 (m, 2H), 1.92-1.73 (m, 4H), 1.60-1.46 (m, 6H).

Each compound in Table below was prepared following a similar experimental procedure (using appropriately substituted reagents) as described for Example 23.

| Cmpd | |
|---|---|
| No | Structure |
| 23 | 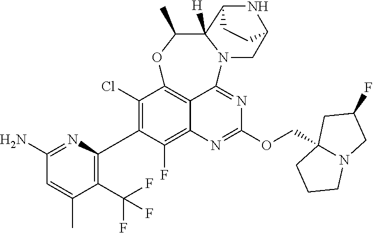 |
| 24 | 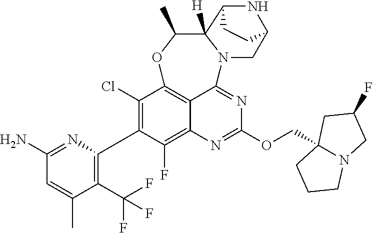 |
| 25 | 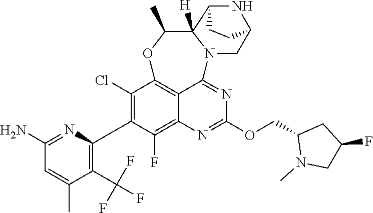 |
| 26 | 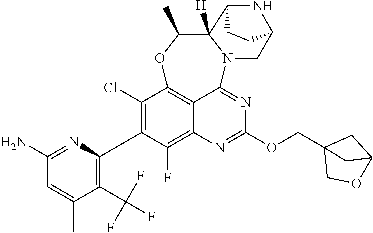 |
| 27 | 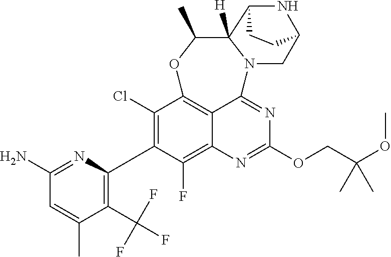 |
| 28 | 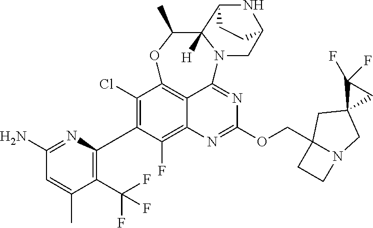 |
| 29 | 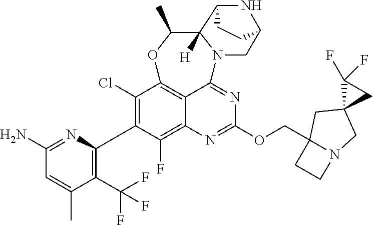 |
| 30A | 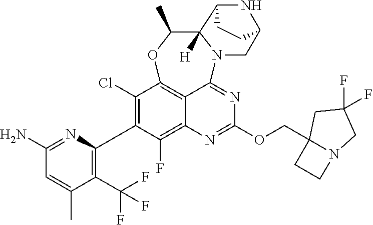 |
| 30B | 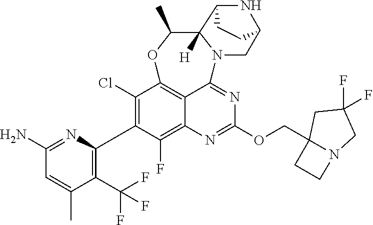 |
| 31 | 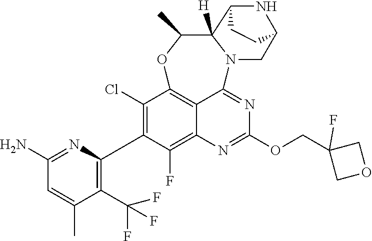 |
| 32 | 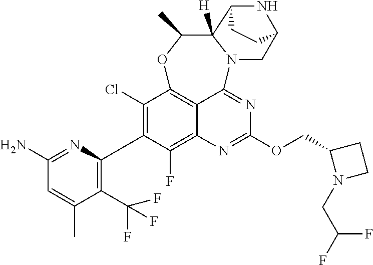 |
| 33 | 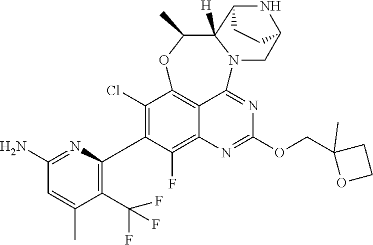 |
| 34 | 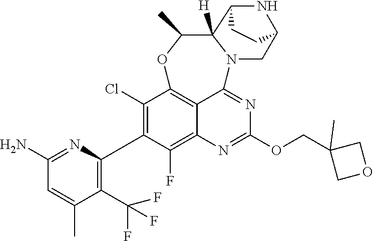 |
| 35 | 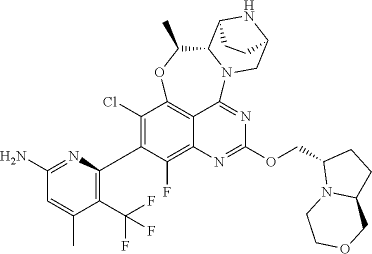 |
| 36 | 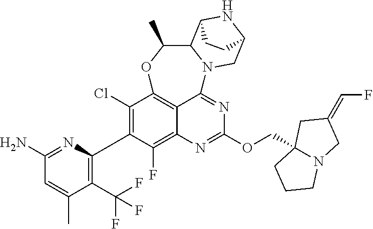 |
| 37 | 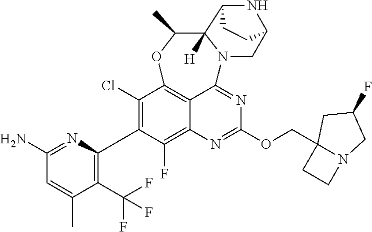 |

### Example 38: Compound 38A and 38B3: 6-((2R,5S,5aS,6S,9R)-3-Chloro-13-((3,3-difluoro-1-azabicyclo[3.2.0]heptan-5-yl)methoxy)-1-fluoro-5-methyl-5a,6,7,8,9,10-hexahydro-5H-6,9-epiminoazepino[2′,1′:3,4][1,4]oxazepino[5,6,7-de]quinazolin-2-yl)-5-(trifluoromethyl) pyridin-2-amine (two unknown single isomers)

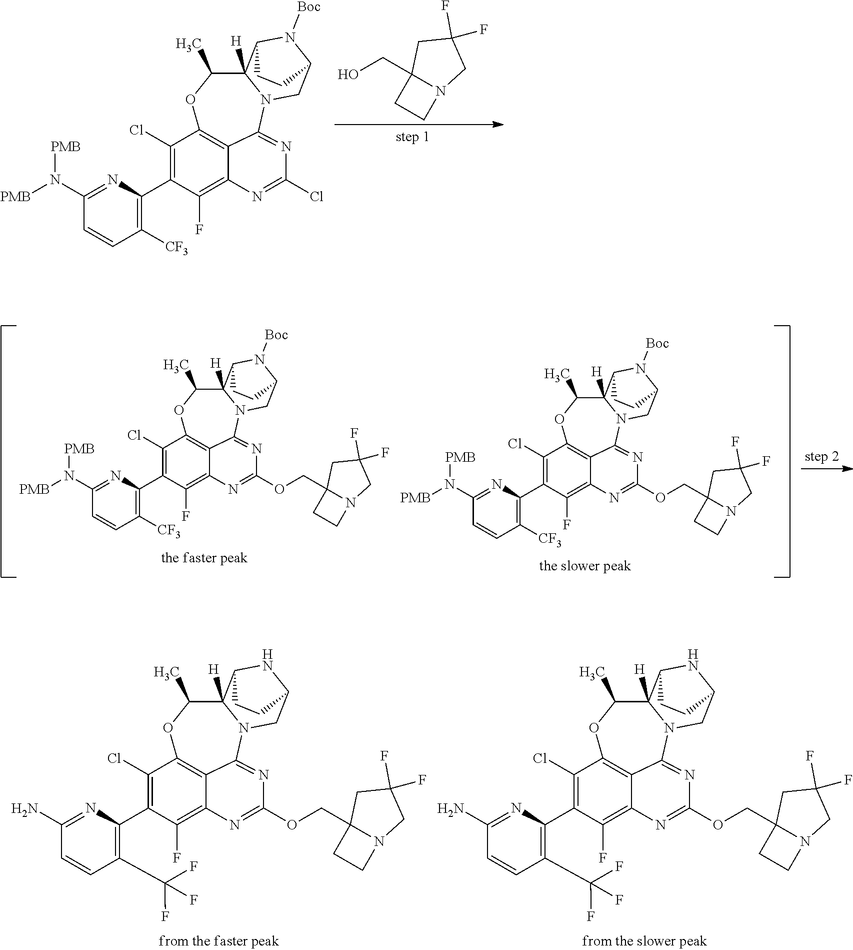

### Step 1: tert-Butyl (2R,5S,5aS,6S,9R)-2-(6-(bis(4-methoxybenzyl)amino)-3-(trifluoromethyl) pyridin-2-yl)-3-chloro-13-((3,3-difluoro-1-azabicyclo[3.2.0]heptan-5-yl)methoxy)-1-fluoro-5-methyl-5a,6,7,8,9,10-hexahydro-5H-6,9-epiminoazepino[2′,1′:3,4][1,4]oxazepino[5,6,7-de]quinazoline-15-carboxylate

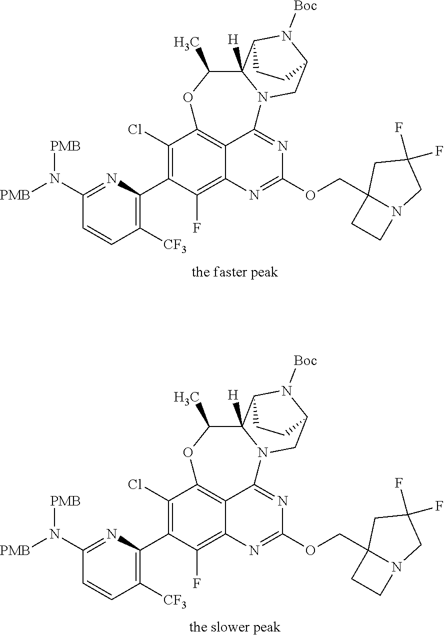

Under nitrogen, to a solution of (3,3-difluoro-1-azabicyclo[3.2.0]heptan-5-yl)methanol (31.5 mg, 0.190 mmol, Intermediate 35) in tetrahydrofuran (2 mL) was added NaH (16.1 mg, 0.400 mmol, 60% suspend in oil) at 0° C. The resulting solution was stirred for 30 min at 0° C. Then tert-butyl (2R,5S,5aS,6S,9R)-2-(6-(bis(4-methoxybenzyl)amino)-3-(trifluoromethyl)pyridin-2-yl)-3,13-dichloro-4-fluoro-5-methyl-5a,6,7,8,9,10-hexahydro-5H-6,9-epiminoazepino[2′,1′:3,4][1,4]oxazepino[5,6,7-de]quinazoline-15-carboxylate (140 mg, 0.160 mmol, the slower peak of Intermediate 31) was added and the reaction was stirred for 2 h at 40° C. The reaction was quenched with water and concentrated under vacuum. The residue was purified by flash chromatography on silica gel (0-10% MeOH in DCM) to yield 120 mg of title compound (mixture of 2 isomers) as a yellow solid. The mixture was separated by PREP_CHIRAL_HPLC (Column: CHIRAL ART Cellulose-SB, 2*25 cm, 5 μm; Mobile Phase A: Hex (0.5% 2M NH₃-MeOH)—HPLC, Mobile Phase B: EtOH—HPLC; Flow rate: 20 mL/min; Gradient: 15% B to 15% B in 21 min; Wave Length: 220/254 nm; RT1 (min): 14.721; RT2 (min): 17.854; Sample Solvent: EtOH—HPLC; Injection Volume: 0.5 mL; Number Of Runs: 8) to yield the faster peak (58 mg, 36.2% yield) and the slower peak (56 mg, 34.9% yield) as yellow solids. LC-MS: (ESI, m/z): [M+H]⁺=996.

### Step 2: 6-((2R,5S,5aS,6S,9R)-3-Chloro-13-((3,3-difluoro-1-azabicyclo[3.2.0]heptan-5-yl)methoxy)-1-fluoro-5-methyl-5a,6,7,8,9,10-hexahydro-5H-6,9-epiminoazepino[2′,1′:3,4][1,4]oxazepino[5,6,7-de]quinazolin-2-yl)-5-(trifluoromethyl)pyridin-2-amine (two single unknown isomers)

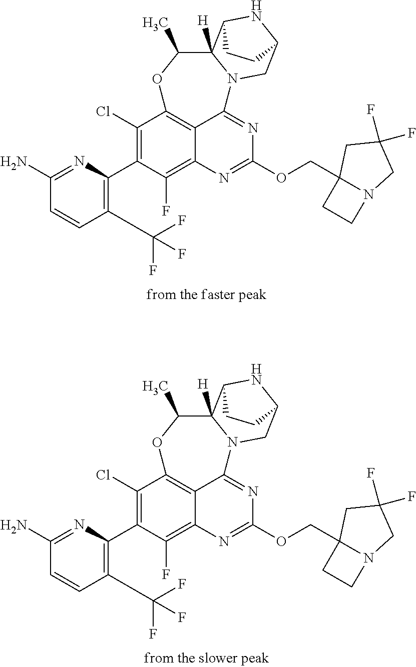

A solution of tert-butyl (2R,5S,5aS,6S,9R)-2-(6-(bis(4-methoxybenzyl)amino)-3-(trifluoromethyl)pyridin-2-yl)-3-chloro-13-((3,3-difluoro-1-azabicyclo[3.2.0]heptan-5-yl)methoxy)-1-fluoro-5-methyl-5a,6,7,8,9,10-hexahydro-5H-6,9-epiminoazepino[2′,1′:3,4][1,4]oxazepino[5,6,7-de]quinazoline-15-carboxylate (58.0 mg, 0.0600 mmol, the faster peak of step 1) in TFA (2 mL) was stirred for 3 h at 50° C. Solvent was evaporated under vacuum. The residue was purified by pre-packed C18 column (0-100% ACN in water (0.05% NH₄HCO₃)) to yield Compound 38 (25 mg, 65.5% yield) as a white solid. LC-MS: (ESI, m/z): [M+H]⁺=656. ¹H NMR (400 MHz, DMSO-d₆) δ 7.80 (d, J=8.9 Hz, 1H), 6.97 (s, 2H), 6.61 (d, J=8.8 Hz, 1H), 5.18-5.14 (m, 1H), 4.41-4.23 (m, 3H), 3.99 (d, J=9.4 Hz, 1H), 3.55-3.51 (m, 2H), 3.40 (d, J=6.2 Hz, 1H), 3.23-3.20 (m, 1H), 3.13 (d, J=4.3 Hz, 1H), 3.09-2.98 (m, 2H), 2.68-2.58 (m, 1H), 2.49-2.26 (m, 3H), 1.95-1.84 (m, 1H), 1.66-1.51 (m, 2H), 1.48 (d, J=6.3 Hz, 4H).

A solution of tert-Butyl (2R,5S,5aS,6S,9R)-2-(6-(bis(4-methoxybenzyl)amino)-3-(trifluoromethyl)pyridin-2-yl)-3-chloro-13-((3,3-difluoro-1-azabicyclo[3.2.0]heptan-5-yl)methoxy)-1-fluoro-5-methyl-5a,6,7,8,9,10-hexahydro-5H-6,9-epiminoazepino[2′,1′:3,4][1,4]oxazepino[5,6,7-de]quinazoline-15-carboxylate (56.0 mg, 0.0600 mmol, the slower peak of step 1) in TFA (2 mL) was stirred for 3 h at 50° C. Solvent was evaporated under vacuum. The residue was purified by pre-packed 018 column (0-100% ACN in water (0.05% NH₄HCO₃)) to yield Compound 38 (20.9 mg, 56.7% yield) as a white solid. LC-MS: (ESI, m/z): [M+H]⁺=656. ¹H NMR (400 MHz, DMSO-d₆) δ 7.80 (d, J=9.0 Hz, 1H), 6.97 (s, 2H), 6.61 (d, J=8.8 Hz, 1H), 5.18-5.14 (m, 1H), 4.41-4.30 (m, 2H), 4.27 (d, J=11.0 Hz, 1H), 3.99 (d, J=9.4 Hz, 1H), 3.56-3.52 (m, 2H), 3.40 (d, J=6.1 Hz, 1H), 3.22-3.08 (m, 1H), 3.13 (d, J=4.7 Hz, 1H), 3.10-2.98 (m, 2H), 2.87 (s, 1H), 2.70-2.56 (m, 1H), 2.50-2.25 (m, 3H), 1.93-1.84 (m, 1H), 1.65-1.60 (m, 2H), 1.48 (d, J=6.3 Hz, 4H).

Each compound in Table below was prepared following a similar experimental procedure (using appropriately substituted reagents) as described Example 38.

| Cmpd | |
|---|---|
| No. | Structure |
| 38A | 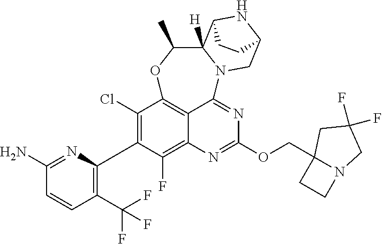 |
| 38B | 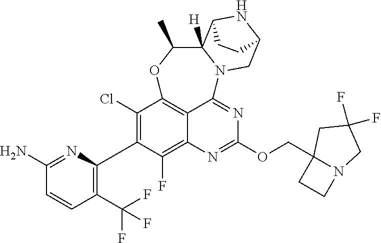 |
| 39 | 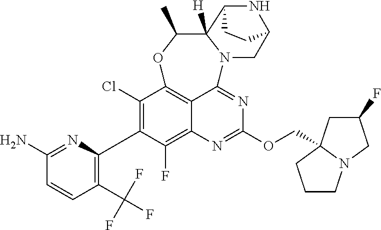 |
| 40 | 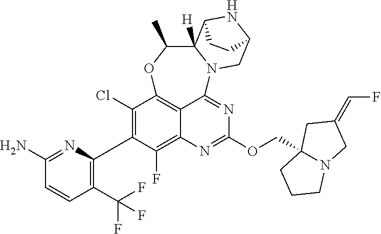 |
| 41A | 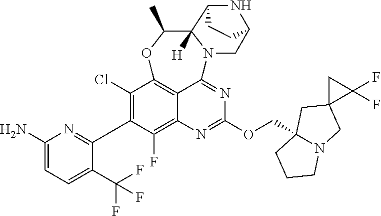 |
| 41B | 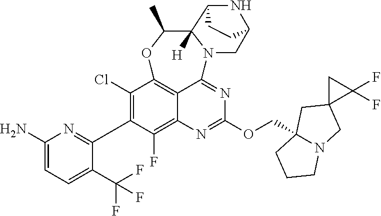 |

### Example 42: Compound 42A and 42B: 6-((5S,5aS,6S,9R)-3-Chloro-1-fluoro-5-methyl-5a,6,7,8,9,10-hexahydro-5H-6,9-epiminoazepino[2′,1′-3,4][1,4]oxazepino[5,6,7-de]quinazolin-2-yl)-4-methyl-5-(trifluoromethyl)pyridin-2-amine

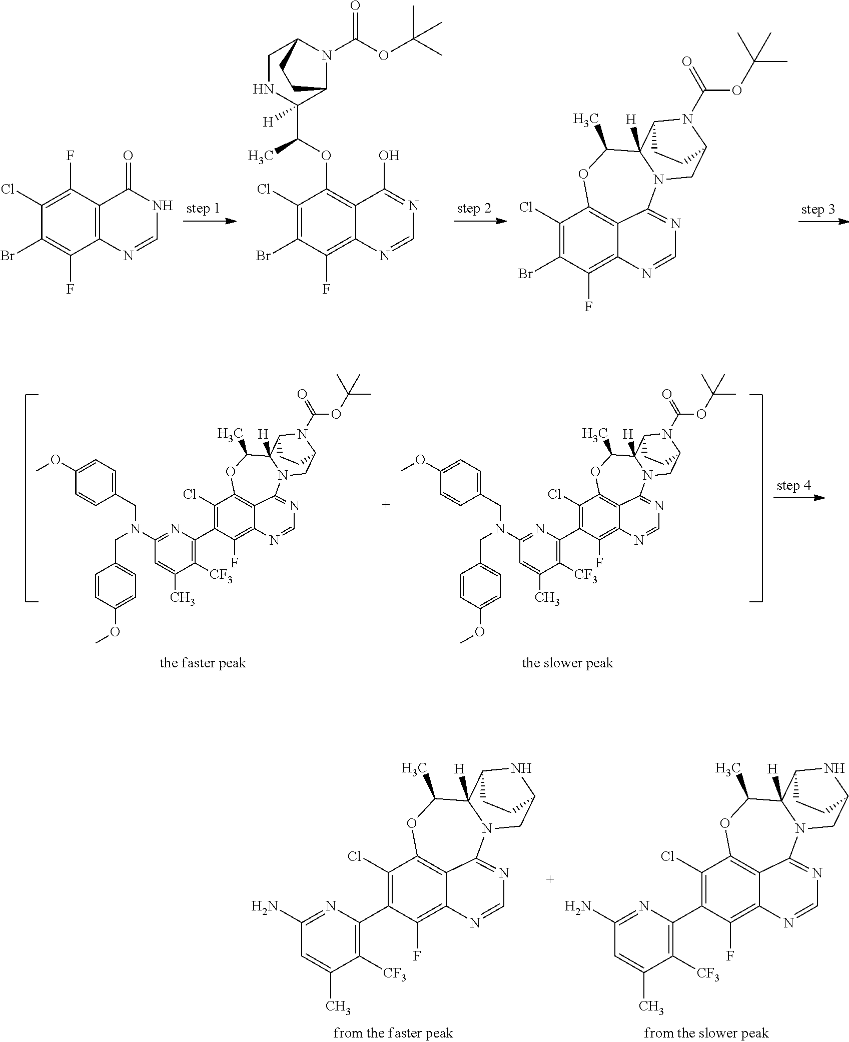

### Step 1: tert-Butyl (1 S,2S,5R)-2-((S)-1-((7-bromo-6-chloro-8-fluoro-4-hydroxyquinazolin-5-yl)oxy)ethyl)-3,8-diazabicyclo[3.2.1]octane-8-carboxylate

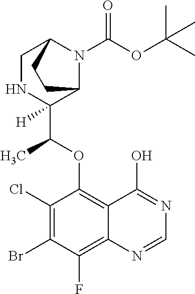 [SMILES: C[C@H](Oc1c(Cl)c(Br)c(F)c2ncnc(O)c12)[C@H]1NC[C@H]2CC[C@@H]1N2C(=O)OC(C)(C)C]

Under nitrogen, to a solution of tert-butyl (1R,2S,5S)-2-((S)-1-hydroxyethyl)-3,8-diazabicyclo[3.2.1]octane-8-carboxylate (500 mg, 1.95 mmol, Intermediate 29) in THF (15 mL) was added NaH (586 mg, 14.7 mmol, 60% in mineral oil) at 0° C. The resulting solution was stirred at room temperature for 30 minutes. Then 7-bromo-6-chloro-5,8-difluoroquinazolin-4 (3H)-one (1.44 g, 4.86 mmol, Intermediate 26) was added to the reaction solution at 0° C. The reaction system was stirred at room temperature for 1 h. The solution was quenched with NH₄Cl aqueous and concentrated under vacuum. The residue was purified by flash chromatography on silica gel (gradient: 0%-8% MeCOH/DCM) to yield 481 mg (18.6% yield) of the title compound as a yellow solid. LC-MS: (ESI, m/z): [M+H]⁺=533.

### Step 2: tert-Butyl (5S,5aS,6S,9R)-2-bromo-3-chloro-1-fluoro-5-methyl-5a,6,7,8,9,10-hexahydro-5H-6,9-epiminoazepino[2′,1′:3,4][1,4]oxazepino[5,6,7-de]quinazoline-15-carboxylate

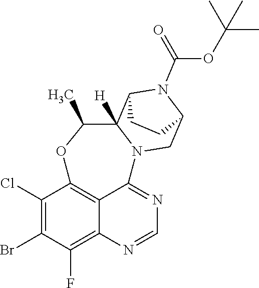 [SMILES: C[C@@H]1Oc2c(Cl)c(Br)c(F)c3ncnc(c23)N2C[C@H]3CC[C@@H]([C@@H]12)N3C(=O)OC(C)(C)C]

Under nitrogen, to a solution of tert-butyl (1S,2S,5R)-2-((S)-1-((7-bromo-6-chloro-8-fluoro-4-hydroxyquinazolin-5-yl)oxy)ethyl)-3,8-diazabicyclo[3.2.1]octane-8-carboxylate (412 mg, 0.770 mmol) and DIPEA (300 mg, 2.32 mmol) in DCM (5 mL) was added BOPCl (987 mg, 3.87 mmol) at 0° C. and stirred at room temperature for 1 h. The resulting solution was concentrated under vacuum. The residue was purified by flash chromatography on silica gel (gradient: 0%-50% EtOAc/petroleum ether) to yield 171 mg (42.7% yield) of the title compound as a yellow solid. LC-MS: (ESI, m/z): [M+H]⁺=515.

### Step 3: tert-Butyl (5S,5aS,6S,9R)-2-(6-(bis(4-methoxybenzyl)amino)-4-methyl-3-(trifluoromethyl)pyridin-2-yl)-3-chloro-1-fluoro-5-methyl-5a,6,7,8,9,10-hexahydro-5H-6,9-epiminoazepino[2′,1′:3,4][1,4]oxazepino[5,6,7-de]quinazoline-15-carboxylate

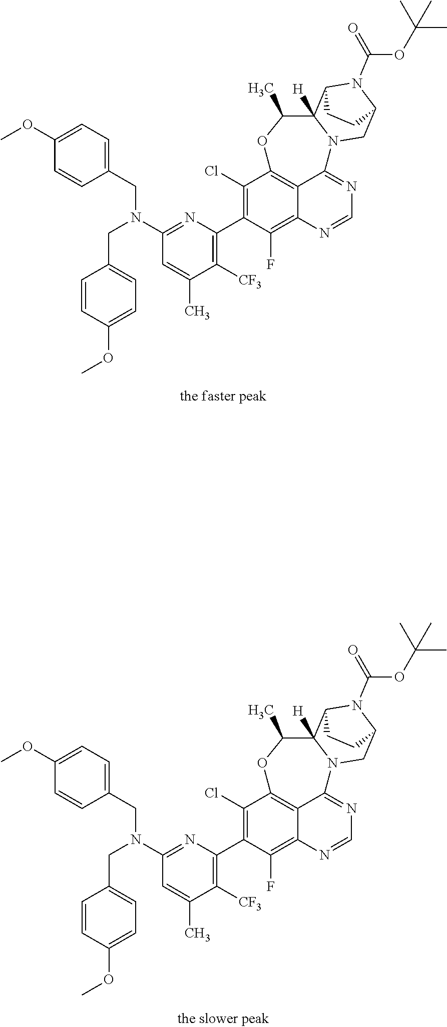

Under nitrogen, to a solution of tert-butyl (5S,5aS,6S,9R)-2-bromo-3-chloro-1-fluoro-5-methyl-5a,6,7,8,9,10-hexahydro-5H-6,9-epiminoazepino[2′,1′:3,4][1,4]oxazepino[5,6,7-de]quinazoline-15-carboxylate (149 mg, 0.290 mmol) in anhydrous THF (1 mL) was added i-PrMgCl·LiCl (0.3 mL, 1.3 M in THF) slowly at −78° C. The solution was stirred for 15 minutes at −78° C. Then ZnCl₂ (0.3 mL, 2 M in 2-Me THF) was added at −78° C. and stirred at −78° C. for 10 minutes. The mixture was warmed to room temperature and stirred for 20 minutes, the resulting solution was added into a degassed slurry of 6-bromo-N,N-bis(4-methoxybenzyl)-4-methyl-5-(trifluoromethyl)pyridin-2-amine (137 mg, 0.280 mmol, Intermediate 4) and Pd(PPh₃)₂Cl₂ (10.3 mg, 0.0100 mmol) in anhydrous THF (1 mL). The slurry was stirred overnight at 50° C. The solvent was concentrated under vacuum. The residue was purified by flash chromatography on silica gel (gradient: 0%-70% petroleum ether/EtOAc) to yield 99.2 mg (mixture of two atropisomers) of the title compound as a yellow solid. The mixture was separated by Chiral-HPLC (Column: CHIRALPAK ID, 2*25 cm, 5 μm; Mobile Phase A: Hex (0.5% 2M NH₃-MeOH)—HPLC, Mobile Phase B: IPA—HPLC; Flow rate: 20 mL/min; Gradient: 20% B to 20% B in 21 min; Wave Length: 220/254 nm; R_{T1} (min): 14.30; R_{T2} (min): 17.97; Sample Solvent: EtOH:DCM=1:1; Injection Volume: 0.5 mL; Number Of Runs: 7) to yield 45.1 mg (18.3% yield) of the faster peak and 53.2 mg (21.5% yield) of the slower peak as light yellow solids. LC-MS: (ESI, m/z): [M+H]⁺=849.

### Step 4: 6-((5S,5aS,6S,9R)-3-Chloro-1-fluoro-5-methyl-5a,6,7,8,9,10-hexahydro-5H-6,9-epiminoazepino[2′,1′:3,4][1,4]oxazepino[5,6,7-de]quinazolin-2-yl)-4-methyl-5-(trifluoromethyl)pyridin-2-amine

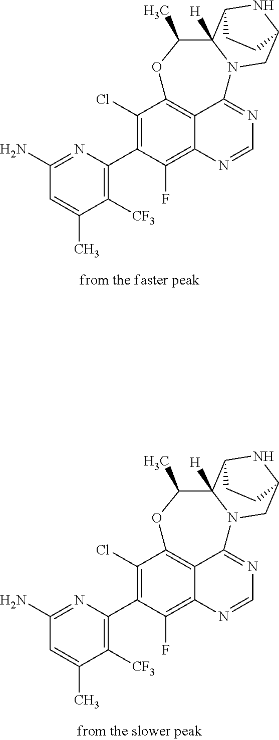 [SMILES: Cc1cc(N)nc(-c2c(Cl)c3c4c(ncnc4c2F)N2C[C@H]4CC[C@H](N4)[C@H]2[C@H](C)O3)c1C(F)(F)F ; Cc1cc(N)nc(-c2c(Cl)c3c4c(ncnc4c2F)N2C[C@H]4CC[C@H](N4)[C@H]2[C@H](C)O3)c1C(F)(F)F]

A solution of tert-butyl (5S,5aS,6S,9R)-2-(6-(bis(4-methoxybenzyl)amino)-4-methyl-3-(trifluoromethyl)pyridin-2-yl)-3-chloro-1-fluoro-5-methyl-5a,6,7,8,9,10-hexahydro-5H-6,9-epiminoazepino[2′,1′:3,4][1,4]oxazepino[5,6,7-de]quinazoline-15-carboxylate (45.1 mg, 0.0500 mmol) in TFA (2 mL) was stirred at 50° C. for 4 h. The solution was concentrated under vacuum. The residue was purified by Prep-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 26% B to 50% B in 8 min, 50% B; Wave Length: 254/220 nm; RT1 (min): 8) to yield 9.5 mg (34.8% yield) of Compound 42 as a white solid. LC-MS: (ESI, m/z): [M+H]⁺=509.1. ¹H NMR (400 MHz, DMSO-d₆, ppm) δ 8.52 (s, 1H), 6.84 (s, 2H), 6.46 (s, 1H), 5.25 (m, 1H), 4.36 (m, 1H), 3.99 (d, J=9.6 Hz, 1H), 3.54-3.48 (m, 1H), 3.41-3.35 (m, 1H), 2.99 (d, J=12.5 Hz, 1H), 2.35 (d, J=2.3 Hz, 3H), 1.90-1.80 (m, 1H), 1.66-1.51 (m, 2H), 1.51-1.38 (m, 4H)

A solution of tert-butyl (5S,5aS,6S,9R)-2-(6-(bis(4-methoxybenzyl)amino)-4-methyl-3-(trifluoromethyl)pyridin-2-yl)-3-chloro-1-fluoro-5-methyl-5a,6,7,8,9,10-hexahydro-5H-6,9-epiminoazepino[2′,1′:3,4][1,4]oxazepino[5,6,7-de]quinazoline-15-carboxylate (53.2 mg, 0.0600 mmol) in TFA (2 mL) was stirred at 50° C. for 4 h. The solution was concentrated under vacuum. The residue was purified by Prep-HPLC (Column: Xselect CSH C18 OBD Column 30*150 mm mm, 5 μm; Mobile Phase A: Water (0.1% FA), Mobile Phase B: ACN; Flow rate: 60 mL/min; Gradient: 15% B to 41% B in 8 min, 41% B; Wave Length: 254/220 nm; R_{T1} (min): 7) to yield 11.9 mg (37.4% yield) of Compound 42 as a white solid. LC-MS: (ESI, m/z): [M+H]⁺=509.1. ¹H NMR (400 MHz, DMSO-d₆, ppm) δ 8.53 (s, 1H), 6.83 (s, 2H), 6.47 (s, 1H), 5.27 (m, 1H), 4.37 (m, 1H), 4.05 (d, J=9.7 Hz, 1H), 3.61 (s, 1H), 3.50-3.44 (m, 1H), 3.04 (d, J=12.8 Hz, 1H), 2.51-2.37 (m, 3H), 1.93 (s, 1H), 1.73-1.53 (m, 3H), 1.52-1.40 (m, 3H).

### Example 43: Compound 43A and 43B: (5S,5aS,6S,9R)-3-Chloro-1-fluoro-2-(6-fluoro-1-methyl-1H-indazol-7-yl)-13-(((2R,7aS)-2-fluorotetrahydro-1H-pyrrolizin-7a(5H)-yl)methoxy)-5-methyl-5a,6,7,8,9,10-hexahydro-5H-6,9-epiminoazepino[2′,1′:3,4][1,4]oxazepino[5,6,7-de]quinazoline

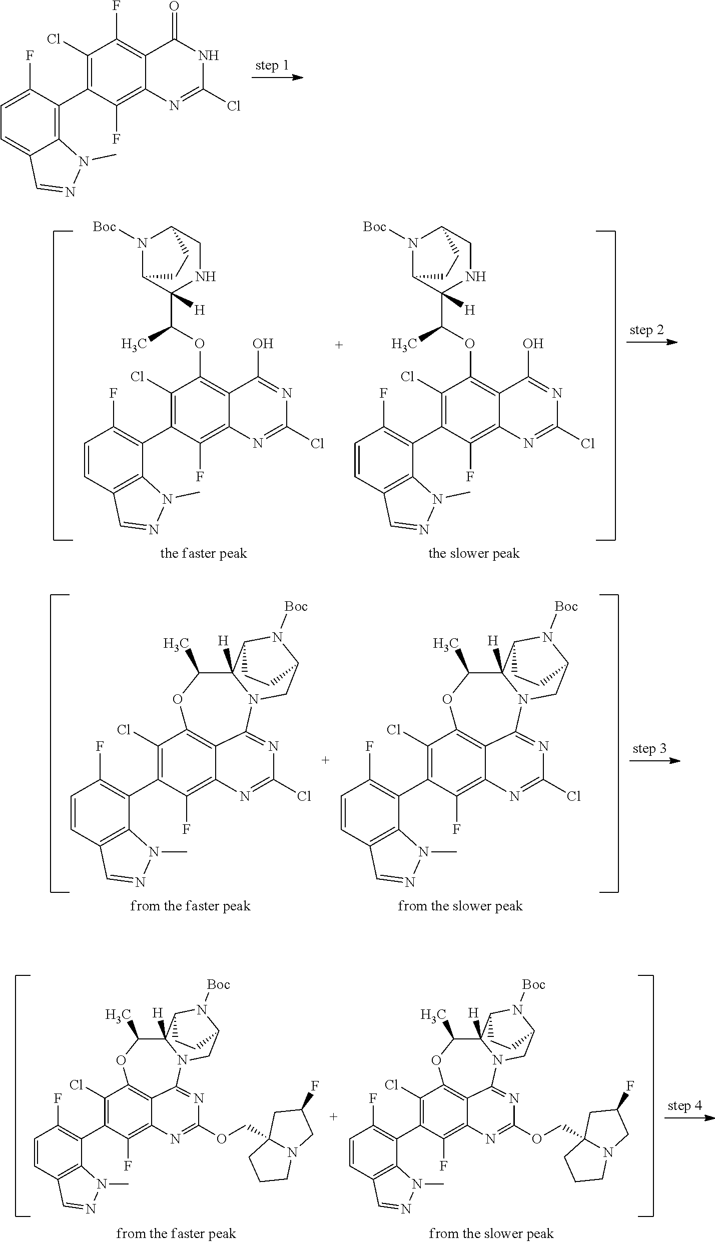

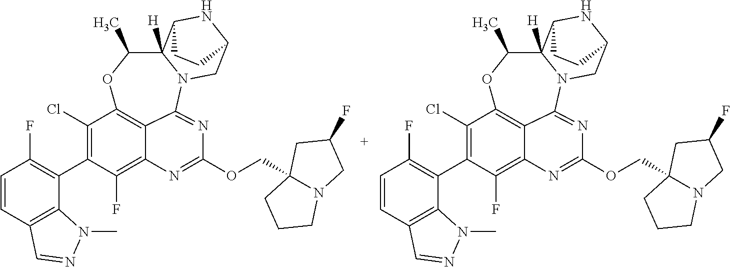

### Step 1: tert-Butyl (1S,2S,5R)-2-((1S)-1-((2,6-dichloro-8-fluoro-7-(6-fluoro-1-methyl-1H-indazol-7-yl)-4-hydroxyquinazolin-5-yl)oxy)ethyl)-3,8-diazabicyclo[3.2.1]octane-8-carboxylate

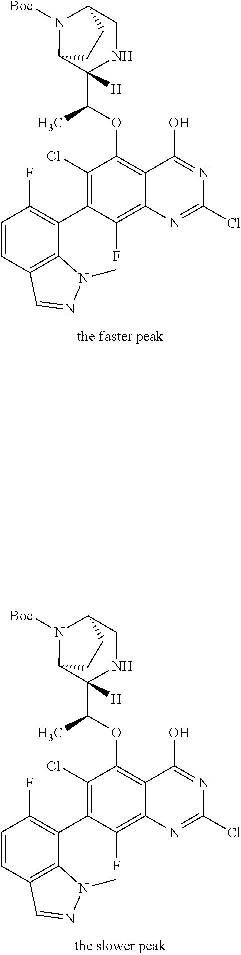 [SMILES: C[C@H](Oc1c(Cl)c(-c2c(F)ccc3cnn(C)c23)c(F)c2nc(Cl)nc(O)c12)[C@H]1NC[C@H]2CC[C@@H]1N2C(=O)OC(C)(C)C ; C[C@H](Oc1c(Cl)c(-c2c(F)ccc3cnn(C)c23)c(F)c2nc(Cl)nc(O)c12)[C@H]1NC[C@H]2CC[C@@H]1N2C(=O)OC(C)(C)C]

Under nitrogen, a solution of tert-butyl (1R,2S,5S)-2-((S)-1-hydroxyethyl)-3,8-diazabicyclo[3.2.1]octane-8-carboxylate (160 mg, 0.620 mmol, Intermediate 29) in THF (5 mL) was added NaH (75.0 mg, 1.88 mmol, 60% in mineral oil) at 0° C. The resulting solution was stirred for 30 min at 0° C. Then 2,6-dichloro-5,8-difluoro-7-(6-fluoro-1-methyl-1H-indazol-7-yl)quinazolin-4 (3H)-one (250 mg, 0.630 mmol, Intermediate 10) was added and stirred at room temperature for 4 hours. The reaction was quenched with aqueous NH₄Cl and the mixture was extracted with EtOAc. The combined organic layers were dried over anhydrous sodium sulfate and concentrated under vacuum. The residue was purified by flash chromatography on silica gel eluting (gradient: 0%-10% MeCOH/DCM) to yield 122 mg of the faster peak and 114 mg of the slower peak as white solids. LC-MS: (ESI, m/z): [M+H]⁺=635.

### Step 2: tert-Butyl (5S,5aS,6S,9R)-3,13-dichloro-1-fluoro-2-(6-fluoro-1-methyl-1H-indazol-7-yl)-5-methyl-5a,6,7,8,9,10-hexahydro-5H-6,9-epiminoazepino[2′,1′:3,4][1,4]oxazepino[5,6,7-de]quinazoline-15-carboxylate

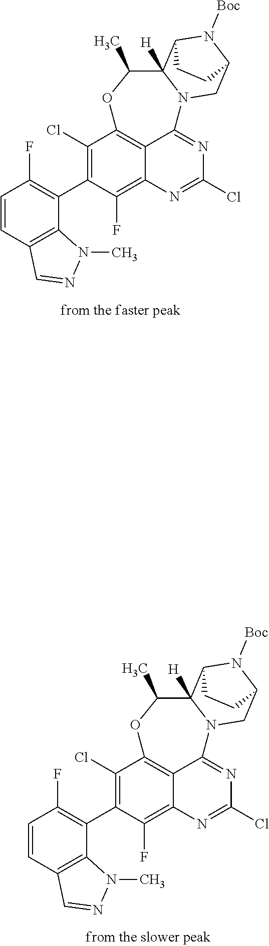 [SMILES: C[C@@H]1Oc2c(Cl)c(-c3c(F)ccc4cnn(C)c34)c(F)c3nc(Cl)nc(c23)N2C[C@H]3CC[C@@H]([C@@H]12)N3C(=O)OC(C)(C)C ; C[C@@H]1Oc2c(Cl)c(-c3c(F)ccc4cnn(C)c34)c(F)c3nc(Cl)nc(c23)N2C[C@H]3CC[C@@H]([C@@H]12)N3C(=O)OC(C)(C)C]

A solution of (1S,2S,5R)-2-((1S)-1-((2,6-dichloro-8-fluoro-7-(6-fluoro-1-methyl-1H-indazol-7-yl)-4-hydroxyquinazolin-5-yl)oxy)ethyl)-3,8-diazabicyclo[3.2.1]octane-8-carboxylate (104 mg, 0.170 mmol), BOPCl (210 mg, 0.830 mmol) and DIPEA (64.1 mg, 0.500 mmol) in DCM (5 mL) was stirred at room temperature for 1 hour. The solvent was concentrated under vacuum. The residue was purified by flash chromatography on silica gel eluting (gradient: 0%-50% EtOAc/petroleum ether) to afford 90.3 mg (88.6% yield) of the title compound as a yellow solid. LC-MS: (ESI, m/z): [M+H]⁺=617.

Analogous to the method described above, the other isomer (83.3 mg) was prepared from tert-butyl 3-((S)-9-bromo-8-chloro-5-(cyanomethyl)-5,6-dihydro-4H-[1,4]oxazepino[5,6,7-de]quinazolin-4-yl)pyrrolidine-1-carboxylate (the slower peak of step 1).

### Step 3: tert-Butyl (5S,5aS,6S,9R)-3-chloro-1-fluoro-2-(6-fluoro-1-methyl-1H-indazol-7-yl)-13-(((2R,7aS)-2-fluorotetrahydro-1H-pyrrolizin-7a(5H)-yl)methoxy)-5-methyl-5a,6,7,8,9,10-hexahydro-5H-6,9-epiminoazepino[2′,1′:3,4][1,4]oxazepino[5,6,7-de]quinazoline-15-carboxylate

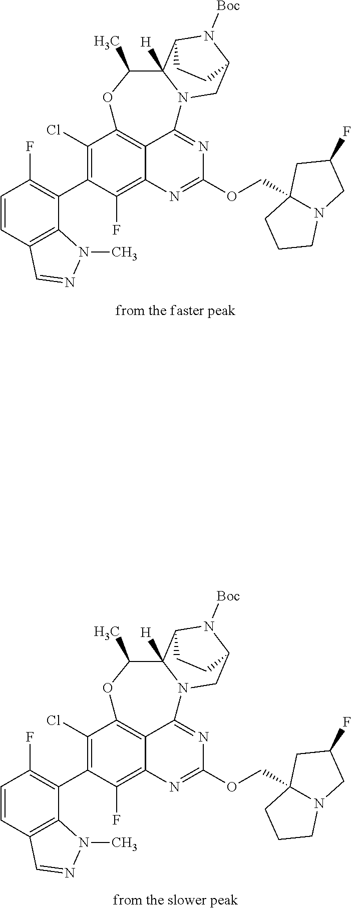 [SMILES: C[C@@H]1Oc2c(Cl)c(-c3c(F)ccc4cnn(C)c34)c(F)c3nc(OC[C@@]45CCCN4C[C@H](F)C5)nc(c23)N2C[C@H]3CC[C@@H]([C@@H]12)N3C(=O)OC(C)(C)C ; C[C@@H]1Oc2c(Cl)c(-c3c(F)ccc4cnn(C)c34)c(F)c3nc(OC[C@@]45CCCN4C[C@H](F)C5)nc(c23)N2C[C@H]3CC[C@@H]([C@@H]12)N3C(=O)OC(C)(C)C]

Under nitrogen, to a solution of ((2R,7aS)-2-fluorotetrahydro-1H-pyrrolizin-7a(5H)-yl)methanol (27.5 mg, 0.170 mmol, Intermediate 16) in THF (3 mL) was added NaH (13.9 mg, 0.350 mmol, 60% suspend in oil) at 0° C. The resulting solution was stirred for 30 min at 0° C. Then tert-butyl (5S,5aS,6S,9R)-3,13-dichloro-1-fluoro-2-(6-fluoro-1-methyl-1H-indazol-7-yl)-5-methyl-5a,6,7,8,9,10-hexahydro-5H-6,9-epiminoazepino[2′,1′:3,4][1,4]oxazepino[5,6,7-de]quinazoline-15-carboxylate (71.1 mg, 0.120 mmol) was added and stirred at room temperature for 4 hours. The reaction was quenched with aqueous NH₄Cl. The resulting solution was extracted with EtOAc (30 mL). The combined organic layers were dried over anhydrous sodium sulfate and concentrated under vacuum. The residue was purified by flash chromatography on silica gel eluting (gradient: 0%-10% MeCOH/DCM) to afford 53 mg (62.2% yield) of the title compound as a yellow solid. LC-MS: (ESI, m/z): [M+H]⁺=740.

Analogous to the method described above, the other isomer (62.0 mg) was prepared from tert-butyl (5S,5aS,6S,9R)-3,13-dichloro-1-fluoro-2-(6-fluoro-1-methyl-1H-indazol-7-yl)-5-methyl-5a,6,7,8,9,10-hexahydro-5H-6,9-epiminoazepino[2′,1′:3,4][1,4]oxazepino[5,6,7-de]quinazoline-15-carboxylate (the slower peak of step 2).

### Step 4: (5S,5aS,6S,9R)-3-Chloro-1-fluoro-2-(6-fluoro-1-methyl-1H-indazol-7-yl)-13-(((2R,7aS)-2-fluorotetrahydro-1H-pyrrolizin-7a(5H)-yl)methoxy)-5-methyl-5a,6,7,8,9,10-hexahydro-5H-6,9-epiminoazepino[2′,1′:3,4][1,4]oxazepino[5,6,7-de]quinazoline

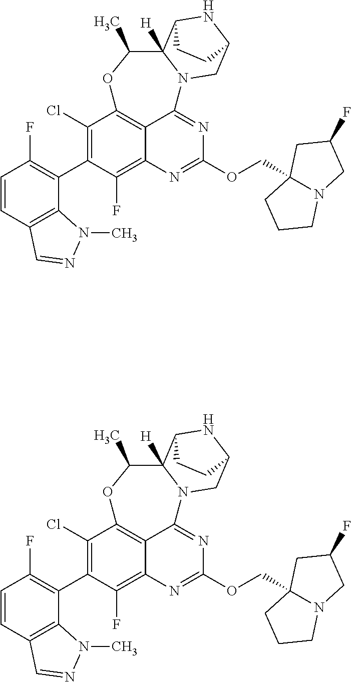 [SMILES: C[C@@H]1Oc2c(Cl)c(-c3c(F)ccc4cnn(C)c34)c(F)c3nc(OC[C@@]45CCCN4C[C@H](F)C5)nc(c23)N2C[C@H]3CC[C@H](N3)[C@@H]12 ; C[C@@H]1Oc2c(Cl)c(-c3c(F)ccc4cnn(C)c34)c(F)c3nc(OC[C@@]45CCCN4C[C@H](F)C5)nc(c23)N2C[C@H]3CC[C@H](N3)[C@@H]12]

Under nitrogen, to a solution of tert-butyl (5S,5aS,6S,9R)-3-chloro-1-fluoro-2-(6-fluoro-1-methyl-1H-indazol-7-yl)-13-(((2R,7aS)-2-fluorotetrahydro-1H-pyrrolizin-7a(5H)-yl)methoxy)-5-methyl-5a,6,7,8,9,10-hexahydro-5H-6,9-epiminoazepino[2′,1′:3,4][1,4]oxazepino[5,6,7-de]quinazoline-15-carboxylate (53.0 mg, 0.0700 mmol) in DCM (2 mL) was added TFA (0.5 mL). The result solution was stirred for 40 min at room temperature. The solvent was concentrated under vacuum. The residue was purified by C18 column (solvent gradient: 0-48% ACN in water (0.05% NH₄HCO₃)) to yield 19.4 mg (42.3% yield) of Compound 43 as a white solid. LC-MS: (ESI, m/z): [M+H]⁺=640. ¹H NMR (400 MHz, DMSO-d₆, ppm): δ 8.19 (s, 1H), 7.97 (dd, J=8.9, 5.2 Hz, 1H), 7.21 (t, J=9.3 Hz, 1H), 5.35-5.19 (m, 2H), 4.46 (dd, J=9.4, 6.2 Hz, 1H), 4.09-3.99 (m, 3H), 3.60 (s, 4H), 3.58 (d, J=5.3 Hz, 1H), 3.12-2.98 (m, 4H), 2.83 (t, J=8.1 Hz, 1H), 2.20-2.10 (m, 3H), 1.93-1.84 (m, 4H), 1.52 (d, J=6.2 Hz, 6H).

Analogous to the method described above, the other isomer (35.1 mg, 72.5% yield) of Compound 43 was prepared from tert-butyl (5S,5aS,6S,9R)-3-chloro-1-fluoro-2-(6-fluoro-1-methyl-1H-indazol-7-yl)-13-(((2R,7aS)-2-fluorotetrahydro-1H-pyrrolizin-7a(5H)-yl)methoxy)-5-methyl-5a,6,7,8,9,10-hexahydro-5H-6,9-epiminoazepino[2′,1′:3,4][1,4]oxazepino[5,6,7-de]quinazoline-15-carboxylate (the slower peak of step 3). LC-MS: (ESI, m/z): [M+H]⁺=640. ¹H NMR (400 MHz, DMSO-d₆, ppm): δ 8.17 (s, 1H), 7.97 (dd, J=8.8, 5.2 Hz, 1H), 7.23 (dd, J=9.7, 8.8 Hz, 1H), 5.35 (d, J=3.9 Hz, 1H), 5.15 (dd, J=12.7, 2.7 Hz, 1H), 4.51 (dd, J=9.8, 6.2 Hz, 1H), 4.11-3.96 (m, 3H), 3.57 (d, J=5.7 Hz, 1H), 3.46 (s, 4H), 3.12-2.99 (m, 4H), 2.82 (dd, J=8.3, 7.8 Hz, 1H), 2.21-2.11 (m, 1H), 2.05 (d, J=3.1 Hz, 1H), 2.00 (s, 1H), 1.93-1.82 (dd, J=11.8, 8.0 Hz, 2H), 1.79 (dd, J=13.2, 6.0 Hz, 2H), 1.65 (dd, J=11.8, 8.0 Hz, 2H), 1.65-1.47 (m, 4H).

### Example 44: Compound 44: 2-((5S,5aS,6S,9R)-3-Chloro-1-fluoro-13-(((2R,7aS)-2-fluorotetrahydro-1H-pyrrolizin-7a(5H)-yl)methoxy)-5-methyl-5a,6,7,8,9,10-hexahydro-5H-6,9-epiminoazepino[2′,1′:3,4][1,4]oxazepino[5,6,7-de]quinazolin-2-yl)phenol

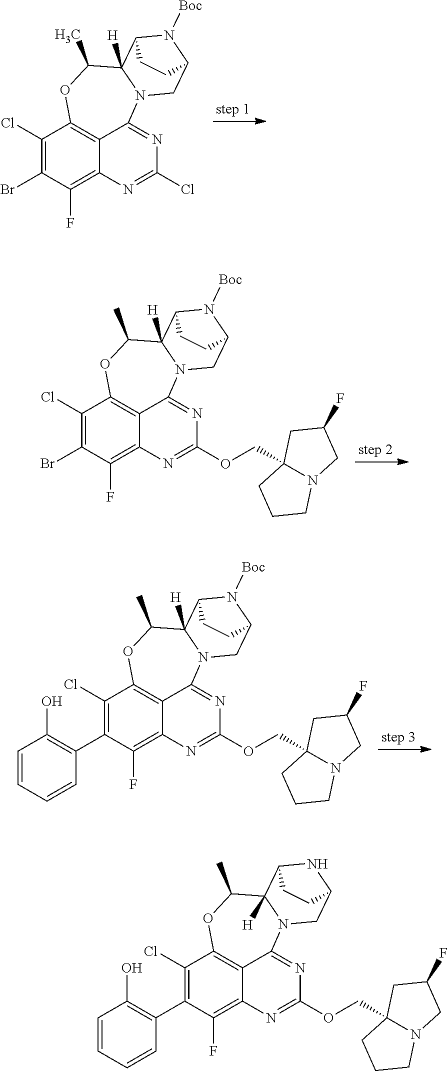

### Step 1: tert-Butyl (5S,5aS,6S,9R)-2-bromo-3-chloro-1-fluoro-13-(((2R,7aS)-2-fluorotetrahydro-1H-pyrrolizin-7a(5H)-yl)methoxy)-5-methyl-5a,6,7,8,9,10-hexahydro-5H-6,9-epiminoazepino[2′,1′:3,4][1,4]oxazepino[5,6,7-de]quinazoline-15-carboxylate

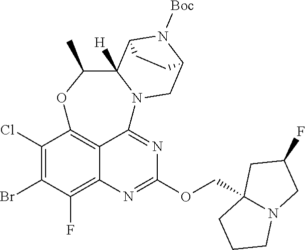 [SMILES: C[C@@H]1Oc2c(Cl)c(Br)c(F)c3nc(OC[C@@]45CCCN4C[C@H](F)C5)nc(c23)N2C[C@H]3CC[C@@H]([C@@H]12)N3C(=O)OC(C)(C)C]

Under nitrogen, to a solution of ((2R,7aS)-2-fluorotetrahydro-1H-pyrrolizin-7a(5H)-yl)methanol (80.0 mg, 0.500 mmol, intermediate 10) in THF (10 mL) was added NaH (40.0 mg, 1.00 mmol, 60% in mineral oil) at 0° C. The resulting solution was stirred at room temperature for 0.5 h. Then tert-butyl (5S,5aS,6S,9R)-2-bromo-3,13-dichloro-1-fluoro-5-methyl-5a,6,7,8,9,10-hexahydro-5H-6,9-epiminoazepino[2′,1′:3,4][1,4]oxazepino[5,6,7-de]quinazoline-15-carboxylate (240 mg, 0.438 mmol, intermediate 8, step 2) was added at 0° C. and stirred at room temperature for 1 h. The reaction was quenched with NH₄Cl (aq.) and concentrated under vacuum. The residue was purified by flash chromatography on silica gel eluting with EtOAc/petroleum ether (0-10%) to afford the title compound 240 mg (81.6% yield) as a white solid. LC-MS: (ESI, m/z): [M+H]⁺=670/672

### Step 2: tert-Butyl (5S,5aS,6S,9R)-3-chloro-1-fluoro-13-(((2R,7aS)-2-fluorotetrahydro-1H-pyrrolizin-7a(5H)-yl)methoxy)-2-(2-hydroxyphenyl)-5-methyl-5a,6,7,8,9,10-hexahydro-5H-6,9-epiminoazepino[2′,1′:3,4][1,4]oxazepino[5,6,7-de]quinazoline-15-carboxylate

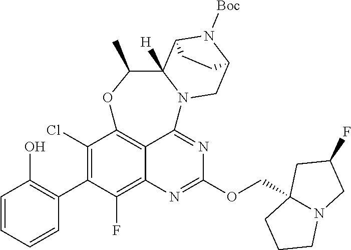 [SMILES: C[C@@H]1Oc2c(Cl)c(-c3ccccc3O)c(F)c3nc(OC[C@@]45CCCN4C[C@H](F)C5)nc(c23)N2C[C@H]3CC[C@@H]([C@@H]12)N3C(=O)OC(C)(C)C]

Under nitrogen, a solution of tert-butyl (5S,5aS,6S,9R)-2-bromo-3-chloro-1-fluoro-13-(((2R,7aS)-2-fluorotetrahydro-1H-pyrrolizin-7a(5H)-yl)methoxy)-5-methyl-5a,6,7,8,9,10-hexahydro-5H-6,9-epiminoazepino[2′,1:3,4][1,4]oxazepino[5,6,7-de]quinazoline-15-carboxylate (230 mg, 0.343 mmol), (2-hydroxyphenyl)boronic acid (47.4 mg, 0.343 mmol), Pd(PPh₃)₄ (39.7 mg, 0.0343 mmol) and K₂CO₃ (94.8 mg, 0.687 mmol) in DME (10 mL) and water (1 mL) was stirred at 90° C. for 5 hours. The reaction system was cooled to room temperature, diluted with water and extracted with EtOAc. The combined organic layers were dried over anhydrous Na₂SO₄ and concentrated under vacuum. The residue was purified by flash chromatography on silica gel eluting with EtOAc/petroleum ether (0-20%) to afford 160 mg (68.2% yield) of the title compound as a yellow solid. LC-MS: (ESI, m/z): [M+H]⁺=684.

### Step 3: 2-((5S,5aS,6S,9R)-3-Chloro-1-fluoro-13-(((2R,7aS)-2-fluorotetrahydro-1H-pyrrolizin-7a(5H)-yl)methoxy)-5-methyl-5a,6,7,8,9,10-hexahydro-5H-6,9-epiminoazepino[2′,1′:3,4][1,4]oxazepino[5,6,7-de]quinazolin-2-yl)phenol

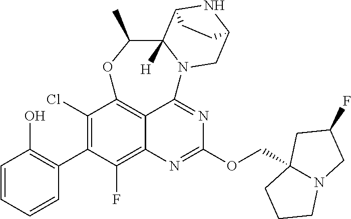

To a solution of tert-butyl (5S,5aS,6S,9R)-3-chloro-1-fluoro-13-(((2R,7aS)-2-fluorotetrahydro-1H-pyrrolizin-7a(5H)-yl)methoxy)-2-(2-hydroxyphenyl)-5-methyl-5a,6,7,8,9,10-hexahydro-5H-6,9-epiminoazepino[2′,1′:3,4][1,4]oxazepino[5,6,7-de]quinazoline-15-carboxylate (100.0 mg, 0.150 mmol) in DCM (3 mL) was added TFA (0.6 mL). The solution was stirred at room temperature for 1 hour. Concentrated under vacuum. The residue was purified by Prep-HPLC with the following conditions: (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 25% B to 48% B in 10 min, 48% B; Wave Length: 254/220 nm; RT (min): 9.6) to afford the title compound 27 mg (31.6% yield) as a white solid. LC-MS: (ESI, m/z): [M+H]⁺=584. ¹H NMR (300 MHz, DMSO-d₆) δ 9.62 (d, J=14.5 Hz, 1H), 7.28 (t, J=8.6 Hz, 1H), 7.10 (dd, J=20.0, 7.6 Hz, 1H), 7.02-6.84 (m, 2H), 5.38-5.19 (d, J=57 Hz, 1H), 5.18-5.10 (m, 1H), 4.30-4.25 (m, 1H), 4.05 (dd, J=30, 10.5 Hz, 2H), 4.04-3.94 (m, 1H), 3.57 (d, J=5.4 Hz, 1H), 3.41 (d, J=4.8 Hz, 1H), 3.19-2.97 (m, 4H), 2.90-2.75 (m, 1H), 2.25-1.50 (m, 13H).

### Example 45: Compound 45A and 45B: 6-((5aS,6R,9R)-3-Chloro-1-fluoro-15-(3-fluoropropyl)-13-(((2R,7aS)-2-fluorotetrahydro-1H-pyrrolizin-7a(5H)-yl)methoxy)-5a,6,7,8,9,10-hexahydro-5H-9,6-(epiminomethano)azepino[2′,1′:3,4][1,4]oxazepino[5,6,7-de]quinazolin-2-yl)-4-methyl-5-(trifluoromethyl)pyridin-2-amine (two atropisomers)

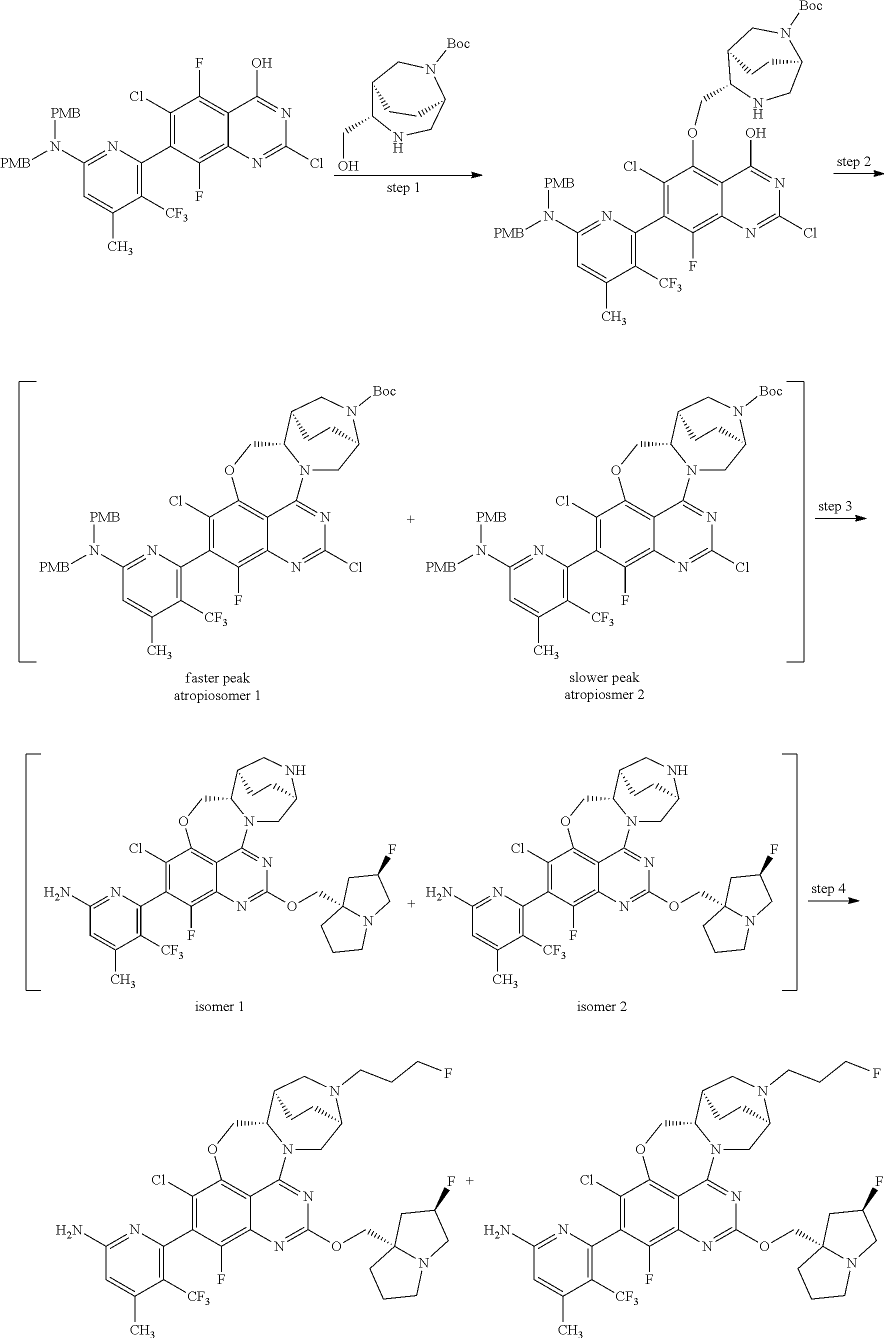

### Step 1: tert-Butyl (1R,2S,5R)-2-(((7-(6-(bis(4-methoxybenzyl)amino)-4-methyl-3-(trifluoromethyl)pyridin-2-yl)-2,6-dichloro-8-fluoro-4-hydroxyquinazolin-5-yl)oxy)methyl)-3,6-diazabicyclo[3.2.2]nonane-6-carboxylate

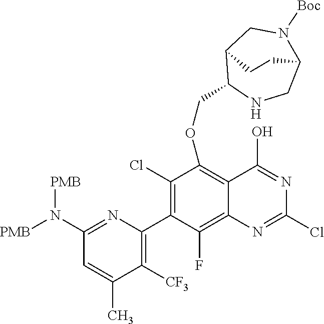

Under nitrogen, to a solution of tert-butyl (1R,2S,5R)-2-(hydroxymethyl)-3,6-diazabicyclo[3.2.2]nonane-6-carboxylate (120 mg, 0.470 mmol, intermediate 38) in tetrahydrofuran (10 mL) was added NaH (210 mg, 5.25 mmol, 60% in mineral oil) at 0° C. The resulting solution was stirred at room temperature for 30 min at 0° C. Then 7-(6-(bis(4-methoxybenzyl)amino)-4-methyl-3-(trifluoromethyl)pyridin-2-yl)-2,6-dichloro-5,8-difluoroquinazolin-4-ol (350 mg, 0.530 mmol, intermediate 4) was added at 0° C. and stirred at 40° C. for 24 hours. The reaction was quenched with aqueous NH₄Cl solution and extracted with EtOAc. The combined organic layers were dried over anhydrous sodium sulfate and concentrated under vacuum. The residue was purified by flash chromatography on silica gel (gradient: 0-20% MeCOH in DCM) to afford 130 mg of the title compound as a yellow solid. LC-MS: (ESI, m/z): [M+H]⁺=901.

### Step 2: tert-Butyl (5aS,6R,9R)-2-(6-(bis(4-methoxybenzyl)amino)-4-methyl-3-(trifluoromethyl)pyridin-2-yl)-3,13-dichloro-1-fluoro-5a,6,7,8,9,10-hexahydro-5H-9,6-(epiminomethano)azepino[2′,1′:3,4][1,4]oxazepino[5,6,7-de]quinazoline-15-carboxylate

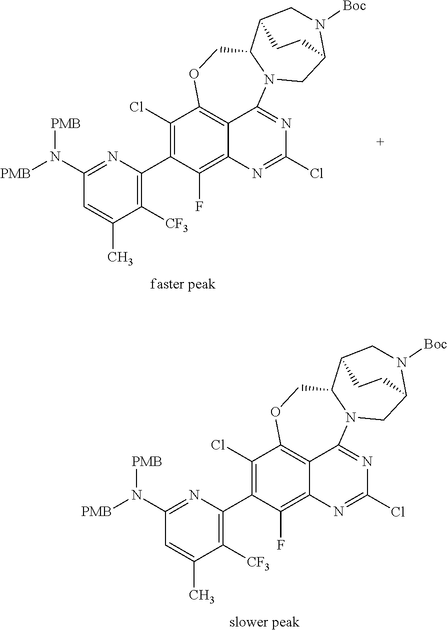

A solution of tert-butyl (1R,2S,5R)-2-(((7-(6-(bis(4-methoxybenzyl)amino)-4-methyl-3-(trifluoromethyl)pyridin-2-yl)-2,6-dichloro-8-fluoro-4-hydroxyquinazolin-5-yl)oxy)methyl)-3,6-diazabicyclo[3.2.2]nonane-6-carboxylate (95.0 mg, 0.105 mmol), BOPCl (107 mg, 0.421 mmol) and DIPEA (204 mg, 1.58 mmol) in DCM (3 mL) was stirred at room temperature for 12 hours. The reaction system was diluted with water and extracted with DCM. The combined organic layers were dried over anhydrous Na₂SO₄ and concentrated under vacuum. The residue was purified by reverse phase flash chromatography on pre-packed C18 column (gradient: 0-100% CH₃CN in water (0.05% NH₄HCO₃)) to yield 50 mg of mixture of atropisomers as a white solid. The atropisomers were separated by Chiral-Prep-HPLC with the following conditions: (Column: CHIRAL ART Cellulose-SB, 2*25 cm, 5 μm; Mobile Phase A: Hex (0.5% 2M NH₃-MeOH), Mobile Phase B: IPA; Flow rate: 20 mL/min; Gradient: 20% B to 20% B in 49 min; Wavelength: 220/254 nm; R_{T1} (min): 30.793; R_{T2} (min): 41.647) to yield 30 mg of the faster peak and 35 mg of the slower peak as white solids. LC-MS: (ESI, m/z): [M+H]⁺=883.

### Step 3: 6-((5aS,6R,9R)-3-Chloro-1-fluoro-13-(((2R,7aS)-2-fluorotetrahydro-1H-pyrrolizin-7a(5H)-yl)methoxy)-5a,6,7,8,9,10-hexahydro-5H-9,6-(epiminomethano)azepino[2′,1′:3,4][1,4]oxazepino[5,6,7-de]quinazolin-2-yl)-4-methyl-5-(trifluoromethyl)pyridin-2-amine

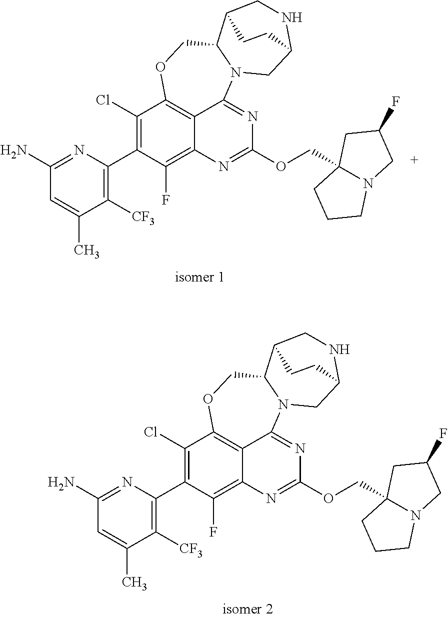

A solution of tert-butyl (5aS,6R,9R)-2-(6-(bis(4-methoxybenzyl)amino)-4-methyl-3-(trifluoromethyl)pyridin-2-yl)-3,13-dichloro-1-fluoro-5a,6,7,8,9,10-hexahydro-5H-9,6-(epiminomethano)azepino[2′,1′:3,4][1,4]oxazepino[5,6,7-de]quinazoline-15-carboxylate (30.0 mg, 0.0339 mmol) (from faster peak) in TFA (1 mL) was stirred at 50° C. for 8 hours. The reaction mixture was concentrated under vacuum. The residue was purified by reverse phase flash chromatography on pre-packed C18 column (gradient: 0-100% CH₃CN in water (0.05% NH₄HCO₃)) to yield 15.0 mg of the title compound (isomer 1) as a white solid. LC-MS: (ESI, m/z): [M+H]⁺=666.

Analogous to method described as above, the other isomer (isomer 2) 20.0 mg was prepared from tert-butyl (5aS,6R,9R)-2-(6-(bis(4-methoxybenzyl)amino)-4-methyl-3-(trifluoromethyl)pyridin-2-yl)-3,13-dichloro-1-fluoro-5a,6,7,8,9,10-hexahydro-5H-9,6-(epiminomethano)azepino[2′,1′:3,4][1,4]oxazepino[5,6,7-de]quinazoline-15-carboxylate (35.0 mg, 0.0339 mmol) as a white solid. LC-MS: (ESI, m/z): [M+H]⁺=666.

### Step 4: 6-((5aS,6R,9R)-3-Chloro-1-fluoro-15-(3-fluoropropyl)-13-(((2R,7aS)-2-fluorotetrahydro-1H-pyrrolizin-7a(5H)-yl)methoxy)-5a,6,7,8,9,10-hexahydro-5H-9,6-(epiminomethano)azepino[2′,1′:3,4][1,4]oxazepino[5,6,7-de]quinazolin-2-yl)-4-methyl-5-(trifluoromethyl)pyridin-2-amine

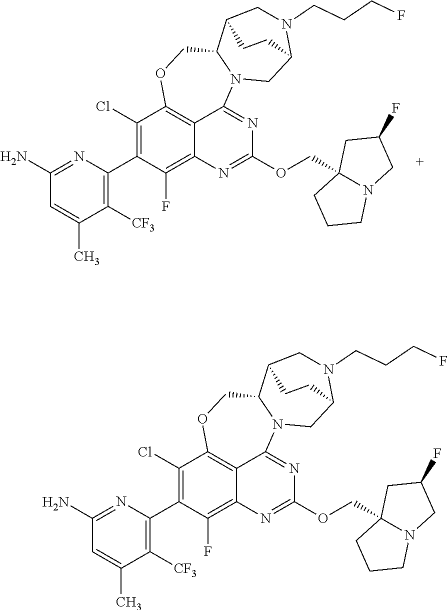

Under nitrogen, a solution of 6-((5aS,6R,9R)-3-Chloro-1-fluoro-13-(((2R,7aS)-2-fluorotetrahydro-1H-pyrrolizin-7a(5H)-yl)methoxy)-5a,6,7,8,9,10-hexahydro-5H-9,6-(epiminomethano)azepino[2′,1′:3,4][1,4]oxazepino[5,6,7-de]quinazolin-2-yl)-4-methyl-5-(trifluoromethyl)pyridin-2-amine (15.0 mg, 0.0225 mmol, isomer 1 of last step), 1-iodo-3-fluoropropane (10.0 mg, 0.0500 mmol) and DIPEA (40.0 mg, 0.310 mmol) in acetonitrile (4 mL) was stirred at 40° C. for 12 hours. The crude product was purified by Prep-HPLC with the following conditions: (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 30% B to 60% B in 10 min, 60% B; Wavelength: 254/220 nm; R_{T} (min): 9.6) to yield 5.8 mg (35% yield) of Compound 45 as a while solid. LC-MS: (ESI, m/z): [M+H]⁺=726. ¹H NMR (300 MHz, DMSO-d6, ppm) δ6.81 (s, 2H), 6.50-6.43 (m, 1H), 5.40-5.15 (m, 1H), 4.89 (dd, J=13.6, 5.4 Hz, 1H), 4.60 (dt, J=12.7, 5.3 Hz, 2H), 4.51-4.41 (m, 2H), 4.22 (s, 1H), 4.07 (d, J=10.3 Hz, 1H), 3.95 (d, J=10.3 Hz, 1H), 3.26 (s, 1H), 3.18-2.96 (m, 5H), 2.90-2.75 (m, 1H), 2.69 (t, J=6.9 Hz, 2H), 2.60-2.50 (m, 1H), 2.39-2.31 (m, 4H), 2.22-2.09 (m, 1H), 2.09-1.96 (m, 2H), 1.90-1.70 (m, 6H), 1.57-1.43 (m, 2H), 1.41-1.31 (m, 1H).

Analogous to method described as above, the other isomer (Compound 45) 7.1 mg (32% yield) was prepared from 6-((5aS,6R,9R)-3-Chloro-1-fluoro-13-(((2R,7aS)-2-fluorotetrahydro-1H-pyrrolizin-7a(5H)-yl)methoxy)-5a,6,7,8,9,10-hexahydro-5H-9,6-(epiminomethano)azepino[2′,1′:3,4][1,4]oxazepino[5,6,7-de]quinazolin-2-yl)-4-methyl-5-(trifluoromethyl)pyridin-2-amine (20.0 mg, 0.0300 mmol, isomer 2 of last step). LC-MS: (ESI, m/z): [M+H]⁺=726. ¹H NMR (400 MHz, DMSO-d6, ppm) δ 6.82 (s, 2H), 6.47 (s, 1H), 5.40-5.20 (m, 1H), 4.96 (dd, J=13.7, 5.3 Hz, 1H), 4.66-4.57 (m, 2H), 4.49 (t, J=6.0 Hz, 1H), 4.42 (dd, J=13.1, 1.7 Hz, 1H), 4.22 (d, J=4.0 Hz, 1H), 4.13 (d, J=10.4 Hz, 1H), 3.95 (d, J=10.4 Hz, 1H), 3.35-3.25 (m, 1H), 3.15-3.00 (m, 5H), 2.84 (q, J=8.3 Hz, 1H), 2.71 (t, J=6.9 Hz, 2H), 2.60-2.50 (m, 1H), 2.40-2.33 (m, 4H), 2.21-2.10 (m, 1H), 2.10-1.90 (m, 2H), 1.90-1.70 (m, 6H), 1.61-1.42 (m, 2H), 1.42-1.35 (m, 1H).

### Example 46: Compound 46: 6-((2R,5aS,6R,9R)-3-Chloro-1-fluoro-13-(((2R,7aS)-2-fluorotetrahydro-1H-pyrrolizin-7a(5H)-yl)methoxy)-15-methyl-5a,6,7,8,9,10-hexahydro-5H-9,6-(epiminomethano)azepino[2′,1′:3,4][1,4]oxazepino[5,6,7-de]quinazolin-2-yl)-4-methyl-5-(trifluoromethyl)pyridin-2-amine

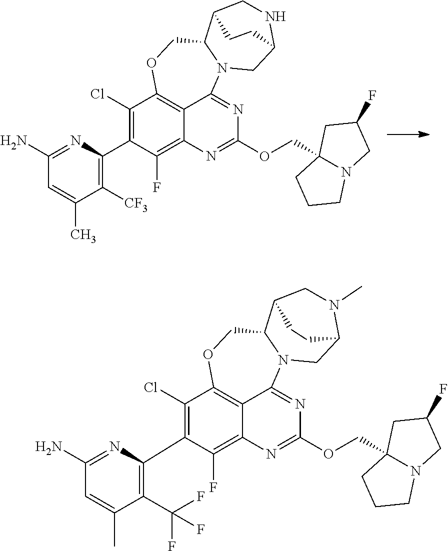

Under nitrogen, a solution of 6-((5aS,6R,9R)-3-Chloro-1-fluoro-13-(((2R,7aS)-2-fluorotetrahydro-1H-pyrrolizin-7a(5H)-yl)methoxy)-5a,6,7,8,9,10-hexahydro-5H-9,6-(epiminomethano)azepino[2′,1′:3,4][1,4]oxazepino[5,6,7-de]quinazolin-2-yl)-4-methyl-5-(trifluoromethyl)pyridin-2-amine (20.0 mg, 0.0300 mmol, Example 45, step 3, isomer 2), AcOH (2.5 mg, 0.0300 mmol), HCHO (0.05 mL, 40% in water) in methyl alcohol (1 mL) was stirred at room temperature for 1 h. Then NaBH₃CN (5.7 mg, 0.0900 mmol) was added and stirred at room temperature for 1 hour. The reaction system was concentrated under vacuum. The crude product was purified by Prep-HPLC with the following conditions: (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 34% B to 60% B in 9 min, 60% B; Wavelength: 254/220 nm; R_{T} (min): 8.85) to yield 3.0 mg of the title compound as a white solid. LC-MS: (ESI, m/z): [M+H]⁺=680. ¹H NMR (300 MHz, DMSO-d6, ppm) δ 6.83 (s, 2H), 6.48 (s, 1H), 5.42-5.15 (m, 1H), 4.93 (dd, J=13.6, 5.5 Hz, 1H), 4.61 (dd, J=13.0, 4.4 Hz, 1H), 4.42 (d, J=12.6 Hz, 1H), 4.22 (s, 1H), 4.13 (d, J=10.4 Hz, 1H), 3.93 (d, J=10.3 Hz, 1H), 3.15-3.06 (m, 2H), 3.06-2.91 (m, 3H), 2.83 (d, J=6.7 Hz, 1H), 2.44-2.32 (m, 8H), 2.23-2.10 (m, 1H), 2.08-1.92 (m, 2H), 1.88-1.72 (m, 4H), 1.65-1.35 (m, 4H).

### Example 47: Compound 47: 6-((2R,5aS,6R,9R)-3-Chloro-1-fluoro-13-(((2R,7aS)-2-fluorotetrahydro-1H-pyrrolizin-7a(5H)-yl)methoxy)-15-(oxetan-3-ylmethyl)-5a,6,7,8,9,10-hexahydro-5H-9,6-(epiminomethano)azepino[2′,1′:3,4][1,4]oxazepino[5,6,7-de]quinazolin-2-yl)-4-methyl-5-(trifluoromethyl)pyridin-2-amine

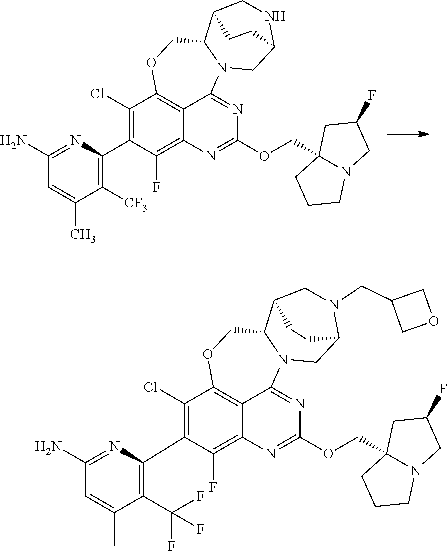

A solution of 6-((5aS,6R,9R)-3-Chloro-1-fluoro-13-(((2R,7aS)-2-fluorotetrahydro-1H-pyrrolizin-7a(5H)-yl)methoxy)-5a,6,7,8,9,10-hexahydro-5H-9,6-(epiminomethano)azepino[2′,1′:3,4][1,4]oxazepino[5,6,7-de]quinazolin-2-yl)-4-methyl-5-(trifluoromethyl)pyridin-2-amine (60.0 mg, 0.0900 mmol, Example 45, step 3, isomer 2), oxetane-3-carbaldehyde (10.1 mg, 0.120 mmol), AcOH (7.5 mg, 0.0900 mmol) in methyl alcohol (1.5 mL) was stirred at room temperature for 1 h. Then NaBH₃CN (17.1 mg, 0.270 mmol) was added and stirred at room temperature for 1 hour. The reaction system was concentrated under vacuum. The crude product was purified by Prep-HPLC with the following conditions: (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 36% B to 61% B in 9 min; Wavelength: 254/220 nm; R_{T} (min): 8.85) to yield 7.4 mg as a white solid. LC-MS: (ESI, m/z): [M+H]⁺=736. ¹H NMR (300 MHz, DMSO-d6, ppm) δ 6.84 (s, 2H), 6.48 (s, 1H), 5.40-5.15 (m, 1H), 4.93 (dd, J=13.6, 5.3 Hz, 1H), 4.72-4.55 (m, 3H), 4.41 (d, J=12.7 Hz, 1H), 4.30 (t, J=6.0 Hz, 2H), 4.18 (s, 1H), 4.12 (d, J=10.3 Hz, 1H), 3.93 (d, J=10.3 Hz, 1H), 3.28-3.06 (m, 4H), 3.00 (s, 3H), 2.93 (d, J=7.6 Hz, 2H), 2.90-2.78 (m, 1H), 2.56 (s, 1H), 2.39-2.32 (m, 4H), 2.15 (d, J=4.6 Hz, 1H), 2.09-1.90 (m, 2H), 1.88-1.72 (m, 4H), 1.60-1.34 (m, 3H).

### Example 48: Compound 48: 4-((2R,5aS,6R,9R)-2-(6-Amino-4-methyl-3-(trifluoromethyl)pyridin-2-yl)-3-chloro-1-fluoro-13-(((2R,7aS)-2-fluorotetrahydro-1H-pyrrolizin-7a(5H)-yl)methoxy)-5a,6,7,8,9,10-hexahydro-5H-9,6-(epiminomethano)azepino[2,1′:3,4][1,4]oxazepino[5,6,7-de]quinazolin-15-yl)butanenitrile

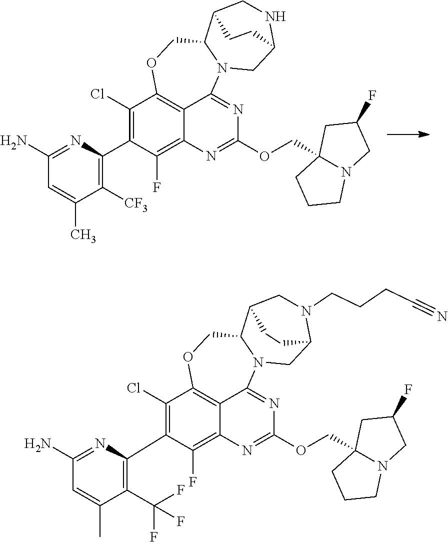

A solution of 6-((5aS,6R,9R)-3-Chloro-1-fluoro-13-(((2R,7aS)-2-fluorotetrahydro-1H-pyrrolizin-7a(5H)-yl)methoxy)-5a,6,7,8,9,10-hexahydro-5H-9,6-(epiminomethano)azepino[2′,1′:3,4][1,4]oxazepino[5,6,7-de]quinazolin-2-yl)-4-methyl-5-(trifluoromethyl)pyridin-2-amine (70.0 mg, 0.110 mmol, Example 45, step 3, isomer 2), 4-bromobutyronitrile (31.0 mg, 0.210 mmol) and DIPEA (67.9 mg, 0.530 mmol) in acetonitrile (1.5 mL) was stirred at 60° C. overnight. The crude product was purified by Prep-HPLC with the following conditions: (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 40% B to 65% B in 9 min, Wavelength: 254/220 nm; Rr (min): 8.55) to yield 9.0 mg of the title compound as a white solid. LC-MS: (ESI, m/z): [M+H]⁺=733. ¹H NMR (300 MHz, DMSO-d6, ppm) δ 6.81 (s, 2H), 6.46 (s, 1H), 5.40-5.13 (m, 1H), 4.92 (dd, J=13.6, 5.2 Hz, 1H), 4.61 (dd, J=13.0, 4.1 Hz, 1H), 4.40 (d, J=12.7 Hz, 1H), 4.23 (s, 1H), 4.10 (d, J=10.3 Hz, 1H), 3.91 (d, J=10.3 Hz, 1H), 3.33 (s, 1H), 3.13-2.95 (m, 5H), 2.81 (q, J=8.8, 8.2 Hz, 1H), 2.67 (t, J=6.4 Hz, 2H), 2.55 (t, J=7.0 Hz, 3H), 2.38-2.30 (m, 4H), 2.17-2.03 (m, 1H), 1.99 (d, J=13.4 Hz, 2H), 1.74 (dt, J=13.9, 7.7 Hz, 6H), 1.60-1.30 (m, 3H).

### Example 49: Compound 49: 6-((2R,5aS,6R,9R)-3-Chloro-1-fluoro-13-(((S,Z)-2-(fluoromethylene)tetrahydro-1H-pyrrolizin-7a(5H)-yl)methoxy)-5a,6,7,8,9,10-hexahydro-5H-9,6-(epiminomethano)azepino[2′,1′:3,4][1,4]oxazepino[5,6,7-de]quinazolin-2-yl)-4-methyl-5-(trifluoromethyl)pyridin-2-amine

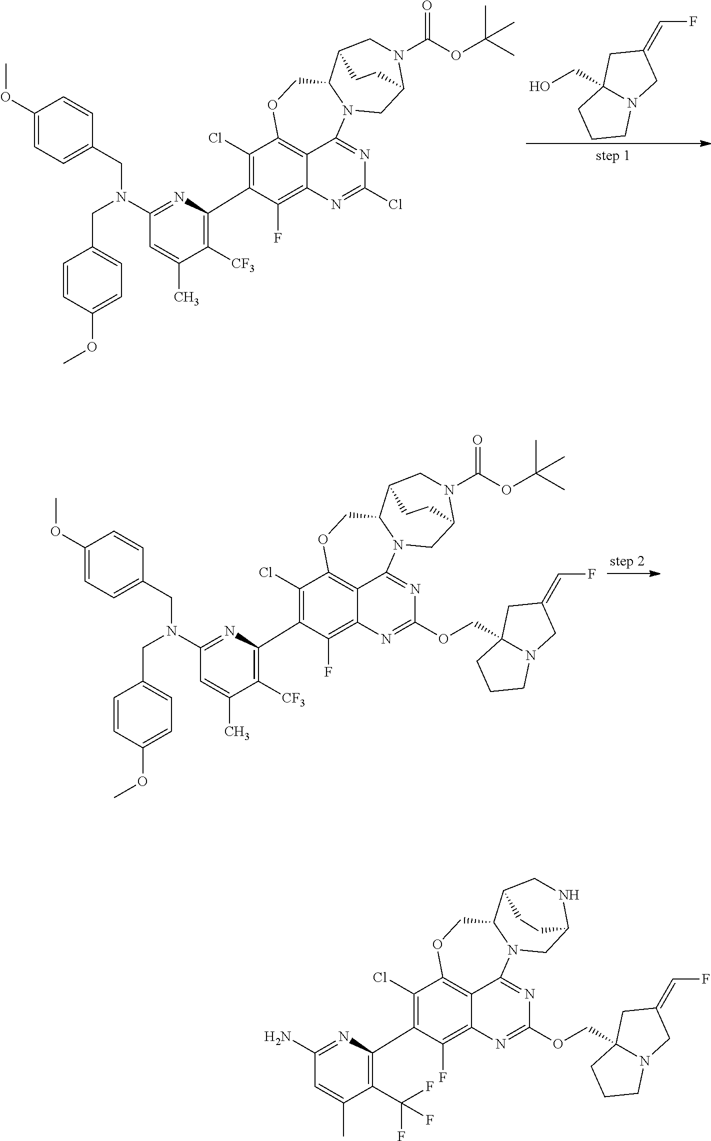

### Step 1: tert-Butyl (2R,5aS,6R,9R)-2-(6-(bis(4-methoxybenzyl)amino)-4-methyl-3-(trifluoromethyl)pyridin-2-yl)-3-chloro-1-fluoro-13-(((S,Z)-2-(fluoromethylene)tetrahydro-1H-pyrrolizin-7a(5H)-yl)methoxy)-5a,6,7,8,9,10-hexahydro-5H-9,6-(epiminomethano)azepino[2′,1′:3,4][1,4]oxazepino[5,6,7-de]quinazoline-15-carboxylate

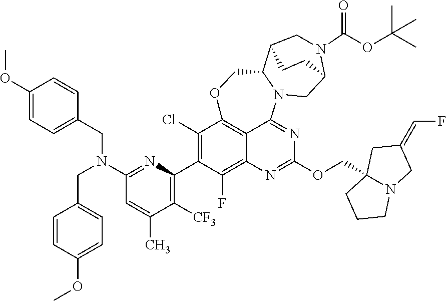

Under nitrogen, to a solution of [rac-(6Z,8S)-6-(fluoromethylene)-2,3,5,7-tetrahydro-1H-pyrrolizin-8-yl]methanol (42.6 mg, 0.250 mmol, intermediate 39) in THF (3 mL) was added NaH (24.9 mg, 0.620 mmol, 60% in mineral oil) at 0° C. The resulting solution was stirred at room temperature for 30 min. Then tert-butyl rac-(1R,2S,16R)-7-[6-[bis[(4-methoxyphenyl)methyl]amino]-4-methyl-3-(trifluoromethyl)-2-pyridyl]-6,11-dichloro-8-fluoro-4-oxa-10,12,14,17-tetrazapentacyclo[14.2.2.15,9.02,14.013,21]henicosa-5 (21),6,8,10,12-pentaene-17-carboxylate (110 mg, 0.120 mmol, Example 45, step 2, atropisomer 2) was added. The solution was stirred at 25° C. for 2 hours. The resulting mixture was extracted with EtOAc. The combined organic layers were dried over anhydrous Na₂SO₄ and concentrated under reduced pressure. The crude product was purified by reverse phase chromatography (gradient: 0-60% acetonitrile in water (0.1% NH₄HCO₃)) to yield 95 mg (74.9% yield) of the title compound as a yellow solid. LC-MS: (ESI, m/z): [M+H]⁺=1018.

### Step 2: 6-((2R,5aS,6R,9R)-3-Chloro-1-fluoro-13-(((S,Z)-2-(fluoromethylene)tetrahydro-1H-pyrrolizin-7a(5H)-yl)methoxy)-5a,6,7,8,9,10-hexahydro-5H-9,6-(epiminomethano)azepino[2′,1′:3,4][1,4]oxazepino[5,6,7-de]quinazolin-2-yl)-4-methyl-5-(trifluoromethyl)pyridin-2-amine

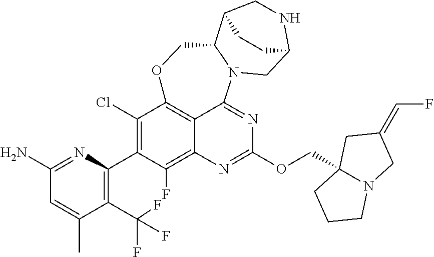

A solution of tert-butyl rac-(1R,2S,16R)-7-[6-[bis[(4-methoxyphenyl)methyl]amino]-4-methyl-3-(trifluoromethyl)-2-pyridyl]-6-chloro-8-fluoro-11-[[rac-(6Z,8S)-6-(fluoromethylene)-2,3,5,7-tetrahydro-1H-pyrrolizin-8-yl]methoxy]-4-oxa-10,12,14,17-tetrazapentacyclo[14.2.2.15,9.02,14.013,21]henicosa-5 (21),6,8,10,12-pentaene-17-carboxylate (85.0 mg, 0.080 mmol) in TFA (2 mL) was stirred at 50 degrees C. for 4 hours. Solvent was evaporated under vacuum. Adjust the pH to 10 with NaHCO₃ and then extracted with DCM. The organic layer was dried over Na₂SO₄ and concentrated under vacuum to yield 54 mg (crude) of the title compound as a white crude solid. A small fraction of the crude project was purified by prep-HPLC to afford 2 mg of the title compound. LC-MS: (ESI, m/z): [M+H]⁺=678. ¹H NMR (300 MHz, DMSO-d6, ppm) δ 6.87 (d, J=17.0 Hz, 2H), 6.65-6.60 (m, 1H), 6.47 (s, 1H), 4.48 (t, J=5.9 Hz, 1H), 4.41 (d, J=12.7 Hz, 1H), 4.20 (s, 1H), 4.06 (d, J=10.5 Hz, 1H), 3.99 (d, J=10.5 Hz, 1H), 3.78 (d, J=14.6 Hz, 1H), 3.33-3.21 (m, 2H), 3.11-2.80 (m, 3H), 2.75 (m, 2H), 2.54 (d, J=15.0 Hz, 3H), 2.41-2.25 (m, 5H), 2.01-1.98 (m, 2H), 1.82-1.42 (m, 5H).

### Example 50: Compound 50: 6-((2R,5aS,6R,9R)-3-Chloro-1-fluoro-13-(((S,Z)-2-(fluoromethylene)tetrahydro-1H-pyrrolizin-7a(5H)-yl)methoxy)-15-(3-fluoropropyl)-5a,6,7,8,9,10-hexahydro-5H-9,6-(epiminomethano)azepino[2′,1′:3,4][1,4]oxazepino[5,6,7-de]quinazolin-2-yl)-4-methyl-5-(trifluoromethyl)pyridin-2-amine

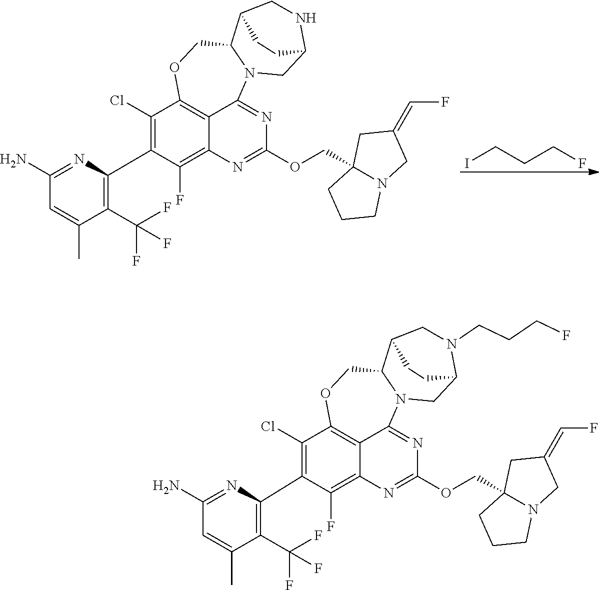

A solution of 4-methyl-6-[rac-(1R,2S,16R)-6-chloro-8-fluoro-11-[[rac-(6Z,8S)-6-(fluoromethylene)-2,3,5,7-tetrahydro-1H-pyrrolizin-8-yl]methoxy]-4-oxa-10,12,14,17-tetrazapentacyclo[14.2.2.15,9.02,14.013,21]henicosa-5 (21),6,8,10,12-pentaen-7-yl]-5-(trifluoromethyl)pyridin-2-amine (60.0 mg, 0.090 mmol, Example 49), 1-fluoro-3-iodopropane (33.3 mg, 0.180 mmol) and DIPEA (57.1 mg, 0.440 mmol) in acetonitrile (2 mL) was stirred at 50 degrees C. for 18 hours. The crude product was purified by reverse phase chromatography (gradient: 0-60% acetonitrile in water (0.1% NH₄HCO₃)) to yield 33.0 mg (50.5% yield) of title compound as a white solid. LC-MS: (ESI, m/z): [M+H]⁺=738. ¹H NMR (400 MHz, DMSO-d6, ppm) δ 6.84 (d, J=17.0 Hz, 2H), 6.68-6.62 (m, 1H), 6.47 (s, 1H), 4.94 (m, 1H), 4.61 (m, 2H), 4.48 (t, J=5.9 Hz, 1H), 4.42 (d, J=12.7 Hz, 1H), 4.22 (s, 1H), 4.08 (d, J=10.5 Hz, 1H), 3.95 (d, J=10.5 Hz, 1H), 3.70 (d, J=14.6 Hz, 1H), 3.31-3.23 (m, 2H), 3.12-2.94 (m, 3H), 2.70 (m, 2H), 2.56 (d, J=15.0 Hz, 3H), 2.42-2.28 (m, 5H), 1.94 (m, 1H), 1.82 (m, 5H), 1.73-1.62 (m, 1H), 1.61-1.48 (m, 2H), 1.42 (d, J=13.6 Hz, 1H)

### Example 51: Compound 51A & 51B: 6-((5S,5aS,6R,9R)-3-Chloro-1-fluoro-13-(((2R,7aS)-2-fluorotetrahydro-1H-pyrrolizin-7a(5H)-yl)methoxy)-5-methyl-5a,6,7,8,9,10-hexahydro-5H-9,6-(epiminomethano)azepino[2′,1′:3,4][1,4]oxazepino[5,6,7-de]quinazolin-2-yl)-4-methyl-5-(trifluoromethyl)pyridin-2-amine

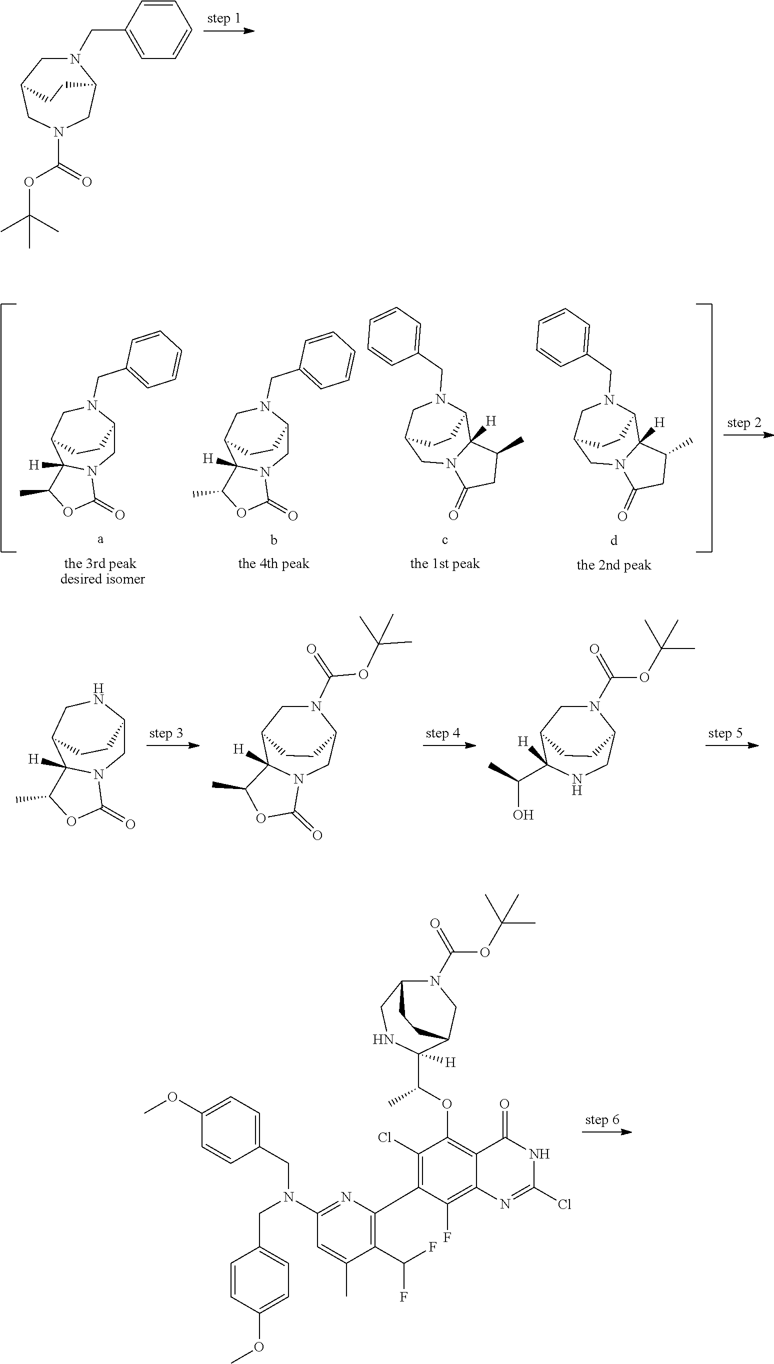

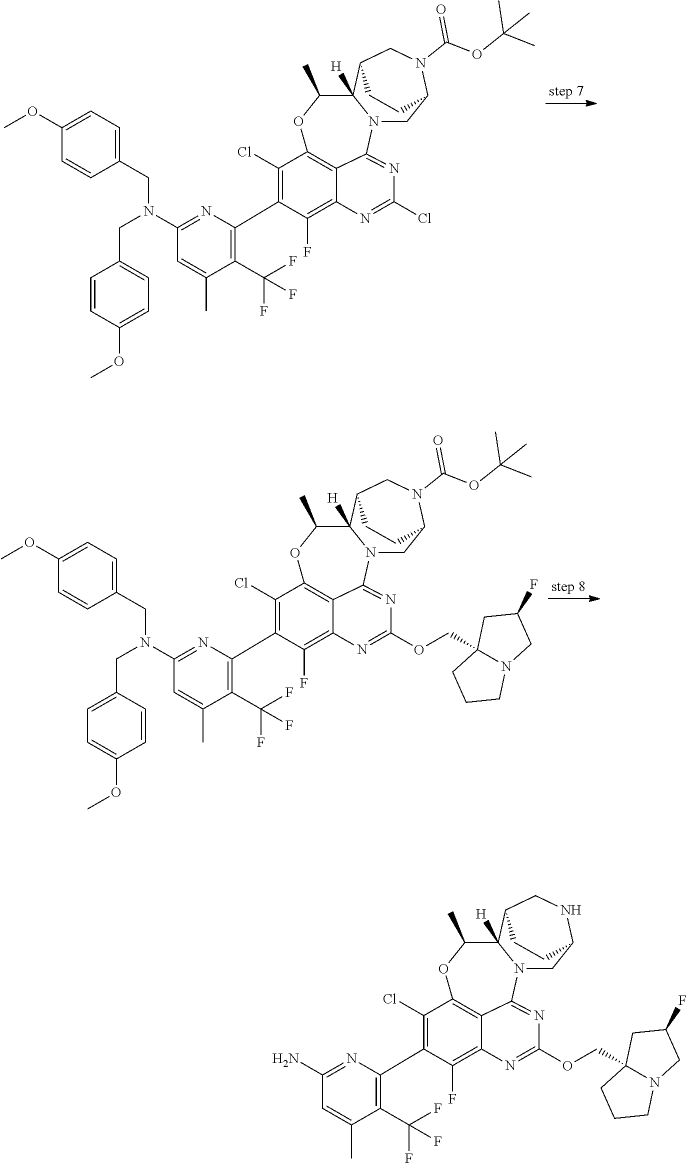

### Step 1: (1S,6R,9R,9aS)-11-Benzyl-1-methylhexahydro-1H,3H-6,9-(epiminomethano)oxazolo [3,4-a]azepin-3-one

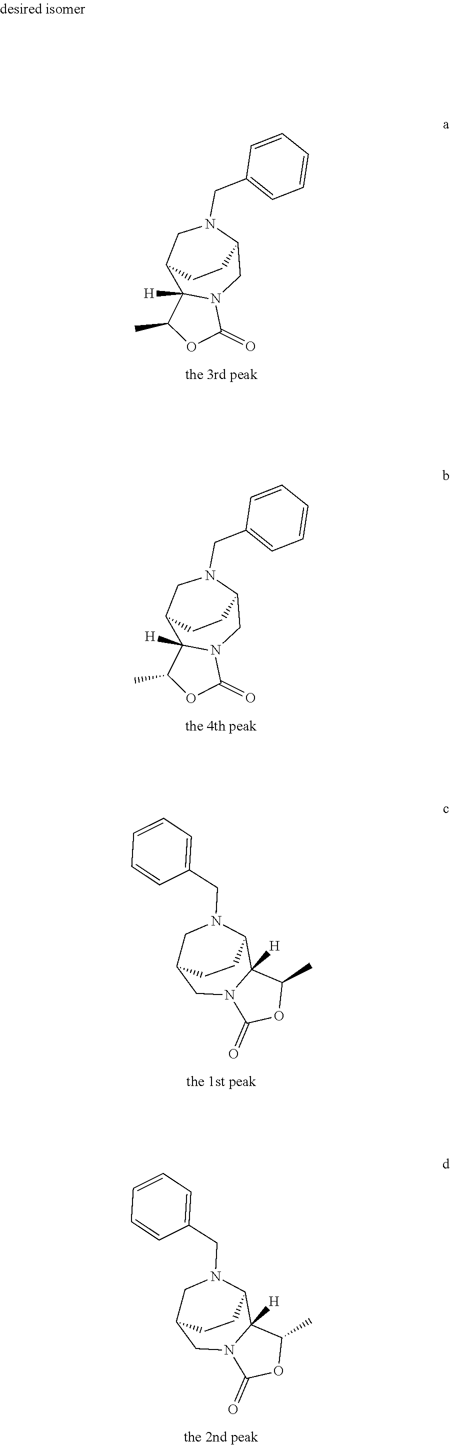 [SMILES: C[C@@H]1OC(=O)N2C[C@H]3CC[C@H](CN3Cc3ccccc3)[C@@H]12 ; C[C@H]1OC(=O)N2C[C@H]3CC[C@H](CN3Cc3ccccc3)[C@@H]12 ; C[C@H]1OC(=O)N2C[C@@H]3CC[C@H]([C@H]12)N(Cc1ccccc1)C3 ; C[C@@H]1OC(=O)N2C[C@@H]3CC[C@H]([C@H]12)N(Cc1ccccc1)C3]

Under nitrogen, to a solution of tert-butyl (1R,5R)-6-benzyl-3,6-diazabicyclo[3.2.2]nonane-3-carboxylate (10.0 g, 31.6 mmol) and TMEDA (7.35 g, 63.6 mmol) in diethyl ether (150 mL) was added s-BuLi (48.5 mL, 63.1 mmol, 1.3 M in cyclohexane) at −78° C. The resulting solution was stirred for 1.5 h at −55° C. The reaction system was cooled to −78° C. and acetaldehyde (4.4 mL, 78.5 mmol) in diethyl ether (5 mL) was added at this temperature. The reaction system was warmed naturally to room temperature and stirred overnight. The mixture was quenched with aq. NH₄Cl, extracted with EtOAc. The combined organic layers were dried over anhydrous Na₂SO₄ and concentrated under vacuum. The residue was purified by flash chromatography on silica gel (gradient: 0-30% EtOAc/petroleum ether) to yield the title compound 4.4 g (mixture 4 isomers). The mixture was separated by PREP_SFC (Column: CHIRALPAK IG, 5*25 cm, 10 μm; Mobile Phase A: CO₂, Mobile Phase B: MEOH (0.1% 2M NH₃-MEOH); Flow rate: 200 mL/min; Gradient: isocratic 60% B; Column Temperature (° C.): 35; Back Pressure (bar): 100; Wave Length: 220 nm; RT1 (min): 5.73; RT2 (min): 6.9; Sample Solvent: MeCOH—-Preparative; Injection Volume: 4 mL; Number Of Runs: 23) to yield mixture of compound c and d 3.2 g (35.3% yield) (mixture of the first peak and second peak on chiral SFC) and compound a 490 mg (5.4% yield) (the third peak, desired isomer) and compound b140 mg (1.5% yield) (the fourth peak) as yellow solid. LC-MS: (ESI, m/z): [M+H]⁺=287.

### Step 2: (1S,6R,9R,9aS)-1-Methylhexahydro-1H,3H-6,9-(epiminomethano)oxazolo[3,4-a]azepin-3-one

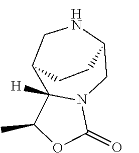

Under hydrogen, to a solution of (1S,6R,9R,9aS)-11-benzyl-1-methylhexahydro-1H,3H-6,9-(epiminomethano)oxazolo [3,4-a]azepin-3-one (490 mg, 1.71 mmol) (from the third peak of the first step) in methyl alcohol (10 mL) was added Pd/C (250 mg, 10%) at room temperature. The resulting solution was stirred for 1 h at room temperature. The catalyst was filtered off. The filtrate was concentrated under vacuum to yield the title compound 500 mg (crude) as a yellow oil, which was used for next step without further purification. LC-MS: (ESI, m/z): [M+H]⁺=197.

### Step 3: tert-Butyl (1 S,6R,9R,9aS)-1-Methyl-3-oxohexahydro-1H,3H-6,9-(epiminomethano) oxazolo [3,4-a]azepine-11-carboxylate

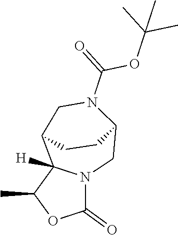 [SMILES: C[C@@H]1OC(=O)N2C[C@H]3CC[C@H](CN3C(=O)OC(C)(C)C)[C@@H]12]

A solution of (1S,6R,9R,9aS)-1-methylhexahydro-1H,3H-6,9-(epiminomethano) oxazolo[3,4-a]azepin-3-one (500 mg, crude), Boc₂O (834 mg, 3.83 mmol) and DIPEA (987 mg, 7.65 mmol) in dichloromethane (10 mL) was stirred for 30 min at room temperature. Concentrated under vacuum. The residue was purified by pre-packed C18 column (gradient: 0-100% ACN in water (0.05% NH₄HCO₃)) to yield the title compound 332 mg (44% yield) as a white solid. LC-MS: (ESI, m/z): [M+H]⁺=296.

### Step 4 tert-Butyl (1R,2S,5R)-2-((S)-1-hydroxyethyl)-3,6-diazabicyclo[3.2.2]nonane-6-carboxylate

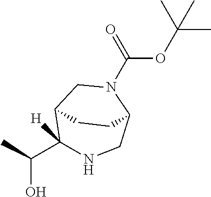 [SMILES: C[C@H](O)[C@H]1NC[C@H]2CC[C@@H]1CN2C(=O)OC(C)(C)C]

To a solution of tert-butyl (1 S,6R,9R,9aS)-1-Methyl-3-oxohexahydro-1H,3H-6,9-(epiminomethano) oxazolo [3,4-a]azepine-11-carboxylate (332 mg, 1.12 mmol) in ethanol (5 mL) and water (1 mL) was added NaOH (448 mg, 11.2 mmol). The resulting solution was stirred for 1 h at 80° C. The reaction mixture was partitioned between water and DCM. The combined organic layers were dried over anhydrous Na₂SO₄ and concentrated under vacuum to yield the title compound 301 mg (crude) as yellow solid which was used for next step without further purification. LC-MS: (ESI, m/z): [M+H]⁺=271.

### Step 5: tert-Butyl (1R,2S,5R)-2-((1S)-1-((7-(6-(bis(4-methoxybenzyl)amino)-4-methyl-3-(trifluoromethyl)pyridin-2-yl)-2,6-dichloro-8-fluoro-4-oxo-3,4-dihydroquinazolin-5-yl)oxy)ethyl)-3,6-diazabicyclo[3.2.2]nonane-6-carboxylate

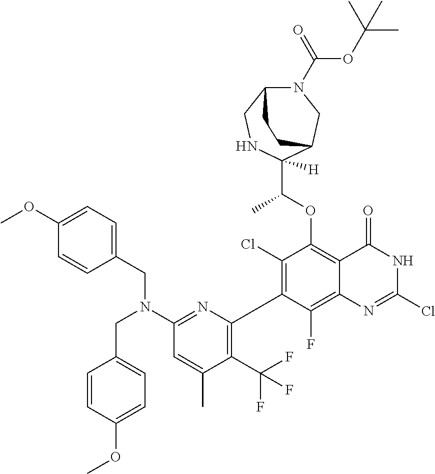 [SMILES: COc1ccc(CN(Cc2ccc(OC)cc2)c2cc(C)c(C(F)(F)F)c(-c3c(Cl)c(O[C@H](C)[C@H]4NC[C@H]5CC[C@@H]4CN5C(=O)OC(C)(C)C)c4c(=O)[nH]c(Cl)nc4c3F)n2)cc1]

Under nitrogen, To a solution of tert-butyl (1R,2S,5R)-2-((S)-1-hydroxyethyl)-3,6-diazabicyclo[3.2.2]nonane-6-carboxylate (276 mg, 1.02 mmol) in dimethyl sulfoxide (6 mL) was added NaHMDS (2.5 mL, 5 mmol, 2 M in THF) at room temperature. The reaction system was stirred for 0.5 h at room temperature and then a solution of 7-(6-(bis(4-methoxybenzyl)amino)-4-methyl-3-(trifluoromethyl)pyridin-2-yl)-2,6-dichloro-5,8-difluoroquinazolin-4 (3H)-one (678 mg, 1.02 mmol, intermediate 2) in dimethyl sulfoxide (6 mL) was added. The resulting solution was stirred for 1 h at room temperature. The mixture was quenched with aq. NH₄Cl and extracted with EtOAc. The combined organic layers were dried over anhydrous Na₂SO₄ and concentrated under vacuum. The residue was purified by flash chromatography on silica gel (gradient: 0-10% MeCOH/DCM) to yield the title compound 380 mg (40.7% yield) as a yellow oil. LC-MS: (ESI, m/z): [M+H]⁺=915.

### Step 6: tert-Butyl (5S,5aS,6R,9R)-2-(6-(bis(4-methoxybenzyl)amino)-4-methyl-3-(trifluoromethyl)pyridin-2-yl)-3,13-dichloro-1-fluoro-5-methyl-5a,6,7,8,9,10-hexahydro-5H-9,6-(epiminomethano)azepino[2′,1′:3,4][1,4]oxazepino[5,6,7-de]quinazoline-15-carboxylate

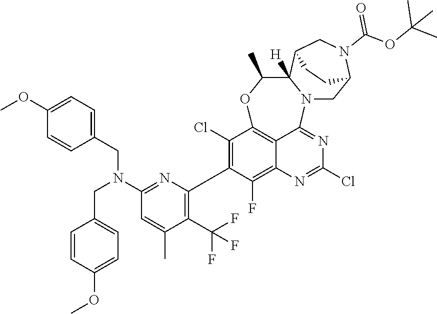 [SMILES: COc1ccc(CN(Cc2ccc(OC)cc2)c2cc(C)c(C(F)(F)F)c(-c3c(Cl)c4c5c(nc(Cl)nc5c3F)N3C[C@H]5CC[C@H](CN5C(=O)OC(C)(C)C)[C@H]3[C@H](C)O4)n2)cc1]

A solution of tert-butyl (1R,2S,5R)-2-((1S)-1-((7-(6-(bis(4-methoxybenzyl)amino)-4-methyl-3-(trifluoromethyl)pyridin-2-yl)-2,6-dichloro-8-fluoro-4-oxo-3,4-dihydroquinazolin-5-yl)oxy)ethyl)-3,6-diazabicyclo[3.2.2]nonane-6-carboxylate (360 mg, 0.390 mmol), BOPCl (400 mg, 1.58 mmol) and DIPEA (762 mg, 5.91 mmol) in dichloromethane (5 mL) was stirred overnight at room temperature. Concentrated under vacuum. The residue was purified by flash chromatography on silica gel (gradient: 0-50% EtOAc/petroleum ether) to yield the title compound 170 mg (48.2% yield) (mixture of 2 atropisomers) as a yellow solid. LC-MS: (ESI, m/z): [M+H]⁺=897.

### Step 7: tert-Butyl (5S,5aS,6R,9R)-2-(6-(bis(4-methoxybenzyl)amino)-4-methyl-3-(trifluoromethyl)pyridin-2-yl)-3-chloro-1-fluoro-13-(((2R,7aS)-2-fluorotetrahydro-1H-pyrrolizin-7a(5H)-yl)methoxy)-5-methyl-5a,6,7,8,9,10-hexahydro-5H-9,6-(epiminomethano)azepino[2′,1′:3,4][1,4]oxazepino[5,6,7-de]quinazoline-15-carboxylate

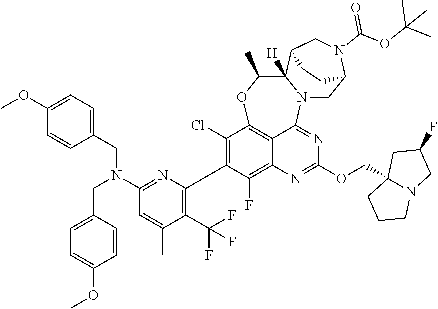 [SMILES: COc1ccc(CN(Cc2ccc(OC)cc2)c2cc(C)c(C(F)(F)F)c(-c3c(Cl)c4c5c(nc(OC[C@@]67CCCN6C[C@H](F)C7)nc5c3F)N3C[C@H]5CC[C@H](CN5C(=O)OC(C)(C)C)[C@H]3[C@H](C)O4)n2)cc1]

Under nitrogen, to a solution of ((2R,7aS)-2-fluorotetrahydro-1H-pyrrolizin-7a(5H)-yl)methanol (33.9 mg, 0.210 mmol, intermediate 4) in tetrahydrofuran (3 mL) was added NaH (17.9 mg, 0.450 mmol, 60% in mineral oil) at 0° C. The resulting solution was stirred for 30 min at 0° C. Then tert-butyl (5S,5aS,6R,9R)-2-(6-(bis(4-methoxybenzyl)amino)-4-methyl-3-(trifluoromethyl)pyridin-2-yl)-3,13-dichloro-1-fluoro-5-methyl-5a,6,7,8,9,10-hexahydro-5H-9,6-(epiminomethano)azepino[2′,1′:3,4][1,4]oxazepino[5,6,7-de]quinazoline-15-carboxylate (160 mg, 0.180 mmol) was added at 0° C. The solution was stirred for 2 h at 40° C. The reaction was quenched with water and concentrated under vacuum. The residue was purified by flash chromatography on silica gel (gradient: 0-10% MeCOH/DCM) to yield the title compound 126 mg (69.3% yield) as a white solid. LC-MS: (ESI, m/z): [M+H]⁺=1020.

### Step 8: 6-((5S,5aS,6R,9R)-3-Chloro-1-fluoro-13-(((2R,7aS)-2-fluorotetrahydro-1H-pyrrolizin-7a(5H)-yl)methoxy)-5-methyl-5a,6,7,8,9,10-hexahydro-5H-9,6-(epiminomethano)azepino[2′,1′:3,4][1,4]oxazepino[5,6,7-de]quinazolin-2-yl)-4-methyl-5-(trifluoromethyl)pyridin-2-amine

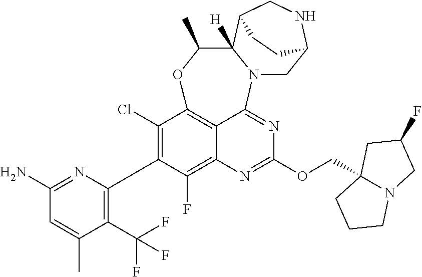 [SMILES: Cc1cc(N)nc(-c2c(Cl)c3c4c(nc(OC[C@@]56CCCN5C[C@H](F)C6)nc4c2F)N2C[C@H]4CC[C@H](CN4)[C@H]2[C@H](C)O3)c1C(F)(F)F]

A solution of tert-butyl (5S,5aS,6R,9R)-2-(6-(bis(4-methoxybenzyl)amino)-4-methyl-3-(trifluoromethyl)pyridin-2-yl)-3-chloro-1-fluoro-13-(((2R,7aS)-2-fluorotetrahydro-1H-pyrrolizin-7a(5H)-yl)methoxy)-5-methyl-5a,6,7,8,9,10-hexahydro-5H-9,6-(epiminomethano)azepino[2′,1′:3,4][1,4]oxazepino[5,6,7-de]quinazoline-15-carboxylate (120 mg, 0.120 mmol) in 2,2,2-trifluoroacetic acid (2 mL) was stirred for 4 h at 50° C. Concentrated under vacuum. The residue was purified by pre-packed C18 column (gradient: 0-100% MeCOH in water (0.05% NH₄HCO₃)) to yield the title compound 70 mg (87.5% yield) (mixture of 2 atropisomers) as a white solid. LC-MS: (ESI, m/z): [M+H]⁺=680. ¹H NMR (300 MHz, DMSO-d₆) δ 6.84 (d, J=13.1 Hz, 2H), 6.48 (s, 1H), 5.40-5.16 (m, 2H), 4.76-4.58 (m, 1H), 4.18-3.91 (m, 3H), 3.30-3.19 (m, 2H), 3.14-3.05 (m, 2H), 3.03-2.91 (m, 3H), 2.86-2.78 (m, 1H), 2.37-2.35 (m, 3H), 2.25-2.06 (m, 2H), 2.06-1.97 (m, 2H), 1.88-1.67 (m, 5H), 1.60-1.51 (m, 2H), 1.34-1.27 (m, 4H).

### Example 51: Compound 51A & 51B: 6-((5S,5aS,6R,9R)-3-Chloro-1-fluoro-13-(((2R,7aS)-2-fluorotetrahydro-1H-pyrrolizin-7a(5H)-yl)methoxy)-5-methyl-15-(oxetan-3-ylmethyl)-5a,6,7,8,9,10-hexahydro-5H-9,6-(epiminomethano)azepino[2′,1′:3,4][1,4]oxazepino[5,6,7-de]quinazolin-2-yl)-4-methyl-5-(trifluoromethyl)pyridin-2-amine

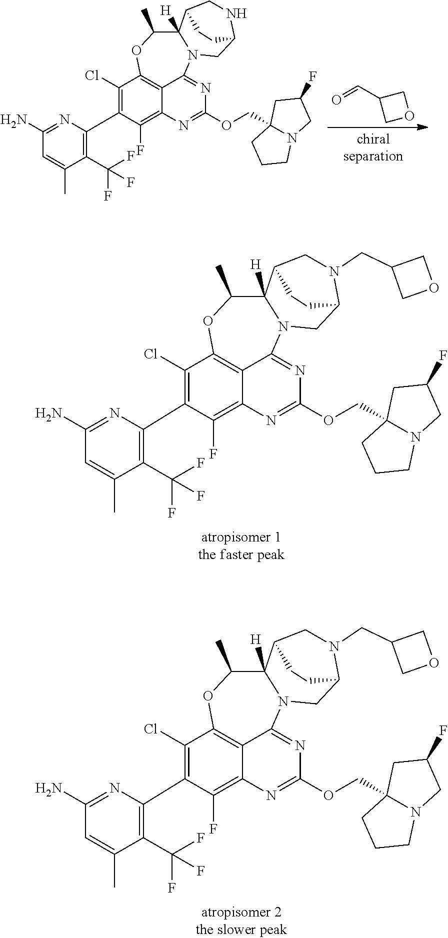

A solution of 6-((5S,5aS,6R,9R)-3-chloro-1-fluoro-13-(((2R,7aS)-2-fluorotetrahydro-1H-pyrrolizin-7a(5H)-yl)methoxy)-5-methyl-5a,6,7,8,9,10-hexahydro-5H-9,6-(epiminomethano)azepino[2′,1′:3,4][1,4]oxazepino[5,6,7-de]quinazolin-2-yl)-4-methyl-5-(trifluoromethyl)pyridin-2-amine (60.0 mg, 0.0900 mmol), oxetane-3-carbaldehyde (11.4 mg, 0.130 mmol) and AcOH (8.00 mg, 0.130 mmol) in methyl alcohol (2 mL) was stirred for 1 h at room temperature. Then NaBH₃CN (8.30 mg, 0.130 mmol) was added at room temperature and stirred additional 1 h. Concentrated under vacuum. The residue was purified by pre-packed C18 column (gradient: 0-100% MeCOH in water (0.05% NH₄HCO₃)) to yield 30 mg title compound (mixture of 2 atropisomers). The mixture was separated by PREP_CHIRAL_HPLC (Column: CHIRAL ART Cellulose-SC, 2*25 cm, 5 μm; Mobile Phase A: Hex:DCM=3:1 (0.5% 2M NH3-MeOH)—HPLC, Mobile Phase B: EtOH—HPLC; Flow rate: 20 mL/min; Gradient: 20% B to 20% B in 12 min; Wave Length: 220/254 nm; RT1 (min): 6.398; RT2 (min): 9.95; Sample Solvent: EtOH; Injection Volume: 1.5 mL; Number Of Runs: 2) to yield 5.9 mg of Compound 51 (the faster peak) and 4.3 mg of Compound 51 (the slower peak) as white solids. LC-MS: (ESI, m/z): [M+H]⁺=750.

Compound 51: ¹H NMR (300 MHz, DMSO-d₆) δ 6.85 (s, 2H), 6.46 (s, 1H), 5.27 (d, J=54.4 Hz, 1H), 5.10-5.02 (m, 1H), 4.75-4.57 (m, 3H), 4.28-4.23 (m, 2H), 4.10 (d, J=10.4 Hz, 1H), 4.05-3.84 (m, 2H), 3.41 (d, J=13.6 Hz, 1H), 3.18-2.96 (m, 5H), 2.90 (d, J=7.4 Hz, 3H), 2.82 (d, J=7.4 Hz, 1H), 2.54 (d, J=10.1 Hz, 1H), 2.34 (d, J=2.1 Hz, 3H), 2.20 (s, 1H), 2.17-2.10 (m, 1H), 2.09-1.95 (m, 2H), 1.87-1.71 (m, 4H), 1.62-1.48 (m, 2H), 1.37-1.29 (d, J=6.9 Hz, 1H), 1.26 (d, J=6.5 Hz, 3H).

Compound 51: ¹H NMR (300 MHz, DMSO-d₆) δ 6.81 (s, 2H), 6.46 (s, 1H), 5.40-5.12 (m, 1H), 5.06-4.99 (m, 1H), 4.69-4.50 (m, 3H), 4.29-4.23 (m, 2H), 4.11-3.92 (m, 3H), 3.37 (d, J=13.6 Hz, 1H), 3.14-3.05 (m, 4H), 2.99 (s, 1H), 2.94-2.75 (m, 4H), 2.62-2.57 (m, 1H), 2.34 (d, J=2.3 Hz, 3H), 2.23-2.09 (m, 2H), 2.09-1.90 (m, 2H), 1.87-1.58 (m, 5H), 1.56-1.08 (m, 5H).

TABLE 4
NMR

| Cmpd | |
|---|---|
| No | ¹H NMR |
| 1A | ¹H NMR (400 MHz, DMSO-d₆, ppm) δ 6.81 (s, 2H), 6.47 (s, 1H), 4.90 (s, 2H), 4.78-4.71 (m, |
| | 1H), 4.59-4.51 (m, 1H), 4.39-4.32 (m, 1H), 4.03 (d, J = 10.8 Hz, 1H), 4.00-3.95 (m, 1H), |
| | 3.91 (d, J = 10.4 Hz, 1H), 3.59-3.50 (m, 2H), 3.49-3.42 (m, 1H), 3.23-3.12 (m, 1H), |
| | 3.10-2.94 (m, 2H), 2.62-2.56 (m, 2H), 2.55-2.53 (m, 1H), 2.36 (s, 3H), 2.00-1.91 (m, 1H), |
| | 1.91-1.82 (m, 1H), 1.82-1.74 (m, 2H), 1.71-1.61 (m, 3H), 1.60-1.50 (m, 1H). |
| 1B | ¹H NMR (300 MHz, DMSO-d₆, ppm) δ 6.81 (s, 2H), 6.47 (s, 1H), 4.93-4.80 (m, 3H), 4.63- |
| | 4.53 (m, 1H), 4.35-4.22 (m, 1H), 4.09-3.88 (m, 3H), 3.62-3.50 (m, 2H), 3.47-3.41 (m, |
| | 1H), 3.22-3.12 (m, 1H), 3.09-2.93 (m, 2H), 2.63-2.54 (m, 2H), 2.41-2.30 (m, 4H), |
| | 2.02-1.90 (m, 1H), 1.89-1.74 (m, 2H), 1.73-1.61 (m, 4H), 1.60-1.50 (m, 1H). |
| 2 | ¹H NMR (400 MHz, DMSO-d₆, ppm) δ 6.99-6.61 (m, 3H), 6.47 (s, 1H), 4.85-4.71 (m, 1H), |
| | 4.60-4.51 (m, 1H), 4.46-4.31 (m, 1H), 4.07 (d, J = 10.4 Hz, 1H), 4.03-3.91 (m, 2H), 3.74- |
| | 3.69 (m, 1H), 3.65-3.59 (m, 1H), 3.57-3.48 (m, 1H), 3.30-3.26 (m, 1H), 3.12-2.95 (m, |
| | 2H), 2.61-2.52 (m, 2H), 2.41-2.31 (m, 4H), 1.99-1.91 (m, 1H), 1.91-1.74 (m, 3H), 1.73- |
| | 1.61 (m, 3H), 1.60-1.50 (m, 1H). |
| 3 | ¹H NMR (400 MHz, DMSO-d₆, ppm) δ 6.83 (s, 2H), 6.47 (s, 1H), 4.92 (s, 2H), 4.80-4.70 (m, |
| | 1H), 4.60-4.49 (m, 1H), 4.41-4.36 (m, 1H), 4.15-3.90 (m, 3H), 3.70-3.55 (m, 1H), 3.30- |
| | 3.20 (m, 3H), 3.19-3.10 (m, 1H), 3.03 (s, 1H), 2.76-2.58 (m, 2H), 2.45-2.31 (m, 4H), |
| | 2.26 (s, 3H), 2.08-1.48 (m, 8H). |
| 4 | ¹H NMR (400 MHz, DMSO-d₆, ppm) δ 6.83 (s, 2H), 6.48 (s, 1H), 5.09-4.77 (m, 3H), 4.74- |
| | 4.39 (m, 4H), 4.24-3.89 (m, 3H), 3.70-3.55 (m, 1H), 3.29-3.20 (m, 1H), 3.15-2.96 (m, |
| | 2H), 2.75-2.58 (m, 2H), 2.48-2.28 (m, 4H), 2.09 (s, 3H), 2.04-1.61 (m, 8H). |
| 5 | ¹H NMR (400 MHz, DMSO-d₆, ppm) δ 6.84 (s, 2H), 6.47 (s, 1H), 4.85-4.70 (m, 1H), 4.55- |
| | 4.50 (m, 1H), 4.42-4.30 (m, 1H), 4.11 (d, J = 10.8 Hz, 1H), 4.08-3.91 (m, 2H), 3.70-3.60 |
| | (m, 1H), 3.59-3.51 (m, 1H), 3.49-3.42 (m, 1H), 3.32-3.27 (m, 1H), 3.09-2.96 (m, 2H), |
| | 2.74 (s, 1H), 2.69-2.53 (m, 2H), 2.45-2.39 (m, 1H), 2.36 (s, 3H), 2.04-1.91 (m, 1H), 1.90- |
| | 1.71 (m, 4H), 1.70-1.61 (m, 2H), 1.60-1.46 (m, 1H). |
| 6A | ¹H NMR (400 MHz, DMSO-d₆, ppm) δ 6.81 (s, 2H), 6.47 (s, 1H), 4.80-4.71 (m, 1H), 4.53 (d, |
| 6B | J = 2.4 Hz, 1H), 4.42-4.29 (m, 2H), 4.15-4.09 (m, 1H), 3.99 (s, 1H), 3.65-3.39 (m, 6H), |
| | 3.19-3.02 (m, 2H), 3.01-2.81 (m, 3H), 2.36 (d, J = 1.2 Hz, 3H), 2.11-1.99 (m, 1H), 1.86- |
| | 1.69 (m, 2H), 1.68-1.47 (m, 4H), 1.35-1.21 (m, 1H). |
| | ¹H NMR (300 MHz, DMSO-d₆) d 6.82 (s, 2H), 6.47 (s, 1H), 4.81-4.71 (m, 1H), 4.59-4.51 |
| | (m, 1H), 4.45-4.32 (m, 2H), 4.11-3.96 (m, 2H), 3.67-3.39 (m, 6H), 3.20-3.03 (m, 2H), |
| | 3.03-2.91 (m, 1H), 2.90-2.83 (m, 2H), 2.37 (d, J = 2.3 Hz, 3H), 2.15-1.99 (m, 1H), 1.85- |
| | 1.72 (m, 2H), 1.71-1.48 (m, 4H), 1.39-1.21 (m, 1H). |
| 7 | ¹H NMR (300 MHz, DMSO-d₆, ppm) δ 6.80 (s, 2H), 6.45 (s, 1H), 4.83-4.65 (m, 1H), 4.62- |
| | 4.43 (m, 3H), 4.43-4.32 (m, 3H), 4.31-4.26 (m, 2H), 4.01-3.92 (m, 1H), 3.63-3.40 (m, |
| | 2H), 3.10-3.00 (m, 1H), 2.34 (s, 3H), 1.87-1.71 (m, 1H), 1.70-1.46 (m, 3H), 1.34 (s, 3H). |
| 8 | ¹H NMR (300 MHz, DMSO-d₆, ppm) δ 6.80 (s, 2H), 6.45 (s, 1H), 5.11 (s, 1H), 5.01 (s, 1H), |
| | 4.80-4.70 (m, 1H), 4.60-4.43 (m, 1H), 4.42-4.22 (m, 2H), 4.22-4.12 (m, 1H), 4.00- |
| | 3.94 (m, 1H), 3.62-3.55 (m, 1H), 3.48-3.42 (m, 1H), 3.41-3.35 (m, 1H), 3.13-2.96 (m, 2H), |
| | 2.93-2.77 (m, 1H), 2.68-2.52 (m, 3H), 2.42-2.29 (m, 4H), 2.09-2.89 (m, 1H), 1.85-1.71 (m, |
| | 1H), 1.71-1.61 (m, 2H), 1.60-1.41 (m, 1H). |
| 9 | ¹H NMR (400 MHz, DMSO-d₆, ppm) δ 6.84 (s, 2H), 6.48 (s, 1H), 4.83-4.73 (m, 1H), 4.62- |
| | 4.51 (m, 1H), 4.51-4.42 (m, 1H), 4.42-4.34 (m, 1H), 4.34-4.24 (m, 1H), 4.05-3.98 (m, |
| | 1H), 3.67-3.45 (m, 3H), 3.14-3.05 (m, 1H), 3.00-2.86 (m, 1H), 3.76-2.58 (m, 1H), 2.43- |
| | 2.32 (m, 7H), 2.29-2.12 (m, 1H), 1.87-1.78 (m, 1H), 1.72-1.64 (m, 2H), 1.64-1.51 |
| | (m, 1H). |
| 10 | ¹H NMR (300 MHz, DMSO-d₆, ppm) δ 6.84 (s, 2H), 6.47 (s, 1H), 6.21-5.81 (m, 1H), 4.85- |
| | 4.71 (m, 1H), 4.63-4.51 (m, 1H), 4.44-4.23 (m, 3H), 4.05-3.96 (m, 1H), 3.73-3.52 (m, |
| | 2H), 3.50-3.42 (m, 1H), 3.19-2.97 (m, 3H), 2.89-2.65 (m, 2H), 2.36 (s, 3H), 2.15-1.99 |
| | (m, 2H), 1.89-1.72 (m, 1H), 1.71-1.62 (m, 2H), 1.61-1.51 (m, 1H). |
| 11 | ¹H NMR (400 MHz, DMSO-d₆, ppm) δ 6.82 (s, 2H), 6.46 (s, 1H), 4.84-4.73 (m, 1H), 4.60- |
| | 4.49 (m, 1H), 4.45-4.30 (m, 1H), 4.11 (d, J = 10.4 Hz, 1H), 4.02-3.97 (m, 2H), 3.61-3.52 |
| | (m, 1H), 3.49-3.42 (m, 1H), 3.19-3.01 (m, 3H), 2.82-2.73 (m, 2H), 2.45-2.25 (m, 5H), |
| | 2.07-1.95 (m, 1H), 1.95-1.83 (m, 1H), 1.83-1.72 (m, 3H), 1.68-1.60 (m, 2H), 1.59- |
| | 1.47 (m, 1H). |
| 12 | ¹H NMR (300 MHz, DMSO-d₆, ppm) δ 6.82 (s, 2H), 6.46 (s, 1H), 4.85-4.65 (m, 1H), 4.65- |
| | 4.44 (m, 1H), 4.44-4.28 (m, 1H), 4.15-3.90 (m, 3H), 3.57 (s, 1H), 3.51-3.41 (m, 2H), |
| | 3.14-3.04 (m, 3H), 2.78-2.65 (m, 2H), 2.36-2.29 (m, 4H), 2.02-1.99 (m, 1H), 1.88- |
| | 1.41 (m, 7H). |
| 13A | ¹H NMR (400 MHz, DMSO-d₆, ppm) δ 6.81 (s, 2H), 6.47 (s, 1H), 5.02-4.75 (m, 3H), 4.69- |
| | 4.45 (m, 1H), 4.44-4.31 (m, 1H), 4.11-3.89 (m, 3H), 3.55 (d, J = 14.0 Hz, 1H), 3.18 (d, J = |
| | 13.6 Hz, 1H), 3.10-2.92 (m, 4H), 2.72-2.55 (m, 4H), 2.41-2.31 (m, 4H), 2.09-1.97 (m, |
| | 1H), 1.94-1.72 (m, 2H), 1.71-1.61 (m, 1H) |
| 13B | ¹H NMR (400 MHz, DMSO-d₆, ppm) δ 6.82 (s, 2H), 6.48 (s, 1H), 4.89 (s, 2H), 4.82 (d, J = |
| | 12.8 Hz, 1H), 4.46 (d, J = 3.6 Hz, 2H), 3.97 (s, 2H), 3.83 (dd, J = 10.4, 2.4 Hz, 1H), 3.55 (d, |
| | J = 14.0 Hz, 1H), 3.19 (d, J = 14.0 Hz, 1H), 3.09-2.94 (m, 4H), 2.78 (t, J = 11.2 Hz, 1H), 2.71- |
| | 2.62 (m, 1H), 2.61-2.53 (m, 2H), 2.41-2.30 (m, 4H), 2.09-1.94 (m, 1H), 1.93-1.73 (m, |
| | 2H), 1.72-1.61 (m, 1H) |
| 14 | ¹H NMR (300 MHz, DMSO-d₆, ppm) δ 6.98 (d, J = 2.0 Hz, 1H), 6.70 (s, 2H), 6.42 (s, 1H), |
| | 4.93 (s, 2H), 4.88-4.70 (m, 1H), 4.70-4.50 (m, 1H), 4.50-4.35 (m, 1H), 4.20-3.95 (m, 3H), |
| | 3.83-3.71 (m, 2H), 3.63 (d, J = 14.2 Hz, 1H), 3.21-3.00 (m, 3H), 2.65-2.55 (m, 3H), 2.41- |
| | 2.32 (m, 3H), 2.01-1.60 (m, 8H). |
| 15 | ¹H NMR (300 MHz, DMSO-d₆, ppm) δ 7.76 (dd, J = 8.9, 2.8 Hz, 1H), 7.04 (d, J = 2.8 Hz, 1H), |
| | 6.87 (s, 2H), 6.58 (d, J = 8.8 Hz, 1H), 4.93 (s, 2H), 4.79 (dd, J = 29.2, 12.8 Hz, 1H), 4.61 (t, |
| | J = 12.0 Hz, 1H), 4.50-4.30 (m, 1H), 4.16-3.90 (m, 3H), 3.80-3.50 (m, 3H), 3.20-3.00 (m, |
| | 3H), 2.70-2.55 (m, 2H), 2.40-2.30 (m, 1H), 2.05-1.55 (m, 9H). |
| 16A | ¹H NMR (300 MHz, DMSO-d₆, ppm) δ 8.15 (s, 1H), 7.90 (dd, J = 8.8, 5.1 Hz, 1H), 7.31 (s, |
| | 1H), 7.16 (t, J = 9.2 Hz, 1H), 4.93-4.79 (m, 3H), 4.70-4.60 (m, 1H), 4.47 (dd, J = 13.1, 7.7 |
| | Hz, 1H), 4.10-3.91 (m, 3H), 3.70-3.50 (m, 6H), 3.25-2.96 (m, 4H), 2.62-2.55 (m, 1H), |
| | 2.35-2.30 (m, 1H), 2.03-1.50 (m, 8H). |
| 16B | ¹H NMR (300 MHz, DMSO-d₆, ppm) δ 8.14 (s, 1H), 7.90 (dd, J = 8.8, 5.1 Hz, 1H), 7.32 (s, |
| | 1H), 7.17 (t, J = 9.2 Hz, 1H), 4.86 (d, J = 26.4 Hz, 3H), 4.70-4.59 (m, 1H), 4.47 (dd, J = |
| | 13.1, 7.9 Hz, 1H), 4.12-3.90 (m, 3H), 3.64-3.45 (m, 6H), 3.25-2.93 (m, 4H), 2.63-2.55 (m, |
| | 1H), 2.40-2.30 (m, 1H), 1.99-1.56 (m, 8H). |
| | ¹H NMR (400 MHz, DMSO-d₆, ppm) δ 8.18 (s, 1H), 7.98 (dd, J = 8.8, 5.2 Hz, 1H), 7.23 (dd, |
| | J = 9.8, 8.8 Hz, 1H), 4.95-4.82 (m, 3H), 4.70-4.60 (m, 1H), 4.45 (dd, J = 13.0, 8.3 Hz, 1H), 4.09 |
| | (d, J = 8.1 Hz, 1H), 3.98 (q, J = 10.4 Hz, 2H), 3.64-3.43 (m, 6H), 3.19 (d, J = 14.1 Hz, 1H), |
| | 3.07 (d, J = 12.8 Hz, 1H), 3.03-2.92 (m, 1H), 2.83 (br, 1H), 2.62-2.53 (m, 2H), 2.35 (d, J = |
| | 15.5 Hz, 1H), 1.97 (dd, J = 11.5, 6.3 Hz, 1H), 1.91-1.61 (m, 6H), 1.60-1.45 (m, 1H) |
| 17A | ¹H NMR (400 MHz, DMSO-d₆, ppm) δ 8.19 (s, 1H), 7.98 (dd, J = 8.8, 5.2 Hz, 1H), 7.22 (dd, |
| 17B | J = 9.7, 8.8 Hz, 1H), 5.10-4.80 (m, 3H), 4.69-4.59 (m, 1H), 4.44 (dd, J = 13.0, 8.1 Hz, 1H), |
| | 4.08-3.91 (m, 3H), 3.62-3.43 (m, 6H), 3.19 (d, J = 14.0 Hz, 1H), 3.08 (d, J = 12.9 Hz, 1H), |
| | 3.01-2.92(m, 1H), 2.82 (br, 1H), 2.65-2.53 (m, 2H), 2.35 (d, J = 15.5 Hz, 1H), 2.03-1.92 |
| | (m, 1H), 1.92-1.62 (m, 6H), 1.60-1.50 (m, 1H) |
| 18A | ¹H NMR (300 MHz, DMSO-d₆, ppm) δ 8.20 (s, 1H), 8.01 (dd, J = 8.8, 5.2 Hz, 1H), 7.25 (t, J = |
| | 9.3 Hz, 1H), 4.86 (dd, J = 13.0, 2.4 Hz, 1H), 4.62 (dd, J = 13.1, 2.6 Hz, 1H), 4.43 (dd, J = |
| | 13.0, 8.0 Hz, 1H), 4.20-4.01 (m, 3H), 3.90-3.70 (m, 2 H), 3.63 (s, 3 H), 3.30-2.97 (m, 4H), |
| | 2.78-2.60 (m, 1H), 2.43-2.26 (m, 2H), 2.09-1.60 (m, 8H) |
| 19 | ¹H NMR (300 MHz, DMSO-d₆, ppm) δ 6.79 (s, 2H), 6.61 (dd, J = 6.1, 2.4 Hz, 1H), 6.45 (s, |
| | 1H), 4.95-4.73 (m, 3H), 4.50-4.35 (m, 1H), 4.28-4.20 (m, 1H), 4.04-3.88 (m, 3H), 3.56 (d, |
| | J = 13.7 Hz, 2H), 3.44 (s, 1H), 3.19 (d, J = 14.0 Hz, 1H), 3.10-2.95 (m, 2H), 2.65-2.55 (m, |
| | 2H), 2.42-2.29 (m, 4H), 2.03-1.47 (m, 8H) |
| 20 | ¹H NMR (300 MHz, DMSO-d₆, ppm) δ 6.50 (d, J = 0.9 Hz, 1H), 6.21 (s, 2H), 4.93-4.80 (m, |
| | 3H), 4.59 (dd, J = 13.1, 2.7 Hz, 1H), 4.31 (dd, J = 13.0, 8.2 Hz, 1H), 4.10-4.02 (m, 1H), 3.97 |
| | (q, J = 10.5 Hz, 2H), 3.55 (d, J = 13.5 Hz, 2H), 3.45 (d, J = 5.8 Hz, 1H), 3.19 (d, J = 14.0 Hz, |
| | 1H), 3.11-2.95 (m, 2H), 2.65-2.54 (m, 2H), 2.35 (d, J = 15.7 Hz, 1H), 2.30-2.22 (m, 3H), |
| | 2.02-1.92 (m, 1H), 1.92-1.75 (m, 2H), 1.74-1.61 (m, 4H), 1.60-1.50 (m, 1H) |
| 21 | ¹H NMR (300 MHz, DMSO-d₆, ppm) δ 6.84 (s, 2H), 6.47 (s, 1H), 5.28 (d, J = 54.2 Hz, 1H), |
| | 4.76 (dd, J = 12.9, 2.4 Hz, 1H), 4.55 (dd, J = 13.2, 2.9 Hz, 1H), 4.36 (dd, J = 13.1, 7.2 Hz, |
| | 1H), 4.09 (d, J = 10.3 Hz, 1H), 4.02-3.88 (m, 2H), 3.57 (s, 1H), 3.46 (d, J = 5.8 Hz, 1H), |
| | 3.20-2.97 (m, 4H), 2.91-2.78 (m, 1H), 2.35 (s, 3 H), 2.18-2.14 (m, 1H), 2.08-1.91 (m, |
| | 2H), 1.91-1.47 (m, 7H). |
| 22A | ¹H NMR (400 MHz, DMSO-d₆, ppm) δ 7.80 (d, J = 8.8 Hz, 1H), 6.96 (s, 2H), 6.61 (d, J = 8.8 |
| 22B | Hz, 1H), 4.86 (dd, J = 13.2, 2.4 Hz, 1H), 4.59 (dd, J = 13.2, 2.8 Hz, 1H), 4.30 (dd, J = 12.8, |
| | 8.4 Hz, 1H), 4.15-4.01 (m, 3H), 3.61-3.52 (m, 1H), 3.49-3.41 (m, 1H), 3.39-3.35 (m, 1H), |
| | 3.17-3.01 (m, 3H), 2.81-2.67 (m, 2H), 2.49-2.33 (m, 2H), 2.09-1.99 (m, 1H), 1.98-1.86 |
| | (m, 1H), 1.83-1.73 (m, 2H), 1.72-1.61 (m, 3H), 1.60-1.49 (m, 1H). |
| | ¹H NMR (400 MHz, DMSO-d₆, ppm) δ 7.80 (d, J = 8.8 Hz, 1H), 6.96 (s, 2H), 6.61 (d, J = 8.8 |
| | Hz, 1H), 4.77 (dd, J = 12.8, 2.0 Hz, 1H), 4.54 (d, J = 2.4 Hz, 1H), 4.47-4.29 (m, 1H), 4.12 (d, |
| | J = 10.8 Hz, 1H), 4.05-3.97 (m, 2H), 3.63-3.53 (m, 1H), 3.52-3.44 (m, 1H), 3.31-3.27 (m, 1H), |
| | 3.17-3.01 (m, 3H), 2.91 (s, 1H), 2.78-2.67 (m, 1H), 2.47-2.31 (m, 2H), 2.09-1.97 (m, 1H), |
| | 1.96-1.83 (m, 1H), 1.82-1.72 ( m, 3H), 1.71-1.49 (m, 3H). |
| 23 | ¹H NMR (400 MHz, DMSO-d₆) δ 6.84 (s, 2H), 6.46 (s, 1H), 5.27 (d, J = 54 Hz, 1H), 5.15- |
| | 5.12 (m, 1H), 4.34-4.30 (m, 1H), 4.07 (d, J = 10 Hz, 1H), 3.96 (t, J = 9 Hz, 2H), 3.55 (s, |
| | 1H), 3.38 (d, J = 6 Hz, 1H), 3.33-3.06 (m, 2H), 3.02-3.00 (m, 2H), 2.849-2.79 (m, 1H), |
| | 2.36 (s, 3H), 2.13-2.11 (m, 1H), 2.04-1.98 (m, 2H), 1.87-1.72 (m, 4H), 1.62-1.58 (m, |
| | 2H), 1.53-1.47 (m, 4H). |
| 24 | ¹H NMR (400 MHz, DMSO-d₆) δ 6.83 (s, 2H), 6.47 (s, 1H), 5.27 (d, J = 54.4 Hz, 1H), 5.16- |
| | 5.13 (m, 1H), 4.32-4.28 (m, 1H), 4.05-3.94 (m, 3H), 3.55 (d, J = 4.4 Hz, 1H), 3.39-3.33 |
| | (m, 1H), 3.09-3.07 (m, 2H), 3.02-3.00 (m, 2H), 2.85-2.79 (m, 2H), 2.35 (s, 3H), 2.14- |
| | 2.12 (m, 1H), 2.08-2.00 (m, 2H), 1.92-1.73 (m, 4H), 1.60-1.46 (m, 6H). |
| 25 | ¹H NMR (400 MHz, DMSO-d6) δ 6.84 (s, 2H), 6.47 (s, 1H), 5.31-5.06 (m, 2H), 4.43 (dd, J = |
| | 11.1, 4.6 Hz, 1H), 4.33 (dq, J = 9.2, 6.2 Hz, 1H), 4.19 (dd, J = 11.0, 5.7 Hz, 1H), 3.98 (d, J = |
| | 9.5 Hz, 1H), 3.53 (d, J = 5.3 Hz, 1H), 3.51-3.41 (m, 1H), 3.39 (dd, J = 5.7, 2.6 Hz, 1H), 3.02 |
| | (d, J = 12.6 Hz, 1H), 2.97-2.70 (m, 2H), 2.40 (s, 3H), 2.36 (d, J = 2.1 Hz, 3H), 2.21-2.04 |
| | (m, 1H), 2.02-1.79 (m, 2H), 1.61 (t, J = 9.4 Hz, 2H), 1.49 (d, J = 6.3 Hz, 4H). |
| 26 | ¹H NMR (300 MHz, DMSO-d6) δ 6.85 (s, 2H), 6.47 (s, 1H), 5.15 (dd, J = 12.7, 2.7 Hz, 1H), |
| | 4.70-4.55 (m, 2H), 4.50 (s, 1H), 4.41-4.34 (m, 1H), 3.99 (d, J = 9.4 Hz, 1H), 3.60 (s, 2H), |
| | 3.56 (s, 1H), 3.41 (d, J = 5.8 Hz, 1H), 3.02 (d, J = 12.6 Hz, 1H), 2.37 (s, 3H), 1.91-1.79 (m, |
| | 3H), 1.64-1.43 (m, 8H). |
| 27 | ¹H NMR (300 MHz, DMSO-d6) δ 6.85 (s, 2H), 6.47 (s, 1H), 5.16 (d, J = 12.7, 2.7 Hz, 1H), |
| | 4.39-4.33 (m, 1H), 4.28-4.13 (m, 2H), 3.98 (d, J = 9.4 Hz, 1H), 3.55 (d, J = 5.1 Hz, 1H), |
| | 3.40-3.36 (m, 1H), 3.17 (s, 3H), 3.02 (d, J = 12.4 Hz, 1H), 2.79 (s, 1H), 2.37 (s, 3H), 1.97- |
| | 1.89 (m, 1H), 1.71-1.52 (m, 6H), 1.28-1.19 (m, 6H). |
| 28 | ¹H NMR (400 MHz, DMSO-d₆) δ 6.83 (s, 2H), 6.47 (s, 1H), 5.16 (dd, J = 12.7, 2.7 Hz, 1H), |
| | 4.41-4.20 (m, 3H), 3.98 (d, J = 9.6 Hz, 1H). 3.58-3.52 (m, 1H), 3.46-3.37 (m, 3H), 3.08- |
| | 2.91 (m, 2H), 2.89-2.75 (m, 2H), 2.42-2.29 (m, 4H), 2.20-1.96 (m, 3H), 1.94-1.84 (m, |
| | 1H), 1.69-1.45 (m, 8H). |
| 29 | ¹H NMR (400 MHz, DMSO-d₆) δ 6.83 (s, 2H), 6.47 (s, 1H), 5.16 (dd, J = 12.7, 2.7 Hz, 1H), |
| | 4.40-4.25 (m, 2H), 4.15 (d, J = 10.8 Hz, 1H), 3.98 (d, J = 9.6 Hz, 1H), 3.58-3.51 (m, 1H), |
| | 3.50-3.43 (m, 1H), 3.39 (d, J = 6.0 Hz, 1H), 3.23 (dd, J = 17.6, 8.8 Hz, 1H), 3.08-2.98 (m, |
| | 2H), 2.70-2.61 (m, 1H), 2.41-2.32 (m, 4H), 2.29-2.20 (m, 2H), 2.13-2.06 (m, 1H), 1.95- |
| | 1.74 (m, 3H), 1.65-1.46 (m, 6H). |
| 30A | ¹H NMR (400 MHz, DMSO-d₆) δ 6.84 (s, 2H), 6.46 (s, 1H), 5.15 (dd, J = 12.8, 2.4 Hz, 1H), |
| 30B | 4.41-4.22 (m, 3H), 3.98 (d, J = 9.6 Hz, 1H), 3.61-3.51 (m, 2H), 3.40 (d, J = 6.0 Hz, 1H), |
| | 3.27-2.98 (m, 4H), 2.71-2.59 (m, 2H), 2.42-2.27 (m, 6H), 1.95-1.83 (m, 1H), 1.49- |
| | 1.69 (m, 5H). |
| | ¹H NMR (400 MHz, DMSO-d₆) δ 6.84 (s, 2H), 6.46 (s, 1H), 5.15 (dd, J = 12.8, 2.4 Hz, 1H), |
| | 4.41-4.22 (m, 3H), 3.98 (d, J = 9.6 Hz, 1H), 3.61-3.51 (m, 2H), 3.40 (d, J = 6.0 Hz, 1H), |
| | 3.27-2.98 (m, 4H), 2.71-2.59 (m, 2H), 2.42-2.27 (m, 6H), 1.95-1.83 (m, 1H), 1.49- |
| | 1.69 (m, 5H). |
| 31 | ¹H NMR (300 MHz, DMSO-d6, ppm) δ 6.86 (s, 2H), 6.48 (s, 1H), 5.16 (dd, J = 12.8, 2.7 Hz, |
| | 1H), 4.85-4.64 (m, 6H), 4.35 (dq, J = 9.6, 6.2 Hz, 1H), 3.99 (d, J = 9.4 Hz, 1H), 3.55 (s, 1H), |
| | 3.40 (d, J = 6.0 Hz, 1H), 3.02 (d, J = 12.6 Hz, 1H), 2.37 (d, J = 2.3 Hz, 3H), 1.94-1.81 (m, |
| | 1H), 1.69-1.58 (m, 2H), 1.50 (d, J = 6.2 Hz, 4H). |
| 32 | ¹H NMR (300 MHz, DMSO-d6, ppm) δ 6.85 (s, 2H), 6.47 (s, 1H), 5.96 (tdd, J = 56.0, 5.2, 3.3 |
| | Hz, 1H), 5.15 (dd, J = 12.7, 2.7 Hz, 1H), 4.33 (dd, J = 10.6, 5.8 Hz, 3H), 3.98 (d, J = 9.5 Hz, |
| | 1H), 3.69-3.54 (m, 2H), 3.43-3.34 (m, 1H), 3.15-2.95 (m, 3H), 2.90-2.70 (m, 2H), 2.37 (d, |
| | J = 2.3 Hz, 3H), 2.12-1.96 (m, 2H), 1.89 (s, 1H), 1.70-1.40 (m, 6H). |
| 33 | ¹H NMR (400 MHz, DMSO-d6, ppm) δ 6.85 (s, 2H), 6.48 (s, 1H), 5.22-5.14 (m, 1H), 4.47- |
| | 4.28 (m, 5H), 3.99 (d, J = 9.5 Hz, 1H), 3.55 (d, J = 5.3 Hz, 1H), 3.40 (d, J = 6.1 Hz, 1H), 3.03 |
| | (d, J = 12.6 Hz, 1H), 2.90 (br, 1 H), 2.68-2.59 (m, 1H), 2.46-2.35 (m, 4H), 1.92 (d, J = 8.8 |
| | Hz, 1H), 1.61 (d, J = 9.9 Hz, 2H), 1.50 (d, J = 6.3 Hz, 4H), 1.44 (s, 3H). |
| 34 | ¹H NMR (300 MHz, DMSO-d6, ppm) δ 6.83 (s, 2H), 6.49-6.42 (m, 1H), 5.14 (dd, J = 12.8, |
| | 2.7 Hz, 1H), 4.49 (dd, J = 5.9, 4.0 Hz, 2H), 4.43-4.24 (m, 5H), 3.96 (d, J = 9.4 Hz, 1H), 3.53 |
| | (d, J = 4.6 Hz, 1H), 3.38 (d, J = 5.8 Hz, 1H), 3.00 (d, J = 12.6 Hz, 1H), 2.90 (br, 1H), 2.38- |
| | 2.31 (m, 3H), 1.88 (d, J = 8.6 Hz, 1H), 1.61 (t, J = 9.3 Hz, 2H), 1.47 (d, J = 6.2 Hz, 4H), 1.34 |
| | (s, 3H). |
| 35 | ¹H NMR (300 MHz, DMSO-d6, ppm) δ 6.82 (s, 2H), 6.45 (s, 1H), 5.19-5.08 (m, 1H), 4.50- |
| | 4.20 (m, 2H), 4.05 (dd, J = 10.8, 6.0 Hz, 1H), 3.96 (d, J = 9.5 Hz, 1H), 3.60-3.40 (m, 6H), |
| | 3.12 (t, J = 10.4 Hz, 1H), 3.05-2.75 (m, 5H), 2.35 (d, J = 2.3 Hz, 3H), 2.11-1.96 (m, 1H), |
| | 1.90-1.80 (m, 1H), 1.74 (t, J = 4.9 Hz, 1H), 1.62-1.50 (m, 3H), 1.47 (d, J = 6.2 Hz, 3H), 1.32- |
| | 1.25 (m, 1H). |
| 36 | ¹H NMR (300 MHz, DMSO-d₆, ppm) δ 6.95-6.56 (m, 3H), 6.45 (s, 1H), 5.12 (d, J = 12.6, 2.6 |
| | Hz, 1H), 4.41-4.22 (m, 1H), 4.05 (d, J = 10.5 Hz, 1H), 3.95 (m, 2H), 3.77-3.63 (m, 1H), |
| | 3.53 (s, 1H), 3.38 (d, J = 5.9 Hz, 1H), 3.24 (s, 2H), 2.98 (m, 2H), 2.56 (d, J = 8.2 Hz, 2H), |
| | 2.41-2.23 (m, 4H), 2.00-1.52 (m, 8H), 1.47 (d, J = 6.2 Hz, 3H). |
| 37 | ¹H NMR (300 MHz, DMSO-d₆) 0 6.84 (s, 2H), 6.48 (s, 1H), 5.59 (m, 1H), 5.17 (m, 1H), 4.41- |
| | 4.26 (m, 2H), 4.12 (d, J = 10.9 Hz, 1H), 3.98 (d, J = 9.4 Hz, 1H), 3.53 (m, 2H), 3.40 (d, J = |
| | 5.7 Hz, 1H), 3.13-2.94 (m, 3H), 2.89 (t, J = 8.4 Hz, 1H), 2.78 (s, 1H), 2.47-2.22 (m, 6H), |
| | 2.16-2.11 (m, 1H), 1.88 (t, J = 9.6 Hz, 1H), 1.60 (m, 3H), 1.49 (d, J = 6.2 Hz, 3H). |
| 38A | ¹H NMR (400 MHz, DMSO-d₆) δ 7.80 (d, J = 9.0 Hz, 1H), 6.97 (s, 2H), 6.61 (d, J = 8.8 Hz, |
| 38B | 1H), 5.18-5.14 (m, 1H), 4.41-4.30 (m, 2H), 4.27 (d, J = 11.0 Hz, 1H), 3.99 (d, J = 9.4 Hz, |
| | 1H), 3.56-3.52 (m, 2H), 3.40 (d, J = 6.1 Hz, 1H), 3.22-3.08 (m, 1H), 3.13 (d, J = 4.7 Hz, |
| | 1H), 3.10-2.98 (m, 2H), 2.87 (s, 1H), 2.70-2.56 (m, 1H), 2.50-2.25 (m, 3H), 1.93-1.84 |
| | (m, 1H), 1.65-1.60 (m, 2H), 1.48 (d, J = 6.3 Hz, 4H). |
| | ¹H NMR (400 MHz, DMSO-d₆) δ 7.80 (d, J = 8.9 Hz, 1H), 6.97 (s, 2H), 6.61 (d, J = 8.8 Hz, |
| | 1H), 5.18-5.14 (m, 1H), 4.41-4.23 (m, 3H), 3.99 (d, J = 9.4 Hz, 1H), 3.55-3.51 (m, 2H), |
| | 3.40 (d, J = 6.2 Hz, 1H), 3.23-3.20 (m, 1H), 3.13 (d, J = 4.3 Hz, 1H), 3.09-2.98 (m, 2H), |
| | 2.68-2.58 (m, 1H), 2.49-2.26 (m, 3H), 1.95-1.84 (m, 1H), 1.66-1.51 (m, 2H), 1.48 (d, |
| | J = 6.3 Hz, 4H). |
| 39 | ¹H NMR (400 MHz, DMSO-d₆) δ 7.80 (d, J = 9.0 Hz, 1H), 6.97 (s, 2H), 6.61 (d, J = 8.8 Hz, |
| | 1H), 5.43-5.02 (m, 2H), 4.35-4.32 (m, 1H), 4.09 (d, J = 10.3 Hz, 1H), 4.00-3.94 (m, 2H), |
| | 3.57 (s, 1H), 3.40 (d, J = 6.3 Hz, 2H), 3.17-3.05 (m, 2H), 3.02 (d, J = 12.5 Hz, 2H), 2.83- |
| | 2.67 (m, 1H), 2.19-2.09 (m, 1H), 2.05 (s, 1H), 2.00 (d, J = 10.6 Hz, 1H), 1.96-1.82 (m, |
| | 2H), 1.82-1.70 (m, 2H), 1.70-1.53 (m, 2H), 1.56-1.41 (m, 4H). |
| 40 | ¹H NMR (400 MHz, DMSO-d₆, ppm) 7.80 (d, J = 8.9 Hz, 1H), 6.92 (d, J = 43.2 Hz, 2H), |
| | 6.70-6.55 (m, 2H), 5.14 (dd, J = 12.7, 2.8 Hz, 1H), 4.43-4.26 (m, 1H), 4.10-3.91 (m, 2H), |
| | 3.70 (d, J = 14.7 Hz, 2H), 3.54 (d, J = 5.7 Hz, 1H), 3.39 (d, J = 6.4 Hz, 1H), 3.28 (d, J = 15.2 |
| | Hz, 1H), 3.10-2.94 (m, 2H), 2.61-2.53 (m, 2H), 2.32 (d, J = 14.7 Hz, 1H), 2.01-1.53 (m, |
| | 8H), 1.48 (d, J = 6.3 Hz, 3H). |
| 41A | ¹H NMR (400 MHz, DMSO-d₆) δ 7.80 (d, J = 8.8 Hz, 1H), 6.99 (s, 2H), 6.60 (d, J = 8.8 Hz, 1H), |
| | 5.16-5.13 (m, 1H), 4.36-4.32 (m, 1H), 4.14-4.10 (m, 2H), 3.98 (d, J = 9.2 Hz, 1H), 3.55 (s, |
| | 1H), 3.39-3.33 (m, 2H), 3.10-2.99 (m, 3H), 2.90-2.65 (m, 2H), 2.60-2.50 (m, 1H), 2.10-1.40 |
| | (m, 14H). |
| 41B | ¹H NMR (400 MHz, DMSO-d₆) δ 7.80 (d, J = 9.2 Hz, 1H), 6.99 (s, 2H), 6.60 (d, J = 9.2 Hz, 1H), |
| | 5.16-5.13 (m, 1H), 4.40-4.30 (m, 1H), 4.18-4.08 (m, 2H), 4.00 (d, J = 9.2 Hz, 1H), 3.55 (s, |
| | 1H), 3.40-3.33 (m, 2H), 3.10-2.99 (m, 3H), 2.90-2.65 (m, 2H), 2.60-2.50 (m, 1H), 2.10-1.40 |
| | (m, 14H). |
| 42A | ¹H NMR (400 MHz, DMSO-d₆, ppm) δ 8.52 (s, 1H), 6.84 (s, 2H), 6.46 (s, 1H), 5.25 (m, 1H), |
| | 4.36 (m, 1H), 3.99 (d, J = 9.6 Hz, 1H), 3.54-3.48 (m, 1H), 3.41-3.35 (m, 1H), 2.99 (d, J = |
| | 12.5 Hz, 1H), 2.35 (d, J = 2.3 Hz, 3H), 1.90-1.80 (m, 1H), 1.66-1.51 (m, 2H), 1.51-1.38 |
| | (m, 4H) |
| 42B | ¹H NMR (400 MHz, DMSO-d₆, ppm) δ 8.53 (s, 1H), 6.83 (s, 2H), 6.47 (s, 1H), 5.27 (m, 1H), |
| | 4.37 (m, 1H), 4.05 (d, J = 9.7 Hz, 1H), 3.61 (s, 1H), 3.50-3.44 (m, 1H), 3.04 (d, J = 12.8 Hz, |
| | 1H), 2.51-2.37 (m, 3H), 1.93 (s, 1H), 1.73-1.53 (m, 3H), 1.52-1.40 (m, 3H) |
| 43A | ¹H NMR (400 MHz, DMSO-d₆, ppm): δ 8.19 (s, 1H), 7.97 (dd, J = 8.9, 5.2 Hz, 1H), 7.21 (t, |
| 43B | J = 9.3 Hz, 1H), 5.35-5.19 (m, 2H), 4.46 (dd, J = 9.4, 6.2 Hz, 1H), 4.09-3.99 (m, 3H), 3.60 (s, |
| | 4H), 3.58 (d, J = 5.3 Hz, 1H),3.12-2.98 (m, 4H), 2.83 (t, J = 8.1 Hz, 1H), 2.20-2.10 (m, |
| | 3H), 1.93-1.84 (m, 4H), 1.52 (d, J = 6.2 Hz, 6H) |
| | ¹H NMR (400 MHz, DMSO-d₆, ppm): δ 8.17 (s, 1H), 7.97 (dd, J = 8.8, 5.2 Hz, 1H), 7.23 (dd, |
| | J = 9.7, 8.8 Hz, 1H), 5.35 (d, J = 3.9 Hz, 1H), 5.15 (dd, J = 12.7, 2.7 Hz, 1H), 4.51 (dd, J = |
| | 9.8, 6.2 Hz, 1H), 4.11-3.96 (m, 3H), 3.57 (d, J = 5.7 Hz, 1H), 3.46 (s, 4H), 3.12-2.99 (m, |
| | 4H), 2.82 (dd, J = 8.3, 7.8 Hz, 1H), 2.21-2.11 (m, 1H), 2.05 (d, J = 3.1 Hz, 1H), 2.00 (s, 1H), |
| | 1.93-1.82 (dd, J = 11.8, 8.0 Hz, 2H), 1.79 (dd, J = 13.2, 6.0 Hz, 2H), 1.65 (dd, J = 11.8, 8.0 |
| | Hz, 2H), 1.65-1.47 (m, 4H) |
| 44 | ¹H NMR (300 MHz, DMSO-d₆) δ 9.62 (d, J = 14.5 Hz, 1H), 7.28 (t, J = 8.6 Hz, 1H), 7.10 (dd, |
| | J = 20.0, 7.6 Hz, 1H), 7.02-6.84 (m, 2H), 5.38-5.19 (d, J = 57 Hz, 1H), 5.18-5.10 (m, 1H), |
| | 4.30-4.25 (m, 1H), 4.05 (dd, J = 30, 10.5 Hz, 2H), 4.04-3.94 (m, 1H), 3.57 (d, J = 5.4 Hz, |
| | 1H), 3.41 (d, J = 4.8 Hz, 1H), 3.19-2.97 (m, 4H), 2.90-2.75 (m, 1H), 2.25-1.50(m, 13H) |
| 45A | ¹H NMR (300 MHz, DMSO-d6, ppm) δ 6.81 (s, 2H), 6.50-6.43 (m, 1H), 5.40-5.15 (m, 1H), |
| 45B | 4.89 (dd, J = 13.6, 5.4 Hz, 1H), 4.60 (dt, J = 12.7, 5.3 Hz, 2H), 4.51-4.41 (m, 2H), 4.22 (s, |
| | 1H), 4.07 (d, J = 10.3 Hz, 1H), 3.95 (d, J = 10.3 Hz, 1H), 3.26 (s, 1H), 3.18-2.96 (m, 5H), |
| | 2.90-2.75 (m, 1H), 2.69 (t, J = 6.9 Hz, 2H), 2.60-2.50 (m, 1H), 2.39-2.31 (m, 4H), 2.22- |
| | 2.09 (m, 1H), 2.09-1.96 (m, 2H), 1.90-1.70 (m, 6H), 1.57-1.43 (m, 2H), 1.41-1.31 (m, 1H) |
| | ¹H NMR (400 MHz, DMSO-d6, ppm) δ 6.82 (s, 2H), 6.47 (s, 1H), 5.40-5.20 (m, 1H), 4.96 (dd, |
| | J = 13.7, 5.3 Hz, 1H), 4.66-4.57 (m, 2H), 4.49 (t, J = 6.0 Hz, 1H), 4.42 (dd, J = 13.1, 1.7 Hz, |
| | 1H), 4.22 (d, J = 4.0 Hz, 1H), 4.13 (d, J = 10.4 Hz, 1H), 3.95 (d, J = 10.4 Hz, 1H), 3.35-3.25 |
| | (m, 1 H), 3.15-3.00 (m, 5H), 2.84 (q, J = 8.3 Hz, 1H), 2.71 (t, J = 6.9 Hz, 2H), 2.60-2.50 (m, |
| | 1H), 2.40-2.33 (m, 4H), 2.21-2.10 (m, 1H), 2.10-1.90 (m, 2H), 1.90-1.70 (m, 6H), 1.61- |
| | 1.42 (m, 2H), 1.42-1.35 (m, 1H) |
| 46 | ¹H NMR (300 MHz, DMSO-d6, ppm) δ 6.83 (s, 2H), 6.48 (s, 1H), 5.42-5.15 (m, 1H), 4.93 (dd, |
| | J = 13.6, 5.5 Hz, 1H), 4.61 (dd, J = 13.0, 4.4 Hz, 1H), 4.42 (d, J = 12.6 Hz, 1H), 4.22 (s, 1H), |
| | 4.13 (d, J = 10.4 Hz, 1H), 3.93 (d, J = 10.3 Hz, 1H), 3.15-3.06 (m, 2H), 3.06-2.91 (m, 3H), |
| | 2.83 (d, J = 6.7 Hz, 1H), 2.44-2.32 (m, 8H), 2.23-2.10 (m, 1H), 2.08-1.92 (m, 2H), 1.88- |
| | 1.72 (m, 4H), 1.65-1.35 (m, 4H) |
| 47 | ¹H NMR (300 MHz, DMSO-d6, ppm) δ 6.84 (s, 2H), 6.48 (s, 1H), 5.40-5.15 (m, 1H), 4.93 (dd, |
| | J = 13.6, 5.3 Hz, 1H), 4.72-4.55 (m, 3H), 4.41 (d, J = 12.7 Hz, 1H), 4.30 (t, J = 6.0 Hz, 2H), |
| | 4.18 (s, 1H), 4.12 (d, J = 10.3 Hz, 1H), 3.93 (d, J = 10.3 Hz, 1H), 3.28-3.06 (m, 4H), 3.00 |
| | (s, 3H), 2.93 (d, J = 7.6 Hz, 2H), 2.90-2.78 (m, 1H), 2.56 (s, 1H), 2.39-2.32 (m, 4H), 2.15 |
| | (d, J = 4.6 Hz, 1H), 2.09-1.90 (m, 2H), 1.88-1.72 (m, 4H), 1.60-1.34 (m, 3H) |
| 48 | ¹H NMR (300 MHz, DMSO-d6, ppm) δ 6.81 (s, 2H), 6.46 (s, 1H), 5.40-5.13 (m, 1H), 4.92 (dd, |
| | J = 13.6, 5.2 Hz, 1H), 4.61 (dd, J = 13.0, 4.1 Hz, 1H), 4.40 (d, J = 12.7 Hz, 1H), 4.23 (s, 1H), |
| | 4.10 (d, J = 10.3 Hz, 1H), 3.91 (d, J = 10.3 Hz, 1H), 3.33 (s, 1H), 3.13-2.95 (m, 5H), 2.81 |
| | (q, J = 8.8, 8.2 Hz, 1H), 2.67 (t, J = 6.4 Hz, 2H), 2.55 (t, J = 7.0 Hz, 3H), 2.38-2.30 (m, 4H), |
| | 2.17-2.03 (m, 1H), 1.99 (d, J = 13.4 Hz, 2H), 1.74 (dt, J = 13.9, 7.7 Hz, 6H), 1.60-1.30 (m, |
| | 3H) |
| 49 | ¹H NMR (300 MHz, DMSO-d6, ppm) δ 6.87 (d, J = 17.0 Hz, 2H), 6.65-6.60 (m, 1H), 6.47 (s, |
| | 1H), 4.48 (t, J = 5.9 Hz, 1H), 4.41 (d, J = 12.7 Hz, 1H), 4.20 (s, 1H), 4.06 (d, J = 10.5 Hz, |
| | 1H), 3.99 (d, J = 10.5 Hz, 1H), 3.78 (d, J = 14.6 Hz, 1H), 3.33-3.21 (m, 2H), 3.11-2.80 (m, |
| | 3H), 2.75 (m, 2H), 2.54 (d, J = 15.0 Hz, 3H), 2.41-2.25 (m, 5H), 2.01-1.98 (m, 2H), 1.82- |
| | 1.42 (m, 5H) |
| 50 | ¹H NMR (400 MHz, DMSO-d6, ppm) δ 6.84 (d, J = 17.0 Hz, 2H), 6.68-6.62 (m, 1H), 6.47 (s, |
| | 1H), 4.94 (m, 1H), 4.61 (m, 2H), 4.48 (t, J = 5.9 Hz, 1H), 4.42 (d, J = 12.7 Hz, 1H), 4.22 (s, |
| | 1H), 4.08 (d, J = 10.5 Hz, 1H), 3.95 (d, J = 10.5 Hz, 1H), 3.70 (d, J = 14.6 Hz, 1H), 3.31- |
| | 3.23 (m, 2H), 3.12-2.94 (m, 3H), 2.70 (m, 2H), 2.56 (d, J = 15.0 Hz, 3H), 2.42-2.28 (m, |
| | 5H), 1.94 (m, 1H), 1.82 (m, 5H), 1.73-1.62 (m, 1H), 1.61-1.48 (m, 2H), 1.42 (d, J = 13.6 |
| | Hz, 1H) |
| 51A | ¹H NMR (300 MHz, DMSO-d₆) δ 6.84 (d, J = 13.1 Hz, 2H), 6.48 (s, 1H), 5.40-5.16 (m, 2H), |
| | 4.76-4.58 (m, 1H), 4.18-3.91 (m, 3H), 3.30-3.19 (m, 2H), 3.14-3.05 (m, 2H), 3.03- |
| | 2.91 (m, 3H), 2.86-2.78 (m, 1H), 2.37-2.35 (m, 3H), 2.25-2.06 (m, 2H), 2.06-1.97 (m, |
| | 2H), 1.88-1.67 (m, 5H), 1.60-1.51 (m, 2H), 1.34-1.27 (m, 4H) |
| 52A | ¹H NMR (300 MHz, DMSO-d₆) δ 6.85 (s, 2H), 6.46 (s, 1H), 5.27 (d, J = 54.4 Hz, 1H), 5.10- |
| 52B | 5.02 (m, 1H), 4.75-4.57 (m, 3H), 4.28-4.23 (m, 2H), 4.10 (d, J = 10.4 Hz, 1H), 4.05-3.84 |
| | (m, 2H), 3.41 (d, J = 13.6 Hz, 1H), 3.18-2.96 (m, 5H), 2.90 (d, J = 7.4 Hz, 3H), 2.82 (d, J = |
| | 7.4 Hz, 1H), 2.54 (d, J = 10.1 Hz, 1H), 2.34 (d, J = 2.1 Hz, 3H), 2.20 (s, 1H), 2.17-2.10 (m, |
| | 1H), 2.09-1.95 (m, 2H), 1.87-1.71 (m, 4H), 1.62-1.48 (m, 2H), 1.37-1.29 (d, J = 6.9 |
| | Hz, 1H), 1.26 (d, J = 6.5 Hz, 3H) |
| | ¹H NMR (300 MHz, DMSO-d₆) δ 6.81 (s, 2H), 6.46 (s, 1H), 5.40-5.12 (m, 1H), 5.06-4.99 |
| | (m, 1H), 4.69-4.50 (m, 3H), 4.29-4.23 (m, 2H), 4.11-3.92 (m, 3H), 3.37 (d, J = 13.6 Hz, |
| | 1H), 3.14-3.05 (m, 4H), 2.99 (s, 1H), 2.94-2.75 (m, 4H), 2.62-2.57 (m, 1H), 2.34 (d, J = |
| | 2.3 Hz, 3H), 2.23-2.09 (m, 2H), 2.09-1.90 (m, 2H), 1.87-1.58 (m, 5H), 1.56-1.08 (m, 5H) |

### Biological Assays

KRAS Biochemical Assay—BODIPY-GDP Exchange TR-FRET. Biochemical compound potencies were assessed by evaluating inhibition of SOS1-mediated nucleotide exchange in KRAS G12D. The SOS1-promoted exchange of fluorescently-labeled GDP (BOPIDY-GDP) was monitored by time-resolved fluorescence resonance energy transfer (TR-FRET). Compounds solubilized in DMSO were dispensed as concentration series into 384-well white assay plates. A preformed complex of biotin-tagged recombinant human KRAS (1.5 nM mutant G12D or wild type) and 0.15 nM terbium-labeled streptavidin (CisBIO) prepared in 10 μL/well assay buffer (20 mM HEPES, pH 7.5, 50 mM NaCl, 10 mM MgCl₂, 0.01% Tween-20 and 1 mM dithiothreitol) was added and allowed to incubate for 10-minutes. The reaction was initiated with the addition of 5 μL of 3 nM recombinant human SOS1 and 300 nM BODIPY-GDP in assay buffer. After a 60-minute incubation, the fluorescence was measured with excitation at 337 nm and emission at 490 and 520 nm. The TR-FRET ratio was determined as the fluorescence at 520 nm divided by the fluorescence at 490 nm multiplied by 10,000. The results were normalized to percent inhibition based on control samples: DMSO (0% inhibition) and control compound at a concentration that inhibits completely (100% inhibition). The normalized TR-FRET results were plotted against compound concentration, and the data fit to a 4-parameter Hill equation to determine the IC₅₀ values.

KRAS 3D-Cell Proliferation Assays. Cellular potencies of compounds were assessed by evaluating inhibition of proliferation in 3D cultures of homozygous mutant KRAS G12D human pancreatic cell lines (AsPC-1 and SW1990) as compared to a KRAS wild type human lung adenocarcinoma cell line (PC-9). Cells were seeded into 384-well black round-bottom, ultra-low attachment assay plates in 50 μL cell growth medium (RPMI-1640 with 10% fetal bovine serum and 2 mM L-glutamine). After overnight incubation at 37° C. and 5% CO₂, compounds solubilized in DMSO were added as dilution series to the wells in a total volume of 150 nL (0.3% DMSO final). The cells were incubated for 7 days at 37° C. and 5% CO₂. Cell proliferation was quantitated by addition of 40 μL/well of CellTiter-Glo® 3D (Promega). This reagent in combination with mechanical disruption releases the cellular ATP to promote activity in a luciferase-based enzyme/substrate chemiluminescent detection system. After a 25-minute incubation under ambient conditions with shaking and an additional 10 minutes without shaking, the contents of the wells were mixed by pipetting up and down repeatedly to disrupt the spheroids, and then the plates were centrifuged to remove bubbles. The plates were incubated for another 15 minutes under ambient conditions, and the luminescence read on a plate reader (e.g. EnVision [PerkinElmer]). The results were normalized to percent inhibition based on the following control samples: DMSO (0% inhibition) and 1 uM staurosporine (100% inhibition). The normalized luminescence results were plotted against compound concentration, and the data fit to a 4-parameter Hill equation to determine the IC₅₀ values.

KRAS G12D NanoBRET Assay. Cellular target engagement was assessed by monitoring inhibition of the Raf-RBD/KRAS G12D interaction in a NanoBRET™ assay (Promega). The assay uses an HCT115 colon cancer cell line stably co-transfected with Raf-RBD fused to NanoLuc® Iuciferase and doxycycline-inducible KRAS G12D fused with a Halotag®. Cells were seeded into 384-well white tissue culture-treated assay plates in 40 μL culture medium (RPMI-1640 with 10% fetal bovine serum, 2 mM glutamine, 2 ng/mL puromycin, and 4 ng/mL blasticidin) with doxycycline to induce KRAS G12D-nanoluciferase expression over 20-24 h at 37° C., 5% CO₂. The culture medium was then removed and replaced with assay medium (Opti-MEM® with 4% fetal bovine serum) and 0.1 μM HaloTag618 ligand. During the subsequent 4 h incubation at 37° C., 5% CO₂, the HaloTag618 ligand binds to the Halotag-labeled KRAS G12D. Compounds solubilized in DMSO were then added as dilution series to the wells in a total volume of 160 nL (0.4% DMSO final), and the plates were incubated overnight at 37° C., 5% CO₂. In the final step, 10 μL NanoGlo substrate in Opti-MEM was added to each well, and the emission read at 460 nm (luciferase signal) and 610 nm (NanoBRET signal) on an EnVision plate reader (PerkinElmer). The Raf-RBD/KRAS G12D interaction results in bioluminescence resonance energy transfer (BRET) from the product of the luciferase reaction in the vicinity of the Raf-RBD to the HaloTag ligand acceptor on the KRAS G12D and generates the NanoBRET signal. Binding of compounds to KRAS G12D and disruption of its interaction with Raf-RBD results in reduction of this signal. The luciferase signal and NanoBRET signal for compound wells were divided by the average corresponding signal for DMSO control wells. Then the NanoBRET ratio was calculated by dividing the control-adjusted NanoBRET signal by the similarly adjusted luciferase signal. The results were normalized to percent inhibition based on control samples: DMSO (0% inhibition) and control compound at a concentration that inhibits completely (100% inhibition). The normalized NanoBRET ratio results were plotted against compound concentration, and the data fit to a 4-parameter Hill equation to determine the IC₅₀ values.

TABLE 5

| | KRas(G12D) | KRas(WT) | SW1990 | AsPC-1 | PC9 |
|---|---|---|---|---|---|
| | GDP | GDP | Prolif | Prolif | Prolif |
| Cmpd | HTRF | HTRF | IC50 | IC50 | IC50 |
| No. | IC50 | IC50 | (G12D) | (G12D) | (WT) |
| 1A | 0.000298 | 0.00172 | 0.00987 | 0.00412 | 1.93 |
| 1B | 0.0886 | 0.385 | 2.46 | 0.904 | 1.43 |
| 2 | 0.000389 | 0.00255 | 0.00236 | 0.00151 | 1.7 |
| 3 | 0.00292 | 0.00833 | 0.511 | 0.263 | 1.84 |
| 4 | 0.00764 | 0.00334 | 1.4 | 1.52 | 9.81 |
| 5 | 0.000361 | 0.004 | 0.0152 | 0.00659 | 1.77 |
| 6A | 0.00259 | 0.426 | 0.411 | 0.302 | 2.37 |
| 6B | 0.000514 | 0.0362 | 0.0347 | 0.0265 | 2.79 |
| 7 | 0.00708 | 1.18 | 0.589 | 0.538 | 9.02 |
| 8 | 0.000397 | 0.00316 | 0.0193 | 0.00949 | 4.61 |
| 9 | 0.00522 | 0.958 | 0.364 | 0.351 | 4.01 |
| 10 | 0.00671 | 0.496 | 0.645 | 0.57 | 2.48 |
| 11 | 0.00177 | 0.303 | 0.139 | 0.177 | 2.89 |
| 12 | 0.00215 | 0.264 | 0.371 | 0.462 | 8.13 |
| 13A | 0.0007 | 0.0008 | 0.599 | 0.552 | 0.95 |
| 13B | 0.155 | 2.4 | | | |
| 14 | 0.00256 | 0.0366 | 0.365 | 0.0972 | 1.67 |
| 15 | 0.00112 | 0.0993 | 0.374 | 0.142 | 7.36 |
| 16A | 0.0982 | 8.41 | 1.08 | 1.53 | 1.23 |
| 16B | 0.00071 | 0.04 | 0.2 | 0.189 | 1.01 |
| 17 | 0.0005 | 0.00871 | 0.046 | 0.0315 | 1.83 |
| 18 | 0.0405 | 4.76 | 4.04 | 1.74 | 2.95 |
| 19 | 0.000384 | 0.0306 | 0.0688 | 0.0534 | 2.74 |
| 20A | 0.0252 | 0.896 | 2.7 | 1.95 | 2.51 |
| 20B | 0.00025 | 0.0139 | 0.0713 | 0.0982 | 1.07 |
| 21 | 0.00106 | 0.00996 | 0.0191 | 0.0105 | 6.28 |
| 22A | 0.356 | 39.7 | | | |
| 22B | 0.00317 | 0.88 | 0.508 | 0.369 | 3.05 |
| 23 | 0.0000679 | 0.00297 | 0.00508 | 0.00286 | 1.38 |
| 24 | 0.0581 | 1.43 | 2.01 | 1.01 | 1.19 |
| 25 | 0.0002 | 0.046 | 0.0205 | 0.011 | 1.04 |
| 26 | 0.00257 | 0.68 | 0.14 | 0.0929 | 7.55 |
| 27 | 0.00698 | 1.03 | 0.162 | 0.122 | 4.19 |
| 28 | 0.000118 | 0.0015 | 0.00671 | 0.00369 | 1.44 |
| 29 | 0.000117 | 0.00142 | 0.0101 | 0.00645 | 1.08 |
| 30A | 0.000228 | 0.0228 | 0.0154 | 0.00849 | 2.92 |
| 30B | 0.00056 | 0.0841 | 0.0517 | 0.0299 | 2.71 |
| 31 | 0.00772 | 1.24 | 0.274 | 0.191 | 7.46 |
| 32 | 0.0131 | 1.59 | 0.849 | 0.45 | 5.3 |
| 33 | 0.00706 | 0.626 | 0.215 | 0.173 | 8.07 |
| 34 | 0.00643 | 0.712 | 0.423 | 0.186 | 8.66 |
| 35 | 0.00015 | 0.0317 | 0.0386 | 0.0118 | 2.39 |
| 36 | 0.00013 | 0.00144 | 0.00289 | 0.00154 | 1.65 |
| 37 | 0.000134 | 0.00829 | 0.296 | 0.0273 | 1.17 |
| 38A | 0.00074 | 0.106 | 0.0559 | 0.0335 | 6.61 |
| 38B | 0.00559 | 0.417 | 0.127 | 0.0875 | 5.55 |
| 39 | 0.00019 | 0.0165 | 0.0206 | 0.00883 | 2.91 |
| 40 | 0.00014 | 0.00879 | 0.00643 | 0.00324 | 2.18 |
| 41A | 0.000115 | 0.00752 | 0.0071 | 0.00429 | 1.01 |
| 41B | 0.00868 | 0.506 | 0.113 | 0.065 | 1.03 |
| 42A | 0.0182 | 3.15 | 0.634 | 0.399 | 5.22 |
| 42B | 3.2 | 36 | 11.4 | 3.87 | 14.7 |
| 43A | 0.122 | 27.6 | 3.25 | 7.1 | 3.88 |
| 43B | 0.00033 | 0.0614 | 0.0735 | 0.0361 | 2.72 |
| 44 | 0.00181 | 0.179 | 0.645 | 0.516 | 2.73 |
| 45A | 0.309 | 8.42 | 1.36 | 1.1 | 1.09 |
| 45B | 0.000495 | 0.00741 | 0.0328 | 0.023 | 2.36 |
| 46 | 0.00037 | 0.0167 | 0.054 | 0.0174 | 3.12 |
| 47 | 0.0006 | 0.0186 | 0.0357 | 0.0177 | 2.87 |
| 48 | 0.00064 | 0.0149 | 0.0504 | 0.0254 | 3.05 |
| 49 | 0.000325 | 0.0115 | 0.568 | 0.258 | 7.96 |
| 50 | 0.00019 | 0.00484 | 0.0261 | 0.0112 | 2.11 |

All technical and scientific terms used herein have the same meaning. Efforts have been made to ensure accuracy with respect to numbers used (e.g. amounts, temperature, etc.) but some experimental errors and deviations should be accounted for.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

Where a range of values is provided, it is understood that each intervening value, to the tenth of the unit of the lower limit, unless the context clearly dictates otherwise, between the upper and lower limit of the range and any other stated or intervening value in that stated range, is encompassed herein. The upper and lower limits of these small ranges which can independently be included in the smaller rangers is also encompassed herein, subject to any specifically excluded limit in the stated range. Where the stated range includes one or both of the limits, ranges excluding either or both of those included limits are also included herein.

Many modifications and other embodiments of the compounds and methods set forth herein will come to mind to one skilled in the art having the benefit of the teachings presented in the foregoing descriptions and the associated drawings. Therefore, it is to be understood that the compounds and methods described herein are not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A compound having formula (I): 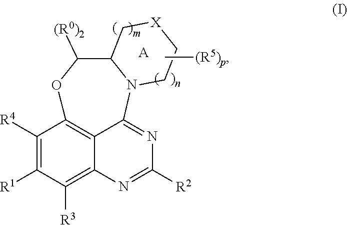
or a stereoisomer, atropisomer, tautomer, or pharmaceutically acceptable salt thereof, wherein;
X is O or NR⁶;
n is 1, 2, or 3;
m is 1,2, or 3;
p is 0, 1, or 2;
wherein n and m together make a 6-, 7-, or 8-membered ring A;
each R⁰ is independently hydrogen or methyl;
R¹ is R⁷-substituted or unsubstituted napthyl, R⁷-substituted or unsubstituted isoquinolinyl, R⁷-substituted or unsubstituted indazolyl, R⁷-substituted or unsubstituted benzothiazolyl, R^{7A}-substituted or unsubstituted phenyl, or R^{7A}-substituted or unsubstituted pyridinyl;
each R⁷ is independently hydrogen, halogen, —OH, NH₂, N(Me)₂, unsubstituted C₁₋₃ alkyl, unsubstituted C₁₋₃ haloalkyl, or unsubstituted cyclopropyl;
each R^{7A} is independently hydrogen, halogen, NH₂, N(Me)₂, unsubstituted C₁₋₃ alkyl, unsubstituted C₁₋₃ haloalkyl, or unsubstituted cyclopropyl;
R² is hydrogen, L¹-O-L²-R⁸, R^{8A}-substituted or unsubstituted C₁₋₃ alkyl, or R^{8B}-substituted or unsubstituted 4-10 membered heterocycle;
wherein when R² is hydrogen, R¹ is R⁷-substituted indazolyl, and n and m are 1, then p is not zero and R⁶ is not H;
L¹ is a bond or R^{L1}-substituted or unsubstituted C₁₋₃ alkylene;
R^{L1} is halogen or unsubstituted C₁₋₃ alkyl;
L² is a bond or unsubstituted C₁₋₃ alkylene;
R⁸ is R⁹-substituted or unsubstituted 4-10 membered heterocycle comprising N, S, or O;
each R⁹ is independently halogen, oxo, —OCF₃, —OCHF₂, —OCH₂F, unsubstituted C₁₋₃ alkyl, unsubstituted C₁₋₃ haloalkyl, unsubstituted C₁₋₃ alkoxy, R¹⁰-substituted or unsubstituted C₁₋₃ alkylidene, or R¹⁰-substituted or unsubstituted C₃₋₄ cycloalkyl, or R¹⁰-substituted or unsubstituted 3 or 4-membered heterocycle; or wherein
two R⁹ together form a R¹⁰-substituted or unsubstituted C₃₋₅ cycloalkyl or a R¹⁰-substituted or unsubstituted C₃₋₅ heterocycle comprising one or more oxygen atoms R¹⁰ is hydrogen, halogen, or C₁₋₃ unsubstituted alkyl;
each R^{8A} is independently R^{9A}-substituted or unsubstituted C₁₋₃ alkyl, R^{9A}-substituted or unsubstituted C₁₋₃ alkoxy, R^{9A}-substituted or unsubstituted C₃₋₄ cycloalkyl, or R^{9A}-substituted or unsubstituted 4-6 membered heterocycle;
each R^{9A} is independently halogen, oxo, unsubstituted C₁₋₃ alkyl, unsubstituted C₁₋₃ haloalkyl, unsubstituted C₁₋₃ alkoxy, unsubstituted C₁₋₃ alkylidene, R⁹-substituted or unsubstituted C₃₋₄ cycloalkyl, or R⁹-substituted or unsubstituted 4-10 membered heterocycle comprising N, S, or O;
R^{8B} is independently halogen, oxo, —NH₂, unsubstituted C₁₋₃ alkyl, unsubstituted C₁₋₃ haloalkyl, unsubstituted C₁₋₃ alkoxy, or unsubstituted C₁₋₃ alkylidene;
R³ and R⁴ are each independently hydrogen, —CN, halogen, unsubstituted C₁₋₃ alkyl, or unsubstituted cyclopropyl;
each R⁵ is independently halogen, oxo, unsubstituted C₁₋₃ alkyl, or unsubstituted C₁₋₃ haloalkyl; or wherein;
two R⁵ together form a bridge between two carbon atoms of ring A, wherein the bridge comprises 1-3 carbons and optionally one heteroatom selected from O and N; or
two R⁵ together form a bridge between two carbon atoms of ring A, wherein the bridge comprises one of O or NR¹¹;
R¹¹ is hydrogen, C(O)CH₃, or unsubstituted C₁₋₃ alkyl; and
R⁶ is hydrogen or R^{6A}-substituted or unsubstituted C₁₋₆ alkyl, R^{6A}-substituted or unsubstituted C₁₋₆ haloalkyl, R^{6A}-substituted or unsubstituted C₁₋₆ alkenyl; R^{6A}-substituted or unsubstituted C₁₋₆ alkynyl, or R^{6A}-substituted or unsubstituted 3-4 membered heterocycle;
R^{6A} is halogen, CN, OR^{6B}, SR^{6C}, S(O)₂R^{6C}, C(O)R^{6B}, unsubstituted C₁₋₃ alkyl; or, unsubstituted C₁₋₃ haloalkyl, R^{6B}-substituted or unsubstituted 3-4 membered heterocycle;
R^{6B} and R^{6C} are each independently C₁₋₃ alkyl or C₁₋₃ haloalkyl.

2. The compound of claim 1, wherein each R⁰ is hydrogen

3. The compound of claim 1, wherein one R⁰ is hydrogen and one R⁰ is methyl.

4. The compound of claim 3 having structure:

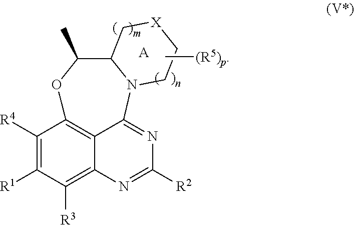

5. The compound of claim 1, wherein R¹ is R⁷-substituted or unsubstituted phenyl, R⁷-substituted or unsubstituted indazolyl, or R⁷-substituted or unsubstituted pyridinyl.

6. The compound of any one of claims 1-5, wherein R¹ is R⁷-substituted or unsubstituted phenyl.

7. The compound of any one of claims 1-5, wherein R¹ is R⁷-substituted or unsubstituted indazolyl.

8. The compound of any one of claims 1-5, wherein R¹ is R⁷-substituted or unsubstituted pyridinyl.

9. The compound of any one of claims 1-8, wherein each R⁷ is independently halogen, NH₂, unsubstituted C₁₋₃ alkyl, or unsubstituted C₁₋₃ haloalkyl.

10. The compound of any one of claims 1-5, wherein R¹ is 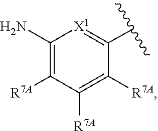
wherein,
X¹ is N or CF, and
R^{7A} is hydrogen, halogen, unsubstituted C₁₋₃ alkyl, or unsubstituted C₁₋₃ haloalkyl.

11. The compound of any one of claims 1, 5, 8, or 10, wherein R¹ is

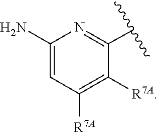

12. The compound of any one of claims 1, 5, 8, 10, or 11, wherein R¹ is

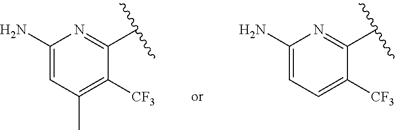 [SMILES: *c1nc(N)cc(C)c1C(F)(F)F ; *c1nc(N)ccc1C(F)(F)F]

13. The compound of any one of claims 1-6 or 10, wherein R¹ is 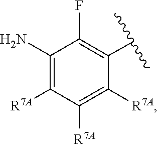
wherein R⁷ is hydrogen, halogen, unsubstituted C₁₋₃ alkyl or unsubstituted C₁₋₃ haloalkyl.

14. The compound of any one of claims 1-6, 10, or 13, wherein R¹ is

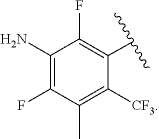 [SMILES: *c1c(F)c(N)c(F)c(C)c1C(F)(F)F]

15. The compound of any one of claims 1-5, wherein R¹ is 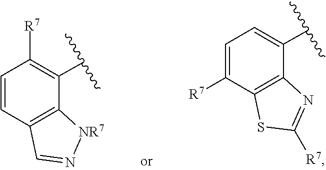
wherein each R⁷ is independently halogen, NH₂, N(Me)₂, or unsubstituted C₁₋₃ alkyl.

16. The compound of any one of claims 1-15, wherein R² is L¹-O-L²-R⁸, R^{8A}-substituted or unsubstituted C₁₋₃ alkyl, or R^{8B}-substituted or unsubstituted 4-6 membered heterocycle.

17. The compound of any one of claims 1-16, wherein R² is L¹-O-L²-R⁸.

18. The compound of any one of claims 1-17, wherein L¹ is a bond.

19. The compound of any one of claims 16-18, wherein L² is unsubstituted C₁₋₃ alkylene.

20. The compound of any one of claims 16-19, wherein R⁸ is 4-10 membered heterocycle comprising one N heteroatom.

21. The compound of any one of claims 16-20, wherein R⁸ is 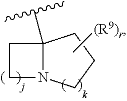
wherein,
R⁹ is halogen or R¹⁰-substituted or unsubstituted C₁₋₃ alkylidene
r is an integer of 0-12;
j is 1, 2, or 3; and
k is 1 or 2.

22. The compound of claim 21, wherein r is 0, 1, 2, or 3.

23. The compound of any one of claims 16-22, wherein R⁸ is 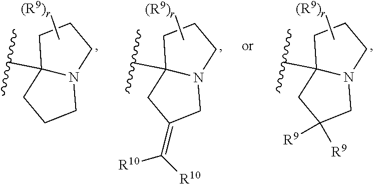
wherein,
R⁹ is independently halogen or R¹⁰-substituted or unsubstituted C₁₋₃ alkylidene;
each R¹⁰ is independently hydrogen or halogen; and
r is 1 or 2.

24. The compound of any one of claims 16-20, wherein R⁸ is 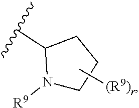
wherein,
R⁹ is independently halogen, oxo, or unsubstituted C₁₋₃ alkyl; and
r is 1 or 2.

25. The compound of any one of claims 16-20, wherein R⁸ is 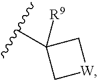
wherein
R⁹ is hydrogen or unsubstituted C₁₋₃ alkyl;
W is O, SO₂, or NR¹²; and
R¹² is hydrogen, unsubstituted C₁₋₃ alkyl, or unsubstituted C₁₋₃ haloalkyl.

26. The compound of any one of claims 16-20 or 25, wherein R⁸ is azetidinyl, oxetanyl, or thietanedioxide.

27. The compound of any one of claims 1-26, wherein R² is

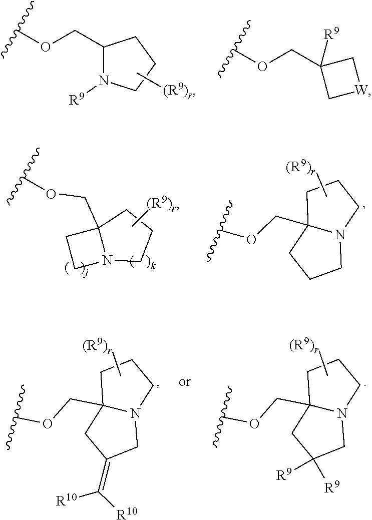

28. The compound of claim 27, wherein R⁹ is halogen or R¹⁰-substituted or unsubstituted C₁₋₃ alkylidene.

29. The compound of any one of claims 1-15, wherein R² is hydrogen.

30. The compound of any one of claims 1-16, wherein R² is R^{8A}-substituted or unsubstituted C₁₋₃ alkyl.

31. The compound of claim 29, wherein R^{8A} is independently R^{9A}-substituted or unsubstituted alkoxy or R^{9A}-substituted or unsubstituted 4-6 membered heterocycle.

32. The compound of claim 29 or claim 31, wherein R^{9A} is R⁹-substituted or unsubstituted 4-10 membered heterocycle comprising N.

33. The compound of claim 31 or 32, wherein R^{9A} is independently halogen, unsubstituted C₁₋₃ alkyl, or R¹⁰-substituted or unsubstituted C₁₋₃ alkylidene.

34. The compound of any one of claims 1-33, wherein R³ is halogen.

35. The compound of any one of claims 1-34, wherein R⁴ is hydrogen.

36. The compound of any one of claims 1-34, wherein R⁴ is halogen.

37. The compound of any one of claims 1-36, wherein R⁵ is independently oxo or unsubstituted C₁₋₃ alkyl, and p is 1.

38. The compound of any one of claims 1-36, wherein two R⁵ together form a bridge between two carbon atoms of ring A, wherein the bridge comprises 1-3 carbons.

39. The compound of claim 38, wherein the bridge comprises 1 carbon atom.

40. The compound of claim 38, wherein the bridge comprises 2 carbon atoms.

41. The compound of claim 1 having the formula: 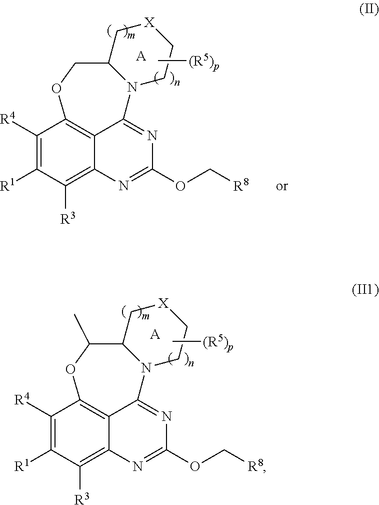
or a stereoisomer, atropisomer, tautomer, or pharmaceutically acceptable salt thereof.

42. The compound of claim 1 having the formula: 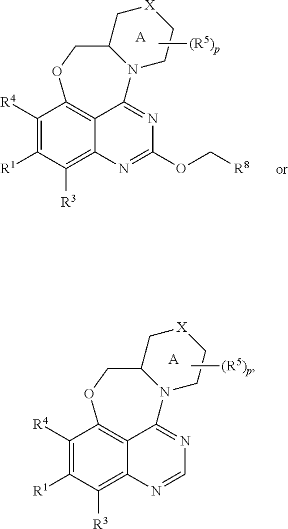
or a stereoisomer, atropisomer, tautomer, or pharmaceutically acceptable salt thereof.

43. The compound of claim 1 having the formula:

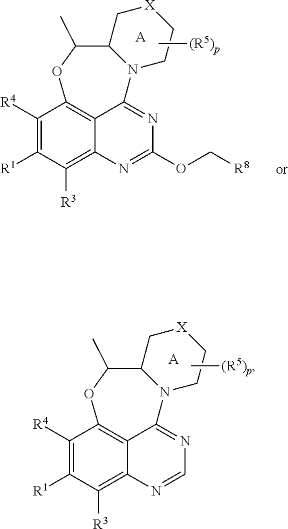

44. The compound of claim 1 having the formula: 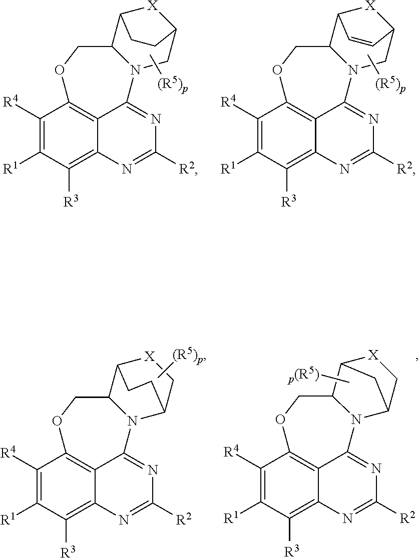 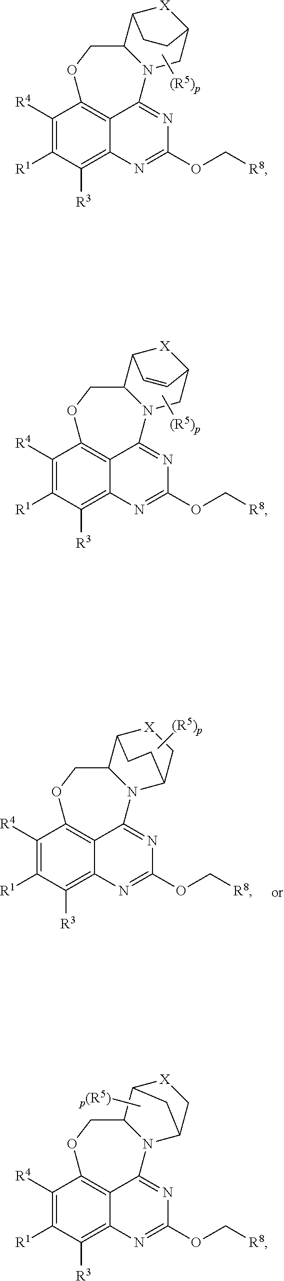
or a stereoisomer, atropisomer, tautomer, or pharmaceutically acceptable salt thereof.

45. The compound of claim 1 having the formula: 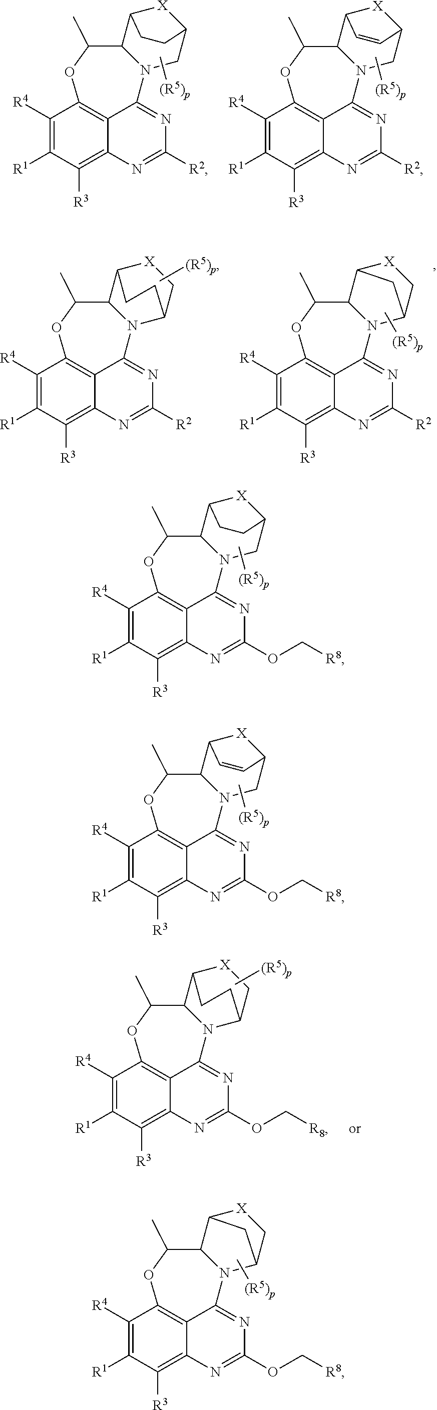
or a stereoisomer, atropisomer, tautomer, or pharmaceutically acceptable salt thereof.

46. The compound of claim 1 having the formula: 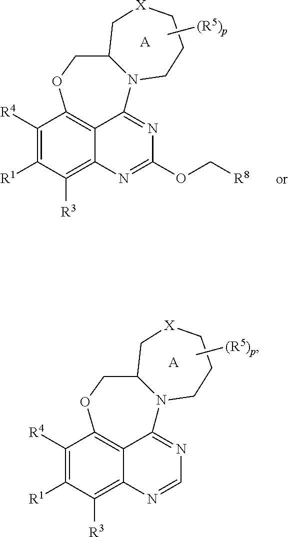
or a stereoisomer, atropisomer, tautomer, or pharmaceutically acceptable salt thereof.

47. The compound of claim 1 having the formula: 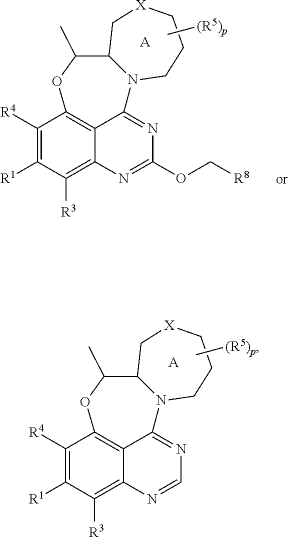
or a stereoisomer, atropisomer, tautomer, or pharmaceutically acceptable salt thereof.

48. The compound claim 1, having the formula: 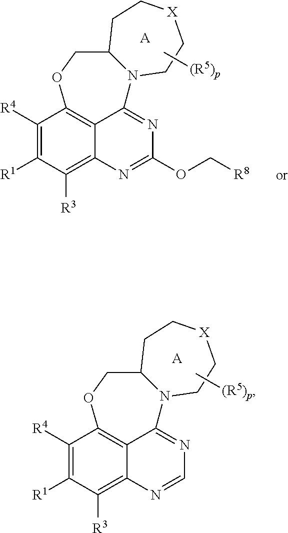
or a stereoisomer, atropisomer, tautomer, or pharmaceutically acceptable salt thereof.

49. The compound claim 1, having the formula: 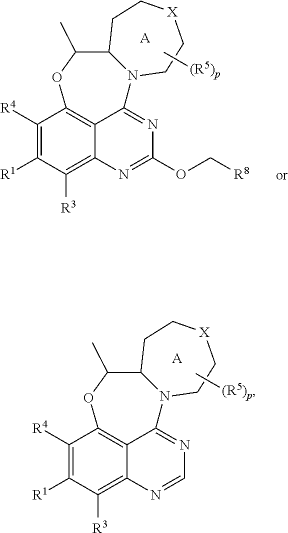
or a stereoisomer, atropisomer, tautomer, or pharmaceutically acceptable salt thereof.

50. The compound of any one of claims 1 or 41-49, wherein R⁸ is:

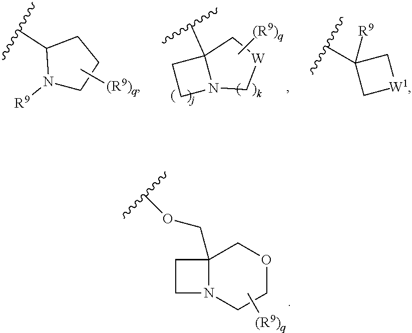

51. The compound of any one of claims 1 or 38-42, wherein R⁸ is:

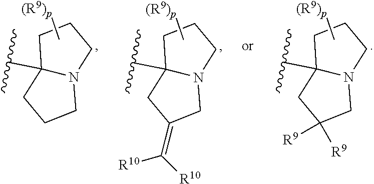

52. The compound of any one of claims 1 or 41-49, wherein R⁸ is:

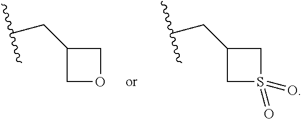 [SMILES: *CC1COC1 ; *CC1CS(=O)(=O)C1]

53. The compound of any one of claims 1-52, wherein X is NR⁶.

54. The compound claim 53, wherein R⁶ is R^{6A}-substituted or unsubstituted C₁₋₃ alkyl.

55. The compound claim 53, wherein R⁶ is R^{6A}-substituted C₁₋₃ alkyl.

56. The compound of claim 55, wherein R^{6A} is halogen, CN, OH, OMe, OEt, OCF₃, SO₂Me, unsubstituted C₁₋₃ alkyl, or 4-membered heterocycle.

57. The compound claim 53, wherein R⁶ is R^{6A}-substituted or unsubstituted C₁₋₃ haloalkyl.

58. The compound claim 53, wherein R⁶ is R^{6A}-substituted or unsubstituted C₁₋₃ alkenyl.

59. The compound claim 53, wherein R⁶ is R^{6A}-substituted or unsubstituted C₁₋₃ alkynyl.

60. The compound claim 53, wherein R⁶ is hydrogen.

61. The compound of claim 53, wherein R⁶ is methyl.

62. A compound of Table 1 or a stereoisomer, atropisomer, tautomer, or pharmaceutically acceptable salt thereof.

63. A compound of Table 2 or a stereoisomer, atropisomer, tautomer, or pharmaceutically acceptable salt thereof.

64. A compound of Table 3 or a stereoisomer, atropisomer, tautomer, or pharmaceutically acceptable salt thereof.

65. A pharmaceutical composition comprising a compound or a stereoisomer, atropisomer, tautomer, or pharmaceutically acceptable salt thereof of any one of claims 1-64 and one or more pharmaceutically acceptable excipients.

66. A method of treating cancer, the method comprising administering an effective amount of a compound or a stereoisomer, atropisomer, tautomer, or pharmaceutically acceptable salt thereof of any one of claims 1-64 or a pharmaceutical composition of claim 65.

67. The method of claim 66, wherein the cancer is characterized as comprising a KRas mutation.

68. The method of claim 67, wherein the KRas mutation corresponds to a KRas^{G12D} mutation.

69. The method of claim 68, further comprising testing a sample from the patient before administration for the absence or presence of a KRas^{G12D} mutation.

70. The method of claim 69, wherein the compound, stereoisomer, atropisomer, tautomer, or pharmaceutically acceptable salt thereof or pharmaceutical composition is administered to the patient after the patient sample shows the presence of a KRas^{G12D} mutation.

71. The method of any one of claims 66-70, wherein the cancer is tissue agnostic.

72. The method of any one of claims 66-70, wherein the cancer is pancreatic cancer, lung cancer, or colorectal cancer.

73. The method of claim 72, wherein the lung cancer is lung adenocarcinoma, NSCLC, or SCLC.

74. The method of claim 72, wherein the cancer is pancreatic cancer.

75. The method of claim 72, wherein the cancer is colorectal cancer.

76. The method of any one of claims 66-75, further comprising administering at least one additional therapeutic agent.

77. The method of claim 76, wherein the additional therapeutic agent comprises an epidermal growth factor receptor (EGFR) inhibitor, phosphatidylinositol kinase (PI3K) inhibitor, insulin-like growth factor receptor (IGF1R) inhibitor, a Janus kinase (JAK) inhibitor, a Met kinase inhibitor, a SRC family kinase inhibitor, a mitogen-activated protein kinase (MEK) inhibitor, an extracellular-signal-regulated kinase (ERK) inhibitor, a topoisomerase inhibitor, a taxane, an anti-metabolite agent, or an alkylating agent.

78. A compound according to any one of claims 1-64, or a stereoisomer, atropisomer, tautomer, or pharmaceutically acceptable salt thereof, for use as therapeutically active substance.

79. The use of a compound according to any one of claims 1-64, or stereoisomer, atropisomer, tautomer, or pharmaceutically acceptable salt thereof, for the therapeutic treatment of a cancer comprising a KRas^{G12D} mutation.

80. The use of a compound according to any one of claims 1-64, or stereoisomer, atropisomer, tautomer, or pharmaceutically acceptable salt thereof, for the preparation of a medicament for the therapeutic treatment of a cancer comprising a KRas^{G12D} mutation.

81. Use of a compound of any one of claims 1-64, or stereoisomer, atropisomer, tautomer, or pharmaceutically salt thereof, in the manufacture of a medicament for inhibiting tumor metastasis.

82. A compound according to any one of claims 1-64, or stereoisomer, atropisomer, tautomer, or pharmaceutically salt thereof, for the therapeutic and/or prophylactic treatment of a cancer comprising a KRas^{G12D} mutation.

83. A method for regulating activity of a KRas mutant protein, the method comprising reacting the mutant protein with a compound of any one of claims 1-64, or stereoisomer, atropisomer, tautomer, or pharmaceutically acceptable salt thereof.

84. A method for inhibiting proliferation of a cell population, the method comprising contacting the cell population with the compound of any one of claims 1-64, or stereoisomer, atropisomer, tautomer, or pharmaceutically acceptable salt thereof.

85. The method of claim 84, wherein the inhibition of proliferation is measured as a decrease in cell viability of the cell population.

86. A method for preparing a labeled KRas^{G12D} mutant protein, the method comprising reacting a KRas^{G12D} mutant protein with a labeled compound of any one of claims 1-64, or stereoisomer, atropisomer, tautomer, or pharmaceutically acceptable salt thereof, to result in the labeled KRas^{G12D} mutant protein.

87. A method for inhibiting tumor metastasis comprising administering to an individual in need thereof a therapeutically effective amount of the compound of any one of claims 1-64, or stereoisomer, atropisomer, tautomer, or pharmaceutically acceptable salt thereof or a pharmaceutical composition of claim 65 to a subject in need thereof.

88. A process for synthesizing a compound of formula (I) as set forth herein.
